(19) United States
(12) Patent Application Publication
(10) Pub. No.: US 2023/0320207 A1
(43) Pub. Date: Oct. 5, 2023

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Hyun KIM, Gyeonggi-do (KR); Hong-Se OH, Gyeonggi-do (KR); Mi-Ja LEE, Gyeonggi-do (KR); Ji-Won UM, Gyeonggi-do (KR); Kyoung-Jin PARK, Gyeonggi-do (KR); So-Mi PARK, Gyeonggi-do (KR); Hyun-Woo KANG, Gyeonggi-do (KR); Sang-Hee CHO, Gyeonggi-do (KR)

(21) Appl. No.: 18/075,353

(22) Filed: Dec. 5, 2022

(30) Foreign Application Priority Data
Jan. 5, 2022 (KR) 10-2022-0001358

(51) Int. Cl. H10K 85/60; H10K 85/40; C07D 405/14; C07D 405/04; C07D 405/10; C07D 407/10; C07D 409/10; C07D 311/82; C07D 409/14; C07D 311/88; H10K 50/11

(52) U.S. Cl. CPC H01L 51/0072; C07D 311/82; C07D 311/88; C07D 405/04; C07D 405/10; C07D 405/14; C07D 407/10; C07D 409/10; C07D 409/14; H01L 51/0054; H01L 51/0059; H01L 51/0061; H01L 51/0064; H01L 51/0067; H01L 51/0073; H01L 51/0074; H01L 51/0094; H01L 51/5012; C07B 2200/05

(57) ABSTRACT

The present disclosure relates to a plurality of host materials, an organic electroluminescent compound, and an organic electroluminescent device comprising the same. By comprising a specific combination of compounds according to the present disclosure as a plurality of host materials, or by comprising the compound according to the present disclosure, it is possible to produce an organic electroluminescent device having improved driving voltage, luminous efficiency and/or lifetime properties, compared to the conventional organic electroluminescent device.

### TECHNICAL FIELD

The present disclosure relates to a plurality of host materials, an organic electroluminescent compound, and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. In many applications such as TVs and lightings, the lifetime of OLEDs is insufficient and higher efficiency of OLEDs is still required. Typically, the higher the luminance of an OLED, the shorter the lifetime that the OLED has. Thus, an OLED which has high luminous efficiency and/or long lifetime is required for long time uses and high resolution of displays.

In order to enhance luminous efficiency, driving voltage and/or lifetime, various materials or concepts for an organic layer of an OLED have been proposed. However, they were not satisfactory in practical use. In addition, there has been a need to develop an organic electroluminescent material having more improved performances, for example, improved driving voltage, luminous efficiency, power efficiency, and/or lifetime properties compared to a combination of specific compounds previously disclosed.

Meanwhile, Korean Patent Application Laying-Open No. 2019-0066895 discloses a compound having a spiro[benzo[c]chromene-6,9′-fluorene] structure as a hole blocking material. However, the aforementioned reference does not specifically disclose a specific compound or a specific combination of host materials claimed in the present disclosure. In addition, there is still a need to develop a light-emitting material having more improved performances, for example, improved driving voltage, luminous efficiency, power efficiency, and/or lifetime properties compared to a combination of specific compounds previously disclosed.

### DISCLOSURE OF INVENTION

### Technical Problem

The objective of the present disclosure is to provide a plurality of host materials capable of providing an organic electroluminescent device having improved driving voltage, luminous efficiency and/or lifetime properties. Another objective of the present disclosure is to provide an organic electroluminescent compound having a new structure suitable for applying to an organic electroluminescent device. Still another objective of the present disclosure is to provide an organic electroluminescent device having improved driving voltage, luminous efficiency and/or lifetime properties by comprising a compound or a specific combination of compounds according to the present disclosure.

### Solution to Problem

As a result of intensive studies to solve the technical problems, the present inventors found that the above objective can be achieved by a plurality of host materials comprising a first host material comprising a compound represented by the following formula 1 and a second host material comprising a compound represented by the following formula 2, or by a compound represented by the following formula 5.

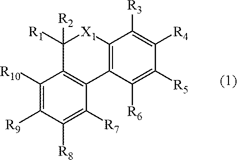

In formula 1,
X₁ represents -N=, —NR₀—, -O- or -S-;
R₀ represents a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di-(3- to 30-membered)heteroarylamino, or a substituted or unsubstituted (C6-C30)aryl(3- to 30-membered)heteroarylamino;
R₁ to R₁₀. each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di- (3- to 30-membered)heteroarylamino, or a substituted or unsubstituted (C6-C30)aryl(3- to 30-membered)heteroarylamino; or may be linked to an adjacent substituent(s) to form a ring(s);
with the proviso that at least one of R₃ to R₁₀ comprises —(L₁)ₐ—Ar₁;
Ar₁ represents a substituted or unsubstituted (C6-C30)aryl, a substituted or unsubstituted (3- to 30-membered)heteroaryl, or

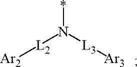

L₁ to L₃. each independently, represent a single bond, a substituted or unsubstituted (C6-C30)arylene, or a substituted or unsubstituted (3- to 30-membered)heteroarylene;
Ar₂ and Ar₃, each independently, represent a substituted or unsubstituted (C6-C30)aryl, a substituted or unsubstituted (3- to 30-membered)heteroaryl, or a substituted or unsubstituted (C3-C30)cycloalkyl; and
a represents an integer of 1 or 2, where if a is an integer of 2. each of L₁ may be the same or different.

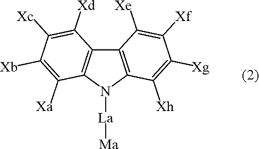

In formula 2,
Ma represents a substituted or unsubstituted (5- to 30-membered)heteroaryl containing a nitrogen(s);
La represents a single bond, a substituted or unsubstituted (C6-C30)arylene, or a substituted or unsubstituted (3- to 30-membered)heteroarylene;
Xa to Xh, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (3- to 30-membered)heteroarylamino, or a substituted or unsubstituted (C6-C30)aryl(3- to 30-membered)heteroarylamino; or may be linked to an adjacent substituent(s) to form any one ring selected from benzene, indole, indene, benzoindole, benzofuran, and benzothiophene, in which the ring may be further substituted with at least one of a (C1-C10)alkyl(s), and an (C6-C15)aryl(s) unsubstituted or substituted with a cyano(s) or a tri(C6-C18)arylsilyl(s); and
the heteroaryl comprises at least one heteroatom selected from B, N, O, S, Si and P.

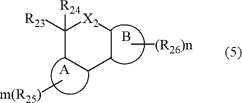

In formula 5,
X₂ represents -N=, —NR₀—, -O- or -S-;
R₀, R₂₃ and R₂₄, each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di- (3- to 30-membered)heteroarylamino, or a substituted or unsubstituted (C6-C30)aryl(3- to 30-membered)heteroarylamino;
A and B, each independently, represent a mono- or polycyclic, (C5-C20) alicyclic or aromatic ring, or the combination thereof, in which a carbon atom(s) of the ring may be replaced with at least one heteroatom selected from N, O, and S;
R₂₅ and R₂₈, each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di- (3- to 30-membered)heteroarylamino, a substituted or unsubstituted (C6-C30)aryl(3- to 30-membered)heteroarylamino, or —(L₁)ₐ—Ar₁;
with the proviso that at least one of R₂₅ and R₂₆ represents —(L₁)ₐ—Arₗ;
L₁, each independently, represents a single bond, a substituted or unsubstituted (C6-C30)arylene, or a substituted or unsubstituted (3- to 30-membered)heteroarylene;
Ar₁, represents a substituted or unsubstituted (C6-C30)aryl, a substituted or unsubstituted (3- to 30-membered)heteroaryl, or

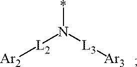

L₂ and L₃, each independently, represent a single bond, a substituted or unsubstituted (C6-C30)arylene, or a substituted or unsubstituted (3- to 30-membered)heteroarylene;
Ar₂ and Ar₃, each independently, represent a substituted or unsubstituted (C6-C30)aryl, a substituted or unsubstituted (3- to 30-membered)heteroaryl, or a substituted or unsubstituted (C3-C30)cycloalkyl;
m and n, each independently, represent an integer of 1 to 4, where if m and n are an integer of 2 or more, each of R₂₅ and each of R₂₆ may be the same or different; and
a represents an integer of 1 or 2, where if a is an integer of 2, each of L₁ may be the same or different.

### Advantageous Effects of Invention

An organic electroluminescent device having lower driving voltage, higher luminous efficiency, and/or improved lifetime properties compared to the conventional organic electroluminescent device is provided by comprising a specific combination of compounds according to the present disclosure as a plurality of host materials, or by comprising the compound according to the present disclosure, and it is possible to produce a display system or lighting system using the same.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure may be a combination of at least two host materials, which, optionally, may further comprise conventional materials included in the organic electroluminescent material. At least two compounds comprised in the plurality of host materials may be comprised together in one light-emitting layer, or each may be comprised in different light-emitting layers. For example, the at least two host materials may be mixture-evaporated or co-evaporated, or may be separately evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl” or “(C6-C30)arylene” is meant to be a monocyclic or fused ring radical derived from an aromatic hydrocarbon having 6 to 30 ring backbone carbon atoms, and may be partially saturated. The above aryl may comprise a spiro structure. The above aryl may include phenyl, biphenyl, terphenyl, quinquephenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, spiro[cyclopentene-fluorene]yl, spiro[dihydroindene-fluorene]yl, azulenyl, tetramethyl-dihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl. 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl. 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl. 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11 -diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11 -diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11.11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl. 9,9,10,10-tetramethyl-9.10-dihydro-1-phenanthrenyl, 9.9,10.10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl” or “(3- to 30-membered)heteroarylene” is meant to be an aryl or arylene having 3 to 30 ring backbone atoms, and including at least one heteroatom selected from the group consisting of B, N, O, S, Si, and P. The number of heteroatoms is preferably 1 to 4. The above heteroaryl or heteroarylene may be a monocyclic ring, or a fused ring condensed with at least one benzene ring; and may be partially saturated. In addition, the above heteroaryl or heteroarylene may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl. 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl. 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl. 2-methyl-3-indolyl, 4-methyl-3-indolyl, 2-tert-butyl-1-indolyl, 4-tertbutyl-1-indolyl. 2-fert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl. 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1.2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2.3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2.3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2.1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2.3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2.1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3.2-d]pyrimidinyl, 8-benzothio(3,2-d]pyrimidinyl. 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3.2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryl substituents are linked. Herein, the substituent(s) of the substituted alkyl, the substituted alkenyl, the substituted alkynyl, the substituted aryl, the substituted arylene, the substituted heteroaryl, the substituted heteroarylene, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di- alkylamino, the substituted mono- or di- alkenylamino, the substituted alkylalkenylamino, the substituted alkenylarylamino, the substituted alkenylheteroarylamino, the substituted alkylarylamino, the substituted mono- or di- arylamino, the substituted mono- or di- heteroarylamino, the substituted alkylheteroarylamino, the substituted arylheteroarylamino, and the substituted fused ring group of an aliphatic ring(s) and an aromatic ring(s), each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of deuterium, a halogen(s), a cyano(s), a (C1-C30)alkyl(s), a (C6-C30)aryl(s), a (3- to 30-membered)heteroaryl(s), and a tri(C6-C30)arylsilyl(s); a tri(C1-C30)alkylsilyl; a tri(C6-C30)arylsilyl; a di(C1-C30)alkyl(C6-C30)arylsityl; a (C1-C30)alkyldi(C6-C30)arylsilyl; an amino; a mono- or di- (C1-C30)alkylamino; a mono- or di- (C2-C30)alkenylamino; a mono- or di- (C6-C30)arylamino; a mono- or di- (3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3-to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a (C6-C30)arylphosphine; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a cyano; a (C1-C20)alkyl; a (5- to 25-membered)heteroaryl unsubstituted or substituted with a (C6-C25)aryl(s); a (C6-C25)aryl unsubstituted or substituted with at least one of deuterium, a cyano(s), a (C1-C20)alkyl(s), a (5- to 25-membered)heteroaryl(s), and a tri(C6-C25)arylsilyl(s); and a tri(C6-C25)arylsilyl. For example, the substituent(s), each independently, may be at least one selected from the group consisting of deuterium; a methyl; a substituted or unsubstituted phenyl; a naphthyl; a substituted or unsubstituted biphenyl; a terphenyl; a triphenylenyl; a phenylnaphthyl; a substituted or unsubstituted dibenzofuranyl; a dibenzothiophenyl; and a triphenylsilyl, in which the substituent(s) of the substituted phenyl, the substituted biphenyl, and the substituted dibenzofuranyl may be at least one of a phenyl, a cyano, a tert-butyl, a dibenzofuranyl, a dibenzothiophenyl, and a triphenylsilyl.

In the present disclosure, “a ring formed by a linkage of adjacent substituents” means that at least two adjacent substituents are linked or fused to each other to form a substituted or unsubstituted, mono- or polycyclic, (3- to 30-membered) alicyclic or aromatic ring, or the combination thereof. The ring may be preferably a substituted or unsubstituted, mono- or polycyclic, (3- to 26-membered) alicyclic or aromatic ring, or the combination thereof, and more preferably a mono- or polycyclic, (5- to 25-membered) aromatic ring unsubstituted or substituted with at least one of a (C1-C6)alkyl(s), a (C6-C18)aryl(s), and a (3- to 20-membered)heteroaryl(s). In addition, the formed ring may contain at least one heteroatom selected from B, N, O, S, Si, and P, preferably at least one heteroatom selected from N, O, and S. For example, the ring may be a benzene ring, a cyclopentane ring, an indene ring, an indane ring, a fluorene ring, a phenanthrene ring, an indole ring, a benzoindole ring, a benzofuran ring, a benzothiophene ring, a xanthene ring, etc., in which these rings may be substituted with at least one of a phenyl(s) unsubstituted or substituted with a cyano(s) or a triphenylsilyl(s), a biphenyl(s), and a methyl(s).

In the present disclosure, heteroaryl, heteroarylene, and heterocycloalkyl may, each independently, contain at least one heteroatom selected from the group consisting of B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted mono- or di- (C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

A plurality of host materials of the present disclosure comprise a first host material and a second host material, wherein the first host material comprises at least one compound represented by formula 1 and the second host material comprises at least one compound represented by formula 2. According to one embodiment of the present disclosure, the compound represented by formula 1 and the compound represented by formula 2 are different from each other.

According to one embodiment of the present disclosure, formula 1 is represented by any one of the following formulas 6 to 11.

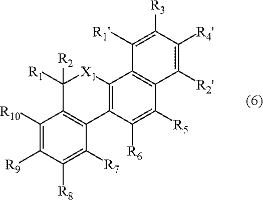

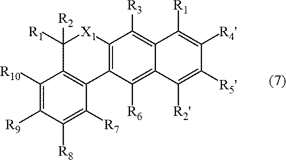

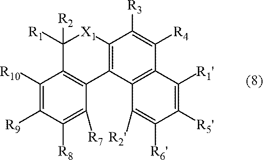

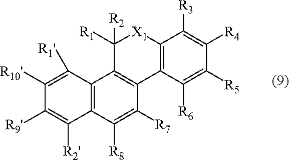

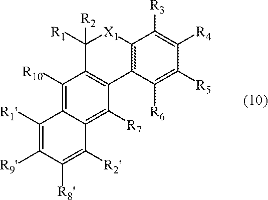

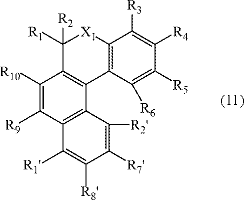

In formulas 6 to 11, X₁ represents -N=, —NR₀—, -O- or -S-. For example, X₁ may be -O-.

R₀ represents a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di-(3- to 30-membered)heteroarylamino, or a substituted or unsubstituted (C6-C30)aryl(3- to 30-membered)heteroarylamino.

In formulas 1 and 6 to 11, R₁ to R₁₀ and R_{1′} to R_{10′}, each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to an adjacent substituent(s) to form a ring(s). The ring may be a substituted or unsubstituted, mono- or polycyclic, (3- to 30-membered) alicyclic or aromatic ring, or the combination thereof, and may contain at least one heteroatom selected from B, N, O, S, Si, and P. According to one embodiment of the present disclosure, the ring may be a substituted or unsubstituted, mono- or polycyclic, (C3-C30) alicyclic or aromatic ring, and the carbon atom(s) of the formed alicyclic or aromatic ring may be replaced with at least one heteroatom selected from N, O, and S. The fused aromatic or heteroaromatic ring may be any one selected from benzene, indole, indene, benzofuran, and benzothiophene, in which these rings may be substituted with at least one of a (C1-C10)alkyl(s), and a (C6-C15)aryl(s) unsubstituted or substituted with a cyano(s) or a tri(C6-C18)arylsilyl(s). According to one embodiment of the present disclosure, R₁ and R₂, each independently, represent hydrogen, deuterium, a substituted or unsubstituted (C1-C20)alkyl, or a substituted or unsubstituted (C6-C25)aryl. According to another embodiment of the present disclosure, R₁ and R₂, each independently, represent hydrogen, deuterium, a (C1-C10)alkyl unsubstituted or substituted with deuterium, or an unsubstituted (C6-C18)aryl. For example, R₁ and R₂, each independently, may be hydrogen, deuterium, a methyl unsubstituted or substituted with deuterium, or a phenyl, etc. According to one embodiment of the present disclosure, R₃ to R₁₀, and R₁′ to R₁₀′, each independently, represent hydrogen, deuterium, a substituted or unsubstituted (C1-C20)alkyl, a substituted or unsubstituted (C6-C25)aryl, or —(L₁)ₐ—Ar_{1:} or may be linked to an adjacent substituent(s) to form a substituted or unsubstituted, mono- or polycyclic, (3- to 30-membered) alicyclic or aromatic ring, or the combination thereof. For example, in formula 1, R₃ to R₁₀, each independently, may be hydrogen or —(L₁)ₐ—Ar₁; or may be linked to an adjacent substituent(s) to form a benzene ring unsubstituted or substituted with —(L₁)ₐ—Ar₁. For example, in formulas 6 to 11, R₃ to R₁₀, and R₁′to R₁₀′, each independently, may be hydrogen or —(L₁)ₐ—Ar₁.

In formula 1, at least one, preferably any one of R₃ to R₁₀ comprises —(L₁)ₐ—Ar₁. According to one embodiment of the present disclosure, at least one, preferably any one of

R₃ to R₁₀ is —(L₁)ₐ—Ar₁; or R₃ to R₁₀ may be linked to an adjacent substituent(s) to form a ring substituted with —(L₁)ₐ—Ar₁. In formulas 6 to 11, at least one, preferably any one of R₃ to R₁₀, and R′ to R₁₀′ represents —(L₁)ₐ—Ar₁.

a represents an integer of 1 or 2, where if a is an integer of 2, each of L₁ may be the same or different.

L₁, each independently, represents a single bond, a substituted or unsubstituted (C6-C30)arylene, or a substituted or unsubstituted (3- to 30-membered)heteroarylene. According to one embodiment of the present disclosure, L₁, each independently, represents a single bond, a substituted or unsubstituted (C6-C18)arylene unsubstituted or substituted with a (C6-C18)aryl(s), or a (3- to 30-membered)heteroarylene unsubstituted or substituted with a (C6-C18)aryl(s). For example, L₁, each independently, represents a single bond; a phenylene unsubstituted or substituted with a phenyl(s); a naphthylene; a biphenylene; a pyridylene; or a carbazolylene unsubstituted or substituted with a phenyl(s), a biphenyl(s), or a naphthyl(s), etc.

Ar₁, represents a substituted or unsubstituted (C6-C30)aryl, a substituted or unsubstituted (3- to 30-membered)heteroaryl, or

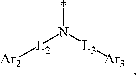

According to oneembodiment of the present disclosure, Ar₁, represents an unsubstituted (C6-C30)aryl, a (5- to 20-membered)heteroaryl unsubstituted or substituted with a (C6-C18)aryl(s) or a (5- to 20-membered)heteroaryl(s), or

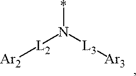

For example, Ar₁ may be a phenyl, a naphthyl, a biphenyl, a terphenyl, a triphenylenyl, a naphthylphenyl, a phenylnaphthyl, a quinquephenyl, a substituted or unsubstituted carbazolyl, a substituted or unsubstituted triazinyl, a substituted pyrimidinyl, a substituted pyridyl, a dibenzofuranyl, a dibenzothiophenyl, or

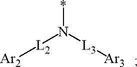

the substituent(s) of the substituted carbazolyl, the substituted triazinyl, the substituted pyrimidinyl, and the substituted pyridyl, each independently, may be at least one selected from the group consisting of a phenyl unsubstituted or substituted with a dibenzofuranyl(s) or dibenzothiophenyl(s), a biphenyl, a naphthyl, a terphenyl, a triphenylenyl, a phenylnaphthyl, a dibenzofuranyl unsubstituted or substituted with a phenyl(s), and a dibenzothiophenyl.

L₂ and L₃, each independently, represent a single bond, a substituted or unsubstituted (C6-C30)arylene,or a substituted or unsubstituted (3- to 30-membered)heteroarylene. According to one embodiment of the present disclosure, L₂ and L₃, each independently, represent a single bond, or an unsubstituted (C6-C18)arylene. For example, L₂ and L₃ may be a single bond.

Ar₂ and Ar₃, each independently, represent a substituted or unsubstituted (C6-C30)aryl, a substituted or unsubstituted (3- to 30-membered)heteroaryl, or a substituted or unsubstituted (C3-C30)cycloalkyl. According to one embodiment of the present disclosure, Ar₂ and Ar₃, each independently, represent a (C6-C25)aryl unsubstituted or substituted with a (C1-C30)alkyl(s). For example, Ar₂ and Ar₃, each independently, may be a phenyl, a biphenyl, a terphenyl, or a dimethylfluorenyl, etc.

According to one embodiment of the present disclosure, formula 2 may be represented by the following formula 3 or 4:

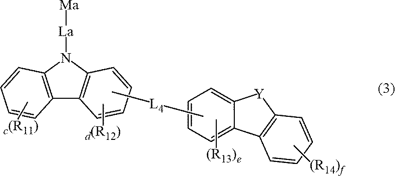

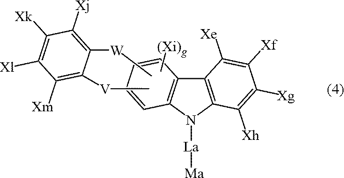

In formulas 2 to 4, Ma represents a substituted or unsubstituted (5- to 30-membered) heteroaryl containing a nitrogen(s). According to one embodiment of the present disclosure, Ma represents a substituted or unsubstituted (5- to 25-membered)heteroaryl containing a nitrogen(s), in which the substituent(s) of the heteroaryl may be at least one of a cyano, a (C6-C30)aryl, a (3- to 30-membered)heteroaryl, and a tri(C6-C30)arylsilyl. According to another embodiment of the present disclosure, Ma represents a substituted or unsubstituted (6- to 10-membered)heteroaryl containing a nitrogen(s), which may contain 1 to 3 nitrogens. Specifically, Ma represents a substituted or unsubstituted pyridyl, a substituted or unsubstituted pyrimidinyl, a substituted or unsubstituted triazinyl, a substituted or unsubstituted pyrazinyl, a substituted or unsubstituted pyridazinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, or a substituted or unsubstituted benzothienopyrimidinyl. For example, Ma may be a substituted or unsubstituted, quinazolinyl, pyrimidinyl, triazinyl, quinoxalinyl, naphthyridinyl, or quinolyl, etc., in which substituent(s) thereof may be at least one of a phenyl unsubstituted or substituted with a tert-butyl(s), a cyano(s), or a triphenylsilyl(s); a biphenyl unsubstituted or substituted with a cyano(s) or a triphenylsilyl(s); a naphthyl; a terphenyl; a dibenzothiophenyl; and a dibenzofuranyl.

In formulas 2 to 4. La represents a single bond, a substituted or unsubstituted (C6-C30)arylene, or a substituted or unsubstituted (3- to 30-membered)heteroarylene. According to one embodiment of the present disclosure. La represents a single bond; a (C6-C25)arylene unsubstituted or substituted with a (C6-C25)aryl(s) or a tri(C6-C25)arylsilyl(s); or an unsubstituted (5- to 20-membered)heteroarylene. For example, La may be a single bond; a phenylene unsubstituted or substituted with a phenyl(s), a biphenyl(s), or a triphenylsilyl(s); a biphenylene; a naphthylene; or a pyridylene, etc.

In formulas 2 and 4, Xa to Xi, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered) heteroarylamino, a substituted or unsubstituted mono- or di- (3- to 30-membered)heteroarylamino, or a substituted or unsubstituted (C6-C30)aryl(3- to 30-membered)heteroarylamino; or may be linked to an adjacent substituent(s) to form a ring(s). According to one embodiment of the present disclosure, Xa to Xi, each independently, represent hydrogen; deuterium; a cyano; a (C6-C30)aryl unsubstituted or substituted with a (5- to 20-membered)heteroaryl(s) or a tri(C6-C18)arylsilyl(s); a (5- to 25-membered)heteroaryl unsubstituted or substituted with a (C6-C25)aryl(s); or an unsubstituted tri(C6-C25)arylsilyl; or may be linked to an adjacent substituent(s) to form a ring(s). The ring(s) may be selected from benzene, indole, indene, benzofuran, benzothiophene, and benzoindole, in which the ring may be further substituted with at least one of a (C1-C10)alkyl(s), and a (C6-C15)aryl(s) unsubstituted or substituted with a cyano(s) or a tri(C6-C18)arylsilyl(s). In formula 2 or 4, any two adjacent ones of Xa to Xi may be linked to each other to form one or two rings. For example, Xa to Xi, each independently, may be hydrogen; a cyano; a phenyl unsubstituted or substituted with a dibenzofuranyl(s) or a triphenylsilyl(s); a biphenyl; a dibenzofuranyl unsubstituted or substituted with a phenyl(s); a dibenzothiophenyl unsubstituted or substituted with a phenyl(s); a phenylcarbazolyl unsubstituted or substituted with a cyano(s); a benzocarbazolyl substituted with a phenyl(s); or a triphenylsilyl, etc.; or any two adjacent ones of Xa to Xi may be linked to each other to form a benzene ring; a substituted indole ring; a substituted indene ring; a benzothiophene ring; a benzofuran ring; a benzoindole ring substituted with a phenyl(s), etc., in which the substituent(s) of the rings may be at least one of a phenyl unsubstituted or substituted with a cyano(s) or a triphenylsilyl(s), a biphenyl, and a methyl.

In formula 3, Y represents -S-, -O-, or —N(L₅—A₁)—.

In formula 3, L₄ and L₅, each independently, represent a single bond, or a substituted or unsubstituted (C6-C30)arylene. According to one embodiment of the present disclosure, L₄ and Ls, each independently, represent a single bond, or an unsubstituted (C6-C18)arylene. For example, L₄ and L₅. each independently, may be a single bond, or a phenylene.

A₁ represents a substituted or unsubstituted (C6-C30)aryl, or a substituted or unsubstituted (3- to 30-membered)heteroaryl. According to one embodiment of the present disclosure, A₁ represents a substituted or unsubstituted (C6-C25)aryl. For example, A₁ may be a phenyl unsubstituted or substituted with a cyano(s), etc.

In formula 3, R₁₁ and R₁₂, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (3- to 30-membered)heteroarylamino, or a substituted or unsubstituted (C6-C30)aryl(3- to 30-membered)heteroarylamino.

In formulas 3 and 4, R₁₃, R₁₄, and Xj to Xm, each independently, represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted fused ring group of a (C3-C30) aliphatic ring(s) and a (C6-C30) aromatic ring(s), -NR₁₆R₁₉, or -SiR₂₀R₂₁R₂₂; or may be linked to an adjacent substituent(s) to form a ring(s). For example, R₁₃ may be hydrogen or a phenyl, etc.; R₁₄, each independently, may be hydrogen or a phenyl, etc., or adjacent R₁₄′ₛ may be linked to each other to form a benzene ring(s); Xj to Xm, each independently, may be hydrogen or a phenyl, etc., or any two adjacent ones of Xj to Xm may be linked to each other to form a benzene ring(s).

R₁₈ to R₂₂, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s); or may be linked to an adjacent substituent(s) to form a ring(s).

In formula 3, c and f, each independently, represent an integer of 1 to 4, and d and e, each independently, represent an integer of 1 to 3, where if c to f are an integer of 2 or more, each of R₁₁ to each of R₁₄ may be the same or different.

In formula 4, g represents an integer of 1 or 2, where if g is 2, each of Xi may be the same or different.

In formula 4, V and W, each independently, represent a single bond, NR₁₅, CR₁₆R_{I7}, S, or O, with the proviso that both of V and W are not a single bond, or NR₁₅. According to one embodiment of the present disclosure, any one of V and W represents a single bond, and the other represents NR₁₅, CR₁₆R₁₇, S, or O.

R₁₅ to R₁₇, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30)aromatic ring(s). According to one embodiment of the present disclosure, R₁₅ to R₁₇, each independently, represent an unsubstituted (C1-C10)alkyl; or a (C6-C25)aryl unsubstituted or substituted with a cyano(s) or a tri(C6-C25)arylsityl(s). For example, R₁₅ may be a phenyl unsubstituted or substituted with a cyano(s) or a triphenylsilyl(s), or a biphenyl, etc., and R₁₈ and R₁₇, each independently, may be a methyl or a phenyl, etc.

The compound represented by formula 1 may be at least one selected from the group consisting of the following compounds, but is not limited thereto.

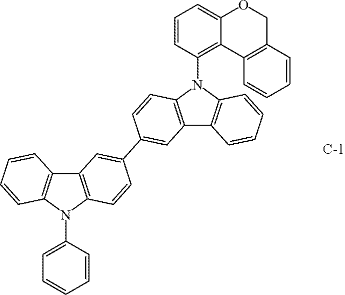 [SMILES: c1ccc(-n2c3ccccc3c3cc(-c4ccc5c(c4)c4ccccc4n5-c4cccc5c4-c4ccccc4CO5)ccc32)cc1]

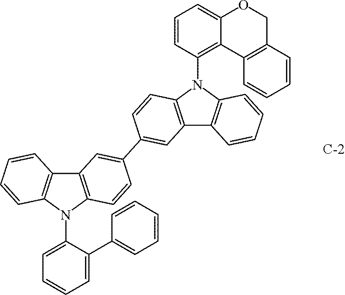 [SMILES: c1ccc(-c2ccccc2-n2c3ccccc3c3cc(-c4ccc5c(c4)c4ccccc4n5-c4cccc5c4-c4ccccc4CO5)ccc32)cc1]

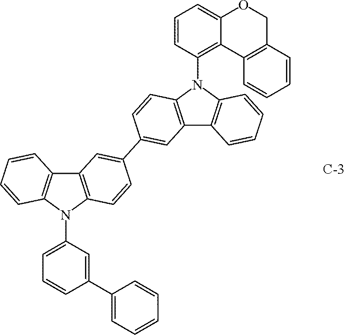 [SMILES: c1ccc(-c2cccc(-n3c4ccccc4c4cc(-c5ccc6c(c5)c5ccccc5n6-c5cccc6c5-c5ccccc5CO6)ccc43)c2)cc1]

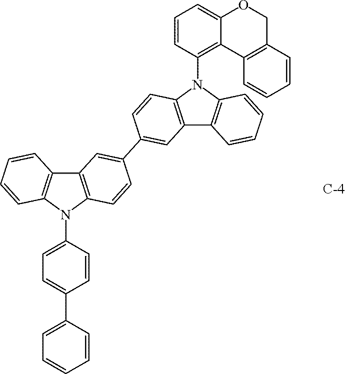 [SMILES: c1ccc(-c2ccc(-n3c4ccccc4c4cc(-c5ccc6c(c5)c5ccccc5n6-c5cccc6c5-c5ccccc5CO6)ccc43)cc2)cc1]

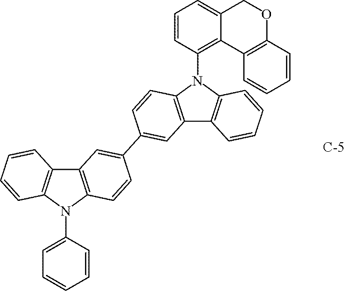 [SMILES: c1ccc(-n2c3ccccc3c3cc(-c4ccc5c(c4)c4ccccc4n5-c4cccc5c4-c4ccccc4OC5)ccc32)cc1]

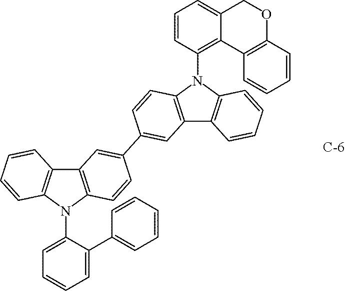 [SMILES: c1ccc(-c2ccccc2-n2c3ccccc3c3cc(-c4ccc5c(c4)c4ccccc4n5-c4cccc5c4-c4ccccc4OC5)ccc32)cc1]

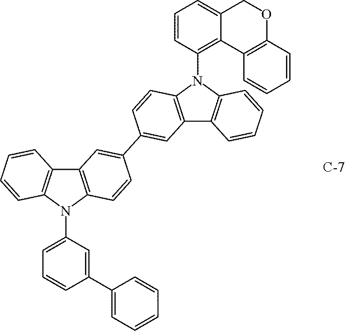 [SMILES: c1ccc(-c2cccc(-n3c4ccccc4c4cc(-c5ccc6c(c5)c5ccccc5n6-c5cccc6c5-c5ccccc5OC6)ccc43)c2)cc1]

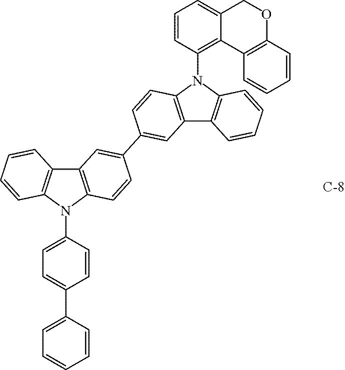 [SMILES: c1ccc(-c2ccc(-n3c4ccccc4c4cc(-c5ccc6c(c5)c5ccccc5n6-c5cccc6c5-c5ccccc5OC6)ccc43)cc2)cc1]

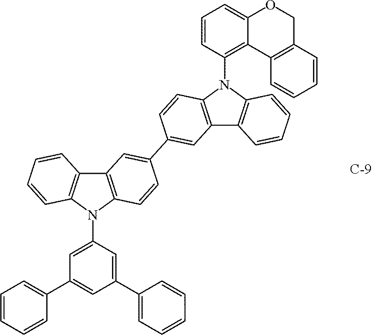 [SMILES: c1ccc(-c2cc(-c3ccccc3)cc(-n3c4ccccc4c4cc(-c5ccc6c(c5)c5ccccc5n6-c5cccc6c5-c5ccccc5CO6)ccc43)c2)cc1]

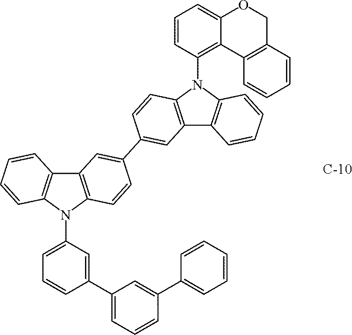 [SMILES: c1ccc(-c2cccc(-c3cccc(-n4c5ccccc5c5cc(-c6ccc7c(c6)c6ccccc6n7-c6cccc7c6-c6ccccc6CO7)ccc54)c3)c2)cc1]

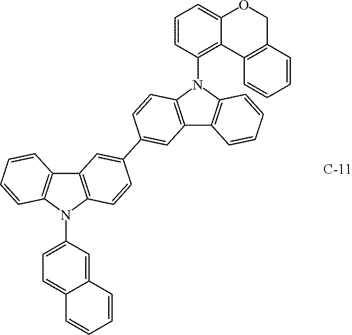 [SMILES: c1ccc2c(c1)COc1cccc(-n3c4ccccc4c4cc(-c5ccc6c(c5)c5ccccc5n6-c5ccc6ccccc6c5)ccc43)c1-2]

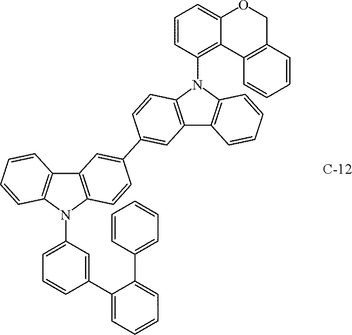 [SMILES: c1ccc(-c2ccccc2-c2cccc(-n3c4ccccc4c4cc(-c5ccc6c(c5)c5ccccc5n6-c5cccc6c5-c5ccccc5CO6)ccc43)c2)cc1]

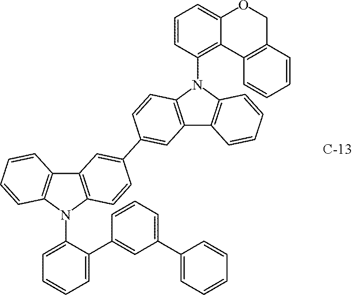 [SMILES: c1ccc(-c2cccc(-c3ccccc3-n3c4ccccc4c4cc(-c5ccc6c(c5)c5ccccc5n6-c5cccc6c5-c5ccccc5CO6)ccc43)c2)cc1]

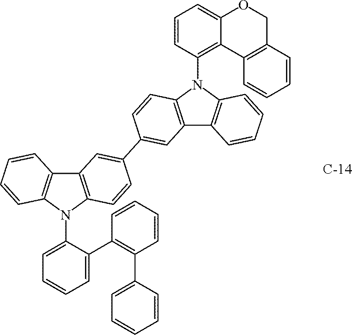 [SMILES: c1ccc(-c2ccccc2-c2ccccc2-n2c3ccccc3c3cc(-c4ccc5c(c4)c4ccccc4n5-c4cccc5c4-c4ccccc4CO5)ccc32)cc1]

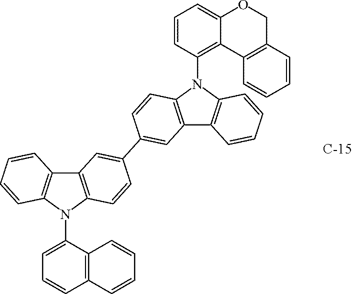 [SMILES: c1ccc2c(c1)COc1cccc(-n3c4ccccc4c4cc(-c5ccc6c(c5)c5ccccc5n6-c5cccc6ccccc56)ccc43)c1-2]

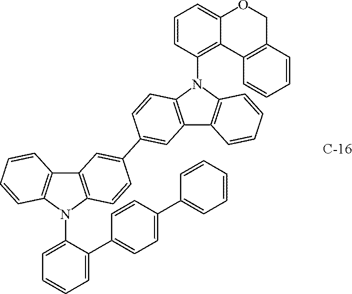 [SMILES: c1ccc(-c2ccc(-c3ccccc3-n3c4ccccc4c4cc(-c5ccc6c(c5)c5ccccc5n6-c5cccc6c5-c5ccccc5CO6)ccc43)cc2)cc1]

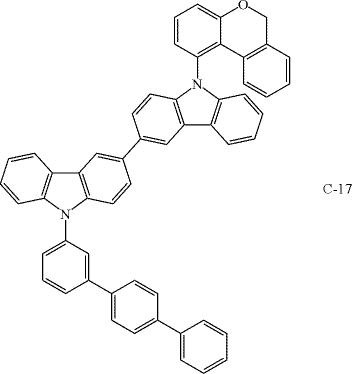 [SMILES: c1ccc(-c2ccc(-c3cccc(-n4c5ccccc5c5cc(-c6ccc7c(c6)c6ccccc6n7-c6cccc7c6-c6ccccc6CO7)ccc54)c3)cc2)cc1]

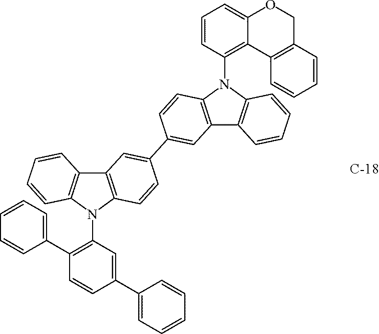 [SMILES: c1ccc(-c2ccc(-c3ccccc3)c(-n3c4ccccc4c4cc(-c5ccc6c(c5)c5ccccc5n6-c5cccc6c5-c5ccccc5CO6)ccc43)c2)cc1]

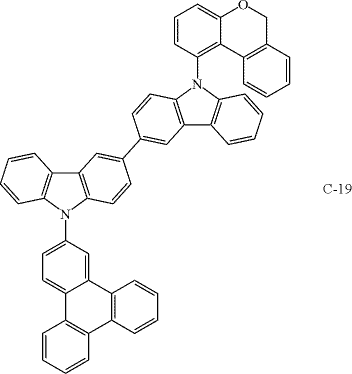 [SMILES: c1ccc2c(c1)COc1cccc(-n3c4ccccc4c4cc(-c5ccc6c(c5)c5ccccc5n6-c5ccc6c7ccccc7c7ccccc7c6c5)ccc43)c1-2]

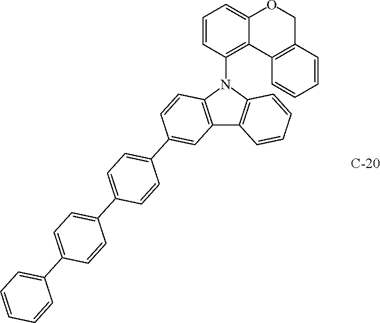 [SMILES: c1ccc(-c2ccc(-c3ccc(-c4ccc5c(c4)c4ccccc4n5-c4cccc5c4-c4ccccc4CO5)cc3)cc2)cc1]

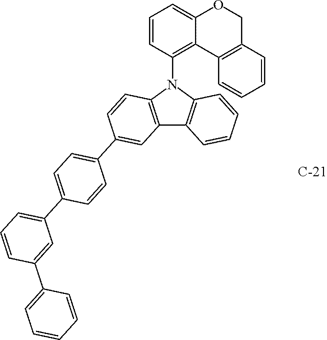 [SMILES: c1ccc(-c2cccc(-c3ccc(-c4ccc5c(c4)c4ccccc4n5-c4cccc5c4-c4ccccc4CO5)cc3)c2)cc1]

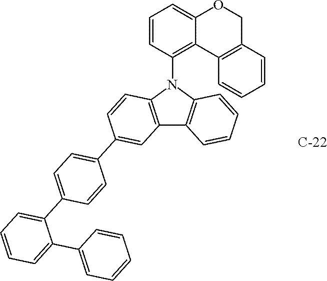 [SMILES: c1ccc(-c2ccccc2-c2ccc(-c3ccc4c(c3)c3ccccc3n4-c3cccc4c3-c3ccccc3CO4)cc2)cc1]

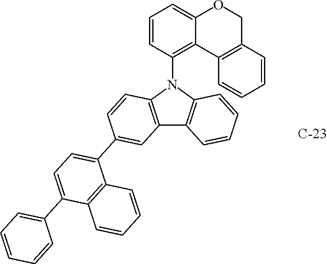 [SMILES: c1ccc(-c2ccc(-c3ccc4c(c3)c3ccccc3n4-c3cccc4c3-c3ccccc3CO4)c3ccccc23)cc1]

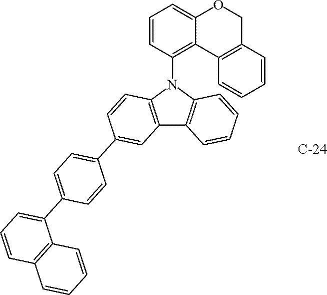 [SMILES: c1ccc2c(c1)COc1cccc(-n3c4ccccc4c4cc(-c5ccc(-c6cccc7ccccc67)cc5)ccc43)c1-2]

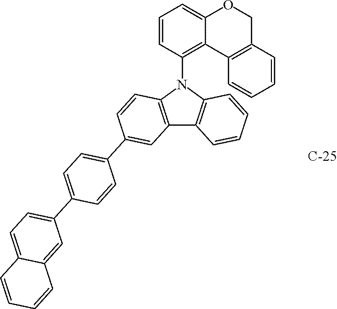 [SMILES: c1ccc2c(c1)COc1cccc(-n3c4ccccc4c4cc(-c5ccc(-c6ccc7ccccc7c6)cc5)ccc43)c1-2]

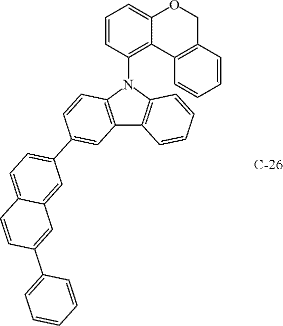 [SMILES: c1ccc(-c2ccc3ccc(-c4ccc5c(c4)c4ccccc4n5-c4cccc5c4-c4ccccc4CO5)cc3c2)cc1]

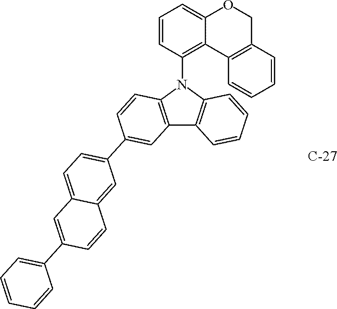 [SMILES: c1ccc(-c2ccc3cc(-c4ccc5c(c4)c4ccccc4n5-c4cccc5c4-c4ccccc4CO5)ccc3c2)cc1]

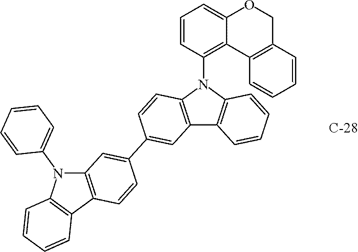 [SMILES: c1ccc(-n2c3ccccc3c3ccc(-c4ccc5c(c4)c4ccccc4n5-c4cccc5c4-c4ccccc4CO5)cc32)cc1]

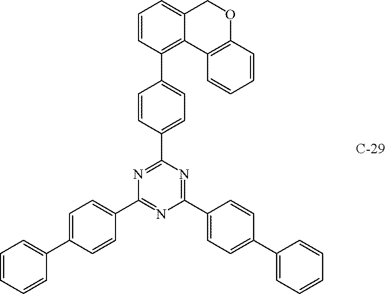 [SMILES: c1ccc(-c2ccc(-c3nc(-c4ccc(-c5ccccc5)cc4)nc(-c4ccc(-c5cccc6c5-c5ccccc5OC6)cc4)n3)cc2)cc1]

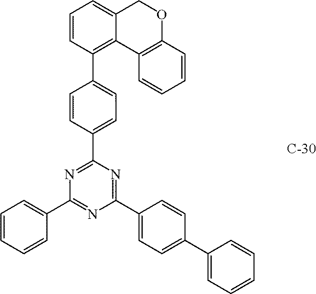 [SMILES: c1ccc(-c2ccc(-c3nc(-c4ccccc4)nc(-c4ccc(-c5cccc6c5-c5ccccc5OC6)cc4)n3)cc2)cc1]

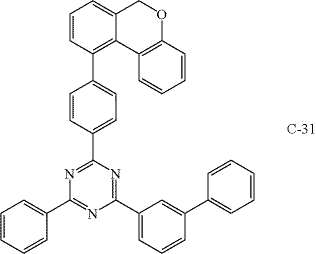 [SMILES: c1ccc(-c2cccc(-c3nc(-c4ccccc4)nc(-c4ccc(-c5cccc6c5-c5ccccc5OC6)cc4)n3)c2)cc1]

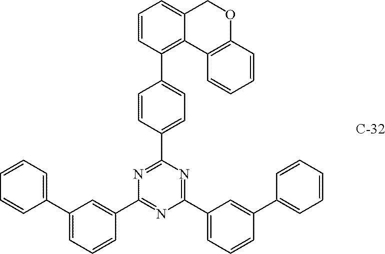 [SMILES: c1ccc(-c2cccc(-c3nc(-c4ccc(-c5cccc6c5-c5ccccc5OC6)cc4)nc(-c4cccc(-c5ccccc5)c4)n3)c2)cc1]

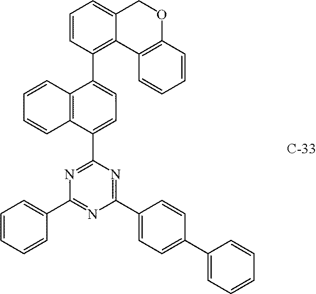 [SMILES: c1ccc(-c2ccc(-c3nc(-c4ccccc4)nc(-c4ccc(-c5cccc6c5-c5ccccc5OC6)c5ccccc45)n3)cc2)cc1]

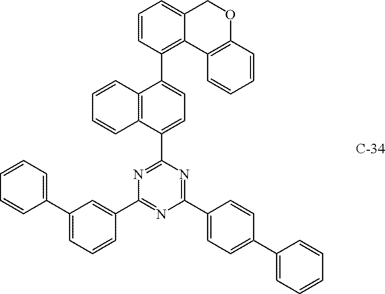 [SMILES: c1ccc(-c2ccc(-c3nc(-c4cccc(-c5ccccc5)c4)nc(-c4ccc(-c5cccc6c5-c5ccccc5OC6)c5ccccc45)n3)cc2)cc1]

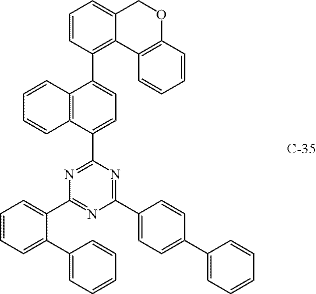 [SMILES: c1ccc(-c2ccc(-c3nc(-c4ccccc4-c4ccccc4)nc(-c4ccc(-c5cccc6c5-c5ccccc5OC6)c5ccccc45)n3)cc2)cc1]

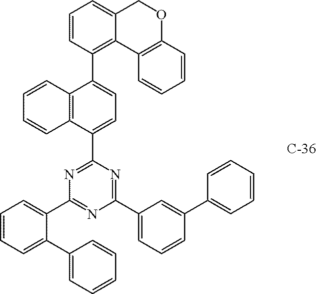 [SMILES: c1ccc(-c2cccc(-c3nc(-c4ccccc4-c4ccccc4)nc(-c4ccc(-c5cccc6c5-c5ccccc5OC6)c5ccccc45)n3)c2)cc1]

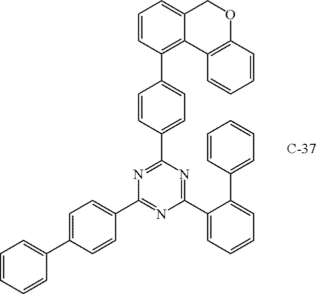 [SMILES: c1ccc(-c2ccc(-c3nc(-c4ccc(-c5cccc6c5-c5ccccc5OC6)cc4)nc(-c4ccccc4-c4ccccc4)n3)cc2)cc1]

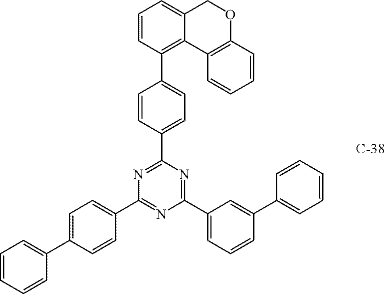 [SMILES: c1ccc(-c2ccc(-c3nc(-c4ccc(-c5cccc6c5-c5ccccc5OC6)cc4)nc(-c4cccc(-c5ccccc5)c4)n3)cc2)cc1]

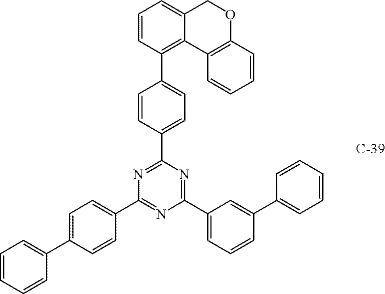 [SMILES: c1ccc(-c2ccc(-c3nc(-c4ccc(-c5cccc6c5-c5ccccc5OC6)cc4)nc(-c4cccc(-c5ccccc5)c4)n3)cc2)cc1]

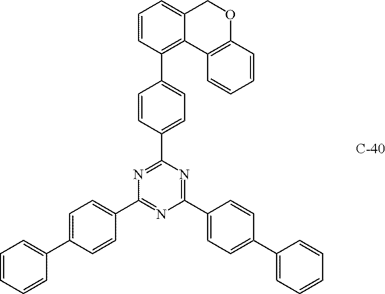 [SMILES: c1ccc(-c2ccc(-c3nc(-c4ccc(-c5ccccc5)cc4)nc(-c4ccc(-c5cccc6c5-c5ccccc5OC6)cc4)n3)cc2)cc1]

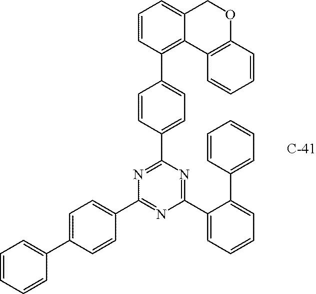 [SMILES: c1ccc(-c2ccc(-c3nc(-c4ccc(-c5cccc6c5-c5ccccc5OC6)cc4)nc(-c4ccccc4-c4ccccc4)n3)cc2)cc1]

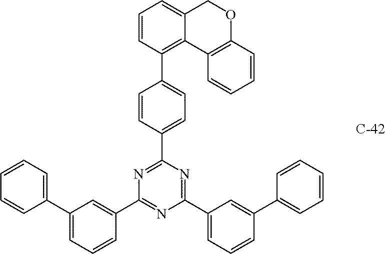 [SMILES: c1ccc(-c2cccc(-c3nc(-c4ccc(-c5cccc6c5-c5ccccc5OC6)cc4)nc(-c4cccc(-c5ccccc5)c4)n3)c2)cc1]

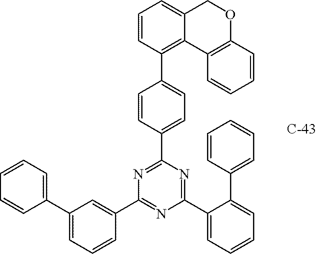 [SMILES: c1ccc(-c2cccc(-c3nc(-c4ccc(-c5cccc6c5-c5ccccc5OC6)cc4)nc(-c4ccccc4-c4ccccc4)n3)c2)cc1]

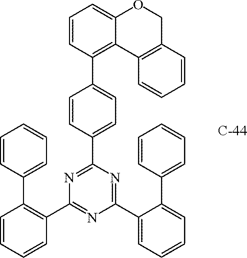 [SMILES: c1ccc(-c2ccccc2-c2nc(-c3ccc(-c4cccc5c4-c4ccccc4CO5)cc3)nc(-c3ccccc3-c3ccccc3)n2)cc1]

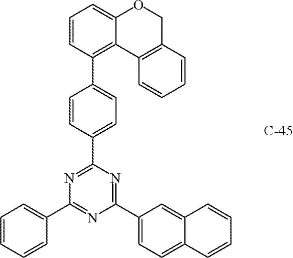 [SMILES: c1ccc(-c2nc(-c3ccc(-c4cccc5c4-c4ccccc4CO5)cc3)nc(-c3ccc4ccccc4c3)n2)cc1]

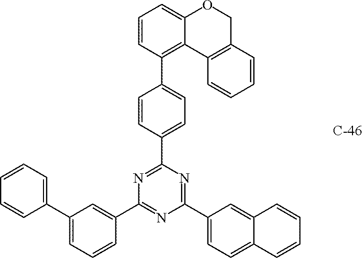 [SMILES: c1ccc(-c2cccc(-c3nc(-c4ccc(-c5cccc6c5-c5ccccc5CO6)cc4)nc(-c4ccc5ccccc5c4)n3)c2)cc1]

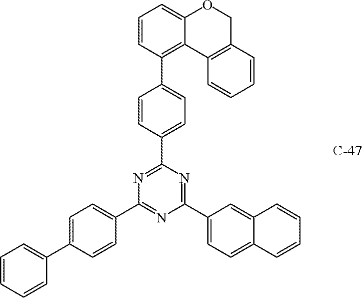 [SMILES: c1ccc(-c2ccc(-c3nc(-c4ccc(-c5cccc6c5-c5ccccc5CO6)cc4)nc(-c4ccc5ccccc5c4)n3)cc2)cc1]

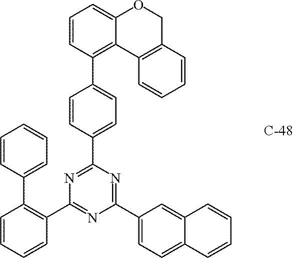 [SMILES: c1ccc(-c2ccccc2-c2nc(-c3ccc(-c4cccc5c4-c4ccccc4CO5)cc3)nc(-c3ccc4ccccc4c3)n2)cc1]

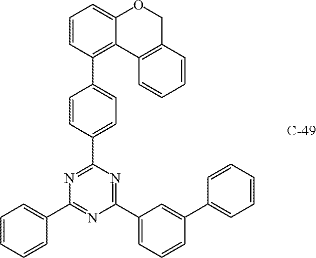 [SMILES: c1ccc(-c2cccc(-c3nc(-c4ccccc4)nc(-c4ccc(-c5cccc6c5-c5ccccc5CO6)cc4)n3)c2)cc1]

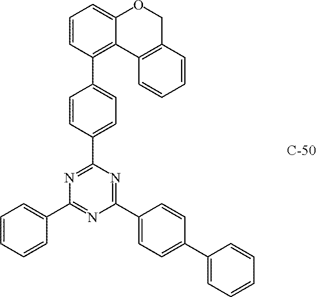 [SMILES: c1ccc(-c2ccc(-c3nc(-c4ccccc4)nc(-c4ccc(-c5cccc6c5-c5ccccc5CO6)cc4)n3)cc2)cc1]

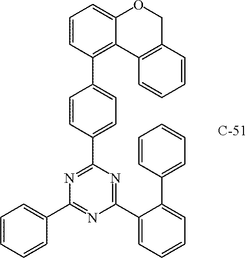 [SMILES: c1ccc(-c2nc(-c3ccc(-c4cccc5c4-c4ccccc4CO5)cc3)nc(-c3ccccc3-c3ccccc3)n2)cc1]

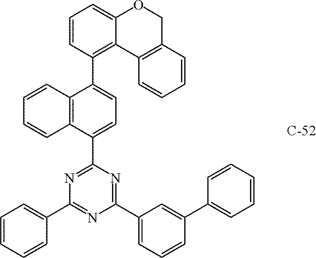 [SMILES: c1ccc(-c2cccc(-c3nc(-c4ccccc4)nc(-c4ccc(-c5cccc6c5-c5ccccc5CO6)c5ccccc45)n3)c2)cc1]

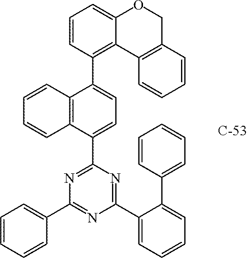 [SMILES: c1ccc(-c2nc(-c3ccccc3-c3ccccc3)nc(-c3ccc(-c4cccc5c4-c4ccccc4CO5)c4ccccc34)n2)cc1]

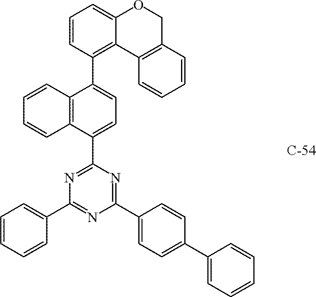 [SMILES: c1ccc(-c2ccc(-c3nc(-c4ccccc4)nc(-c4ccc(-c5cccc6c5-c5ccccc5CO6)c5ccccc45)n3)cc2)cc1]

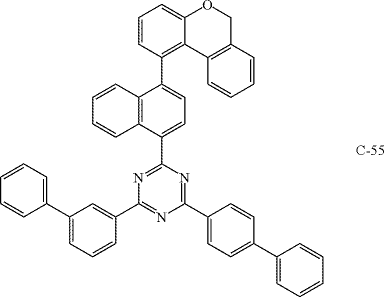 [SMILES: c1ccc(-c2ccc(-c3nc(-c4cccc(-c5ccccc5)c4)nc(-c4ccc(-c5cccc6c5-c5ccccc5CO6)c5ccccc45)n3)cc2)cc1]

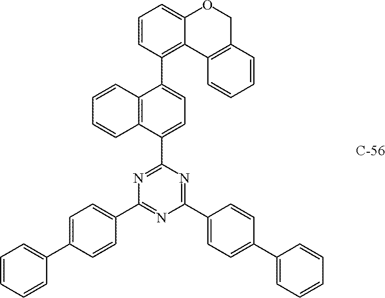 [SMILES: c1ccc(-c2ccc(-c3nc(-c4ccc(-c5ccccc5)cc4)nc(-c4ccc(-c5cccc6c5-c5ccccc5CO6)c5ccccc45)n3)cc2)cc1]

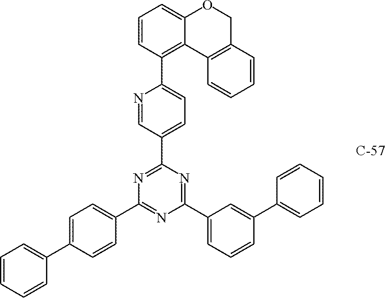 [SMILES: c1ccc(-c2ccc(-c3nc(-c4ccc(-c5cccc6c5-c5ccccc5CO6)nc4)nc(-c4cccc(-c5ccccc5)c4)n3)cc2)cc1]

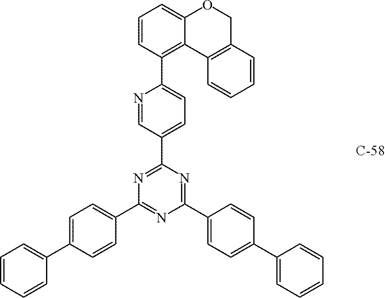 [SMILES: c1ccc(-c2ccc(-c3nc(-c4ccc(-c5ccccc5)cc4)nc(-c4ccc(-c5cccc6c5-c5ccccc5CO6)nc4)n3)cc2)cc1]

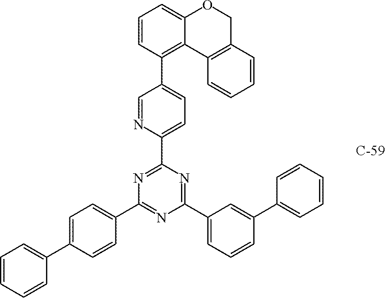 [SMILES: c1ccc(-c2ccc(-c3nc(-c4cccc(-c5ccccc5)c4)nc(-c4ccc(-c5cccc6c5-c5ccccc5CO6)cn4)n3)cc2)cc1]

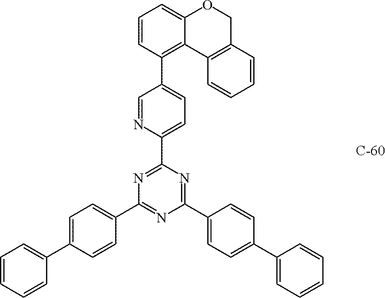 [SMILES: c1ccc(-c2ccc(-c3nc(-c4ccc(-c5ccccc5)cc4)nc(-c4ccc(-c5cccc6c5-c5ccccc5CO6)cn4)n3)cc2)cc1]

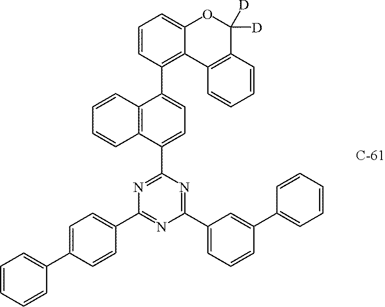 [SMILES: [2H]C1([2H])Oc2cccc(-c3ccc(-c4nc(-c5ccc(-c6ccccc6)cc5)nc(-c5cccc(-c6ccccc6)c5)n4)c4ccccc34)c2-c2ccccc21]

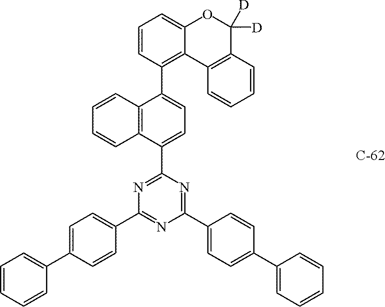 [SMILES: [2H]C1([2H])Oc2cccc(-c3ccc(-c4nc(-c5ccc(-c6ccccc6)cc5)nc(-c5ccc(-c6ccccc6)cc5)n4)c4ccccc34)c2-c2ccccc21]

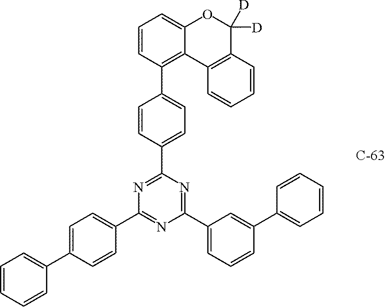 [SMILES: [2H]C1([2H])Oc2cccc(-c3ccc(-c4nc(-c5ccc(-c6ccccc6)cc5)nc(-c5cccc(-c6ccccc6)c5)n4)cc3)c2-c2ccccc21]

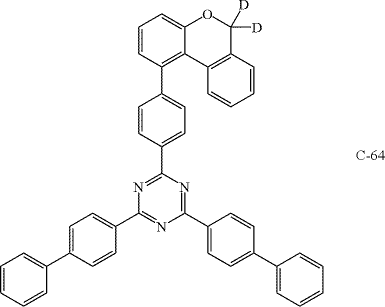 [SMILES: [2H]C1([2H])Oc2cccc(-c3ccc(-c4nc(-c5ccc(-c6ccccc6)cc5)nc(-c5ccc(-c6ccccc6)cc5)n4)cc3)c2-c2ccccc21]

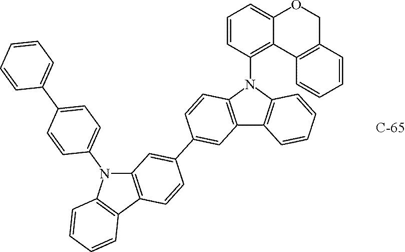 [SMILES: c1ccc(-c2ccc(-n3c4ccccc4c4ccc(-c5ccc6c(c5)c5ccccc5n6-c5cccc6c5-c5ccccc5CO6)cc43)cc2)cc1]

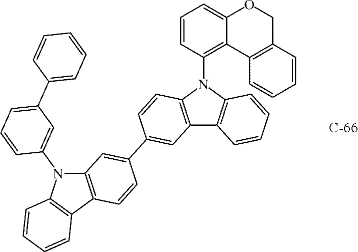 [SMILES: c1ccc(-c2cccc(-n3c4ccccc4c4ccc(-c5ccc6c(c5)c5ccccc5n6-c5cccc6c5-c5ccccc5CO6)cc43)c2)cc1]

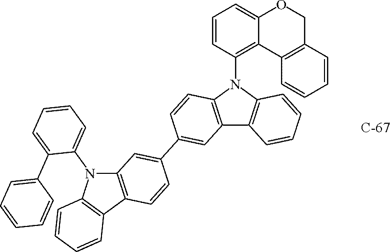 [SMILES: c1ccc(-c2ccccc2-n2c3ccccc3c3ccc(-c4ccc5c(c4)c4ccccc4n5-c4cccc5c4-c4ccccc4CO5)cc32)cc1]

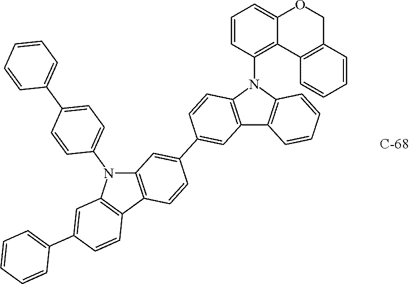 [SMILES: c1ccc(-c2ccc(-n3c4cc(-c5ccccc5)ccc4c4ccc(-c5ccc6c(c5)c5ccccc5n6-c5cccc6c5-c5ccccc5CO6)cc43)cc2)cc1]

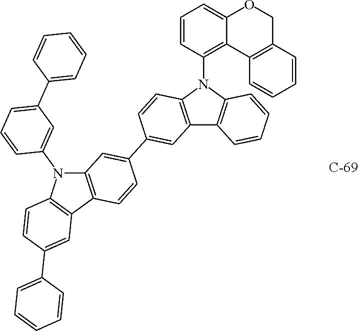 [SMILES: c1ccc(-c2cccc(-n3c4ccc(-c5ccccc5)cc4c4ccc(-c5ccc6c(c5)c5ccccc5n6-c5cccc6c5-c5ccccc5CO6)cc43)c2)cc1]

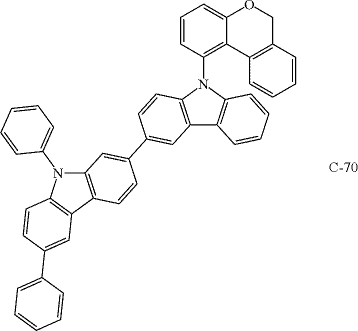 [SMILES: c1ccc(-c2ccc3c(c2)c2ccc(-c4ccc5c(c4)c4ccccc4n5-c4cccc5c4-c4ccccc4CO5)cc2n3-c2ccccc2)cc1]

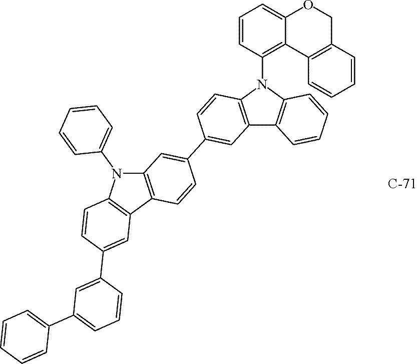 [SMILES: c1ccc(-c2cccc(-c3ccc4c(c3)c3ccc(-c5ccc6c(c5)c5ccccc5n6-c5cccc6c5-c5ccccc5CO6)cc3n4-c3ccccc3)c2)cc1]

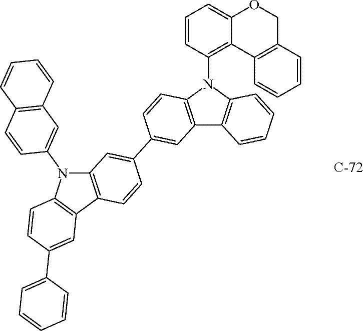 [SMILES: c1ccc(-c2ccc3c(c2)c2ccc(-c4ccc5c(c4)c4ccccc4n5-c4cccc5c4-c4ccccc4CO5)cc2n3-c2ccc3ccccc3c2)cc1]

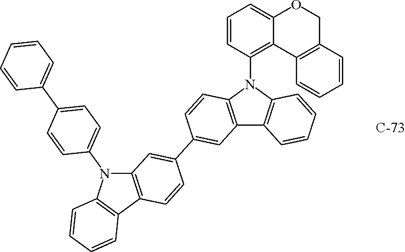 [SMILES: c1ccc(-c2ccc(-n3c4ccccc4c4ccc(-c5ccc6c(c5)c5ccccc5n6-c5cccc6c5-c5ccccc5CO6)cc43)cc2)cc1]

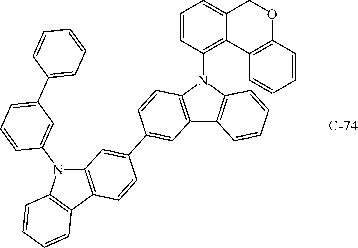 [SMILES: c1ccc(-c2cccc(-n3c4ccccc4c4ccc(-c5ccc6c(c5)c5ccccc5n6-c5cccc6c5-c5ccccc5OC6)cc43)c2)cc1]

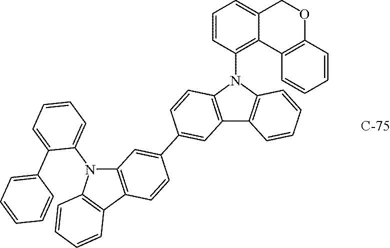 [SMILES: c1ccc(-c2ccccc2-n2c3ccccc3c3ccc(-c4ccc5c(c4)c4ccccc4n5-c4cccc5c4-c4ccccc4OC5)cc32)cc1]

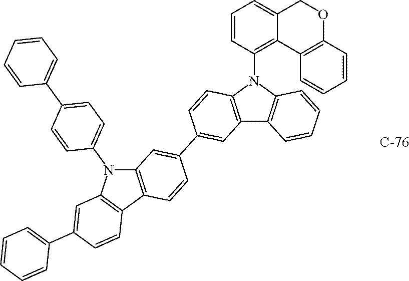 [SMILES: c1ccc(-c2ccc(-n3c4cc(-c5ccccc5)ccc4c4ccc(-c5ccc6c(c5)c5ccccc5n6-c5cccc6c5-c5ccccc5OC6)cc43)cc2)cc1]

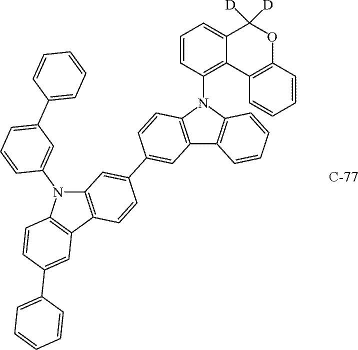 [SMILES: [2H]C1([2H])Oc2ccccc2-c2c(-n3c4ccccc4c4cc(-c5ccc6c7cc(-c8ccccc8)ccc7n(-c7cccc(-c8ccccc8)c7)c6c5)ccc43)cccc21]

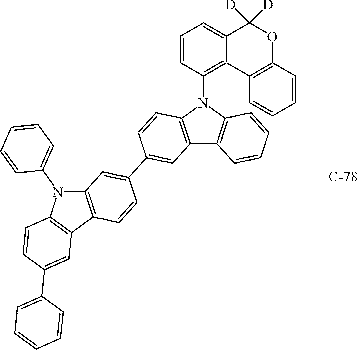 [SMILES: [2H]C1([2H])Oc2ccccc2-c2c(-n3c4ccccc4c4cc(-c5ccc6c7cc(-c8ccccc8)ccc7n(-c7ccccc7)c6c5)ccc43)cccc21]

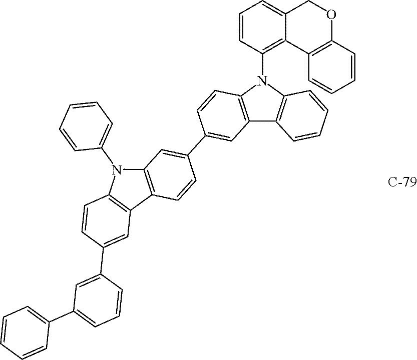 [SMILES: c1ccc(-c2cccc(-c3ccc4c(c3)c3ccc(-c5ccc6c(c5)c5ccccc5n6-c5cccc6c5-c5ccccc5OC6)cc3n4-c3ccccc3)c2)cc1]

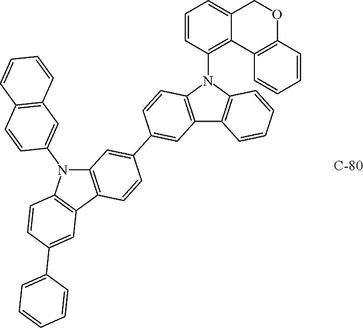 [SMILES: c1ccc(-c2ccc3c(c2)c2ccc(-c4ccc5c(c4)c4ccccc4n5-c4cccc5c4-c4ccccc4OC5)cc2n3-c2ccc3ccccc3c2)cc1]

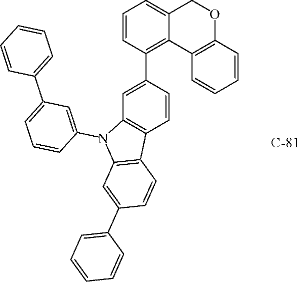 [SMILES: c1ccc(-c2cccc(-n3c4cc(-c5ccccc5)ccc4c4ccc(-c5cccc6c5-c5ccccc5OC6)cc43)c2)cc1]

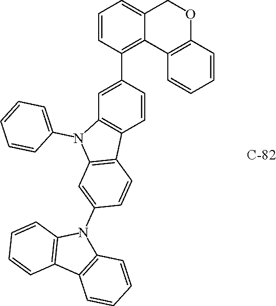 [SMILES: c1ccc(-n2c3cc(-c4cccc5c4-c4ccccc4OC5)ccc3c3ccc(-n4c5ccccc5c5ccccc54)cc32)cc1]

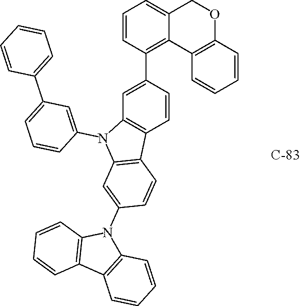 [SMILES: c1ccc(-c2cccc(-n3c4cc(-c5cccc6c5-c5ccccc5OC6)ccc4c4ccc(-n5c6ccccc6c6ccccc65)cc43)c2)cc1]

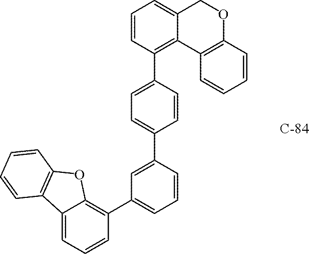 [SMILES: c1cc(-c2ccc(-c3cccc4c3-c3ccccc3OC4)cc2)cc(-c2cccc3c2oc2ccccc23)c1]

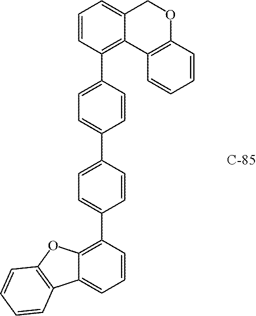 [SMILES: c1ccc2c(c1)OCc1cccc(-c3ccc(-c4ccc(-c5cccc6c5oc5ccccc56)cc4)cc3)c1-2]

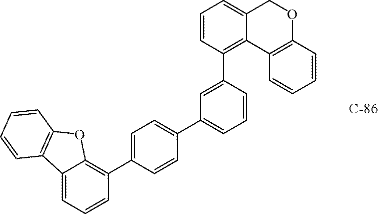 [SMILES: c1cc(-c2ccc(-c3cccc4c3oc3ccccc34)cc2)cc(-c2cccc3c2-c2ccccc2OC3)c1]

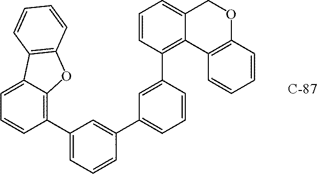 [SMILES: c1cc(-c2cccc(-c3cccc4c3oc3ccccc34)c2)cc(-c2cccc3c2-c2ccccc2OC3)c1]

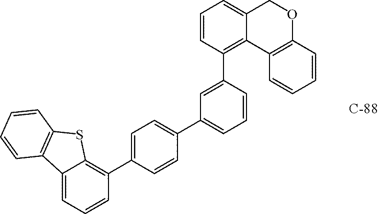 [SMILES: c1cc(-c2ccc(-c3cccc4c3sc3ccccc34)cc2)cc(-c2cccc3c2-c2ccccc2OC3)c1]

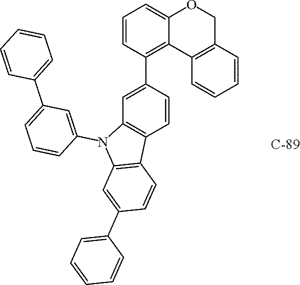 [SMILES: c1ccc(-c2cccc(-n3c4cc(-c5ccccc5)ccc4c4ccc(-c5cccc6c5-c5ccccc5CO6)cc43)c2)cc1]

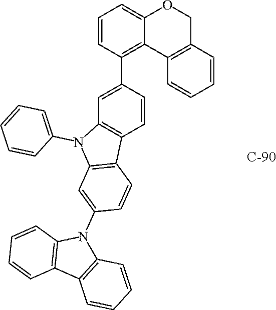 [SMILES: c1ccc(-n2c3cc(-c4cccc5c4-c4ccccc4CO5)ccc3c3ccc(-n4c5ccccc5c5ccccc54)cc32)cc1]

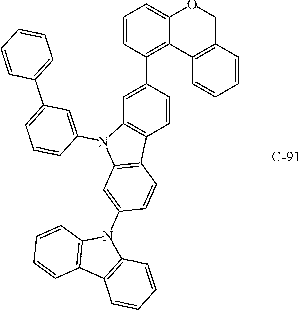 [SMILES: c1ccc(-c2cccc(-n3c4cc(-c5cccc6c5-c5ccccc5CO6)ccc4c4ccc(-n5c6ccccc6c6ccccc65)cc43)c2)cc1]

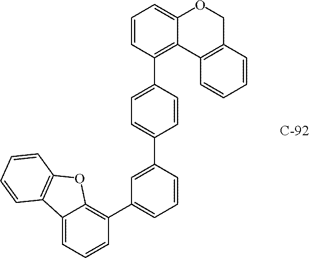 [SMILES: c1cc(-c2ccc(-c3cccc4c3-c3ccccc3CO4)cc2)cc(-c2cccc3c2oc2ccccc23)c1]

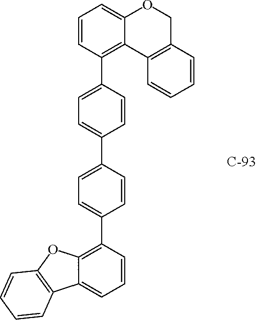 [SMILES: c1ccc2c(c1)COc1cccc(-c3ccc(-c4ccc(-c5cccc6c5oc5ccccc56)cc4)cc3)c1-2]

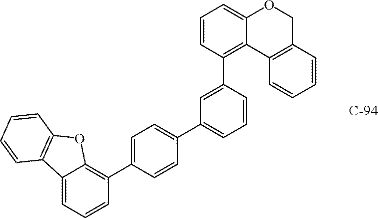 [SMILES: c1cc(-c2ccc(-c3cccc4c3oc3ccccc34)cc2)cc(-c2cccc3c2-c2ccccc2CO3)c1]

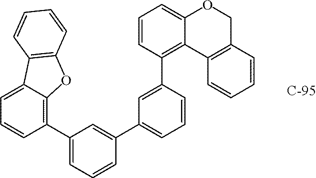 [SMILES: c1cc(-c2cccc(-c3cccc4c3oc3ccccc34)c2)cc(-c2cccc3c2-c2ccccc2CO3)c1]

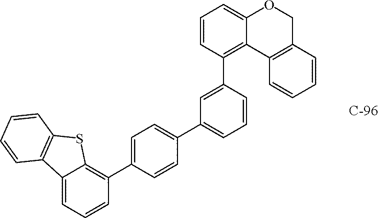 [SMILES: c1cc(-c2ccc(-c3cccc4c3sc3ccccc34)cc2)cc(-c2cccc3c2-c2ccccc2CO3)c1]

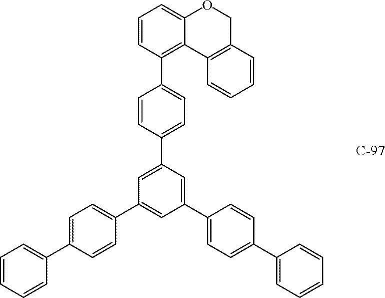 [SMILES: c1ccc(-c2ccc(-c3cc(-c4ccc(-c5ccccc5)cc4)cc(-c4ccc(-c5cccc6c5-c5ccccc5CO6)cc4)c3)cc2)cc1]

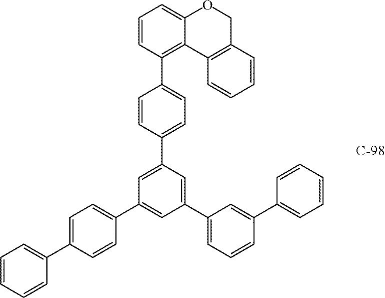 [SMILES: c1ccc(-c2ccc(-c3cc(-c4ccc(-c5cccc6c5-c5ccccc5CO6)cc4)cc(-c4cccc(-c5ccccc5)c4)c3)cc2)cc1]

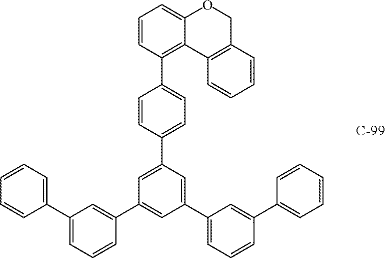 [SMILES: c1ccc(-c2cccc(-c3cc(-c4ccc(-c5cccc6c5-c5ccccc5CO6)cc4)cc(-c4cccc(-c5ccccc5)c4)c3)c2)cc1]

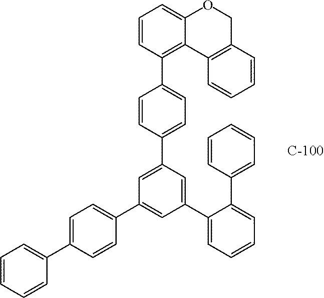 [SMILES: c1ccc(-c2ccc(-c3cc(-c4ccc(-c5cccc6c5-c5ccccc5CO6)cc4)cc(-c4ccccc4-c4ccccc4)c3)cc2)cc1]

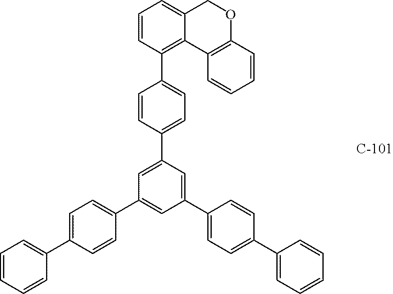 [SMILES: c1ccc(-c2ccc(-c3cc(-c4ccc(-c5ccccc5)cc4)cc(-c4ccc(-c5cccc6c5-c5ccccc5OC6)cc4)c3)cc2)cc1]

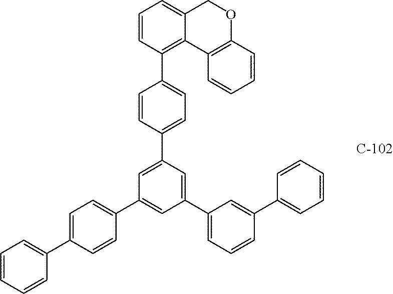 [SMILES: c1ccc(-c2ccc(-c3cc(-c4ccc(-c5cccc6c5-c5ccccc5OC6)cc4)cc(-c4cccc(-c5ccccc5)c4)c3)cc2)cc1]

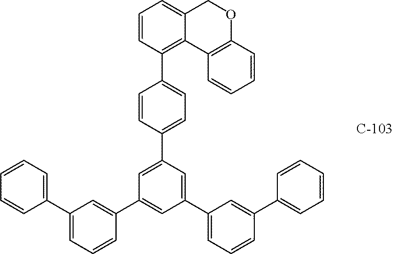 [SMILES: c1ccc(-c2cccc(-c3cc(-c4ccc(-c5cccc6c5-c5ccccc5OC6)cc4)cc(-c4cccc(-c5ccccc5)c4)c3)c2)cc1]

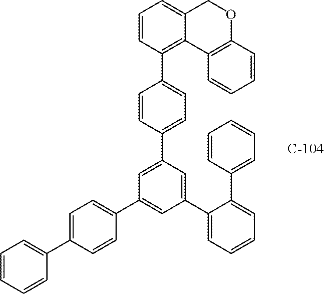 [SMILES: c1ccc(-c2ccc(-c3cc(-c4ccc(-c5cccc6c5-c5ccccc5OC6)cc4)cc(-c4ccccc4-c4ccccc4)c3)cc2)cc1]

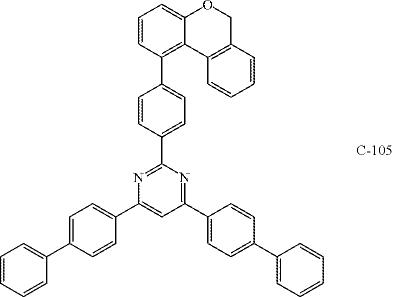 [SMILES: c1ccc(-c2ccc(-c3cc(-c4ccc(-c5ccccc5)cc4)nc(-c4ccc(-c5cccc6c5-c5ccccc5CO6)cc4)n3)cc2)cc1]

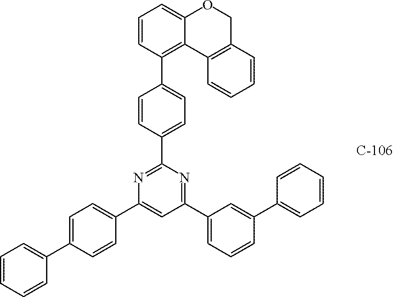 [SMILES: c1ccc(-c2ccc(-c3cc(-c4cccc(-c5ccccc5)c4)nc(-c4ccc(-c5cccc6c5-c5ccccc5CO6)cc4)n3)cc2)cc1]

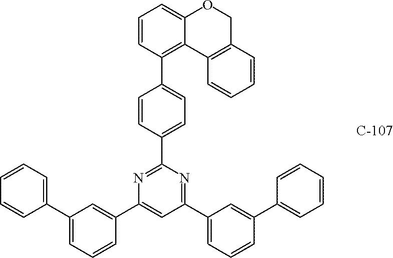 [SMILES: c1ccc(-c2cccc(-c3cc(-c4cccc(-c5ccccc5)c4)nc(-c4ccc(-c5cccc6c5-c5ccccc5CO6)cc4)n3)c2)cc1]

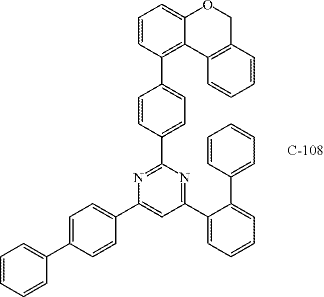 [SMILES: c1ccc(-c2ccc(-c3cc(-c4ccccc4-c4ccccc4)nc(-c4ccc(-c5cccc6c5-c5ccccc5CO6)cc4)n3)cc2)cc1]

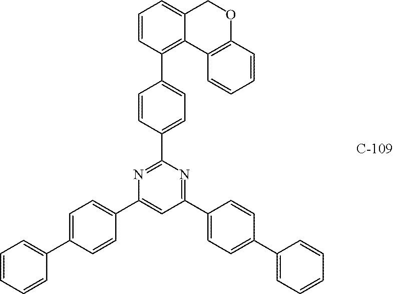 [SMILES: c1ccc(-c2ccc(-c3cc(-c4ccc(-c5ccccc5)cc4)nc(-c4ccc(-c5cccc6c5-c5ccccc5OC6)cc4)n3)cc2)cc1]

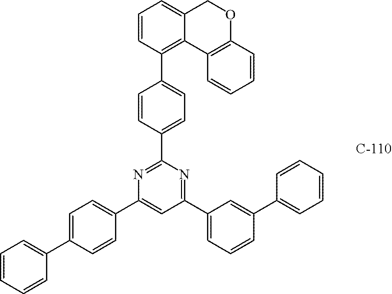 [SMILES: c1ccc(-c2ccc(-c3cc(-c4cccc(-c5ccccc5)c4)nc(-c4ccc(-c5cccc6c5-c5ccccc5OC6)cc4)n3)cc2)cc1]

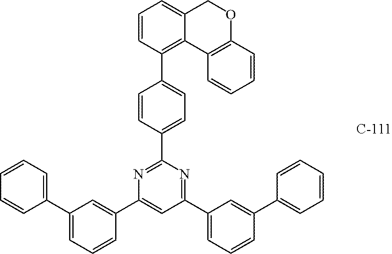 [SMILES: c1ccc(-c2cccc(-c3cc(-c4cccc(-c5ccccc5)c4)nc(-c4ccc(-c5cccc6c5-c5ccccc5OC6)cc4)n3)c2)cc1]

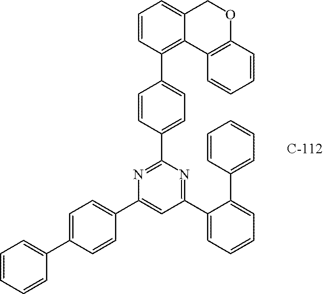 [SMILES: c1ccc(-c2ccc(-c3cc(-c4ccccc4-c4ccccc4)nc(-c4ccc(-c5cccc6c5-c5ccccc5OC6)cc4)n3)cc2)cc1]

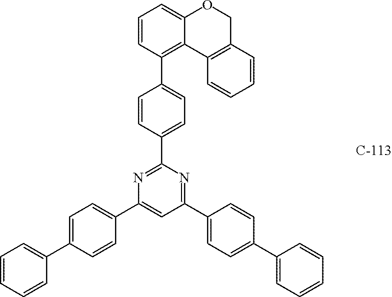 [SMILES: c1ccc(-c2ccc(-c3cc(-c4ccc(-c5ccccc5)cc4)nc(-c4ccc(-c5cccc6c5-c5ccccc5CO6)cc4)n3)cc2)cc1]

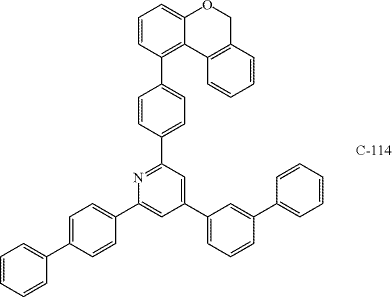 [SMILES: c1ccc(-c2ccc(-c3cc(-c4cccc(-c5ccccc5)c4)cc(-c4ccc(-c5cccc6c5-c5ccccc5CO6)cc4)n3)cc2)cc1]

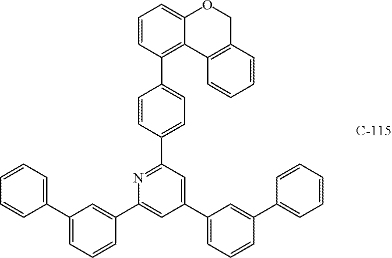 [SMILES: c1ccc(-c2cccc(-c3cc(-c4ccc(-c5cccc6c5-c5ccccc5CO6)cc4)nc(-c4cccc(-c5ccccc5)c4)c3)c2)cc1]

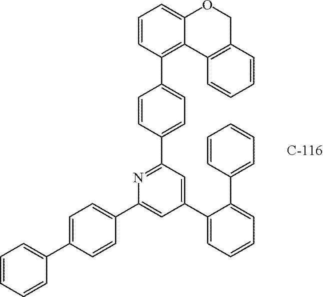 [SMILES: c1ccc(-c2ccc(-c3cc(-c4ccccc4-c4ccccc4)cc(-c4ccc(-c5cccc6c5-c5ccccc5CO6)cc4)n3)cc2)cc1]

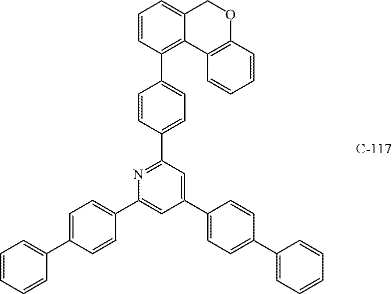 [SMILES: c1ccc(-c2ccc(-c3cc(-c4ccc(-c5ccccc5)cc4)nc(-c4ccc(-c5cccc6c5-c5ccccc5OC6)cc4)c3)cc2)cc1]

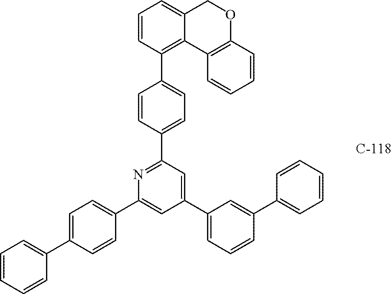 [SMILES: c1ccc(-c2ccc(-c3cc(-c4cccc(-c5ccccc5)c4)cc(-c4ccc(-c5cccc6c5-c5ccccc5OC6)cc4)n3)cc2)cc1]

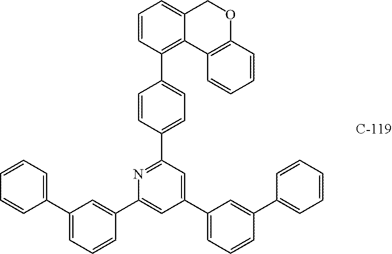 [SMILES: c1ccc(-c2cccc(-c3cc(-c4ccc(-c5cccc6c5-c5ccccc5OC6)cc4)nc(-c4cccc(-c5ccccc5)c4)c3)c2)cc1]

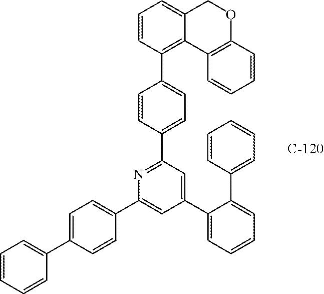 [SMILES: c1ccc(-c2ccc(-c3cc(-c4ccccc4-c4ccccc4)cc(-c4ccc(-c5cccc6c5-c5ccccc5OC6)cc4)n3)cc2)cc1]

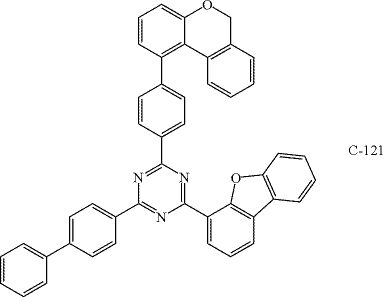 [SMILES: c1ccc(-c2ccc(-c3nc(-c4ccc(-c5cccc6c5-c5ccccc5CO6)cc4)nc(-c4cccc5c4oc4ccccc45)n3)cc2)cc1]

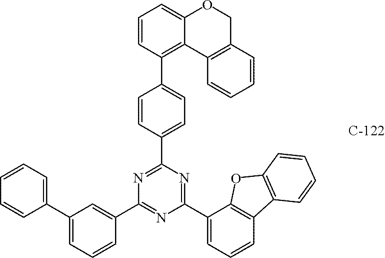 [SMILES: c1ccc(-c2cccc(-c3nc(-c4ccc(-c5cccc6c5-c5ccccc5CO6)cc4)nc(-c4cccc5c4oc4ccccc45)n3)c2)cc1]

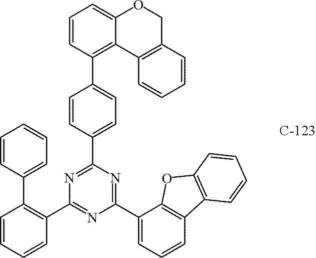 [SMILES: c1ccc(-c2ccccc2-c2nc(-c3ccc(-c4cccc5c4-c4ccccc4CO5)cc3)nc(-c3cccc4c3oc3ccccc34)n2)cc1]

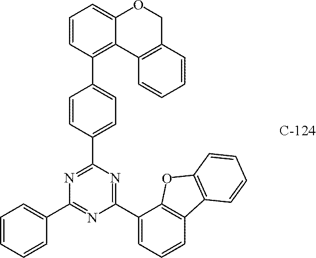 [SMILES: c1ccc(-c2nc(-c3ccc(-c4cccc5c4-c4ccccc4CO5)cc3)nc(-c3cccc4c3oc3ccccc34)n2)cc1]

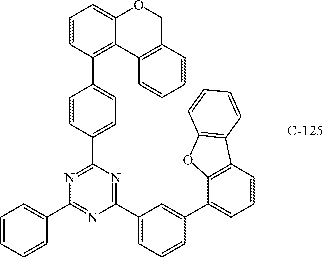 [SMILES: c1ccc(-c2nc(-c3ccc(-c4cccc5c4-c4ccccc4CO5)cc3)nc(-c3cccc(-c4cccc5c4oc4ccccc45)c3)n2)cc1]

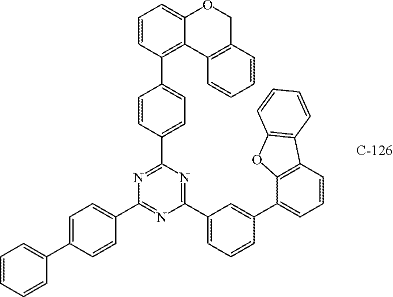 [SMILES: c1ccc(-c2ccc(-c3nc(-c4ccc(-c5cccc6c5-c5ccccc5CO6)cc4)nc(-c4cccc(-c5cccc6c5oc5ccccc56)c4)n3)cc2)cc1]

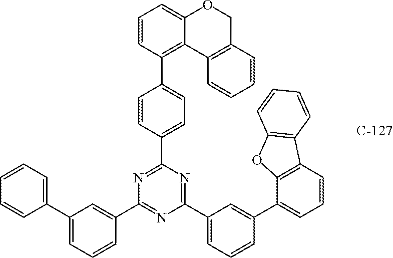 [SMILES: c1ccc(-c2cccc(-c3nc(-c4ccc(-c5cccc6c5-c5ccccc5CO6)cc4)nc(-c4cccc(-c5cccc6c5oc5ccccc56)c4)n3)c2)cc1]

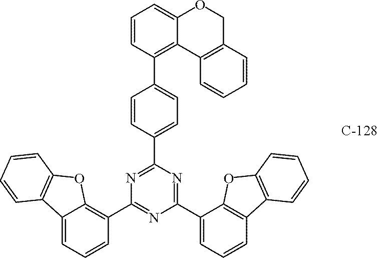 [SMILES: c1ccc2c(c1)COc1cccc(-c3ccc(-c4nc(-c5cccc6c5oc5ccccc56)nc(-c5cccc6c5oc5ccccc56)n4)cc3)c1-2]

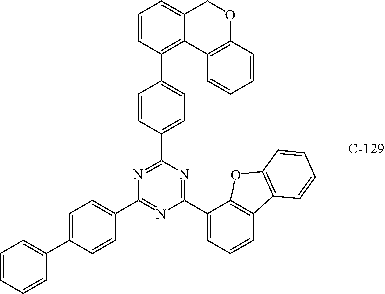 [SMILES: c1ccc(-c2ccc(-c3nc(-c4ccc(-c5cccc6c5-c5ccccc5OC6)cc4)nc(-c4cccc5c4oc4ccccc45)n3)cc2)cc1]

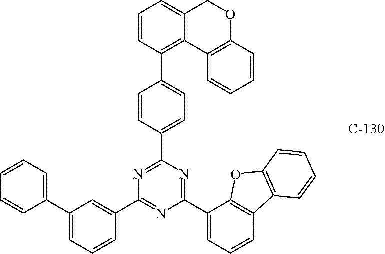 [SMILES: c1ccc(-c2cccc(-c3nc(-c4ccc(-c5cccc6c5-c5ccccc5OC6)cc4)nc(-c4cccc5c4oc4ccccc45)n3)c2)cc1]

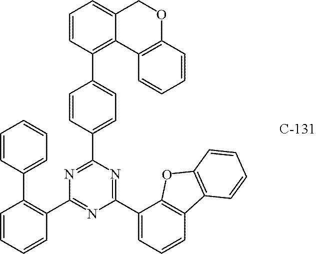 [SMILES: c1ccc(-c2ccccc2-c2nc(-c3ccc(-c4cccc5c4-c4ccccc4OC5)cc3)nc(-c3cccc4c3oc3ccccc34)n2)cc1]

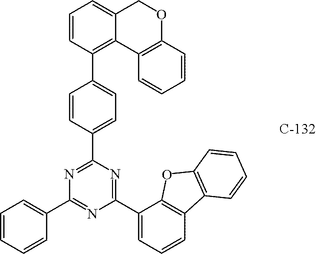 [SMILES: c1ccc(-c2nc(-c3ccc(-c4cccc5c4-c4ccccc4OC5)cc3)nc(-c3cccc4c3oc3ccccc34)n2)cc1]

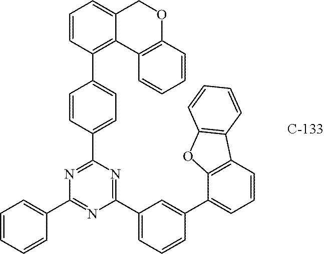 [SMILES: c1ccc(-c2nc(-c3ccc(-c4cccc5c4-c4ccccc4OC5)cc3)nc(-c3cccc(-c4cccc5c4oc4ccccc45)c3)n2)cc1]

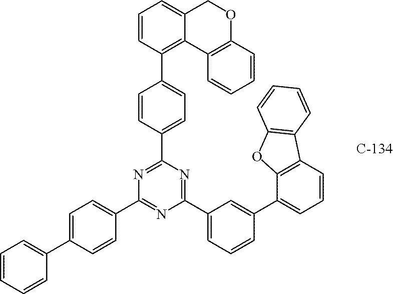 [SMILES: c1ccc(-c2ccc(-c3nc(-c4ccc(-c5cccc6c5-c5ccccc5OC6)cc4)nc(-c4cccc(-c5cccc6c5oc5ccccc56)c4)n3)cc2)cc1]

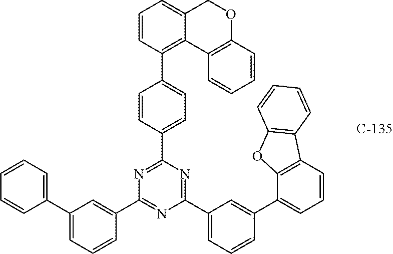 [SMILES: c1ccc(-c2cccc(-c3nc(-c4ccc(-c5cccc6c5-c5ccccc5OC6)cc4)nc(-c4cccc(-c5cccc6c5oc5ccccc56)c4)n3)c2)cc1]

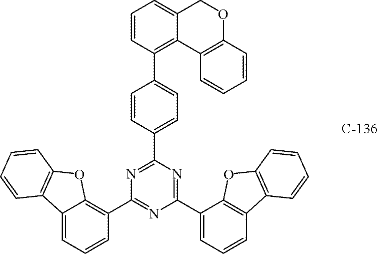 [SMILES: c1ccc2c(c1)OCc1cccc(-c3ccc(-c4nc(-c5cccc6c5oc5ccccc56)nc(-c5cccc6c5oc5ccccc56)n4)cc3)c1-2]

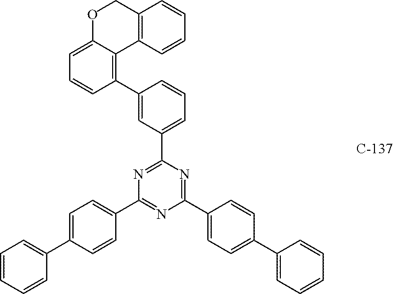 [SMILES: c1ccc(-c2ccc(-c3nc(-c4ccc(-c5ccccc5)cc4)nc(-c4cccc(-c5cccc6c5-c5ccccc5CO6)c4)n3)cc2)cc1]

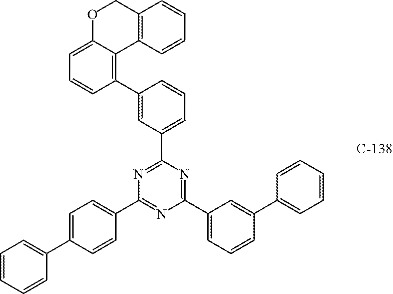 [SMILES: c1ccc(-c2ccc(-c3nc(-c4cccc(-c5ccccc5)c4)nc(-c4cccc(-c5cccc6c5-c5ccccc5CO6)c4)n3)cc2)cc1]

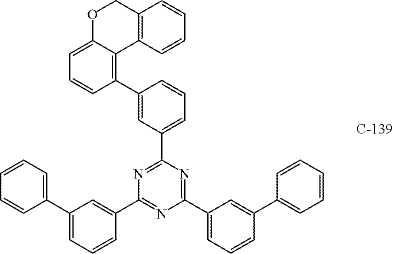 [SMILES: c1ccc(-c2cccc(-c3nc(-c4cccc(-c5ccccc5)c4)nc(-c4cccc(-c5cccc6c5-c5ccccc5CO6)c4)n3)c2)cc1]

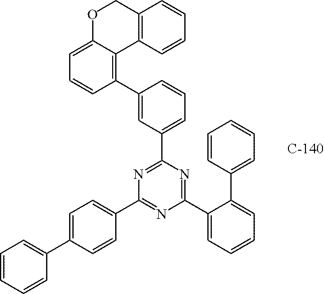 [SMILES: c1ccc(-c2ccc(-c3nc(-c4cccc(-c5cccc6c5-c5ccccc5CO6)c4)nc(-c4ccccc4-c4ccccc4)n3)cc2)cc1]

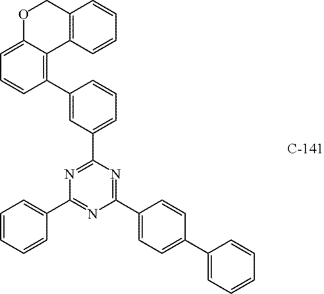 [SMILES: c1ccc(-c2ccc(-c3nc(-c4ccccc4)nc(-c4cccc(-c5cccc6c5-c5ccccc5CO6)c4)n3)cc2)cc1]

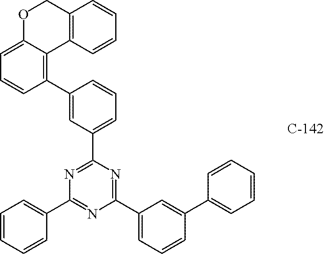 [SMILES: c1ccc(-c2cccc(-c3nc(-c4ccccc4)nc(-c4cccc(-c5cccc6c5-c5ccccc5CO6)c4)n3)c2)cc1]

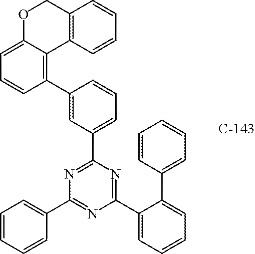 [SMILES: c1ccc(-c2nc(-c3cccc(-c4cccc5c4-c4ccccc4CO5)c3)nc(-c3ccccc3-c3ccccc3)n2)cc1]

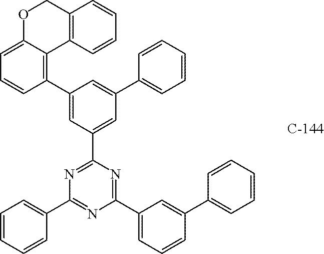 [SMILES: c1ccc(-c2cccc(-c3nc(-c4ccccc4)nc(-c4cc(-c5ccccc5)cc(-c5cccc6c5-c5ccccc5CO6)c4)n3)c2)cc1]

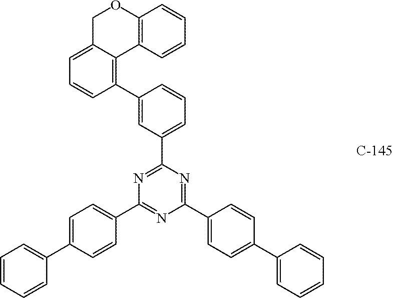 [SMILES: c1ccc(-c2ccc(-c3nc(-c4ccc(-c5ccccc5)cc4)nc(-c4cccc(-c5cccc6c5-c5ccccc5OC6)c4)n3)cc2)cc1]

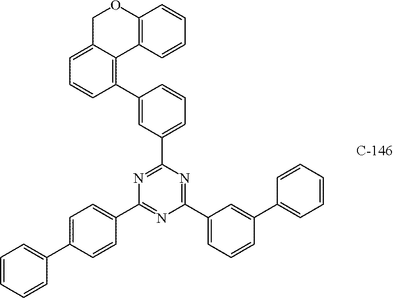 [SMILES: c1ccc(-c2ccc(-c3nc(-c4cccc(-c5ccccc5)c4)nc(-c4cccc(-c5cccc6c5-c5ccccc5OC6)c4)n3)cc2)cc1]

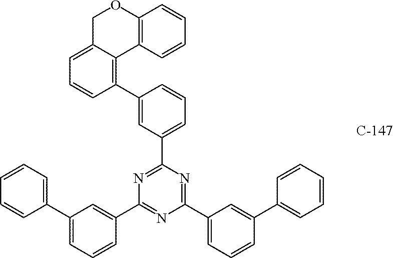 [SMILES: c1ccc(-c2cccc(-c3nc(-c4cccc(-c5ccccc5)c4)nc(-c4cccc(-c5cccc6c5-c5ccccc5OC6)c4)n3)c2)cc1]

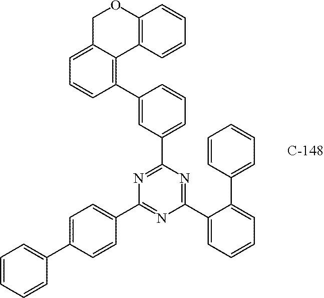 [SMILES: c1ccc(-c2ccc(-c3nc(-c4cccc(-c5cccc6c5-c5ccccc5OC6)c4)nc(-c4ccccc4-c4ccccc4)n3)cc2)cc1]

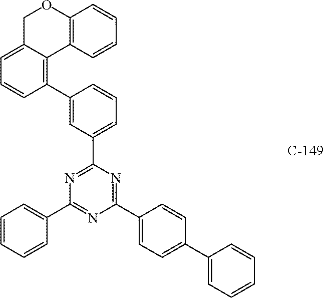 [SMILES: c1ccc(-c2ccc(-c3nc(-c4ccccc4)nc(-c4cccc(-c5cccc6c5-c5ccccc5OC6)c4)n3)cc2)cc1]

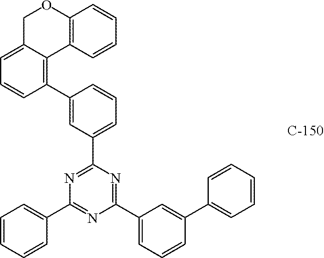 [SMILES: c1ccc(-c2cccc(-c3nc(-c4ccccc4)nc(-c4cccc(-c5cccc6c5-c5ccccc5OC6)c4)n3)c2)cc1]

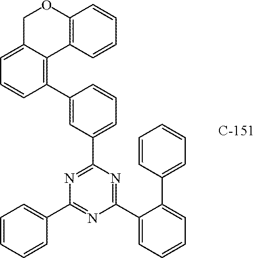 [SMILES: c1ccc(-c2nc(-c3cccc(-c4cccc5c4-c4ccccc4OC5)c3)nc(-c3ccccc3-c3ccccc3)n2)cc1]

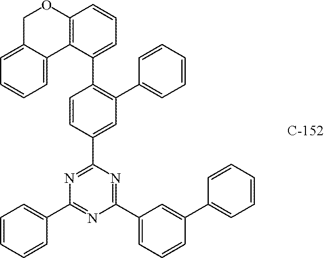 [SMILES: c1ccc(-c2cccc(-c3nc(-c4ccccc4)nc(-c4ccc(-c5cccc6c5-c5ccccc5CO6)c(-c5ccccc5)c4)n3)c2)cc1]

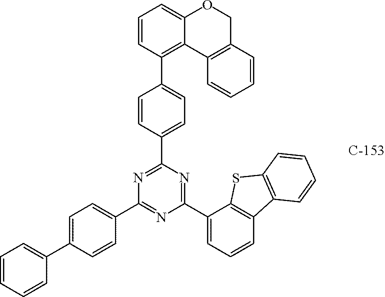 [SMILES: c1ccc(-c2ccc(-c3nc(-c4ccc(-c5cccc6c5-c5ccccc5CO6)cc4)nc(-c4cccc5c4sc4ccccc45)n3)cc2)cc1]

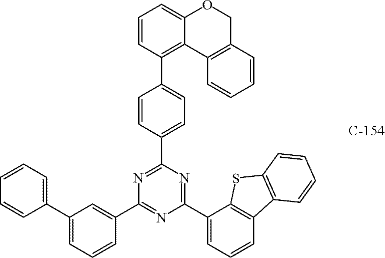 [SMILES: c1ccc(-c2cccc(-c3nc(-c4ccc(-c5cccc6c5-c5ccccc5CO6)cc4)nc(-c4cccc5c4sc4ccccc45)n3)c2)cc1]

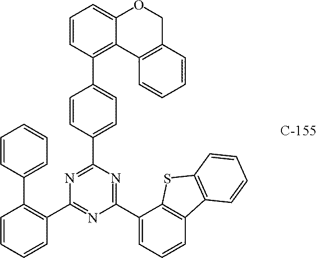 [SMILES: c1ccc(-c2ccccc2-c2nc(-c3ccc(-c4cccc5c4-c4ccccc4CO5)cc3)nc(-c3cccc4c3sc3ccccc34)n2)cc1]

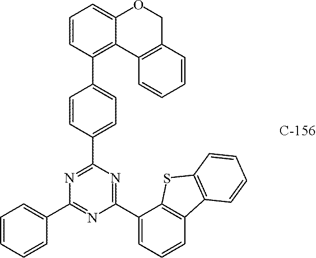 [SMILES: c1ccc(-c2nc(-c3ccc(-c4cccc5c4-c4ccccc4CO5)cc3)nc(-c3cccc4c3sc3ccccc34)n2)cc1]

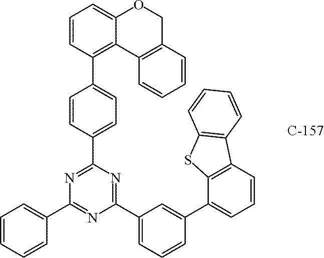 [SMILES: c1ccc(-c2nc(-c3ccc(-c4cccc5c4-c4ccccc4CO5)cc3)nc(-c3cccc(-c4cccc5c4sc4ccccc45)c3)n2)cc1]

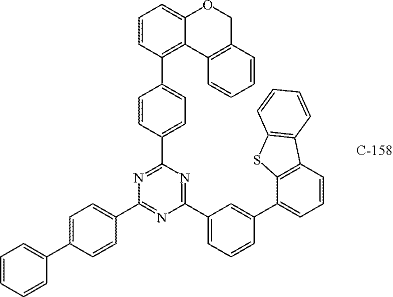 [SMILES: c1ccc(-c2ccc(-c3nc(-c4ccc(-c5cccc6c5-c5ccccc5CO6)cc4)nc(-c4cccc(-c5cccc6c5sc5ccccc56)c4)n3)cc2)cc1]

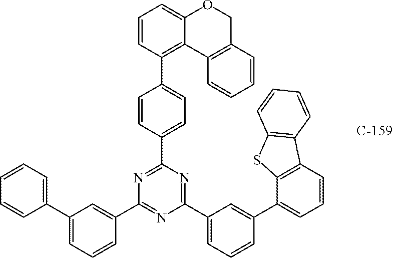 [SMILES: c1ccc(-c2cccc(-c3nc(-c4ccc(-c5cccc6c5-c5ccccc5CO6)cc4)nc(-c4cccc(-c5cccc6c5sc5ccccc56)c4)n3)c2)cc1]

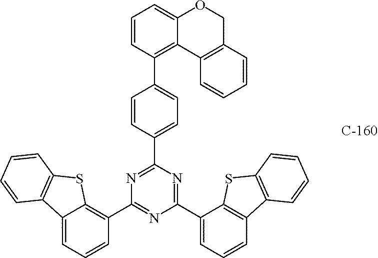 [SMILES: c1ccc2c(c1)COc1cccc(-c3ccc(-c4nc(-c5cccc6c5sc5ccccc56)nc(-c5cccc6c5sc5ccccc56)n4)cc3)c1-2]

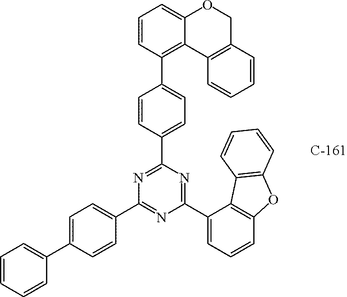 [SMILES: c1ccc(-c2ccc(-c3nc(-c4ccc(-c5cccc6c5-c5ccccc5CO6)cc4)nc(-c4cccc5oc6ccccc6c45)n3)cc2)cc1]

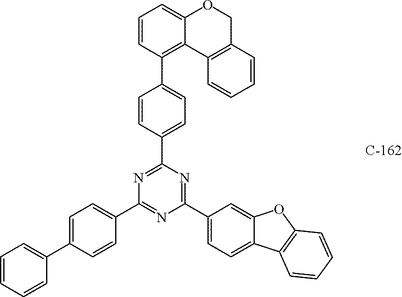 [SMILES: c1ccc(-c2ccc(-c3nc(-c4ccc(-c5cccc6c5-c5ccccc5CO6)cc4)nc(-c4ccc5c(c4)oc4ccccc45)n3)cc2)cc1]

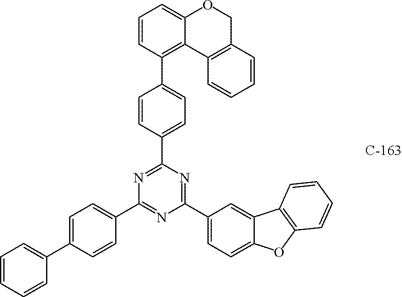 [SMILES: c1ccc(-c2ccc(-c3nc(-c4ccc(-c5cccc6c5-c5ccccc5CO6)cc4)nc(-c4ccc5oc6ccccc6c5c4)n3)cc2)cc1]

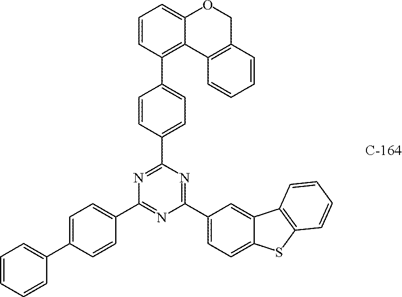 [SMILES: c1ccc(-c2ccc(-c3nc(-c4ccc(-c5cccc6c5-c5ccccc5CO6)cc4)nc(-c4ccc5sc6ccccc6c5c4)n3)cc2)cc1]

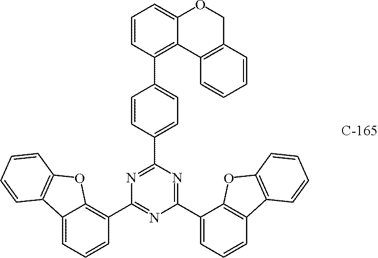 [SMILES: c1ccc2c(c1)COc1cccc(-c3ccc(-c4nc(-c5cccc6c5oc5ccccc56)nc(-c5cccc6c5oc5ccccc56)n4)cc3)c1-2]

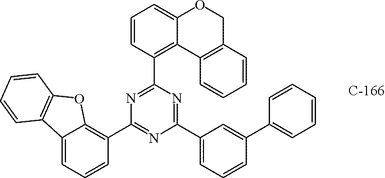 [SMILES: c1ccc(-c2cccc(-c3nc(-c4cccc5c4-c4ccccc4CO5)nc(-c4cccc5c4oc4ccccc45)n3)c2)cc1]

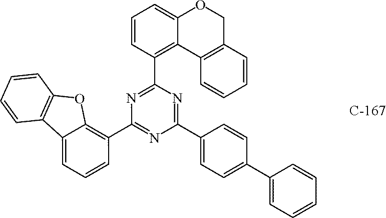 [SMILES: c1ccc(-c2ccc(-c3nc(-c4cccc5c4-c4ccccc4CO5)nc(-c4cccc5c4oc4ccccc45)n3)cc2)cc1]

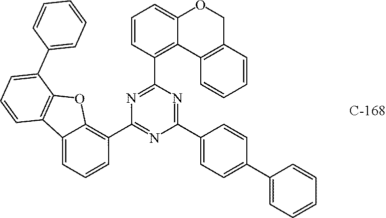 [SMILES: c1ccc(-c2ccc(-c3nc(-c4cccc5c4-c4ccccc4CO5)nc(-c4cccc5c4oc4c(-c6ccccc6)cccc45)n3)cc2)cc1]

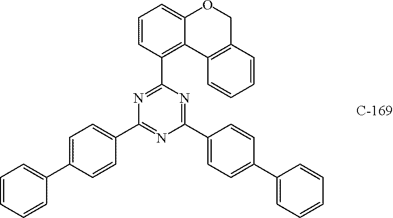 [SMILES: c1ccc(-c2ccc(-c3nc(-c4ccc(-c5ccccc5)cc4)nc(-c4cccc5c4-c4ccccc4CO5)n3)cc2)cc1]

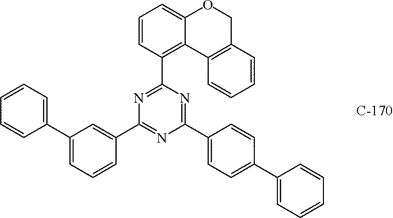 [SMILES: c1ccc(-c2ccc(-c3nc(-c4cccc(-c5ccccc5)c4)nc(-c4cccc5c4-c4ccccc4CO5)n3)cc2)cc1]

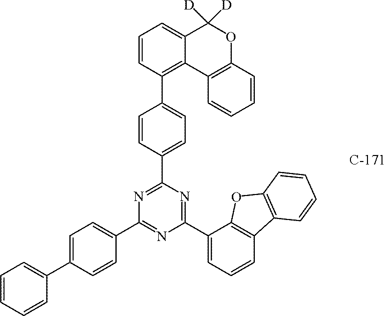 [SMILES: [2H]C1([2H])Oc2ccccc2-c2c(-c3ccc(-c4nc(-c5ccc(-c6ccccc6)cc5)nc(-c5cccc6c5oc5ccccc56)n4)cc3)cccc21]

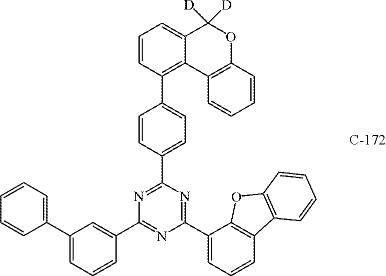 [SMILES: [2H]C1([2H])Oc2ccccc2-c2c(-c3ccc(-c4nc(-c5cccc(-c6ccccc6)c5)nc(-c5cccc6c5oc5ccccc56)n4)cc3)cccc21]

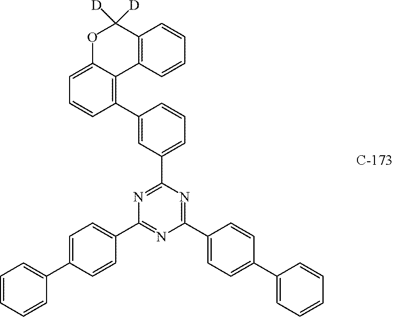 [SMILES: [2H]C1([2H])Oc2cccc(-c3cccc(-c4nc(-c5ccc(-c6ccccc6)cc5)nc(-c5ccc(-c6ccccc6)cc5)n4)c3)c2-c2ccccc21]

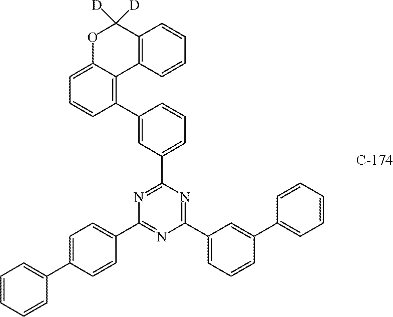 [SMILES: [2H]C1([2H])Oc2cccc(-c3cccc(-c4nc(-c5ccc(-c6ccccc6)cc5)nc(-c5cccc(-c6ccccc6)c5)n4)c3)c2-c2ccccc21]

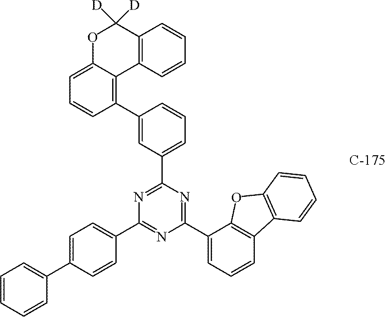 [SMILES: [2H]C1([2H])Oc2cccc(-c3cccc(-c4nc(-c5ccc(-c6ccccc6)cc5)nc(-c5cccc6c5oc5ccccc56)n4)c3)c2-c2ccccc21]

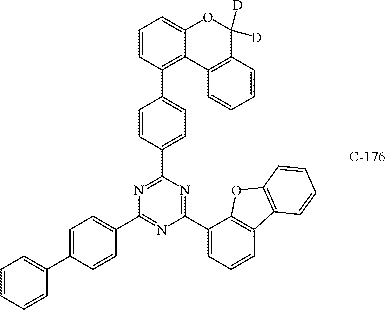 [SMILES: [2H]C1([2H])Oc2cccc(-c3ccc(-c4nc(-c5ccc(-c6ccccc6)cc5)nc(-c5cccc6c5oc5ccccc56)n4)cc3)c2-c2ccccc21]

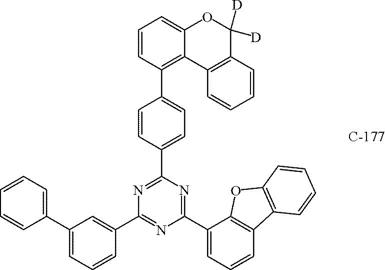 [SMILES: [2H]C1([2H])Oc2cccc(-c3ccc(-c4nc(-c5cccc(-c6ccccc6)c5)nc(-c5cccc6c5oc5ccccc56)n4)cc3)c2-c2ccccc21]

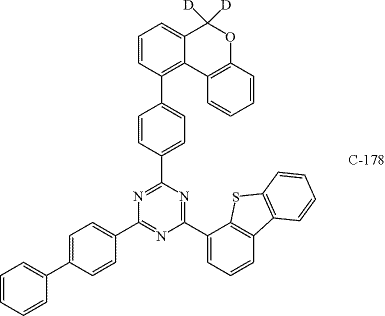 [SMILES: [2H]C1([2H])Oc2ccccc2-c2c(-c3ccc(-c4nc(-c5ccc(-c6ccccc6)cc5)nc(-c5cccc6c5sc5ccccc56)n4)cc3)cccc21]

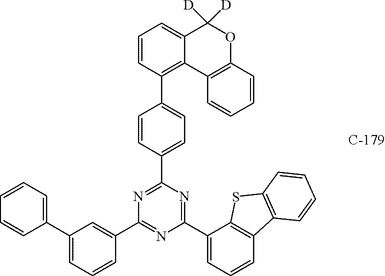 [SMILES: [2H]C1([2H])Oc2ccccc2-c2c(-c3ccc(-c4nc(-c5cccc(-c6ccccc6)c5)nc(-c5cccc6c5sc5ccccc56)n4)cc3)cccc21]

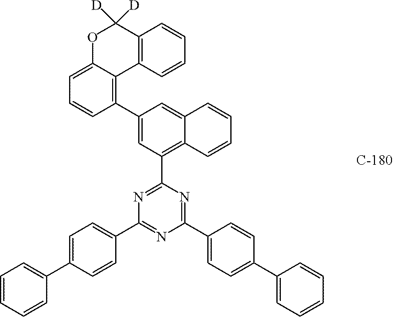 [SMILES: [2H]C1([2H])Oc2cccc(-c3cc(-c4nc(-c5ccc(-c6ccccc6)cc5)nc(-c5ccc(-c6ccccc6)cc5)n4)c4ccccc4c3)c2-c2ccccc21]

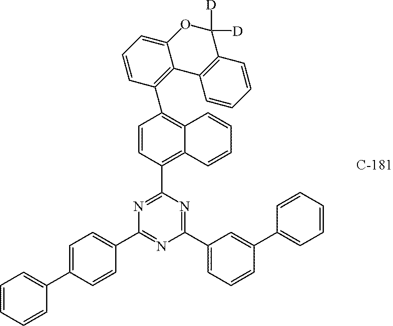 [SMILES: [2H]C1([2H])Oc2cccc(-c3ccc(-c4nc(-c5ccc(-c6ccccc6)cc5)nc(-c5cccc(-c6ccccc6)c5)n4)c4ccccc34)c2-c2ccccc21]

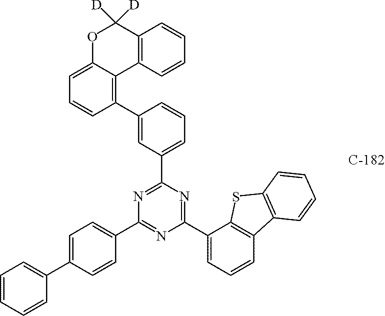 [SMILES: [2H]C1([2H])Oc2cccc(-c3cccc(-c4nc(-c5ccc(-c6ccccc6)cc5)nc(-c5cccc6c5sc5ccccc56)n4)c3)c2-c2ccccc21]

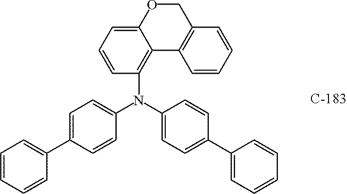 [SMILES: c1ccc(-c2ccc(N(c3ccc(-c4ccccc4)cc3)c3cccc4c3-c3ccccc3CO4)cc2)cc1]

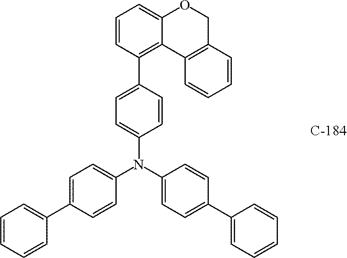 [SMILES: c1ccc(-c2ccc(N(c3ccc(-c4ccccc4)cc3)c3ccc(-c4cccc5c4-c4ccccc4CO5)cc3)cc2)cc1]

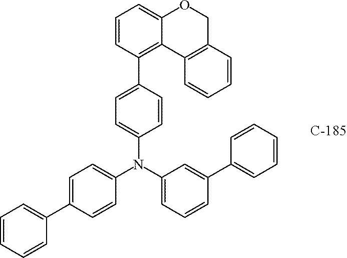 [SMILES: c1ccc(-c2ccc(N(c3ccc(-c4cccc5c4-c4ccccc4CO5)cc3)c3cccc(-c4ccccc4)c3)cc2)cc1]

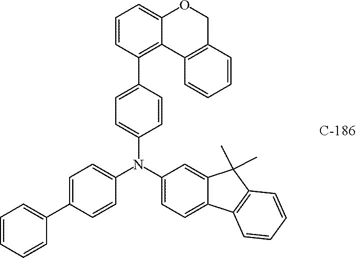 [SMILES: CC1(C)c2ccccc2-c2ccc(N(c3ccc(-c4ccccc4)cc3)c3ccc(-c4cccc5c4-c4ccccc4CO5)cc3)cc21]

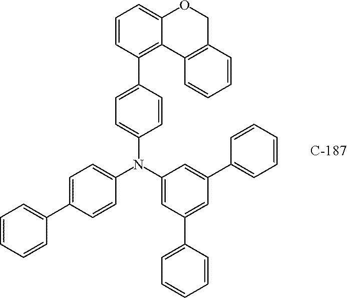 [SMILES: c1ccc(-c2ccc(N(c3ccc(-c4cccc5c4-c4ccccc4CO5)cc3)c3cc(-c4ccccc4)cc(-c4ccccc4)c3)cc2)cc1]

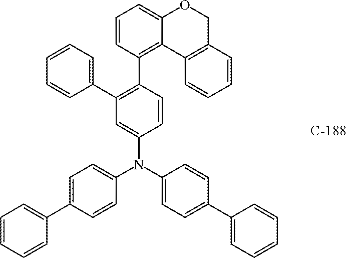 [SMILES: c1ccc(-c2ccc(N(c3ccc(-c4ccccc4)cc3)c3ccc(-c4cccc5c4-c4ccccc4CO5)c(-c4ccccc4)c3)cc2)cc1]

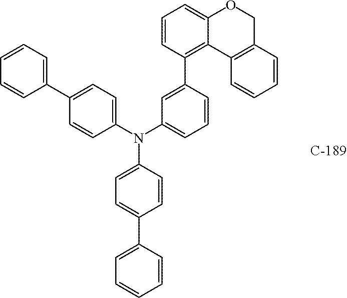 [SMILES: c1ccc(-c2ccc(N(c3ccc(-c4ccccc4)cc3)c3cccc(-c4cccc5c4-c4ccccc4CO5)c3)cc2)cc1]

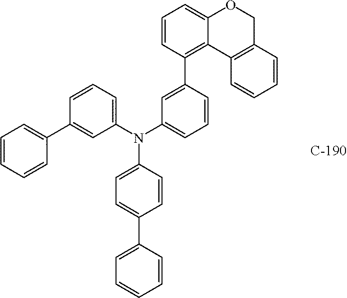 [SMILES: c1ccc(-c2ccc(N(c3cccc(-c4ccccc4)c3)c3cccc(-c4cccc5c4-c4ccccc4CO5)c3)cc2)cc1]

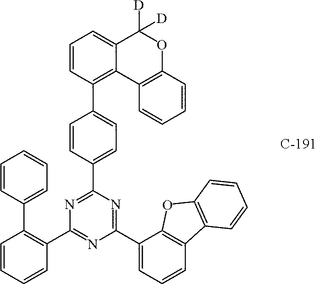 [SMILES: [2H]C1([2H])Oc2ccccc2-c2c(-c3ccc(-c4nc(-c5ccccc5-c5ccccc5)nc(-c5cccc6c5oc5ccccc56)n4)cc3)cccc21]

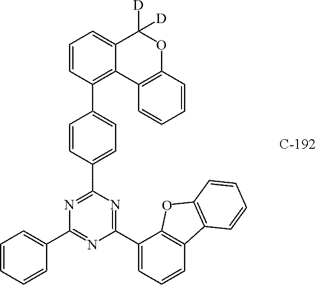 [SMILES: [2H]C1([2H])Oc2ccccc2-c2c(-c3ccc(-c4nc(-c5ccccc5)nc(-c5cccc6c5oc5ccccc56)n4)cc3)cccc21]

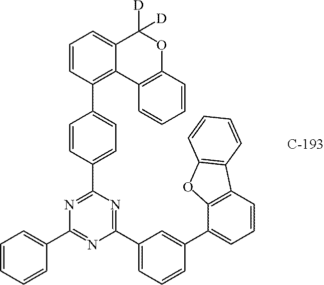 [SMILES: [2H]C1([2H])Oc2ccccc2-c2c(-c3ccc(-c4nc(-c5ccccc5)nc(-c5cccc(-c6cccc7c6oc6ccccc67)c5)n4)cc3)cccc21]

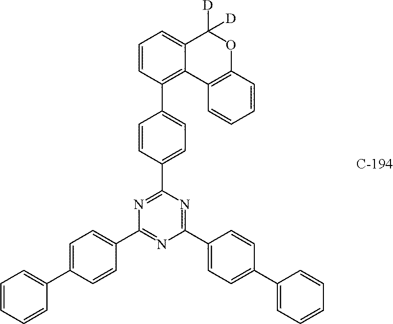 [SMILES: [2H]C1([2H])Oc2ccccc2-c2c(-c3ccc(-c4nc(-c5ccc(-c6ccccc6)cc5)nc(-c5ccc(-c6ccccc6)cc5)n4)cc3)cccc21]

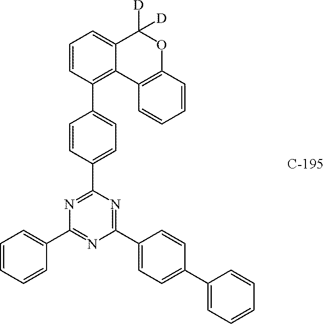 [SMILES: [2H]C1([2H])Oc2ccccc2-c2c(-c3ccc(-c4nc(-c5ccccc5)nc(-c5ccc(-c6ccccc6)cc5)n4)cc3)cccc21]

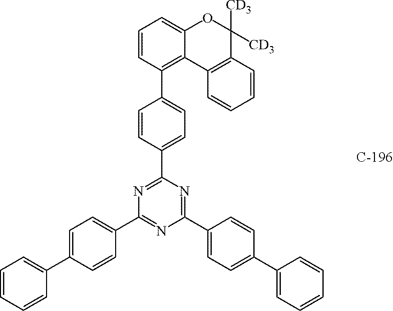 [SMILES: [2H]C([2H])([2H])C1(C([2H])([2H])[2H])Oc2cccc(-c3ccc(-c4nc(-c5ccc(-c6ccccc6)cc5)nc(-c5ccc(-c6ccccc6)cc5)n4)cc3)c2-c2ccccc21]

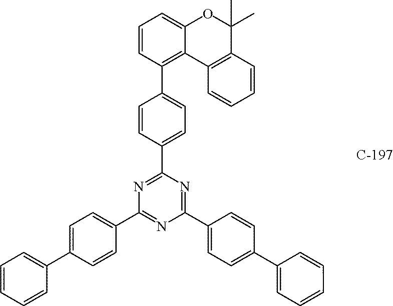 [SMILES: CC1(C)Oc2cccc(-c3ccc(-c4nc(-c5ccc(-c6ccccc6)cc5)nc(-c5ccc(-c6ccccc6)cc5)n4)cc3)c2-c2ccccc21]

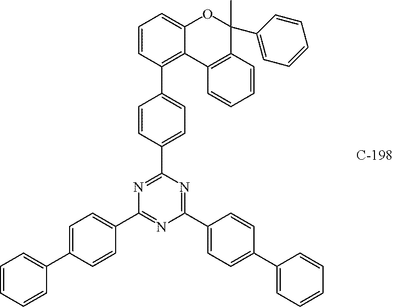 [SMILES: CC1(c2ccccc2)Oc2cccc(-c3ccc(-c4nc(-c5ccc(-c6ccccc6)cc5)nc(-c5ccc(-c6ccccc6)cc5)n4)cc3)c2-c2ccccc21]

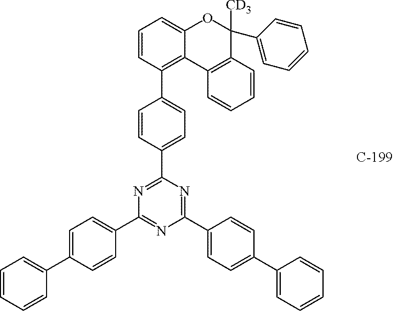 [SMILES: [2H]C([2H])([2H])C1(c2ccccc2)Oc2cccc(-c3ccc(-c4nc(-c5ccc(-c6ccccc6)cc5)nc(-c5ccc(-c6ccccc6)cc5)n4)cc3)c2-c2ccccc21]

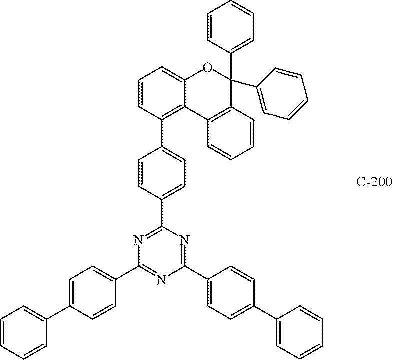 [SMILES: c1ccc(-c2ccc(-c3nc(-c4ccc(-c5ccccc5)cc4)nc(-c4ccc(-c5cccc6c5-c5ccccc5C(c5ccccc5)(c5ccccc5)O6)cc4)n3)cc2)cc1]

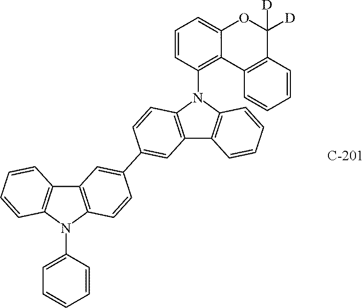 [SMILES: [2H]C1([2H])Oc2cccc(-n3c4ccccc4c4cc(-c5ccc6c(c5)c5ccccc5n6-c5ccccc5)ccc43)c2-c2ccccc21]

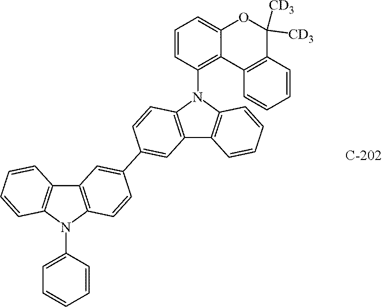 [SMILES: [2H]C([2H])([2H])C1(C([2H])([2H])[2H])Oc2cccc(-n3c4ccccc4c4cc(-c5ccc6c(c5)c5ccccc5n6-c5ccccc5)ccc43)c2-c2ccccc21]

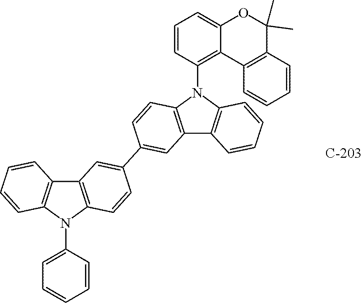 [SMILES: CC1(C)Oc2cccc(-n3c4ccccc4c4cc(-c5ccc6c(c5)c5ccccc5n6-c5ccccc5)ccc43)c2-c2ccccc21]

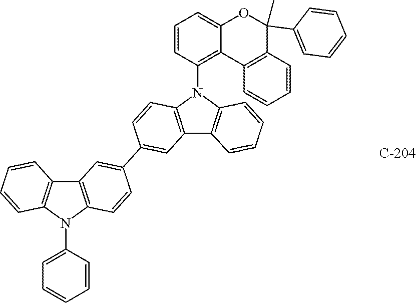 [SMILES: CC1(c2ccccc2)Oc2cccc(-n3c4ccccc4c4cc(-c5ccc6c(c5)c5ccccc5n6-c5ccccc5)ccc43)c2-c2ccccc21]

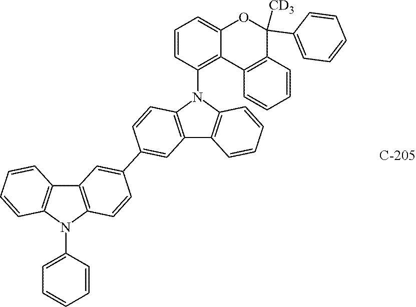 [SMILES: [2H]C([2H])([2H])C1(c2ccccc2)Oc2cccc(-n3c4ccccc4c4cc(-c5ccc6c(c5)c5ccccc5n6-c5ccccc5)ccc43)c2-c2ccccc21]

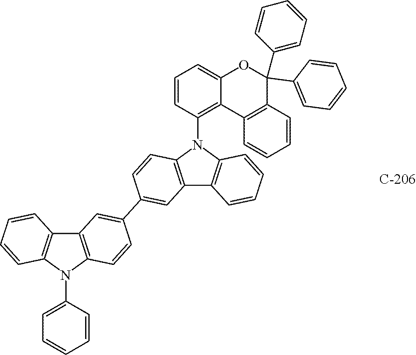 [SMILES: c1ccc(-n2c3ccccc3c3cc(-c4ccc5c(c4)c4ccccc4n5-c4cccc5c4-c4ccccc4C(c4ccccc4)(c4ccccc4)O5)ccc32)cc1]

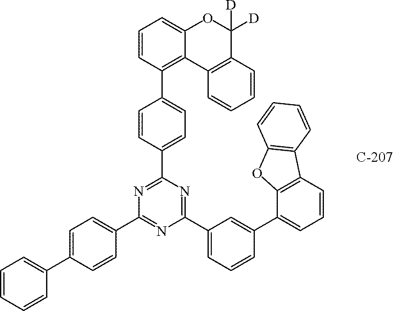 [SMILES: [2H]C1([2H])Oc2cccc(-c3ccc(-c4nc(-c5ccc(-c6ccccc6)cc5)nc(-c5cccc(-c6cccc7c6oc6ccccc67)c5)n4)cc3)c2-c2ccccc21]

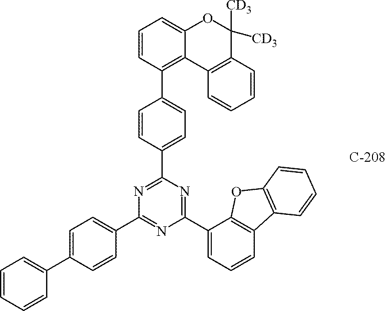 [SMILES: [2H]C([2H])([2H])C1(C([2H])([2H])[2H])Oc2cccc(-c3ccc(-c4nc(-c5ccc(-c6ccccc6)cc5)nc(-c5cccc6c5oc5ccccc56)n4)cc3)c2-c2ccccc21]

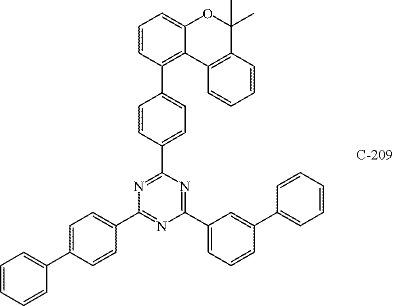 [SMILES: CC1(C)Oc2cccc(-c3ccc(-c4nc(-c5ccc(-c6ccccc6)cc5)nc(-c5cccc(-c6ccccc6)c5)n4)cc3)c2-c2ccccc21]

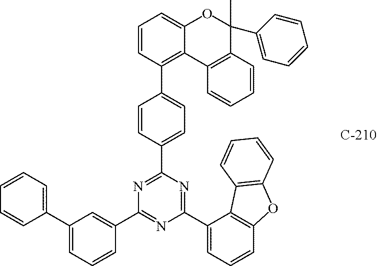 [SMILES: CC1(c2ccccc2)Oc2cccc(-c3ccc(-c4nc(-c5cccc(-c6ccccc6)c5)nc(-c5cccc6oc7ccccc7c56)n4)cc3)c2-c2ccccc21]

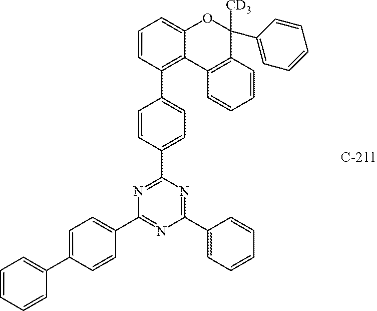 [SMILES: [2H]C([2H])([2H])C1(c2ccccc2)Oc2cccc(-c3ccc(-c4nc(-c5ccccc5)nc(-c5ccc(-c6ccccc6)cc5)n4)cc3)c2-c2ccccc21]

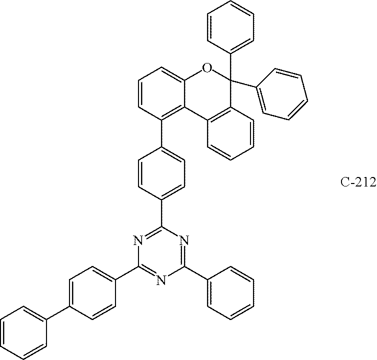 [SMILES: c1ccc(-c2ccc(-c3nc(-c4ccccc4)nc(-c4ccc(-c5cccc6c5-c5ccccc5C(c5ccccc5)(c5ccccc5)O6)cc4)n3)cc2)cc1]

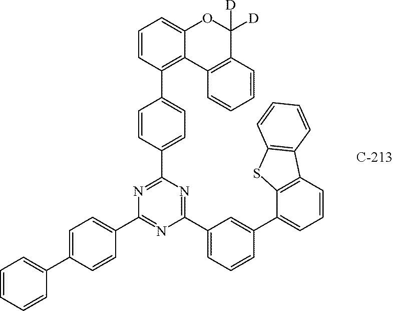 [SMILES: [2H]C1([2H])Oc2cccc(-c3ccc(-c4nc(-c5ccc(-c6ccccc6)cc5)nc(-c5cccc(-c6cccc7c6sc6ccccc67)c5)n4)cc3)c2-c2ccccc21]

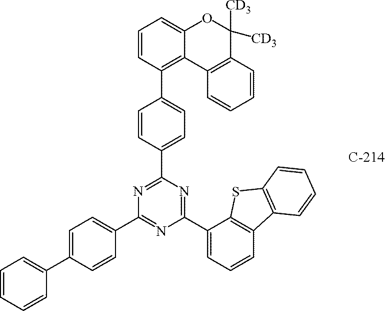 [SMILES: [2H]C([2H])([2H])C1(C([2H])([2H])[2H])Oc2cccc(-c3ccc(-c4nc(-c5ccc(-c6ccccc6)cc5)nc(-c5cccc6c5sc5ccccc56)n4)cc3)c2-c2ccccc21]

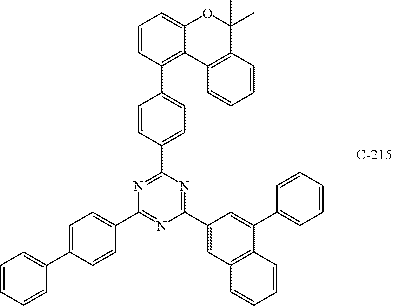 [SMILES: CC1(C)Oc2cccc(-c3ccc(-c4nc(-c5ccc(-c6ccccc6)cc5)nc(-c5cc(-c6ccccc6)c6ccccc6c5)n4)cc3)c2-c2ccccc21]

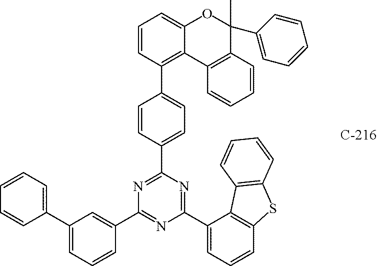 [SMILES: CC1(c2ccccc2)Oc2cccc(-c3ccc(-c4nc(-c5cccc(-c6ccccc6)c5)nc(-c5cccc6sc7ccccc7c56)n4)cc3)c2-c2ccccc21]

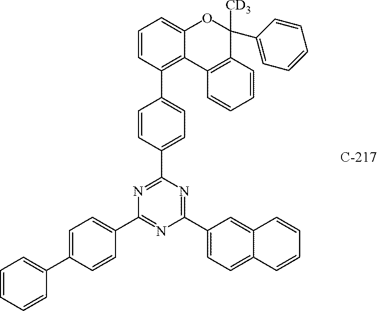 [SMILES: [2H]C([2H])([2H])C1(c2ccccc2)Oc2cccc(-c3ccc(-c4nc(-c5ccc(-c6ccccc6)cc5)nc(-c5ccc6ccccc6c5)n4)cc3)c2-c2ccccc21]

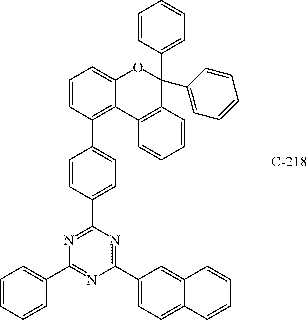 [SMILES: c1ccc(-c2nc(-c3ccc(-c4cccc5c4-c4ccccc4C(c4ccccc4)(c4ccccc4)O5)cc3)nc(-c3ccc4ccccc4c3)n2)cc1]

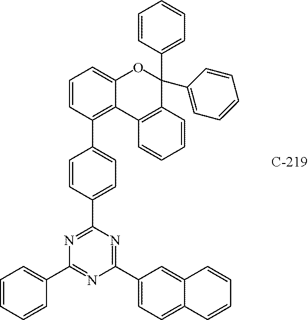 [SMILES: c1ccc(-c2nc(-c3ccc(-c4cccc5c4-c4ccccc4C(c4ccccc4)(c4ccccc4)O5)cc3)nc(-c3ccc4ccccc4c3)n2)cc1]

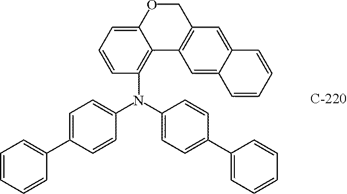 [SMILES: c1ccc(-c2ccc(N(c3ccc(-c4ccccc4)cc3)c3cccc4c3-c3cc5ccccc5cc3CO4)cc2)cc1]

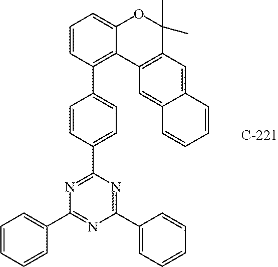 [SMILES: CC1(C)Oc2cccc(-c3ccc(-c4nc(-c5ccccc5)nc(-c5ccccc5)n4)cc3)c2-c2cc3ccccc3cc21]

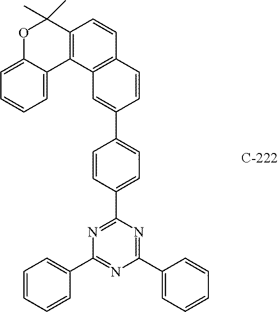 [SMILES: CC1(C)Oc2ccccc2-c2c1ccc1ccc(-c3ccc(-c4nc(-c5ccccc5)nc(-c5ccccc5)n4)cc3)cc21]

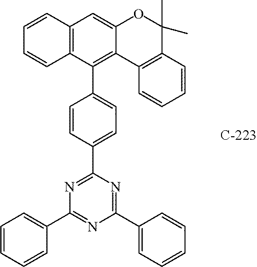 [SMILES: CC1(C)Oc2cc3ccccc3c(-c3ccc(-c4nc(-c5ccccc5)nc(-c5ccccc5)n4)cc3)c2-c2ccccc21]

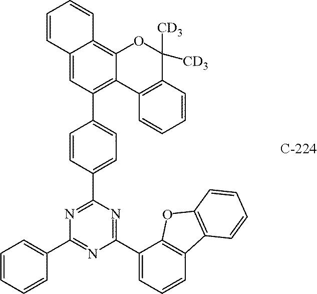 [SMILES: [2H]C([2H])([2H])C1(C([2H])([2H])[2H])Oc2c(c(-c3ccc(-c4nc(-c5ccccc5)nc(-c5cccc6c5oc5ccccc56)n4)cc3)cc3ccccc23)-c2ccccc21]

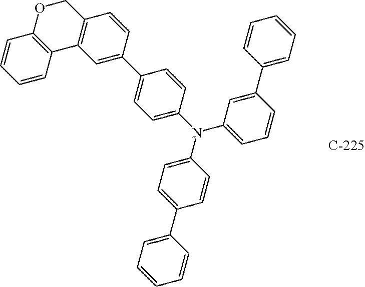 [SMILES: c1ccc(-c2ccc(N(c3ccc(-c4ccc5c(c4)-c4ccccc4OC5)cc3)c3cccc(-c4ccccc4)c3)cc2)cc1]

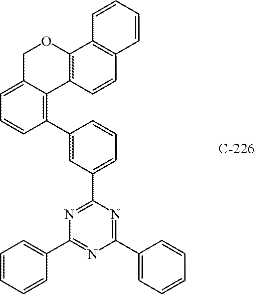 [SMILES: c1ccc(-c2nc(-c3ccccc3)nc(-c3cccc(-c4cccc5c4-c4ccc6ccccc6c4OC5)c3)n2)cc1]

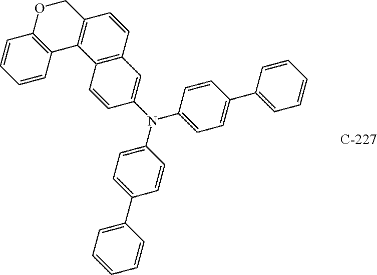 [SMILES: c1ccc(-c2ccc(N(c3ccc(-c4ccccc4)cc3)c3ccc4c5c(ccc4c3)COc3ccccc3-5)cc2)cc1]

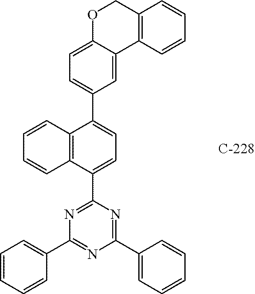 [SMILES: c1ccc(-c2nc(-c3ccccc3)nc(-c3ccc(-c4ccc5c(c4)-c4ccccc4CO5)c4ccccc34)n2)cc1]

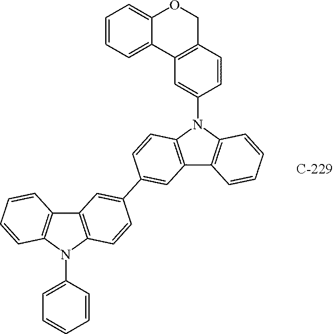 [SMILES: c1ccc(-n2c3ccccc3c3cc(-c4ccc5c(c4)c4ccccc4n5-c4ccc5c(c4)-c4ccccc4OC5)ccc32)cc1]

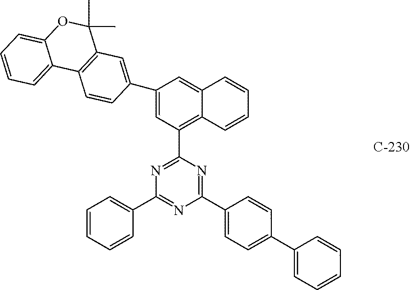 [SMILES: CC1(C)Oc2ccccc2-c2ccc(-c3cc(-c4nc(-c5ccccc5)nc(-c5ccc(-c6ccccc6)cc5)n4)c4ccccc4c3)cc21]

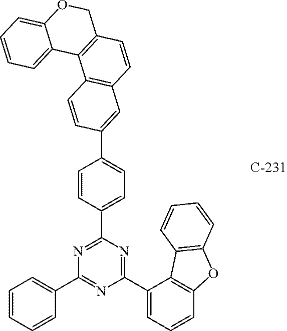 [SMILES: c1ccc(-c2nc(-c3ccc(-c4ccc5c6c(ccc5c4)COc4ccccc4-6)cc3)nc(-c3cccc4oc5ccccc5c34)n2)cc1]

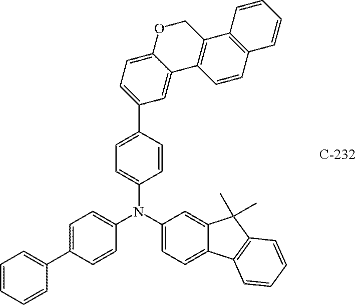 [SMILES: CC1(C)c2ccccc2-c2ccc(N(c3ccc(-c4ccccc4)cc3)c3ccc(-c4ccc5c(c4)-c4ccc6ccccc6c4CO5)cc3)cc21]

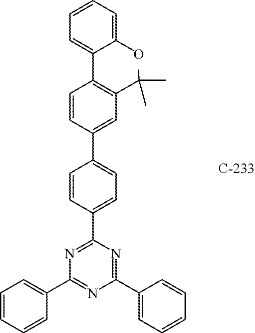 [SMILES: CC(C)(C)c1cc(-c2ccc(-c3nc(-c4ccccc4)nc(-c4ccccc4)n3)cc2)ccc1-c1ccccc1O]

The compound represented by formula 2 may be at least one selected from the group consisting of the following compounds, but is not limited thereto.

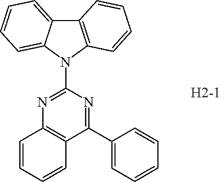 [SMILES: c1ccc(-c2nc(-n3c4ccccc4c4ccccc43)nc3ccccc23)cc1]

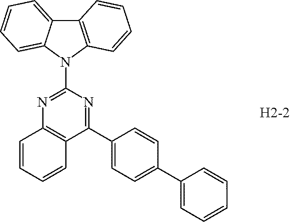 [SMILES: c1ccc(-c2ccc(-c3nc(-n4c5ccccc5c5ccccc54)nc4ccccc34)cc2)cc1]

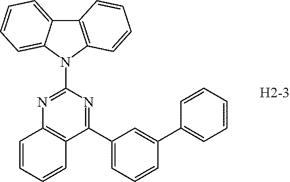 [SMILES: c1ccc(-c2cccc(-c3nc(-n4c5ccccc5c5ccccc54)nc4ccccc34)c2)cc1]

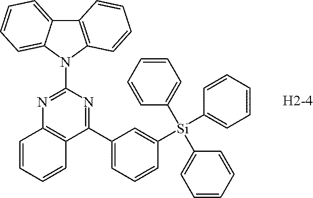 [SMILES: c1ccc([Si](c2ccccc2)(c2ccccc2)c2cccc(-c3nc(-n4c5ccccc5c5ccccc54)nc4ccccc34)c2)cc1]

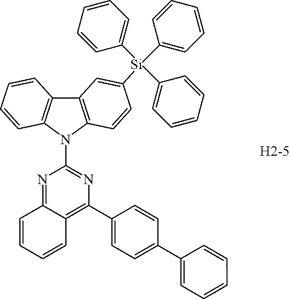 [SMILES: c1ccc(-c2ccc(-c3nc(-n4c5ccccc5c5cc([Si](c6ccccc6)(c6ccccc6)c6ccccc6)ccc54)nc4ccccc34)cc2)cc1]

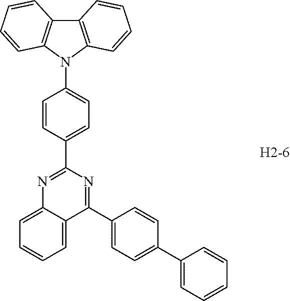 [SMILES: c1ccc(-c2ccc(-c3nc(-c4ccc(-n5c6ccccc6c6ccccc65)cc4)nc4ccccc34)cc2)cc1]

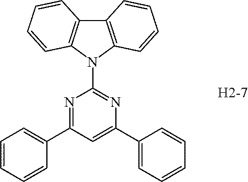 [SMILES: c1ccc(-c2cc(-c3ccccc3)nc(-n3c4ccccc4c4ccccc43)n2)cc1]

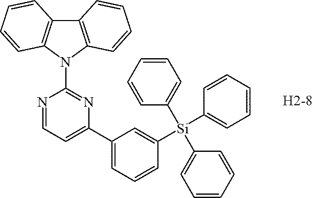 [SMILES: c1ccc([Si](c2ccccc2)(c2ccccc2)c2cccc(-c3ccnc(-n4c5ccccc5c5ccccc54)n3)c2)cc1]

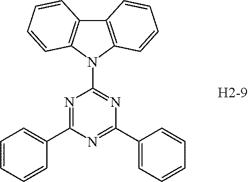 [SMILES: c1ccc(-c2nc(-c3ccccc3)nc(-n3c4ccccc4c4ccccc43)n2)cc1]

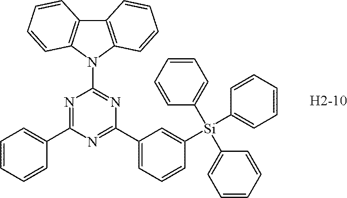 [SMILES: c1ccc(-c2nc(-c3cccc([Si](c4ccccc4)(c4ccccc4)c4ccccc4)c3)nc(-n3c4ccccc4c4ccccc43)n2)cc1]

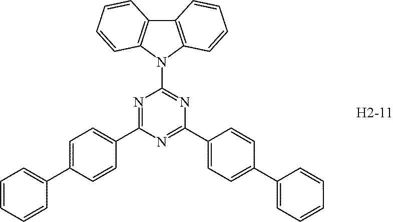 [SMILES: c1ccc(-c2ccc(-c3nc(-c4ccc(-c5ccccc5)cc4)nc(-n4c5ccccc5c5ccccc54)n3)cc2)cc1]

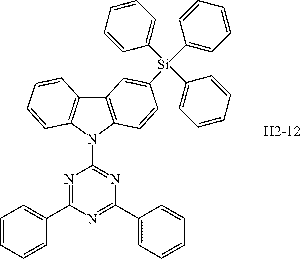 [SMILES: c1ccc(-c2nc(-c3ccccc3)nc(-n3c4ccccc4c4cc([Si](c5ccccc5)(c5ccccc5)c5ccccc5)ccc43)n2)cc1]

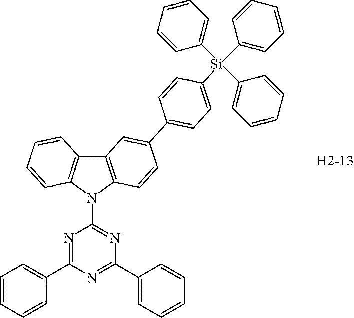 [SMILES: c1ccc(-c2nc(-c3ccccc3)nc(-n3c4ccccc4c4cc(-c5ccc([Si](c6ccccc6)(c6ccccc6)c6ccccc6)cc5)ccc43)n2)cc1]

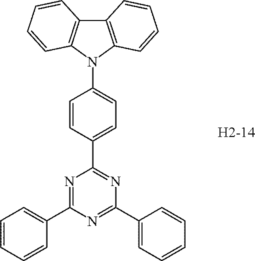 [SMILES: c1ccc(-c2nc(-c3ccccc3)nc(-c3ccc(-n4c5ccccc5c5ccccc54)cc3)n2)cc1]

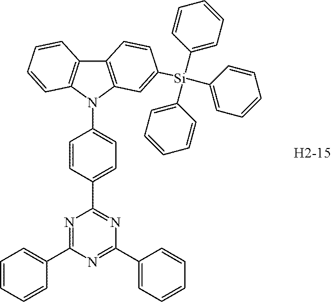 [SMILES: c1ccc(-c2nc(-c3ccccc3)nc(-c3ccc(-n4c5ccccc5c5ccc([Si](c6ccccc6)(c6ccccc6)c6ccccc6)cc54)cc3)n2)cc1]

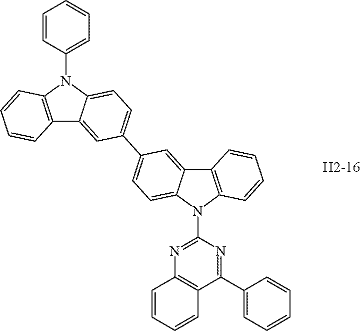 [SMILES: c1ccc(-c2nc(-n3c4ccccc4c4cc(-c5ccc6c(c5)c5ccccc5n6-c5ccccc5)ccc43)nc3ccccc23)cc1]

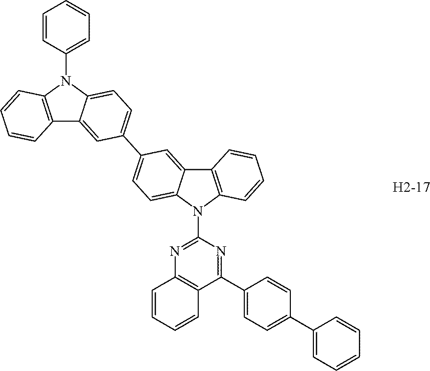 [SMILES: c1ccc(-c2ccc(-c3nc(-n4c5ccccc5c5cc(-c6ccc7c(c6)c6ccccc6n7-c6ccccc6)ccc54)nc4ccccc34)cc2)cc1]

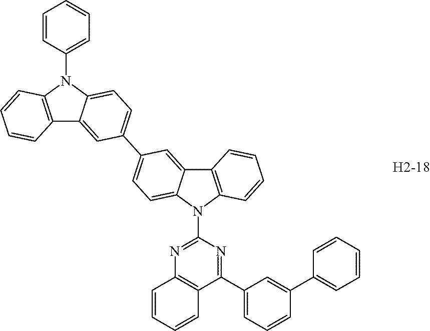 [SMILES: c1ccc(-c2cccc(-c3nc(-n4c5ccccc5c5cc(-c6ccc7c(c6)c6ccccc6n7-c6ccccc6)ccc54)nc4ccccc34)c2)cc1]

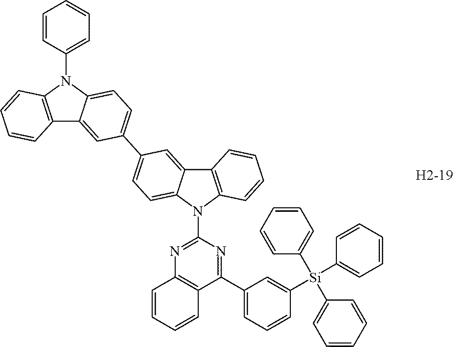 [SMILES: c1ccc(-n2c3ccccc3c3cc(-c4ccc5c(c4)c4ccccc4n5-c4nc(-c5cccc([Si](c6ccccc6)(c6ccccc6)c6ccccc6)c5)c5ccccc5n4)ccc32)cc1]

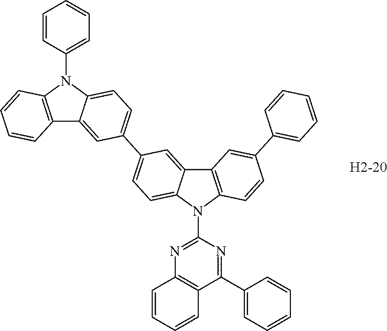 [SMILES: c1ccc(-c2ccc3c(c2)c2cc(-c4ccc5c(c4)c4ccccc4n5-c4ccccc4)ccc2n3-c2nc(-c3ccccc3)c3ccccc3n2)cc1]

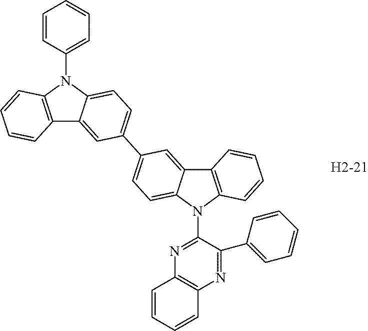 [SMILES: c1ccc(-c2nc3ccccc3nc2-n2c3ccccc3c3cc(-c4ccc5c(c4)c4ccccc4n5-c4ccccc4)ccc32)cc1]

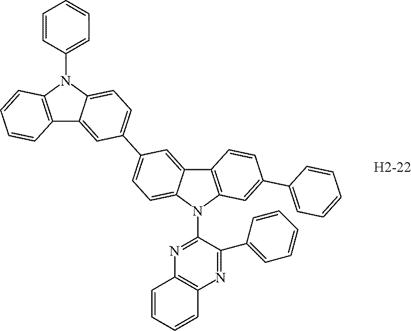 [SMILES: c1ccc(-c2ccc3c4cc(-c5ccc6c(c5)c5ccccc5n6-c5ccccc5)ccc4n(-c4nc5ccccc5nc4-c4ccccc4)c3c2)cc1]

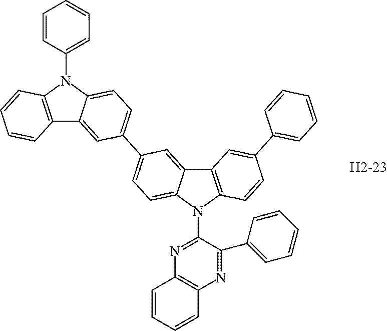 [SMILES: c1ccc(-c2ccc3c(c2)c2cc(-c4ccc5c(c4)c4ccccc4n5-c4ccccc4)ccc2n3-c2nc3ccccc3nc2-c2ccccc2)cc1]

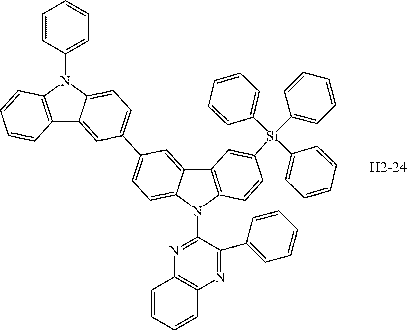 [SMILES: c1ccc(-c2nc3ccccc3nc2-n2c3ccc(-c4ccc5c(c4)c4ccccc4n5-c4ccccc4)cc3c3cc([Si](c4ccccc4)(c4ccccc4)c4ccccc4)ccc32)cc1]

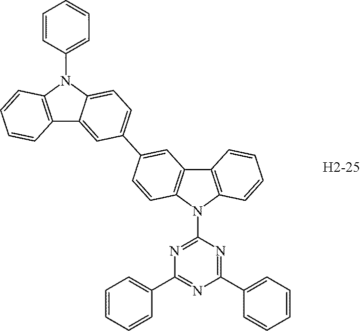 [SMILES: c1ccc(-c2nc(-c3ccccc3)nc(-n3c4ccccc4c4cc(-c5ccc6c(c5)c5ccccc5n6-c5ccccc5)ccc43)n2)cc1]

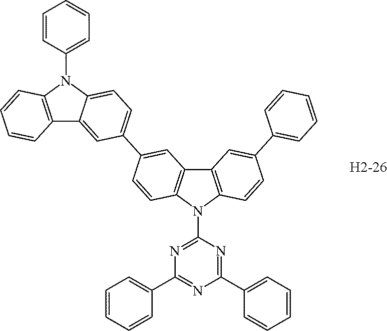 [SMILES: c1ccc(-c2ccc3c(c2)c2cc(-c4ccc5c(c4)c4ccccc4n5-c4ccccc4)ccc2n3-c2nc(-c3ccccc3)nc(-c3ccccc3)n2)cc1]

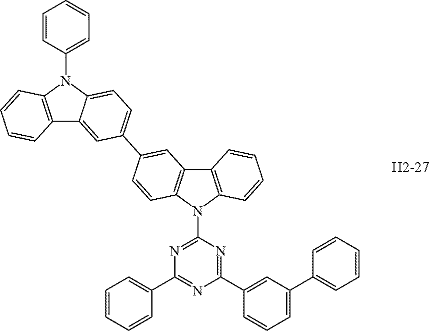 [SMILES: c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1]

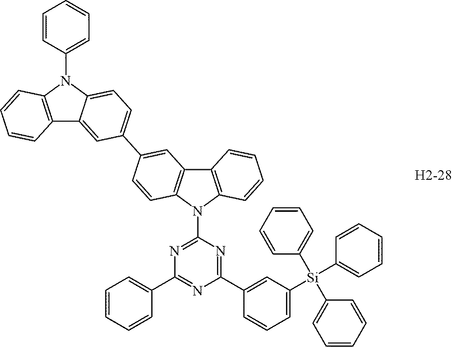 [SMILES: c1ccc(-c2nc(-c3cccc([Si](c4ccccc4)(c4ccccc4)c4ccccc4)c3)nc(-n3c4ccccc4c4cc(-c5ccc6c(c5)c5ccccc5n6-c5ccccc5)ccc43)n2)cc1]

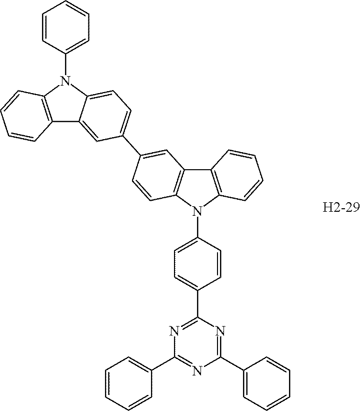 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

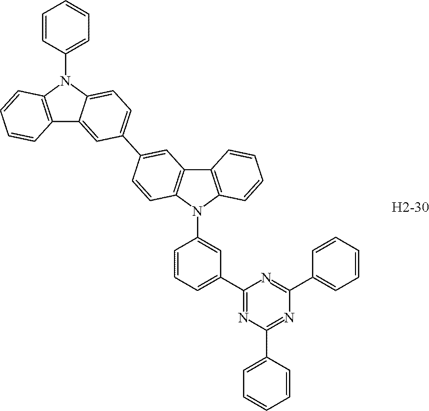 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

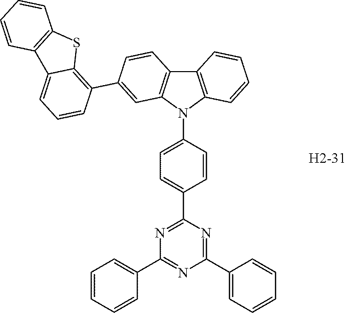 [SMILES: c1ccc(-c2nc(-c3ccccc3)nc(-c3ccc(-n4c5ccccc5c5ccc(-c6cccc7c6sc6ccccc67)cc54)cc3)n2)cc1]

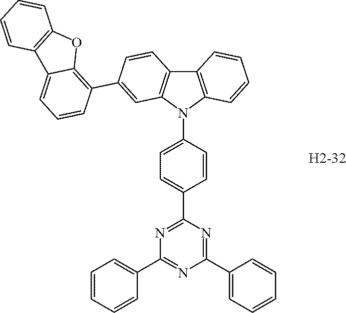 [SMILES: c1ccc(-c2nc(-c3ccccc3)nc(-c3ccc(-n4c5ccccc5c5ccc(-c6cccc7c6oc6ccccc67)cc54)cc3)n2)cc1]

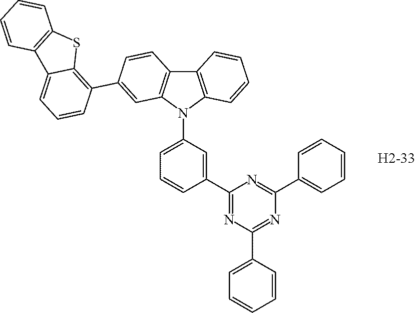 [SMILES: c1ccc(-c2nc(-c3ccccc3)nc(-c3cccc(-n4c5ccccc5c5ccc(-c6cccc7c6sc6ccccc67)cc54)c3)n2)cc1]

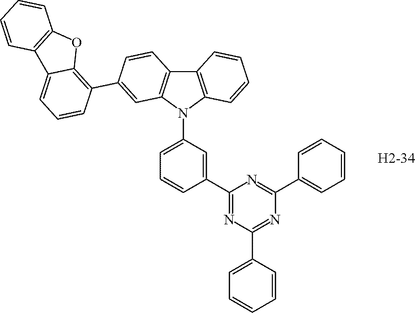 [SMILES: c1ccc(-c2nc(-c3ccccc3)nc(-c3cccc(-n4c5ccccc5c5ccc(-c6cccc7c6oc6ccccc67)cc54)c3)n2)cc1]

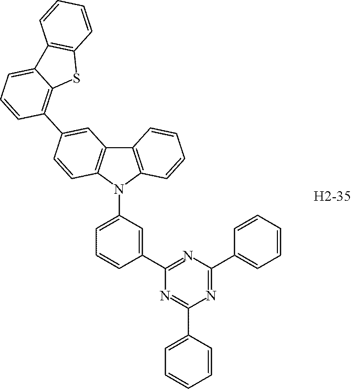 [SMILES: c1ccc(-c2nc(-c3ccccc3)nc(-c3cccc(-n4c5ccccc5c5cc(-c6cccc7c6sc6ccccc67)ccc54)c3)n2)cc1]

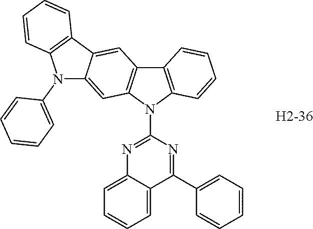 [SMILES: c1ccc(-c2nc(-n3c4ccccc4c4cc5c6ccccc6n(-c6ccccc6)c5cc43)nc3ccccc23)cc1]

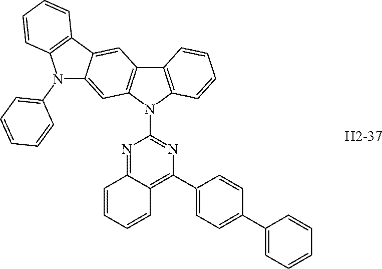 [SMILES: c1ccc(-c2ccc(-c3nc(-n4c5ccccc5c5cc6c7ccccc7n(-c7ccccc7)c6cc54)nc4ccccc34)cc2)cc1]

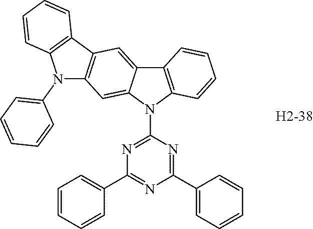 [SMILES: c1ccc(-c2nc(-c3ccccc3)nc(-n3c4ccccc4c4cc5c6ccccc6n(-c6ccccc6)c5cc43)n2)cc1]

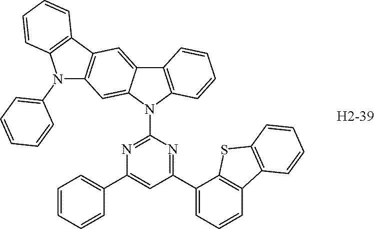 [SMILES: c1ccc(-c2cc(-c3cccc4c3sc3ccccc34)nc(-n3c4ccccc4c4cc5c6ccccc6n(-c6ccccc6)c5cc43)n2)cc1]

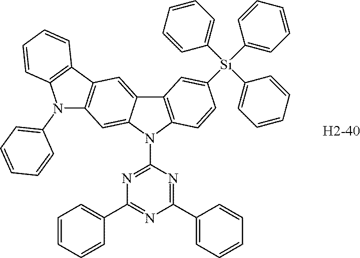 [SMILES: c1ccc(-c2nc(-c3ccccc3)nc(-n3c4ccc([Si](c5ccccc5)(c5ccccc5)c5ccccc5)cc4c4cc5c6ccccc6n(-c6ccccc6)c5cc43)n2)cc1]

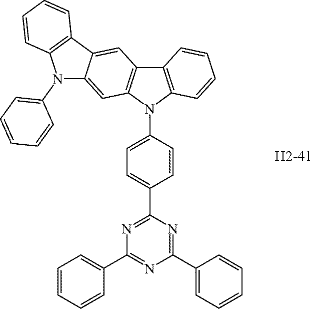 [SMILES: c1ccc(-c2nc(-c3ccccc3)nc(-c3ccc(-n4c5ccccc5c5cc6c7ccccc7n(-c7ccccc7)c6cc54)cc3)n2)cc1]

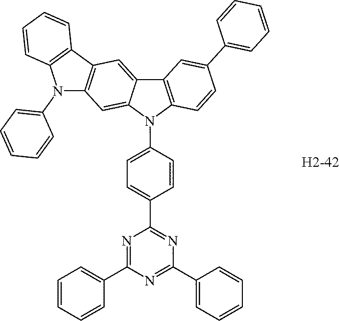 [SMILES: c1ccc(-c2ccc3c(c2)c2cc4c5ccccc5n(-c5ccccc5)c4cc2n3-c2ccc(-c3nc(-c4ccccc4)nc(-c4ccccc4)n3)cc2)cc1]

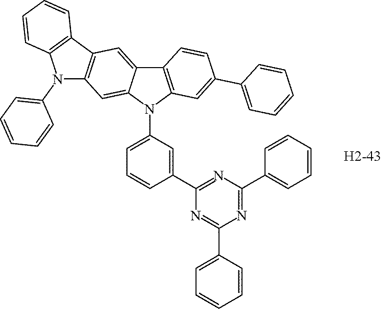 [SMILES: c1ccc(-c2ccc3c4cc5c6ccccc6n(-c6ccccc6)c5cc4n(-c4cccc(-c5nc(-c6ccccc6)nc(-c6ccccc6)n5)c4)c3c2)cc1]

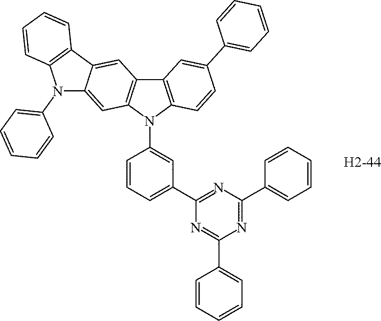 [SMILES: c1ccc(-c2ccc3c(c2)c2cc4c5ccccc5n(-c5ccccc5)c4cc2n3-c2cccc(-c3nc(-c4ccccc4)nc(-c4ccccc4)n3)c2)cc1]

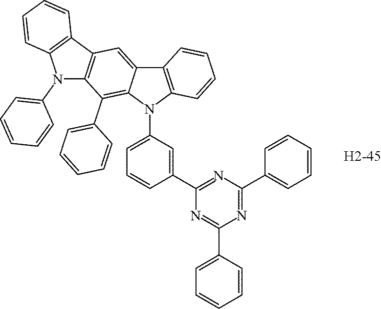 [SMILES: c1ccc(-c2nc(-c3ccccc3)nc(-c3cccc(-n4c5ccccc5c5cc6c7ccccc7n(-c7ccccc7)c6c(-c6ccccc6)c54)c3)n2)cc1]

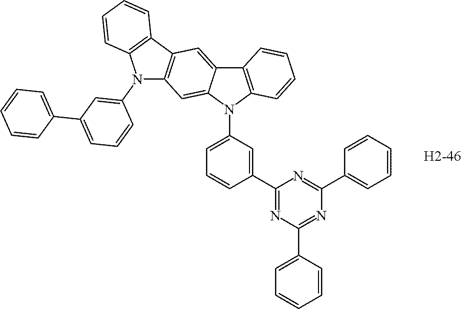 [SMILES: c1ccc(-c2cccc(-n3c4ccccc4c4cc5c6ccccc6n(-c6cccc(-c7nc(-c8ccccc8)nc(-c8ccccc8)n7)c6)c5cc43)c2)cc1]

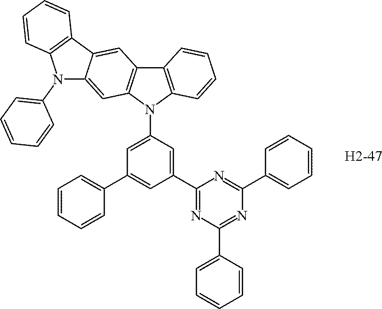 [SMILES: c1ccc(-c2cc(-c3nc(-c4ccccc4)nc(-c4ccccc4)n3)cc(-n3c4ccccc4c4cc5c6ccccc6n(-c6ccccc6)c5cc43)c2)cc1]

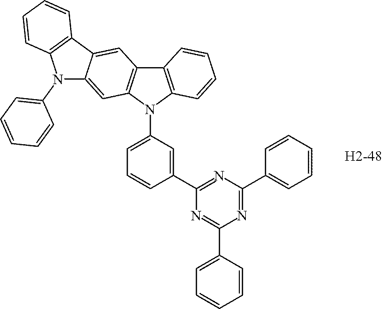 [SMILES: c1ccc(-c2nc(-c3ccccc3)nc(-c3cccc(-n4c5ccccc5c5cc6c7ccccc7n(-c7ccccc7)c6cc54)c3)n2)cc1]

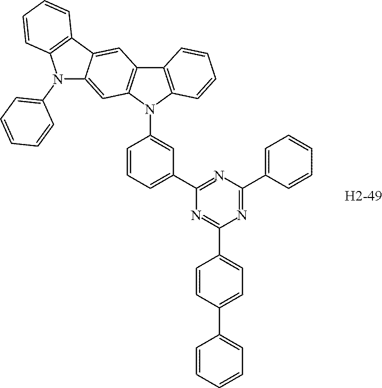 [SMILES: c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1]

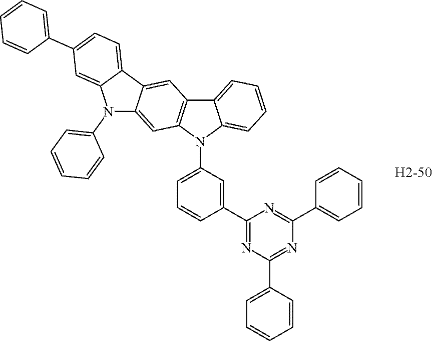 [SMILES: c1ccc(-c2ccc3c4cc5c6ccccc6n(-c6cccc(-c7nc(-c8ccccc8)nc(-c8ccccc8)n7)c6)c5cc4n(-c4ccccc4)c3c2)cc1]

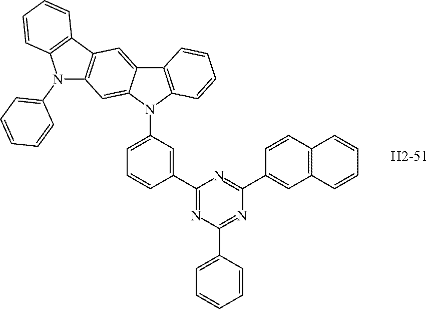 [SMILES: c1ccc(-c2nc(-c3cccc(-n4c5ccccc5c5cc6c7ccccc7n(-c7ccccc7)c6cc54)c3)nc(-c3ccc4ccccc4c3)n2)cc1]

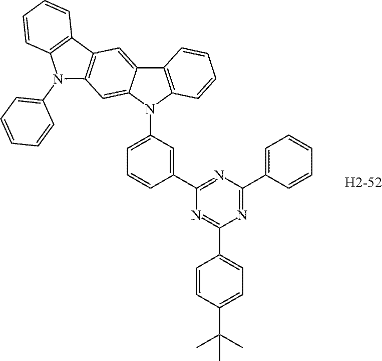 [SMILES: CC(C)(C)c1ccc(-c2nc(-c3ccccc3)nc(-c3cccc(-n4c5ccccc5c5cc6c7ccccc7n(-c7ccccc7)c6cc54)c3)n2)cc1]

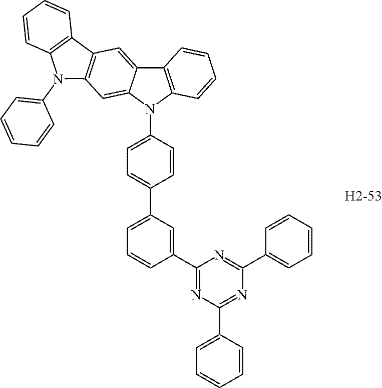 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1]

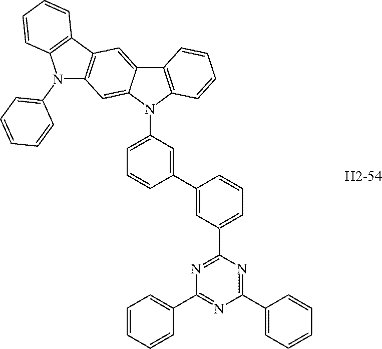 [SMILES: c1ccc(-c2nc(-c3ccccc3)nc(-c3cccc(-c4cccc(-n5c6ccccc6c6cc7c8ccccc8n(-c8ccccc8)c7cc65)c4)c3)n2)cc1]

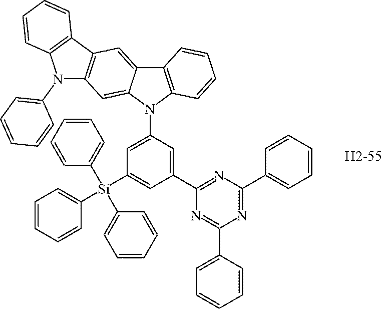 [SMILES: c1ccc(-c2nc(-c3ccccc3)nc(-c3cc(-n4c5ccccc5c5cc6c7ccccc7n(-c7ccccc7)c6cc54)cc([Si](c4ccccc4)(c4ccccc4)c4ccccc4)c3)n2)cc1]

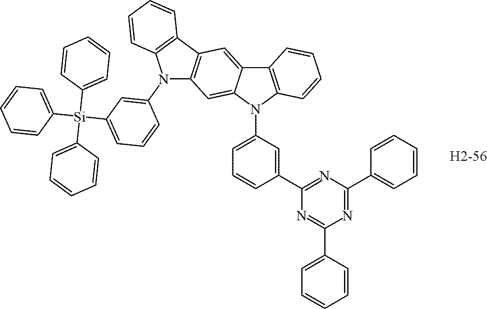 [SMILES: c1ccc(-c2nc(-c3ccccc3)nc(-c3cccc(-n4c5ccccc5c5cc6c7ccccc7n(-c7cccc([Si](c8ccccc8)(c8ccccc8)c8ccccc8)c7)c6cc54)c3)n2)cc1]

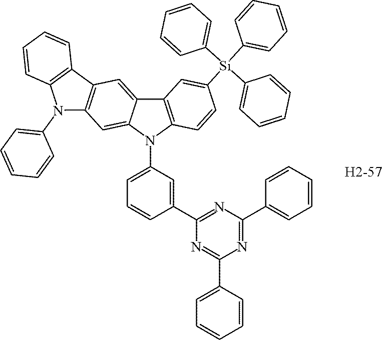 [SMILES: c1ccc(-c2nc(-c3ccccc3)nc(-c3cccc(-n4c5ccc([Si](c6ccccc6)(c6ccccc6)c6ccccc6)cc5c5cc6c7ccccc7n(-c7ccccc7)c6cc54)c3)n2)cc1]

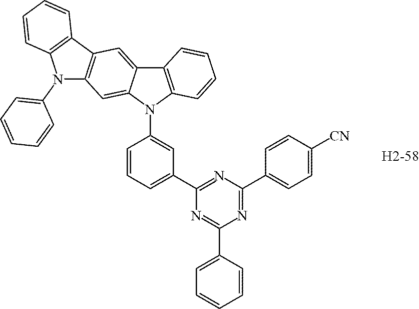 [SMILES: N#Cc1ccc(-c2nc(-c3ccccc3)nc(-c3cccc(-n4c5ccccc5c5cc6c7ccccc7n(-c7ccccc7)c6cc54)c3)n2)cc1]

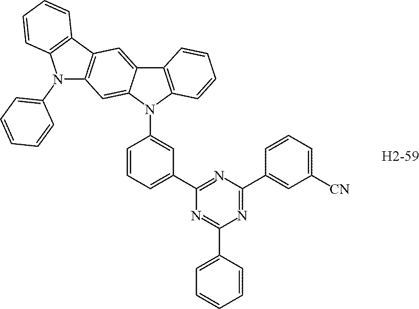 [SMILES: N#Cc1cccc(-c2nc(-c3ccccc3)nc(-c3cccc(-n4c5ccccc5c5cc6c7ccccc7n(-c7ccccc7)c6cc54)c3)n2)c1]

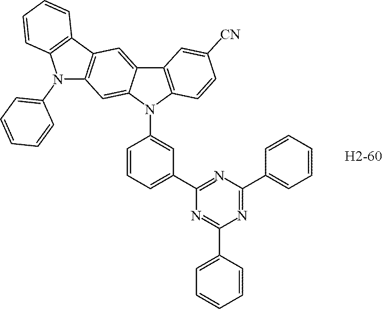 [SMILES: N#Cc1ccc2c(c1)c1cc3c4ccccc4n(-c4ccccc4)c3cc1n2-c1cccc(-c2nc(-c3ccccc3)nc(-c3ccccc3)n2)c1]

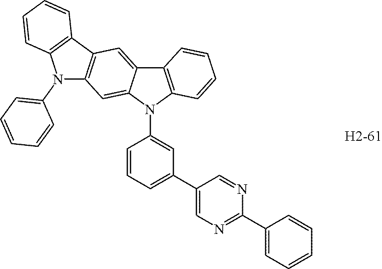 [SMILES: c1ccc(-c2ncc(-c3cccc(-n4c5ccccc5c5cc6c7ccccc7n(-c7ccccc7)c6cc54)c3)cn2)cc1]

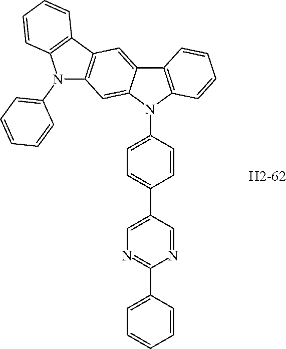 [SMILES: c1ccc(-c2ncc(-c3ccc(-n4c5ccccc5c5cc6c7ccccc7n(-c7ccccc7)c6cc54)cc3)cn2)cc1]

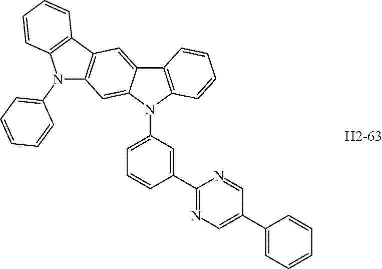 [SMILES: c1ccc(-c2cnc(-c3cccc(-n4c5ccccc5c5cc6c7ccccc7n(-c7ccccc7)c6cc54)c3)nc2)cc1]

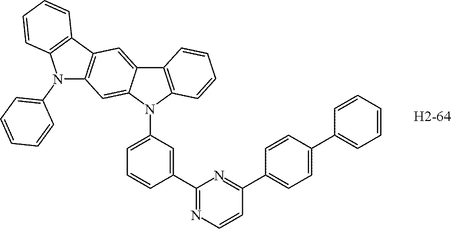 [SMILES: c1ccc(-c2ccc(-c3ccnc(-c4cccc(-n5c6ccccc6c6cc7c8ccccc8n(-c8ccccc8)c7cc65)c4)n3)cc2)cc1]

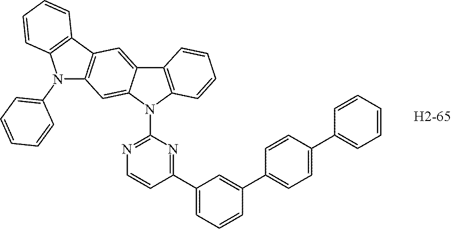 [SMILES: c1ccc(-c2ccc(-c3cccc(-c4ccnc(-n5c6ccccc6c6cc7c8ccccc8n(-c8ccccc8)c7cc65)n4)c3)cc2)cc1]

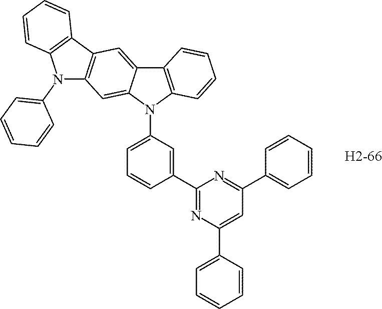 [SMILES: c1ccc(-c2cc(-c3ccccc3)nc(-c3cccc(-n4c5ccccc5c5cc6c7ccccc7n(-c7ccccc7)c6cc54)c3)n2)cc1]

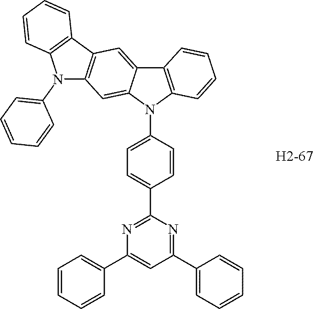 [SMILES: c1ccc(-c2cc(-c3ccccc3)nc(-c3ccc(-n4c5ccccc5c5cc6c7ccccc7n(-c7ccccc7)c6cc54)cc3)n2)cc1]

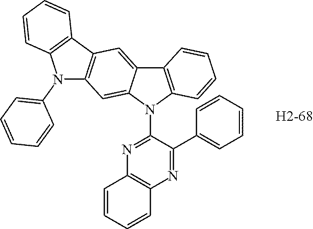 [SMILES: c1ccc(-c2nc3ccccc3nc2-n2c3ccccc3c3cc4c5ccccc5n(-c5ccccc5)c4cc32)cc1]

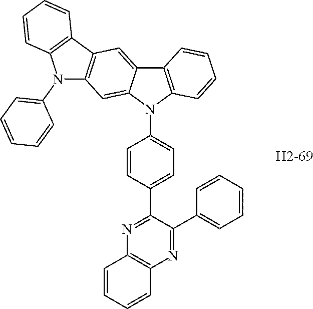 [SMILES: c1ccc(-c2nc3ccccc3nc2-c2ccc(-n3c4ccccc4c4cc5c6ccccc6n(-c6ccccc6)c5cc43)cc2)cc1]

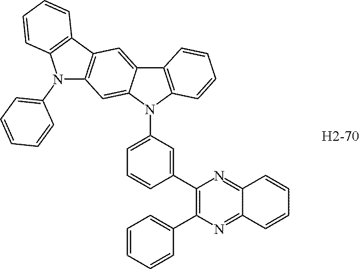 [SMILES: c1ccc(-c2nc3ccccc3nc2-c2cccc(-n3c4ccccc4c4cc5c6ccccc6n(-c6ccccc6)c5cc43)c2)cc1]

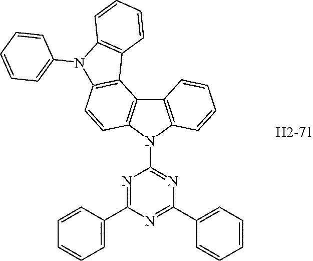 [SMILES: c1ccc(-c2nc(-c3ccccc3)nc(-n3c4ccccc4c4c5c6ccccc6n(-c6ccccc6)c5ccc43)n2)cc1]

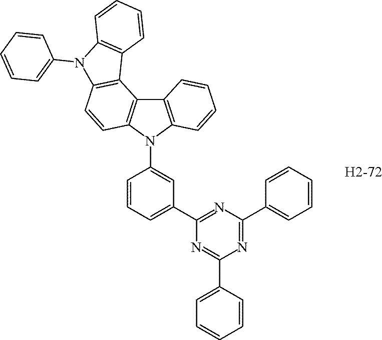 [SMILES: c1ccc(-c2nc(-c3ccccc3)nc(-c3cccc(-n4c5ccccc5c5c6c7ccccc7n(-c7ccccc7)c6ccc54)c3)n2)cc1]

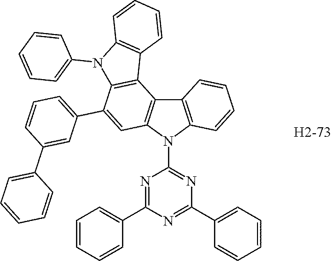 [SMILES: c1ccc(-c2cccc(-c3cc4c(c5ccccc5n4-c4nc(-c5ccccc5)nc(-c5ccccc5)n4)c4c5ccccc5n(-c5ccccc5)c34)c2)cc1]

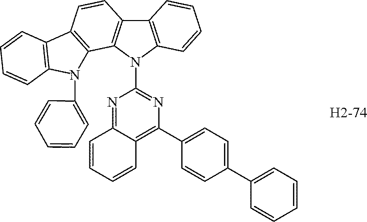 [SMILES: c1ccc(-c2ccc(-c3nc(-n4c5ccccc5c5ccc6c7ccccc7n(-c7ccccc7)c6c54)nc4ccccc34)cc2)cc1]

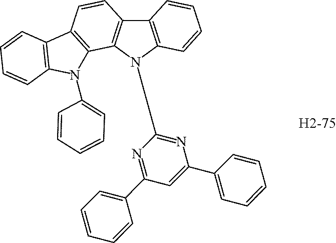 [SMILES: c1ccc(-c2cc(-c3ccccc3)nc(-n3c4ccccc4c4ccc5c6ccccc6n(-c6ccccc6)c5c43)n2)cc1]

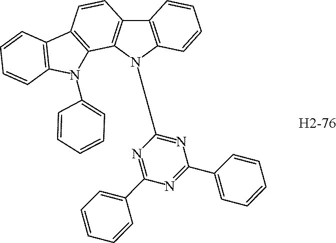 [SMILES: c1ccc(-c2nc(-c3ccccc3)nc(-n3c4ccccc4c4ccc5c6ccccc6n(-c6ccccc6)c5c43)n2)cc1]

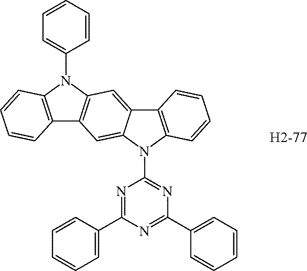 [SMILES: c1ccc(-c2nc(-c3ccccc3)nc(-n3c4ccccc4c4cc5c(cc43)c3ccccc3n5-c3ccccc3)n2)cc1]

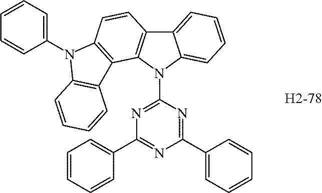 [SMILES: c1ccc(-c2nc(-c3ccccc3)nc(-n3c4ccccc4c4ccc5c(c6ccccc6n5-c5ccccc5)c43)n2)cc1]

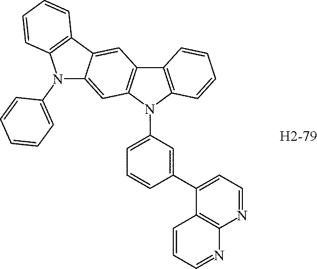 [SMILES: c1ccc(-n2c3ccccc3c3cc4c5ccccc5n(-c5cccc(-c6ccnc7ncccc67)c5)c4cc32)cc1]

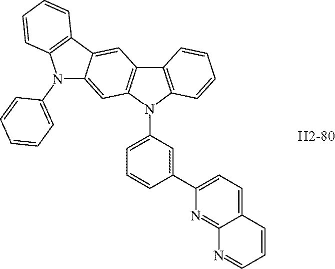 [SMILES: c1ccc(-n2c3ccccc3c3cc4c5ccccc5n(-c5cccc(-c6ccc7cccnc7n6)c5)c4cc32)cc1]

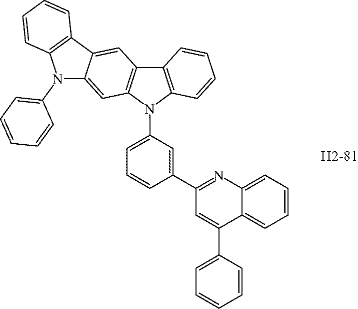 [SMILES: c1ccc(-c2cc(-c3cccc(-n4c5ccccc5c5cc6c7ccccc7n(-c7ccccc7)c6cc54)c3)nc3ccccc23)cc1]

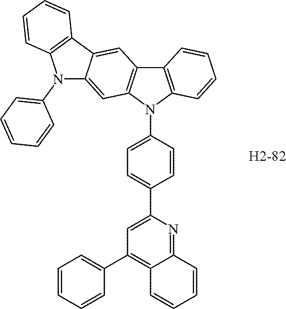 [SMILES: c1ccc(-c2cc(-c3ccc(-n4c5ccccc5c5cc6c7ccccc7n(-c7ccccc7)c6cc54)cc3)nc3ccccc23)cc1]

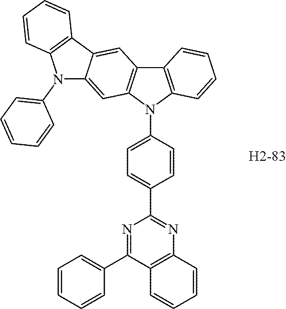 [SMILES: c1ccc(-c2nc(-c3ccc(-n4c5ccccc5c5cc6c7ccccc7n(-c7ccccc7)c6cc54)cc3)nc3ccccc23)cc1]

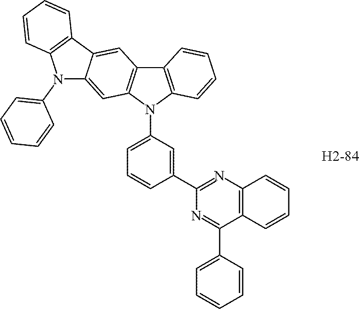 [SMILES: c1ccc(-c2nc(-c3cccc(-n4c5ccccc5c5cc6c7ccccc7n(-c7ccccc7)c6cc54)c3)nc3ccccc23)cc1]

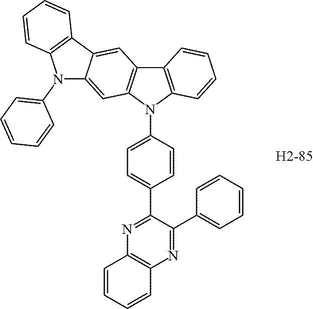 [SMILES: c1ccc(-c2nc3ccccc3nc2-c2ccc(-n3c4ccccc4c4cc5c6ccccc6n(-c6ccccc6)c5cc43)cc2)cc1]

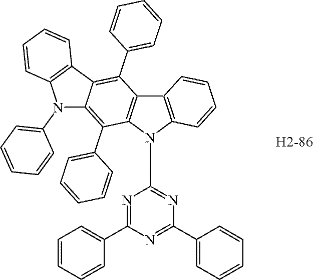 [SMILES: c1ccc(-c2nc(-c3ccccc3)nc(-n3c4ccccc4c4c(-c5ccccc5)c5c6ccccc6n(-c6ccccc6)c5c(-c5ccccc5)c43)n2)cc1]

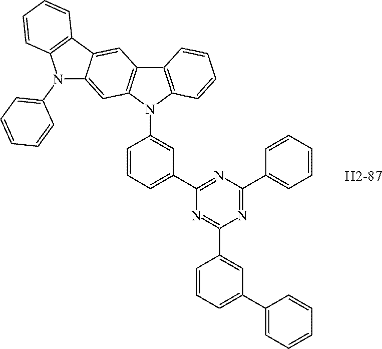 [SMILES: c1ccc(-c2cccc(-c3nc(-c4ccccc4)nc(-c4cccc(-n5c6ccccc6c6cc7c8ccccc8n(-c8ccccc8)c7cc65)c4)n3)c2)cc1]

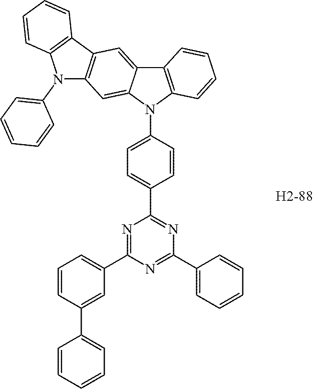 [SMILES: c1ccc(-c2cccc(-c3nc(-c4ccccc4)nc(-c4ccc(-n5c6ccccc6c6cc7c8ccccc8n(-c8ccccc8)c7cc65)cc4)n3)c2)cc1]

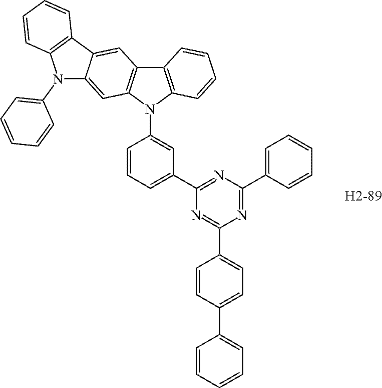 [SMILES: c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1]

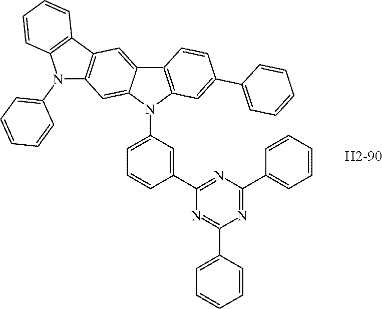 [SMILES: c1ccc(-c2ccc3c4cc5c6ccccc6n(-c6ccccc6)c5cc4n(-c4cccc(-c5nc(-c6ccccc6)nc(-c6ccccc6)n5)c4)c3c2)cc1]

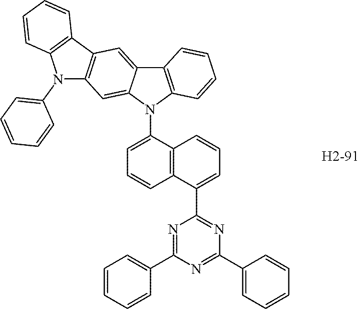 [SMILES: c1ccc(-c2nc(-c3ccccc3)nc(-c3cccc4c(-n5c6ccccc6c6cc7c8ccccc8n(-c8ccccc8)c7cc65)cccc34)n2)cc1]

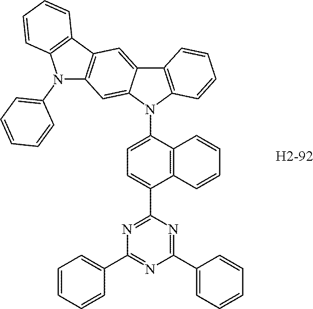 [SMILES: c1ccc(-c2nc(-c3ccccc3)nc(-c3ccc(-n4c5ccccc5c5cc6c7ccccc7n(-c7ccccc7)c6cc54)c4ccccc34)n2)cc1]

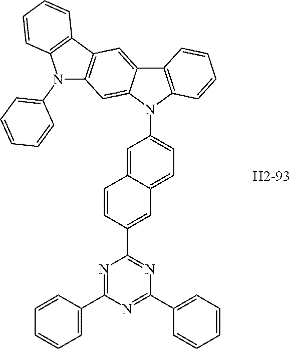 [SMILES: c1ccc(-c2nc(-c3ccccc3)nc(-c3ccc4cc(-n5c6ccccc6c6cc7c8ccccc8n(-c8ccccc8)c7cc65)ccc4c3)n2)cc1]

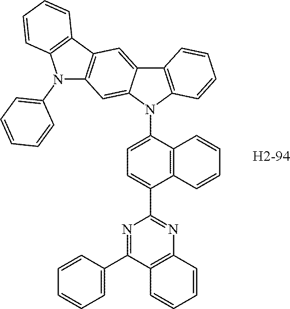 [SMILES: c1ccc(-c2nc(-c3ccc(-n4c5ccccc5c5cc6c7ccccc7n(-c7ccccc7)c6cc54)c4ccccc34)nc3ccccc23)cc1]

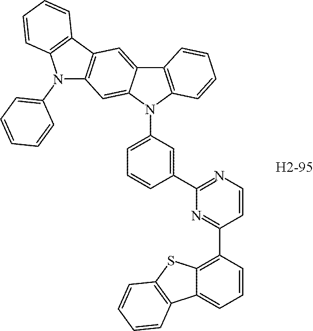 [SMILES: c1ccc(-n2c3ccccc3c3cc4c5ccccc5n(-c5cccc(-c6nccc(-c7cccc8c7sc7ccccc78)n6)c5)c4cc32)cc1]

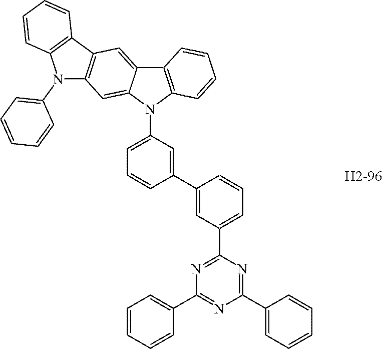 [SMILES: c1ccc(-c2nc(-c3ccccc3)nc(-c3cccc(-c4cccc(-n5c6ccccc6c6cc7c8ccccc8n(-c8ccccc8)c7cc65)c4)c3)n2)cc1]

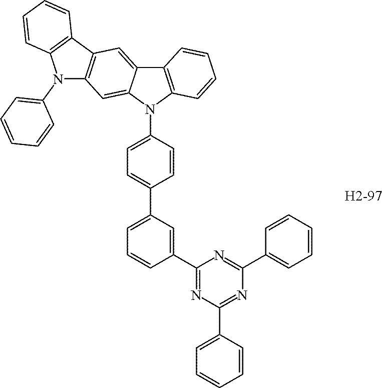 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1]

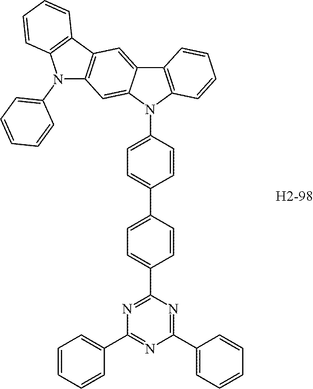 [SMILES: c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1]

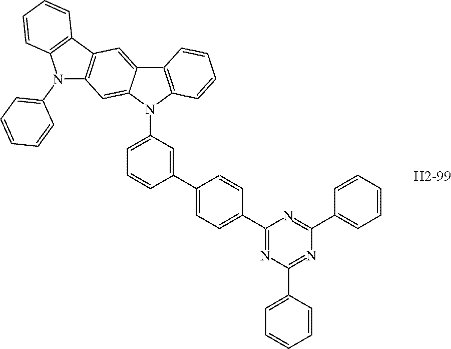 [SMILES: c1ccc(-c2nc(-c3ccccc3)nc(-c3ccc(-c4cccc(-n5c6ccccc6c6cc7c8ccccc8n(-c8ccccc8)c7cc65)c4)cc3)n2)cc1]

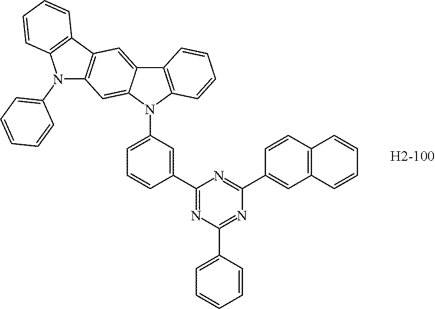 [SMILES: c1ccc(-c2nc(-c3cccc(-n4c5ccccc5c5cc6c7ccccc7n(-c7ccccc7)c6cc54)c3)nc(-c3ccc4ccccc4c3)n2)cc1]

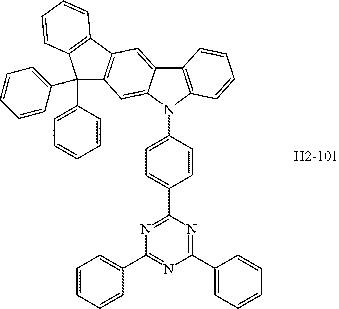 [SMILES: c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1]

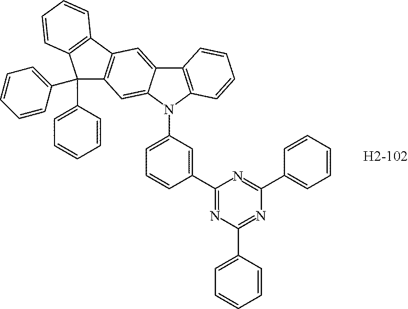 [SMILES: c1ccc(-c2nc(-c3ccccc3)nc(-c3cccc(-n4c5ccccc5c5cc6c(cc54)C(c4ccccc4)(c4ccccc4)c4ccccc4-6)c3)n2)cc1]

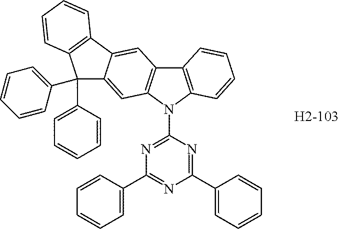 [SMILES: c1ccc(-c2nc(-c3ccccc3)nc(-n3c4ccccc4c4cc5c(cc43)C(c3ccccc3)(c3ccccc3)c3ccccc3-5)n2)cc1]

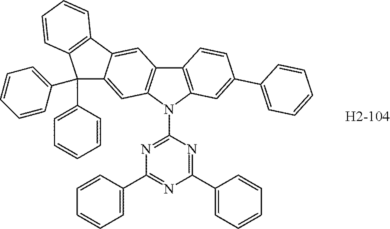 [SMILES: c1ccc(-c2ccc3c4cc5c(cc4n(-c4nc(-c6ccccc6)nc(-c6ccccc6)n4)c3c2)C(c2ccccc2)(c2ccccc2)c2ccccc2-5)cc1]

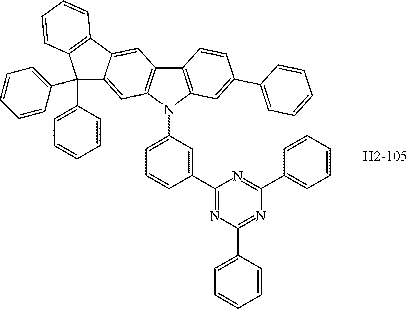 [SMILES: c1ccc(-c2ccc3c4cc5c(cc4n(-c4cccc(-c6nc(-c7ccccc7)nc(-c7ccccc7)n6)c4)c3c2)C(c2ccccc2)(c2ccccc2)c2ccccc2-5)cc1]

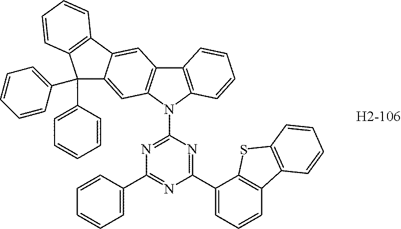 [SMILES: c1ccc(-c2nc(-c3cccc4c3sc3ccccc34)nc(-n3c4ccccc4c4cc5c(cc43)C(c3ccccc3)(c3ccccc3)c3ccccc3-5)n2)cc1]

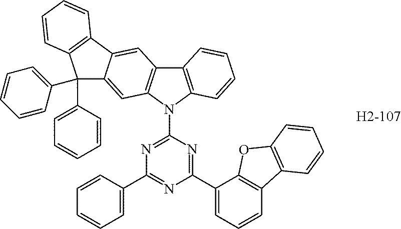 [SMILES: c1ccc(-c2nc(-c3cccc4c3oc3ccccc34)nc(-n3c4ccccc4c4cc5c(cc43)C(c3ccccc3)(c3ccccc3)c3ccccc3-5)n2)cc1]

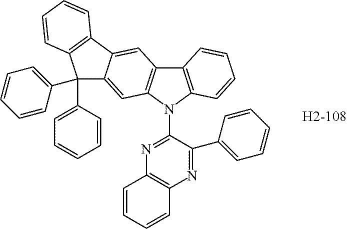 [SMILES: c1ccc(-c2nc3ccccc3nc2-n2c3ccccc3c3cc4c(cc32)C(c2ccccc2)(c2ccccc2)c2ccccc2-4)cc1]

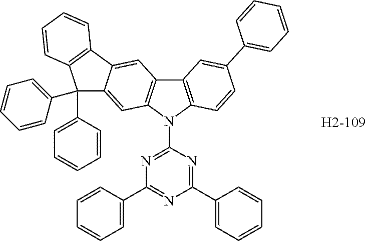 [SMILES: c1ccc(-c2ccc3c(c2)c2cc4c(cc2n3-c2nc(-c3ccccc3)nc(-c3ccccc3)n2)C(c2ccccc2)(c2ccccc2)c2ccccc2-4)cc1]

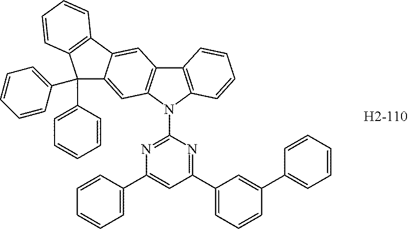 [SMILES: c1ccc(-c2cccc(-c3cc(-c4ccccc4)nc(-n4c5ccccc5c5cc6c(cc54)C(c4ccccc4)(c4ccccc4)c4ccccc4-6)n3)c2)cc1]

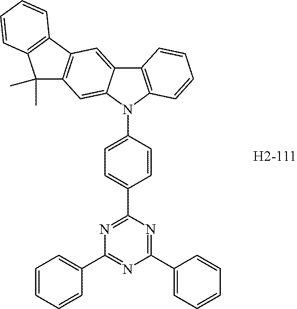 [SMILES: CC1(C)c2ccccc2-c2cc3c4ccccc4n(-c4ccc(-c5nc(-c6ccccc6)nc(-c6ccccc6)n5)cc4)c3cc21]

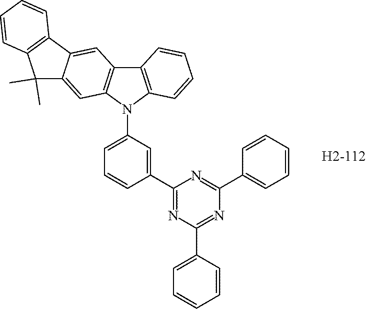 [SMILES: CC1(C)c2ccccc2-c2cc3c4ccccc4n(-c4cccc(-c5nc(-c6ccccc6)nc(-c6ccccc6)n5)c4)c3cc21]

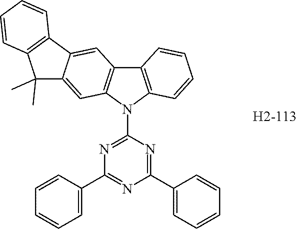 [SMILES: CC1(C)c2ccccc2-c2cc3c4ccccc4n(-c4nc(-c5ccccc5)nc(-c5ccccc5)n4)c3cc21]

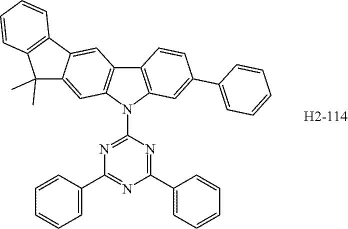 [SMILES: CC1(C)c2ccccc2-c2cc3c4ccc(-c5ccccc5)cc4n(-c4nc(-c5ccccc5)nc(-c5ccccc5)n4)c3cc21]

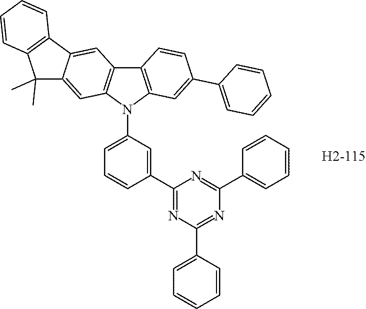 [SMILES: CC1(C)c2ccccc2-c2cc3c4ccc(-c5ccccc5)cc4n(-c4cccc(-c5nc(-c6ccccc6)nc(-c6ccccc6)n5)c4)c3cc21]

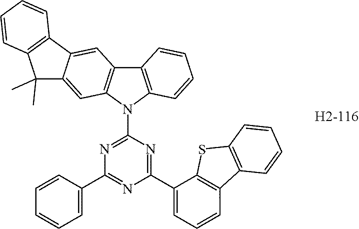 [SMILES: CC1(C)c2ccccc2-c2cc3c4ccccc4n(-c4nc(-c5ccccc5)nc(-c5cccc6c5sc5ccccc56)n4)c3cc21]

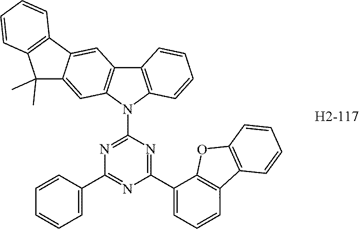 [SMILES: CC1(C)c2ccccc2-c2cc3c4ccccc4n(-c4nc(-c5ccccc5)nc(-c5cccc6c5oc5ccccc56)n4)c3cc21]

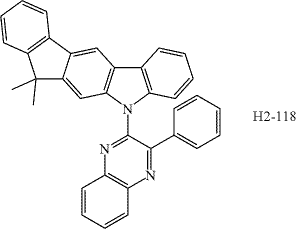 [SMILES: CC1(C)c2ccccc2-c2cc3c4ccccc4n(-c4nc5ccccc5nc4-c4ccccc4)c3cc21]

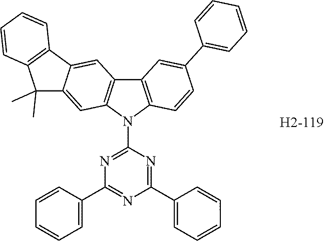 [SMILES: CC1(C)c2ccccc2-c2cc3c4cc(-c5ccccc5)ccc4n(-c4nc(-c5ccccc5)nc(-c5ccccc5)n4)c3cc21]

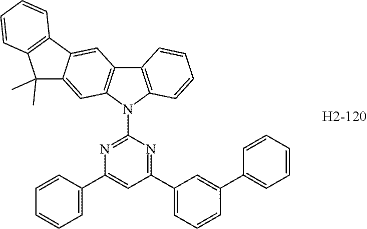 [SMILES: CC1(C)c2ccccc2-c2cc3c4ccccc4n(-c4nc(-c5ccccc5)cc(-c5cccc(-c6ccccc6)c5)n4)c3cc21]

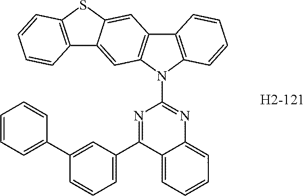 [SMILES: c1ccc(-c2cccc(-c3nc(-n4c5ccccc5c5cc6sc7ccccc7c6cc54)nc4ccccc34)c2)cc1]

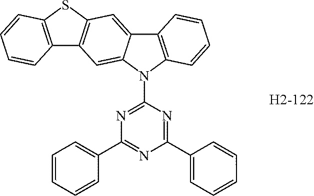 [SMILES: c1ccc(-c2nc(-c3ccccc3)nc(-n3c4ccccc4c4cc5sc6ccccc6c5cc43)n2)cc1]

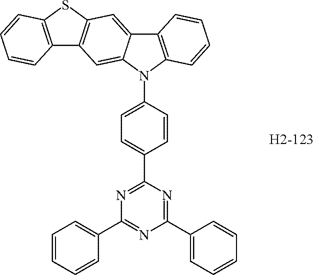 [SMILES: c1ccc(-c2nc(-c3ccccc3)nc(-c3ccc(-n4c5ccccc5c5cc6sc7ccccc7c6cc54)cc3)n2)cc1]

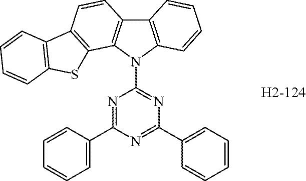 [SMILES: c1ccc(-c2nc(-c3ccccc3)nc(-n3c4ccccc4c4ccc5c6ccccc6sc5c43)n2)cc1]

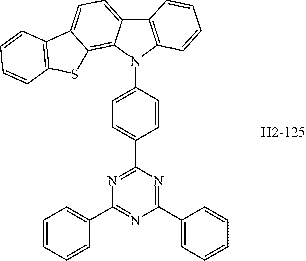 [SMILES: c1ccc(-c2nc(-c3ccccc3)nc(-c3ccc(-n4c5ccccc5c5ccc6c7ccccc7sc6c54)cc3)n2)cc1]

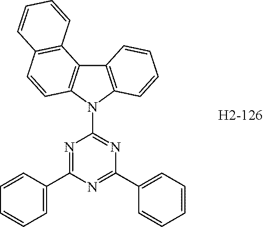 [SMILES: c1ccc(-c2nc(-c3ccccc3)nc(-n3c4ccccc4c4c5ccccc5ccc43)n2)cc1]

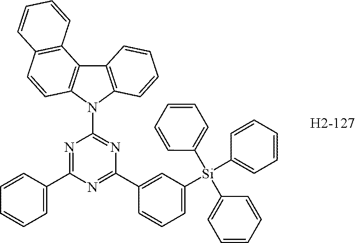 [SMILES: c1ccc(-c2nc(-c3cccc([Si](c4ccccc4)(c4ccccc4)c4ccccc4)c3)nc(-n3c4ccccc4c4c5ccccc5ccc43)n2)cc1]

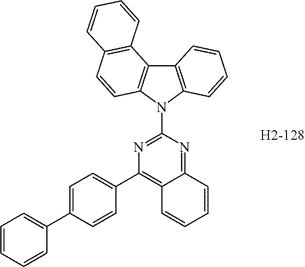 [SMILES: c1ccc(-c2ccc(-c3nc(-n4c5ccccc5c5c6ccccc6ccc54)nc4ccccc34)cc2)cc1]

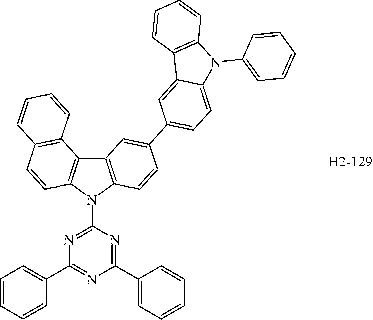 [SMILES: c1ccc(-c2nc(-c3ccccc3)nc(-n3c4ccc(-c5ccc6c(c5)c5ccccc5n6-c5ccccc5)cc4c4c5ccccc5ccc43)n2)cc1]

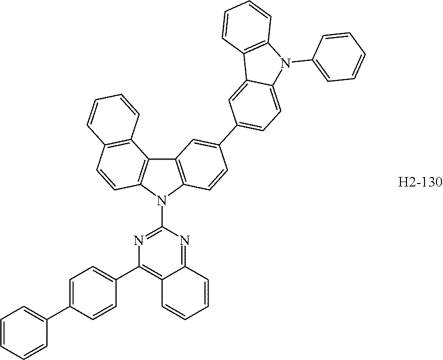 [SMILES: c1ccc(-c2ccc(-c3nc(-n4c5ccc(-c6ccc7c(c6)c6ccccc6n7-c6ccccc6)cc5c5c6ccccc6ccc54)nc4ccccc34)cc2)cc1]

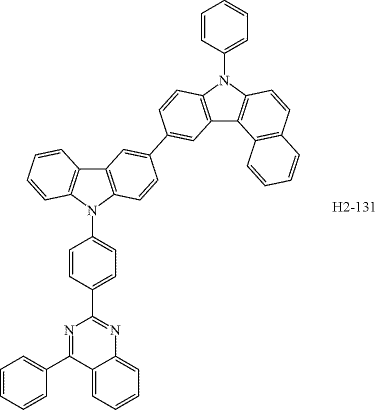 [SMILES: c1ccc(-c2nc(-c3ccc(-n4c5ccccc5c5cc(-c6ccc7c(c6)c6c8ccccc8ccc6n7-c6ccccc6)ccc54)cc3)nc3ccccc23)cc1]

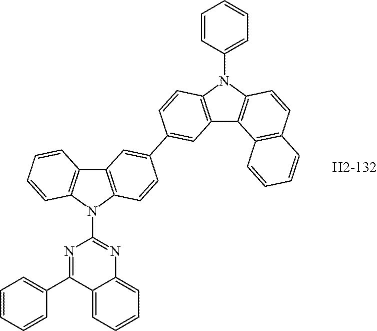 [SMILES: c1ccc(-c2nc(-n3c4ccccc4c4cc(-c5ccc6c(c5)c5c7ccccc7ccc5n6-c5ccccc5)ccc43)nc3ccccc23)cc1]

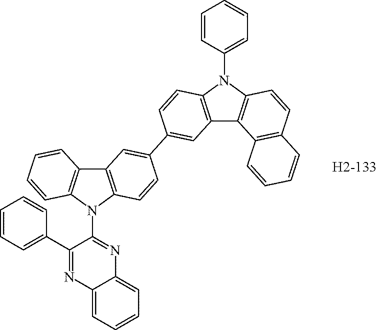 [SMILES: c1ccc(-c2nc3ccccc3nc2-n2c3ccccc3c3cc(-c4ccc5c(c4)c4c6ccccc6ccc4n5-c4ccccc4)ccc32)cc1]

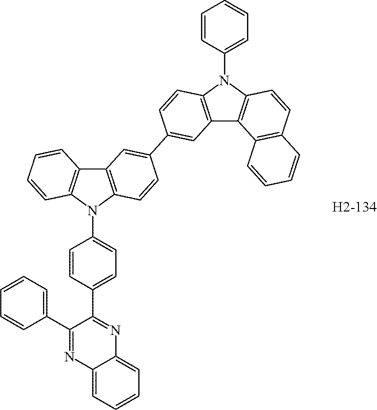 [SMILES: c1ccc(-c2nc3ccccc3nc2-c2ccc(-n3c4ccccc4c4cc(-c5ccc6c(c5)c5c7ccccc7ccc5n6-c5ccccc5)ccc43)cc2)cc1]

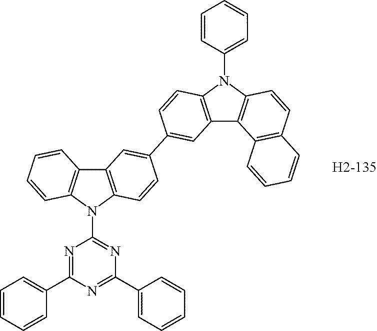 [SMILES: c1ccc(-c2nc(-c3ccccc3)nc(-n3c4ccccc4c4cc(-c5ccc6c(c5)c5c7ccccc7ccc5n6-c5ccccc5)ccc43)n2)cc1]

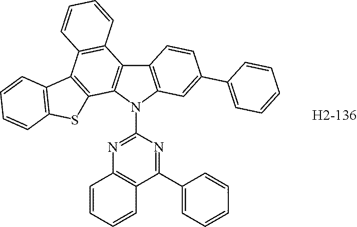 [SMILES: c1ccc(-c2ccc3c4c5ccccc5c5c6ccccc6sc5c4n(-c4nc(-c5ccccc5)c5ccccc5n4)c3c2)cc1]

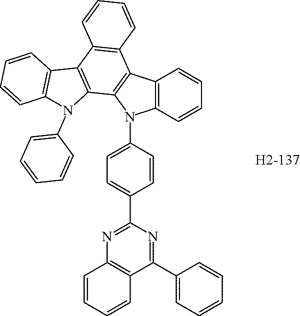 [SMILES: c1ccc(-c2nc(-c3ccc(-n4c5ccccc5c5c6ccccc6c6c7ccccc7n(-c7ccccc7)c6c54)cc3)nc3ccccc23)cc1]

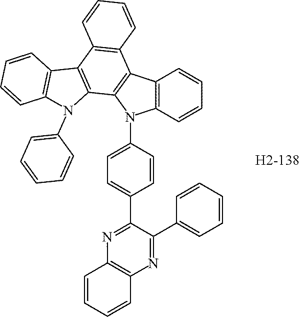 [SMILES: c1ccc(-c2nc3ccccc3nc2-c2ccc(-n3c4ccccc4c4c5ccccc5c5c6ccccc6n(-c6ccccc6)c5c43)cc2)cc1]

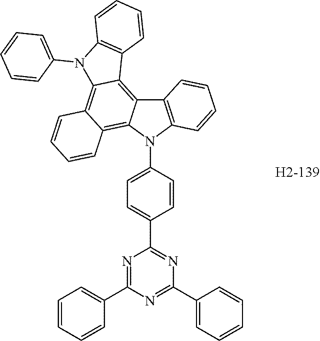 [SMILES: c1ccc(-c2nc(-c3ccccc3)nc(-c3ccc(-n4c5ccccc5c5c6c7ccccc7n(-c7ccccc7)c6c6ccccc6c54)cc3)n2)cc1]

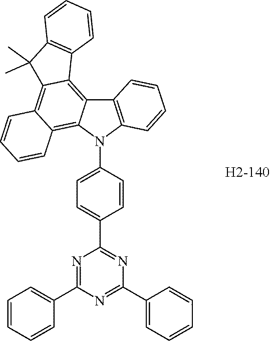 [SMILES: CC1(C)c2ccccc2-c2c1c1ccccc1c1c2c2ccccc2n1-c1ccc(-c2nc(-c3ccccc3)nc(-c3ccccc3)n2)cc1]

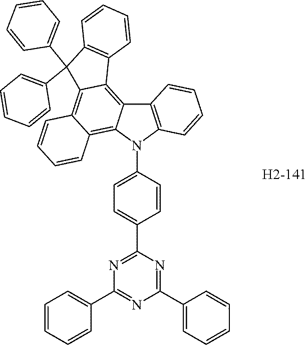 [SMILES: c1ccc(-c2nc(-c3ccccc3)nc(-c3ccc(-n4c5ccccc5c5c6c(c7ccccc7c54)C(c4ccccc4)(c4ccccc4)c4ccccc4-6)cc3)n2)cc1]

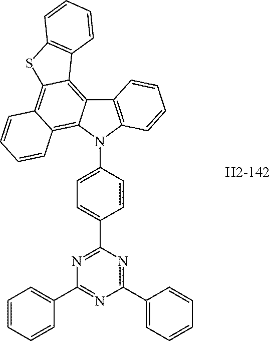 [SMILES: c1ccc(-c2nc(-c3ccccc3)nc(-c3ccc(-n4c5ccccc5c5c6c7ccccc7sc6c6ccccc6c54)cc3)n2)cc1]

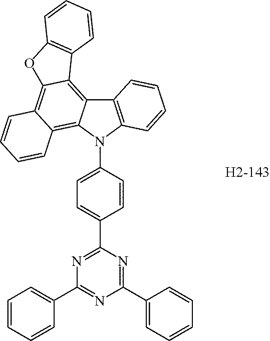 [SMILES: c1ccc(-c2nc(-c3ccccc3)nc(-c3ccc(-n4c5ccccc5c5c6c7ccccc7oc6c6ccccc6c54)cc3)n2)cc1]

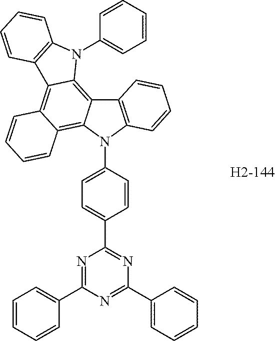 [SMILES: c1ccc(-c2nc(-c3ccccc3)nc(-c3ccc(-n4c5ccccc5c5c4c4ccccc4c4c6ccccc6n(-c6ccccc6)c45)cc3)n2)cc1]

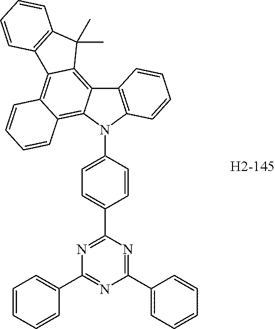 [SMILES: CC1(C)c2ccccc2-c2c1c1c3ccccc3n(-c3ccc(-c4nc(-c5ccccc5)nc(-c5ccccc5)n4)cc3)c1c1ccccc21]

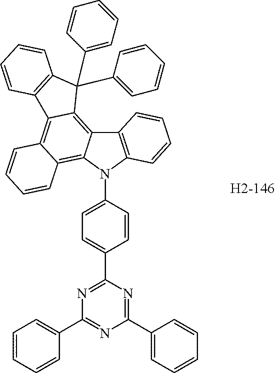 [SMILES: c1ccc(-c2nc(-c3ccccc3)nc(-c3ccc(-n4c5ccccc5c5c6c(c7ccccc7c54)-c4ccccc4C6(c4ccccc4)c4ccccc4)cc3)n2)cc1]

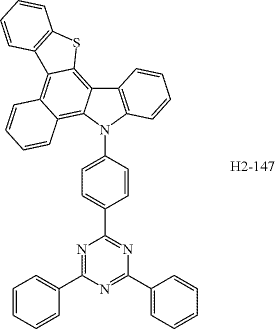 [SMILES: c1ccc(-c2nc(-c3ccccc3)nc(-c3ccc(-n4c5ccccc5c5c6sc7ccccc7c6c6ccccc6c54)cc3)n2)cc1]

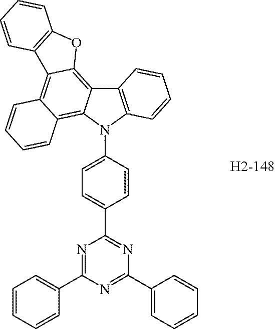 [SMILES: c1ccc(-c2nc(-c3ccccc3)nc(-c3ccc(-n4c5ccccc5c5c6oc7ccccc7c6c6ccccc6c54)cc3)n2)cc1]

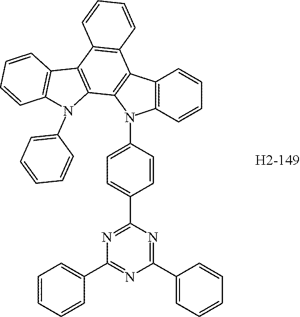 [SMILES: c1ccc(-c2nc(-c3ccccc3)nc(-c3ccc(-n4c5ccccc5c5c6ccccc6c6c7ccccc7n(-c7ccccc7)c6c54)cc3)n2)cc1]

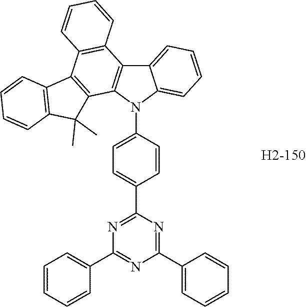 [SMILES: CC1(C)c2ccccc2-c2c1c1c(c3ccccc23)c2ccccc2n1-c1ccc(-c2nc(-c3ccccc3)nc(-c3ccccc3)n2)cc1]

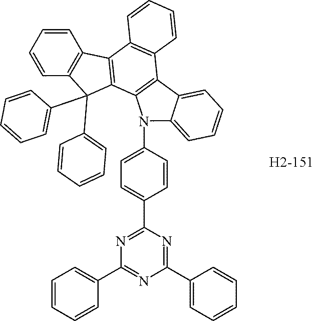 [SMILES: c1ccc(-c2nc(-c3ccccc3)nc(-c3ccc(-n4c5ccccc5c5c6ccccc6c6c(c54)C(c4ccccc4)(c4ccccc4)c4ccccc4-6)cc3)n2)cc1]

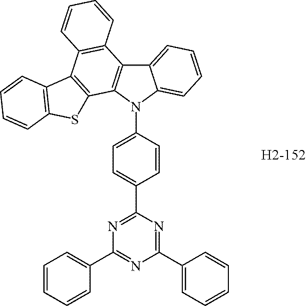 [SMILES: c1ccc(-c2nc(-c3ccccc3)nc(-c3ccc(-n4c5ccccc5c5c6ccccc6c6c7ccccc7sc6c54)cc3)n2)cc1]

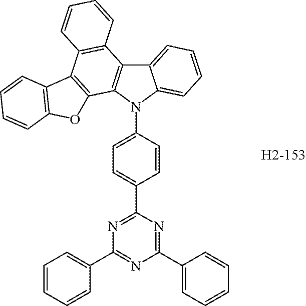 [SMILES: c1ccc(-c2nc(-c3ccccc3)nc(-c3ccc(-n4c5ccccc5c5c6ccccc6c6c7ccccc7oc6c54)cc3)n2)cc1]

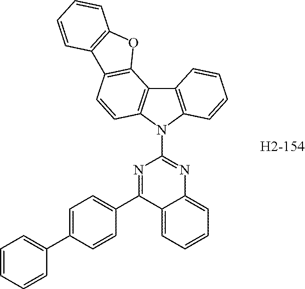 [SMILES: c1ccc(-c2ccc(-c3nc(-n4c5ccccc5c5c6oc7ccccc7c6ccc54)nc4ccccc34)cc2)cc1]

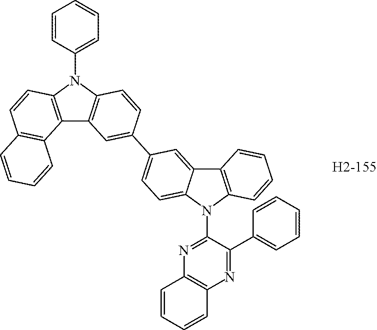 [SMILES: c1ccc(-c2nc3ccccc3nc2-n2c3ccccc3c3cc(-c4ccc5c(c4)c4c6ccccc6ccc4n5-c4ccccc4)ccc32)cc1]

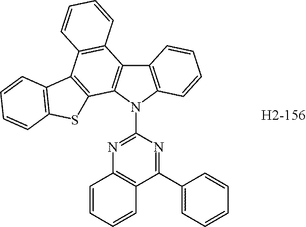 [SMILES: c1ccc(-c2nc(-n3c4ccccc4c4c5ccccc5c5c6ccccc6sc5c43)nc3ccccc23)cc1]

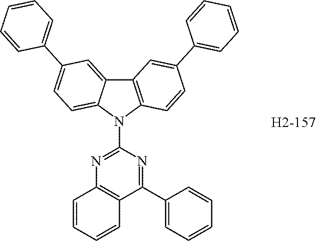 [SMILES: c1ccc(-c2ccc3c(c2)c2cc(-c4ccccc4)ccc2n3-c2nc(-c3ccccc3)c3ccccc3n2)cc1]

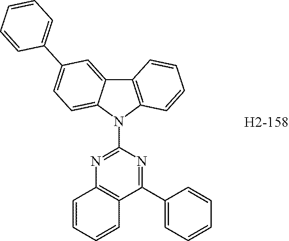 [SMILES: c1ccc(-c2ccc3c(c2)c2ccccc2n3-c2nc(-c3ccccc3)c3ccccc3n2)cc1]

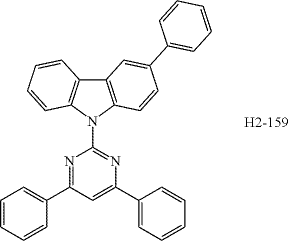 [SMILES: c1ccc(-c2ccc3c(c2)c2ccccc2n3-c2nc(-c3ccccc3)cc(-c3ccccc3)n2)cc1]

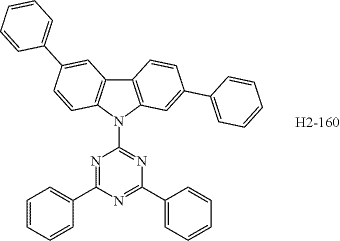 [SMILES: c1ccc(-c2ccc3c(c2)c2ccc(-c4ccccc4)cc2n3-c2nc(-c3ccccc3)nc(-c3ccccc3)n2)cc1]

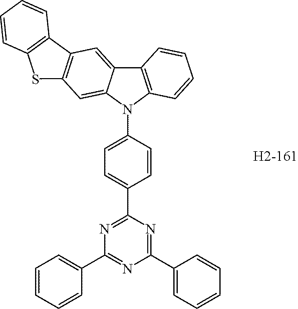 [SMILES: c1ccc(-c2nc(-c3ccccc3)nc(-c3ccc(-n4c5ccccc5c5cc6c(cc54)sc4ccccc46)cc3)n2)cc1]

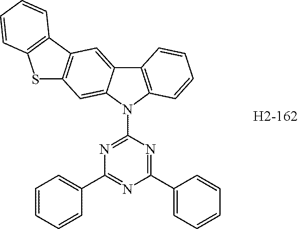 [SMILES: c1ccc(-c2nc(-c3ccccc3)nc(-n3c4ccccc4c4cc5c(cc43)sc3ccccc35)n2)cc1]

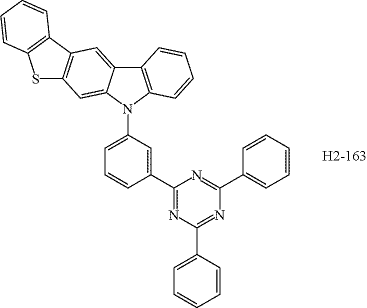 [SMILES: c1ccc(-c2nc(-c3ccccc3)nc(-c3cccc(-n4c5ccccc5c5cc6c(cc54)sc4ccccc46)c3)n2)cc1]

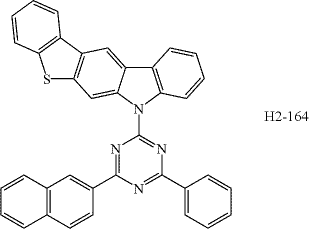 [SMILES: c1ccc(-c2nc(-c3ccc4ccccc4c3)nc(-n3c4ccccc4c4cc5c(cc43)sc3ccccc35)n2)cc1]

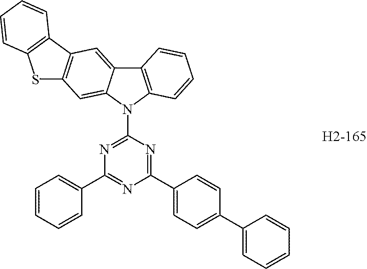 [SMILES: c1ccc(-c2ccc(-c3nc(-c4ccccc4)nc(-n4c5ccccc5c5cc6c(cc54)sc4ccccc46)n3)cc2)cc1]

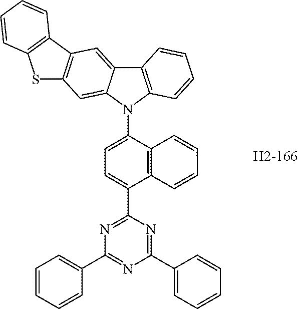 [SMILES: c1ccc(-c2nc(-c3ccccc3)nc(-c3ccc(-n4c5ccccc5c5cc6c(cc54)sc4ccccc46)c4ccccc34)n2)cc1]

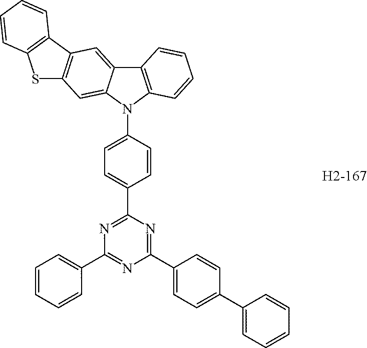 [SMILES: c1ccc(-c2ccc(-c3nc(-c4ccccc4)nc(-c4ccc(-n5c6ccccc6c6cc7c(cc65)sc5ccccc57)cc4)n3)cc2)cc1]

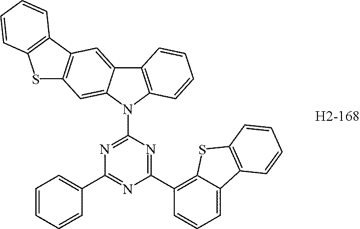 [SMILES: c1ccc(-c2nc(-c3cccc4c3sc3ccccc34)nc(-n3c4ccccc4c4cc5c(cc43)sc3ccccc35)n2)cc1]

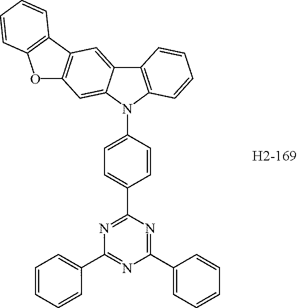 [SMILES: c1ccc(-c2nc(-c3ccccc3)nc(-c3ccc(-n4c5ccccc5c5cc6c(cc54)oc4ccccc46)cc3)n2)cc1]

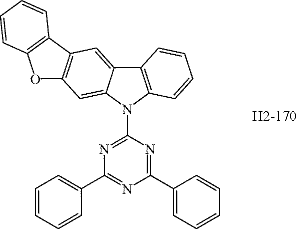 [SMILES: c1ccc(-c2nc(-c3ccccc3)nc(-n3c4ccccc4c4cc5c(cc43)oc3ccccc35)n2)cc1]

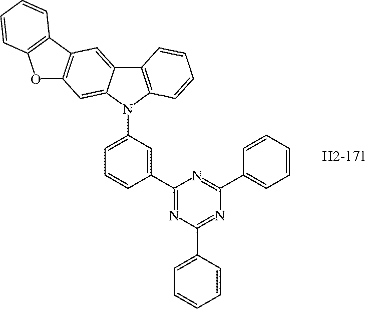 [SMILES: c1ccc(-c2nc(-c3ccccc3)nc(-c3cccc(-n4c5ccccc5c5cc6c(cc54)oc4ccccc46)c3)n2)cc1]

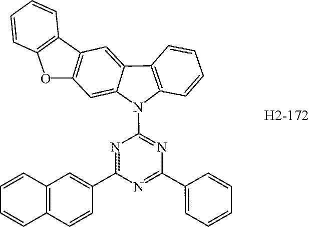 [SMILES: c1ccc(-c2nc(-c3ccc4ccccc4c3)nc(-n3c4ccccc4c4cc5c(cc43)oc3ccccc35)n2)cc1]

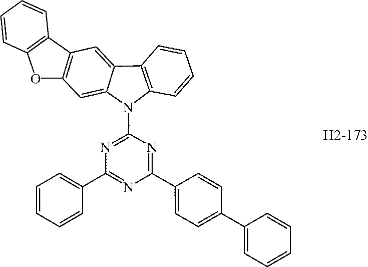 [SMILES: c1ccc(-c2ccc(-c3nc(-c4ccccc4)nc(-n4c5ccccc5c5cc6c(cc54)oc4ccccc46)n3)cc2)cc1]

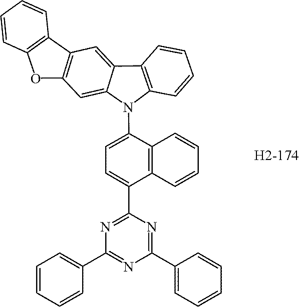 [SMILES: c1ccc(-c2nc(-c3ccccc3)nc(-c3ccc(-n4c5ccccc5c5cc6c(cc54)oc4ccccc46)c4ccccc34)n2)cc1]

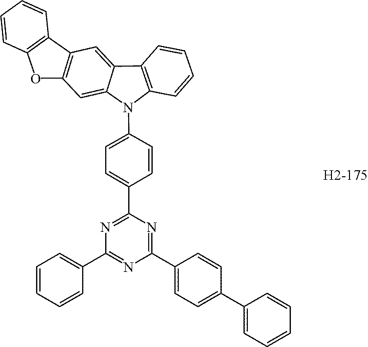 [SMILES: c1ccc(-c2ccc(-c3nc(-c4ccccc4)nc(-c4ccc(-n5c6ccccc6c6cc7c(cc65)oc5ccccc57)cc4)n3)cc2)cc1]

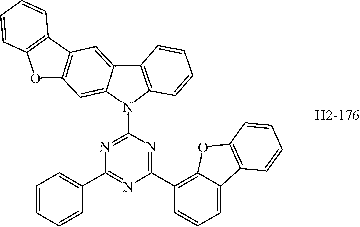 [SMILES: c1ccc(-c2nc(-c3cccc4c3oc3ccccc34)nc(-n3c4ccccc4c4cc5c(cc43)oc3ccccc35)n2)cc1]

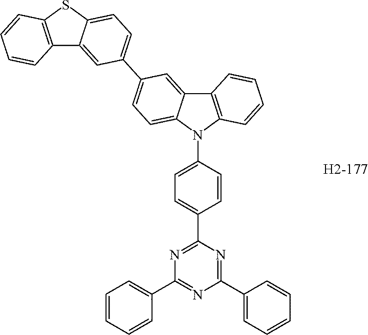 [SMILES: c1ccc(-c2nc(-c3ccccc3)nc(-c3ccc(-n4c5ccccc5c5cc(-c6ccc7sc8ccccc8c7c6)ccc54)cc3)n2)cc1]

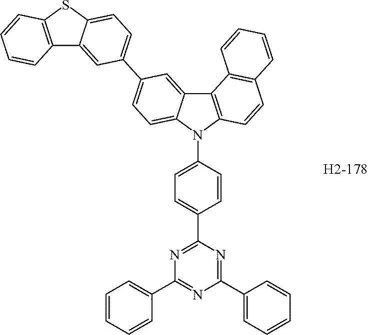 [SMILES: c1ccc(-c2nc(-c3ccccc3)nc(-c3ccc(-n4c5ccc(-c6ccc7sc8ccccc8c7c6)cc5c5c6ccccc6ccc54)cc3)n2)cc1]

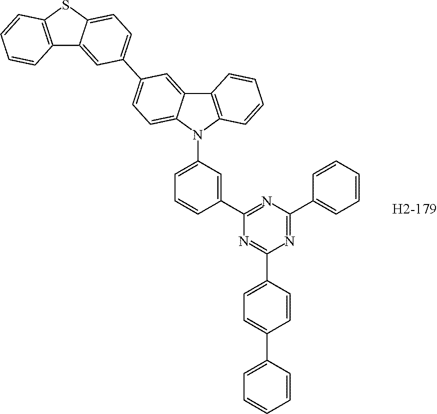 [SMILES: c1ccc(-c2ccc(-c3nc(-c4ccccc4)nc(-c4cccc(-n5c6ccccc6c6cc(-c7ccc8sc9ccccc9c8c7)ccc65)c4)n3)cc2)cc1]

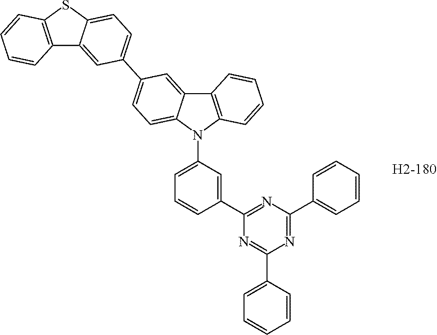 [SMILES: c1ccc(-c2nc(-c3ccccc3)nc(-c3cccc(-n4c5ccccc5c5cc(-c6ccc7sc8ccccc8c7c6)ccc54)c3)n2)cc1]

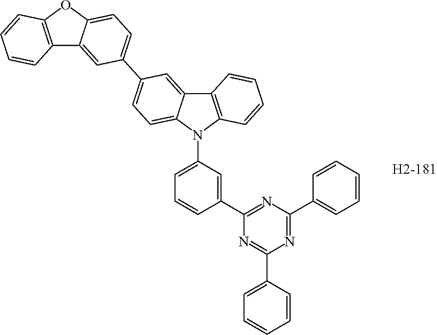 [SMILES: c1ccc(-c2nc(-c3ccccc3)nc(-c3cccc(-n4c5ccccc5c5cc(-c6ccc7oc8ccccc8c7c6)ccc54)c3)n2)cc1]

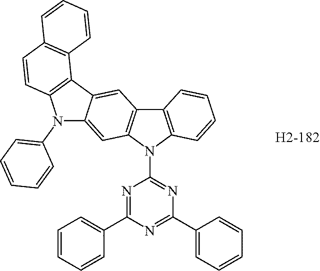 [SMILES: c1ccc(-c2nc(-c3ccccc3)nc(-n3c4ccccc4c4cc5c6c7ccccc7ccc6n(-c6ccccc6)c5cc43)n2)cc1]

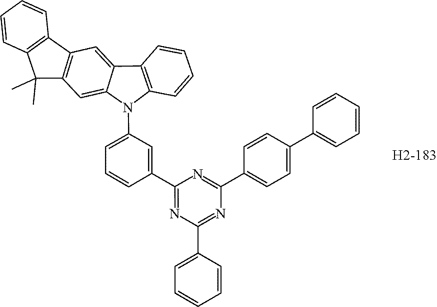 [SMILES: CC1(C)c2ccccc2-c2cc3c4ccccc4n(-c4cccc(-c5nc(-c6ccccc6)nc(-c6ccc(-c7ccccc7)cc6)n5)c4)c3cc21]

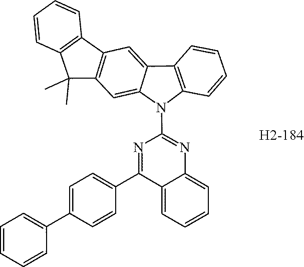 [SMILES: CC1(C)c2ccccc2-c2cc3c4ccccc4n(-c4nc(-c5ccc(-c6ccccc6)cc5)c5ccccc5n4)c3cc21]

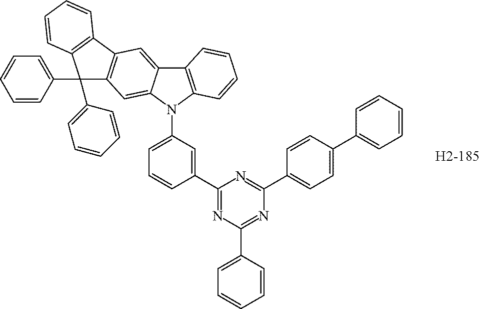 [SMILES: c1ccc(-c2ccc(-c3nc(-c4ccccc4)nc(-c4cccc(-n5c6ccccc6c6cc7c(cc65)C(c5ccccc5)(c5ccccc5)c5ccccc5-7)c4)n3)cc2)cc1]

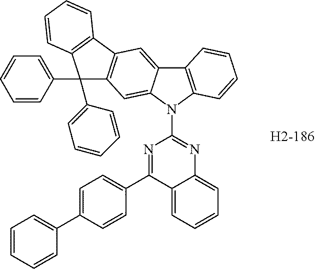 [SMILES: c1ccc(-c2ccc(-c3nc(-n4c5ccccc5c5cc6c(cc54)C(c4ccccc4)(c4ccccc4)c4ccccc4-6)nc4ccccc34)cc2)cc1]

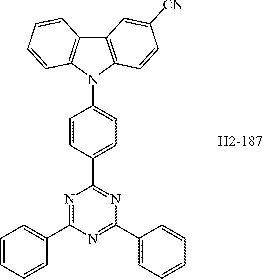 [SMILES: N#Cc1ccc2c(c1)c1ccccc1n2-c1ccc(-c2nc(-c3ccccc3)nc(-c3ccccc3)n2)cc1]

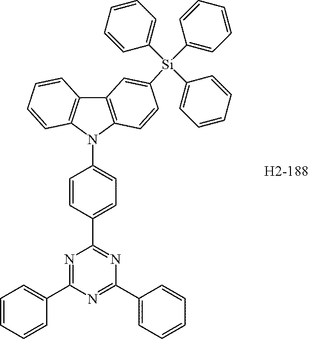 [SMILES: c1ccc(-c2nc(-c3ccccc3)nc(-c3ccc(-n4c5ccccc5c5cc([Si](c6ccccc6)(c6ccccc6)c6ccccc6)ccc54)cc3)n2)cc1]

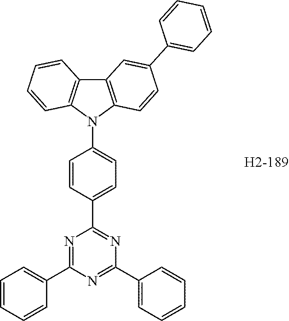 [SMILES: c1ccc(-c2ccc3c(c2)c2ccccc2n3-c2ccc(-c3nc(-c4ccccc4)nc(-c4ccccc4)n3)cc2)cc1]

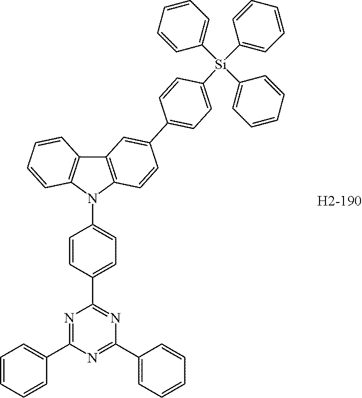 [SMILES: c1ccc(-c2nc(-c3ccccc3)nc(-c3ccc(-n4c5ccccc5c5cc(-c6ccc([Si](c7ccccc7)(c7ccccc7)c7ccccc7)cc6)ccc54)cc3)n2)cc1]

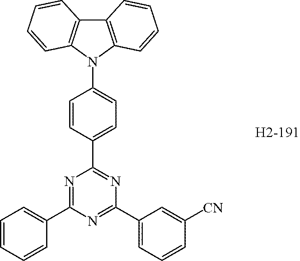 [SMILES: N#Cc1cccc(-c2nc(-c3ccccc3)nc(-c3ccc(-n4c5ccccc5c5ccccc54)cc3)n2)c1]

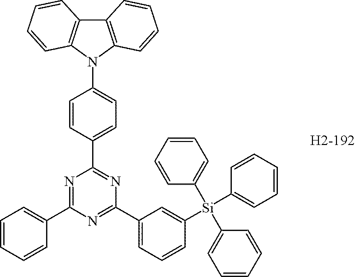 [SMILES: c1ccc(-c2nc(-c3ccc(-n4c5ccccc5c5ccccc54)cc3)nc(-c3cccc([Si](c4ccccc4)(c4ccccc4)c4ccccc4)c3)n2)cc1]

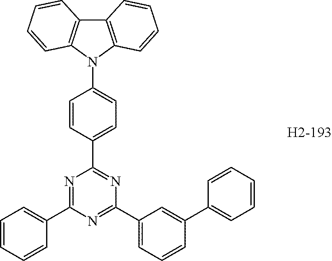 [SMILES: c1ccc(-c2cccc(-c3nc(-c4ccccc4)nc(-c4ccc(-n5c6ccccc6c6ccccc65)cc4)n3)c2)cc1]

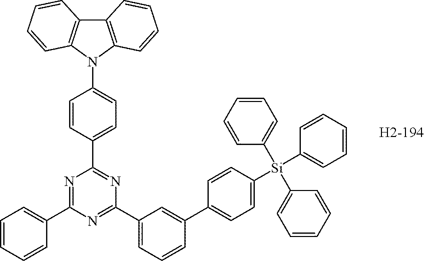 [SMILES: c1ccc(-c2nc(-c3ccc(-n4c5ccccc5c5ccccc54)cc3)nc(-c3cccc(-c4ccc([Si](c5ccccc5)(c5ccccc5)c5ccccc5)cc4)c3)n2)cc1]

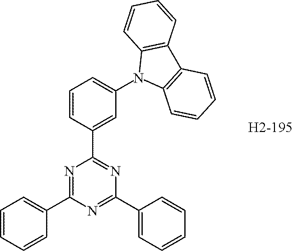 [SMILES: c1ccc(-c2nc(-c3ccccc3)nc(-c3cccc(-n4c5ccccc5c5ccccc54)c3)n2)cc1]

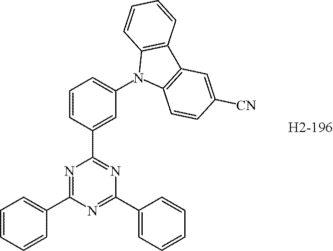 [SMILES: N#Cc1ccc2c(c1)c1ccccc1n2-c1cccc(-c2nc(-c3ccccc3)nc(-c3ccccc3)n2)c1]

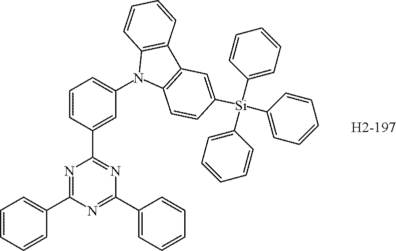 [SMILES: c1ccc(-c2nc(-c3ccccc3)nc(-c3cccc(-n4c5ccccc5c5cc([Si](c6ccccc6)(c6ccccc6)c6ccccc6)ccc54)c3)n2)cc1]

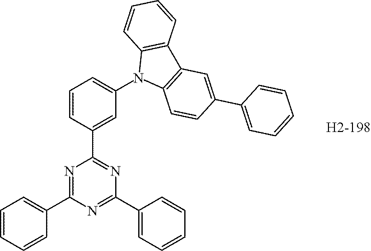 [SMILES: c1ccc(-c2ccc3c(c2)c2ccccc2n3-c2cccc(-c3nc(-c4ccccc4)nc(-c4ccccc4)n3)c2)cc1]

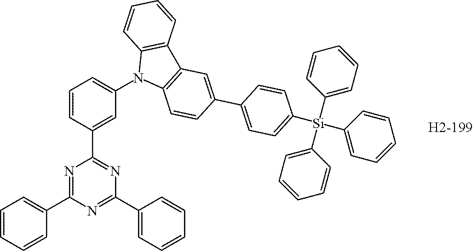 [SMILES: c1ccc(-c2nc(-c3ccccc3)nc(-c3cccc(-n4c5ccccc5c5cc(-c6ccc([Si](c7ccccc7)(c7ccccc7)c7ccccc7)cc6)ccc54)c3)n2)cc1]

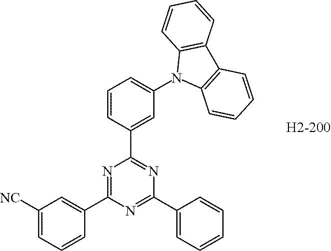 [SMILES: N#Cc1cccc(-c2nc(-c3ccccc3)nc(-c3cccc(-n4c5ccccc5c5ccccc54)c3)n2)c1]

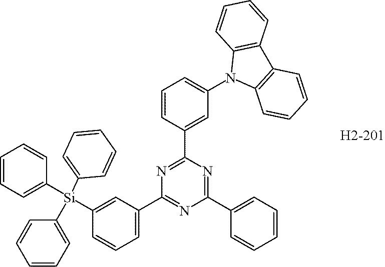 [SMILES: c1ccc(-c2nc(-c3cccc(-n4c5ccccc5c5ccccc54)c3)nc(-c3cccc([Si](c4ccccc4)(c4ccccc4)c4ccccc4)c3)n2)cc1]

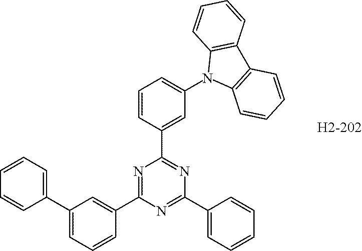 [SMILES: c1ccc(-c2cccc(-c3nc(-c4ccccc4)nc(-c4cccc(-n5c6ccccc6c6ccccc65)c4)n3)c2)cc1]

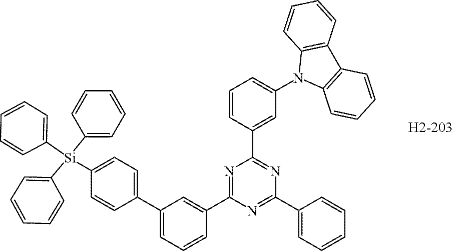 [SMILES: c1ccc(-c2nc(-c3cccc(-c4ccc([Si](c5ccccc5)(c5ccccc5)c5ccccc5)cc4)c3)nc(-c3cccc(-n4c5ccccc5c5ccccc54)c3)n2)cc1]

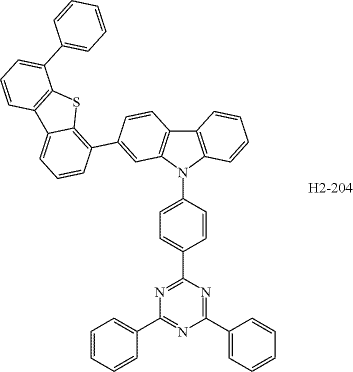 [SMILES: c1ccc(-c2nc(-c3ccccc3)nc(-c3ccc(-n4c5ccccc5c5ccc(-c6cccc7c6sc6c(-c8ccccc8)cccc67)cc54)cc3)n2)cc1]

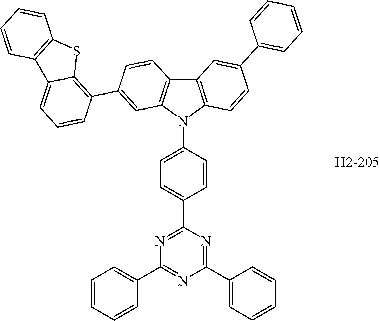 [SMILES: c1ccc(-c2ccc3c(c2)c2ccc(-c4cccc5c4sc4ccccc45)cc2n3-c2ccc(-c3nc(-c4ccccc4)nc(-c4ccccc4)n3)cc2)cc1]

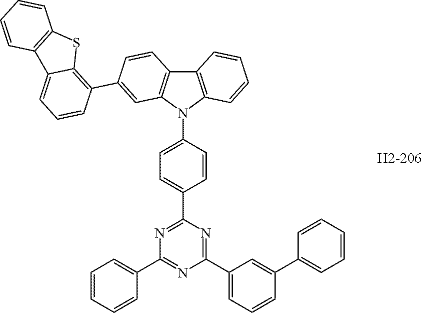 [SMILES: c1ccc(-c2cccc(-c3nc(-c4ccccc4)nc(-c4ccc(-n5c6ccccc6c6ccc(-c7cccc8c7sc7ccccc78)cc65)cc4)n3)c2)cc1]

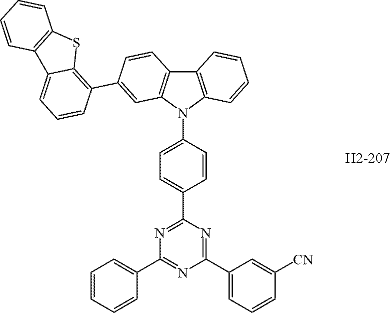 [SMILES: N#Cc1cccc(-c2nc(-c3ccccc3)nc(-c3ccc(-n4c5ccccc5c5ccc(-c6cccc7c6sc6ccccc67)cc54)cc3)n2)c1]

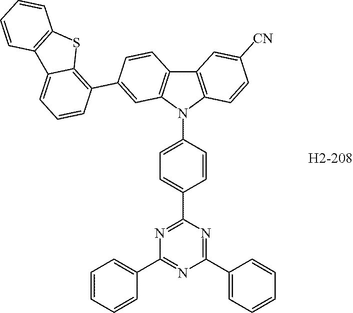 [SMILES: N#Cc1ccc2c(c1)c1ccc(-c3cccc4c3sc3ccccc34)cc1n2-c1ccc(-c2nc(-c3ccccc3)nc(-c3ccccc3)n2)cc1]

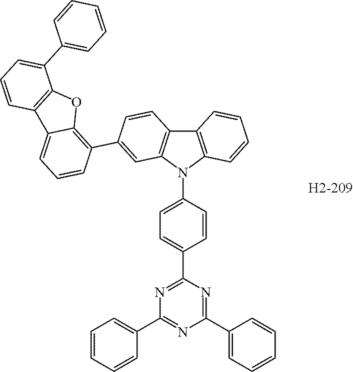 [SMILES: c1ccc(-c2nc(-c3ccccc3)nc(-c3ccc(-n4c5ccccc5c5ccc(-c6cccc7c6oc6c(-c8ccccc8)cccc67)cc54)cc3)n2)cc1]

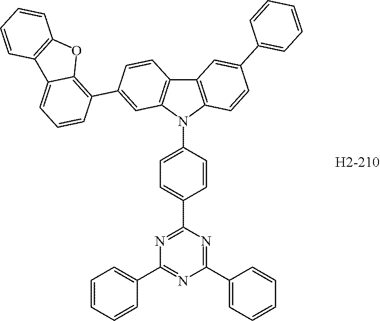 [SMILES: c1ccc(-c2ccc3c(c2)c2ccc(-c4cccc5c4oc4ccccc45)cc2n3-c2ccc(-c3nc(-c4ccccc4)nc(-c4ccccc4)n3)cc2)cc1]

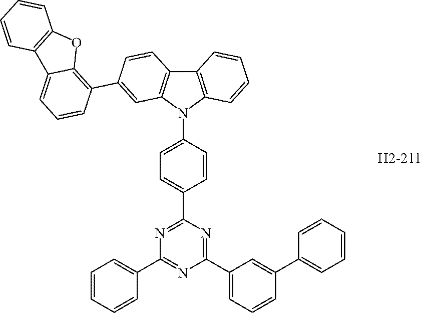 [SMILES: c1ccc(-c2cccc(-c3nc(-c4ccccc4)nc(-c4ccc(-n5c6ccccc6c6ccc(-c7cccc8c7oc7ccccc78)cc65)cc4)n3)c2)cc1]

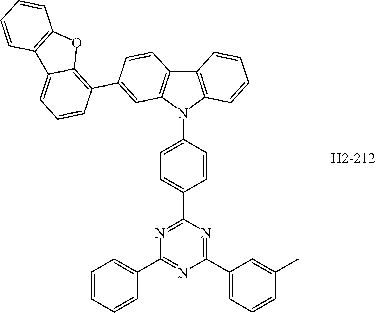 [SMILES: Cc1cccc(-c2nc(-c3ccccc3)nc(-c3ccc(-n4c5ccccc5c5ccc(-c6cccc7c6oc6ccccc67)cc54)cc3)n2)c1]

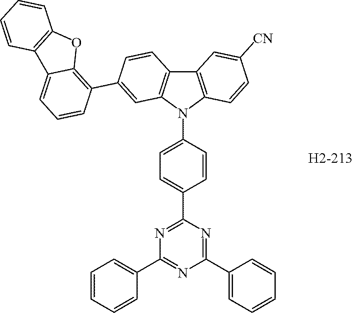 [SMILES: N#Cc1ccc2c(c1)c1ccc(-c3cccc4c3oc3ccccc34)cc1n2-c1ccc(-c2nc(-c3ccccc3)nc(-c3ccccc3)n2)cc1]

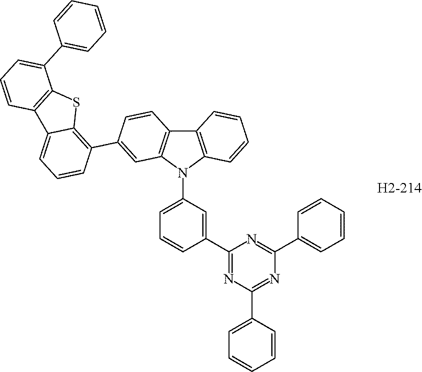 [SMILES: c1ccc(-c2nc(-c3ccccc3)nc(-c3cccc(-n4c5ccccc5c5ccc(-c6cccc7c6sc6c(-c8ccccc8)cccc67)cc54)c3)n2)cc1]

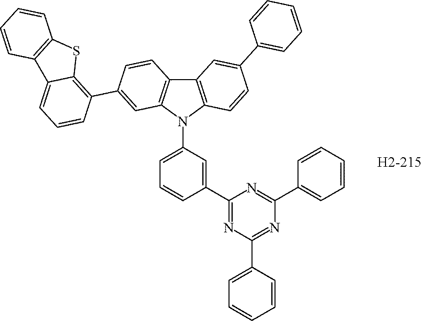 [SMILES: c1ccc(-c2ccc3c(c2)c2ccc(-c4cccc5c4sc4ccccc45)cc2n3-c2cccc(-c3nc(-c4ccccc4)nc(-c4ccccc4)n3)c2)cc1]

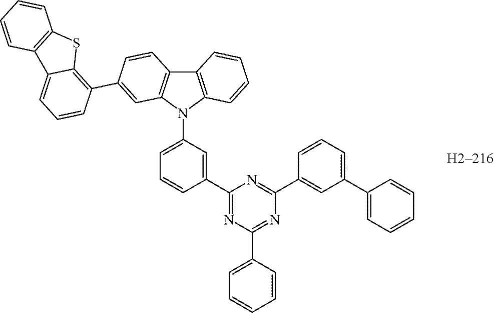 [SMILES: c1ccc(-c2cccc(-c3nc(-c4ccccc4)nc(-c4cccc(-n5c6ccccc6c6ccc(-c7cccc8c7sc7ccccc78)cc65)c4)n3)c2)cc1]

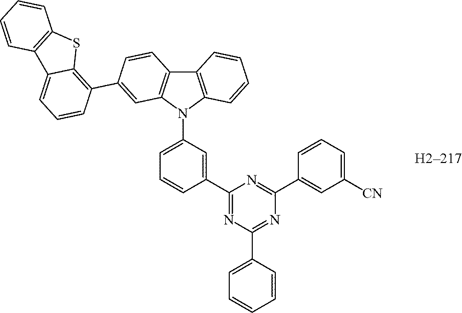 [SMILES: N#Cc1cccc(-c2nc(-c3ccccc3)nc(-c3cccc(-n4c5ccccc5c5ccc(-c6cccc7c6sc6ccccc67)cc54)c3)n2)c1]

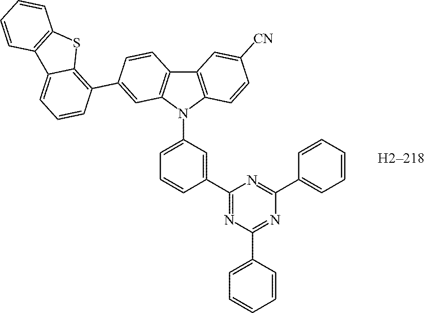 [SMILES: N#Cc1ccc2c(c1)c1ccc(-c3cccc4c3sc3ccccc34)cc1n2-c1cccc(-c2nc(-c3ccccc3)nc(-c3ccccc3)n2)c1]

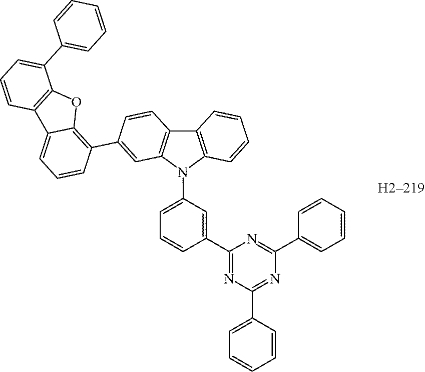 [SMILES: c1ccc(-c2nc(-c3ccccc3)nc(-c3cccc(-n4c5ccccc5c5ccc(-c6cccc7c6oc6c(-c8ccccc8)cccc67)cc54)c3)n2)cc1]

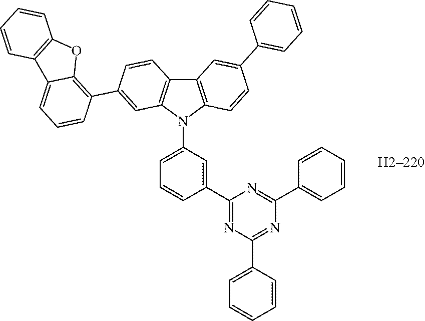 [SMILES: c1ccc(-c2ccc3c(c2)c2ccc(-c4cccc5c4oc4ccccc45)cc2n3-c2cccc(-c3nc(-c4ccccc4)nc(-c4ccccc4)n3)c2)cc1]

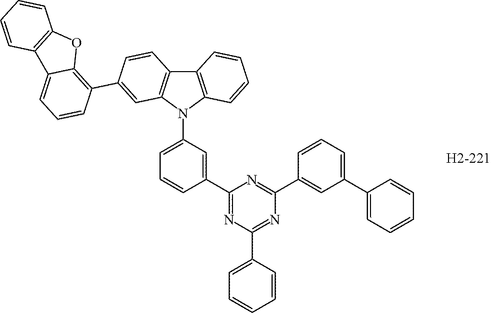 [SMILES: c1ccc(-c2cccc(-c3nc(-c4ccccc4)nc(-c4cccc(-n5c6ccccc6c6ccc(-c7cccc8c7oc7ccccc78)cc65)c4)n3)c2)cc1]

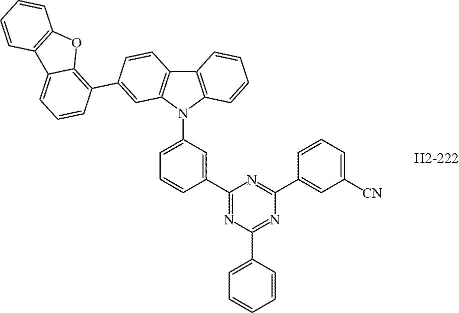 [SMILES: N#Cc1cccc(-c2nc(-c3ccccc3)nc(-c3cccc(-n4c5ccccc5c5ccc(-c6cccc7c6oc6ccccc67)cc54)c3)n2)c1]

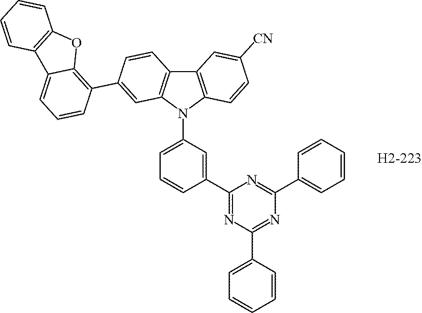 [SMILES: N#Cc1ccc2c(c1)c1ccc(-c3cccc4c3oc3ccccc34)cc1n2-c1cccc(-c2nc(-c3ccccc3)nc(-c3ccccc3)n2)c1]

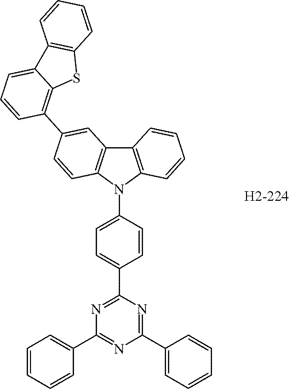 [SMILES: c1ccc(-c2nc(-c3ccccc3)nc(-c3ccc(-n4c5ccccc5c5cc(-c6cccc7c6sc6ccccc67)ccc54)cc3)n2)cc1]

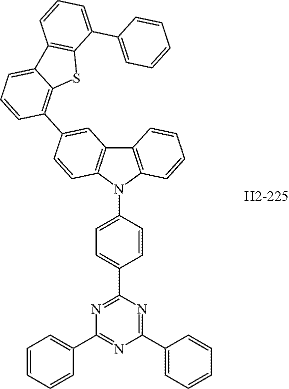 [SMILES: c1ccc(-c2nc(-c3ccccc3)nc(-c3ccc(-n4c5ccccc5c5cc(-c6cccc7c6sc6c(-c8ccccc8)cccc67)ccc54)cc3)n2)cc1]

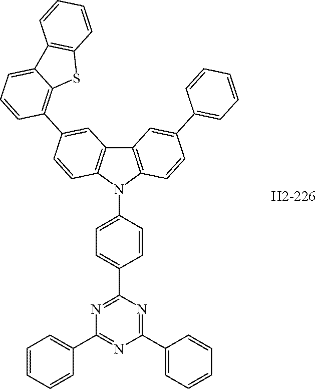 [SMILES: c1ccc(-c2ccc3c(c2)c2cc(-c4cccc5c4sc4ccccc45)ccc2n3-c2ccc(-c3nc(-c4ccccc4)nc(-c4ccccc4)n3)cc2)cc1]

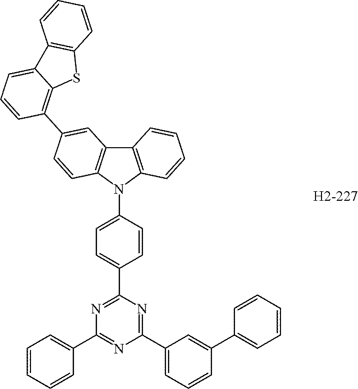 [SMILES: c1ccc(-c2cccc(-c3nc(-c4ccccc4)nc(-c4ccc(-n5c6ccccc6c6cc(-c7cccc8c7sc7ccccc78)ccc65)cc4)n3)c2)cc1]

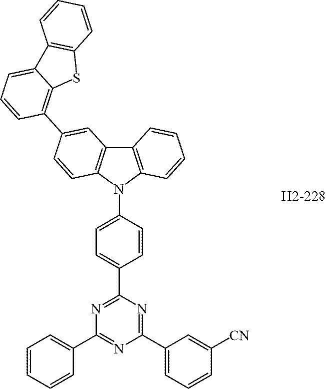 [SMILES: N#Cc1cccc(-c2nc(-c3ccccc3)nc(-c3ccc(-n4c5ccccc5c5cc(-c6cccc7c6sc6ccccc67)ccc54)cc3)n2)c1]

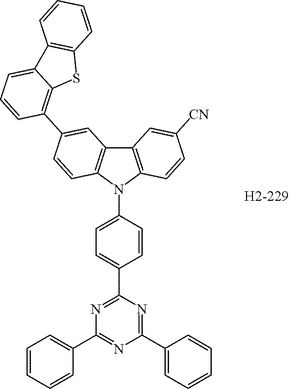 [SMILES: N#Cc1ccc2c(c1)c1cc(-c3cccc4c3sc3ccccc34)ccc1n2-c1ccc(-c2nc(-c3ccccc3)nc(-c3ccccc3)n2)cc1]

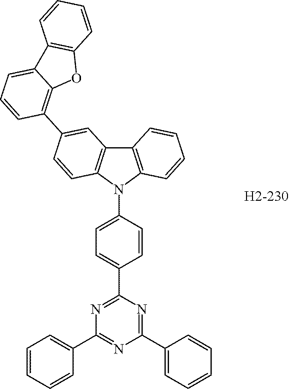 [SMILES: c1ccc(-c2nc(-c3ccccc3)nc(-c3ccc(-n4c5ccccc5c5cc(-c6cccc7c6oc6ccccc67)ccc54)cc3)n2)cc1]

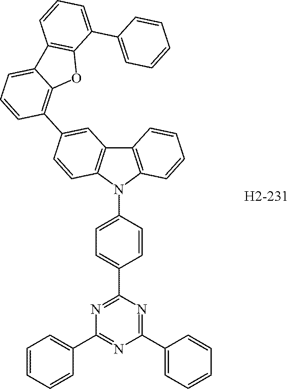 [SMILES: c1ccc(-c2nc(-c3ccccc3)nc(-c3ccc(-n4c5ccccc5c5cc(-c6cccc7c6oc6c(-c8ccccc8)cccc67)ccc54)cc3)n2)cc1]

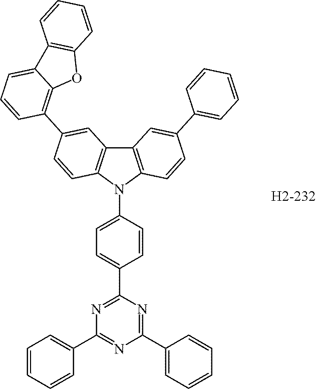 [SMILES: c1ccc(-c2ccc3c(c2)c2cc(-c4cccc5c4oc4ccccc45)ccc2n3-c2ccc(-c3nc(-c4ccccc4)nc(-c4ccccc4)n3)cc2)cc1]

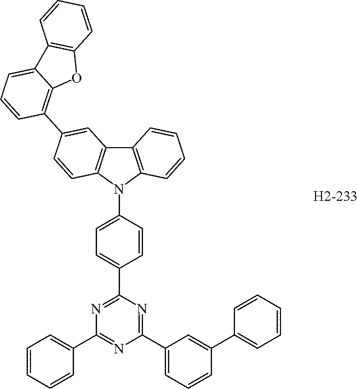 [SMILES: c1ccc(-c2cccc(-c3nc(-c4ccccc4)nc(-c4ccc(-n5c6ccccc6c6cc(-c7cccc8c7oc7ccccc78)ccc65)cc4)n3)c2)cc1]

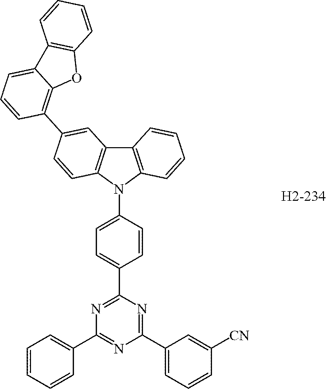 [SMILES: N#Cc1cccc(-c2nc(-c3ccccc3)nc(-c3ccc(-n4c5ccccc5c5cc(-c6cccc7c6oc6ccccc67)ccc54)cc3)n2)c1]

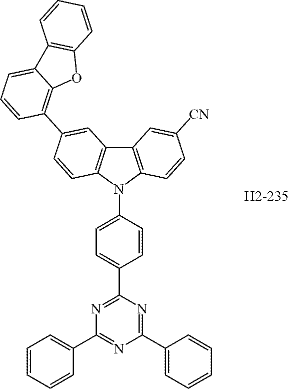 [SMILES: N#Cc1ccc2c(c1)c1cc(-c3cccc4c3oc3ccccc34)ccc1n2-c1ccc(-c2nc(-c3ccccc3)nc(-c3ccccc3)n2)cc1]

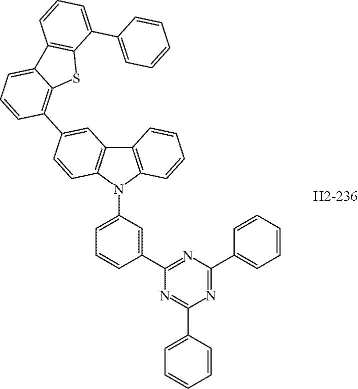 [SMILES: c1ccc(-c2nc(-c3ccccc3)nc(-c3cccc(-n4c5ccccc5c5cc(-c6cccc7c6sc6c(-c8ccccc8)cccc67)ccc54)c3)n2)cc1]

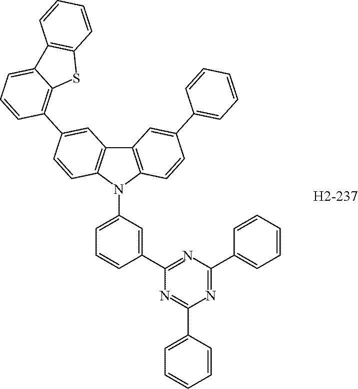 [SMILES: c1ccc(-c2ccc3c(c2)c2cc(-c4cccc5c4sc4ccccc45)ccc2n3-c2cccc(-c3nc(-c4ccccc4)nc(-c4ccccc4)n3)c2)cc1]

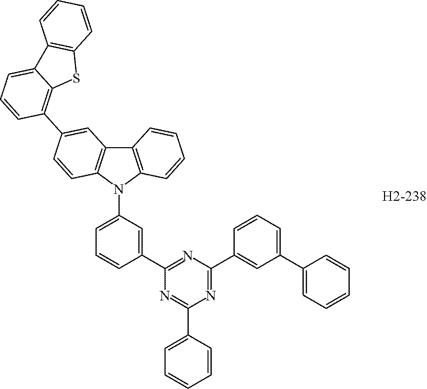 [SMILES: c1ccc(-c2cccc(-c3nc(-c4ccccc4)nc(-c4cccc(-n5c6ccccc6c6cc(-c7cccc8c7sc7ccccc78)ccc65)c4)n3)c2)cc1]

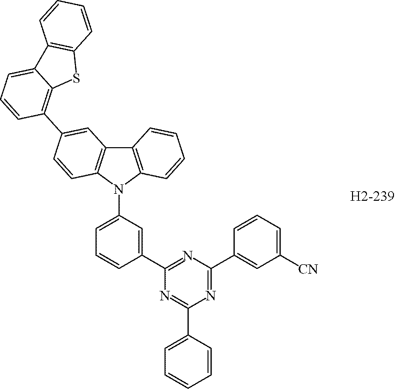 [SMILES: N#Cc1cccc(-c2nc(-c3ccccc3)nc(-c3cccc(-n4c5ccccc5c5cc(-c6cccc7c6sc6ccccc67)ccc54)c3)n2)c1]

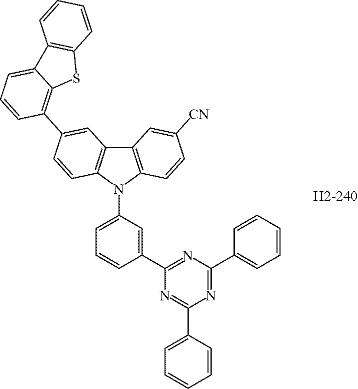 [SMILES: N#Cc1ccc2c(c1)c1cc(-c3cccc4c3sc3ccccc34)ccc1n2-c1cccc(-c2nc(-c3ccccc3)nc(-c3ccccc3)n2)c1]

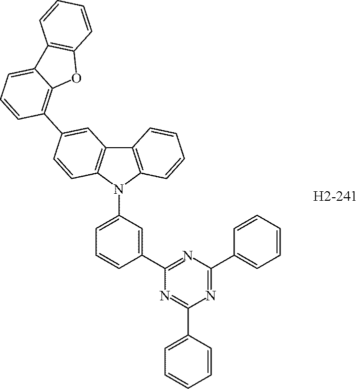 [SMILES: c1ccc(-c2nc(-c3ccccc3)nc(-c3cccc(-n4c5ccccc5c5cc(-c6cccc7c6oc6ccccc67)ccc54)c3)n2)cc1]

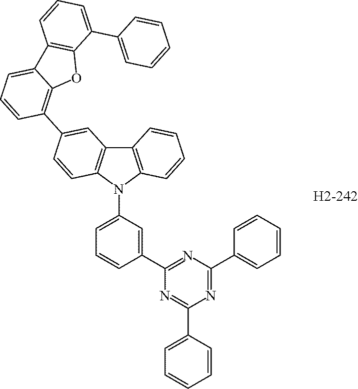 [SMILES: c1ccc(-c2nc(-c3ccccc3)nc(-c3cccc(-n4c5ccccc5c5cc(-c6cccc7c6oc6c(-c8ccccc8)cccc67)ccc54)c3)n2)cc1]

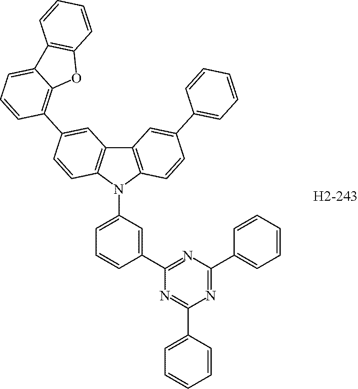 [SMILES: c1ccc(-c2ccc3c(c2)c2cc(-c4cccc5c4oc4ccccc45)ccc2n3-c2cccc(-c3nc(-c4ccccc4)nc(-c4ccccc4)n3)c2)cc1]

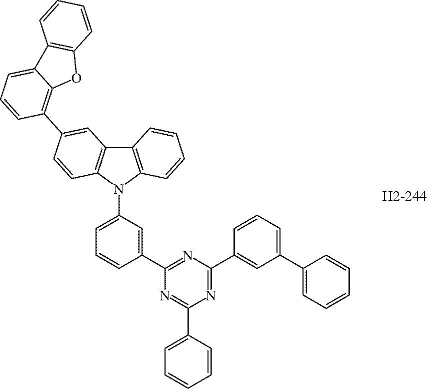 [SMILES: c1ccc(-c2cccc(-c3nc(-c4ccccc4)nc(-c4cccc(-n5c6ccccc6c6cc(-c7cccc8c7oc7ccccc78)ccc65)c4)n3)c2)cc1]

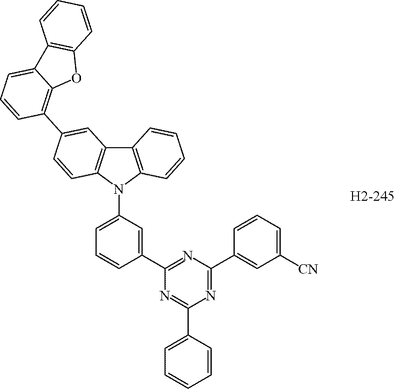 [SMILES: N#Cc1cccc(-c2nc(-c3ccccc3)nc(-c3cccc(-n4c5ccccc5c5cc(-c6cccc7c6oc6ccccc67)ccc54)c3)n2)c1]

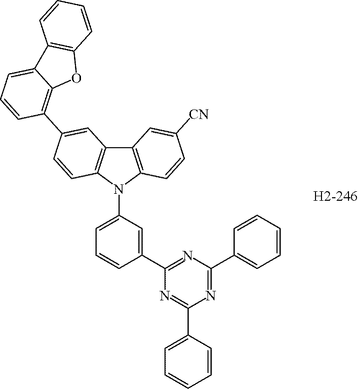 [SMILES: N#Cc1ccc2c(c1)c1cc(-c3cccc4c3oc3ccccc34)ccc1n2-c1cccc(-c2nc(-c3ccccc3)nc(-c3ccccc3)n2)c1]

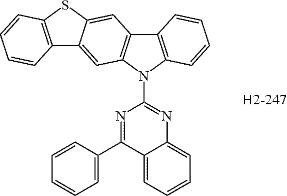 [SMILES: c1ccc(-c2nc(-n3c4ccccc4c4cc5sc6ccccc6c5cc43)nc3ccccc23)cc1]

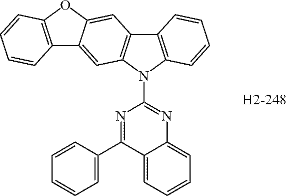 [SMILES: c1ccc(-c2nc(-n3c4ccccc4c4cc5oc6ccccc6c5cc43)nc3ccccc23)cc1]

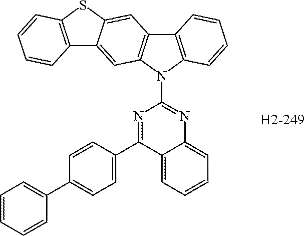 [SMILES: c1ccc(-c2ccc(-c3nc(-n4c5ccccc5c5cc6sc7ccccc7c6cc54)nc4ccccc34)cc2)cc1]

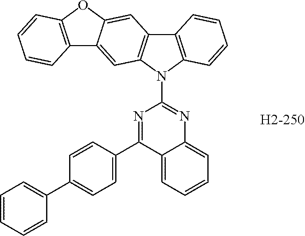 [SMILES: c1ccc(-c2ccc(-c3nc(-n4c5ccccc5c5cc6oc7ccccc7c6cc54)nc4ccccc34)cc2)cc1]

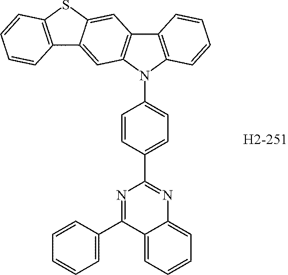 [SMILES: c1ccc(-c2nc(-c3ccc(-n4c5ccccc5c5cc6sc7ccccc7c6cc54)cc3)nc3ccccc23)cc1]

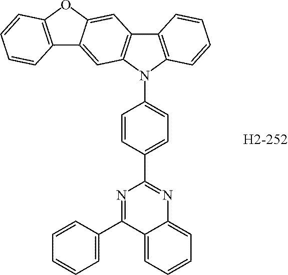 [SMILES: c1ccc(-c2nc(-c3ccc(-n4c5ccccc5c5cc6oc7ccccc7c6cc54)cc3)nc3ccccc23)cc1]

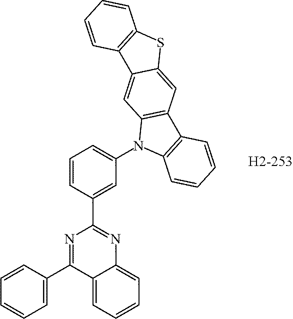 [SMILES: c1ccc(-c2nc(-c3cccc(-n4c5ccccc5c5cc6sc7ccccc7c6cc54)c3)nc3ccccc23)cc1]

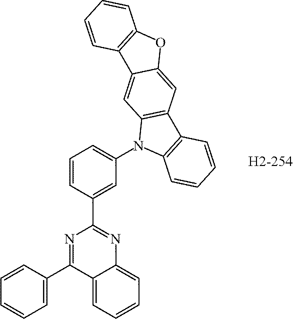 [SMILES: c1ccc(-c2nc(-c3cccc(-n4c5ccccc5c5cc6oc7ccccc7c6cc54)c3)nc3ccccc23)cc1]

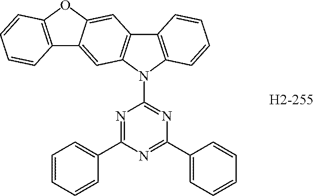 [SMILES: c1ccc(-c2nc(-c3ccccc3)nc(-n3c4ccccc4c4cc5oc6ccccc6c5cc43)n2)cc1]

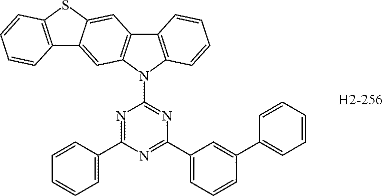 [SMILES: c1ccc(-c2cccc(-c3nc(-c4ccccc4)nc(-n4c5ccccc5c5cc6sc7ccccc7c6cc54)n3)c2)cc1]

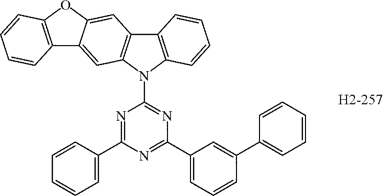 [SMILES: c1ccc(-c2cccc(-c3nc(-c4ccccc4)nc(-n4c5ccccc5c5cc6oc7ccccc7c6cc54)n3)c2)cc1]

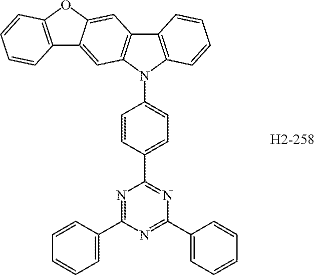 [SMILES: c1ccc(-c2nc(-c3ccccc3)nc(-c3ccc(-n4c5ccccc5c5cc6oc7ccccc7c6cc54)cc3)n2)cc1]

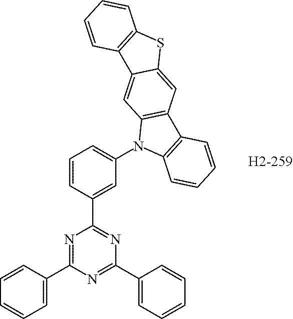 [SMILES: c1ccc(-c2nc(-c3ccccc3)nc(-c3cccc(-n4c5ccccc5c5cc6sc7ccccc7c6cc54)c3)n2)cc1]

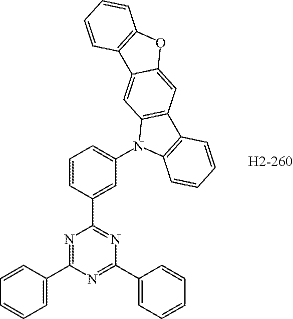 [SMILES: c1ccc(-c2nc(-c3ccccc3)nc(-c3cccc(-n4c5ccccc5c5cc6oc7ccccc7c6cc54)c3)n2)cc1]

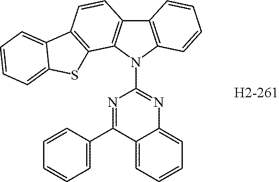 [SMILES: c1ccc(-c2nc(-n3c4ccccc4c4ccc5c6ccccc6sc5c43)nc3ccccc23)cc1]

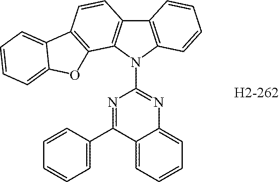 [SMILES: c1ccc(-c2nc(-n3c4ccccc4c4ccc5c6ccccc6oc5c43)nc3ccccc23)cc1]

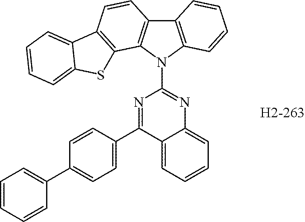 [SMILES: c1ccc(-c2ccc(-c3nc(-n4c5ccccc5c5ccc6c7ccccc7sc6c54)nc4ccccc34)cc2)cc1]

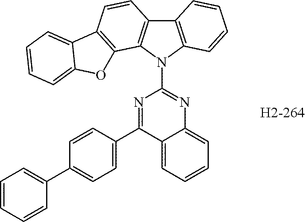 [SMILES: c1ccc(-c2ccc(-c3nc(-n4c5ccccc5c5ccc6c7ccccc7oc6c54)nc4ccccc34)cc2)cc1]

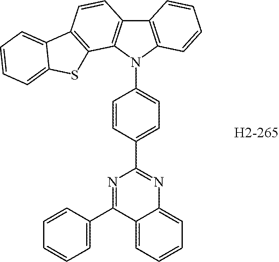 [SMILES: c1ccc(-c2nc(-c3ccc(-n4c5ccccc5c5ccc6c7ccccc7sc6c54)cc3)nc3ccccc23)cc1]

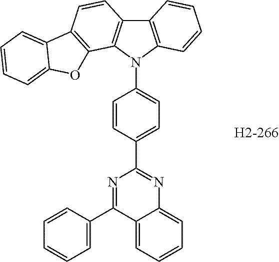 [SMILES: c1ccc(-c2nc(-c3ccc(-n4c5ccccc5c5ccc6c7ccccc7oc6c54)cc3)nc3ccccc23)cc1]

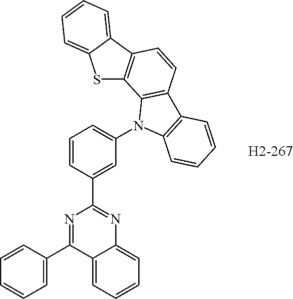 [SMILES: c1ccc(-c2nc(-c3cccc(-n4c5ccccc5c5ccc6c7ccccc7sc6c54)c3)nc3ccccc23)cc1]

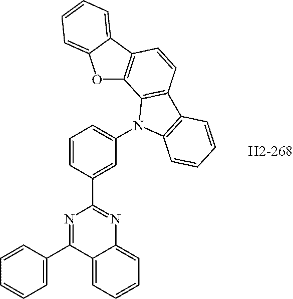 [SMILES: c1ccc(-c2nc(-c3cccc(-n4c5ccccc5c5ccc6c7ccccc7oc6c54)c3)nc3ccccc23)cc1]

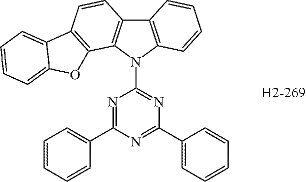 [SMILES: c1ccc(-c2nc(-c3ccccc3)nc(-n3c4ccccc4c4ccc5c6ccccc6oc5c43)n2)cc1]

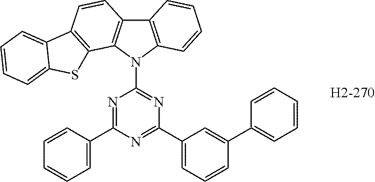 [SMILES: c1ccc(-c2cccc(-c3nc(-c4ccccc4)nc(-n4c5ccccc5c5ccc6c7ccccc7sc6c54)n3)c2)cc1]

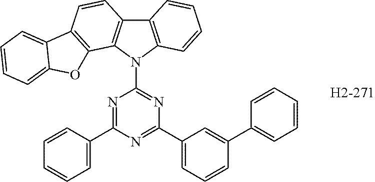 [SMILES: c1ccc(-c2cccc(-c3nc(-c4ccccc4)nc(-n4c5ccccc5c5ccc6c7ccccc7oc6c54)n3)c2)cc1]

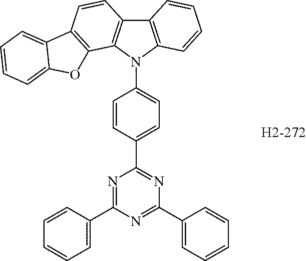 [SMILES: c1ccc(-c2nc(-c3ccccc3)nc(-c3ccc(-n4c5ccccc5c5ccc6c7ccccc7oc6c54)cc3)n2)cc1]

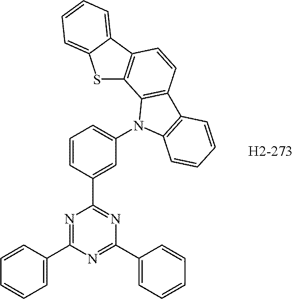 [SMILES: c1ccc(-c2nc(-c3ccccc3)nc(-c3cccc(-n4c5ccccc5c5ccc6c7ccccc7sc6c54)c3)n2)cc1]

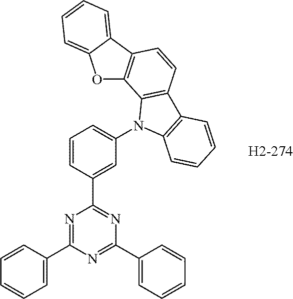 [SMILES: c1ccc(-c2nc(-c3ccccc3)nc(-c3cccc(-n4c5ccccc5c5ccc6c7ccccc7oc6c54)c3)n2)cc1]

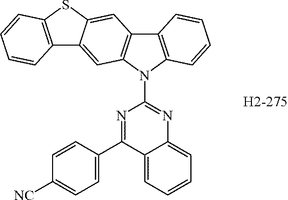

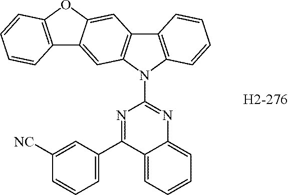

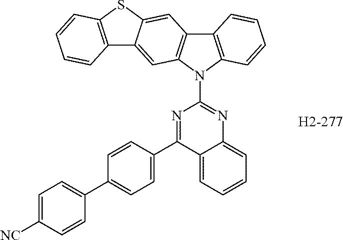

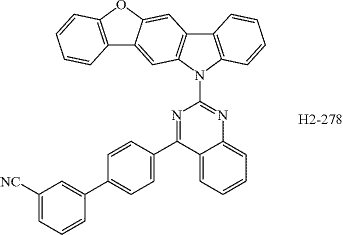

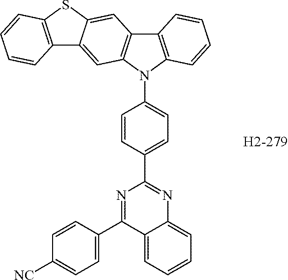

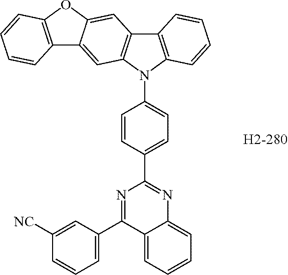

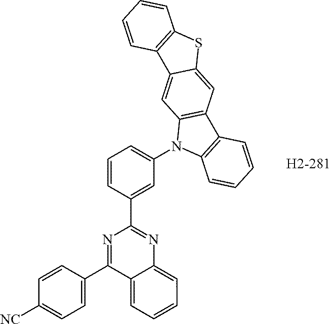

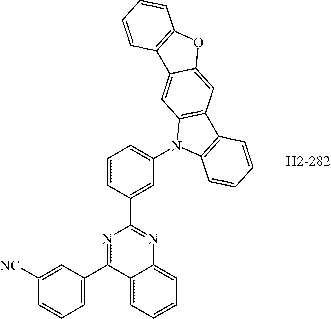

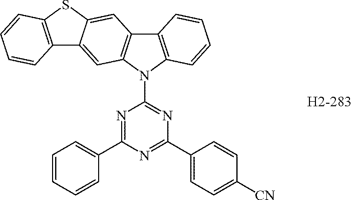 [SMILES: N#Cc1ccc(-c2nc(-c3ccccc3)nc(-n3c4ccccc4c4cc5sc6ccccc6c5cc43)n2)cc1]

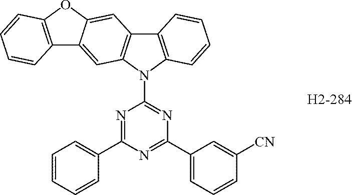 [SMILES: N#Cc1cccc(-c2nc(-c3ccccc3)nc(-n3c4ccccc4c4cc5oc6ccccc6c5cc43)n2)c1]

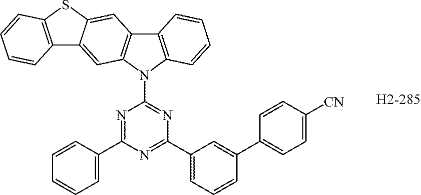 [SMILES: N#Cc1ccc(-c2cccc(-c3nc(-c4ccccc4)nc(-n4c5ccccc5c5cc6sc7ccccc7c6cc54)n3)c2)cc1]

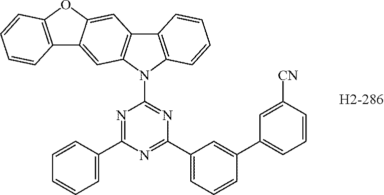 [SMILES: N#Cc1cccc(-c2cccc(-c3nc(-c4ccccc4)nc(-n4c5ccccc5c5cc6oc7ccccc7c6cc54)n3)c2)c1]

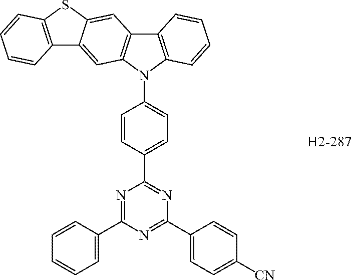 [SMILES: N#Cc1ccc(-c2nc(-c3ccccc3)nc(-c3ccc(-n4c5ccccc5c5cc6sc7ccccc7c6cc54)cc3)n2)cc1]

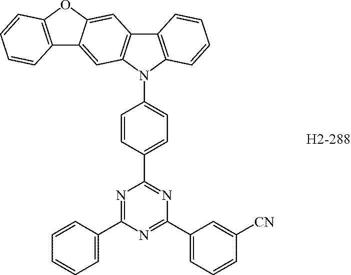 [SMILES: N#Cc1cccc(-c2nc(-c3ccccc3)nc(-c3ccc(-n4c5ccccc5c5cc6oc7ccccc7c6cc54)cc3)n2)c1]

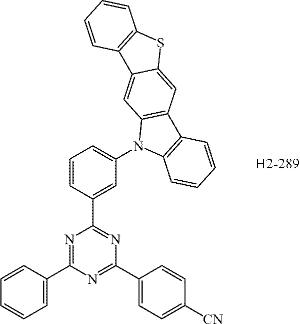 [SMILES: N#Cc1ccc(-c2nc(-c3ccccc3)nc(-c3cccc(-n4c5ccccc5c5cc6sc7ccccc7c6cc54)c3)n2)cc1]

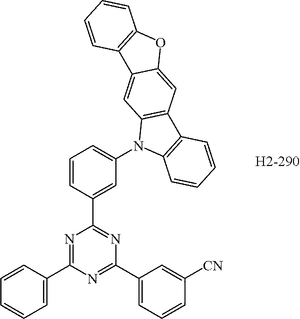 [SMILES: N#Cc1cccc(-c2nc(-c3ccccc3)nc(-c3cccc(-n4c5ccccc5c5cc6oc7ccccc7c6cc54)c3)n2)c1]

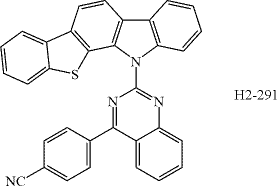

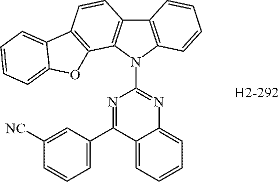

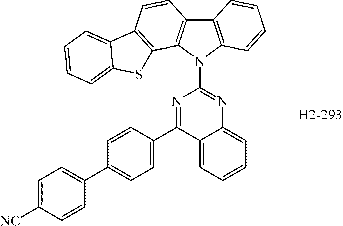

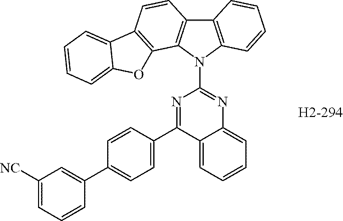

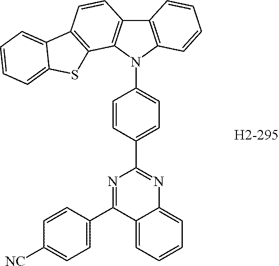

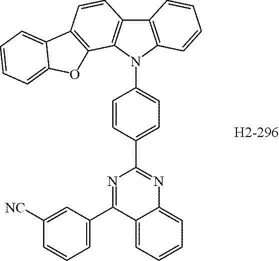

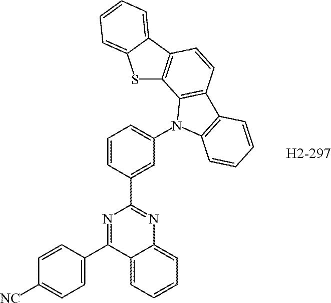

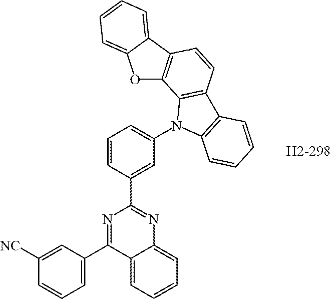

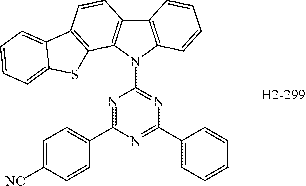

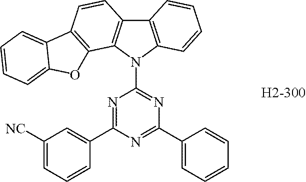

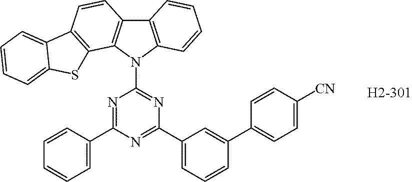 [SMILES: N#Cc1ccc(-c2cccc(-c3nc(-c4ccccc4)nc(-n4c5ccccc5c5ccc6c7ccccc7sc6c54)n3)c2)cc1]

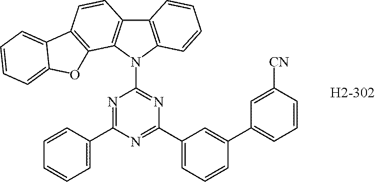 [SMILES: N#Cc1cccc(-c2cccc(-c3nc(-c4ccccc4)nc(-n4c5ccccc5c5ccc6c7ccccc7oc6c54)n3)c2)c1]

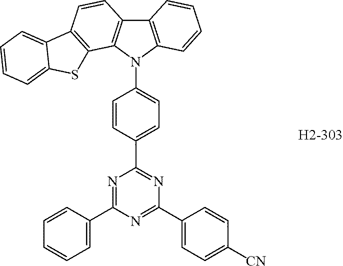 [SMILES: N#Cc1ccc(-c2nc(-c3ccccc3)nc(-c3ccc(-n4c5ccccc5c5ccc6c7ccccc7sc6c54)cc3)n2)cc1]

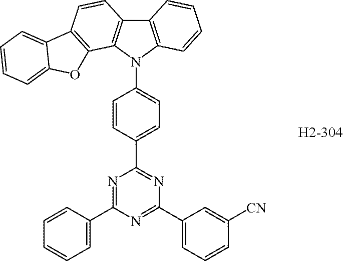 [SMILES: N#Cc1cccc(-c2nc(-c3ccccc3)nc(-c3ccc(-n4c5ccccc5c5ccc6c7ccccc7oc6c54)cc3)n2)c1]

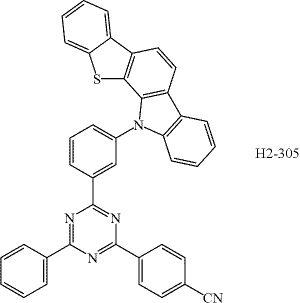 [SMILES: N#Cc1ccc(-c2nc(-c3ccccc3)nc(-c3cccc(-n4c5ccccc5c5ccc6c7ccccc7sc6c54)c3)n2)cc1]

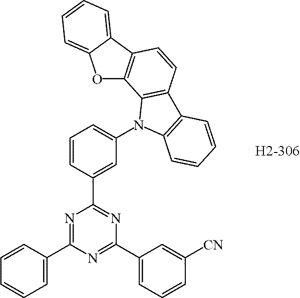 [SMILES: N#Cc1cccc(-c2nc(-c3ccccc3)nc(-c3cccc(-n4c5ccccc5c5ccc6c7ccccc7oc6c54)c3)n2)c1]

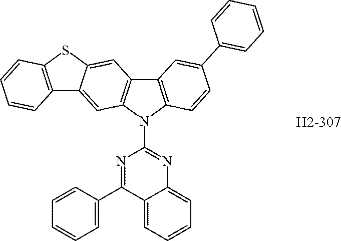 [SMILES: c1ccc(-c2ccc3c(c2)c2cc4sc5ccccc5c4cc2n3-c2nc(-c3ccccc3)c3ccccc3n2)cc1]

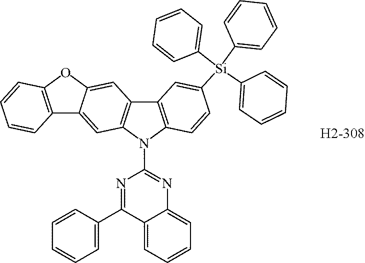 [SMILES: c1ccc(-c2nc(-n3c4ccc([Si](c5ccccc5)(c5ccccc5)c5ccccc5)cc4c4cc5oc6ccccc6c5cc43)nc3ccccc23)cc1]

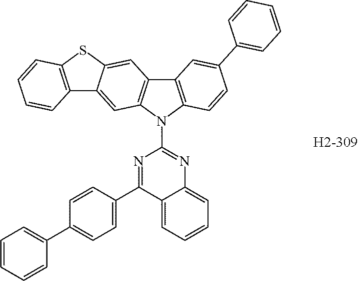 [SMILES: c1ccc(-c2ccc(-c3nc(-n4c5ccc(-c6ccccc6)cc5c5cc6sc7ccccc7c6cc54)nc4ccccc34)cc2)cc1]

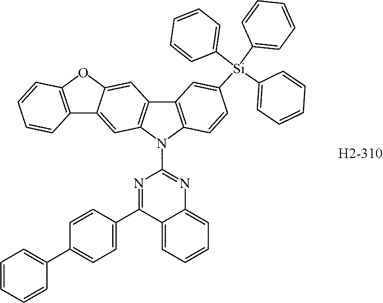 [SMILES: c1ccc(-c2ccc(-c3nc(-n4c5ccc([Si](c6ccccc6)(c6ccccc6)c6ccccc6)cc5c5cc6oc7ccccc7c6cc54)nc4ccccc34)cc2)cc1]

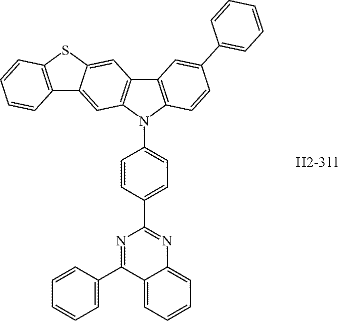 [SMILES: c1ccc(-c2ccc3c(c2)c2cc4sc5ccccc5c4cc2n3-c2ccc(-c3nc(-c4ccccc4)c4ccccc4n3)cc2)cc1]

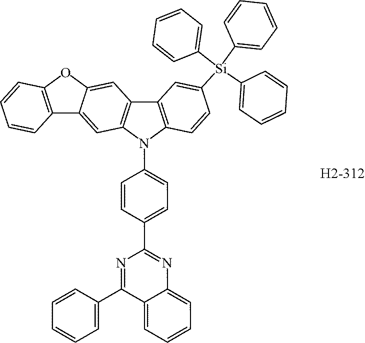 [SMILES: c1ccc(-c2nc(-c3ccc(-n4c5ccc([Si](c6ccccc6)(c6ccccc6)c6ccccc6)cc5c5cc6oc7ccccc7c6cc54)cc3)nc3ccccc23)cc1]

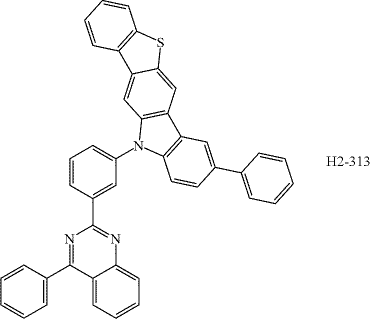 [SMILES: c1ccc(-c2ccc3c(c2)c2cc4sc5ccccc5c4cc2n3-c2cccc(-c3nc(-c4ccccc4)c4ccccc4n3)c2)cc1]

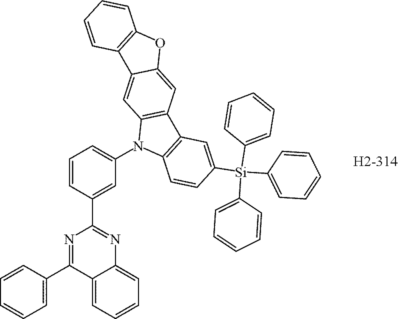 [SMILES: c1ccc(-c2nc(-c3cccc(-n4c5ccc([Si](c6ccccc6)(c6ccccc6)c6ccccc6)cc5c5cc6oc7ccccc7c6cc54)c3)nc3ccccc23)cc1]

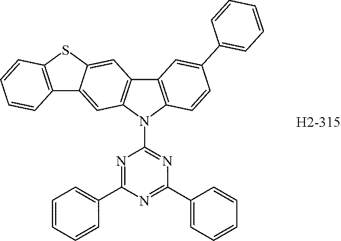 [SMILES: c1ccc(-c2ccc3c(c2)c2cc4sc5ccccc5c4cc2n3-c2nc(-c3ccccc3)nc(-c3ccccc3)n2)cc1]

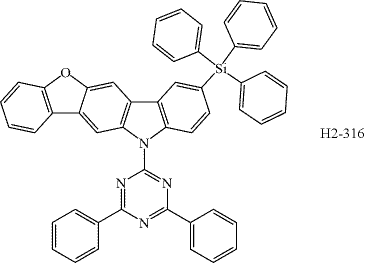 [SMILES: c1ccc(-c2nc(-c3ccccc3)nc(-n3c4ccc([Si](c5ccccc5)(c5ccccc5)c5ccccc5)cc4c4cc5oc6ccccc6c5cc43)n2)cc1]

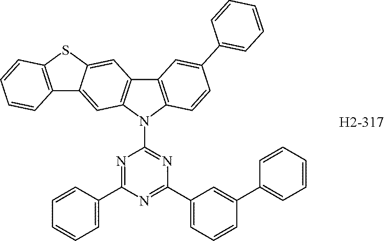 [SMILES: c1ccc(-c2cccc(-c3nc(-c4ccccc4)nc(-n4c5ccc(-c6ccccc6)cc5c5cc6sc7ccccc7c6cc54)n3)c2)cc1]

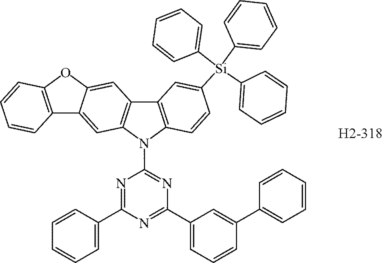 [SMILES: c1ccc(-c2cccc(-c3nc(-c4ccccc4)nc(-n4c5ccc([Si](c6ccccc6)(c6ccccc6)c6ccccc6)cc5c5cc6oc7ccccc7c6cc54)n3)c2)cc1]

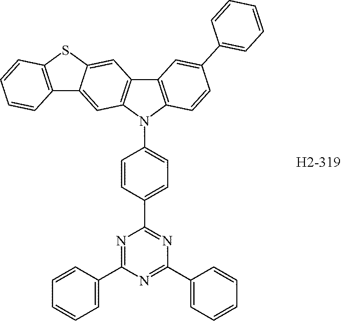 [SMILES: c1ccc(-c2ccc3c(c2)c2cc4sc5ccccc5c4cc2n3-c2ccc(-c3nc(-c4ccccc4)nc(-c4ccccc4)n3)cc2)cc1]

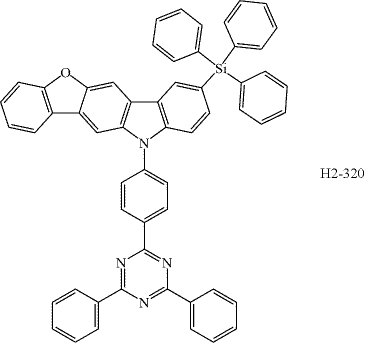 [SMILES: c1ccc(-c2nc(-c3ccccc3)nc(-c3ccc(-n4c5ccc([Si](c6ccccc6)(c6ccccc6)c6ccccc6)cc5c5cc6oc7ccccc7c6cc54)cc3)n2)cc1]

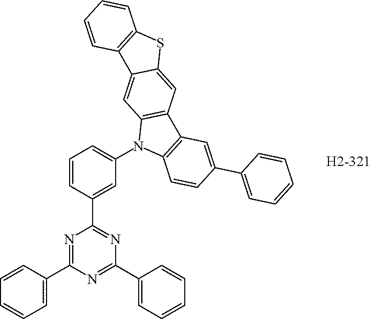 [SMILES: c1ccc(-c2ccc3c(c2)c2cc4sc5ccccc5c4cc2n3-c2cccc(-c3nc(-c4ccccc4)nc(-c4ccccc4)n3)c2)cc1]

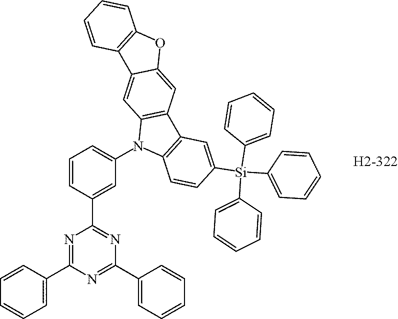 [SMILES: c1ccc(-c2nc(-c3ccccc3)nc(-c3cccc(-n4c5ccc([Si](c6ccccc6)(c6ccccc6)c6ccccc6)cc5c5cc6oc7ccccc7c6cc54)c3)n2)cc1]

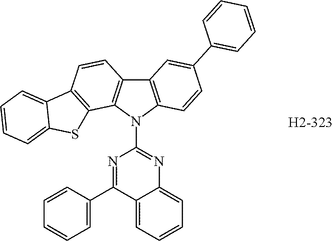 [SMILES: c1ccc(-c2ccc3c(c2)c2ccc4c5ccccc5sc4c2n3-c2nc(-c3ccccc3)c3ccccc3n2)cc1]

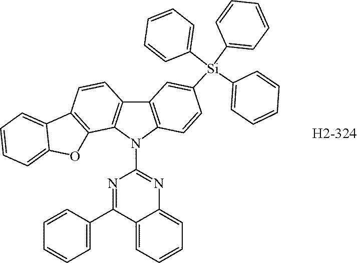 [SMILES: c1ccc(-c2nc(-n3c4ccc([Si](c5ccccc5)(c5ccccc5)c5ccccc5)cc4c4ccc5c6ccccc6oc5c43)nc3ccccc23)cc1]

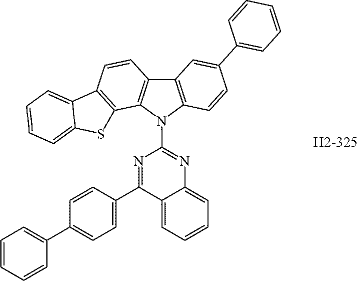 [SMILES: c1ccc(-c2ccc(-c3nc(-n4c5ccc(-c6ccccc6)cc5c5ccc6c7ccccc7sc6c54)nc4ccccc34)cc2)cc1]

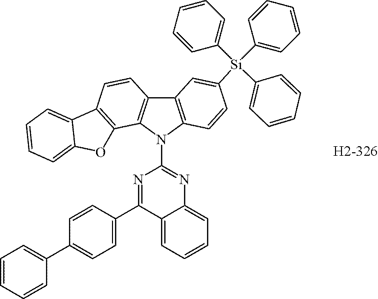 [SMILES: c1ccc(-c2ccc(-c3nc(-n4c5ccc([Si](c6ccccc6)(c6ccccc6)c6ccccc6)cc5c5ccc6c7ccccc7oc6c54)nc4ccccc34)cc2)cc1]

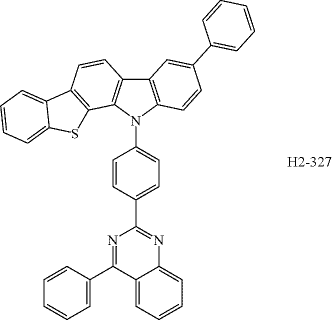 [SMILES: c1ccc(-c2ccc3c(c2)c2ccc4c5ccccc5sc4c2n3-c2ccc(-c3nc(-c4ccccc4)c4ccccc4n3)cc2)cc1]

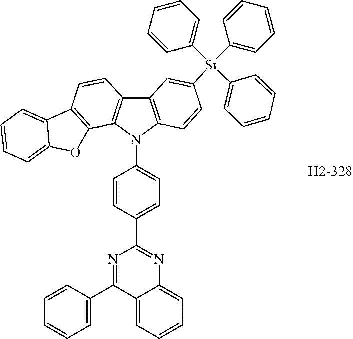 [SMILES: c1ccc(-c2nc(-c3ccc(-n4c5ccc([Si](c6ccccc6)(c6ccccc6)c6ccccc6)cc5c5ccc6c7ccccc7oc6c54)cc3)nc3ccccc23)cc1]

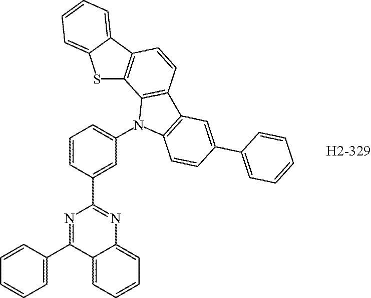 [SMILES: c1ccc(-c2ccc3c(c2)c2ccc4c5ccccc5sc4c2n3-c2cccc(-c3nc(-c4ccccc4)c4ccccc4n3)c2)cc1]

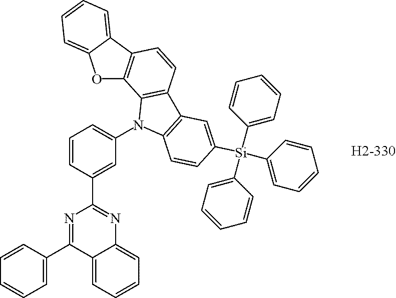 [SMILES: c1ccc(-c2nc(-c3cccc(-n4c5ccc([Si](c6ccccc6)(c6ccccc6)c6ccccc6)cc5c5ccc6c7ccccc7oc6c54)c3)nc3ccccc23)cc1]

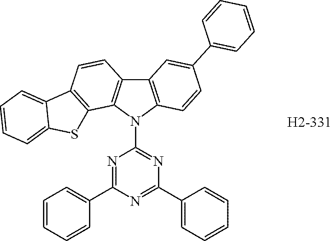 [SMILES: c1ccc(-c2ccc3c(c2)c2ccc4c5ccccc5sc4c2n3-c2nc(-c3ccccc3)nc(-c3ccccc3)n2)cc1]

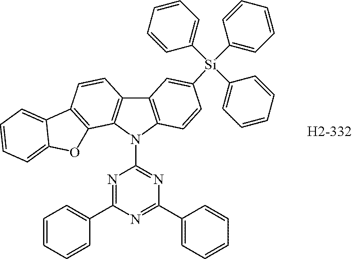 [SMILES: c1ccc(-c2nc(-c3ccccc3)nc(-n3c4ccc([Si](c5ccccc5)(c5ccccc5)c5ccccc5)cc4c4ccc5c6ccccc6oc5c43)n2)cc1]

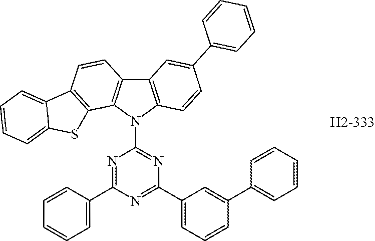 [SMILES: c1ccc(-c2cccc(-c3nc(-c4ccccc4)nc(-n4c5ccc(-c6ccccc6)cc5c5ccc6c7ccccc7sc6c54)n3)c2)cc1]

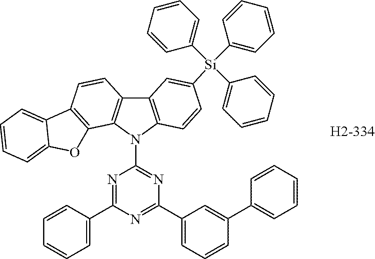 [SMILES: c1ccc(-c2cccc(-c3nc(-c4ccccc4)nc(-n4c5ccc([Si](c6ccccc6)(c6ccccc6)c6ccccc6)cc5c5ccc6c7ccccc7oc6c54)n3)c2)cc1]

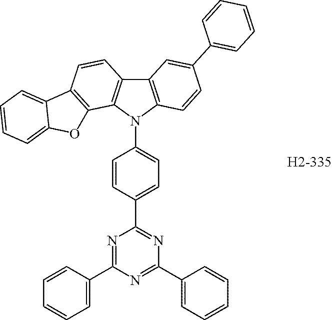 [SMILES: c1ccc(-c2ccc3c(c2)c2ccc4c5ccccc5oc4c2n3-c2ccc(-c3nc(-c4ccccc4)nc(-c4ccccc4)n3)cc2)cc1]

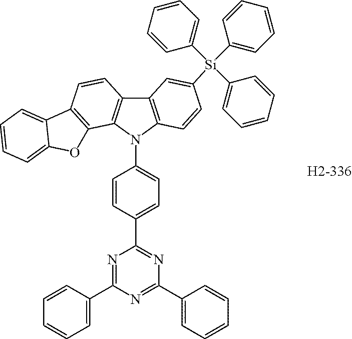 [SMILES: c1ccc(-c2nc(-c3ccccc3)nc(-c3ccc(-n4c5ccc([Si](c6ccccc6)(c6ccccc6)c6ccccc6)cc5c5ccc6c7ccccc7oc6c54)cc3)n2)cc1]

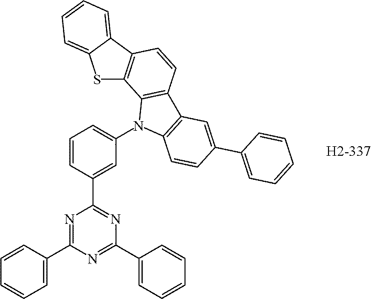 [SMILES: c1ccc(-c2ccc3c(c2)c2ccc4c5ccccc5sc4c2n3-c2cccc(-c3nc(-c4ccccc4)nc(-c4ccccc4)n3)c2)cc1]

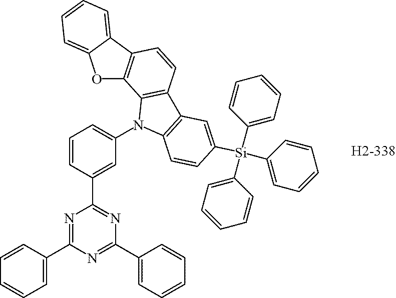 [SMILES: c1ccc(-c2nc(-c3ccccc3)nc(-c3cccc(-n4c5ccc([Si](c6ccccc6)(c6ccccc6)c6ccccc6)cc5c5ccc6c7ccccc7oc6c54)c3)n2)cc1]

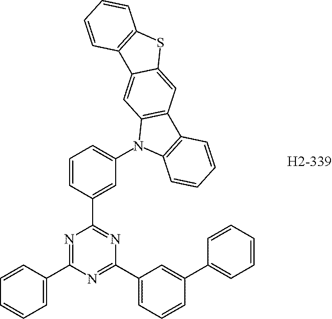 [SMILES: c1ccc(-c2cccc(-c3nc(-c4ccccc4)nc(-c4cccc(-n5c6ccccc6c6cc7sc8ccccc8c7cc65)c4)n3)c2)cc1]

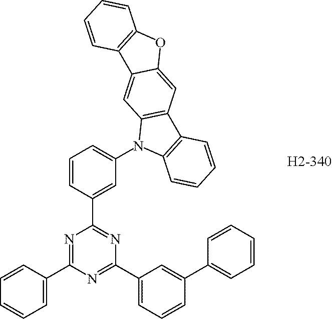 [SMILES: c1ccc(-c2cccc(-c3nc(-c4ccccc4)nc(-c4cccc(-n5c6ccccc6c6cc7oc8ccccc8c7cc65)c4)n3)c2)cc1]

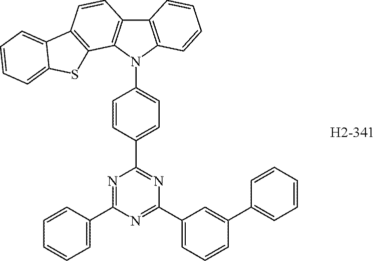 [SMILES: c1ccc(-c2cccc(-c3nc(-c4ccccc4)nc(-c4ccc(-n5c6ccccc6c6ccc7c8ccccc8sc7c65)cc4)n3)c2)cc1]

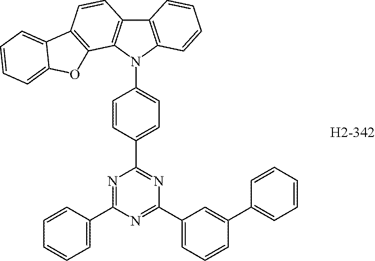 [SMILES: c1ccc(-c2cccc(-c3nc(-c4ccccc4)nc(-c4ccc(-n5c6ccccc6c6ccc7c8ccccc8oc7c65)cc4)n3)c2)cc1]

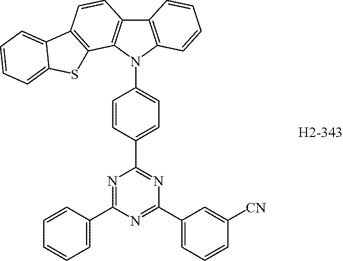 [SMILES: N#Cc1cccc(-c2nc(-c3ccccc3)nc(-c3ccc(-n4c5ccccc5c5ccc6c7ccccc7sc6c54)cc3)n2)c1]

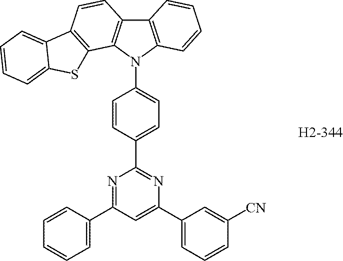 [SMILES: N#Cc1cccc(-c2cc(-c3ccccc3)nc(-c3ccc(-n4c5ccccc5c5ccc6c7ccccc7sc6c54)cc3)n2)c1]

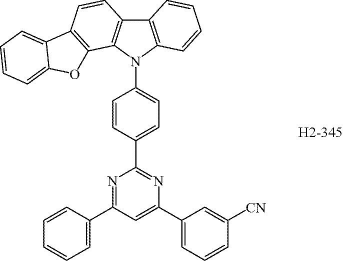 [SMILES: N#Cc1cccc(-c2cc(-c3ccccc3)nc(-c3ccc(-n4c5ccccc5c5ccc6c7ccccc7oc6c54)cc3)n2)c1]

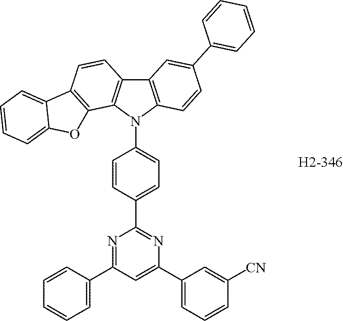 [SMILES: N#Cc1cccc(-c2cc(-c3ccccc3)nc(-c3ccc(-n4c5ccc(-c6ccccc6)cc5c5ccc6c7ccccc7oc6c54)cc3)n2)c1]

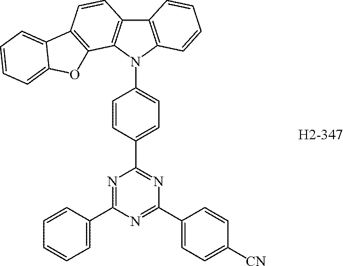 [SMILES: N#Cc1ccc(-c2nc(-c3ccccc3)nc(-c3ccc(-n4c5ccccc5c5ccc6c7ccccc7oc6c54)cc3)n2)cc1]

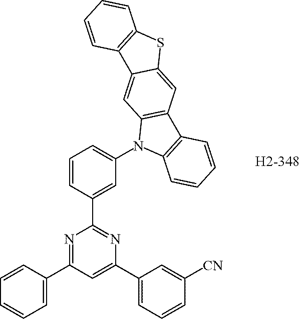 [SMILES: N#Cc1cccc(-c2cc(-c3ccccc3)nc(-c3cccc(-n4c5ccccc5c5cc6sc7ccccc7c6cc54)c3)n2)c1]

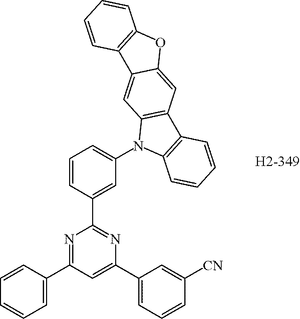 [SMILES: N#Cc1cccc(-c2cc(-c3ccccc3)nc(-c3cccc(-n4c5ccccc5c5cc6oc7ccccc7c6cc54)c3)n2)c1]

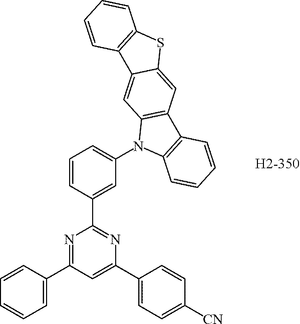 [SMILES: N#Cc1ccc(-c2cc(-c3ccccc3)nc(-c3cccc(-n4c5ccccc5c5cc6sc7ccccc7c6cc54)c3)n2)cc1]

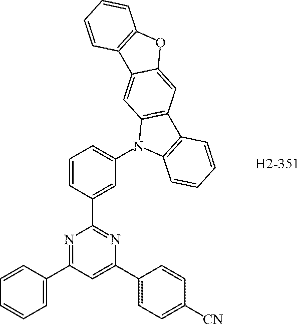 [SMILES: N#Cc1ccc(-c2cc(-c3ccccc3)nc(-c3cccc(-n4c5ccccc5c5cc6oc7ccccc7c6cc54)c3)n2)cc1]

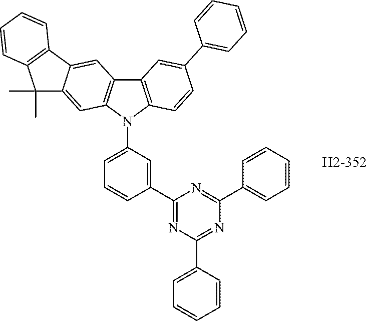 [SMILES: CC1(C)c2ccccc2-c2cc3c4cc(-c5ccccc5)ccc4n(-c4cccc(-c5nc(-c6ccccc6)nc(-c6ccccc6)n5)c4)c3cc21]

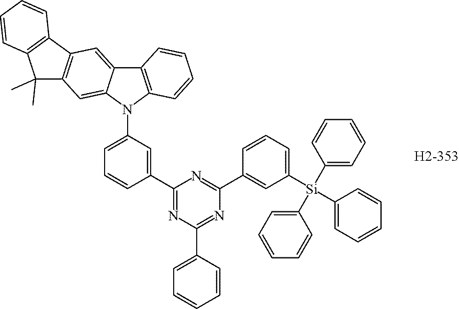 [SMILES: CC1(C)c2ccccc2-c2cc3c4ccccc4n(-c4cccc(-c5nc(-c6ccccc6)nc(-c6cccc([Si](c7ccccc7)(c7ccccc7)c7ccccc7)c6)n5)c4)c3cc21]

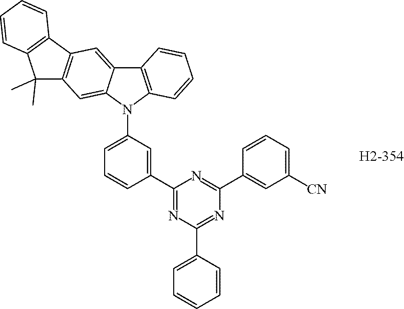 [SMILES: CC1(C)c2ccccc2-c2cc3c4ccccc4n(-c4cccc(-c5nc(-c6ccccc6)nc(-c6cccc(C#N)c6)n5)c4)c3cc21]

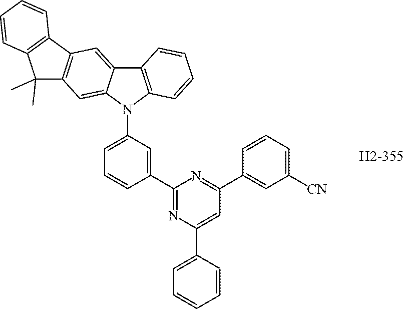 [SMILES: CC1(C)c2ccccc2-c2cc3c4ccccc4n(-c4cccc(-c5nc(-c6ccccc6)cc(-c6cccc(C#N)c6)n5)c4)c3cc21]

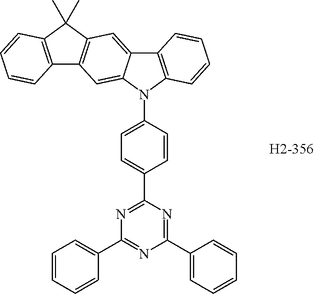 [SMILES: CC1(C)c2ccccc2-c2cc3c(cc21)c1ccccc1n3-c1ccc(-c2nc(-c3ccccc3)nc(-c3ccccc3)n2)cc1]

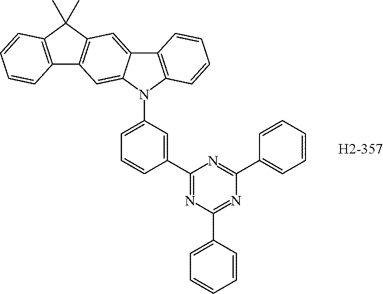 [SMILES: CC1(C)c2ccccc2-c2cc3c(cc21)c1ccccc1n3-c1cccc(-c2nc(-c3ccccc3)nc(-c3ccccc3)n2)c1]

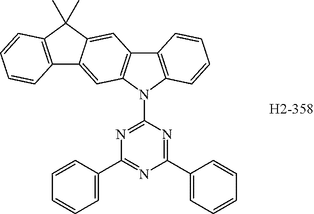 [SMILES: CC1(C)c2ccccc2-c2cc3c(cc21)c1ccccc1n3-c1nc(-c2ccccc2)nc(-c2ccccc2)n1]

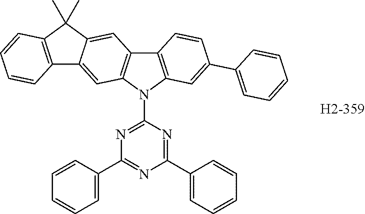 [SMILES: CC1(C)c2ccccc2-c2cc3c(cc21)c1ccc(-c2ccccc2)cc1n3-c1nc(-c2ccccc2)nc(-c2ccccc2)n1]

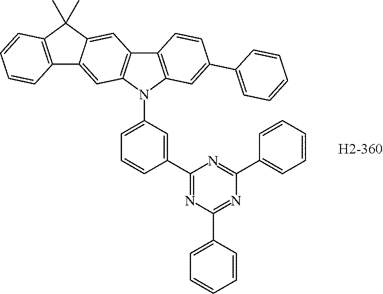 [SMILES: CC1(C)c2ccccc2-c2cc3c(cc21)c1ccc(-c2ccccc2)cc1n3-c1cccc(-c2nc(-c3ccccc3)nc(-c3ccccc3)n2)c1]

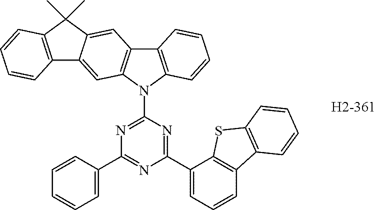 [SMILES: CC1(C)c2ccccc2-c2cc3c(cc21)c1ccccc1n3-c1nc(-c2ccccc2)nc(-c2cccc3c2sc2ccccc23)n1]

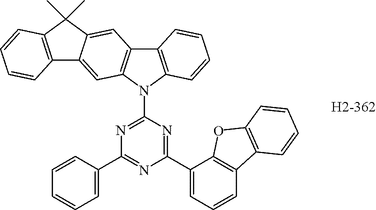 [SMILES: CC1(C)c2ccccc2-c2cc3c(cc21)c1ccccc1n3-c1nc(-c2ccccc2)nc(-c2cccc3c2oc2ccccc23)n1]

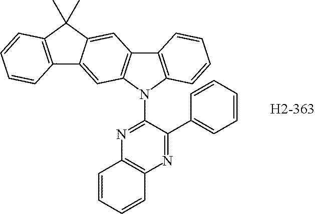 [SMILES: CC1(C)c2ccccc2-c2cc3c(cc21)c1ccccc1n3-c1nc2ccccc2nc1-c1ccccc1]

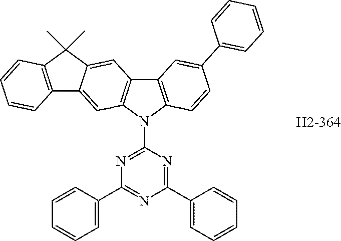 [SMILES: CC1(C)c2ccccc2-c2cc3c(cc21)c1cc(-c2ccccc2)ccc1n3-c1nc(-c2ccccc2)nc(-c2ccccc2)n1]

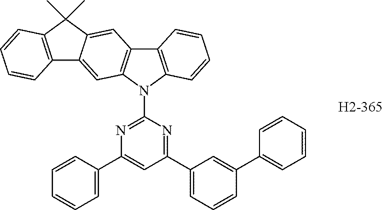 [SMILES: CC1(C)c2ccccc2-c2cc3c(cc21)c1ccccc1n3-c1nc(-c2ccccc2)cc(-c2cccc(-c3ccccc3)c2)n1]

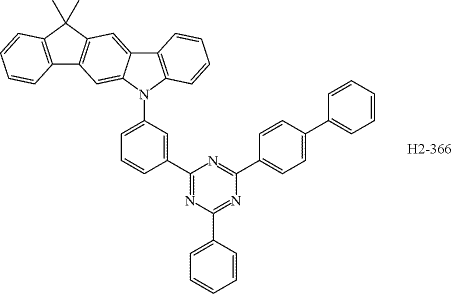 [SMILES: CC1(C)c2ccccc2-c2cc3c(cc21)c1ccccc1n3-c1cccc(-c2nc(-c3ccccc3)nc(-c3ccc(-c4ccccc4)cc3)n2)c1]

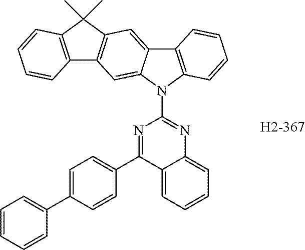 [SMILES: CC1(C)c2ccccc2-c2cc3c(cc21)c1ccccc1n3-c1nc(-c2ccc(-c3ccccc3)cc2)c2ccccc2n1]

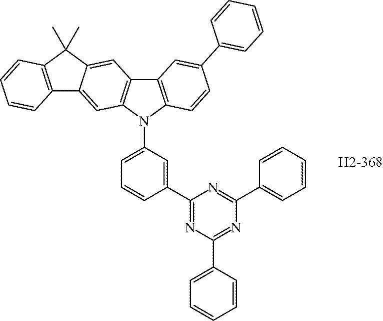 [SMILES: CC1(C)c2ccccc2-c2cc3c(cc21)c1cc(-c2ccccc2)ccc1n3-c1cccc(-c2nc(-c3ccccc3)nc(-c3ccccc3)n2)c1]

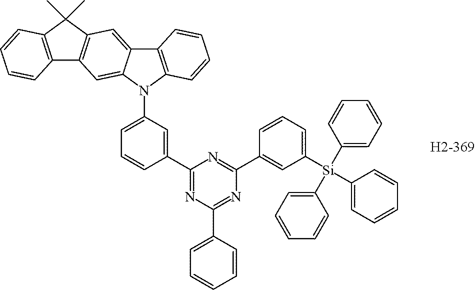 [SMILES: CC1(C)c2ccccc2-c2cc3c(cc21)c1ccccc1n3-c1cccc(-c2nc(-c3ccccc3)nc(-c3cccc([Si](c4ccccc4)(c4ccccc4)c4ccccc4)c3)n2)c1]

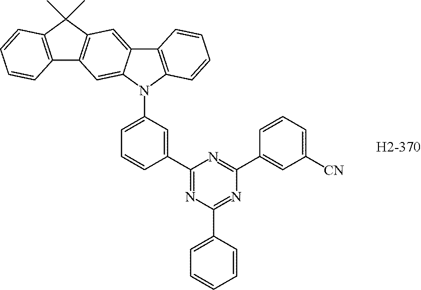 [SMILES: CC1(C)c2ccccc2-c2cc3c(cc21)c1ccccc1n3-c1cccc(-c2nc(-c3ccccc3)nc(-c3cccc(C#N)c3)n2)c1]

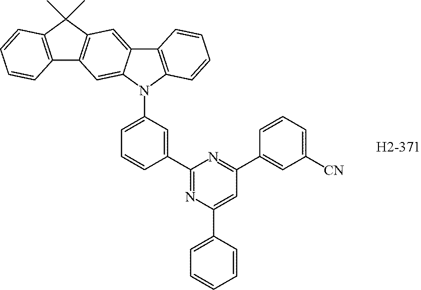 [SMILES: CC1(C)c2ccccc2-c2cc3c(cc21)c1ccccc1n3-c1cccc(-c2nc(-c3ccccc3)cc(-c3cccc(C#N)c3)n2)c1]

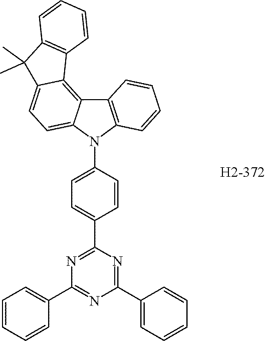 [SMILES: CC1(C)c2ccccc2-c2c1ccc1c2c2ccccc2n1-c1ccc(-c2nc(-c3ccccc3)nc(-c3ccccc3)n2)cc1]

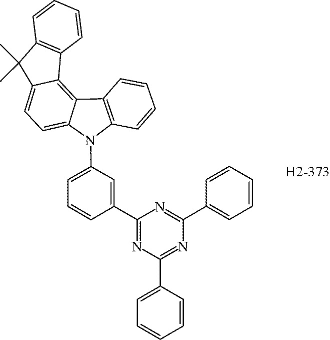 [SMILES: CC1(C)c2ccccc2-c2c1ccc1c2c2ccccc2n1-c1cccc(-c2nc(-c3ccccc3)nc(-c3ccccc3)n2)c1]

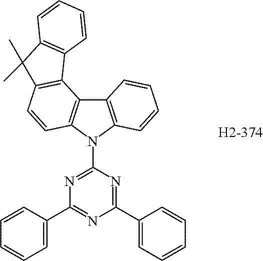 [SMILES: CC1(C)c2ccccc2-c2c1ccc1c2c2ccccc2n1-c1nc(-c2ccccc2)nc(-c2ccccc2)n1]

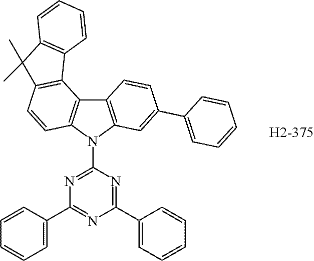 [SMILES: CC1(C)c2ccccc2-c2c1ccc1c2c2ccc(-c3ccccc3)cc2n1-c1nc(-c2ccccc2)nc(-c2ccccc2)n1]

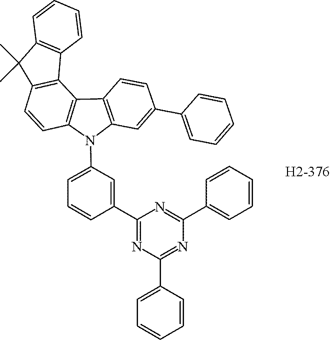 [SMILES: CC1(C)c2ccccc2-c2c1ccc1c2c2ccc(-c3ccccc3)cc2n1-c1cccc(-c2nc(-c3ccccc3)nc(-c3ccccc3)n2)c1]

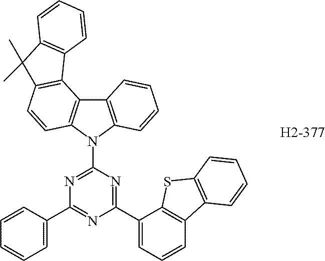 [SMILES: CC1(C)c2ccccc2-c2c1ccc1c2c2ccccc2n1-c1nc(-c2ccccc2)nc(-c2cccc3c2sc2ccccc23)n1]

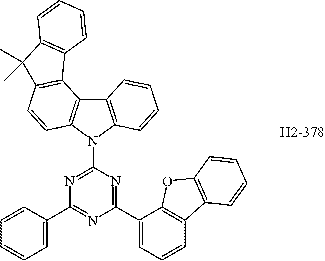 [SMILES: CC1(C)c2ccccc2-c2c1ccc1c2c2ccccc2n1-c1nc(-c2ccccc2)nc(-c2cccc3c2oc2ccccc23)n1]

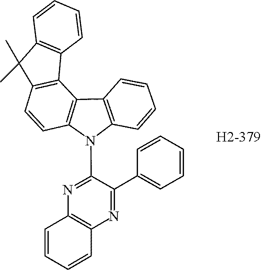 [SMILES: CC1(C)c2ccccc2-c2c1ccc1c2c2ccccc2n1-c1nc2ccccc2nc1-c1ccccc1]

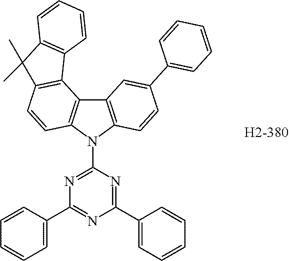 [SMILES: CC1(C)c2ccccc2-c2c1ccc1c2c2cc(-c3ccccc3)ccc2n1-c1nc(-c2ccccc2)nc(-c2ccccc2)n1]

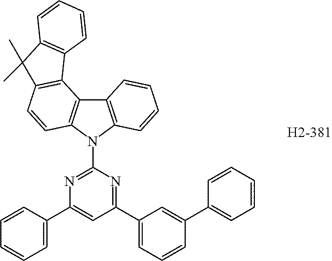 [SMILES: CC1(C)c2ccccc2-c2c1ccc1c2c2ccccc2n1-c1nc(-c2ccccc2)cc(-c2cccc(-c3ccccc3)c2)n1]

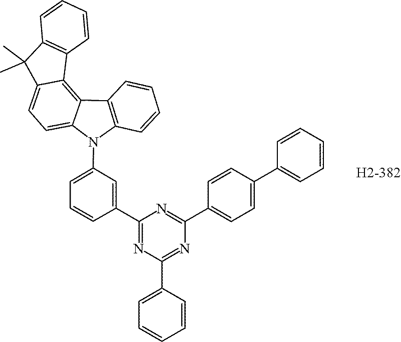 [SMILES: CC1(C)c2ccccc2-c2c1ccc1c2c2ccccc2n1-c1cccc(-c2nc(-c3ccccc3)nc(-c3ccc(-c4ccccc4)cc3)n2)c1]

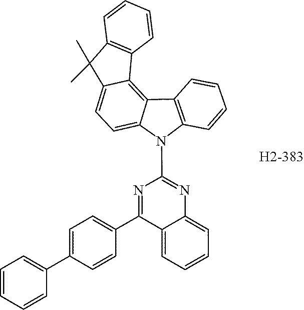 [SMILES: CC1(C)c2ccccc2-c2c1ccc1c2c2ccccc2n1-c1nc(-c2ccc(-c3ccccc3)cc2)c2ccccc2n1]

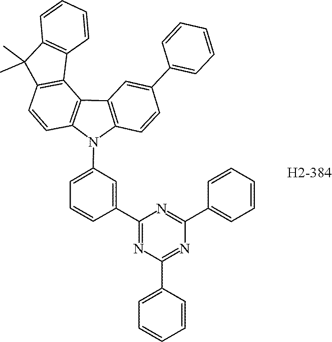 [SMILES: CC1(C)c2ccccc2-c2c1ccc1c2c2cc(-c3ccccc3)ccc2n1-c1cccc(-c2nc(-c3ccccc3)nc(-c3ccccc3)n2)c1]

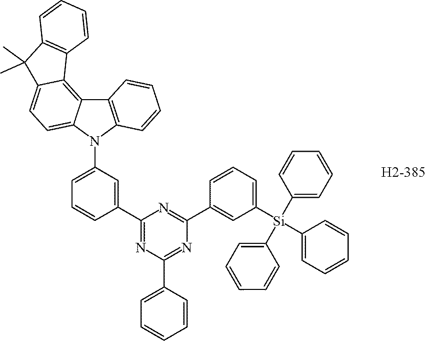 [SMILES: CC1(C)c2ccccc2-c2c1ccc1c2c2ccccc2n1-c1cccc(-c2nc(-c3ccccc3)nc(-c3cccc([Si](c4ccccc4)(c4ccccc4)c4ccccc4)c3)n2)c1]

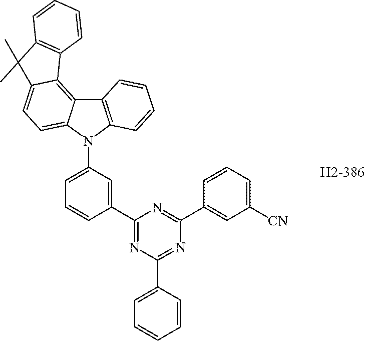 [SMILES: CC1(C)c2ccccc2-c2c1ccc1c2c2ccccc2n1-c1cccc(-c2nc(-c3ccccc3)nc(-c3cccc(C#N)c3)n2)c1]

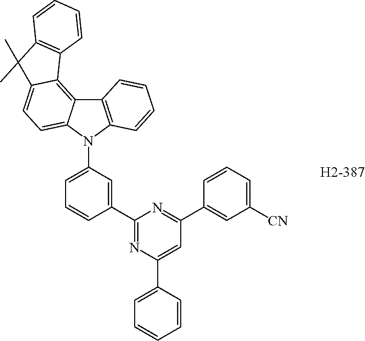 [SMILES: CC1(C)c2ccccc2-c2c1ccc1c2c2ccccc2n1-c1cccc(-c2nc(-c3ccccc3)cc(-c3cccc(C#N)c3)n2)c1]

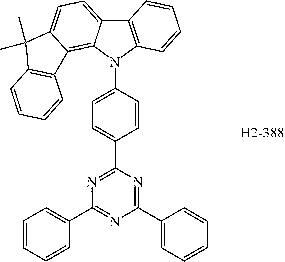 [SMILES: CC1(C)c2ccccc2-c2c1ccc1c3ccccc3n(-c3ccc(-c4nc(-c5ccccc5)nc(-c5ccccc5)n4)cc3)c21]

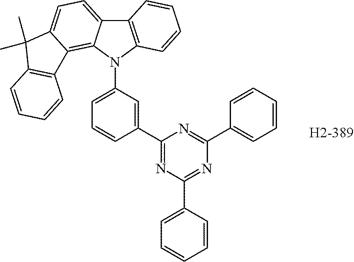 [SMILES: CC1(C)c2ccccc2-c2c1ccc1c3ccccc3n(-c3cccc(-c4nc(-c5ccccc5)nc(-c5ccccc5)n4)c3)c21]

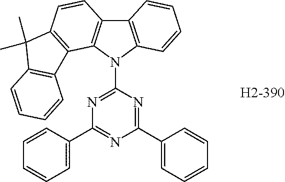 [SMILES: CC1(C)c2ccccc2-c2c1ccc1c3ccccc3n(-c3nc(-c4ccccc4)nc(-c4ccccc4)n3)c21]

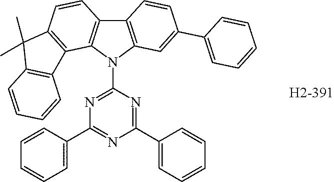 [SMILES: CC1(C)c2ccccc2-c2c1ccc1c3ccc(-c4ccccc4)cc3n(-c3nc(-c4ccccc4)nc(-c4ccccc4)n3)c21]

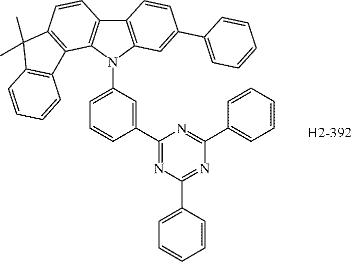 [SMILES: CC1(C)c2ccccc2-c2c1ccc1c3ccc(-c4ccccc4)cc3n(-c3cccc(-c4nc(-c5ccccc5)nc(-c5ccccc5)n4)c3)c21]

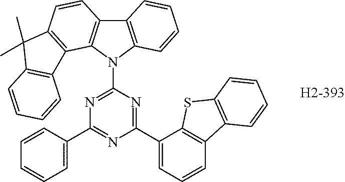 [SMILES: CC1(C)c2ccccc2-c2c1ccc1c3ccccc3n(-c3nc(-c4ccccc4)nc(-c4cccc5c4sc4ccccc45)n3)c21]

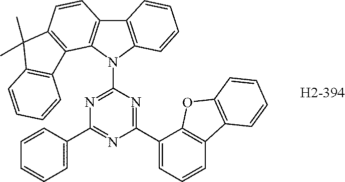 [SMILES: CC1(C)c2ccccc2-c2c1ccc1c3ccccc3n(-c3nc(-c4ccccc4)nc(-c4cccc5c4oc4ccccc45)n3)c21]

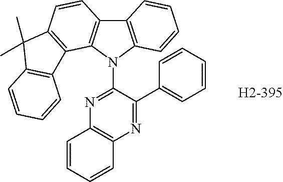 [SMILES: CC1(C)c2ccccc2-c2c1ccc1c3ccccc3n(-c3nc4ccccc4nc3-c3ccccc3)c21]

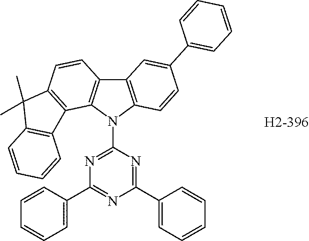 [SMILES: CC1(C)c2ccccc2-c2c1ccc1c3cc(-c4ccccc4)ccc3n(-c3nc(-c4ccccc4)nc(-c4ccccc4)n3)c21]

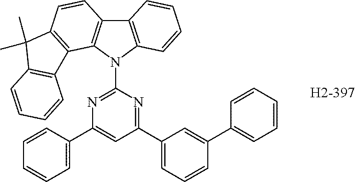 [SMILES: CC1(C)c2ccccc2-c2c1ccc1c3ccccc3n(-c3nc(-c4ccccc4)cc(-c4cccc(-c5ccccc5)c4)n3)c21]

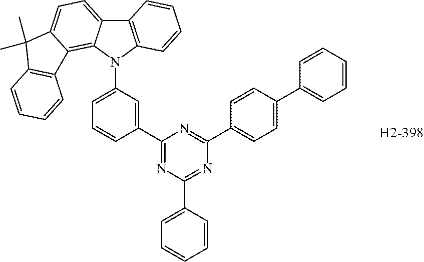 [SMILES: CC1(C)c2ccccc2-c2c1ccc1c3ccccc3n(-c3cccc(-c4nc(-c5ccccc5)nc(-c5ccc(-c6ccccc6)cc5)n4)c3)c21]

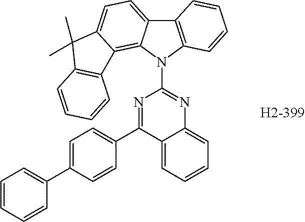 [SMILES: CC1(C)c2ccccc2-c2c1ccc1c3ccccc3n(-c3nc(-c4ccc(-c5ccccc5)cc4)c4ccccc4n3)c21]

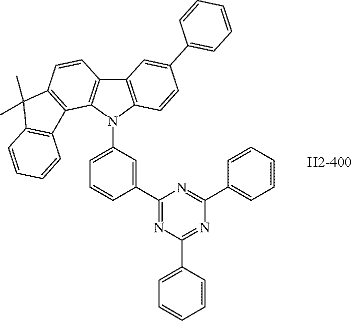 [SMILES: CC1(C)c2ccccc2-c2c1ccc1c3cc(-c4ccccc4)ccc3n(-c3cccc(-c4nc(-c5ccccc5)nc(-c5ccccc5)n4)c3)c21]

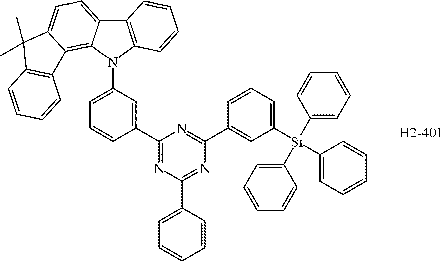 [SMILES: CC1(C)c2ccccc2-c2c1ccc1c3ccccc3n(-c3cccc(-c4nc(-c5ccccc5)nc(-c5cccc([Si](c6ccccc6)(c6ccccc6)c6ccccc6)c5)n4)c3)c21]

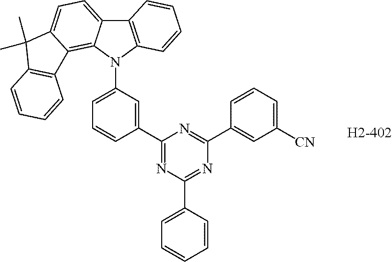 [SMILES: CC1(C)c2ccccc2-c2c1ccc1c3ccccc3n(-c3cccc(-c4nc(-c5ccccc5)nc(-c5cccc(C#N)c5)n4)c3)c21]

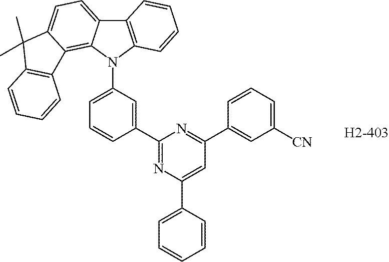 [SMILES: CC1(C)c2ccccc2-c2c1ccc1c3ccccc3n(-c3cccc(-c4nc(-c5ccccc5)cc(-c5cccc(C#N)c5)n4)c3)c21]

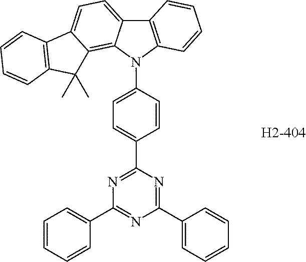 [SMILES: CC1(C)c2ccccc2-c2ccc3c4ccccc4n(-c4ccc(-c5nc(-c6ccccc6)nc(-c6ccccc6)n5)cc4)c3c21]

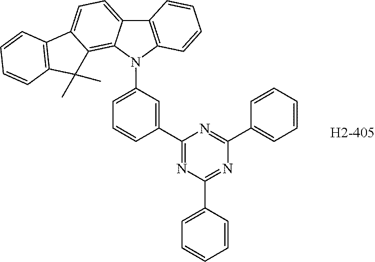 [SMILES: CC1(C)c2ccccc2-c2ccc3c4ccccc4n(-c4cccc(-c5nc(-c6ccccc6)nc(-c6ccccc6)n5)c4)c3c21]

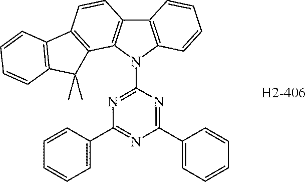 [SMILES: CC1(C)c2ccccc2-c2ccc3c4ccccc4n(-c4nc(-c5ccccc5)nc(-c5ccccc5)n4)c3c21]

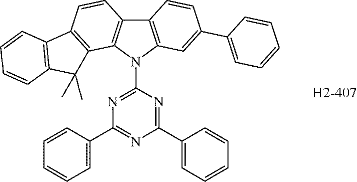 [SMILES: CC1(C)c2ccccc2-c2ccc3c4ccc(-c5ccccc5)cc4n(-c4nc(-c5ccccc5)nc(-c5ccccc5)n4)c3c21]

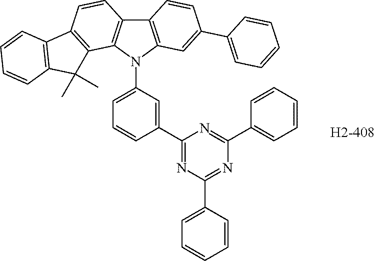 [SMILES: CC1(C)c2ccccc2-c2ccc3c4ccc(-c5ccccc5)cc4n(-c4cccc(-c5nc(-c6ccccc6)nc(-c6ccccc6)n5)c4)c3c21]

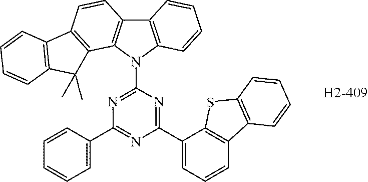 [SMILES: CC1(C)c2ccccc2-c2ccc3c4ccccc4n(-c4nc(-c5ccccc5)nc(-c5cccc6c5sc5ccccc56)n4)c3c21]

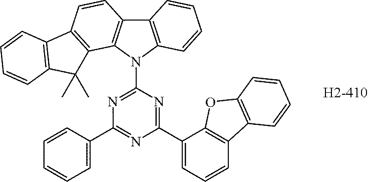 [SMILES: CC1(C)c2ccccc2-c2ccc3c4ccccc4n(-c4nc(-c5ccccc5)nc(-c5cccc6c5oc5ccccc56)n4)c3c21]

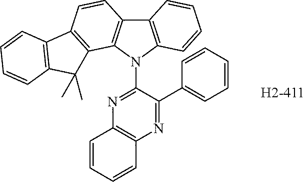 [SMILES: CC1(C)c2ccccc2-c2ccc3c4ccccc4n(-c4nc5ccccc5nc4-c4ccccc4)c3c21]

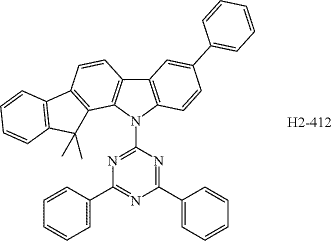 [SMILES: CC1(C)c2ccccc2-c2ccc3c4cc(-c5ccccc5)ccc4n(-c4nc(-c5ccccc5)nc(-c5ccccc5)n4)c3c21]

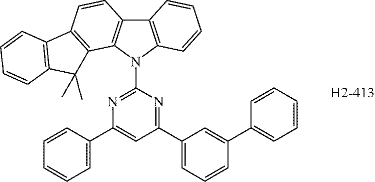 [SMILES: CC1(C)c2ccccc2-c2ccc3c4ccccc4n(-c4nc(-c5ccccc5)cc(-c5cccc(-c6ccccc6)c5)n4)c3c21]

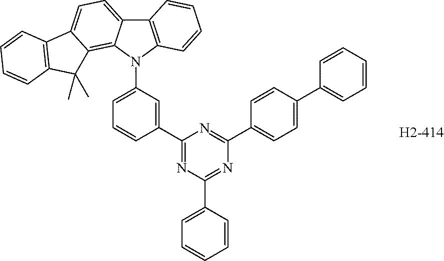 [SMILES: CC1(C)c2ccccc2-c2ccc3c4ccccc4n(-c4cccc(-c5nc(-c6ccccc6)nc(-c6ccc(-c7ccccc7)cc6)n5)c4)c3c21]

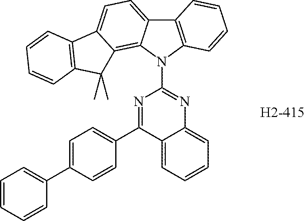 [SMILES: CC1(C)c2ccccc2-c2ccc3c4ccccc4n(-c4nc(-c5ccc(-c6ccccc6)cc5)c5ccccc5n4)c3c21]

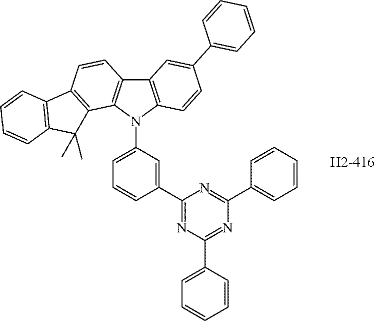 [SMILES: CC1(C)c2ccccc2-c2ccc3c4cc(-c5ccccc5)ccc4n(-c4cccc(-c5nc(-c6ccccc6)nc(-c6ccccc6)n5)c4)c3c21]

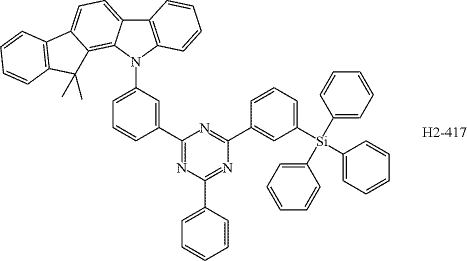 [SMILES: CC1(C)c2ccccc2-c2ccc3c4ccccc4n(-c4cccc(-c5nc(-c6ccccc6)nc(-c6cccc([Si](c7ccccc7)(c7ccccc7)c7ccccc7)c6)n5)c4)c3c21]

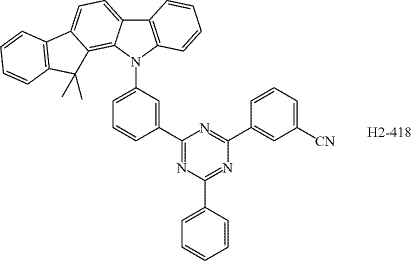 [SMILES: CC1(C)c2ccccc2-c2ccc3c4ccccc4n(-c4cccc(-c5nc(-c6ccccc6)nc(-c6cccc(C#N)c6)n5)c4)c3c21]

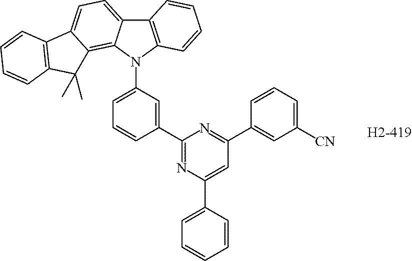 [SMILES: CC1(C)c2ccccc2-c2ccc3c4ccccc4n(-c4cccc(-c5nc(-c6ccccc6)cc(-c6cccc(C#N)c6)n5)c4)c3c21]

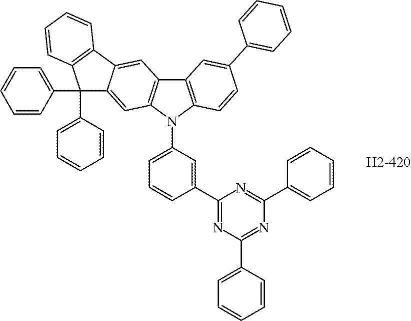 [SMILES: c1ccc(-c2ccc3c(c2)c2cc4c(cc2n3-c2cccc(-c3nc(-c5ccccc5)nc(-c5ccccc5)n3)c2)C(c2ccccc2)(c2ccccc2)c2ccccc2-4)cc1]

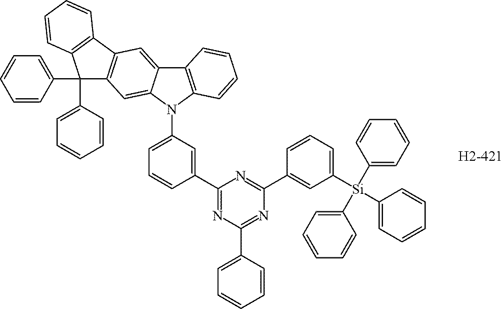 [SMILES: c1ccc(-c2nc(-c3cccc(-n4c5ccccc5c5cc6c(cc54)C(c4ccccc4)(c4ccccc4)c4ccccc4-6)c3)nc(-c3cccc([Si](c4ccccc4)(c4ccccc4)c4ccccc4)c3)n2)cc1]

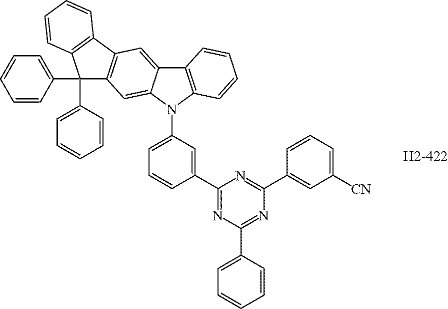 [SMILES: N#Cc1cccc(-c2nc(-c3ccccc3)nc(-c3cccc(-n4c5ccccc5c5cc6c(cc54)C(c4ccccc4)(c4ccccc4)c4ccccc4-6)c3)n2)c1]

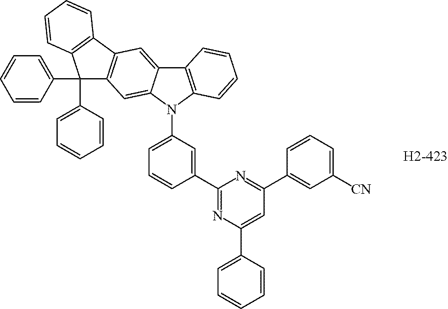 [SMILES: N#Cc1cccc(-c2cc(-c3ccccc3)nc(-c3cccc(-n4c5ccccc5c5cc6c(cc54)C(c4ccccc4)(c4ccccc4)c4ccccc4-6)c3)n2)c1]

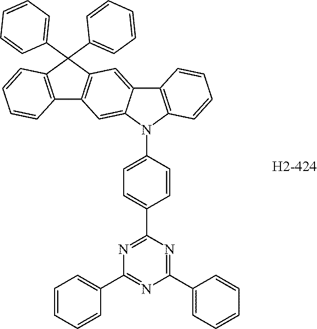 [SMILES: c1ccc(-c2nc(-c3ccccc3)nc(-c3ccc(-n4c5ccccc5c5cc6c(cc54)-c4ccccc4C6(c4ccccc4)c4ccccc4)cc3)n2)cc1]

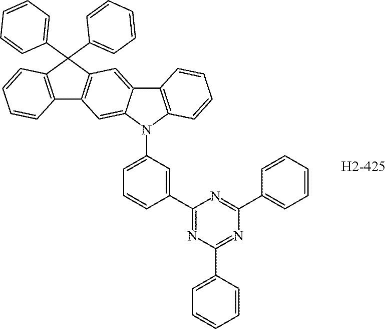 [SMILES: c1ccc(-c2nc(-c3ccccc3)nc(-c3cccc(-n4c5ccccc5c5cc6c(cc54)-c4ccccc4C6(c4ccccc4)c4ccccc4)c3)n2)cc1]

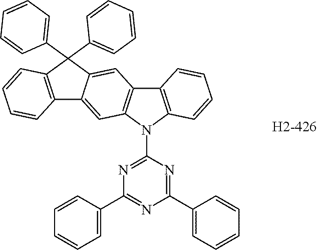 [SMILES: c1ccc(-c2nc(-c3ccccc3)nc(-n3c4ccccc4c4cc5c(cc43)-c3ccccc3C5(c3ccccc3)c3ccccc3)n2)cc1]

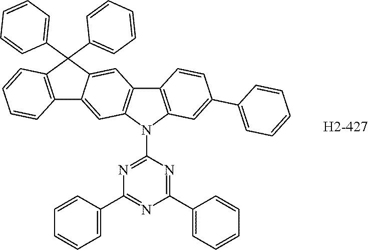 [SMILES: c1ccc(-c2ccc3c4cc5c(cc4n(-c4nc(-c6ccccc6)nc(-c6ccccc6)n4)c3c2)-c2ccccc2C5(c2ccccc2)c2ccccc2)cc1]

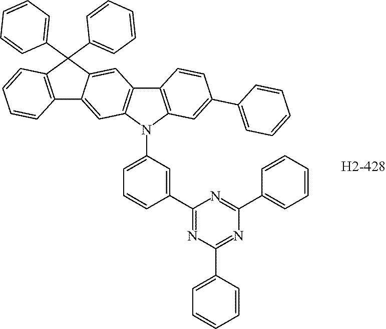 [SMILES: c1ccc(-c2ccc3c4cc5c(cc4n(-c4cccc(-c6nc(-c7ccccc7)nc(-c7ccccc7)n6)c4)c3c2)-c2ccccc2C5(c2ccccc2)c2ccccc2)cc1]

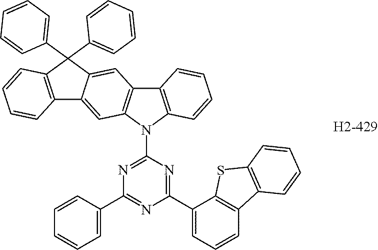 [SMILES: c1ccc(-c2nc(-c3cccc4c3sc3ccccc34)nc(-n3c4ccccc4c4cc5c(cc43)-c3ccccc3C5(c3ccccc3)c3ccccc3)n2)cc1]

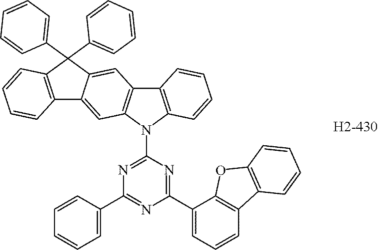 [SMILES: c1ccc(-c2nc(-c3cccc4c3oc3ccccc34)nc(-n3c4ccccc4c4cc5c(cc43)-c3ccccc3C5(c3ccccc3)c3ccccc3)n2)cc1]

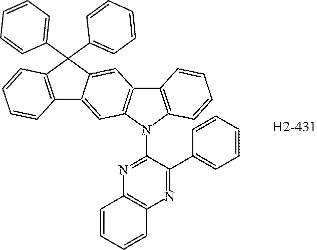 [SMILES: c1ccc(-c2nc3ccccc3nc2-n2c3ccccc3c3cc4c(cc32)-c2ccccc2C4(c2ccccc2)c2ccccc2)cc1]

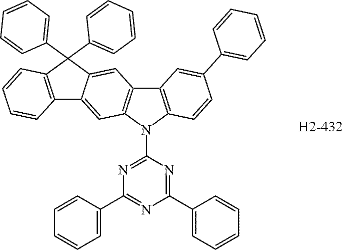 [SMILES: c1ccc(-c2ccc3c(c2)c2cc4c(cc2n3-c2nc(-c3ccccc3)nc(-c3ccccc3)n2)-c2ccccc2C4(c2ccccc2)c2ccccc2)cc1]

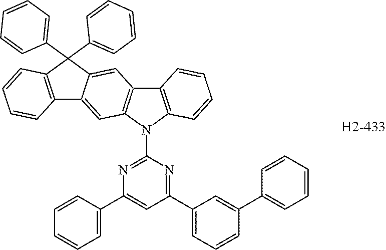 [SMILES: c1ccc(-c2cccc(-c3cc(-c4ccccc4)nc(-n4c5ccccc5c5cc6c(cc54)-c4ccccc4C6(c4ccccc4)c4ccccc4)n3)c2)cc1]

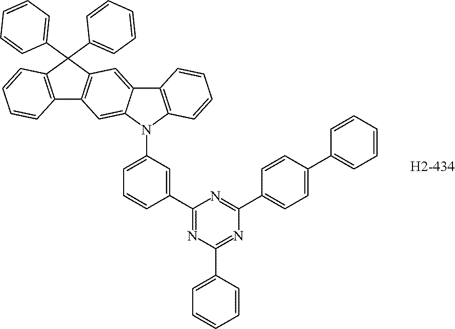 [SMILES: c1ccc(-c2ccc(-c3nc(-c4ccccc4)nc(-c4cccc(-n5c6ccccc6c6cc7c(cc65)-c5ccccc5C7(c5ccccc5)c5ccccc5)c4)n3)cc2)cc1]

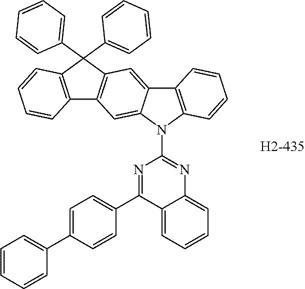 [SMILES: c1ccc(-c2ccc(-c3nc(-n4c5ccccc5c5cc6c(cc54)-c4ccccc4C6(c4ccccc4)c4ccccc4)nc4ccccc34)cc2)cc1]

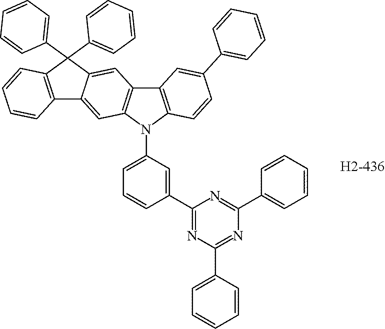 [SMILES: c1ccc(-c2ccc3c(c2)c2cc4c(cc2n3-c2cccc(-c3nc(-c5ccccc5)nc(-c5ccccc5)n3)c2)-c2ccccc2C4(c2ccccc2)c2ccccc2)cc1]

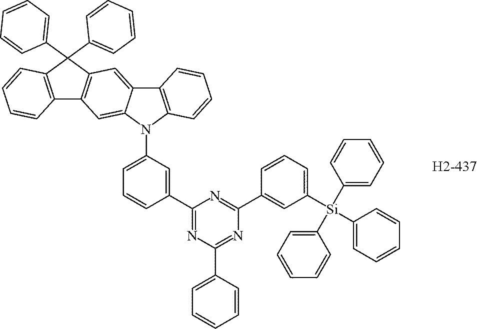 [SMILES: c1ccc(-c2nc(-c3cccc(-n4c5ccccc5c5cc6c(cc54)-c4ccccc4C6(c4ccccc4)c4ccccc4)c3)nc(-c3cccc([Si](c4ccccc4)(c4ccccc4)c4ccccc4)c3)n2)cc1]

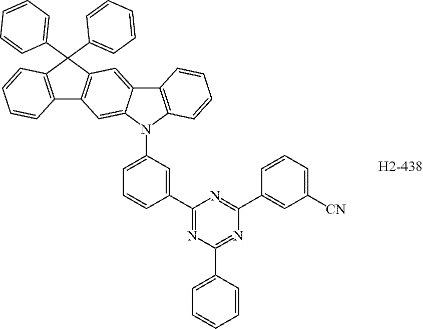 [SMILES: N#Cc1cccc(-c2nc(-c3ccccc3)nc(-c3cccc(-n4c5ccccc5c5cc6c(cc54)-c4ccccc4C6(c4ccccc4)c4ccccc4)c3)n2)c1]

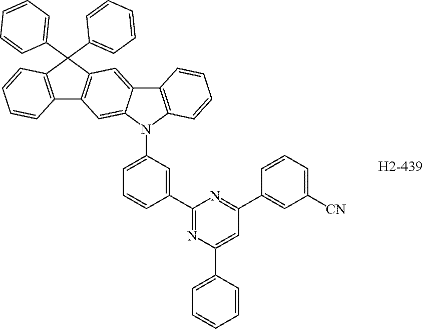 [SMILES: N#Cc1cccc(-c2cc(-c3ccccc3)nc(-c3cccc(-n4c5ccccc5c5cc6c(cc54)-c4ccccc4C6(c4ccccc4)c4ccccc4)c3)n2)c1]

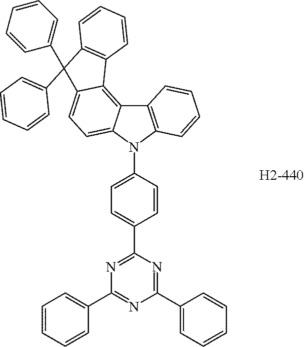 [SMILES: c1ccc(-c2nc(-c3ccccc3)nc(-c3ccc(-n4c5ccccc5c5c6c(ccc54)C(c4ccccc4)(c4ccccc4)c4ccccc4-6)cc3)n2)cc1]

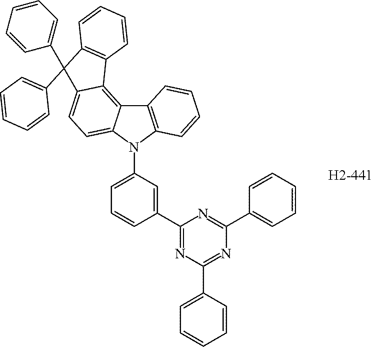 [SMILES: c1ccc(-c2nc(-c3ccccc3)nc(-c3cccc(-n4c5ccccc5c5c6c(ccc54)C(c4ccccc4)(c4ccccc4)c4ccccc4-6)c3)n2)cc1]

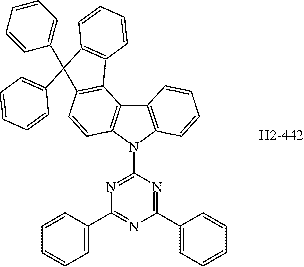 [SMILES: c1ccc(-c2nc(-c3ccccc3)nc(-n3c4ccccc4c4c5c(ccc43)C(c3ccccc3)(c3ccccc3)c3ccccc3-5)n2)cc1]

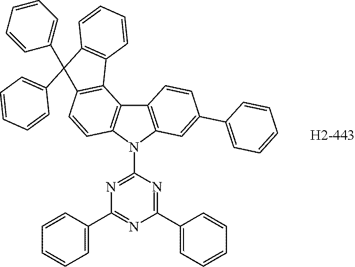 [SMILES: c1ccc(-c2ccc3c4c5c(ccc4n(-c4nc(-c6ccccc6)nc(-c6ccccc6)n4)c3c2)C(c2ccccc2)(c2ccccc2)c2ccccc2-5)cc1]

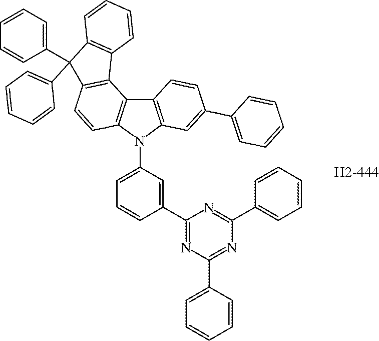 [SMILES: c1ccc(-c2ccc3c4c5c(ccc4n(-c4cccc(-c6nc(-c7ccccc7)nc(-c7ccccc7)n6)c4)c3c2)C(c2ccccc2)(c2ccccc2)c2ccccc2-5)cc1]

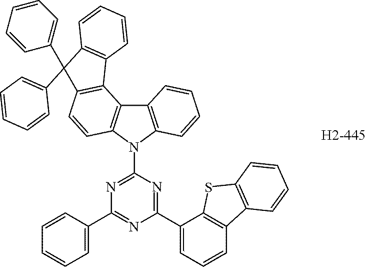 [SMILES: c1ccc(-c2nc(-c3cccc4c3sc3ccccc34)nc(-n3c4ccccc4c4c5c(ccc43)C(c3ccccc3)(c3ccccc3)c3ccccc3-5)n2)cc1]

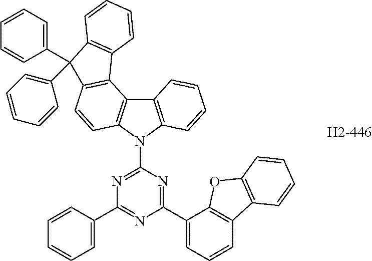 [SMILES: c1ccc(-c2nc(-c3cccc4c3oc3ccccc34)nc(-n3c4ccccc4c4c5c(ccc43)C(c3ccccc3)(c3ccccc3)c3ccccc3-5)n2)cc1]

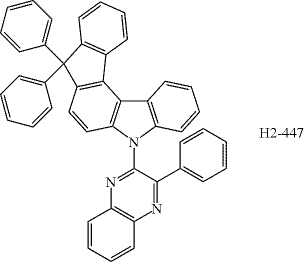 [SMILES: c1ccc(-c2nc3ccccc3nc2-n2c3ccccc3c3c4c(ccc32)C(c2ccccc2)(c2ccccc2)c2ccccc2-4)cc1]

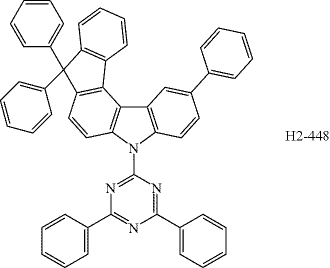 [SMILES: c1ccc(-c2ccc3c(c2)c2c4c(ccc2n3-c2nc(-c3ccccc3)nc(-c3ccccc3)n2)C(c2ccccc2)(c2ccccc2)c2ccccc2-4)cc1]

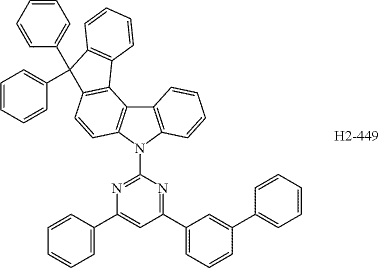 [SMILES: c1ccc(-c2cccc(-c3cc(-c4ccccc4)nc(-n4c5ccccc5c5c6c(ccc54)C(c4ccccc4)(c4ccccc4)c4ccccc4-6)n3)c2)cc1]

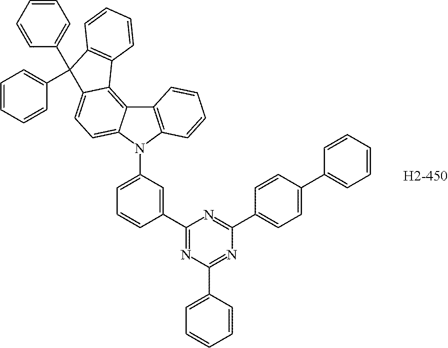 [SMILES: c1ccc(-c2ccc(-c3nc(-c4ccccc4)nc(-c4cccc(-n5c6ccccc6c6c7c(ccc65)C(c5ccccc5)(c5ccccc5)c5ccccc5-7)c4)n3)cc2)cc1]

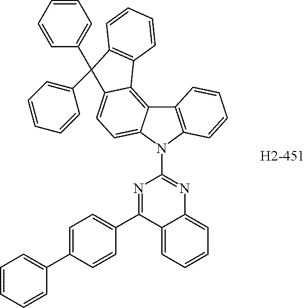 [SMILES: c1ccc(-c2ccc(-c3nc(-n4c5ccccc5c5c6c(ccc54)C(c4ccccc4)(c4ccccc4)c4ccccc4-6)nc4ccccc34)cc2)cc1]

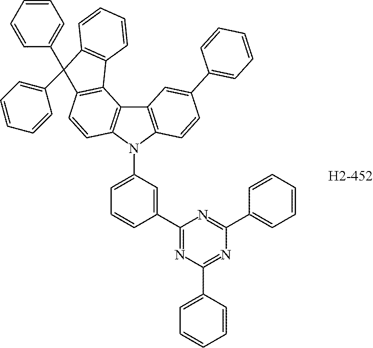 [SMILES: c1ccc(-c2ccc3c(c2)c2c4c(ccc2n3-c2cccc(-c3nc(-c5ccccc5)nc(-c5ccccc5)n3)c2)C(c2ccccc2)(c2ccccc2)c2ccccc2-4)cc1]

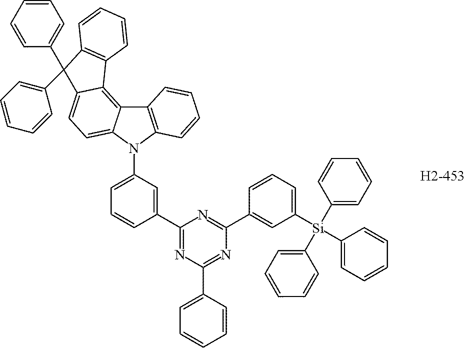 [SMILES: c1ccc(-c2nc(-c3cccc(-n4c5ccccc5c5c6c(ccc54)C(c4ccccc4)(c4ccccc4)c4ccccc4-6)c3)nc(-c3cccc([Si](c4ccccc4)(c4ccccc4)c4ccccc4)c3)n2)cc1]

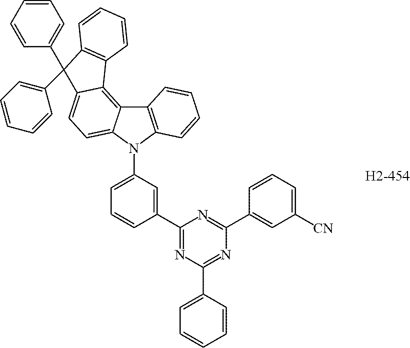 [SMILES: N#Cc1cccc(-c2nc(-c3ccccc3)nc(-c3cccc(-n4c5ccccc5c5c6c(ccc54)C(c4ccccc4)(c4ccccc4)c4ccccc4-6)c3)n2)c1]

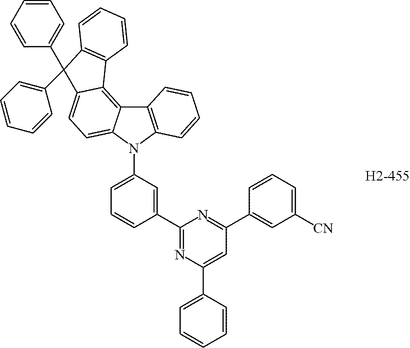 [SMILES: N#Cc1cccc(-c2cc(-c3ccccc3)nc(-c3cccc(-n4c5ccccc5c5c6c(ccc54)C(c4ccccc4)(c4ccccc4)c4ccccc4-6)c3)n2)c1]

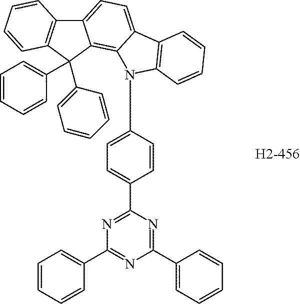 [SMILES: c1ccc(-c2nc(-c3ccccc3)nc(-c3ccc(-n4c5ccccc5c5ccc6c(c54)C(c4ccccc4)(c4ccccc4)c4ccccc4-6)cc3)n2)cc1]

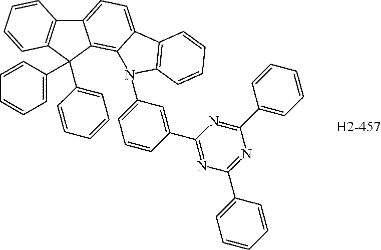 [SMILES: c1ccc(-c2nc(-c3ccccc3)nc(-c3cccc(-n4c5ccccc5c5ccc6c(c54)C(c4ccccc4)(c4ccccc4)c4ccccc4-6)c3)n2)cc1]

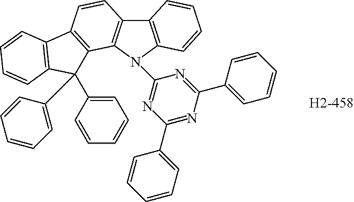 [SMILES: c1ccc(-c2nc(-c3ccccc3)nc(-n3c4ccccc4c4ccc5c(c43)C(c3ccccc3)(c3ccccc3)c3ccccc3-5)n2)cc1]

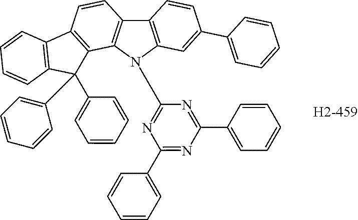 [SMILES: c1ccc(-c2ccc3c4ccc5c(c4n(-c4nc(-c6ccccc6)nc(-c6ccccc6)n4)c3c2)C(c2ccccc2)(c2ccccc2)c2ccccc2-5)cc1]

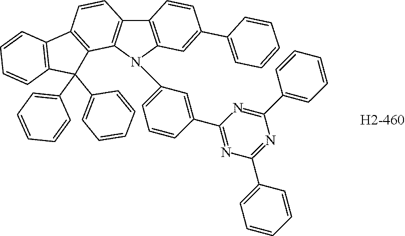 [SMILES: c1ccc(-c2ccc3c4ccc5c(c4n(-c4cccc(-c6nc(-c7ccccc7)nc(-c7ccccc7)n6)c4)c3c2)C(c2ccccc2)(c2ccccc2)c2ccccc2-5)cc1]

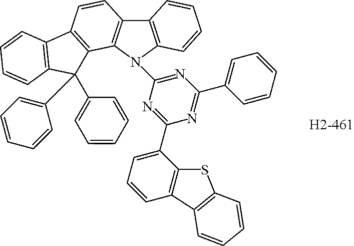 [SMILES: c1ccc(-c2nc(-c3cccc4c3sc3ccccc34)nc(-n3c4ccccc4c4ccc5c(c43)C(c3ccccc3)(c3ccccc3)c3ccccc3-5)n2)cc1]

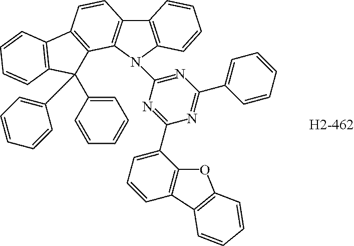 [SMILES: c1ccc(-c2nc(-c3cccc4c3oc3ccccc34)nc(-n3c4ccccc4c4ccc5c(c43)C(c3ccccc3)(c3ccccc3)c3ccccc3-5)n2)cc1]

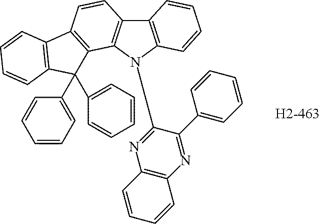 [SMILES: c1ccc(-c2nc3ccccc3nc2-n2c3ccccc3c3ccc4c(c32)C(c2ccccc2)(c2ccccc2)c2ccccc2-4)cc1]

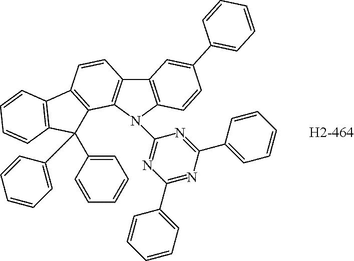 [SMILES: c1ccc(-c2ccc3c(c2)c2ccc4c(c2n3-c2nc(-c3ccccc3)nc(-c3ccccc3)n2)C(c2ccccc2)(c2ccccc2)c2ccccc2-4)cc1]

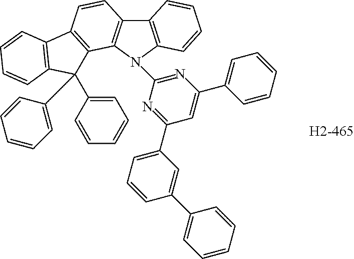 [SMILES: c1ccc(-c2cccc(-c3cc(-c4ccccc4)nc(-n4c5ccccc5c5ccc6c(c54)C(c4ccccc4)(c4ccccc4)c4ccccc4-6)n3)c2)cc1]

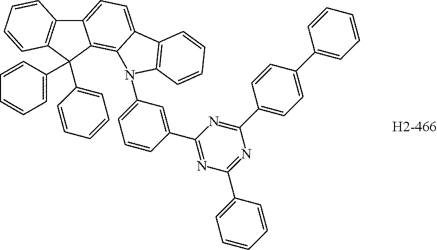 [SMILES: c1ccc(-c2ccc(-c3nc(-c4ccccc4)nc(-c4cccc(-n5c6ccccc6c6ccc7c(c65)C(c5ccccc5)(c5ccccc5)c5ccccc5-7)c4)n3)cc2)cc1]

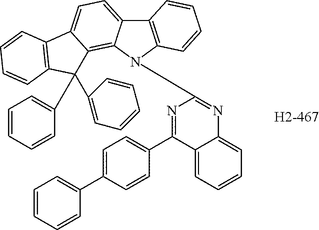 [SMILES: c1ccc(-c2ccc(-c3nc(-n4c5ccccc5c5ccc6c(c54)C(c4ccccc4)(c4ccccc4)c4ccccc4-6)nc4ccccc34)cc2)cc1]

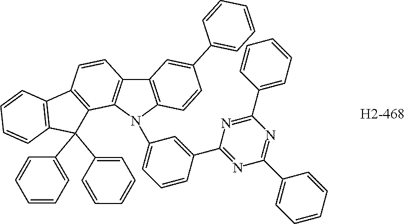 [SMILES: c1ccc(-c2ccc3c(c2)c2ccc4c(c2n3-c2cccc(-c3nc(-c5ccccc5)nc(-c5ccccc5)n3)c2)C(c2ccccc2)(c2ccccc2)c2ccccc2-4)cc1]

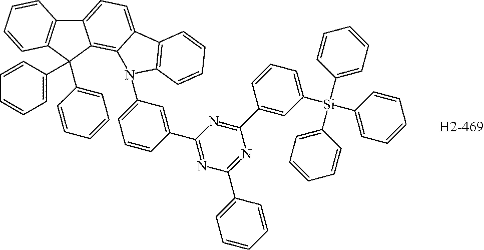 [SMILES: c1ccc(-c2nc(-c3cccc(-n4c5ccccc5c5ccc6c(c54)C(c4ccccc4)(c4ccccc4)c4ccccc4-6)c3)nc(-c3cccc([Si](c4ccccc4)(c4ccccc4)c4ccccc4)c3)n2)cc1]

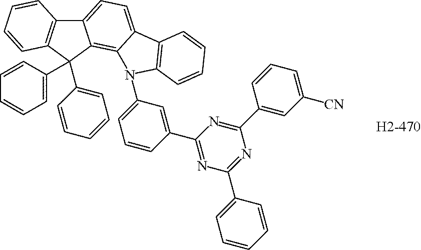 [SMILES: N#Cc1cccc(-c2nc(-c3ccccc3)nc(-c3cccc(-n4c5ccccc5c5ccc6c(c54)C(c4ccccc4)(c4ccccc4)c4ccccc4-6)c3)n2)c1]

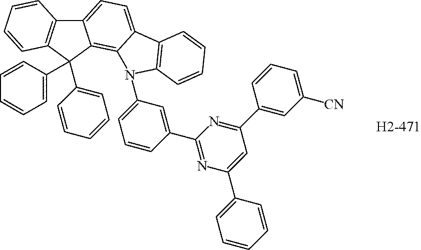 [SMILES: N#Cc1cccc(-c2cc(-c3ccccc3)nc(-c3cccc(-n4c5ccccc5c5ccc6c(c54)C(c4ccccc4)(c4ccccc4)c4ccccc4-6)c3)n2)c1]

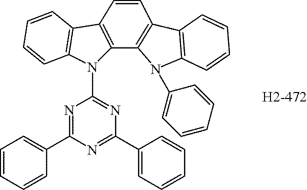 [SMILES: c1ccc(-c2nc(-c3ccccc3)nc(-n3c4ccccc4c4ccc5c6ccccc6n(-c6ccccc6)c5c43)n2)cc1]

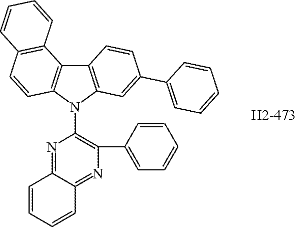 [SMILES: c1ccc(-c2ccc3c4c5ccccc5ccc4n(-c4nc5ccccc5nc4-c4ccccc4)c3c2)cc1]

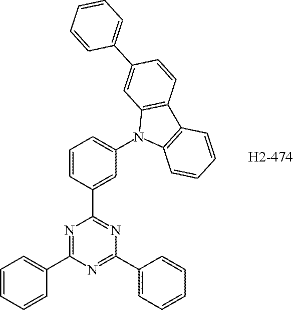 [SMILES: c1ccc(-c2ccc3c4ccccc4n(-c4cccc(-c5nc(-c6ccccc6)nc(-c6ccccc6)n5)c4)c3c2)cc1]

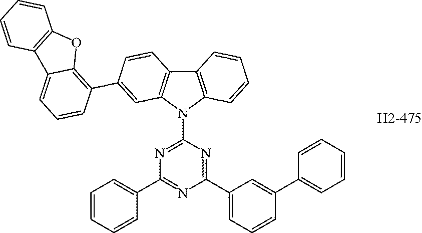 [SMILES: c1ccc(-c2cccc(-c3nc(-c4ccccc4)nc(-n4c5ccccc5c5ccc(-c6cccc7c6oc6ccccc67)cc54)n3)c2)cc1]

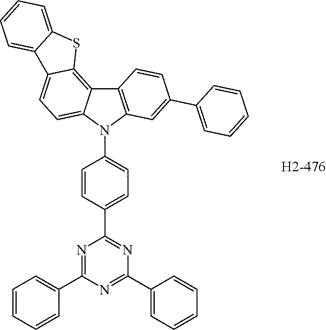 [SMILES: c1ccc(-c2ccc3c4c5sc6ccccc6c5ccc4n(-c4ccc(-c5nc(-c6ccccc6)nc(-c6ccccc6)n5)cc4)c3c2)cc1]

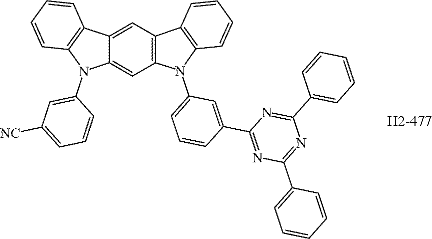

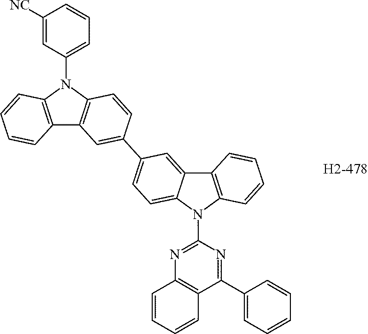 [SMILES: N#Cc1cccc(-n2c3ccccc3c3cc(-c4ccc5c(c4)c4ccccc4n5-c4nc(-c5ccccc5)c5ccccc5n4)ccc32)c1]

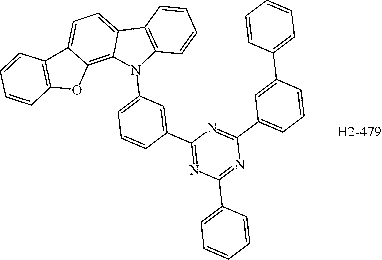 [SMILES: c1ccc(-c2cccc(-c3nc(-c4ccccc4)nc(-c4cccc(-n5c6ccccc6c6ccc7c8ccccc8oc7c65)c4)n3)c2)cc1]

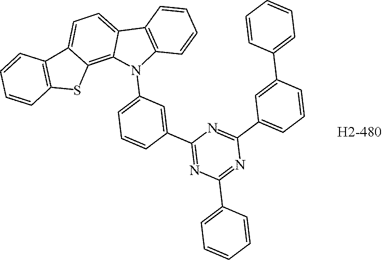 [SMILES: c1ccc(-c2cccc(-c3nc(-c4ccccc4)nc(-c4cccc(-n5c6ccccc6c6ccc7c8ccccc8sc7c65)c4)n3)c2)cc1]

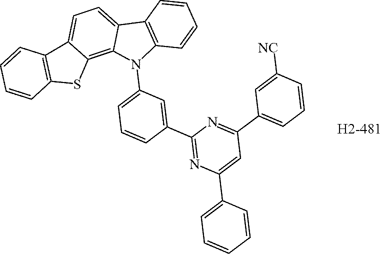

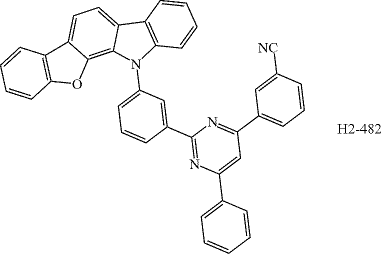

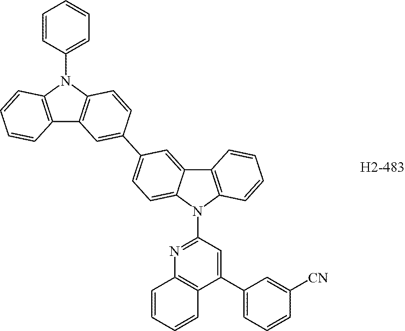 [SMILES: N#Cc1cccc(-c2cc(-n3c4ccccc4c4cc(-c5ccc6c(c5)c5ccccc5n6-c5ccccc5)ccc43)nc3ccccc23)c1]

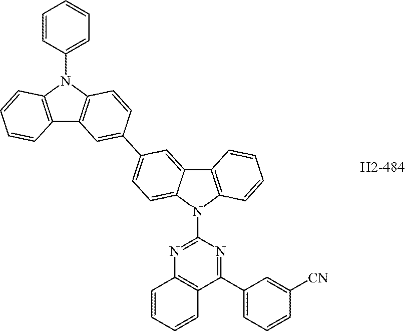 [SMILES: N#Cc1cccc(-c2nc(-n3c4ccccc4c4cc(-c5ccc6c(c5)c5ccccc5n6-c5ccccc5)ccc43)nc3ccccc23)c1]

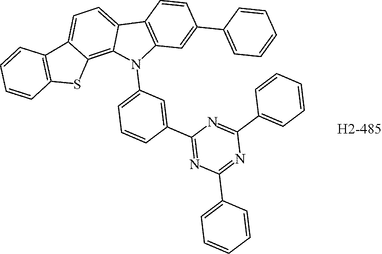 [SMILES: c1ccc(-c2ccc3c4ccc5c6ccccc6sc5c4n(-c4cccc(-c5nc(-c6ccccc6)nc(-c6ccccc6)n5)c4)c3c2)cc1]

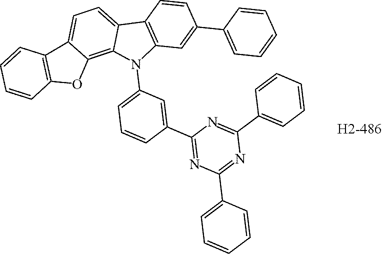 [SMILES: c1ccc(-c2ccc3c4ccc5c6ccccc6oc5c4n(-c4cccc(-c5nc(-c6ccccc6)nc(-c6ccccc6)n5)c4)c3c2)cc1]

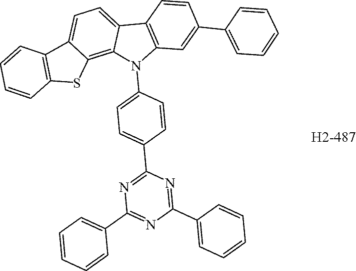 [SMILES: c1ccc(-c2ccc3c4ccc5c6ccccc6sc5c4n(-c4ccc(-c5nc(-c6ccccc6)nc(-c6ccccc6)n5)cc4)c3c2)cc1]

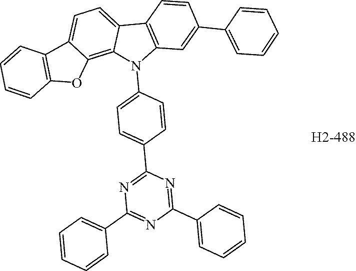 [SMILES: c1ccc(-c2ccc3c4ccc5c6ccccc6oc5c4n(-c4ccc(-c5nc(-c6ccccc6)nc(-c6ccccc6)n5)cc4)c3c2)cc1]

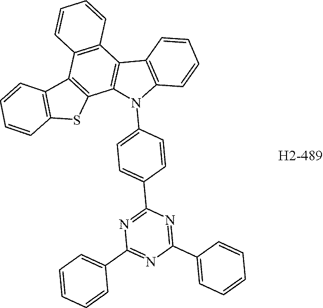 [SMILES: c1ccc(-c2nc(-c3ccccc3)nc(-c3ccc(-n4c5ccccc5c5c6ccccc6c6c7ccccc7sc6c54)cc3)n2)cc1]

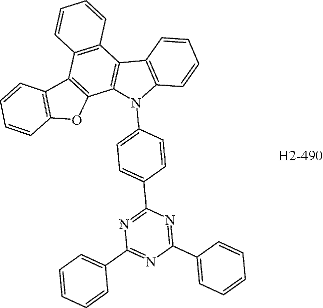 [SMILES: c1ccc(-c2nc(-c3ccccc3)nc(-c3ccc(-n4c5ccccc5c5c6ccccc6c6c7ccccc7oc6c54)cc3)n2)cc1]

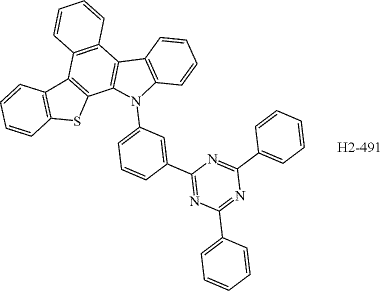 [SMILES: c1ccc(-c2nc(-c3ccccc3)nc(-c3cccc(-n4c5ccccc5c5c6ccccc6c6c7ccccc7sc6c54)c3)n2)cc1]

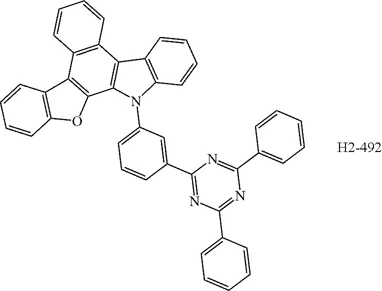 [SMILES: c1ccc(-c2nc(-c3ccccc3)nc(-c3cccc(-n4c5ccccc5c5c6ccccc6c6c7ccccc7oc6c54)c3)n2)cc1]

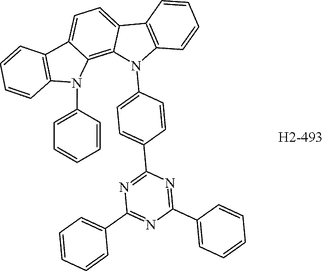 [SMILES: c1ccc(-c2nc(-c3ccccc3)nc(-c3ccc(-n4c5ccccc5c5ccc6c7ccccc7n(-c7ccccc7)c6c54)cc3)n2)cc1]

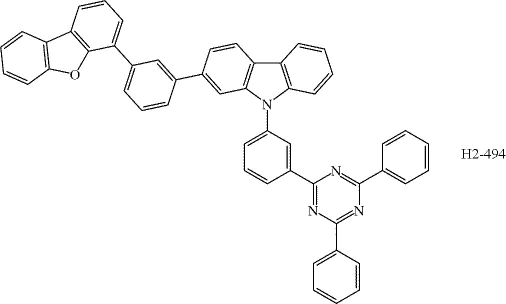 [SMILES: c1ccc(-c2nc(-c3ccccc3)nc(-c3cccc(-n4c5ccccc5c5ccc(-c6cccc(-c7cccc8c7oc7ccccc78)c6)cc54)c3)n2)cc1]

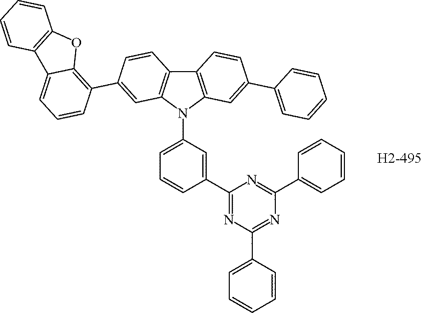 [SMILES: c1ccc(-c2ccc3c4ccc(-c5cccc6c5oc5ccccc56)cc4n(-c4cccc(-c5nc(-c6ccccc6)nc(-c6ccccc6)n5)c4)c3c2)cc1]

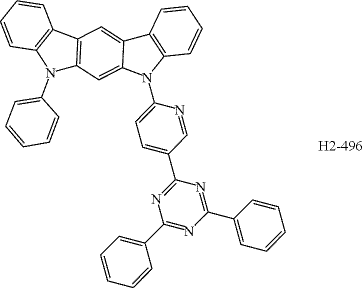 [SMILES: c1ccc(-c2nc(-c3ccccc3)nc(-c3ccc(-n4c5ccccc5c5cc6c7ccccc7n(-c7ccccc7)c6cc54)nc3)n2)cc1]

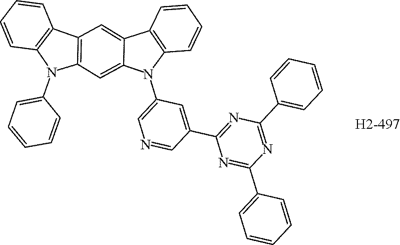 [SMILES: c1ccc(-c2nc(-c3ccccc3)nc(-c3cncc(-n4c5ccccc5c5cc6c7ccccc7n(-c7ccccc7)c6cc54)c3)n2)cc1]

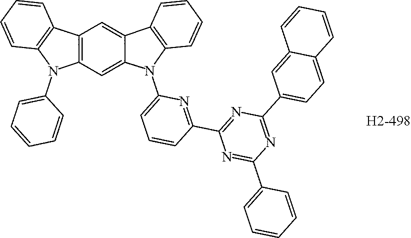 [SMILES: c1ccc(-c2nc(-c3ccc4ccccc4c3)nc(-c3cccc(-n4c5ccccc5c5cc6c7ccccc7n(-c7ccccc7)c6cc54)n3)n2)cc1]

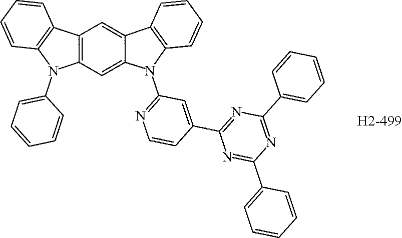 [SMILES: c1ccc(-c2nc(-c3ccccc3)nc(-c3ccnc(-n4c5ccccc5c5cc6c7ccccc7n(-c7ccccc7)c6cc54)c3)n2)cc1]

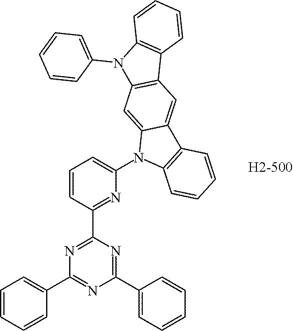 [SMILES: c1ccc(-c2nc(-c3ccccc3)nc(-c3cccc(-n4c5ccccc5c5cc6c7ccccc7n(-c7ccccc7)c6cc54)n3)n2)cc1]

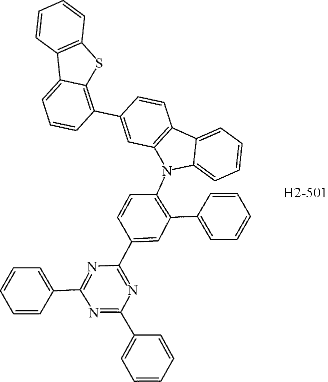 [SMILES: c1ccc(-c2nc(-c3ccccc3)nc(-c3ccc(-n4c5ccccc5c5ccc(-c6cccc7c6sc6ccccc67)cc54)c(-c4ccccc4)c3)n2)cc1]

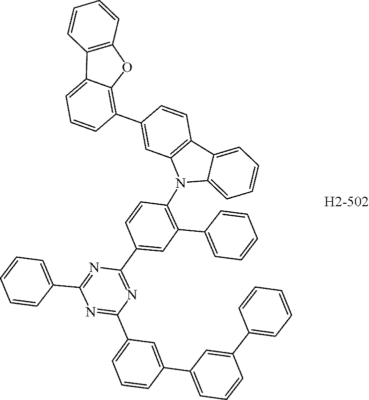 [SMILES: c1ccc(-c2cccc(-c3cccc(-c4nc(-c5ccccc5)nc(-c5ccc(-n6c7ccccc7c7ccc(-c8cccc9c8oc8ccccc89)cc76)c(-c6ccccc6)c5)n4)c3)c2)cc1]

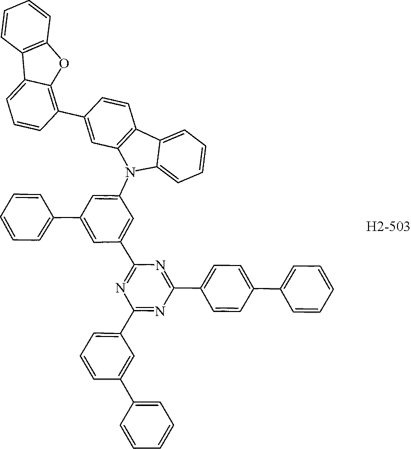 [SMILES: c1ccc(-c2ccc(-c3nc(-c4cccc(-c5ccccc5)c4)nc(-c4cc(-c5ccccc5)cc(-n5c6ccccc6c6ccc(-c7cccc8c7oc7ccccc78)cc65)c4)n3)cc2)cc1]

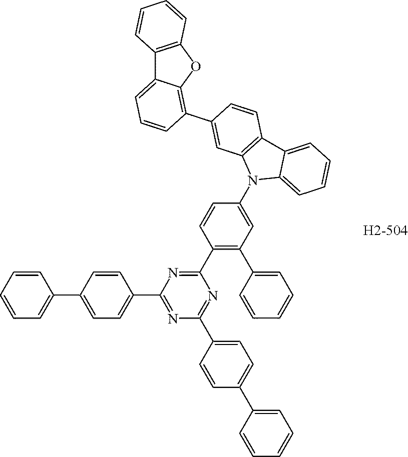 [SMILES: c1ccc(-c2ccc(-c3nc(-c4ccc(-c5ccccc5)cc4)nc(-c4ccc(-n5c6ccccc6c6ccc(-c7cccc8c7oc7ccccc78)cc65)cc4-c4ccccc4)n3)cc2)cc1]

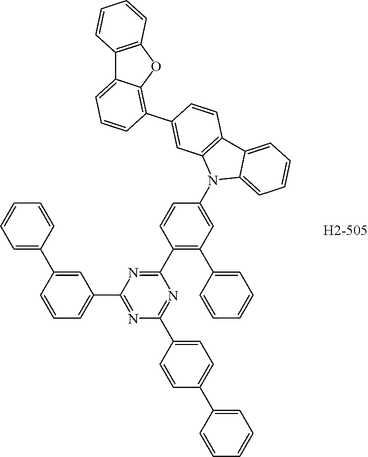 [SMILES: c1ccc(-c2ccc(-c3nc(-c4cccc(-c5ccccc5)c4)nc(-c4ccc(-n5c6ccccc6c6ccc(-c7cccc8c7oc7ccccc78)cc65)cc4-c4ccccc4)n3)cc2)cc1]

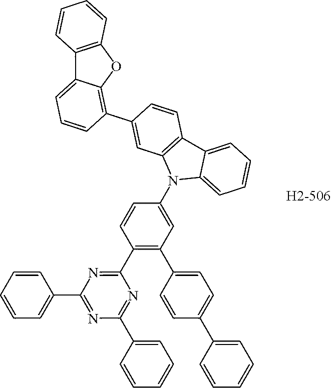 [SMILES: c1ccc(-c2ccc(-c3cc(-n4c5ccccc5c5ccc(-c6cccc7c6oc6ccccc67)cc54)ccc3-c3nc(-c4ccccc4)nc(-c4ccccc4)n3)cc2)cc1]

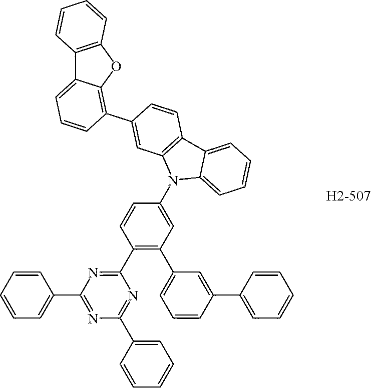 [SMILES: c1ccc(-c2cccc(-c3cc(-n4c5ccccc5c5ccc(-c6cccc7c6oc6ccccc67)cc54)ccc3-c3nc(-c4ccccc4)nc(-c4ccccc4)n3)c2)cc1]

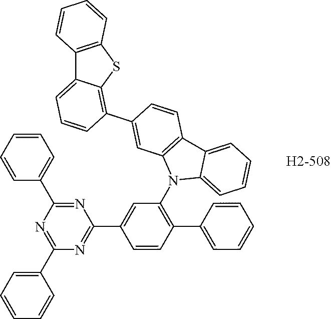 [SMILES: c1ccc(-c2nc(-c3ccccc3)nc(-c3ccc(-c4ccccc4)c(-n4c5ccccc5c5ccc(-c6cccc7c6sc6ccccc67)cc54)c3)n2)cc1]

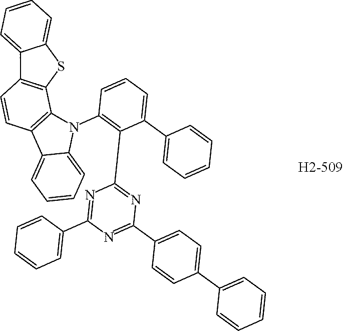 [SMILES: c1ccc(-c2ccc(-c3nc(-c4ccccc4)nc(-c4c(-c5ccccc5)cccc4-n4c5ccccc5c5ccc6c7ccccc7sc6c54)n3)cc2)cc1]

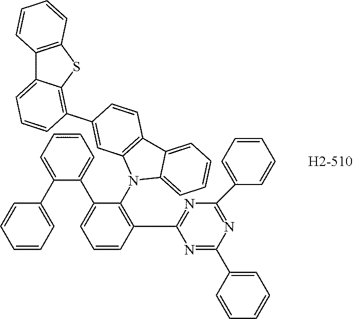 [SMILES: c1ccc(-c2nc(-c3ccccc3)nc(-c3cccc(-c4ccccc4-c4ccccc4)c3-n3c4ccccc4c4ccc(-c5cccc6c5sc5ccccc56)cc43)n2)cc1]

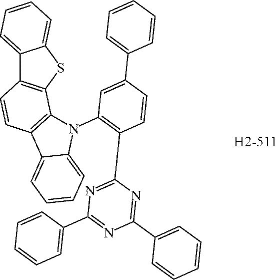 [SMILES: c1ccc(-c2ccc(-c3nc(-c4ccccc4)nc(-c4ccccc4)n3)c(-n3c4ccccc4c4ccc5c6ccccc6sc5c43)c2)cc1]

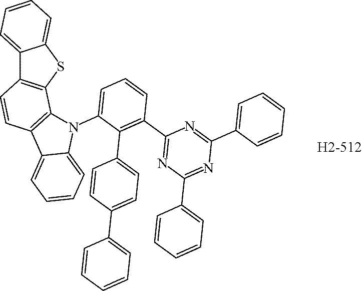 [SMILES: c1ccc(-c2ccc(-c3c(-c4nc(-c5ccccc5)nc(-c5ccccc5)n4)cccc3-n3c4ccccc4c4ccc5c6ccccc6sc5c43)cc2)cc1]

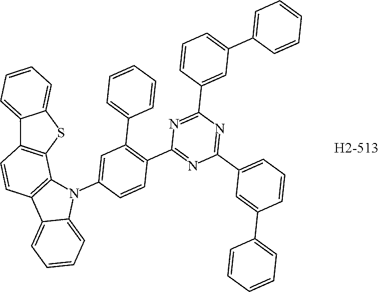 [SMILES: c1ccc(-c2cccc(-c3nc(-c4cccc(-c5ccccc5)c4)nc(-c4ccc(-n5c6ccccc6c6ccc7c8ccccc8sc7c65)cc4-c4ccccc4)n3)c2)cc1]

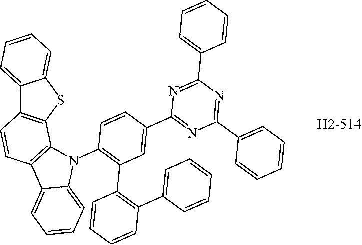 [SMILES: c1ccc(-c2nc(-c3ccccc3)nc(-c3ccc(-n4c5ccccc5c5ccc6c7ccccc7sc6c54)c(-c4ccccc4-c4ccccc4)c3)n2)cc1]

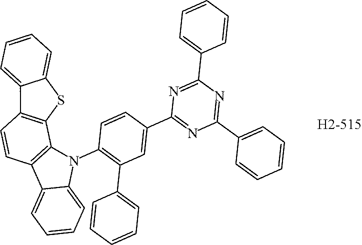 [SMILES: c1ccc(-c2nc(-c3ccccc3)nc(-c3ccc(-n4c5ccccc5c5ccc6c7ccccc7sc6c54)c(-c4ccccc4)c3)n2)cc1]

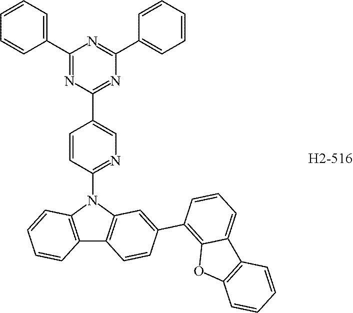 [SMILES: c1ccc(-c2nc(-c3ccccc3)nc(-c3ccc(-n4c5ccccc5c5ccc(-c6cccc7c6oc6ccccc67)cc54)nc3)n2)cc1]

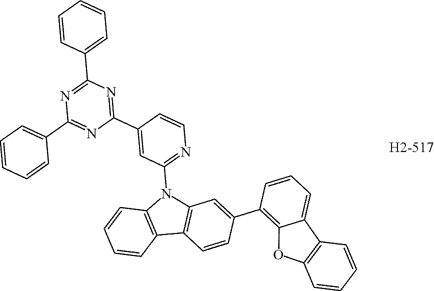 [SMILES: c1ccc(-c2nc(-c3ccccc3)nc(-c3ccnc(-n4c5ccccc5c5ccc(-c6cccc7c6oc6ccccc67)cc54)c3)n2)cc1]

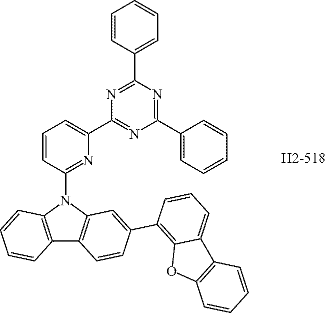 [SMILES: c1ccc(-c2nc(-c3ccccc3)nc(-c3cccc(-n4c5ccccc5c5ccc(-c6cccc7c6oc6ccccc67)cc54)n3)n2)cc1]

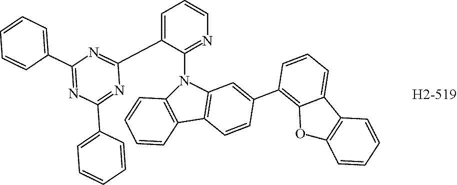 [SMILES: c1ccc(-c2nc(-c3ccccc3)nc(-c3cccnc3-n3c4ccccc4c4ccc(-c5cccc6c5oc5ccccc56)cc43)n2)cc1]

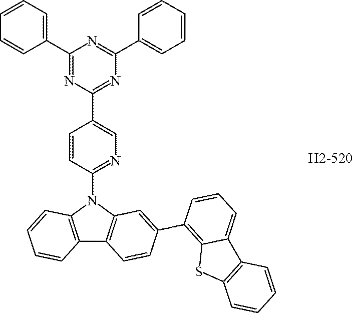 [SMILES: c1ccc(-c2nc(-c3ccccc3)nc(-c3ccc(-n4c5ccccc5c5ccc(-c6cccc7c6sc6ccccc67)cc54)nc3)n2)cc1]

The combination of at least one of compounds C-1 to C-233 and at least one of compounds H2-1 to H2-520 may be used in an organic electroluminescent device.

The present disclosure provides the compounds represented by formulas 1 to 18. The present disclosure may provide an organic electroluminescent material or an organic electroluminescent device comprising the organic electroluminescent compound, in which the organic electroluminescent compound may be comprised as a host material in a light-emitting layer, or as an electron transport material in an electron transport layer. According to one embodiment of the present disclosure, the compound represented by any one of formulas 1 to 18 may be comprised as a host material in a light-emitting layer, and the compound represented by any one of formulas 5 and 12 to 18 may be comprised as an electron transport material in an electron transport layer.

The present disclosure provides an organic electroluminescent compound represented by the following formula 5.

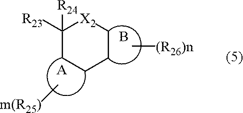

In formula 5,
X₂ represents -N=, —NR₀—, -O- or -S-;
R₀, R₂₃ and R₂₄, each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di- (3- to 30-membered)heteroarylamino, or a substituted or unsubstituted (C6-C30)aryl(3- to 30-membered)heteroarylamino;
A and B, each independently, represent a mono- or polycyclic, (C5-C20) alicyclic or aromatic ring, or the combination thereof, in which a carbon atom(s) of the ring may be replaced with at least one heteroatom selected from N, O, and S;
R₂₅ and R₂₆, each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di- (3- to 30-membered)heteroarylamino, a substituted or unsubstituted (C6-C30)aryl(3- to 30-membered)heteroarylamino, or —(L₁)ₐ—Ar₁;
L₁, each independently, represents a single bond, a substituted or unsubstituted (C6-C30)arylene, or a substituted or unsubstituted (3- to 30-membered)heteroarylene;
Ar₁ represents a substituted or unsubstituted (C6-C30)aryl, a substituted or unsubstituted (3- to 30-membered)heteroaryl, or

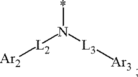

L₂ and L₃, each independently, represent a single bond, a substituted or unsubstituted (C6-C30)arylene, or a substituted or unsubstituted (3- to 30-membered)heteroarylene;
Ar₂ and Ar₃, each independently, represent a substituted or unsubstituted (C6-C30)aryl, a substituted or unsubstituted (3- to 30-membered)heteroaryl, or a substituted or unsubstituted (C3-C30)cycloalkyl;
m and n. each independently, represent an integer of 1 to 4, where if m and n are an integer of 2 or more, each of R₂₅ and each of R₂₆ may be the same or different; and
a represents an integer of 1 or 2, where if a is an integer of 2, each of L₁ may be the same or different.

In formula 5, X₂ represents, for example, -O-.

In formula 5, R₂₃ and R₂₄, each independently, may be, for example, hydrogen, deuterium, a methyl unsubstituted or substituted with deuterium, or a phenyl, etc.

In formula 5, according to one embodiment of the present disclosure, A and B, each independently, represent a mono- or polycyclic, (C6-C10) aromatic ring, in which a carbon atom(s) of the ring may be replaced with at least one heteroatom selected from N, O, and S. For example, A and B, each independently, may be a benzene ring or a naphthalene ring.

In formula 5, according to one embodiment of the present disclosure, at least one of R₂₅(s) and R₂₆(s) represents —(L₁)ₐ—Ar₁. For example, any one of R₂₅(s) and R₂₆(s) may be —(L₁)ₐ—Ar₁, and the others may be hydrogen.

The preferred embodiment of L₁ to L₃, Ar₁, to Ar₃, and a are as described in formula 1 above.

According to one embodiment of the present disclosure, formula 5 may be represented by any one of the following formulas 12 to 18.

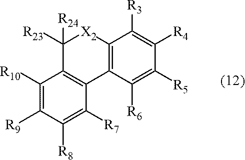

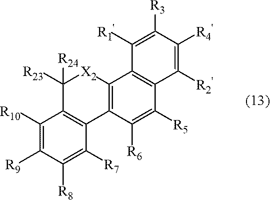

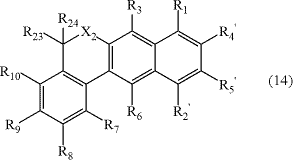

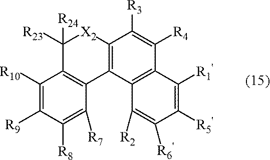

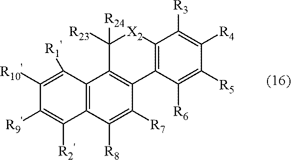

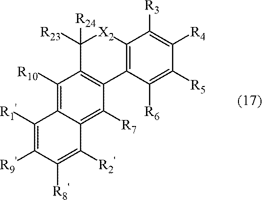

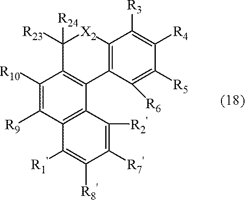

In formulas 12 to 18, R₃ to R₁₀, and R_{1′} to R_{10′}, each independently, are as defined for R₂₅ and R₂₆ in formula 5, and X₂, R₂₃, R₂₄, L₁, Ar₁, and a are as defined in formula 5. In any one of formulas 12 to 18, at least one, preferably any one of R₃ to R₁₀. and R_{1′} to R_{10′} represents —(L₁)ₐ—Ar₁.

The compounds represented by formulas 1 to 18 according to the present disclosure may be produced by synthetic methods known to one skilled in the art. For example, the compounds represented by formulas 1 and 5 to 18 of the present disclosure may be produced by referring to the following reaction scheme 1, and the compounds represented by formulas 2 to 4 of the present disclosure may be produced by referring to Korean Patent Application Laid-Open Nos. 10-2013-0130236 (published on Dec. 2, 2013), 10-2012-0102374 (published on Sep. 18, 2012), 10-2011-0015836 (published on Feb. 17, 2011), 10-2010-0108903 (published on Oct. 8, 2010), and 10-2014-0049531 (published on Apr. 25, 2014), etc., but is not limited thereto.

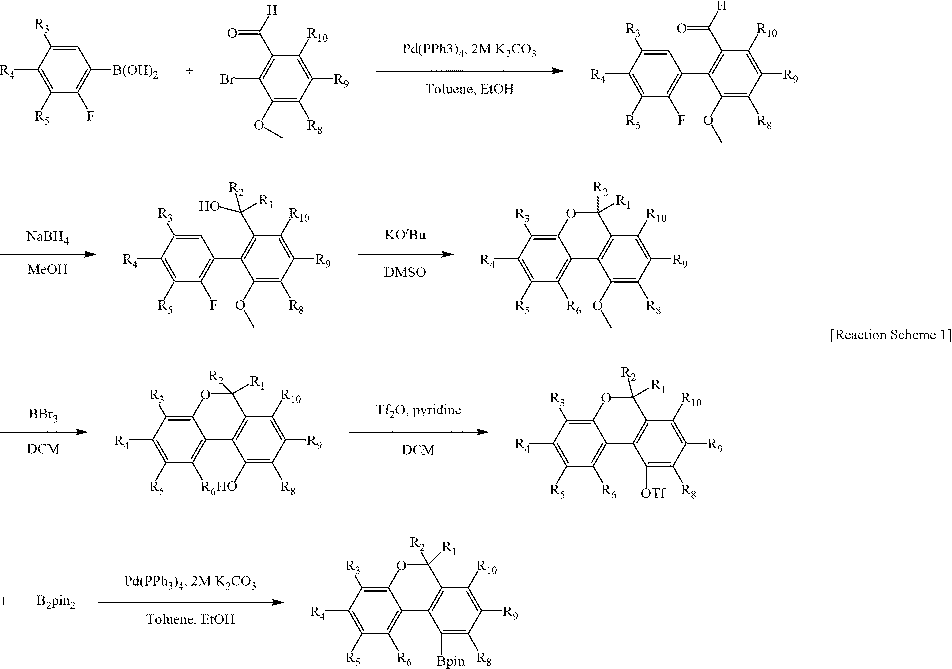

In reaction scheme 1, R₁ to R₁₀ are as defined in formula 1.

The present disclosure provides an organic electroluminescent device comprising an anode, a cathode, and at least one light-emitting layer between the anode and the cathode, wherein the at least one light-emitting layer comprises the plurality of host materials of the present disclosure. The first host material and the second host material of the present disclosure may be comprised in one light-emitting layer, or may be comprised in respective different light-emitting layers. The plurality of host materials of the present disclosure may comprise the compound represented by formula 1 and the compound represented by formula 2 in a ratio of about 1:99 to about 99:1, preferably in a ratio of about 10:90 to about 90:10, more preferably in a ratio of about 30:70 to about 70:30. Also, the compound represented by formula 1 and the compound represented by formula 2 in a desired ratio may be combined by mixing them in a shaker, by dissolving them in a glass tube by heat, or by dissolving them in a solvent, etc.

According to one embodiment of the present disclosure, the doping concentration of a dopant compound with respect to a host compound in the light-emitting layer is less than about 20 wt%. The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be a complex compound of a metal atom selected from iridium (Ir), osmium (Os), copper (Cu) and platinum (Pt), and preferably ortho-metallated complex compounds of a metal atom selected from iridium (Ir), osmium (Os), copper (Cu), and platinum (Pt), and more preferably ortho-metallated iridium complex compounds.

The dopant comprised in the organic electroluminescent device of the present disclosure may comprise a compound represented by the following formula 101, but is not limited thereto.

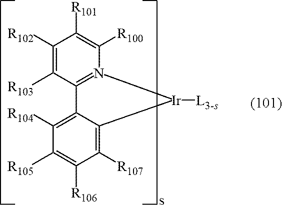

In formula 101,
L is selected from the following structures 1 to 3:

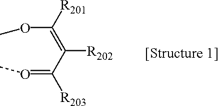

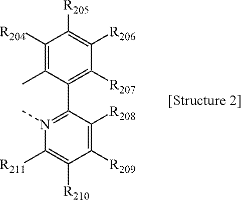

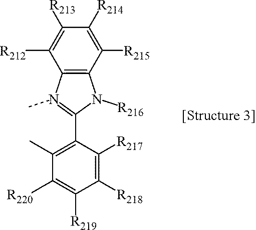

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a substituted or unsubstituted ring(s), e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a substituted or unsubstituted ring(s); and
s represents an integer of 1 to 3.

The specific examples of the dopant compound are as follows, but are not limited thereto.

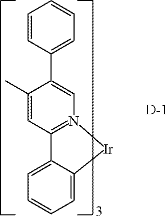

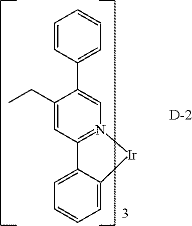

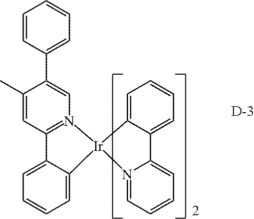

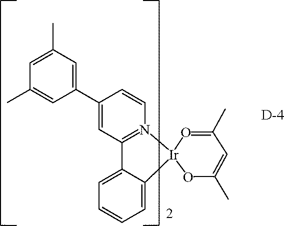

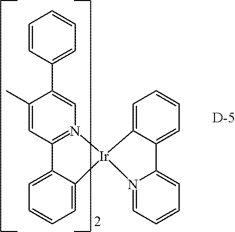

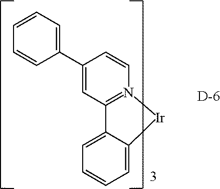

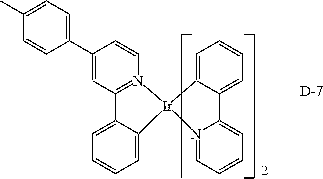

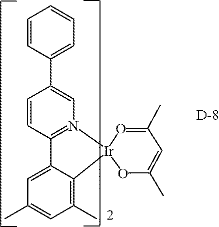

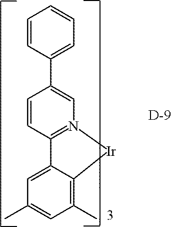

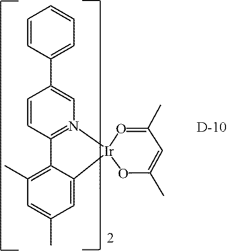

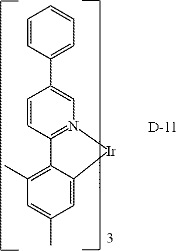

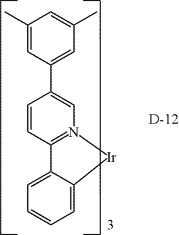

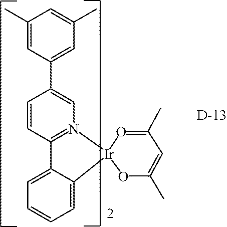

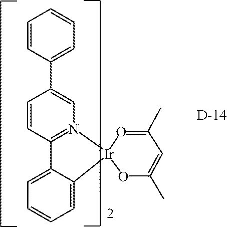

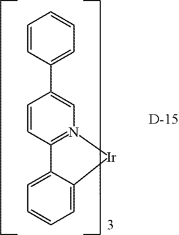

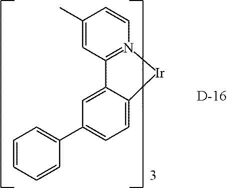

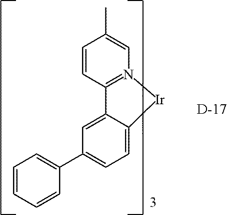

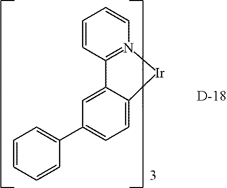

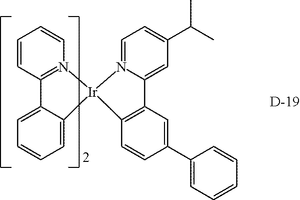

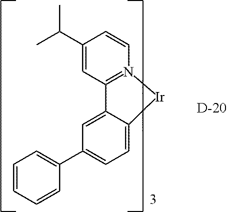

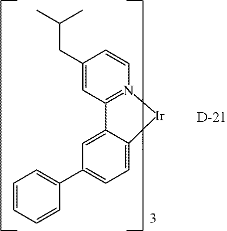

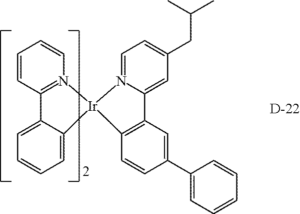

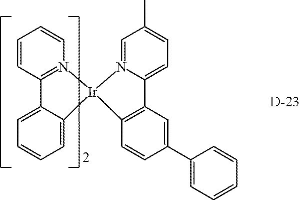

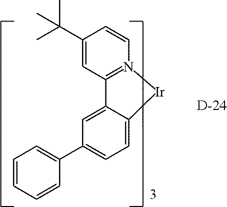

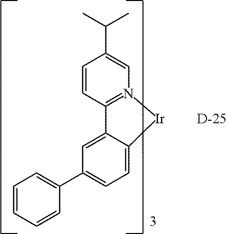

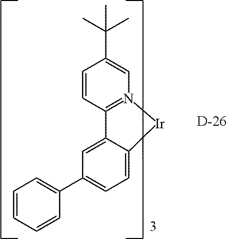

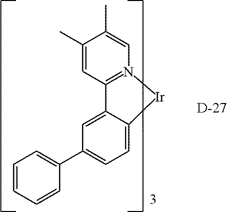

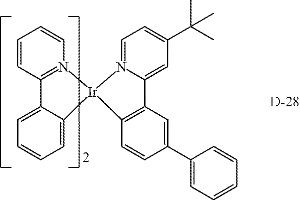

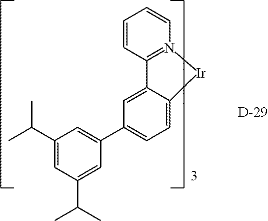

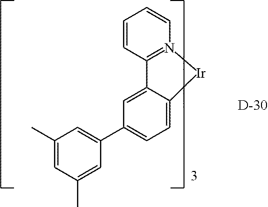

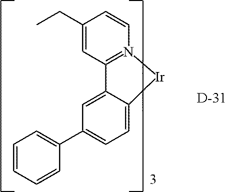

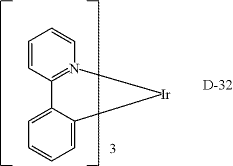

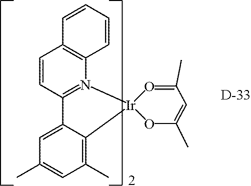

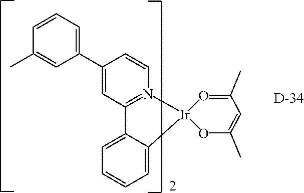

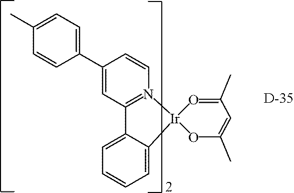

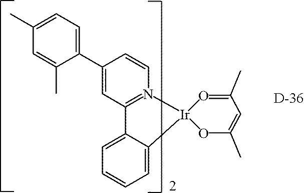

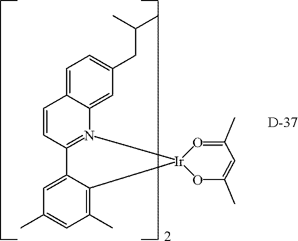

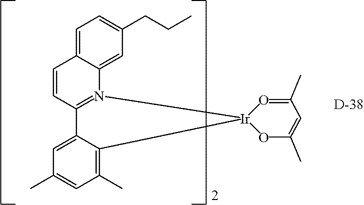

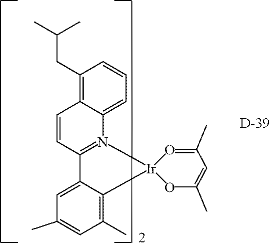

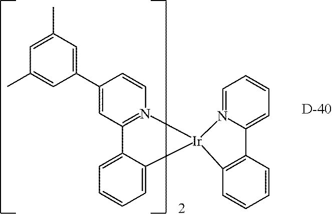

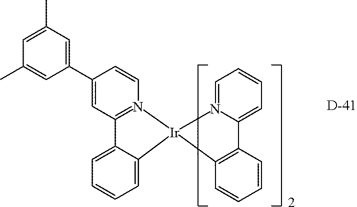

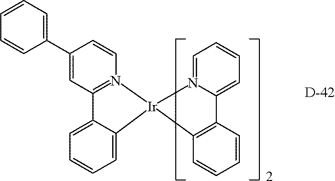

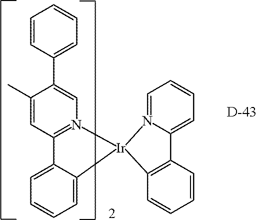

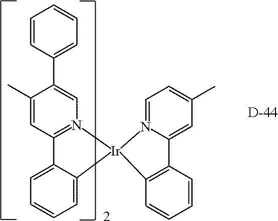

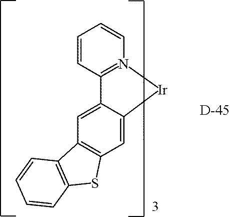

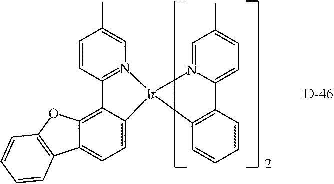

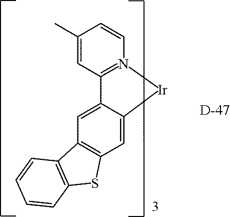

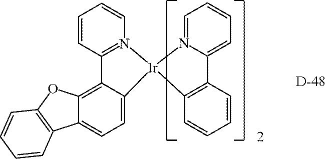

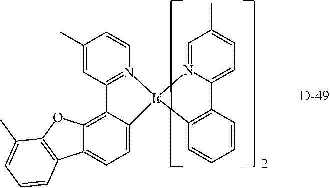

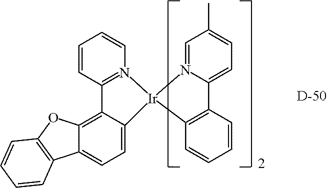

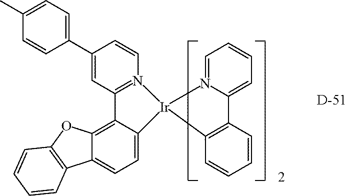

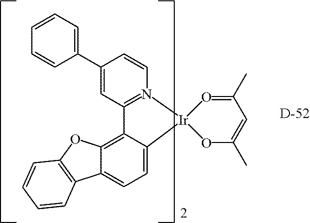

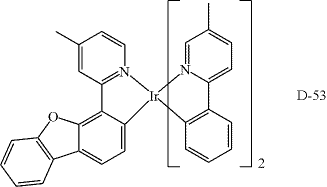

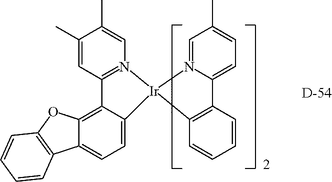

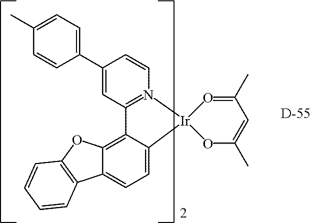

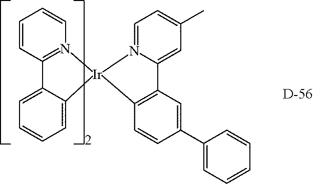

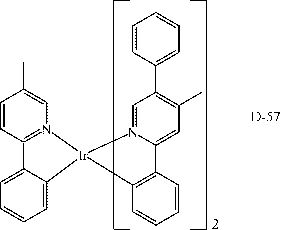

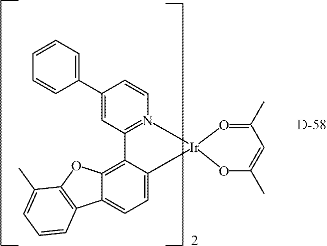

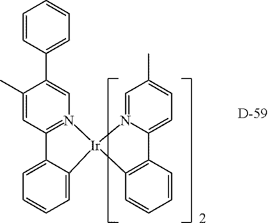

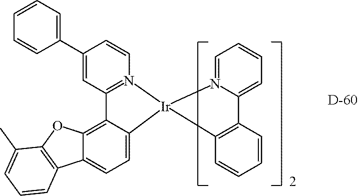

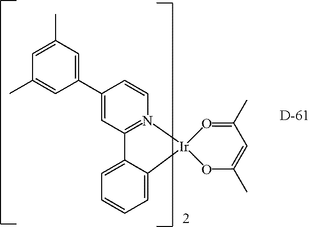

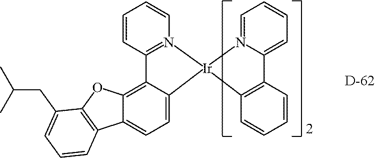

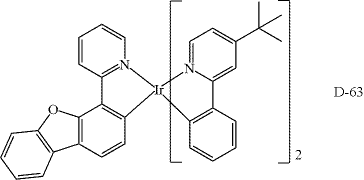

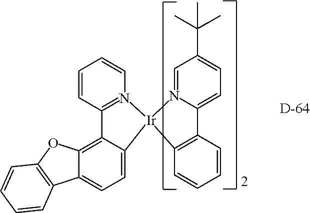

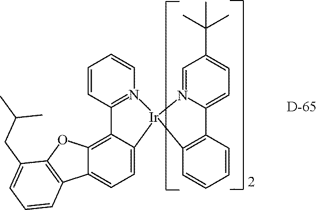

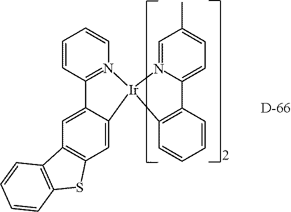

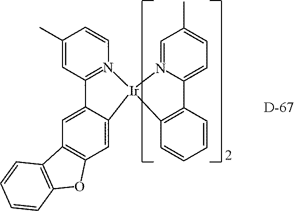

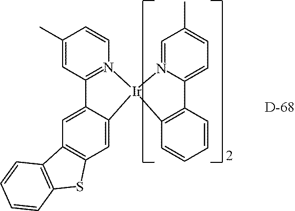

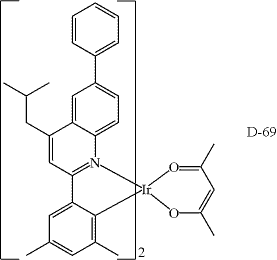

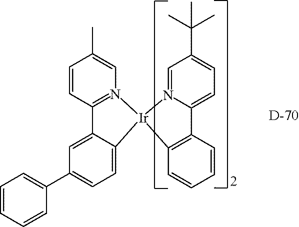

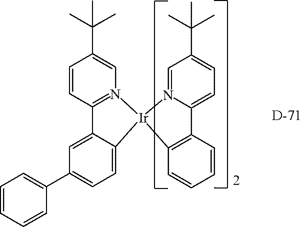

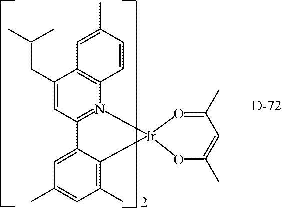

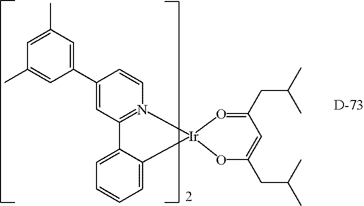

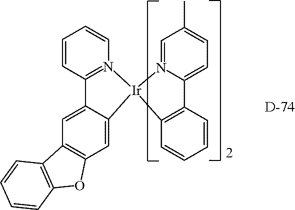

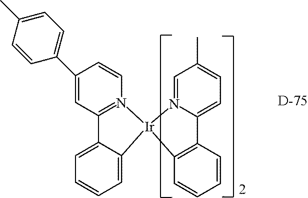

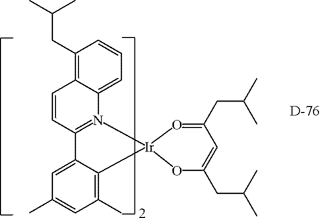

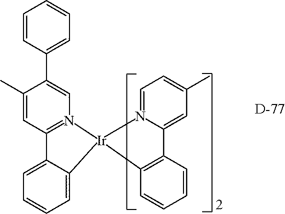

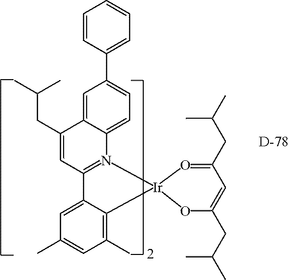

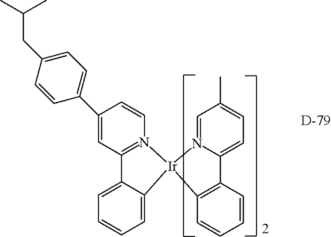

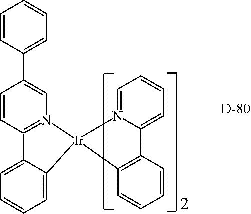

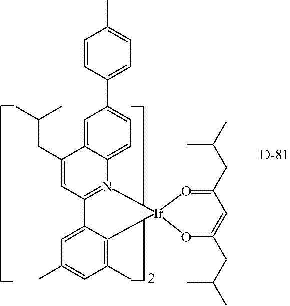

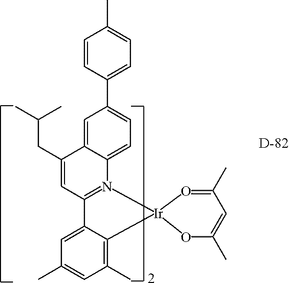

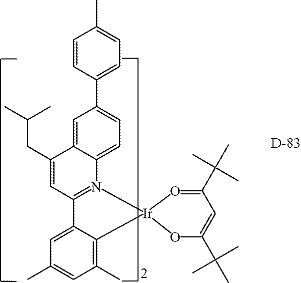

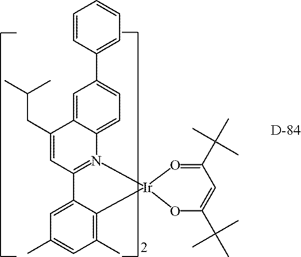

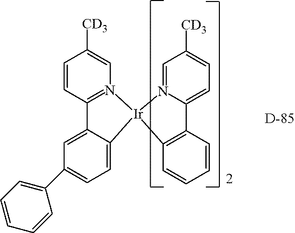

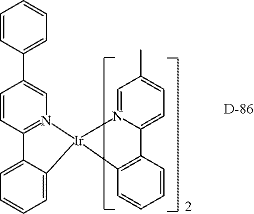

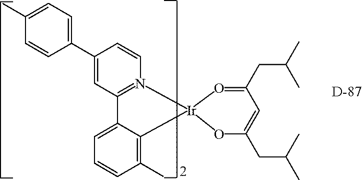

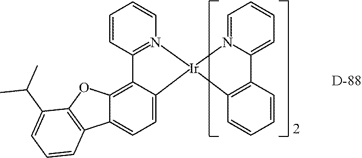

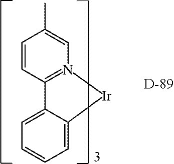

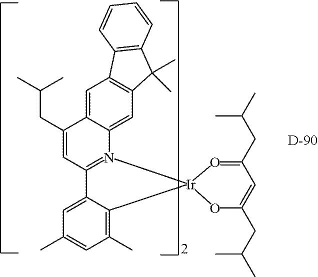

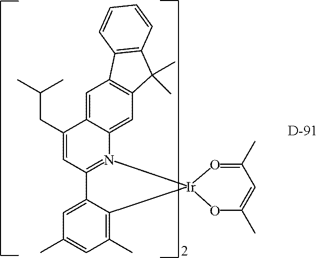

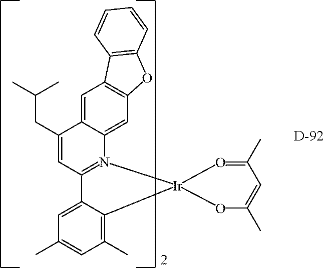

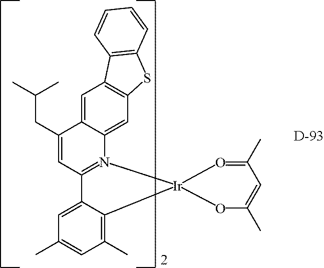

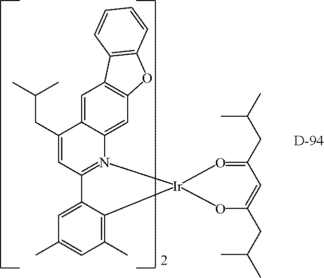

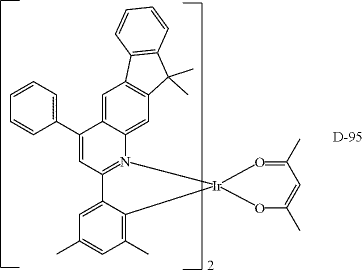

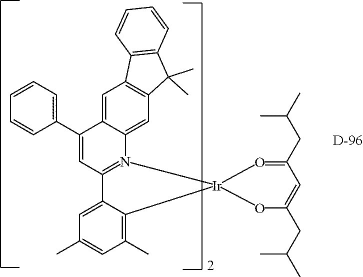

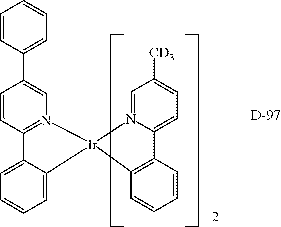

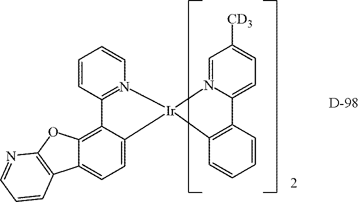

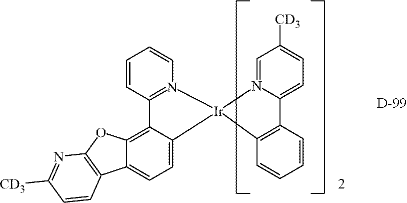

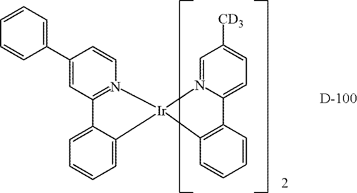

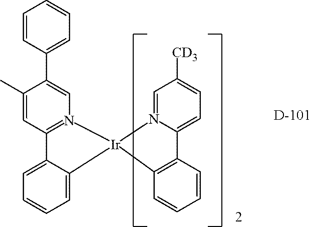

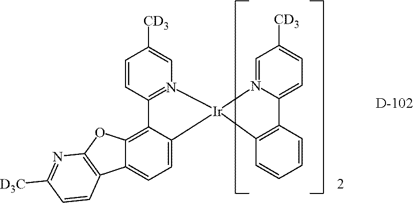

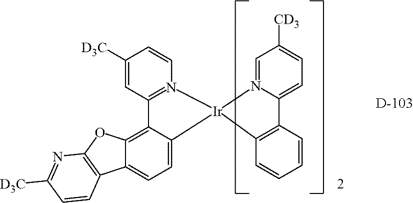

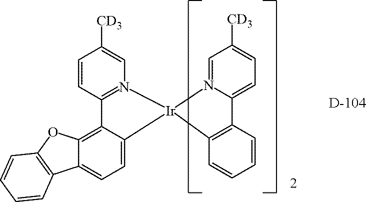

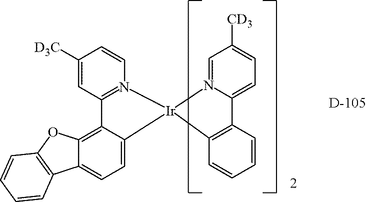

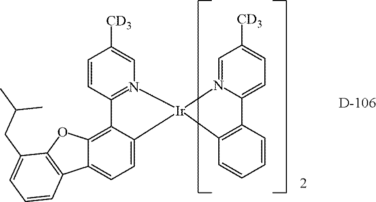

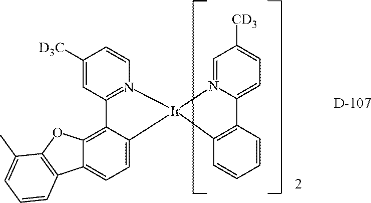

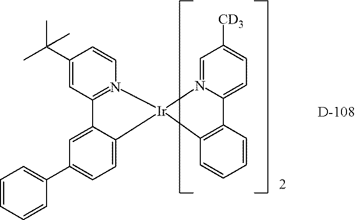

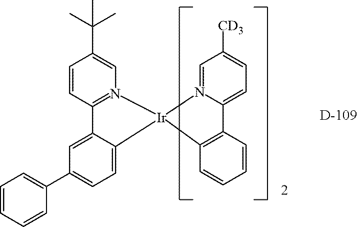

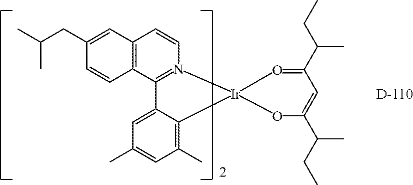

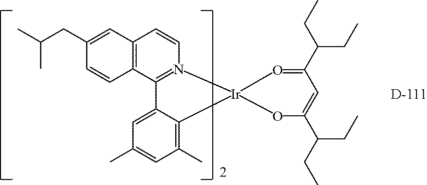

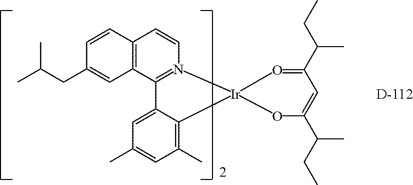

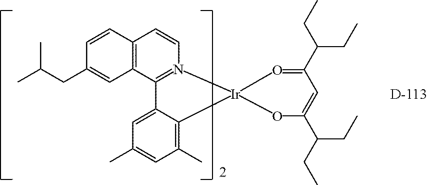

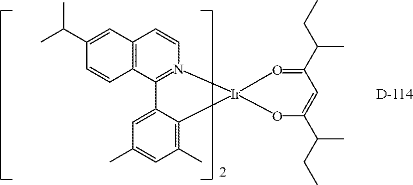

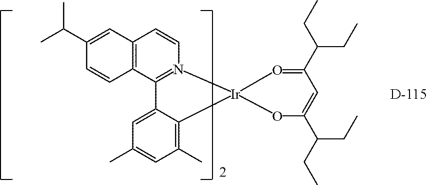

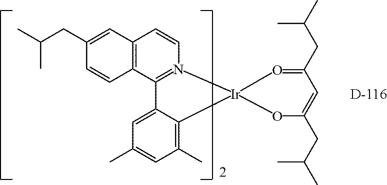

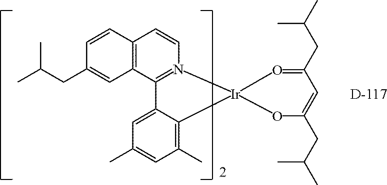

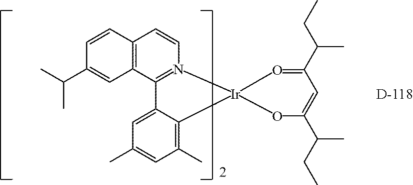

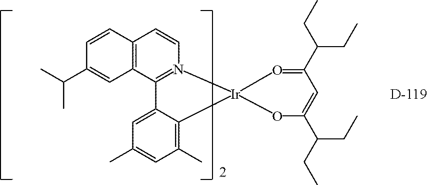

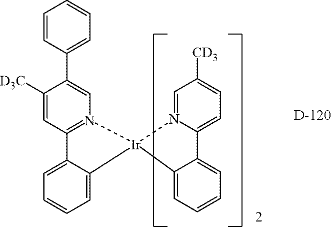

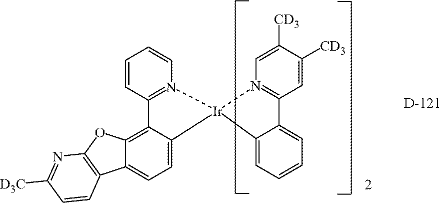

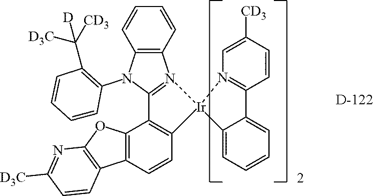

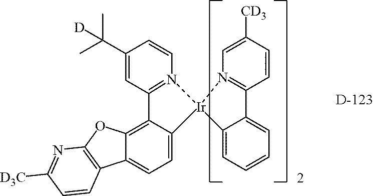

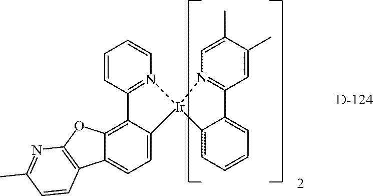

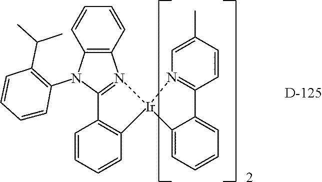

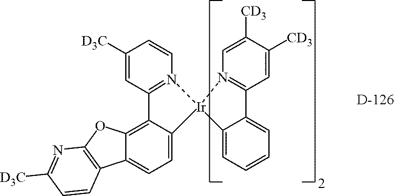

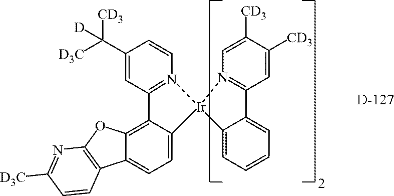

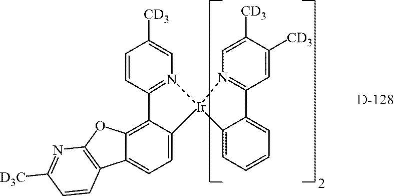

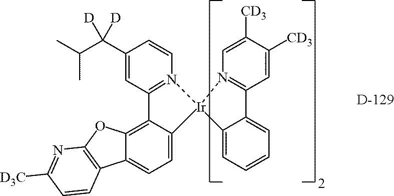

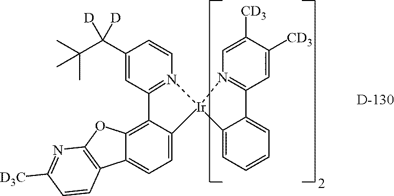

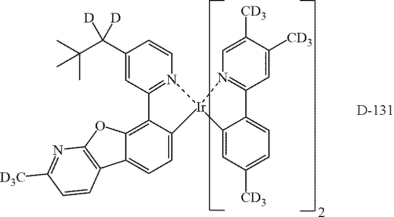

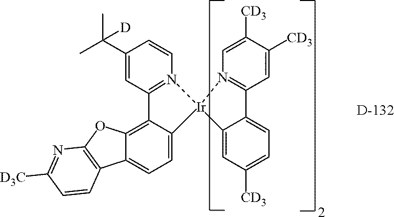

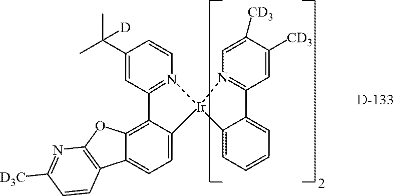

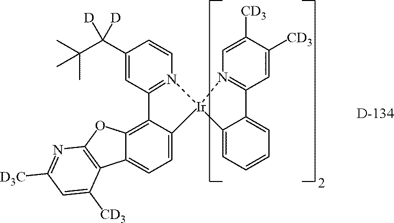

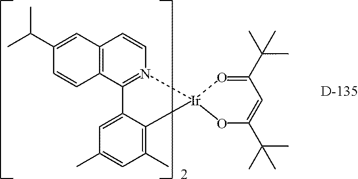

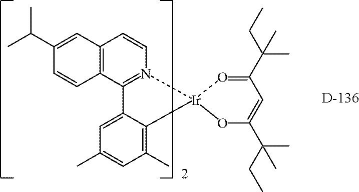

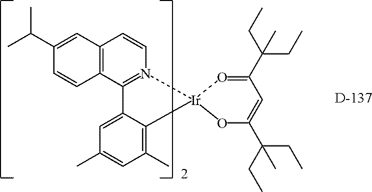

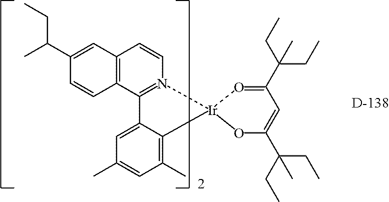

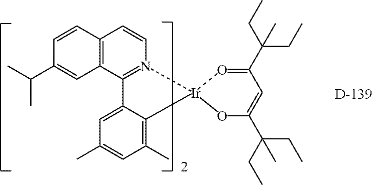

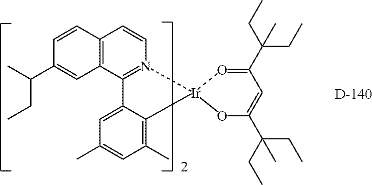

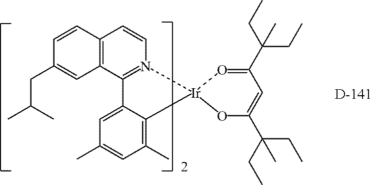

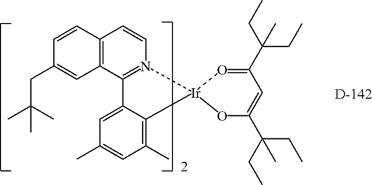

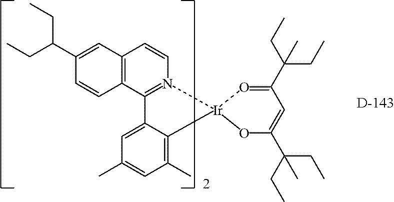

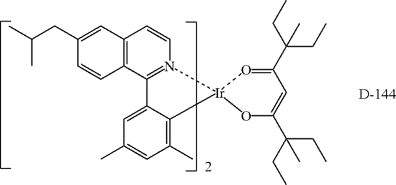

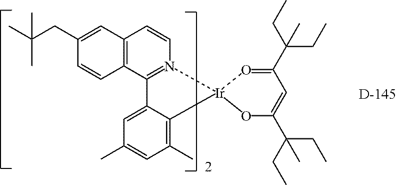

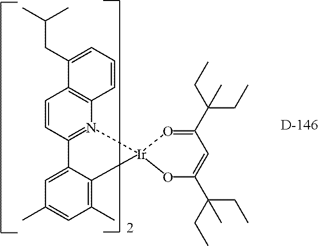

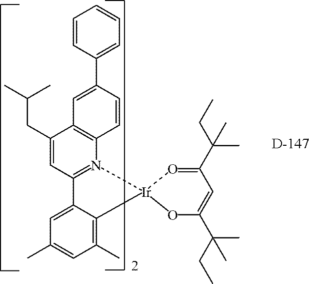

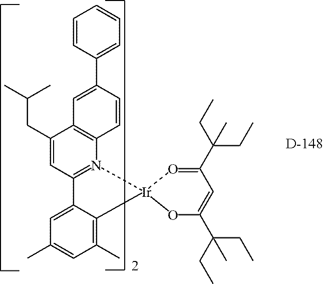

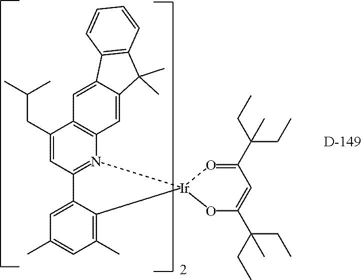

The organic electroluminescent device according to the present disclosure has an anode, a cathode, and at least one organic layer between the anode and the cathode. The organic layer may comprise a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant(s), and the electron injection layer may be further doped with an n-dopant(s).

The organic layer may further comprise at least one compound selected from the group consisting of arylamine-based compounds and styrylarylamine-based compounds.

Further, in the organic electroluminescent device of the present disclosure, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiOₓ(1≤X≤2), AlOₓ(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

The organic electroluminescent compound or the organic electroluminescent material according to one embodiment of the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The organic electroluminescent material according to one embodiment of the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When the first and second host compounds of the present disclosure are used to form a film, a coevaporation process or a mixture-evaporation process is carried out.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof, and luminous efficiency and lifetime properties of an organic electroluminescent device (OLED) comprising a plurality of host materials according to the present disclosure will be explained in detail with reference to the representative compounds of the present disclosure. The following examples only describe the properties of the OLED comprising the compound according to the present disclosure, but the present disclosure is not limited to the following examples.

### EXAMPLE 1: PREPARATION OF COMPOUND C-29

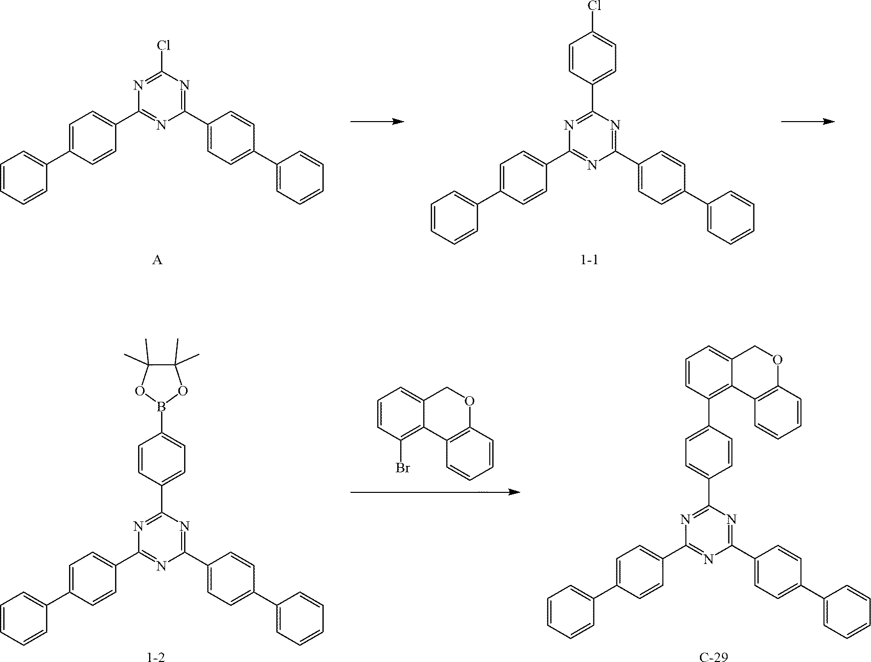

### Synthesis of Compound 1-1

Compound A (20.0 g, 47.60 mmol), 4-chlorophenylboronic acid (8.9 g, 57.10 mmol), tetrakis(triphenylphosphine)palladium(0) (11.0 g, 9.52 mmol), cesium carbonate (38.7 g, 119.00 mmol), 240 mL of toluene, 60 mL of ethanol, and 60 mL of water were added to a reaction vessel, and refluxed for 4 hours. After completion of the reaction, the reaction mixture was cooled to room temperature, and poured into methanol to precipitate a solid. The produced solid was dried, and purified by column chromatography to obtain compound 1-1 (21.1 g, yield: 90%).

### Synthesis of Compound 1-2

Compound 1-1 (8.0 g, 16.13 mmol), bis(pinacolato)diboron (4.9 g. 19.35 mmol), tris(dibenzylideneacetone)dipalladium(0) (0.74 g, 0.81 mmol), tricyclohexylphosphine (0.45 g, 1.61 mmol), potassium acetate (4.8 g, 48.4 mmol), and 80 mL of 1,4-dioxane were added to a reaction vessel, and refluxed for 12 hours. After completion of the reaction, the reaction mixture was cooled to room temperature, and filtered with diatomite. The solvent was removed from the collected filtrate by rotary evaporator. Thereafter, the residue was purified by column chromatography to obtain compound 1-2 (5.6 g, yield: 62%).

### Synthesis of Compound C-29

Compound 1-2 (5.6 g, 9.53 mmol), 10-bromo-6H-benzo[c]chromene (2.3 g, 8.92 mmol), tetrakis(triphenylphosphine)palladium(0) (0.62 g. 0.54 mmol), potassium carbonate (3.1 g, 22.28 mmol), 40 mL of toluene. 10 mL of ethanol, and 10 mL of water were added to a reaction vessel, and refluxed for 12 hours. After completion of the reaction, the reaction mixture was cooled to room temperature, and poured into methanol to precipitate a solid. The produced solid was dried, and purified by column chromatography to obtain compound C-29 (3.1 g, yield: 54%).

| | MW | M.P. |
|---|---|---|
| C-29 | 641.77 | 221° C. |

### EXAMPLE 2: PREPARATION OF COMPOUND C-40

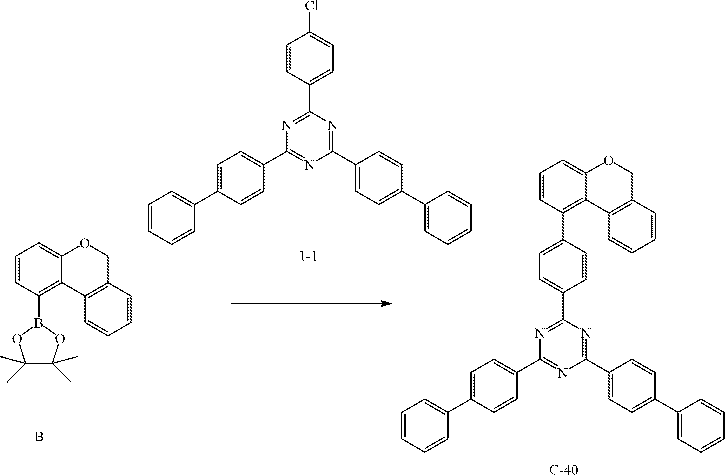

Compound B (4.8 g, 15.60 mmol), compound 1-1 (7.7 g, 15.60 mmol), palladium(II) acetate (0.18 g, 0.78 mmol), 2-dicyclohexylphosphino-2′,6′-dimethoxybiphenyl (0.64 g, 1.56 mmol), cesium carbonate (15.2 g, 46.80 mmol), 80 mL of toluene, 20 mL of ethanol, and 20 mL of water were added to a reaction vessel, and refluxed for 1 hour. After completion of the reaction, the reaction mixture was cooled to room temperature, and poured into methanol to precipitate a solid. The produced solid was filtered and dried. Thereafter, the dried solid was purified by column chromatography to obtain compound C-40 (3.4 g, yield: 34%).

| | MW | M.P. |
|---|---|---|
| C-40 | 641.77 | 206° C. |

### EXAMPLE 3: PREPARATION OF COMPOUND C-1

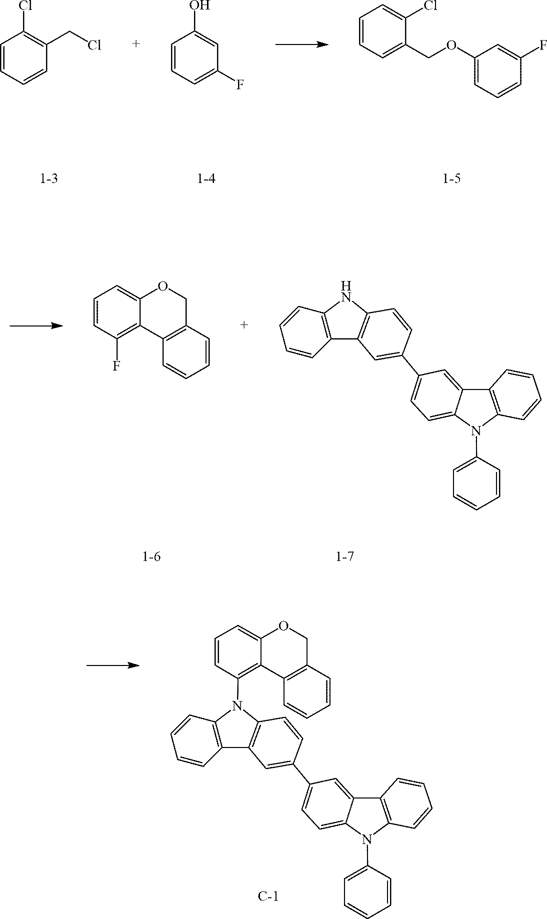

### Synthesis of Compound 1-5

Compound 1-3 (21 g, 130.4 mmol), compound 1-4 (24 g, 260.8 mmol), K₂CO₃ (54.1 g, 391.2 mmol), Nal (1.95 g, 13.04 mmol) and acetone (150 mL) were added to a reaction vessel, and stirred under reflux for 24 hours under nitrogen atmosphere. After completion of the reaction, the reaction mixture was cooled to room temperature, extracted with EtO₂, and dried with MgSO₄. The reaction mixture was concentrated, and purified by column chromatography to obtain compound 1-5 (30.9 g, yield: 99%).

### Synthesis of Compound 1-6

Compound 1-5 (30.9 g, 130.6 mmol), K₂CO₃ (36.1 g, 261.2 mmol), Pd(OAc)₂ (0.88 g, 3.92 mmol), PCy₃-HBF₄ (2.9 g, 7.84 mmol), and N,N-dimethylacetamide (40 mL) were added to a reaction vessel, and stirred under reflux for 24 hours under nitrogen atmosphere. After completion of the reaction, the reaction mixture was cooled to room temperature, concentrated, and then purified by column chromatography to obtain compound 1-6 (26 g, yield: 99%).

### Synthesis of Compound C-1

Compound 1-6 (26 g, 129.86 mmol), compound 1-7 (53 g, 129.86 mmol), Cs₂CO₃ (126 g, 389.58 mmol), and N,N-dimethylformamide (DMF) (260 mL) were added to a reaction vessel, and stirred under reflux for 24 hours. After completion of the reaction, the reaction mixture was cooled to room temperature, extracted with dichloromethane (DCM), dried with MgSO₄, and concentrated in a concentrator. The reaction mixture and 84 mL of toluene were added to a reaction vessel, refluxed for 1 hour, cooled to room temperature, and recrystallized. Thereafter, the resulting product was purified by column chromatography to obtain compound C-1 (14 g. yield: 18%).

| | MW | M.P. |
|---|---|---|
| C-1 | 588.71 | 267° C. |

### DEVICE EXAMPLE 1: PRODUCING A GREEN LIGHT-EMITTING OLED ACCORDING TO THE PRESENT DISCLOSURE

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt% based on the total amount of compound HI-1 and compound HT-1 to form a hole injection layer having a thickness of 10 nm. Next, compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: the first host compound (compound C-1) and the second host compound (compound H2-125) shown in Table 1 below were introduced into two cells of the vacuum vapor deposition apparatus as hosts, and compound D-130 was introduced into another cell as a dopant. The two host materials were evaporated at different rates of 2:1 and the dopant material was simultaneously evaporated at a different rate, and the dopant was deposited in a doping amount of 10 wt% based on the total amount of the hosts and the dopant to form a light-emitting layer having a thickness of 40 nm on the second hole transport layer. Next, compound ET-1 and compound EI-1 as electron transport materials were deposited in a weight ratio of 40:60 to form an electron transport layer having a thickness of 35 nm on the light-emitting layer. After depositing compound EI-1 as an electron injection layer having a thickness of 2 nm on the electron transport layer, an AI cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### COMPARATIVE EXAMPLE 1: PRODUCING AN OLED COMPRISING A CONVENTIONAL COMPOUND AS A HOST

An OLED was produced in the same manner as in Device Example 1, except that compound H2-125 was used solely as the host of the light-emitting layer.

The driving voltage, luminous efficiency, and light-emitting color at a luminance of 1,000 nit of the OLEDs produced in Device Example 1 and Comparative Example 1 are provided in Table 1 below.

TABLE 1

| | First Host | Second Host | Driving Voltage [V] | Luminous Efficiency [cd/A] | Light-Emitting Color |
|---|---|---|---|---|---|
| Device Example 1 | C-1 | H2-125 | 3.2 | 104.9 | Green |
| Comparative Example 1 | - | H2-125 | 3.0 | 85.3 | Green |

From Table 1 above, it can be seen that the OLED comprising a specific combination of compounds according to the present disclosure as host materials exhibits equivalent or similar driving voltage and higher luminous efficiency, compared to the OLEDs comprising the conventional compound as a single host material.

### DEVICE EXAMPLE 2: PRODUCING A BLUE LIGHT-EMITTING OLED ACCORDING TO THE PRESENT DISCLOSURE

An OLED was produced in the same manner as in Device Example 1, except that the second hole transport layer, the light-emitting layer, the electron buffer layer, and the electron transport layer were deposited as follows: Compound HT-3 was introduced into another cell of the vacuum vapor deposition apparatus and was evaporated by applying an electric current to the cell, thereby forming a second hole transport layer having a thickness of 5 nm on the first hole transport layer. Compound BH was introduced into a cell of the vacuum vapor deposition apparatus as a host, and compound BD was introduced into another cell as a dopant. The host material and the dopant material were evaporated at different rates, and the dopant was deposited in a doping amount of 3 wt% based on the total amount of the host and the dopant to form a light-emitting layer having a thickness of 20 nm on the second hole transport layer. Next, compound A was deposited as an electron buffer layer having a thickness of 5 nm on the light-emitting layer. Thereafter, compound C-40 and compound El-1 as electron transport materials were deposited in a weight ratio of 50:50 to form an electron transport layer having a thickness of 30 nm on the electron buffer layer.

The driving voltage, luminous efficiency, and light-emitting color at a luminance of 1,000 nit, and the time taken for luminance to decrease from 100% to 95% at a luminance of 2,000 nit (lifetime; T95) of the OLED produced in Device Example 2 are provided in Table 2 below.

TABLE 2

| | Electron Transport Layer | Driving Voltage [V] | Luminous Efficiency [cd/A] | Light-Emitting Color | Lifetime (T95, hr) |
|---|---|---|---|---|---|
| Device Example 2 | C-40 | 3.3 | 7.4 | Blue | 15.4 |

From Table 2 above, it can be seen that the OLED comprising the compound according to the present disclosure as the electron transport material exhibits lower driving voltage, higher luminous efficiency, and significantly improved lifetime compared to the conventional OLED.

The compounds used in the Device Examples and the Comparative Example are shown in Table 3 below.

TABLE 3

| | |
|---|---|
| Hole Injection Layer/ Hole Transport Layer | 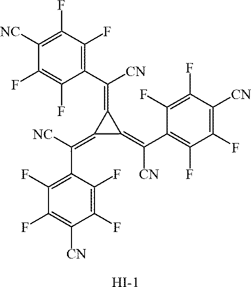 |
| | 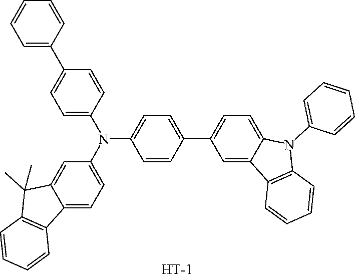 |
| | 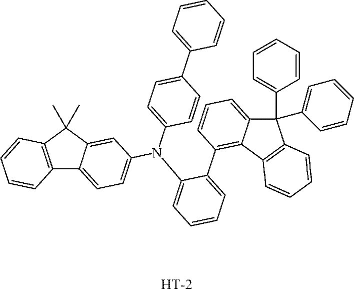 |
| | 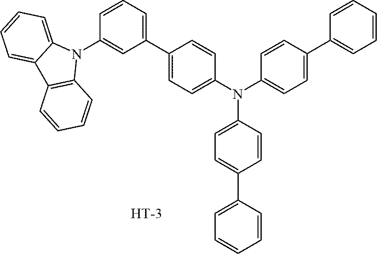 |
| Light-Emitting Layer/ Electron Buffer Layer | 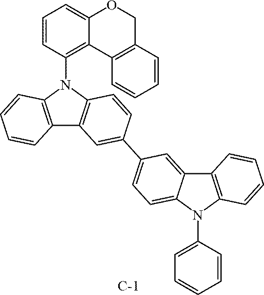 |
| | 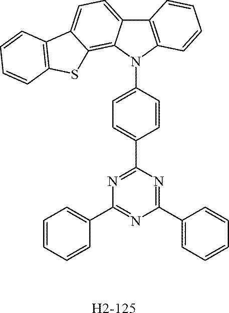 |
| | 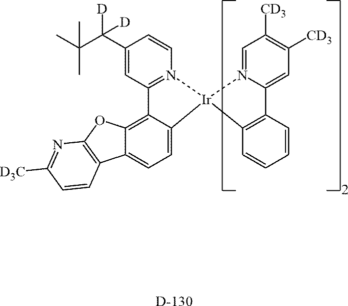 |
| | 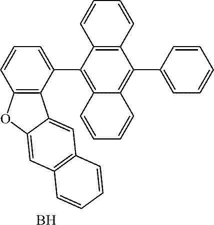 |
| | 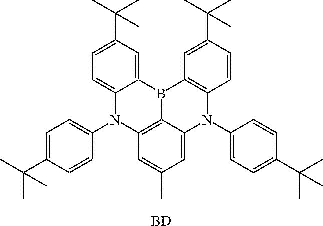 |
| | 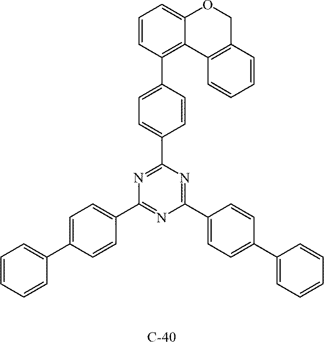 |
| | 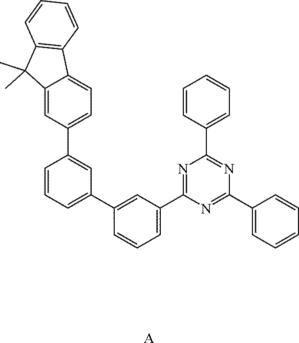 |
| Electron Transport Layer/ Electron Injection Layer | 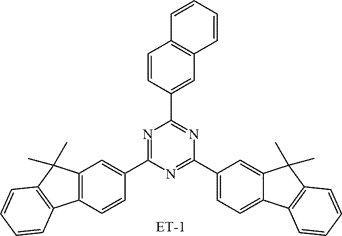 |
| | 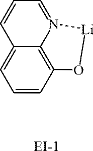 |

1. A plurality of host materials comprising a first host material comprising a compound represented by the following formula 1 and a second host material comprising a compound represented by the following formula 2: 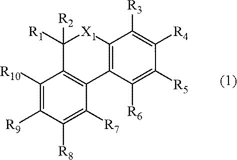
in formula 1,
X₁ represents -N=, —NR₀—, -O- or -S-;
R₀ represents a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di-(3- to 30-membered)heteroarylamino, or a substituted or unsubstituted (C6-C30)aryl(3- to 30-membered)heteroarylamino;
R₁ to R₁₀, each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di- (3- to 30-membered)heteroarylamino, or a substituted or unsubstituted (C6-C30)aryl(3- to 30-membered)heteroarylamino; or may be linked to an adjacent substituent(s) to form a ring(s);
with the proviso that at least one of R₃ to R₁₀ comprises —(L₁)ₐ—Ar₁;
Ar₁ represents a substituted or unsubstituted (C6-C30)aryl, a substituted or unsubstituted (3- to 30-membered)heteroaryl, or 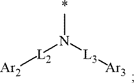
L₁ to L₃,each independently, represent a single bond, a substituted or unsubstituted (C6-C30)arylene, or a substituted or unsubstituted (3- to 30-membered)heteroarylene;
Ar₂ and Ar₃, each independently, represent a substituted or unsubstituted (C6-C30)aryl, a substituted or unsubstituted (3- to 30-membered)heteroaryl, or a substituted or unsubstituted (C3-C30)cycloalkyl; and
a represents an integer of 1 or 2, where if a is an integer of 2, each of L₁ may be the same or different; 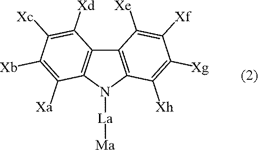
in formula 2,
Ma represents a substituted or unsubstituted (5- to 30-membered)heteroaryl containing a nitrogen(s);
La represents a single bond, a substituted or unsubstituted (C6-C30)arylene, or a substituted or unsubstituted (3- to 30-membered)heteroarylene;
Xa to Xh, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (3- to 30-membered)heteroarylamino, or a substituted or unsubstituted (C6-C30)aryl(3- to 30-membered)heteroarylamino; or may be linked to an adjacent substituent(s) to form any one ring selected from benzene, indole, indene, benzoindole, benzofuran, and benzothiophene, in which the ring may be further substituted with at least one of a (C1-C10)alkyl(s), and an (C6-C15)aryl(s) unsubstituted or substituted with a cyano(s) or a tri(C6-C18)arylsilyl(s); and
the heteroaryl comprises at least one heteroatom selected from B, N, O, S, Si and P.

2. The plurality of host materials according to claim 1, wherein the substituent(s) of the substituted alkyl, the substituted alkenyl, the substituted alkynyl, the substituted aryl, the substituted arylene, the substituted heteroaryl, the substituted heteroarylene, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di- alkylamino, the substituted mono- or di- alkenylamino, the substituted alkylalkenylamino, the substituted alkenylarylamino, the substituted alkenylheteroarylamino, the substituted alkylarylamino, the substituted mono- or di- arylamino, the substituted mono- or di- heteroarylamino, the substituted alkylheteroarylamino, the substituted arylheteroarylamino, and the substituted fused ring group of an aliphatic ring(s) and an aromatic ring(s), each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of deuterium, a halogen(s), a cyano(s), a (C1-C30)alkyl(s), a (C6-C30)aryl(s), a (3- to 30-membered)heteroaryl(s), and a tri(C6-C30)arylsilyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono- or di- (C2-C30)alkenylamino; a mono- or di- (C6-C30)arylamino; a mono- or di- (3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3-to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a (C6-C30)arylphosphine; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein Ma represents a substituted or unsubstituted pyridyl, a substituted or unsubstituted pyrimidinyl, a substituted or unsubstituted triazinyl, a substituted or unsubstituted pyrazinyl, a substituted or unsubstituted pyridazinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, or a substituted or unsubstituted benzothienopyrimidinyl.

4. The plurality of host materials according to claim 1, wherein formula 2 is represented by the following formula 3 or 4: 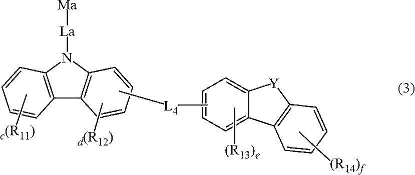 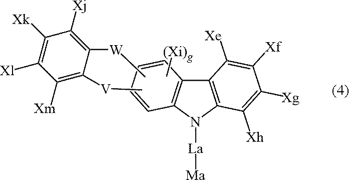
in formulas 3 and 4,
Y represents -S-, -O-, or —N(L₅—A₁)—;
L₄ and L₅, each independently, represent a single bond, or a substituted or unsubstituted (C6-C30)arylene;
A₁ represents a substituted or unsubstituted (C6-C30)aryl, or a substituted or unsubstituted (3- to 30-membered)heteroaryl;
R₁₁ and R₁₂, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (3- to 30-membered)heteroarylamino, or a substituted or unsubstituted (C6-C30)aryl(3- to 30-membered)heteroarylamino; R₁₃, R₁₄, and Xj to Xm, each independently, represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted fused ring group of a (C3-C30) aliphatic ring(s) and a (C6-C30) aromatic ring(s), -NR₁₈R₁₉, or -SiR₂₀R₂₁R₂₂; or may be linked to an adjacent substituent(s) to form a ring(s);
R₁₈ to R₂₂, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s); or may be linked to an adjacent substituent(s) to form a ring(s);
c and f, each independently, represent an integer of 1 to 4, and d and e, each independently, represent an integer of 1 to 3, where if c to f are an integer of 2 or more, each of R₁₁ to each of R₁₄ may be the same or different;
V and W, each independently, represent a single bond, NR₁₅, CR₁₆R₁₇, S, or O, with the proviso that both of V and W are not a single bond, or NR₁₅;
R₁₅ to R₁₇, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s);
Ma, La, and Xe to Xh are as defined in claim 1;
Xi, each independently, is as defined for Xa to Xd in claim 1; and
g represents an integer of 1 or 2, where if g is 2, each of Xi may be the same or different.

5. The plurality of host materials according to claim 1, wherein the compound represented by formula 1 is at least one selected from the group consisting of the following compounds: 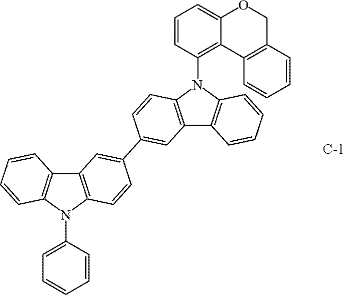 [SMILES: c1ccc(-n2c3ccccc3c3cc(-c4ccc5c(c4)c4ccccc4n5-c4cccc5c4-c4ccccc4CO5)ccc32)cc1] 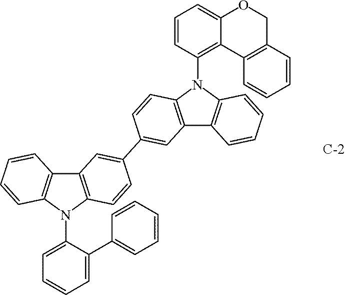 [SMILES: c1ccc(-c2ccccc2-n2c3ccccc3c3cc(-c4ccc5c(c4)c4ccccc4n5-c4cccc5c4-c4ccccc4CO5)ccc32)cc1] 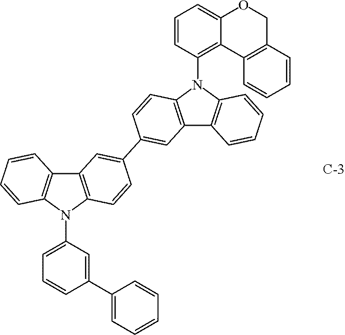 [SMILES: c1ccc(-c2cccc(-n3c4ccccc4c4cc(-c5ccc6c(c5)c5ccccc5n6-c5cccc6c5-c5ccccc5CO6)ccc43)c2)cc1] 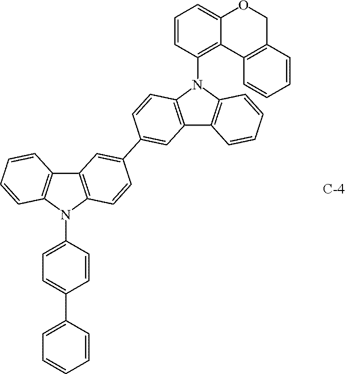 [SMILES: c1ccc(-c2ccc(-n3c4ccccc4c4cc(-c5ccc6c(c5)c5ccccc5n6-c5cccc6c5-c5ccccc5CO6)ccc43)cc2)cc1] 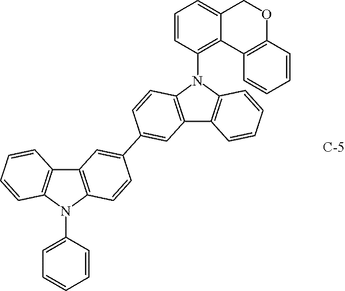 [SMILES: c1ccc(-n2c3ccccc3c3cc(-c4ccc5c(c4)c4ccccc4n5-c4cccc5c4-c4ccccc4OC5)ccc32)cc1] 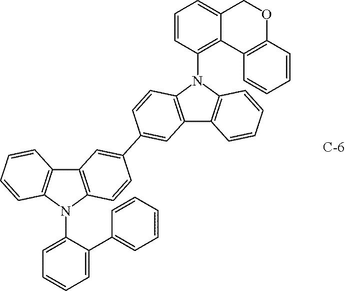 [SMILES: c1ccc(-c2ccccc2-n2c3ccccc3c3cc(-c4ccc5c(c4)c4ccccc4n5-c4cccc5c4-c4ccccc4OC5)ccc32)cc1] 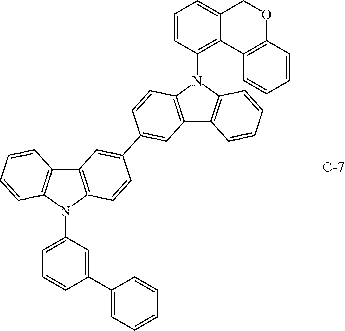 [SMILES: c1ccc(-c2cccc(-n3c4ccccc4c4cc(-c5ccc6c(c5)c5ccccc5n6-c5cccc6c5-c5ccccc5OC6)ccc43)c2)cc1] 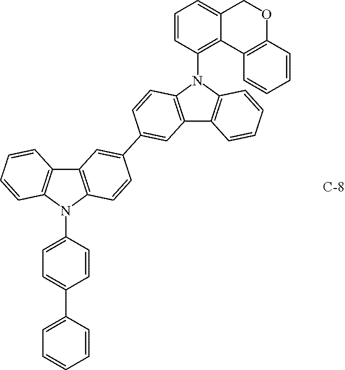 [SMILES: c1ccc(-c2ccc(-n3c4ccccc4c4cc(-c5ccc6c(c5)c5ccccc5n6-c5cccc6c5-c5ccccc5OC6)ccc43)cc2)cc1] 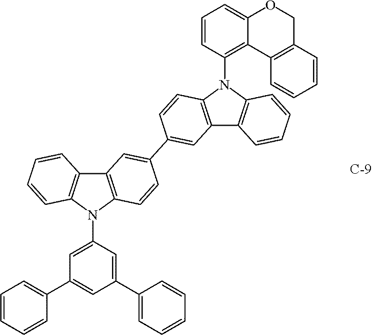 [SMILES: c1ccc(-c2cc(-c3ccccc3)cc(-n3c4ccccc4c4cc(-c5ccc6c(c5)c5ccccc5n6-c5cccc6c5-c5ccccc5CO6)ccc43)c2)cc1] 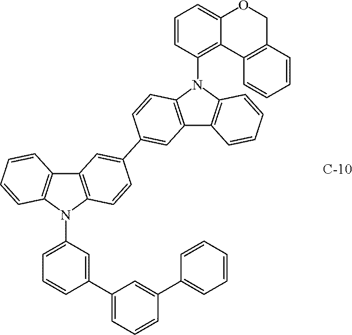 [SMILES: c1ccc(-c2cccc(-c3cccc(-n4c5ccccc5c5cc(-c6ccc7c(c6)c6ccccc6n7-c6cccc7c6-c6ccccc6CO7)ccc54)c3)c2)cc1] 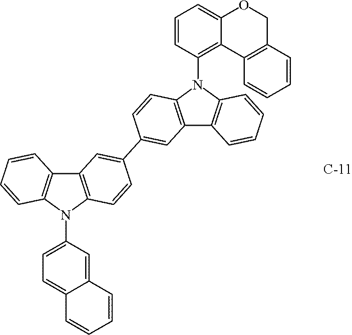 [SMILES: c1ccc2c(c1)COc1cccc(-n3c4ccccc4c4cc(-c5ccc6c(c5)c5ccccc5n6-c5ccc6ccccc6c5)ccc43)c1-2] 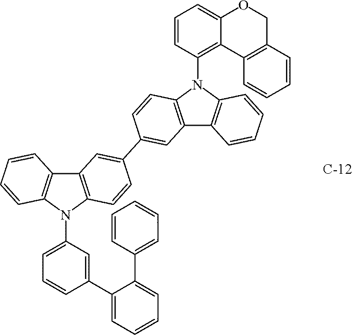 [SMILES: c1ccc(-c2ccccc2-c2cccc(-n3c4ccccc4c4cc(-c5ccc6c(c5)c5ccccc5n6-c5cccc6c5-c5ccccc5CO6)ccc43)c2)cc1] 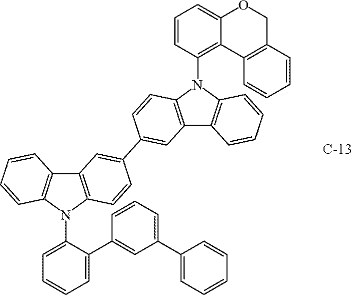 [SMILES: c1ccc(-c2cccc(-c3ccccc3-n3c4ccccc4c4cc(-c5ccc6c(c5)c5ccccc5n6-c5cccc6c5-c5ccccc5CO6)ccc43)c2)cc1] 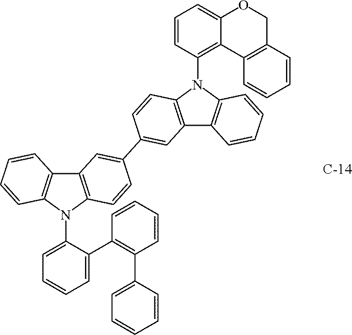 [SMILES: c1ccc(-c2ccccc2-c2ccccc2-n2c3ccccc3c3cc(-c4ccc5c(c4)c4ccccc4n5-c4cccc5c4-c4ccccc4CO5)ccc32)cc1] 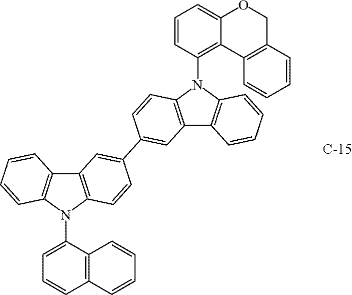 [SMILES: c1ccc2c(c1)COc1cccc(-n3c4ccccc4c4cc(-c5ccc6c(c5)c5ccccc5n6-c5cccc6ccccc56)ccc43)c1-2] 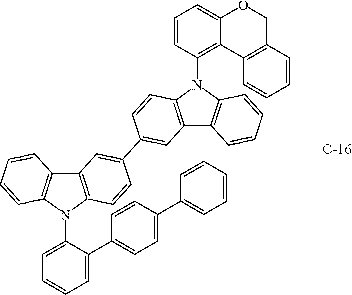 [SMILES: c1ccc(-c2ccc(-c3ccccc3-n3c4ccccc4c4cc(-c5ccc6c(c5)c5ccccc5n6-c5cccc6c5-c5ccccc5CO6)ccc43)cc2)cc1] 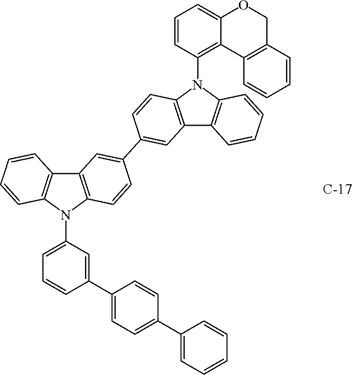 [SMILES: c1ccc(-c2ccc(-c3cccc(-n4c5ccccc5c5cc(-c6ccc7c(c6)c6ccccc6n7-c6cccc7c6-c6ccccc6CO7)ccc54)c3)cc2)cc1] 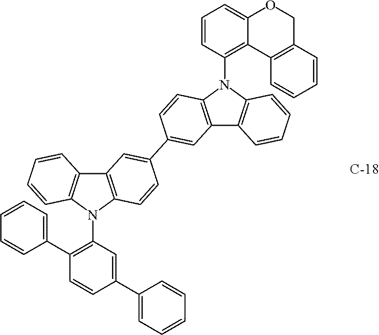 [SMILES: c1ccc(-c2ccc(-c3ccccc3)c(-n3c4ccccc4c4cc(-c5ccc6c(c5)c5ccccc5n6-c5cccc6c5-c5ccccc5CO6)ccc43)c2)cc1] 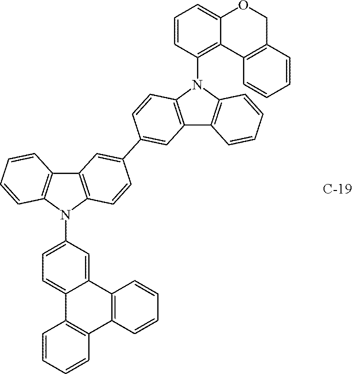 [SMILES: c1ccc2c(c1)COc1cccc(-n3c4ccccc4c4cc(-c5ccc6c(c5)c5ccccc5n6-c5ccc6c7ccccc7c7ccccc7c6c5)ccc43)c1-2] 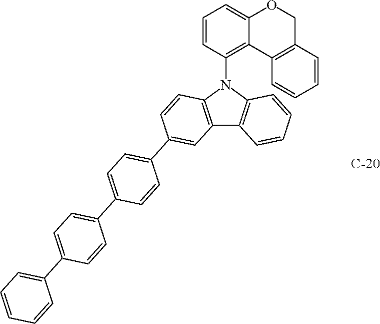 [SMILES: c1ccc(-c2ccc(-c3ccc(-c4ccc5c(c4)c4ccccc4n5-c4cccc5c4-c4ccccc4CO5)cc3)cc2)cc1] 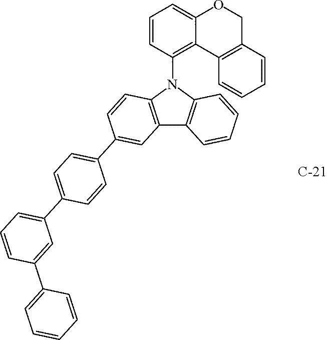 [SMILES: c1ccc(-c2cccc(-c3ccc(-c4ccc5c(c4)c4ccccc4n5-c4cccc5c4-c4ccccc4CO5)cc3)c2)cc1] 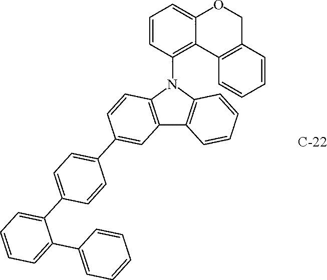 [SMILES: c1ccc(-c2ccccc2-c2ccc(-c3ccc4c(c3)c3ccccc3n4-c3cccc4c3-c3ccccc3CO4)cc2)cc1] 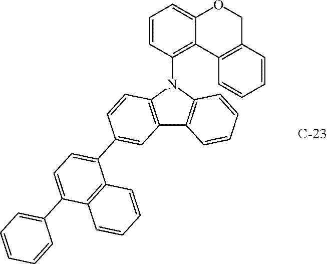 [SMILES: c1ccc(-c2ccc(-c3ccc4c(c3)c3ccccc3n4-c3cccc4c3-c3ccccc3CO4)c3ccccc23)cc1] 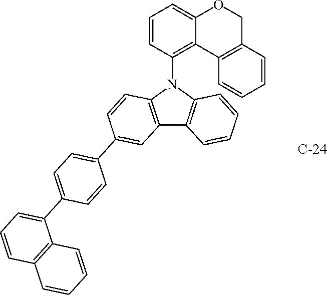 [SMILES: c1ccc2c(c1)COc1cccc(-n3c4ccccc4c4cc(-c5ccc(-c6cccc7ccccc67)cc5)ccc43)c1-2] 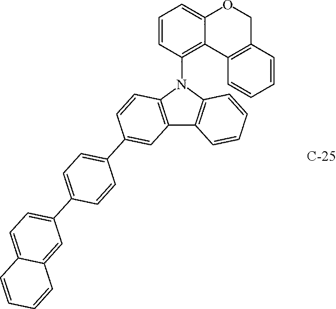 [SMILES: c1ccc2c(c1)COc1cccc(-n3c4ccccc4c4cc(-c5ccc(-c6ccc7ccccc7c6)cc5)ccc43)c1-2] 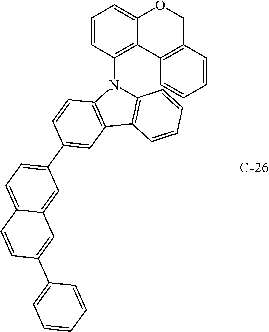 [SMILES: c1ccc(-c2ccc3ccc(-c4ccc5c(c4)c4ccccc4n5-c4cccc5c4-c4ccccc4CO5)cc3c2)cc1] 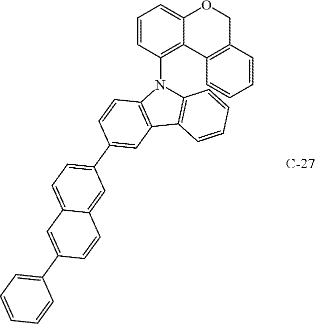 [SMILES: c1ccc(-c2ccc3cc(-c4ccc5c(c4)c4ccccc4n5-c4cccc5c4-c4ccccc4CO5)ccc3c2)cc1] 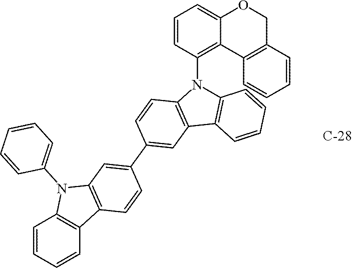 [SMILES: c1ccc(-n2c3ccccc3c3ccc(-c4ccc5c(c4)c4ccccc4n5-c4cccc5c4-c4ccccc4CO5)cc32)cc1] 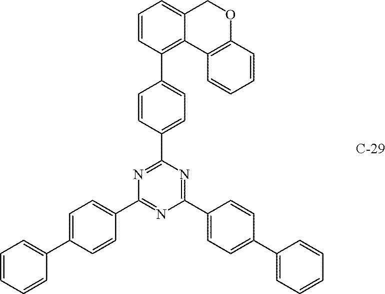 [SMILES: c1ccc(-c2ccc(-c3nc(-c4ccc(-c5ccccc5)cc4)nc(-c4ccc(-c5cccc6c5-c5ccccc5OC6)cc4)n3)cc2)cc1] 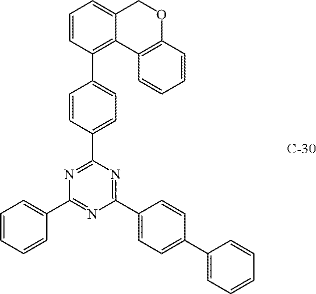 [SMILES: c1ccc(-c2ccc(-c3nc(-c4ccccc4)nc(-c4ccc(-c5cccc6c5-c5ccccc5OC6)cc4)n3)cc2)cc1] 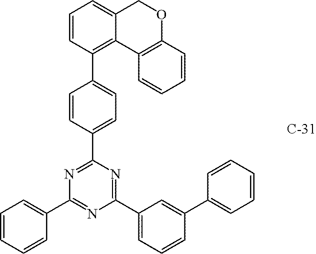 [SMILES: c1ccc(-c2cccc(-c3nc(-c4ccccc4)nc(-c4ccc(-c5cccc6c5-c5ccccc5OC6)cc4)n3)c2)cc1] 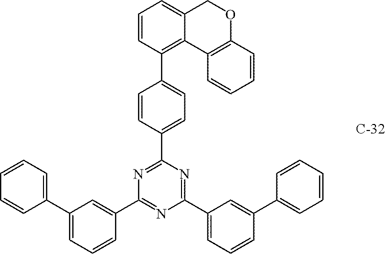 [SMILES: c1ccc(-c2cccc(-c3nc(-c4ccc(-c5cccc6c5-c5ccccc5OC6)cc4)nc(-c4cccc(-c5ccccc5)c4)n3)c2)cc1] 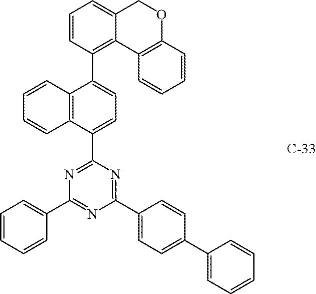 [SMILES: c1ccc(-c2ccc(-c3nc(-c4ccccc4)nc(-c4ccc(-c5cccc6c5-c5ccccc5OC6)c5ccccc45)n3)cc2)cc1] 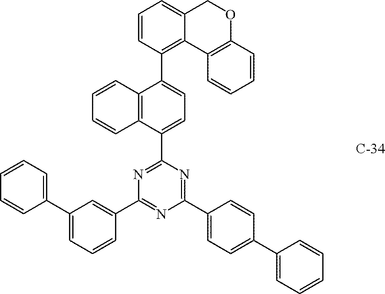 [SMILES: c1ccc(-c2ccc(-c3nc(-c4cccc(-c5ccccc5)c4)nc(-c4ccc(-c5cccc6c5-c5ccccc5OC6)c5ccccc45)n3)cc2)cc1] 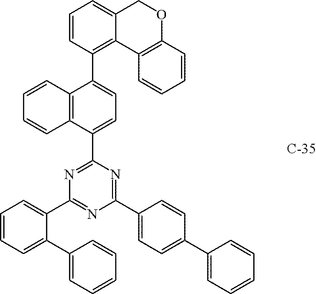 [SMILES: c1ccc(-c2ccc(-c3nc(-c4ccccc4-c4ccccc4)nc(-c4ccc(-c5cccc6c5-c5ccccc5OC6)c5ccccc45)n3)cc2)cc1] 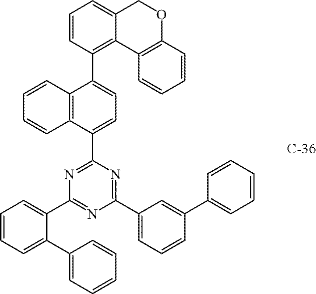 [SMILES: c1ccc(-c2cccc(-c3nc(-c4ccccc4-c4ccccc4)nc(-c4ccc(-c5cccc6c5-c5ccccc5OC6)c5ccccc45)n3)c2)cc1] 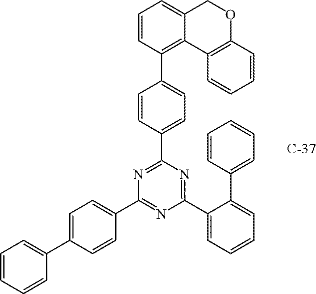 [SMILES: c1ccc(-c2ccc(-c3nc(-c4ccc(-c5cccc6c5-c5ccccc5OC6)cc4)nc(-c4ccccc4-c4ccccc4)n3)cc2)cc1] 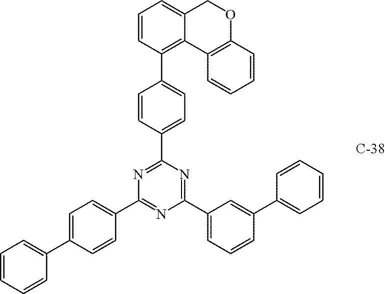 [SMILES: c1ccc(-c2ccc(-c3nc(-c4ccc(-c5cccc6c5-c5ccccc5OC6)cc4)nc(-c4cccc(-c5ccccc5)c4)n3)cc2)cc1] 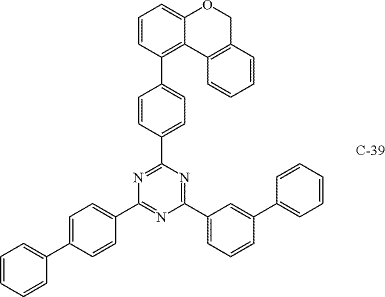 [SMILES: c1ccc(-c2ccc(-c3nc(-c4ccc(-c5cccc6c5-c5ccccc5CO6)cc4)nc(-c4cccc(-c5ccccc5)c4)n3)cc2)cc1] 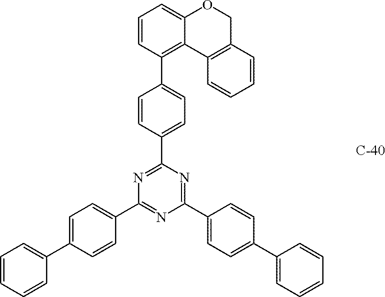 [SMILES: c1ccc(-c2ccc(-c3nc(-c4ccc(-c5ccccc5)cc4)nc(-c4ccc(-c5cccc6c5-c5ccccc5CO6)cc4)n3)cc2)cc1] 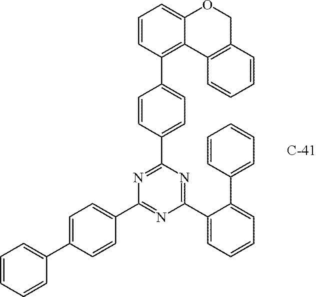 [SMILES: c1ccc(-c2ccc(-c3nc(-c4ccc(-c5cccc6c5-c5ccccc5CO6)cc4)nc(-c4ccccc4-c4ccccc4)n3)cc2)cc1] 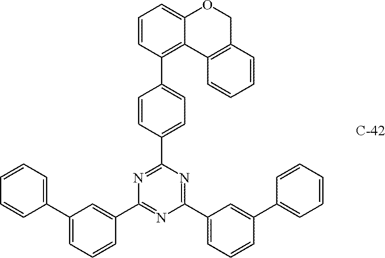 [SMILES: c1ccc(-c2cccc(-c3nc(-c4ccc(-c5cccc6c5-c5ccccc5CO6)cc4)nc(-c4cccc(-c5ccccc5)c4)n3)c2)cc1] 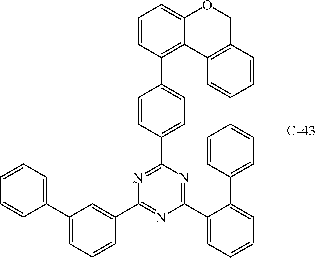 [SMILES: c1ccc(-c2cccc(-c3nc(-c4ccc(-c5cccc6c5-c5ccccc5CO6)cc4)nc(-c4ccccc4-c4ccccc4)n3)c2)cc1] 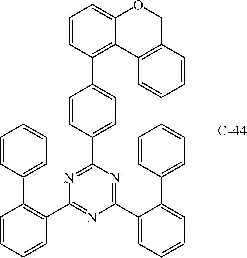 [SMILES: c1ccc(-c2ccccc2-c2nc(-c3ccc(-c4cccc5c4-c4ccccc4CO5)cc3)nc(-c3ccccc3-c3ccccc3)n2)cc1] 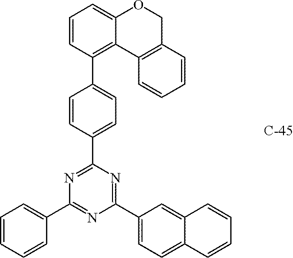 [SMILES: c1ccc(-c2nc(-c3ccc(-c4cccc5c4-c4ccccc4CO5)cc3)nc(-c3ccc4ccccc4c3)n2)cc1] 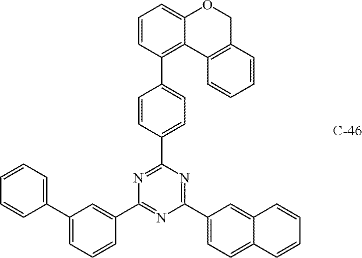 [SMILES: c1ccc(-c2cccc(-c3nc(-c4ccc(-c5cccc6c5-c5ccccc5CO6)cc4)nc(-c4ccc5ccccc5c4)n3)c2)cc1] 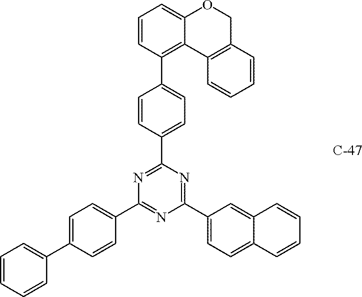 [SMILES: c1ccc(-c2ccc(-c3nc(-c4ccc(-c5cccc6c5-c5ccccc5CO6)cc4)nc(-c4ccc5ccccc5c4)n3)cc2)cc1] 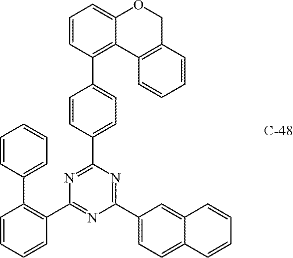 [SMILES: c1ccc(-c2ccccc2-c2nc(-c3ccc(-c4cccc5c4-c4ccccc4CO5)cc3)nc(-c3ccc4ccccc4c3)n2)cc1] 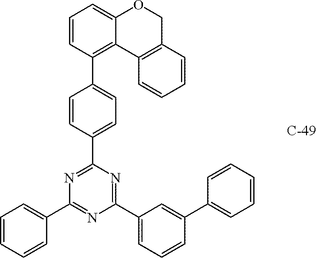 [SMILES: c1ccc(-c2cccc(-c3nc(-c4ccccc4)nc(-c4ccc(-c5cccc6c5-c5ccccc5CO6)cc4)n3)c2)cc1] 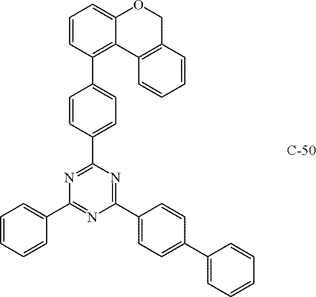 [SMILES: c1ccc(-c2ccc(-c3nc(-c4ccccc4)nc(-c4ccc(-c5cccc6c5-c5ccccc5CO6)cc4)n3)cc2)cc1] 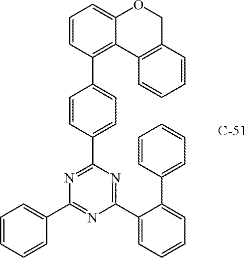 [SMILES: c1ccc(-c2nc(-c3ccc(-c4cccc5c4-c4ccccc4CO5)cc3)nc(-c3ccccc3-c3ccccc3)n2)cc1] 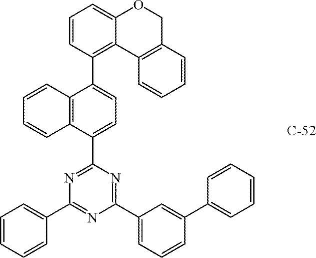 [SMILES: c1ccc(-c2cccc(-c3nc(-c4ccccc4)nc(-c4ccc(-c5cccc6c5-c5ccccc5CO6)c5ccccc45)n3)c2)cc1] 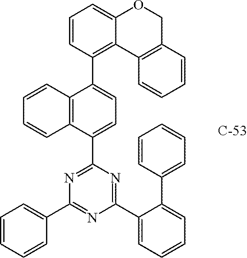 [SMILES: c1ccc(-c2nc(-c3ccccc3-c3ccccc3)nc(-c3ccc(-c4cccc5c4-c4ccccc4CO5)c4ccccc34)n2)cc1] 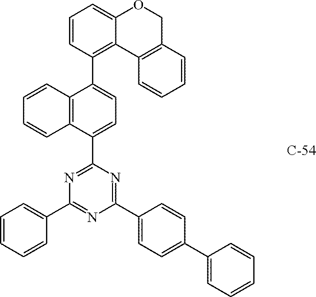 [SMILES: c1ccc(-c2ccc(-c3nc(-c4ccccc4)nc(-c4ccc(-c5cccc6c5-c5ccccc5CO6)c5ccccc45)n3)cc2)cc1] 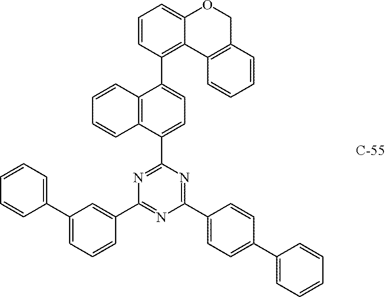 [SMILES: c1ccc(-c2ccc(-c3nc(-c4cccc(-c5ccccc5)c4)nc(-c4ccc(-c5cccc6c5-c5ccccc5CO6)c5ccccc45)n3)cc2)cc1] 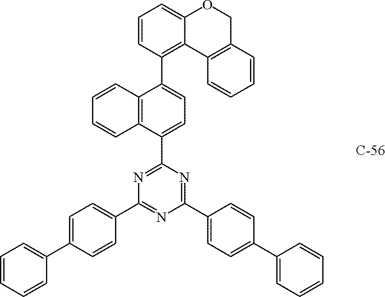 [SMILES: c1ccc(-c2ccc(-c3nc(-c4ccc(-c5ccccc5)cc4)nc(-c4ccc(-c5cccc6c5-c5ccccc5CO6)c5ccccc45)n3)cc2)cc1] 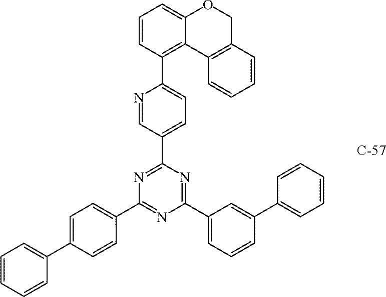 [SMILES: c1ccc(-c2ccc(-c3nc(-c4ccc(-c5cccc6c5-c5ccccc5CO6)nc4)nc(-c4cccc(-c5ccccc5)c4)n3)cc2)cc1] 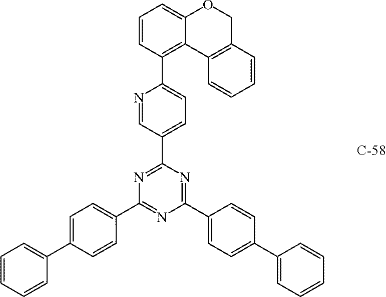 [SMILES: c1ccc(-c2ccc(-c3nc(-c4ccc(-c5ccccc5)cc4)nc(-c4ccc(-c5cccc6c5-c5ccccc5CO6)nc4)n3)cc2)cc1] 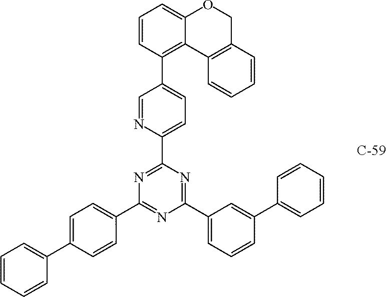 [SMILES: c1ccc(-c2ccc(-c3nc(-c4cccc(-c5ccccc5)c4)nc(-c4ccc(-c5cccc6c5-c5ccccc5CO6)cn4)n3)cc2)cc1] 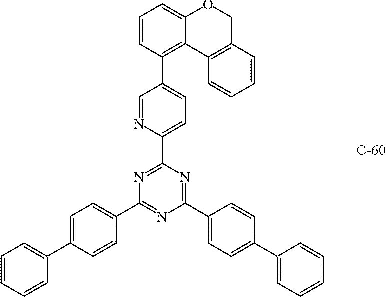 [SMILES: c1ccc(-c2ccc(-c3nc(-c4ccc(-c5ccccc5)cc4)nc(-c4ccc(-c5cccc6c5-c5ccccc5CO6)cn4)n3)cc2)cc1] 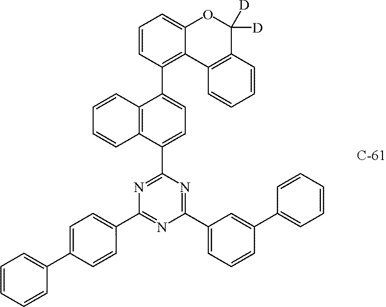 [SMILES: [2H]C1([2H])Oc2cccc(-c3ccc(-c4nc(-c5ccc(-c6ccccc6)cc5)nc(-c5cccc(-c6ccccc6)c5)n4)c4ccccc34)c2-c2ccccc21] 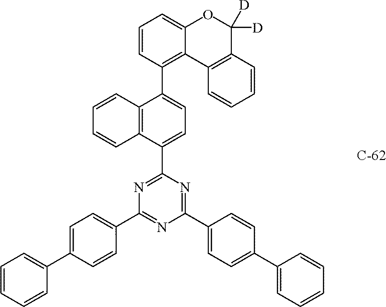 [SMILES: [2H]C1([2H])Oc2cccc(-c3ccc(-c4nc(-c5ccc(-c6ccccc6)cc5)nc(-c5ccc(-c6ccccc6)cc5)n4)c4ccccc34)c2-c2ccccc21] 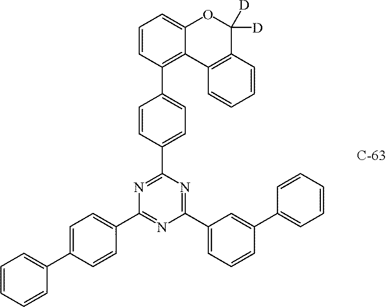 [SMILES: [2H]C1([2H])Oc2cccc(-c3ccc(-c4nc(-c5ccc(-c6ccccc6)cc5)nc(-c5cccc(-c6ccccc6)c5)n4)cc3)c2-c2ccccc21] 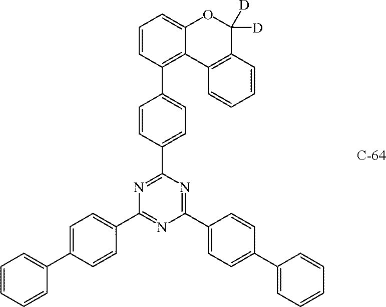 [SMILES: [2H]C1([2H])Oc2cccc(-c3ccc(-c4nc(-c5ccc(-c6ccccc6)cc5)nc(-c5ccc(-c6ccccc6)cc5)n4)cc3)c2-c2ccccc21] 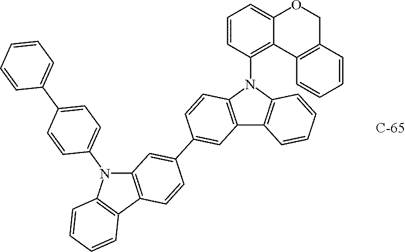 [SMILES: c1ccc(-c2ccc(-n3c4ccccc4c4ccc(-c5ccc6c(c5)c5ccccc5n6-c5cccc6c5-c5ccccc5CO6)cc43)cc2)cc1] 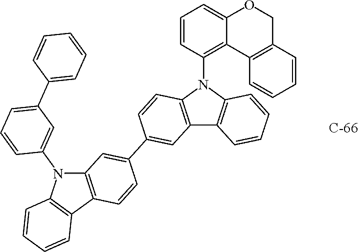 [SMILES: c1ccc(-c2cccc(-n3c4ccccc4c4ccc(-c5ccc6c(c5)c5ccccc5n6-c5cccc6c5-c5ccccc5CO6)cc43)c2)cc1] 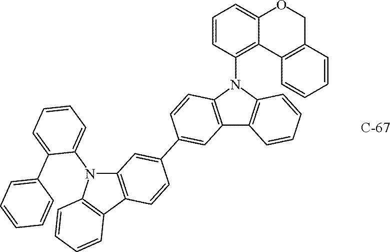 [SMILES: c1ccc(-c2ccccc2-n2c3ccccc3c3ccc(-c4ccc5c(c4)c4ccccc4n5-c4cccc5c4-c4ccccc4CO5)cc32)cc1] 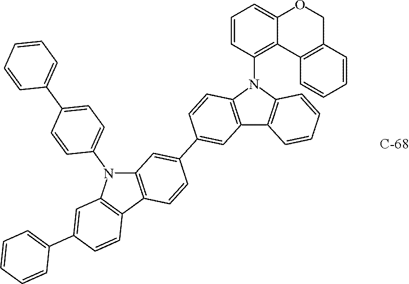 [SMILES: c1ccc(-c2ccc(-n3c4cc(-c5ccccc5)ccc4c4ccc(-c5ccc6c(c5)c5ccccc5n6-c5cccc6c5-c5ccccc5CO6)cc43)cc2)cc1] 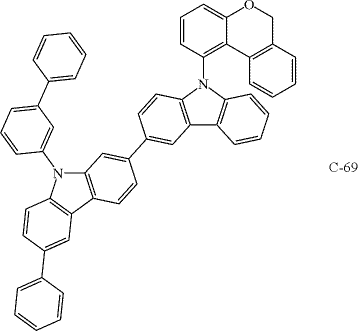 [SMILES: c1ccc(-c2cccc(-n3c4ccc(-c5ccccc5)cc4c4ccc(-c5ccc6c(c5)c5ccccc5n6-c5cccc6c5-c5ccccc5CO6)cc43)c2)cc1] 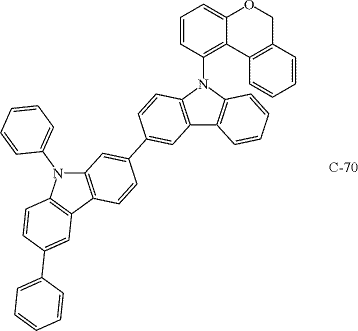 [SMILES: c1ccc(-c2ccc3c(c2)c2ccc(-c4ccc5c(c4)c4ccccc4n5-c4cccc5c4-c4ccccc4CO5)cc2n3-c2ccccc2)cc1] 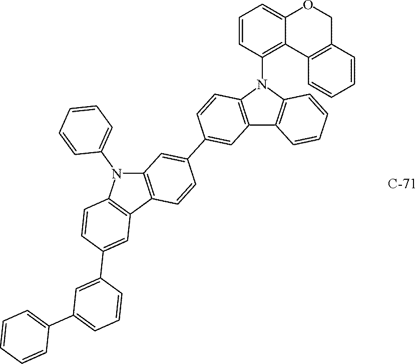 [SMILES: c1ccc(-c2cccc(-c3ccc4c(c3)c3ccc(-c5ccc6c(c5)c5ccccc5n6-c5cccc6c5-c5ccccc5CO6)cc3n4-c3ccccc3)c2)cc1] 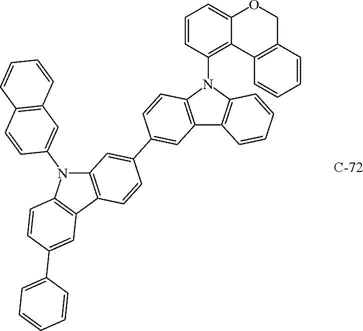 [SMILES: c1ccc(-c2ccc3c(c2)c2ccc(-c4ccc5c(c4)c4ccccc4n5-c4cccc5c4-c4ccccc4CO5)cc2n3-c2ccc3ccccc3c2)cc1] 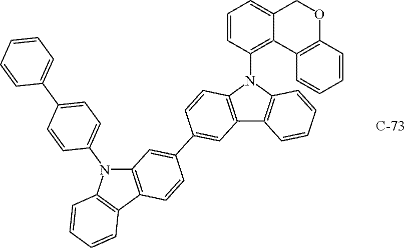 [SMILES: c1ccc(-c2ccc(-n3c4ccccc4c4ccc(-c5ccc6c(c5)c5ccccc5n6-c5cccc6c5-c5ccccc5OC6)cc43)cc2)cc1] 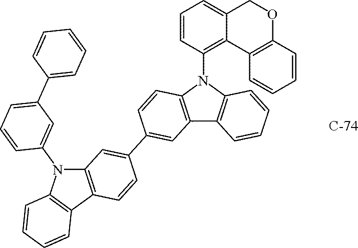 [SMILES: c1ccc(-c2cccc(-n3c4ccccc4c4ccc(-c5ccc6c(c5)c5ccccc5n6-c5cccc6c5-c5ccccc5OC6)cc43)c2)cc1] 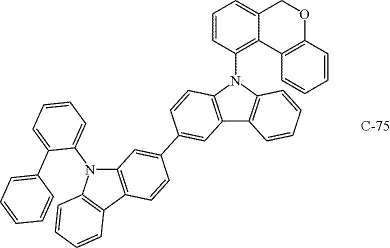 [SMILES: c1ccc(-c2ccccc2-n2c3ccccc3c3ccc(-c4ccc5c(c4)c4ccccc4n5-c4cccc5c4-c4ccccc4OC5)cc32)cc1] 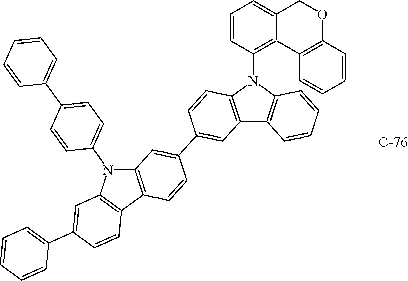 [SMILES: c1ccc(-c2ccc(-n3c4cc(-c5ccccc5)ccc4c4ccc(-c5ccc6c(c5)c5ccccc5n6-c5cccc6c5-c5ccccc5OC6)cc43)cc2)cc1] 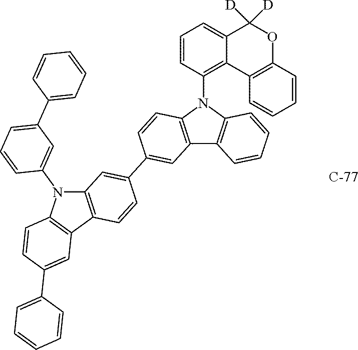 [SMILES: [2H]C1([2H])Oc2ccccc2-c2c(-n3c4ccccc4c4cc(-c5ccc6c7cc(-c8ccccc8)ccc7n(-c7cccc(-c8ccccc8)c7)c6c5)ccc43)cccc21] 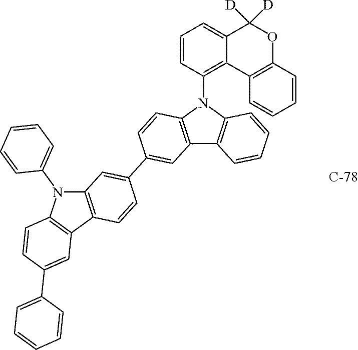 [SMILES: [2H]C1([2H])Oc2ccccc2-c2c(-n3c4ccccc4c4cc(-c5ccc6c7cc(-c8ccccc8)ccc7n(-c7ccccc7)c6c5)ccc43)cccc21] 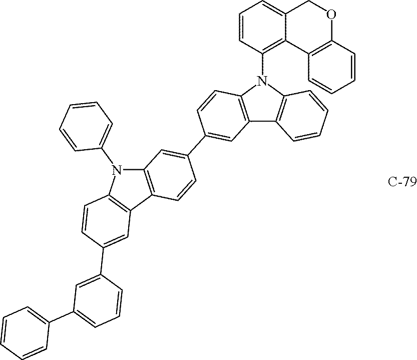 [SMILES: c1ccc(-c2cccc(-c3ccc4c(c3)c3ccc(-c5ccc6c(c5)c5ccccc5n6-c5cccc6c5-c5ccccc5OC6)cc3n4-c3ccccc3)c2)cc1] 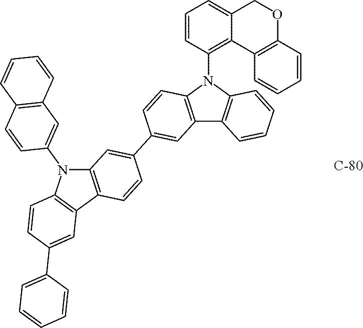 [SMILES: c1ccc(-c2ccc3c(c2)c2ccc(-c4ccc5c(c4)c4ccccc4n5-c4cccc5c4-c4ccccc4OC5)cc2n3-c2ccc3ccccc3c2)cc1] 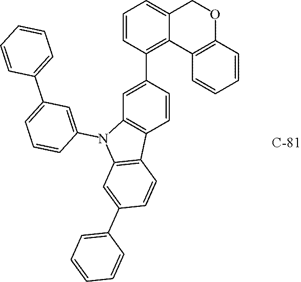 [SMILES: c1ccc(-c2cccc(-n3c4cc(-c5ccccc5)ccc4c4ccc(-c5cccc6c5-c5ccccc5OC6)cc43)c2)cc1] 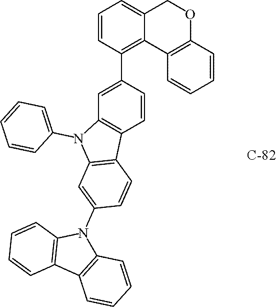 [SMILES: c1ccc(-n2c3cc(-c4cccc5c4-c4ccccc4OC5)ccc3c3ccc(-n4c5ccccc5c5ccccc54)cc32)cc1] 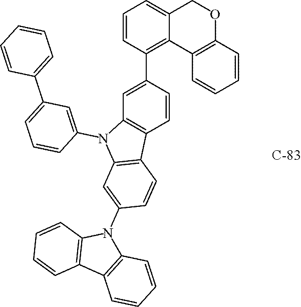 [SMILES: c1ccc(-c2cccc(-n3c4cc(-c5cccc6c5-c5ccccc5OC6)ccc4c4ccc(-n5c6ccccc6c6ccccc65)cc43)c2)cc1] 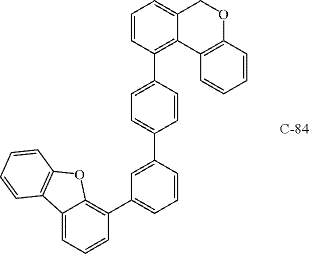 [SMILES: c1cc(-c2ccc(-c3cccc4c3-c3ccccc3OC4)cc2)cc(-c2cccc3c2oc2ccccc23)c1] 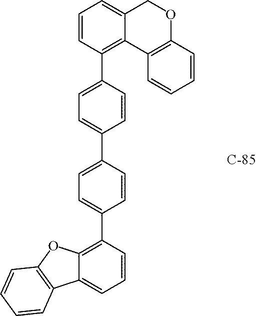 [SMILES: c1ccc2c(c1)OCc1cccc(-c3ccc(-c4ccc(-c5cccc6c5oc5ccccc56)cc4)cc3)c1-2] 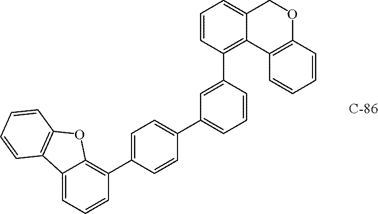 [SMILES: c1cc(-c2ccc(-c3cccc4c3oc3ccccc34)cc2)cc(-c2cccc3c2-c2ccccc2OC3)c1] 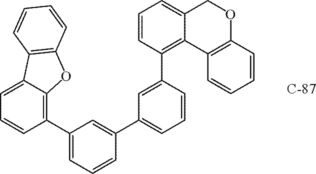 [SMILES: c1cc(-c2cccc(-c3cccc4c3oc3ccccc34)c2)cc(-c2cccc3c2-c2ccccc2OC3)c1] 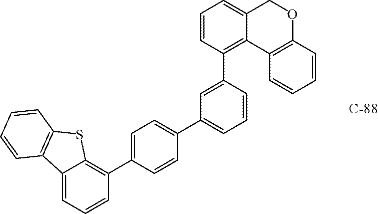 [SMILES: c1cc(-c2ccc(-c3cccc4c3sc3ccccc34)cc2)cc(-c2cccc3c2-c2ccccc2OC3)c1] 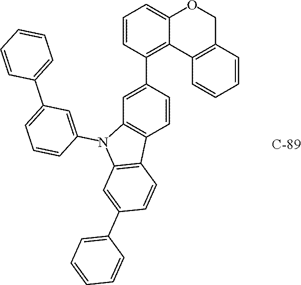 [SMILES: c1ccc(-c2cccc(-n3c4cc(-c5ccccc5)ccc4c4ccc(-c5cccc6c5-c5ccccc5CO6)cc43)c2)cc1] 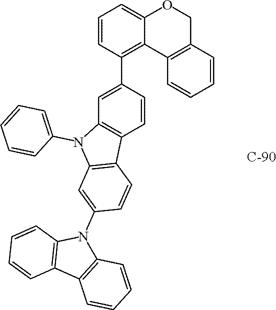 [SMILES: c1ccc(-n2c3cc(-c4cccc5c4-c4ccccc4CO5)ccc3c3ccc(-n4c5ccccc5c5ccccc54)cc32)cc1] 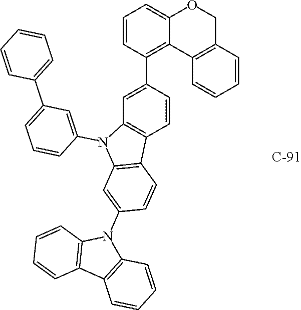 [SMILES: c1ccc(-c2cccc(-n3c4cc(-c5cccc6c5-c5ccccc5CO6)ccc4c4ccc(-n5c6ccccc6c6ccccc65)cc43)c2)cc1] 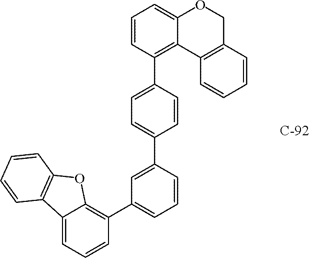 [SMILES: c1cc(-c2ccc(-c3cccc4c3-c3ccccc3CO4)cc2)cc(-c2cccc3c2oc2ccccc23)c1] 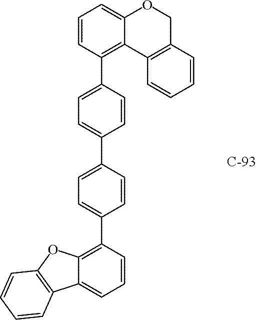 [SMILES: c1ccc2c(c1)COc1cccc(-c3ccc(-c4ccc(-c5cccc6c5oc5ccccc56)cc4)cc3)c1-2] 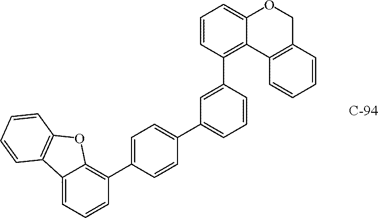 [SMILES: c1cc(-c2ccc(-c3cccc4c3oc3ccccc34)cc2)cc(-c2cccc3c2-c2ccccc2CO3)c1] 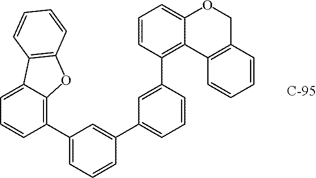 [SMILES: c1cc(-c2cccc(-c3cccc4c3oc3ccccc34)c2)cc(-c2cccc3c2-c2ccccc2CO3)c1] 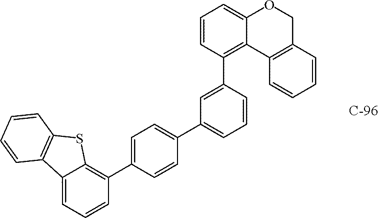 [SMILES: c1cc(-c2ccc(-c3cccc4c3sc3ccccc34)cc2)cc(-c2cccc3c2-c2ccccc2CO3)c1] 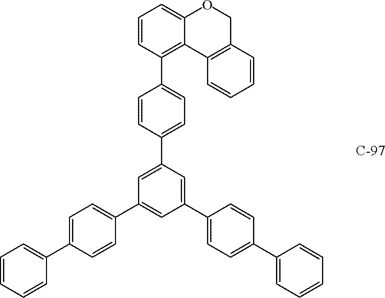 [SMILES: c1ccc(-c2ccc(-c3cc(-c4ccc(-c5ccccc5)cc4)cc(-c4ccc(-c5cccc6c5-c5ccccc5CO6)cc4)c3)cc2)cc1] 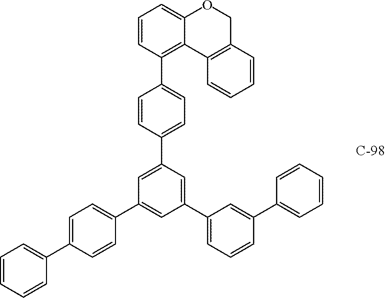 [SMILES: c1ccc(-c2ccc(-c3cc(-c4ccc(-c5cccc6c5-c5ccccc5CO6)cc4)cc(-c4cccc(-c5ccccc5)c4)c3)cc2)cc1] 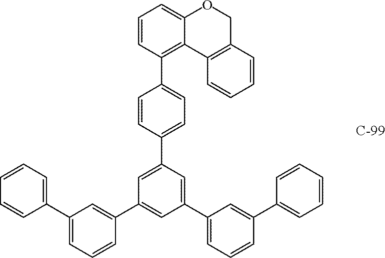 [SMILES: c1ccc(-c2cccc(-c3cc(-c4ccc(-c5cccc6c5-c5ccccc5CO6)cc4)cc(-c4cccc(-c5ccccc5)c4)c3)c2)cc1] 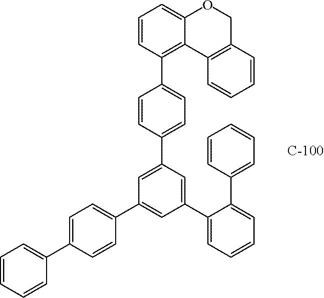 [SMILES: c1ccc(-c2ccc(-c3cc(-c4ccc(-c5cccc6c5-c5ccccc5CO6)cc4)cc(-c4ccccc4-c4ccccc4)c3)cc2)cc1] 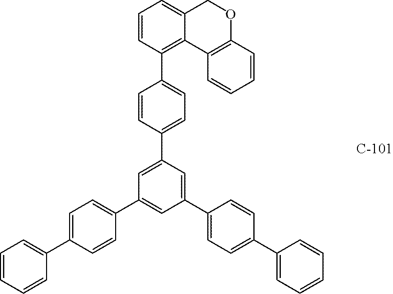 [SMILES: c1ccc(-c2ccc(-c3cc(-c4ccc(-c5ccccc5)cc4)cc(-c4ccc(-c5cccc6c5-c5ccccc5OC6)cc4)c3)cc2)cc1] 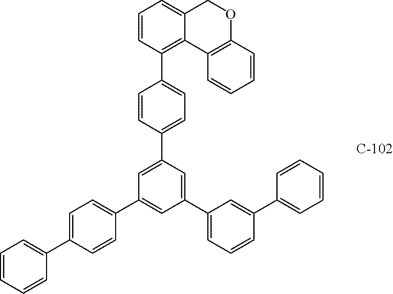 [SMILES: c1ccc(-c2ccc(-c3cc(-c4ccc(-c5cccc6c5-c5ccccc5OC6)cc4)cc(-c4cccc(-c5ccccc5)c4)c3)cc2)cc1] 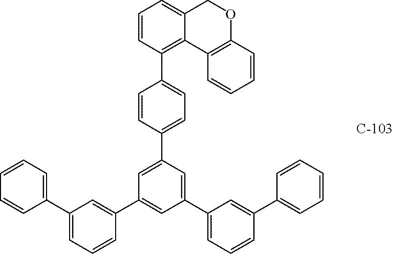 [SMILES: c1ccc(-c2cccc(-c3cc(-c4ccc(-c5cccc6c5-c5ccccc5OC6)cc4)cc(-c4cccc(-c5ccccc5)c4)c3)c2)cc1] 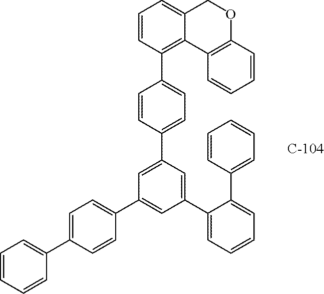 [SMILES: c1ccc(-c2ccc(-c3cc(-c4ccc(-c5cccc6c5-c5ccccc5OC6)cc4)cc(-c4ccccc4-c4ccccc4)c3)cc2)cc1] 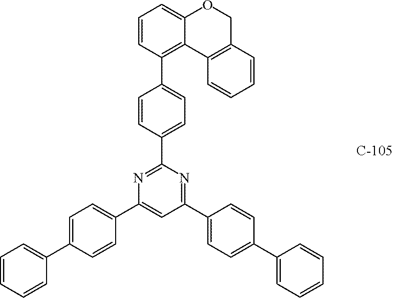 [SMILES: c1ccc(-c2ccc(-c3cc(-c4ccc(-c5ccccc5)cc4)nc(-c4ccc(-c5cccc6c5-c5ccccc5CO6)cc4)n3)cc2)cc1] 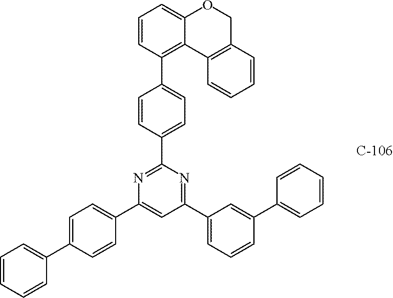 [SMILES: c1ccc(-c2ccc(-c3cc(-c4cccc(-c5ccccc5)c4)nc(-c4ccc(-c5cccc6c5-c5ccccc5CO6)cc4)n3)cc2)cc1] 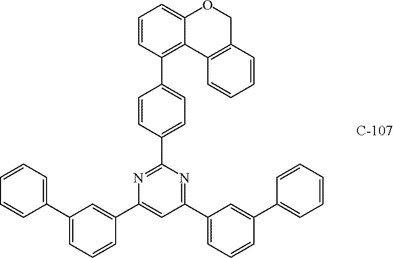 [SMILES: c1ccc(-c2cccc(-c3cc(-c4cccc(-c5ccccc5)c4)nc(-c4ccc(-c5cccc6c5-c5ccccc5CO6)cc4)n3)c2)cc1] 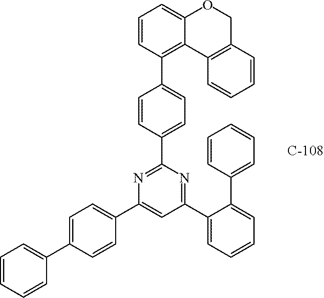 [SMILES: c1ccc(-c2ccc(-c3cc(-c4ccccc4-c4ccccc4)nc(-c4ccc(-c5cccc6c5-c5ccccc5CO6)cc4)n3)cc2)cc1] 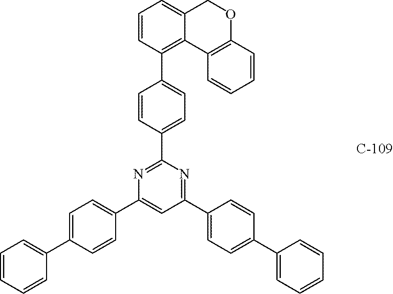 [SMILES: c1ccc(-c2ccc(-c3cc(-c4ccc(-c5ccccc5)cc4)nc(-c4ccc(-c5cccc6c5-c5ccccc5OC6)cc4)n3)cc2)cc1] 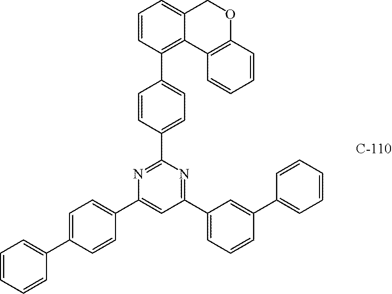 [SMILES: c1ccc(-c2ccc(-c3cc(-c4cccc(-c5ccccc5)c4)nc(-c4ccc(-c5cccc6c5-c5ccccc5OC6)cc4)n3)cc2)cc1] 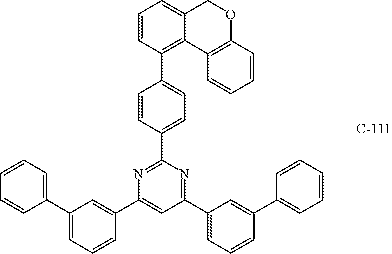 [SMILES: c1ccc(-c2cccc(-c3cc(-c4cccc(-c5ccccc5)c4)nc(-c4ccc(-c5cccc6c5-c5ccccc5OC6)cc4)n3)c2)cc1] 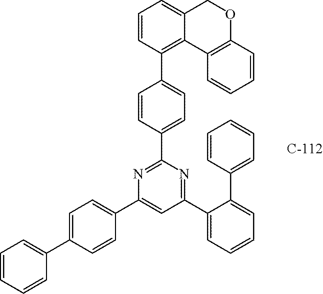 [SMILES: c1ccc(-c2ccc(-c3cc(-c4ccccc4-c4ccccc4)nc(-c4ccc(-c5cccc6c5-c5ccccc5OC6)cc4)n3)cc2)cc1] 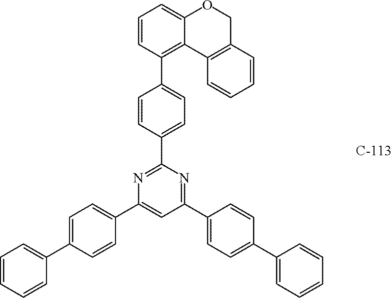 [SMILES: c1ccc(-c2ccc(-c3cc(-c4ccc(-c5ccccc5)cc4)nc(-c4ccc(-c5cccc6c5-c5ccccc5CO6)cc4)n3)cc2)cc1] 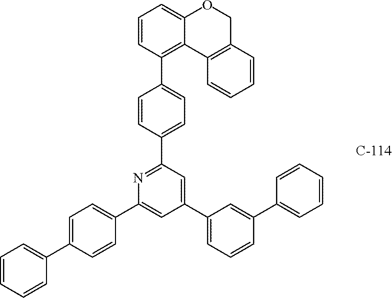 [SMILES: c1ccc(-c2ccc(-c3cc(-c4cccc(-c5ccccc5)c4)cc(-c4ccc(-c5cccc6c5-c5ccccc5CO6)cc4)n3)cc2)cc1] 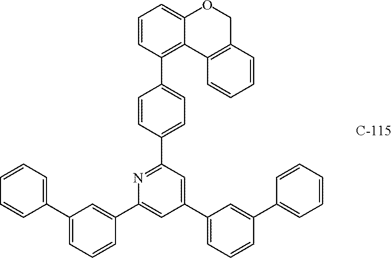 [SMILES: c1ccc(-c2cccc(-c3cc(-c4ccc(-c5cccc6c5-c5ccccc5CO6)cc4)nc(-c4cccc(-c5ccccc5)c4)c3)c2)cc1] 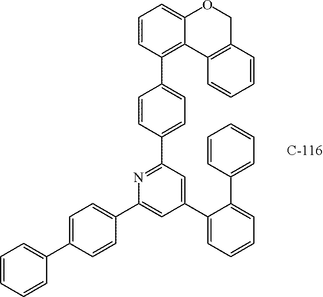 [SMILES: c1ccc(-c2ccc(-c3cc(-c4ccccc4-c4ccccc4)cc(-c4ccc(-c5cccc6c5-c5ccccc5CO6)cc4)n3)cc2)cc1] 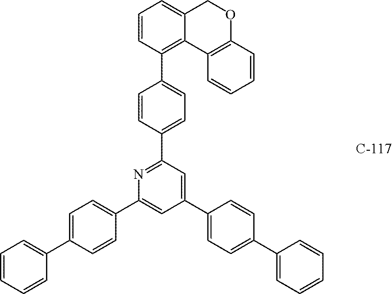 [SMILES: c1ccc(-c2ccc(-c3cc(-c4ccc(-c5ccccc5)cc4)nc(-c4ccc(-c5cccc6c5-c5ccccc5OC6)cc4)c3)cc2)cc1] 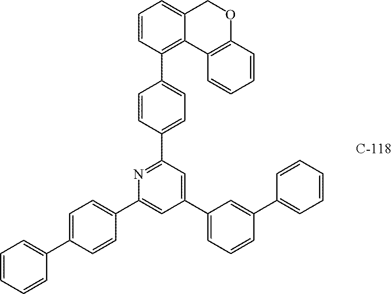 [SMILES: c1ccc(-c2ccc(-c3cc(-c4cccc(-c5ccccc5)c4)cc(-c4ccc(-c5cccc6c5-c5ccccc5OC6)cc4)n3)cc2)cc1] 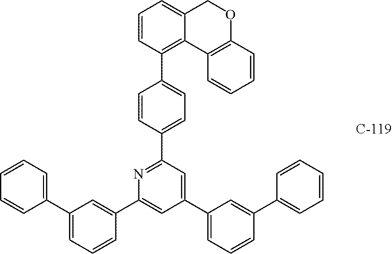 [SMILES: c1ccc(-c2cccc(-c3cc(-c4ccc(-c5cccc6c5-c5ccccc5OC6)cc4)nc(-c4cccc(-c5ccccc5)c4)c3)c2)cc1] 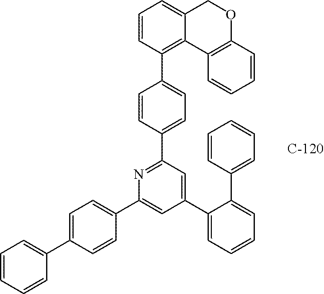 [SMILES: c1ccc(-c2ccc(-c3cc(-c4ccccc4-c4ccccc4)cc(-c4ccc(-c5cccc6c5-c5ccccc5OC6)cc4)n3)cc2)cc1] 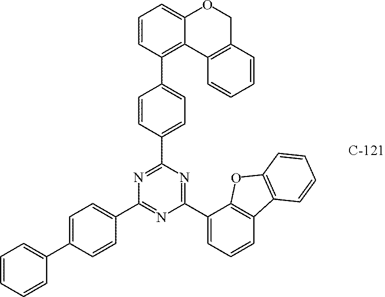 [SMILES: c1ccc(-c2ccc(-c3nc(-c4ccc(-c5cccc6c5-c5ccccc5CO6)cc4)nc(-c4cccc5c4oc4ccccc45)n3)cc2)cc1] 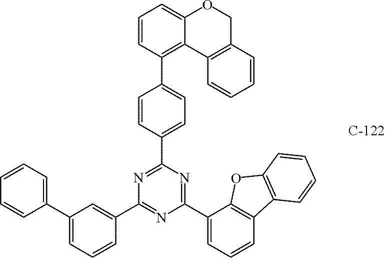 [SMILES: c1ccc(-c2cccc(-c3nc(-c4ccc(-c5cccc6c5-c5ccccc5CO6)cc4)nc(-c4cccc5c4oc4ccccc45)n3)c2)cc1] 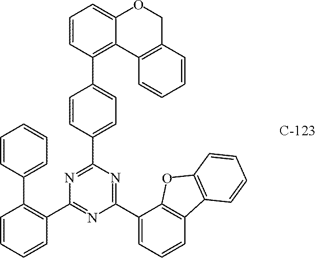 [SMILES: c1ccc(-c2ccccc2-c2nc(-c3ccc(-c4cccc5c4-c4ccccc4CO5)cc3)nc(-c3cccc4c3oc3ccccc34)n2)cc1] 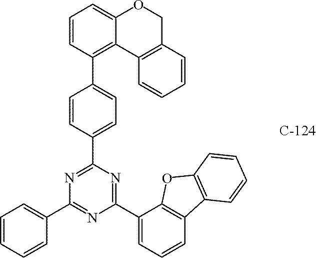 [SMILES: c1ccc(-c2nc(-c3ccc(-c4cccc5c4-c4ccccc4CO5)cc3)nc(-c3cccc4c3oc3ccccc34)n2)cc1] 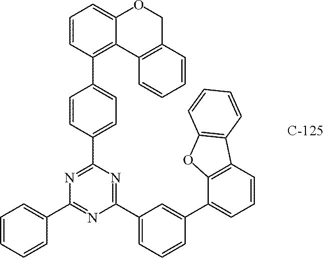 [SMILES: c1ccc(-c2nc(-c3ccc(-c4cccc5c4-c4ccccc4CO5)cc3)nc(-c3cccc(-c4cccc5c4oc4ccccc45)c3)n2)cc1] 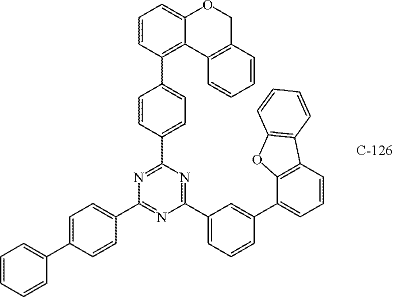 [SMILES: c1ccc(-c2ccc(-c3nc(-c4ccc(-c5cccc6c5-c5ccccc5CO6)cc4)nc(-c4cccc(-c5cccc6c5oc5ccccc56)c4)n3)cc2)cc1] 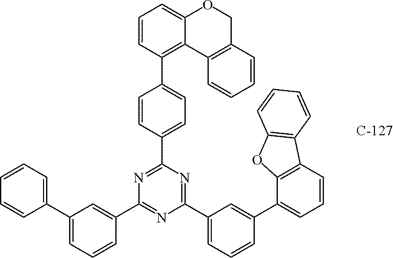 [SMILES: c1ccc(-c2cccc(-c3nc(-c4ccc(-c5cccc6c5-c5ccccc5CO6)cc4)nc(-c4cccc(-c5cccc6c5oc5ccccc56)c4)n3)c2)cc1] 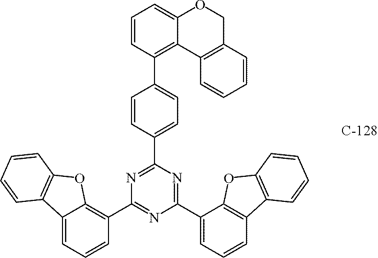 [SMILES: c1ccc2c(c1)COc1cccc(-c3ccc(-c4nc(-c5cccc6c5oc5ccccc56)nc(-c5cccc6c5oc5ccccc56)n4)cc3)c1-2] 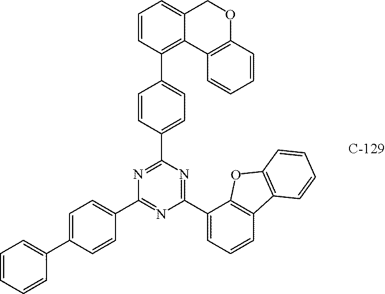 [SMILES: c1ccc(-c2ccc(-c3nc(-c4ccc(-c5cccc6c5-c5ccccc5OC6)cc4)nc(-c4cccc5c4oc4ccccc45)n3)cc2)cc1] 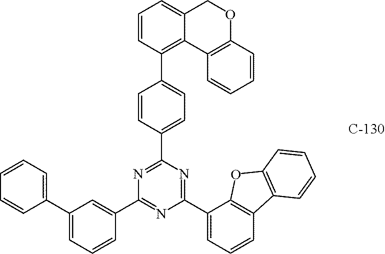 [SMILES: c1ccc(-c2cccc(-c3nc(-c4ccc(-c5cccc6c5-c5ccccc5OC6)cc4)nc(-c4cccc5c4oc4ccccc45)n3)c2)cc1] 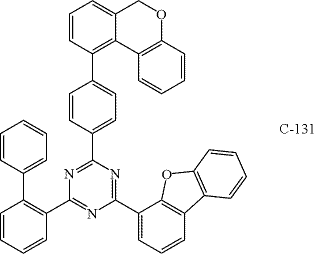 [SMILES: c1ccc(-c2ccccc2-c2nc(-c3ccc(-c4cccc5c4-c4ccccc4OC5)cc3)nc(-c3cccc4c3oc3ccccc34)n2)cc1] 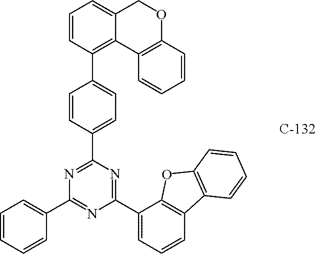 [SMILES: c1ccc(-c2nc(-c3ccc(-c4cccc5c4-c4ccccc4OC5)cc3)nc(-c3cccc4c3oc3ccccc34)n2)cc1] 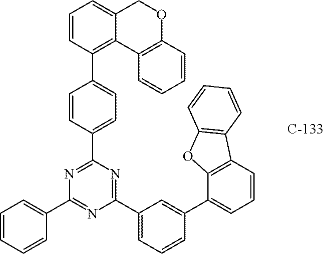 [SMILES: c1ccc(-c2nc(-c3ccc(-c4cccc5c4-c4ccccc4OC5)cc3)nc(-c3cccc(-c4cccc5c4oc4ccccc45)c3)n2)cc1] 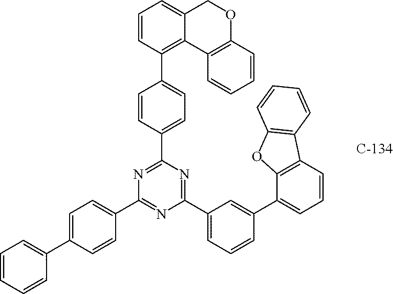 [SMILES: c1ccc(-c2ccc(-c3nc(-c4ccc(-c5cccc6c5-c5ccccc5OC6)cc4)nc(-c4cccc(-c5cccc6c5oc5ccccc56)c4)n3)cc2)cc1] 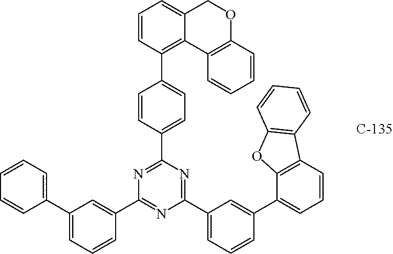 [SMILES: c1ccc(-c2cccc(-c3nc(-c4ccc(-c5cccc6c5-c5ccccc5OC6)cc4)nc(-c4cccc(-c5cccc6c5oc5ccccc56)c4)n3)c2)cc1] 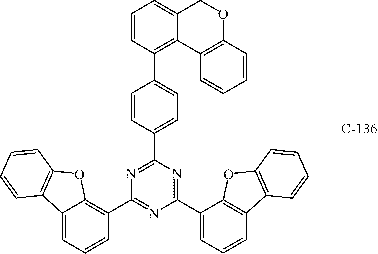 [SMILES: c1ccc2c(c1)OCc1cccc(-c3ccc(-c4nc(-c5cccc6c5oc5ccccc56)nc(-c5cccc6c5oc5ccccc56)n4)cc3)c1-2] 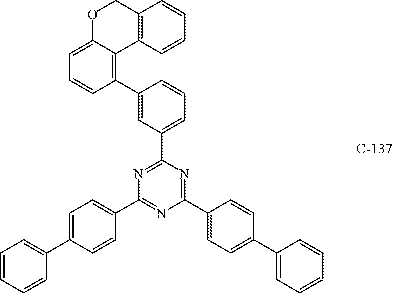 [SMILES: c1ccc(-c2ccc(-c3nc(-c4ccc(-c5ccccc5)cc4)nc(-c4cccc(-c5cccc6c5-c5ccccc5CO6)c4)n3)cc2)cc1] 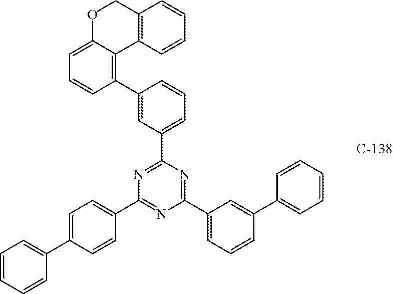 [SMILES: c1ccc(-c2ccc(-c3nc(-c4cccc(-c5ccccc5)c4)nc(-c4cccc(-c5cccc6c5-c5ccccc5CO6)c4)n3)cc2)cc1] 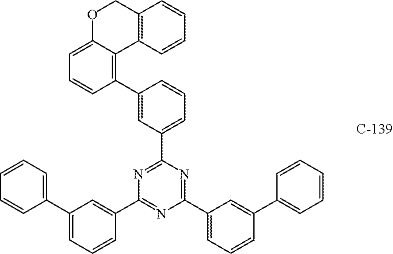 [SMILES: c1ccc(-c2cccc(-c3nc(-c4cccc(-c5ccccc5)c4)nc(-c4cccc(-c5cccc6c5-c5ccccc5CO6)c4)n3)c2)cc1] 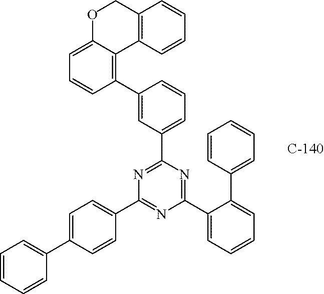 [SMILES: c1ccc(-c2ccc(-c3nc(-c4cccc(-c5cccc6c5-c5ccccc5CO6)c4)nc(-c4ccccc4-c4ccccc4)n3)cc2)cc1] 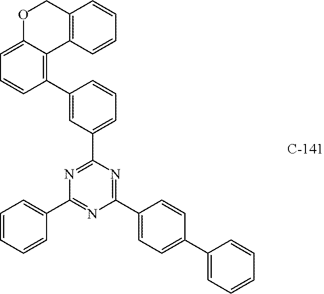 [SMILES: c1ccc(-c2ccc(-c3nc(-c4ccccc4)nc(-c4cccc(-c5cccc6c5-c5ccccc5CO6)c4)n3)cc2)cc1] 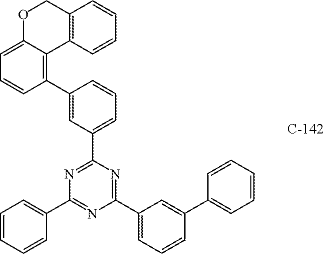 [SMILES: c1ccc(-c2cccc(-c3nc(-c4ccccc4)nc(-c4cccc(-c5cccc6c5-c5ccccc5CO6)c4)n3)c2)cc1] 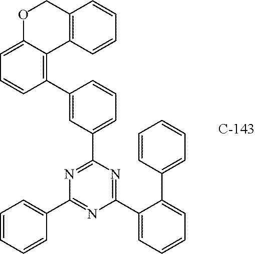 [SMILES: c1ccc(-c2nc(-c3cccc(-c4cccc5c4-c4ccccc4CO5)c3)nc(-c3ccccc3-c3ccccc3)n2)cc1] 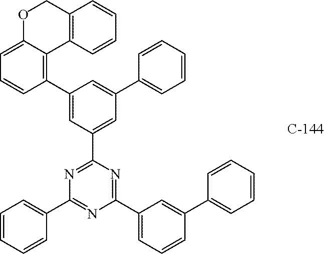 [SMILES: c1ccc(-c2cccc(-c3nc(-c4ccccc4)nc(-c4cc(-c5ccccc5)cc(-c5cccc6c5-c5ccccc5CO6)c4)n3)c2)cc1] 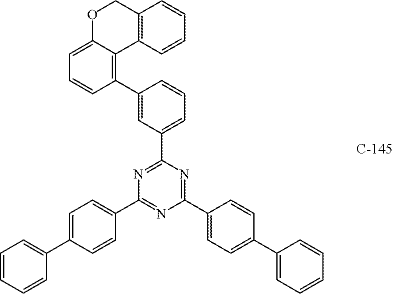 [SMILES: c1ccc(-c2ccc(-c3nc(-c4ccc(-c5ccccc5)cc4)nc(-c4cccc(-c5cccc6c5-c5ccccc5CO6)c4)n3)cc2)cc1] 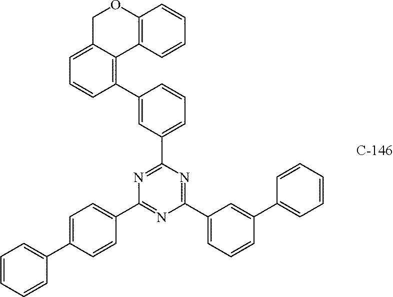 [SMILES: c1ccc(-c2ccc(-c3nc(-c4cccc(-c5ccccc5)c4)nc(-c4cccc(-c5cccc6c5-c5ccccc5OC6)c4)n3)cc2)cc1] 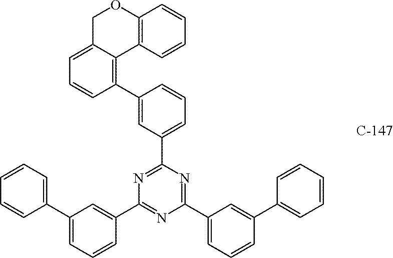 [SMILES: c1ccc(-c2cccc(-c3nc(-c4cccc(-c5ccccc5)c4)nc(-c4cccc(-c5cccc6c5-c5ccccc5OC6)c4)n3)c2)cc1] 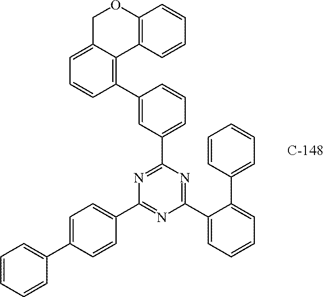 [SMILES: c1ccc(-c2ccc(-c3nc(-c4cccc(-c5cccc6c5-c5ccccc5OC6)c4)nc(-c4ccccc4-c4ccccc4)n3)cc2)cc1] 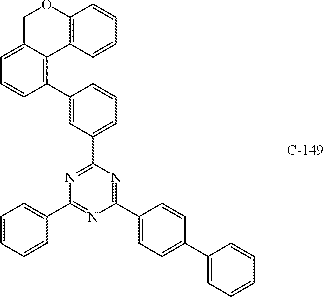 [SMILES: c1ccc(-c2ccc(-c3nc(-c4ccccc4)nc(-c4cccc(-c5cccc6c5-c5ccccc5OC6)c4)n3)cc2)cc1] 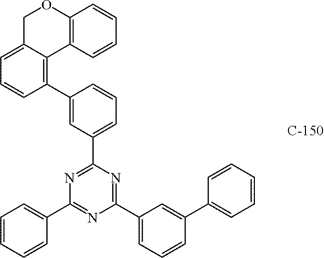 [SMILES: c1ccc(-c2cccc(-c3nc(-c4ccccc4)nc(-c4cccc(-c5cccc6c5-c5ccccc5OC6)c4)n3)c2)cc1] 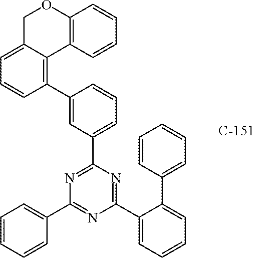 [SMILES: c1ccc(-c2nc(-c3cccc(-c4cccc5c4-c4ccccc4OC5)c3)nc(-c3ccccc3-c3ccccc3)n2)cc1] 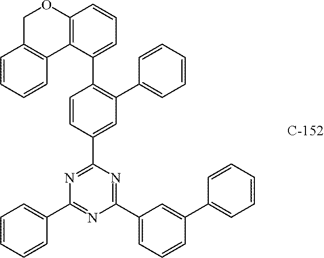 [SMILES: c1ccc(-c2cccc(-c3nc(-c4ccccc4)nc(-c4ccc(-c5cccc6c5-c5ccccc5CO6)c(-c5ccccc5)c4)n3)c2)cc1] 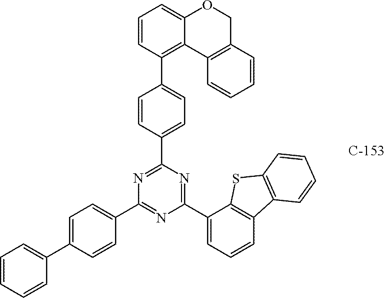 [SMILES: c1ccc(-c2ccc(-c3nc(-c4ccc(-c5cccc6c5-c5ccccc5CO6)cc4)nc(-c4cccc5c4sc4ccccc45)n3)cc2)cc1] 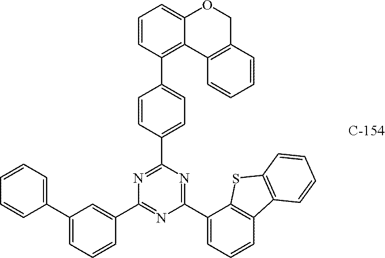 [SMILES: c1ccc(-c2cccc(-c3nc(-c4ccc(-c5cccc6c5-c5ccccc5CO6)cc4)nc(-c4cccc5c4sc4ccccc45)n3)c2)cc1] 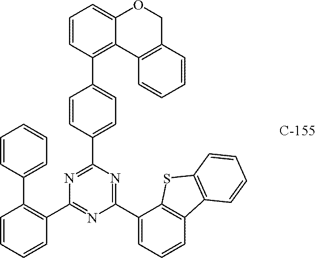 [SMILES: c1ccc(-c2ccccc2-c2nc(-c3ccc(-c4cccc5c4-c4ccccc4CO5)cc3)nc(-c3cccc4c3sc3ccccc34)n2)cc1] 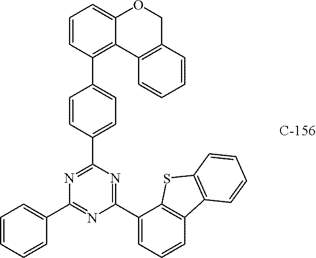 [SMILES: c1ccc(-c2nc(-c3ccc(-c4cccc5c4-c4ccccc4CO5)cc3)nc(-c3cccc4c3sc3ccccc34)n2)cc1] 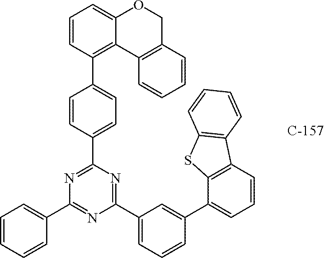 [SMILES: c1ccc(-c2nc(-c3ccc(-c4cccc5c4-c4ccccc4CO5)cc3)nc(-c3cccc(-c4cccc5c4sc4ccccc45)c3)n2)cc1] 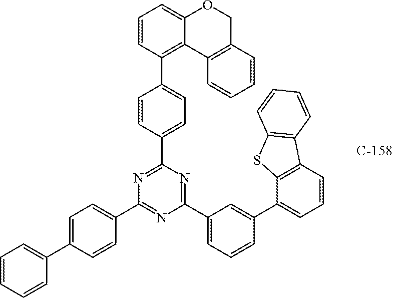 [SMILES: c1ccc(-c2ccc(-c3nc(-c4ccc(-c5cccc6c5-c5ccccc5CO6)cc4)nc(-c4cccc(-c5cccc6c5sc5ccccc56)c4)n3)cc2)cc1] 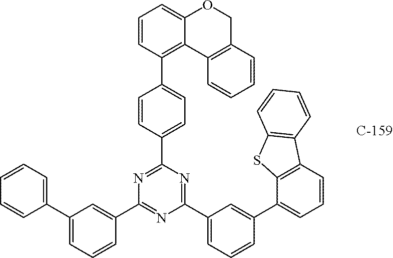 [SMILES: c1ccc(-c2cccc(-c3nc(-c4ccc(-c5cccc6c5-c5ccccc5CO6)cc4)nc(-c4cccc(-c5cccc6c5sc5ccccc56)c4)n3)c2)cc1] 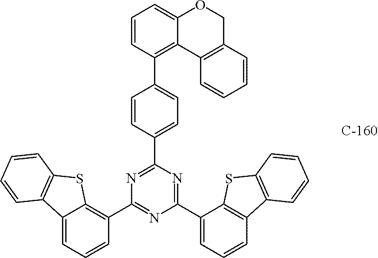 [SMILES: c1ccc2c(c1)COc1cccc(-c3ccc(-c4nc(-c5cccc6c5sc5ccccc56)nc(-c5cccc6c5sc5ccccc56)n4)cc3)c1-2] 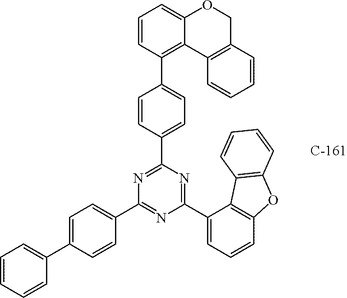 [SMILES: c1ccc(-c2ccc(-c3nc(-c4ccc(-c5cccc6c5-c5ccccc5CO6)cc4)nc(-c4cccc5oc6ccccc6c45)n3)cc2)cc1] 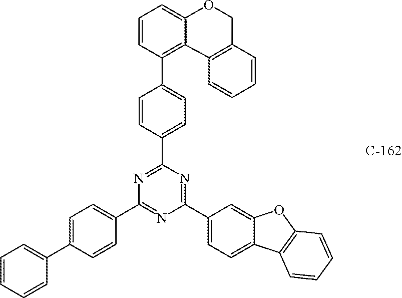 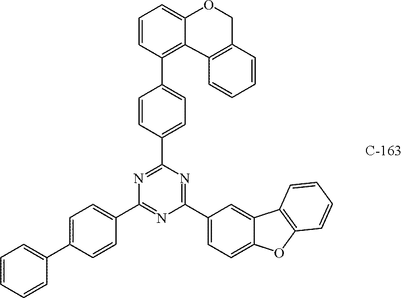 [SMILES: c1ccc(-c2ccc(-c3nc(-c4ccc(-c5cccc6c5-c5ccccc5CO6)cc4)nc(-c4ccc5oc6ccccc6c5c4)n3)cc2)cc1] 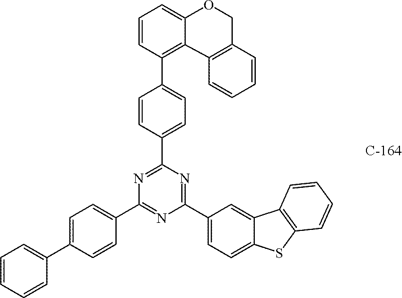 [SMILES: c1ccc(-c2ccc(-c3nc(-c4ccc(-c5cccc6c5-c5ccccc5CO6)cc4)nc(-c4ccc5sc6ccccc6c5c4)n3)cc2)cc1] 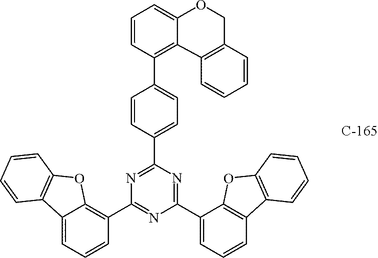 [SMILES: c1ccc2c(c1)COc1cccc(-c3ccc(-c4nc(-c5cccc6c5oc5ccccc56)nc(-c5cccc6c5oc5ccccc56)n4)cc3)c1-2] 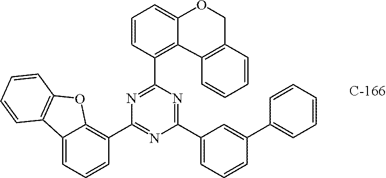 [SMILES: c1ccc(-c2cccc(-c3nc(-c4cccc5c4-c4ccccc4CO5)nc(-c4cccc5c4oc4ccccc45)n3)c2)cc1] 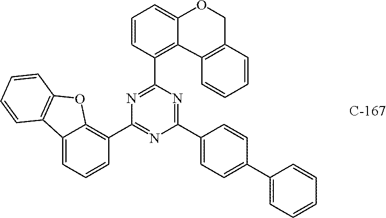 [SMILES: c1ccc(-c2ccc(-c3nc(-c4cccc5c4-c4ccccc4CO5)nc(-c4cccc5c4oc4ccccc45)n3)cc2)cc1] 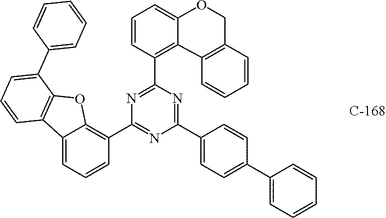 [SMILES: c1ccc(-c2ccc(-c3nc(-c4cccc5c4-c4ccccc4CO5)nc(-c4cccc5c4oc4c(-c6ccccc6)cccc45)n3)cc2)cc1] 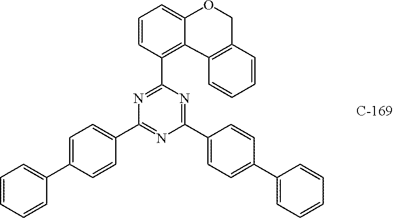 [SMILES: c1ccc(-c2ccc(-c3nc(-c4ccc(-c5ccccc5)cc4)nc(-c4cccc5c4-c4ccccc4CO5)n3)cc2)cc1] 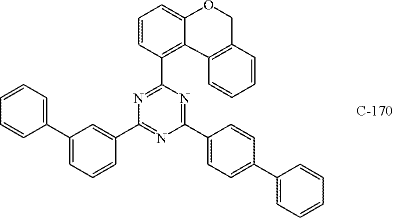 [SMILES: c1ccc(-c2ccc(-c3nc(-c4cccc(-c5ccccc5)c4)nc(-c4cccc5c4-c4ccccc4CO5)n3)cc2)cc1] 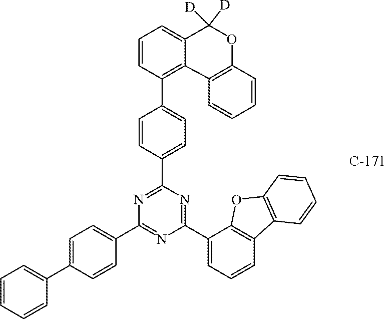 [SMILES: [2H]C1([2H])Oc2ccccc2-c2c(-c3ccc(-c4nc(-c5ccc(-c6ccccc6)cc5)nc(-c5cccc6c5oc5ccccc56)n4)cc3)cccc21] 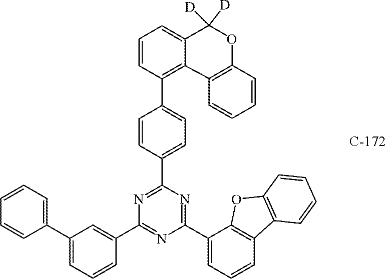 [SMILES: [2H]C1([2H])Oc2ccccc2-c2c(-c3ccc(-c4nc(-c5cccc(-c6ccccc6)c5)nc(-c5cccc6c5oc5ccccc56)n4)cc3)cccc21] 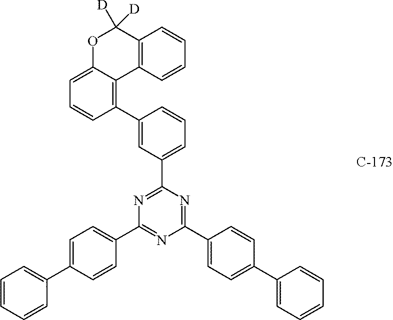 [SMILES: [2H]C1([2H])Oc2cccc(-c3cccc(-c4nc(-c5ccc(-c6ccccc6)cc5)nc(-c5ccc(-c6ccccc6)cc5)n4)c3)c2-c2ccccc21] 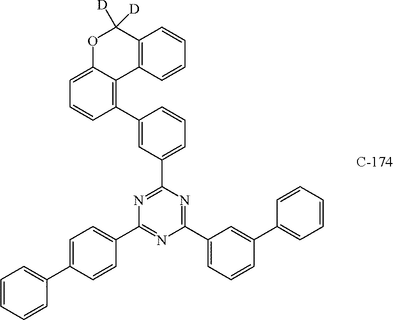 [SMILES: [2H]C1([2H])Oc2cccc(-c3cccc(-c4nc(-c5ccc(-c6ccccc6)cc5)nc(-c5cccc(-c6ccccc6)c5)n4)c3)c2-c2ccccc21] 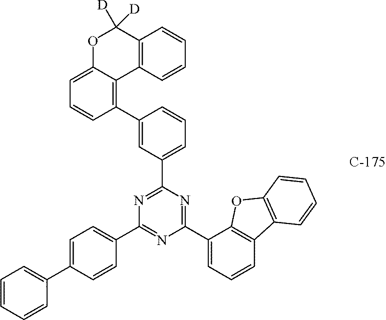 [SMILES: [2H]C1([2H])Oc2cccc(-c3cccc(-c4nc(-c5ccc(-c6ccccc6)cc5)nc(-c5cccc6c5oc5ccccc56)n4)c3)c2-c2ccccc21] 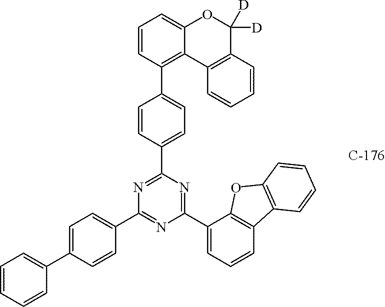 [SMILES: [2H]C1([2H])Oc2cccc(-c3ccc(-c4nc(-c5ccc(-c6ccccc6)cc5)nc(-c5cccc6c5oc5ccccc56)n4)cc3)c2-c2ccccc21] 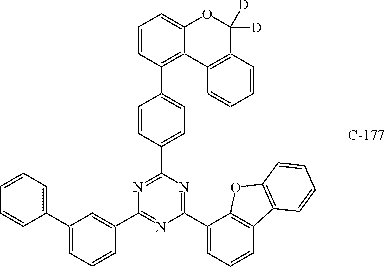 [SMILES: [2H]C1([2H])Oc2cccc(-c3ccc(-c4nc(-c5cccc(-c6ccccc6)c5)nc(-c5cccc6c5oc5ccccc56)n4)cc3)c2-c2ccccc21] 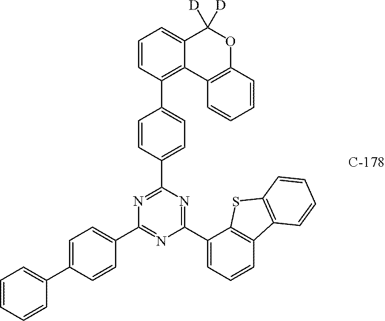 [SMILES: [2H]C1([2H])Oc2ccccc2-c2c(-c3ccc(-c4nc(-c5ccc(-c6ccccc6)cc5)nc(-c5cccc6c5sc5ccccc56)n4)cc3)cccc21] 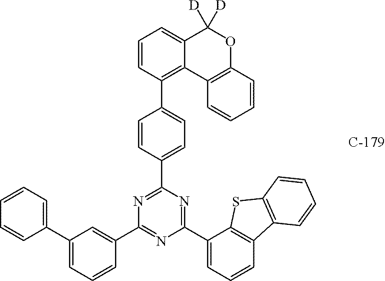 [SMILES: [2H]C1([2H])Oc2ccccc2-c2c(-c3ccc(-c4nc(-c5cccc(-c6ccccc6)c5)nc(-c5cccc6c5sc5ccccc56)n4)cc3)cccc21] 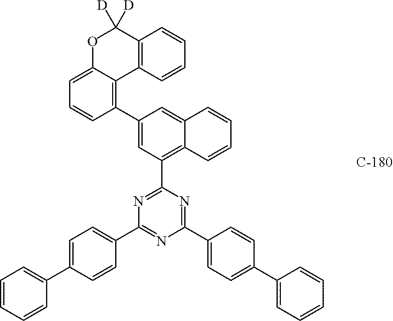 [SMILES: [2H]C1([2H])Oc2cccc(-c3cc(-c4nc(-c5ccc(-c6ccccc6)cc5)nc(-c5ccc(-c6ccccc6)cc5)n4)c4ccccc4c3)c2-c2ccccc21] 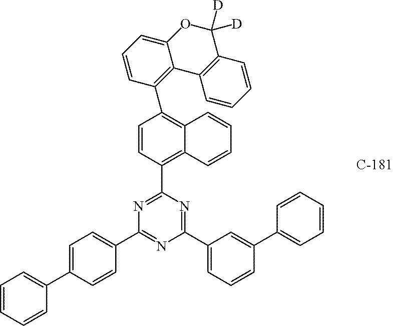 [SMILES: [2H]C1([2H])Oc2cccc(-c3ccc(-c4nc(-c5ccc(-c6ccccc6)cc5)nc(-c5cccc(-c6ccccc6)c5)n4)c4ccccc34)c2-c2ccccc21] 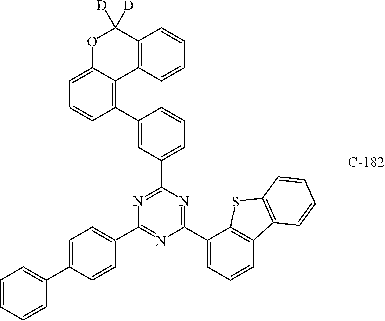 [SMILES: [2H]C1([2H])Oc2cccc(-c3cccc(-c4nc(-c5ccc(-c6ccccc6)cc5)nc(-c5cccc6c5sc5ccccc56)n4)c3)c2-c2ccccc21] 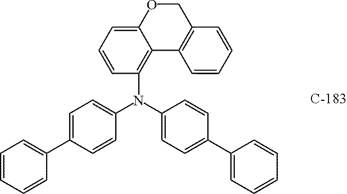 [SMILES: c1ccc(-c2ccc(N(c3ccc(-c4ccccc4)cc3)c3cccc4c3-c3ccccc3CO4)cc2)cc1] 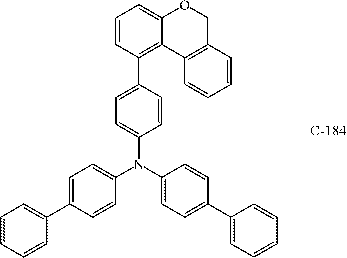 [SMILES: c1ccc(-c2ccc(N(c3ccc(-c4ccccc4)cc3)c3ccc(-c4cccc5c4-c4ccccc4CO5)cc3)cc2)cc1] 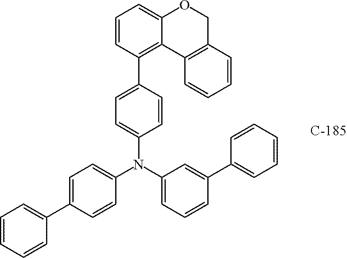 [SMILES: c1ccc(-c2ccc(N(c3ccc(-c4cccc5c4-c4ccccc4CO5)cc3)c3cccc(-c4ccccc4)c3)cc2)cc1] 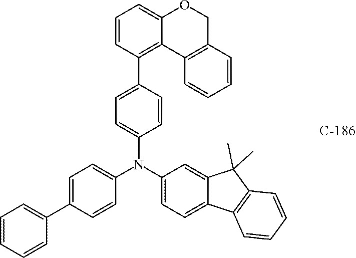 [SMILES: CC1(C)c2ccccc2-c2ccc(N(c3ccc(-c4ccccc4)cc3)c3ccc(-c4cccc5c4-c4ccccc4CO5)cc3)cc21] 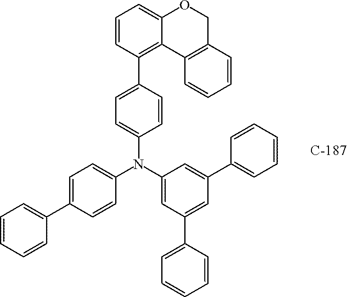 [SMILES: c1ccc(-c2ccc(N(c3ccc(-c4cccc5c4-c4ccccc4CO5)cc3)c3cc(-c4ccccc4)cc(-c4ccccc4)c3)cc2)cc1] 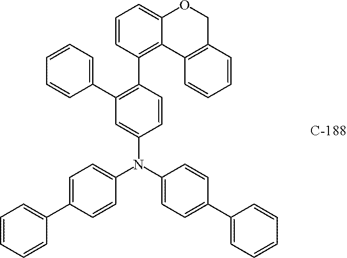 [SMILES: c1ccc(-c2ccc(N(c3ccc(-c4ccccc4)cc3)c3ccc(-c4cccc5c4-c4ccccc4CO5)c(-c4ccccc4)c3)cc2)cc1] 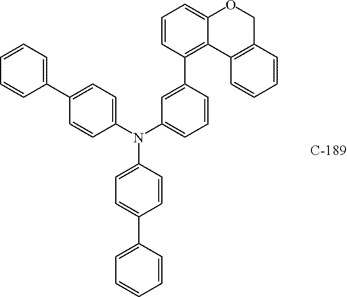 [SMILES: c1ccc(-c2ccc(N(c3ccc(-c4ccccc4)cc3)c3cccc(-c4cccc5c4-c4ccccc4CO5)c3)cc2)cc1] 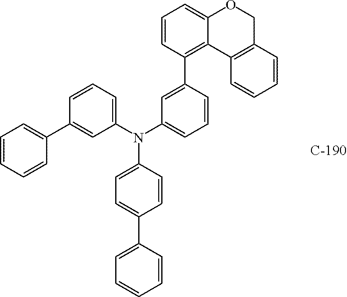 [SMILES: c1ccc(-c2ccc(N(c3cccc(-c4ccccc4)c3)c3cccc(-c4cccc5c4-c4ccccc4CO5)c3)cc2)cc1] 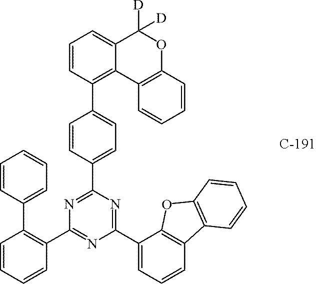 [SMILES: [2H]C1([2H])Oc2ccccc2-c2c(-c3ccc(-c4nc(-c5ccccc5-c5ccccc5)nc(-c5cccc6c5oc5ccccc56)n4)cc3)cccc21] 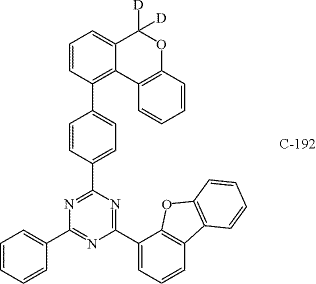 [SMILES: [2H]C1([2H])Oc2ccccc2-c2c(-c3ccc(-c4nc(-c5ccccc5)nc(-c5cccc6c5oc5ccccc56)n4)cc3)cccc21] 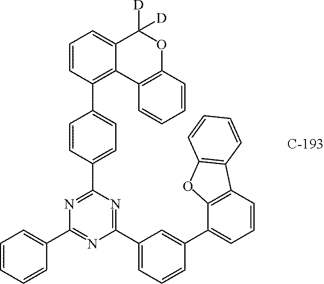 [SMILES: [2H]C1([2H])Oc2ccccc2-c2c(-c3ccc(-c4nc(-c5ccccc5)nc(-c5cccc(-c6cccc7c6oc6ccccc67)c5)n4)cc3)cccc21] 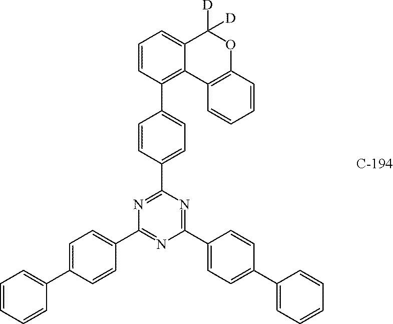 [SMILES: [2H]C1([2H])Oc2ccccc2-c2c(-c3ccc(-c4nc(-c5ccc(-c6ccccc6)cc5)nc(-c5ccc(-c6ccccc6)cc5)n4)cc3)cccc21] 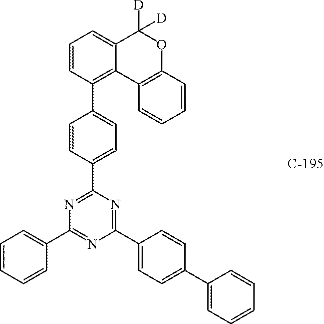 [SMILES: [2H]C1([2H])Oc2ccccc2-c2c(-c3ccc(-c4nc(-c5ccccc5)nc(-c5ccc(-c6ccccc6)cc5)n4)cc3)cccc21] 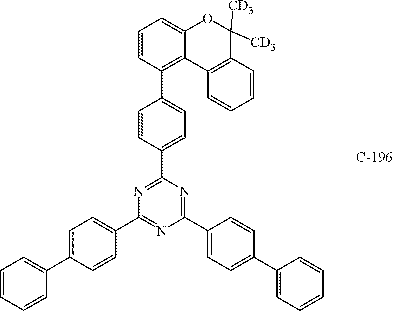 [SMILES: [2H]C([2H])([2H])C1(C([2H])([2H])[2H])Oc2cccc(-c3ccc(-c4nc(-c5ccc(-c6ccccc6)cc5)nc(-c5ccc(-c6ccccc6)cc5)n4)cc3)c2-c2ccccc21] 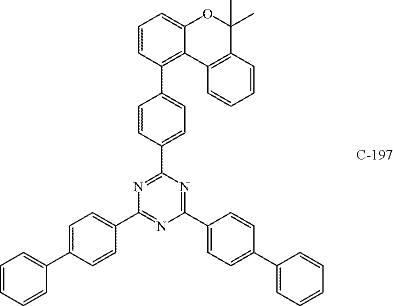 [SMILES: CC1(C)Oc2cccc(-c3ccc(-c4nc(-c5ccc(-c6ccccc6)cc5)nc(-c5ccc(-c6ccccc6)cc5)n4)cc3)c2-c2ccccc21] 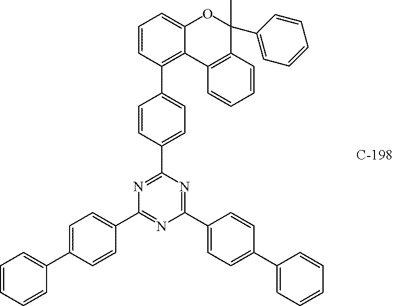 [SMILES: CC1(c2ccccc2)Oc2cccc(-c3ccc(-c4nc(-c5ccc(-c6ccccc6)cc5)nc(-c5ccc(-c6ccccc6)cc5)n4)cc3)c2-c2ccccc21] 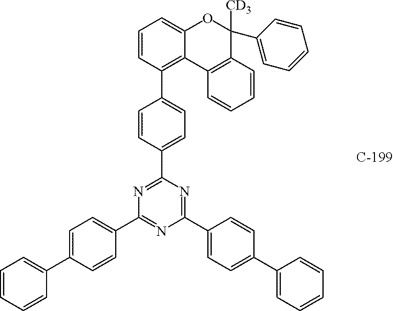 [SMILES: [2H]C([2H])([2H])C1(c2ccccc2)Oc2cccc(-c3ccc(-c4nc(-c5ccc(-c6ccccc6)cc5)nc(-c5ccc(-c6ccccc6)cc5)n4)cc3)c2-c2ccccc21] 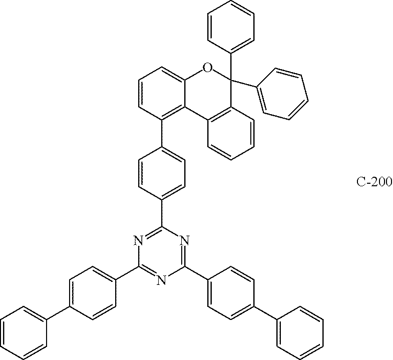 [SMILES: c1ccc(-c2ccc(-c3nc(-c4ccc(-c5ccccc5)cc4)nc(-c4ccc(-c5cccc6c5-c5ccccc5C(c5ccccc5)(c5ccccc5)O6)cc4)n3)cc2)cc1] 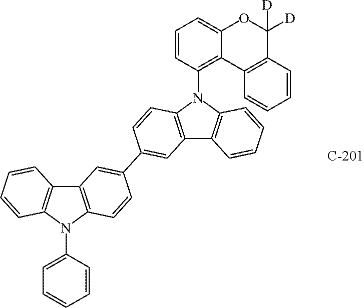 [SMILES: [2H]C1([2H])Oc2cccc(-n3c4ccccc4c4cc(-c5ccc6c(c5)c5ccccc5n6-c5ccccc5)ccc43)c2-c2ccccc21] 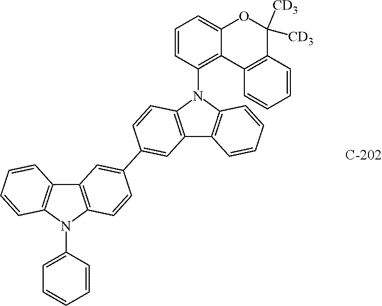 [SMILES: [2H]C([2H])([2H])C1(C([2H])([2H])[2H])Oc2cccc(-n3c4ccccc4c4cc(-c5ccc6c(c5)c5ccccc5n6-c5ccccc5)ccc43)c2-c2ccccc21] 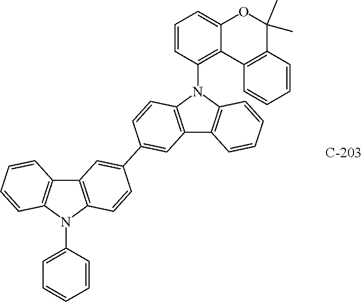 [SMILES: CC1(C)Oc2cccc(-n3c4ccccc4c4cc(-c5ccc6c(c5)c5ccccc5n6-c5ccccc5)ccc43)c2-c2ccccc21] 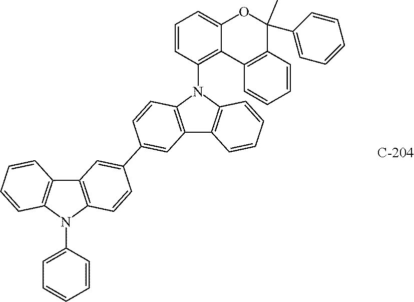 [SMILES: CC1(c2ccccc2)Oc2cccc(-n3c4ccccc4c4cc(-c5ccc6c(c5)c5ccccc5n6-c5ccccc5)ccc43)c2-c2ccccc21] 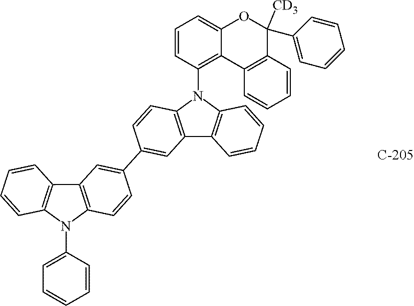 [SMILES: [2H]C([2H])([2H])C1(c2ccccc2)Oc2cccc(-n3c4ccccc4c4cc(-c5ccc6c(c5)c5ccccc5n6-c5ccccc5)ccc43)c2-c2ccccc21] 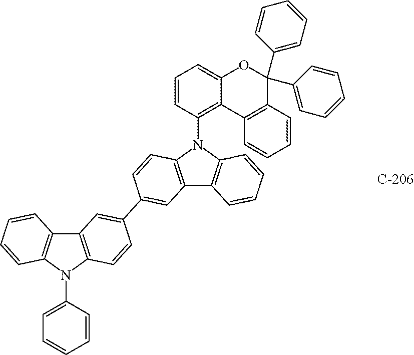 [SMILES: c1ccc(-n2c3ccccc3c3cc(-c4ccc5c(c4)c4ccccc4n5-c4cccc5c4-c4ccccc4C(c4ccccc4)(c4ccccc4)O5)ccc32)cc1] 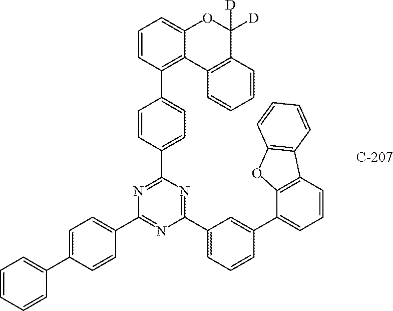 [SMILES: [2H]C1([2H])Oc2cccc(-c3ccc(-c4nc(-c5ccc(-c6ccccc6)cc5)nc(-c5cccc(-c6cccc7c6oc6ccccc67)c5)n4)cc3)c2-c2ccccc21] 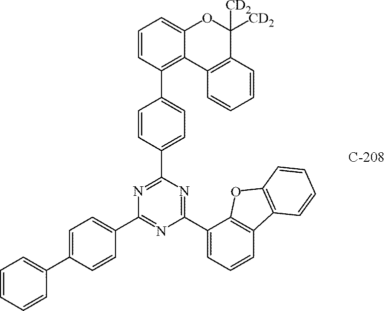 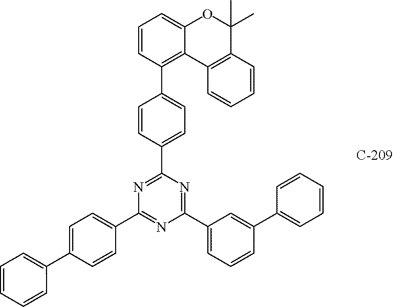 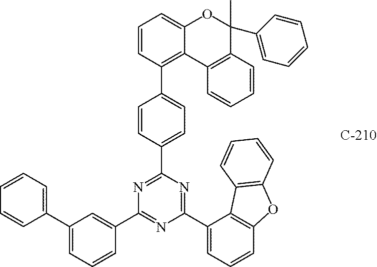 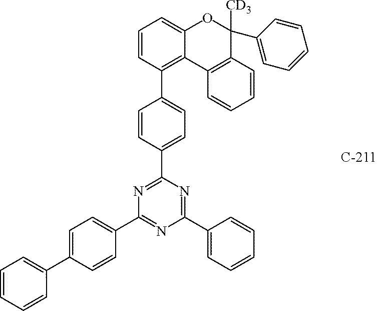 [SMILES: [2H]C([2H])([2H])C1(c2ccccc2)Oc2cccc(-c3ccc(-c4nc(-c5ccccc5)nc(-c5ccc(-c6ccccc6)cc5)n4)cc3)c2-c2ccccc21] 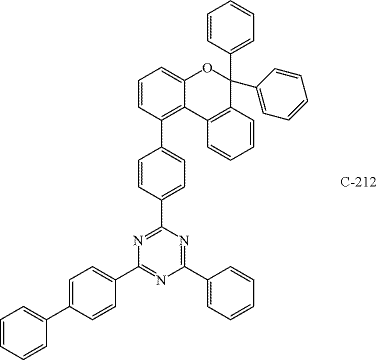 [SMILES: c1ccc(-c2ccc(-c3nc(-c4ccccc4)nc(-c4ccc(-c5cccc6c5-c5ccccc5C(c5ccccc5)(c5ccccc5)O6)cc4)n3)cc2)cc1] 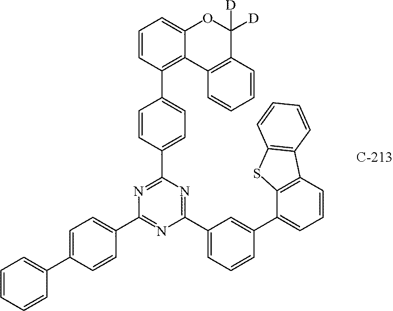 [SMILES: [2H]C1([2H])Oc2cccc(-c3ccc(-c4nc(-c5ccc(-c6ccccc6)cc5)nc(-c5cccc(-c6cccc7c6sc6ccccc67)c5)n4)cc3)c2-c2ccccc21] 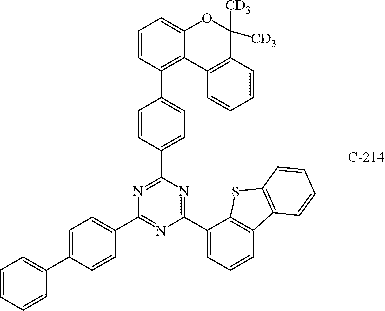 [SMILES: [2H]C([2H])([2H])C1(C([2H])([2H])[2H])Oc2cccc(-c3ccc(-c4nc(-c5ccc(-c6ccccc6)cc5)nc(-c5cccc6c5sc5ccccc56)n4)cc3)c2-c2ccccc21] 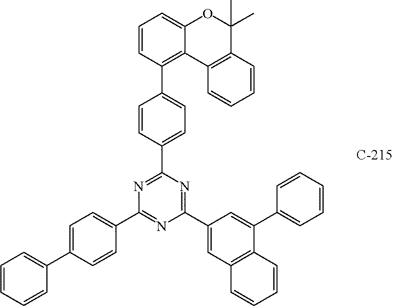 [SMILES: CC1(C)Oc2cccc(-c3ccc(-c4nc(-c5ccc(-c6ccccc6)cc5)nc(-c5cc(-c6ccccc6)c6ccccc6c5)n4)cc3)c2-c2ccccc21] 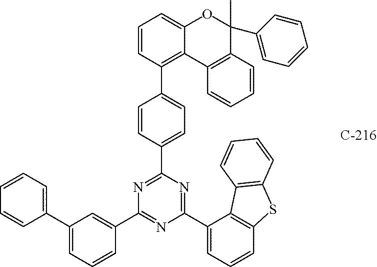 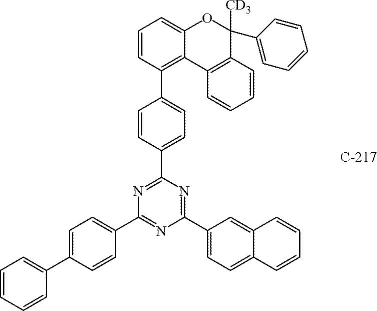 [SMILES: [2H]C([2H])([2H])C1(c2ccccc2)Oc2cccc(-c3ccc(-c4nc(-c5ccc(-c6ccccc6)cc5)nc(-c5ccc6ccccc6c5)n4)cc3)c2-c2ccccc21] 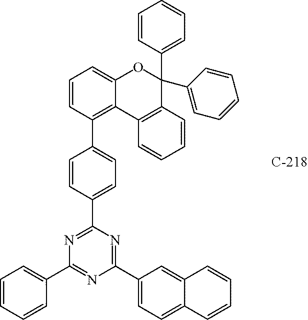 [SMILES: c1ccc(-c2nc(-c3ccc(-c4cccc5c4-c4ccccc4C(c4ccccc4)(c4ccccc4)O5)cc3)nc(-c3ccc4ccccc4c3)n2)cc1] 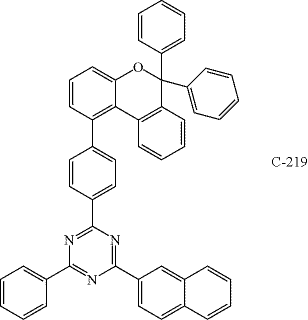 [SMILES: c1ccc(-c2nc(-c3ccc(-c4cccc5c4-c4ccccc4C(c4ccccc4)(c4ccccc4)O5)cc3)nc(-c3ccc4ccccc4c3)n2)cc1] 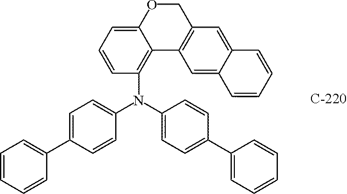 [SMILES: c1ccc(-c2ccc(N(c3ccc(-c4ccccc4)cc3)c3cccc4c3-c3cc5ccccc5cc3CO4)cc2)cc1] 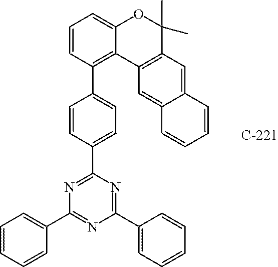 [SMILES: CC1(C)Oc2cccc(-c3ccc(-c4nc(-c5ccccc5)nc(-c5ccccc5)n4)cc3)c2-c2cc3ccccc3cc21] 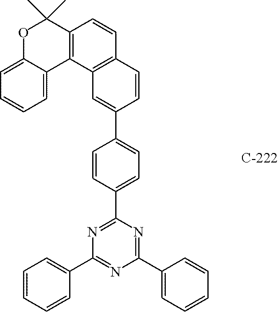 [SMILES: CC1(C)Oc2ccccc2-c2c1ccc1ccc(-c3ccc(-c4nc(-c5ccccc5)nc(-c5ccccc5)n4)cc3)cc21] 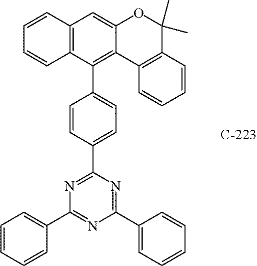 [SMILES: CC1(C)Oc2cc3ccccc3c(-c3ccc(-c4nc(-c5ccccc5)nc(-c5ccccc5)n4)cc3)c2-c2ccccc21] 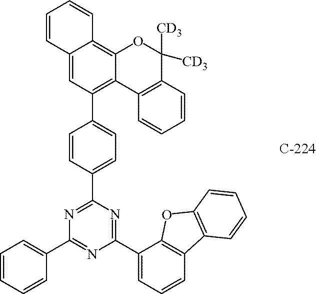 [SMILES: [2H]C([2H])([2H])C1(C([2H])([2H])[2H])Oc2c(c(-c3ccc(-c4nc(-c5ccccc5)nc(-c5cccc6c5oc5ccccc56)n4)cc3)cc3ccccc23)-c2ccccc21] 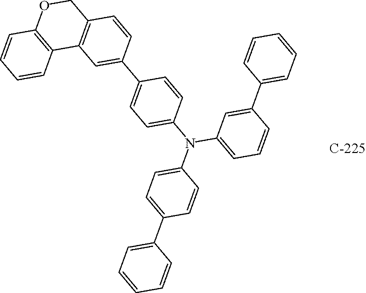 [SMILES: c1ccc(-c2ccc(N(c3ccc(-c4ccc5c(c4)-c4ccccc4OC5)cc3)c3cccc(-c4ccccc4)c3)cc2)cc1] 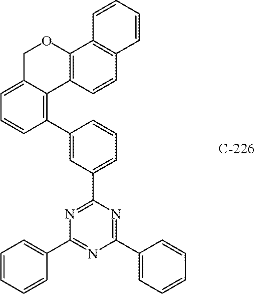 [SMILES: c1ccc(-c2nc(-c3ccccc3)nc(-c3cccc(-c4cccc5c4-c4ccc6ccccc6c4OC5)c3)n2)cc1] 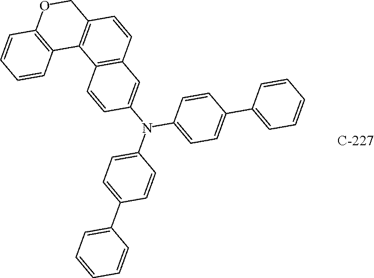 [SMILES: c1ccc(-c2ccc(N(c3ccc(-c4ccccc4)cc3)c3ccc4c5c(ccc4c3)COc3ccccc3-5)cc2)cc1] 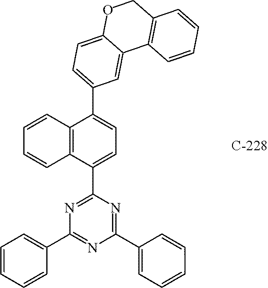 [SMILES: c1ccc(-c2nc(-c3ccccc3)nc(-c3ccc(-c4ccc5c(c4)-c4ccccc4CO5)c4ccccc34)n2)cc1] 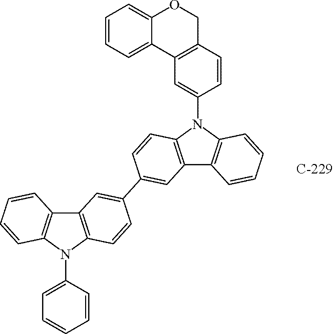 [SMILES: c1ccc(-n2c3ccccc3c3cc(-c4ccc5c(c4)c4ccccc4n5-c4ccc5c(c4)-c4ccccc4OC5)ccc32)cc1] 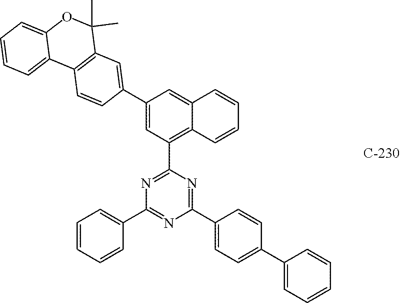 [SMILES: CC1(C)Oc2ccccc2-c2ccc(-c3cc(-c4nc(-c5ccccc5)nc(-c5ccc(-c6ccccc6)cc5)n4)c4ccccc4c3)cc21] 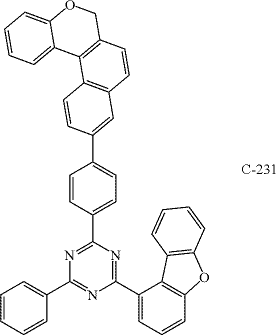 [SMILES: c1ccc(-c2nc(-c3ccc(-c4ccc5c6c(ccc5c4)COc4ccccc4-6)cc3)nc(-c3cccc4oc5ccccc5c34)n2)cc1] 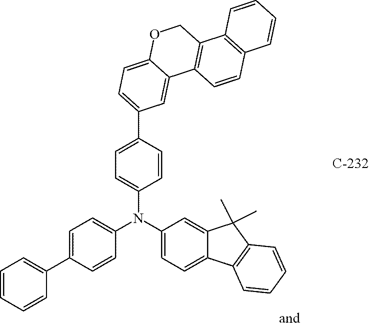 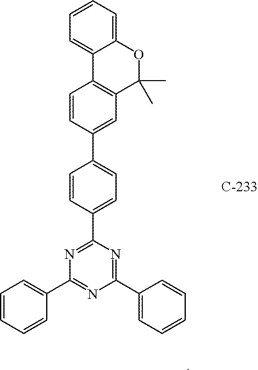
.

6. The plurality of host materials according to claim 1, wherein the compound represented by formula 2 is at least one selected from the group consisting of the following compounds: 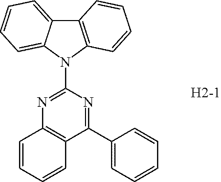 [SMILES: c1ccc(-c2nc(-n3c4ccccc4c4ccccc43)nc3ccccc23)cc1] 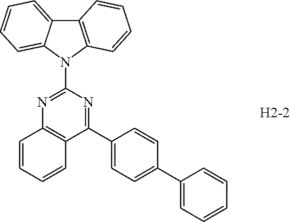 [SMILES: c1ccc(-c2ccc(-c3nc(-n4c5ccccc5c5ccccc54)nc4ccccc34)cc2)cc1] 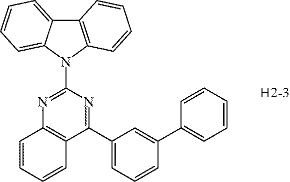 [SMILES: c1ccc(-c2cccc(-c3nc(-n4c5ccccc5c5ccccc54)nc4ccccc34)c2)cc1] 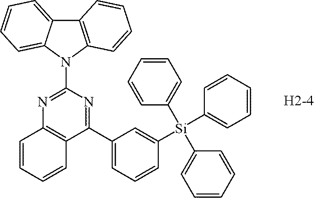 [SMILES: c1ccc([Si](c2ccccc2)(c2ccccc2)c2cccc(-c3nc(-n4c5ccccc5c5ccccc54)nc4ccccc34)c2)cc1] 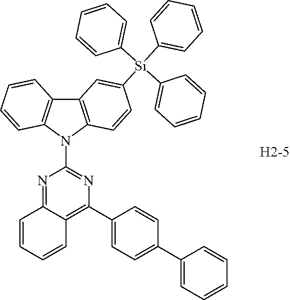 [SMILES: c1ccc(-c2ccc(-c3nc(-n4c5ccccc5c5cc([Si](c6ccccc6)(c6ccccc6)c6ccccc6)ccc54)nc4ccccc34)cc2)cc1] 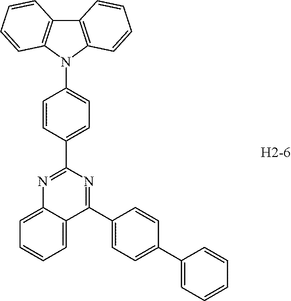 [SMILES: c1ccc(-c2ccc(-c3nc(-c4ccc(-n5c6ccccc6c6ccccc65)cc4)nc4ccccc34)cc2)cc1] 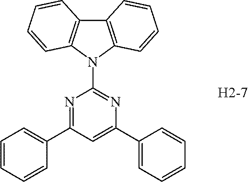 [SMILES: c1ccc(-c2cc(-c3ccccc3)nc(-n3c4ccccc4c4ccccc43)n2)cc1] 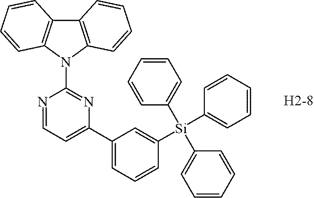 [SMILES: c1ccc([Si](c2ccccc2)(c2ccccc2)c2cccc(-c3ccnc(-n4c5ccccc5c5ccccc54)n3)c2)cc1] 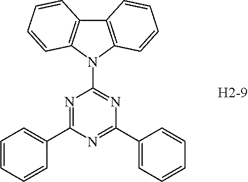 [SMILES: c1ccc(-c2nc(-c3ccccc3)nc(-n3c4ccccc4c4ccccc43)n2)cc1] 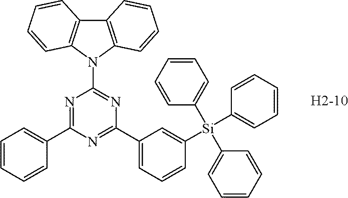 [SMILES: c1ccc(-c2nc(-c3cccc([Si](c4ccccc4)(c4ccccc4)c4ccccc4)c3)nc(-n3c4ccccc4c4ccccc43)n2)cc1] 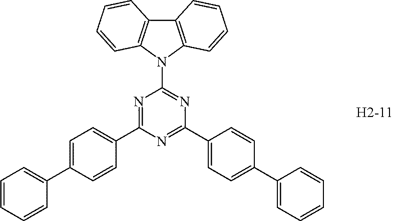 [SMILES: c1ccc(-c2ccc(-c3nc(-c4ccc(-c5ccccc5)cc4)nc(-n4c5ccccc5c5ccccc54)n3)cc2)cc1] 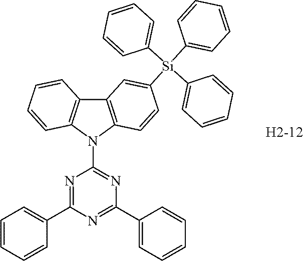 [SMILES: c1ccc(-c2nc(-c3ccccc3)nc(-n3c4ccccc4c4cc([Si](c5ccccc5)(c5ccccc5)c5ccccc5)ccc43)n2)cc1] 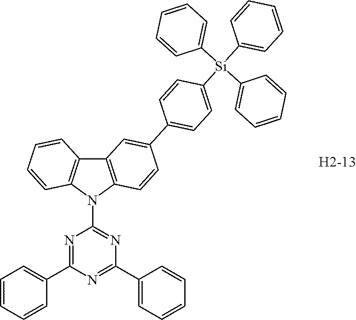 [SMILES: c1ccc(-c2nc(-c3ccccc3)nc(-n3c4ccccc4c4cc(-c5ccc([Si](c6ccccc6)(c6ccccc6)c6ccccc6)cc5)ccc43)n2)cc1] 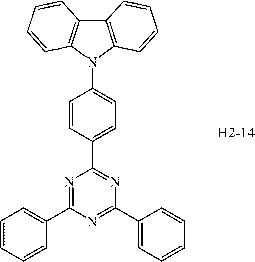 [SMILES: c1ccc(-c2nc(-c3ccccc3)nc(-c3ccc(-n4c5ccccc5c5ccccc54)cc3)n2)cc1] 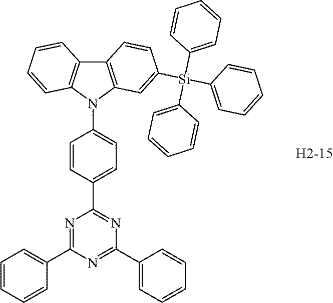 [SMILES: c1ccc(-c2nc(-c3ccccc3)nc(-c3ccc(-n4c5ccccc5c5ccc([Si](c6ccccc6)(c6ccccc6)c6ccccc6)cc54)cc3)n2)cc1] 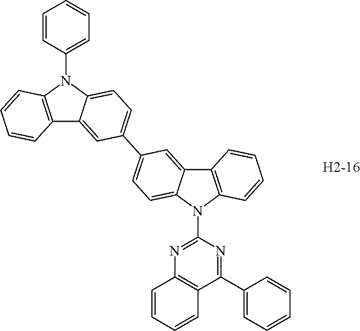 [SMILES: c1ccc(-c2nc(-n3c4ccccc4c4cc(-c5ccc6c(c5)c5ccccc5n6-c5ccccc5)ccc43)nc3ccccc23)cc1] 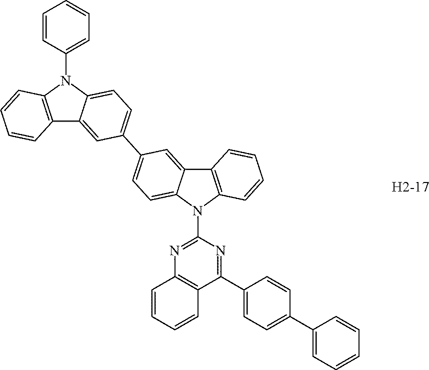 [SMILES: c1ccc(-c2ccc(-c3nc(-n4c5ccccc5c5cc(-c6ccc7c(c6)c6ccccc6n7-c6ccccc6)ccc54)nc4ccccc34)cc2)cc1] 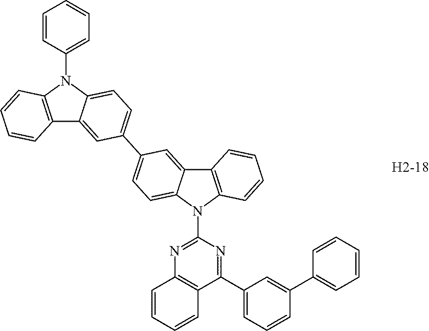 [SMILES: c1ccc(-c2cccc(-c3nc(-n4c5ccccc5c5cc(-c6ccc7c(c6)c6ccccc6n7-c6ccccc6)ccc54)nc4ccccc34)c2)cc1] 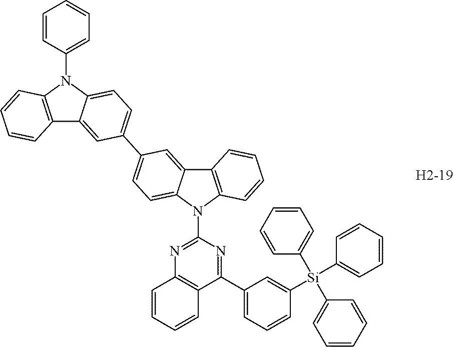 [SMILES: c1ccc(-n2c3ccccc3c3cc(-c4ccc5c(c4)c4ccccc4n5-c4nc(-c5cccc([Si](c6ccccc6)(c6ccccc6)c6ccccc6)c5)c5ccccc5n4)ccc32)cc1] 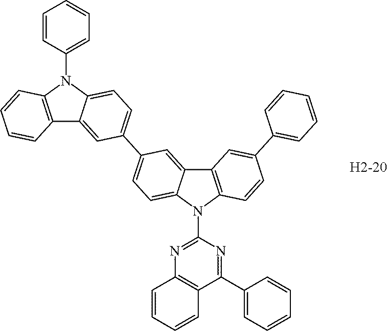 [SMILES: c1ccc(-c2ccc3c(c2)c2cc(-c4ccc5c(c4)c4ccccc4n5-c4ccccc4)ccc2n3-c2nc(-c3ccccc3)c3ccccc3n2)cc1] 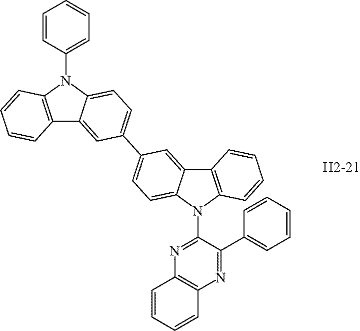 [SMILES: c1ccc(-c2nc3ccccc3nc2-n2c3ccccc3c3cc(-c4ccc5c(c4)c4ccccc4n5-c4ccccc4)ccc32)cc1] 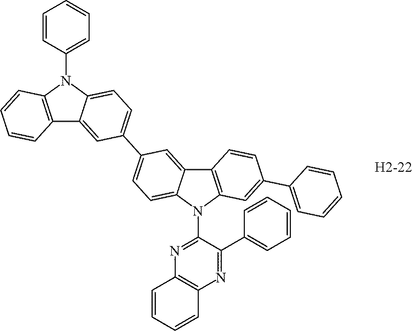 [SMILES: c1ccc(-c2ccc3c4cc(-c5ccc6c(c5)c5ccccc5n6-c5ccccc5)ccc4n(-c4nc5ccccc5nc4-c4ccccc4)c3c2)cc1] 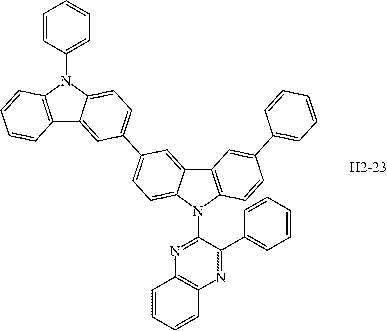 [SMILES: c1ccc(-c2ccc3c(c2)c2cc(-c4ccc5c(c4)c4ccccc4n5-c4ccccc4)ccc2n3-c2nc3ccccc3nc2-c2ccccc2)cc1] 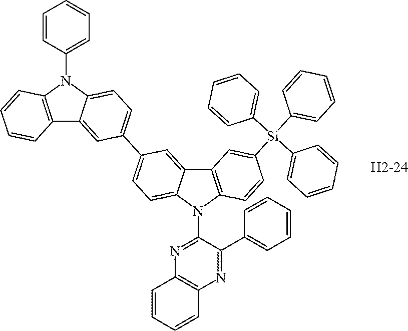 [SMILES: c1ccc(-c2nc3ccccc3nc2-n2c3ccc(-c4ccc5c(c4)c4ccccc4n5-c4ccccc4)cc3c3cc([Si](c4ccccc4)(c4ccccc4)c4ccccc4)ccc32)cc1] 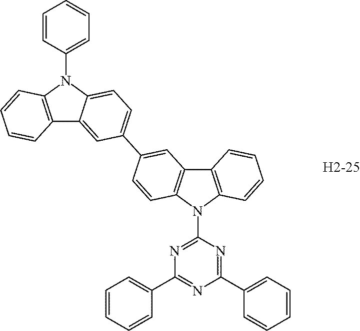 [SMILES: c1ccc(-c2nc(-c3ccccc3)nc(-n3c4ccccc4c4cc(-c5ccc6c(c5)c5ccccc5n6-c5ccccc5)ccc43)n2)cc1] 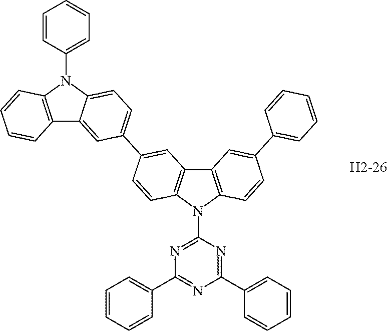 [SMILES: c1ccc(-c2ccc3c(c2)c2cc(-c4ccc5c(c4)c4ccccc4n5-c4ccccc4)ccc2n3-c2nc(-c3ccccc3)nc(-c3ccccc3)n2)cc1] 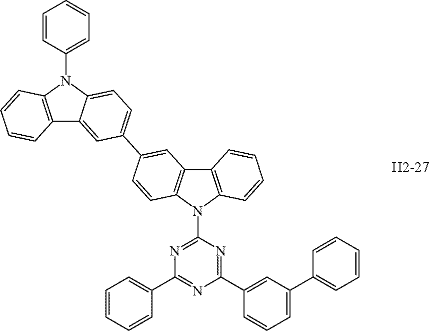 [SMILES: c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1] 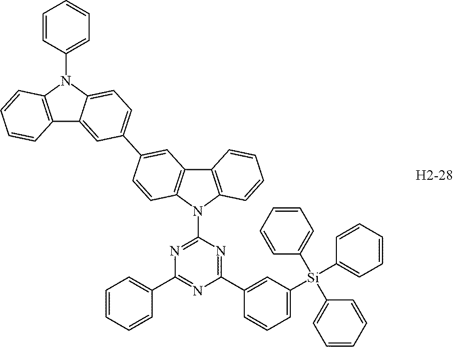 [SMILES: c1ccc(-c2nc(-c3cccc([Si](c4ccccc4)(c4ccccc4)c4ccccc4)c3)nc(-n3c4ccccc4c4cc(-c5ccc6c(c5)c5ccccc5n6-c5ccccc5)ccc43)n2)cc1] 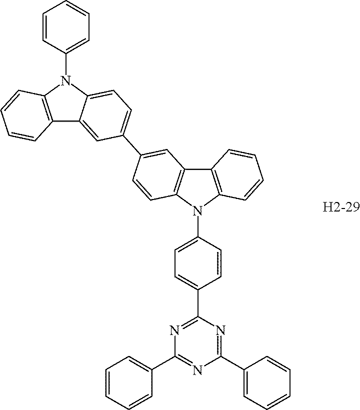 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1] 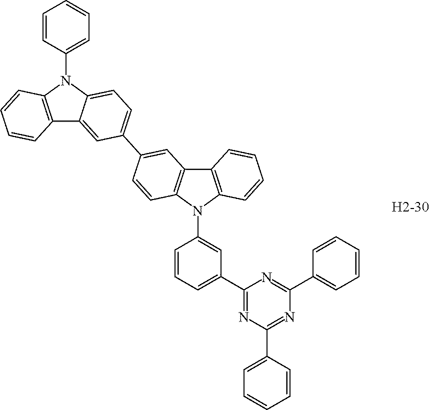 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1] 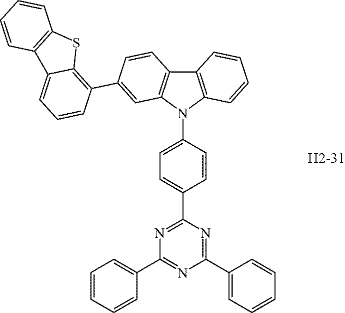 [SMILES: c1ccc(-c2nc(-c3ccccc3)nc(-c3ccc(-n4c5ccccc5c5ccc(-c6cccc7c6sc6ccccc67)cc54)cc3)n2)cc1] 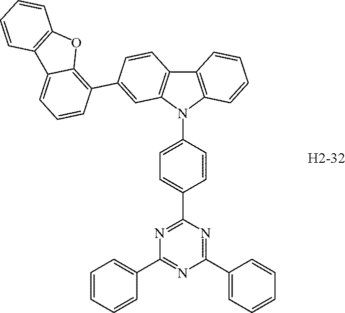 [SMILES: c1ccc(-c2nc(-c3ccccc3)nc(-c3ccc(-n4c5ccccc5c5ccc(-c6cccc7c6oc6ccccc67)cc54)cc3)n2)cc1] 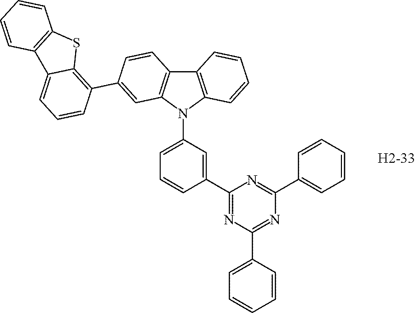 [SMILES: c1ccc(-c2nc(-c3ccccc3)nc(-c3cccc(-n4c5ccccc5c5ccc(-c6cccc7c6sc6ccccc67)cc54)c3)n2)cc1] 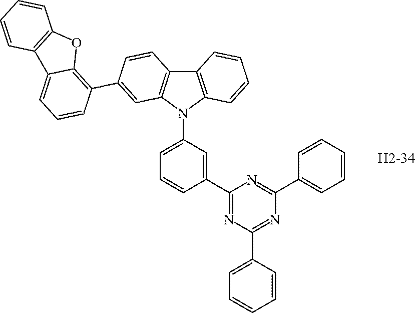 [SMILES: c1ccc(-c2nc(-c3ccccc3)nc(-c3cccc(-n4c5ccccc5c5ccc(-c6cccc7c6oc6ccccc67)cc54)c3)n2)cc1] 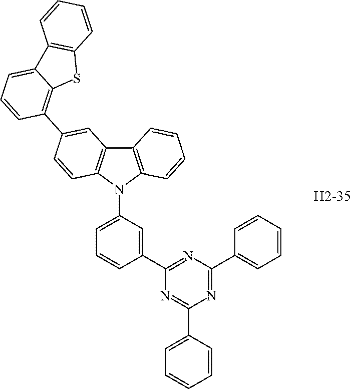 [SMILES: c1ccc(-c2nc(-c3ccccc3)nc(-c3cccc(-n4c5ccccc5c5cc(-c6cccc7c6sc6ccccc67)ccc54)c3)n2)cc1] 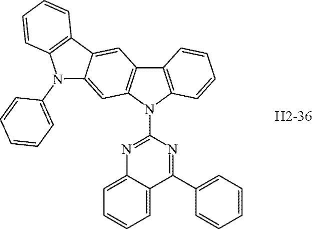 [SMILES: c1ccc(-c2nc(-n3c4ccccc4c4cc5c6ccccc6n(-c6ccccc6)c5cc43)nc3ccccc23)cc1] 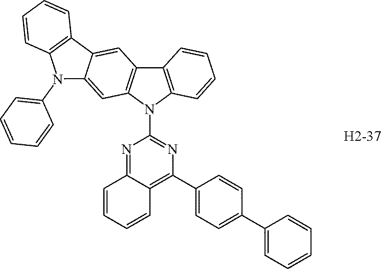 [SMILES: c1ccc(-c2ccc(-c3nc(-n4c5ccccc5c5cc6c7ccccc7n(-c7ccccc7)c6cc54)nc4ccccc34)cc2)cc1] 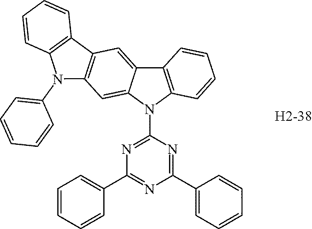 [SMILES: c1ccc(-c2nc(-c3ccccc3)nc(-n3c4ccccc4c4cc5c6ccccc6n(-c6ccccc6)c5cc43)n2)cc1] 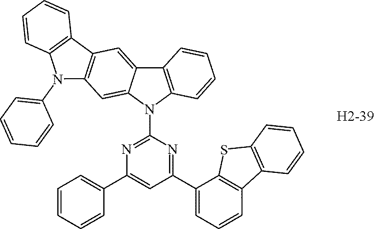 [SMILES: c1ccc(-c2cc(-c3cccc4c3sc3ccccc34)nc(-n3c4ccccc4c4cc5c6ccccc6n(-c6ccccc6)c5cc43)n2)cc1] 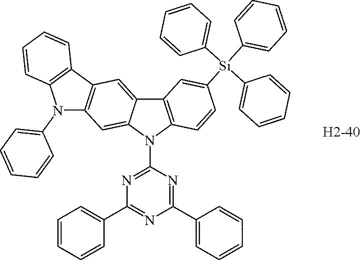 [SMILES: c1ccc(-c2nc(-c3ccccc3)nc(-n3c4ccc([Si](c5ccccc5)(c5ccccc5)c5ccccc5)cc4c4cc5c6ccccc6n(-c6ccccc6)c5cc43)n2)cc1] 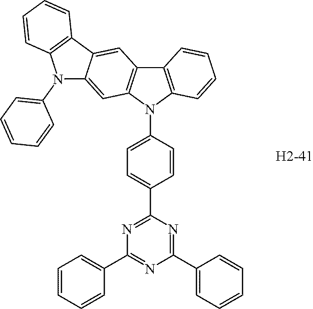 [SMILES: c1ccc(-c2nc(-c3ccccc3)nc(-c3ccc(-n4c5ccccc5c5cc6c7ccccc7n(-c7ccccc7)c6cc54)cc3)n2)cc1] 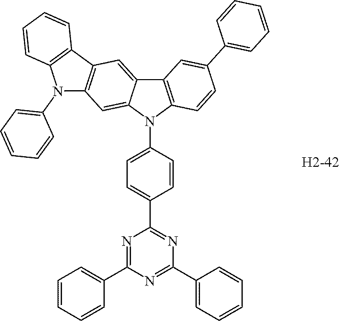 [SMILES: c1ccc(-c2ccc3c(c2)c2cc4c5ccccc5n(-c5ccccc5)c4cc2n3-c2ccc(-c3nc(-c4ccccc4)nc(-c4ccccc4)n3)cc2)cc1] 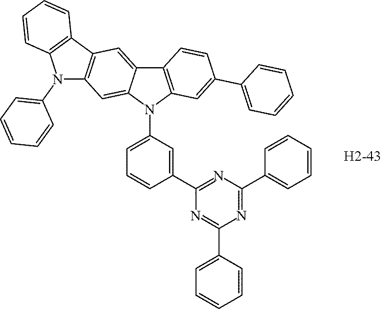 [SMILES: c1ccc(-c2ccc3c4cc5c6ccccc6n(-c6ccccc6)c5cc4n(-c4cccc(-c5nc(-c6ccccc6)nc(-c6ccccc6)n5)c4)c3c2)cc1] 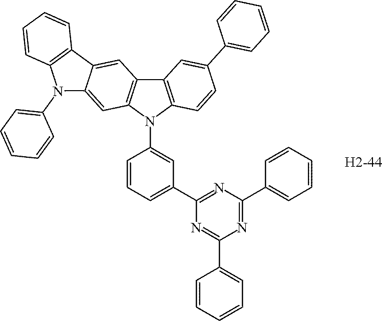 [SMILES: c1ccc(-c2ccc3c(c2)c2cc4c5ccccc5n(-c5ccccc5)c4cc2n3-c2cccc(-c3nc(-c4ccccc4)nc(-c4ccccc4)n3)c2)cc1] 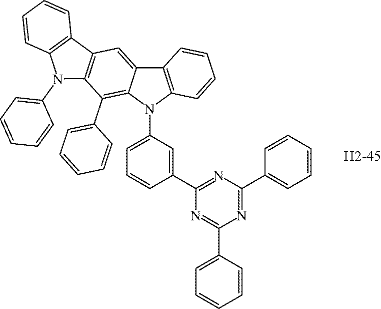 [SMILES: c1ccc(-c2nc(-c3ccccc3)nc(-c3cccc(-n4c5ccccc5c5cc6c7ccccc7n(-c7ccccc7)c6c(-c6ccccc6)c54)c3)n2)cc1] 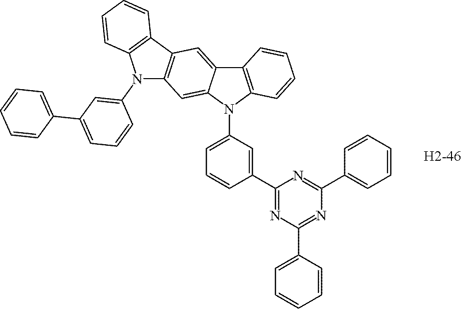 [SMILES: c1ccc(-c2cccc(-n3c4ccccc4c4cc5c6ccccc6n(-c6cccc(-c7nc(-c8ccccc8)nc(-c8ccccc8)n7)c6)c5cc43)c2)cc1] 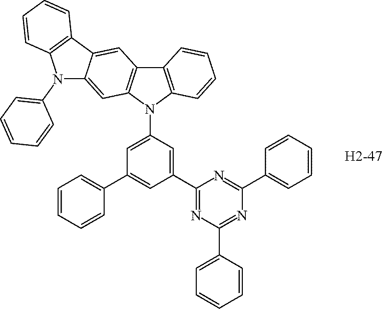 [SMILES: c1ccc(-c2cc(-c3nc(-c4ccccc4)nc(-c4ccccc4)n3)cc(-n3c4ccccc4c4cc5c6ccccc6n(-c6ccccc6)c5cc43)c2)cc1] 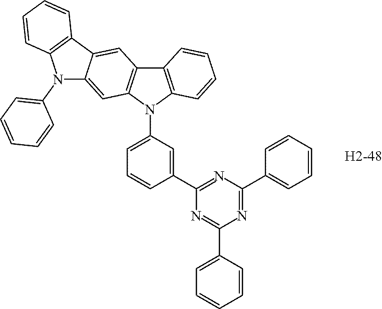 [SMILES: c1ccc(-c2nc(-c3ccccc3)nc(-c3cccc(-n4c5ccccc5c5cc6c7ccccc7n(-c7ccccc7)c6cc54)c3)n2)cc1] 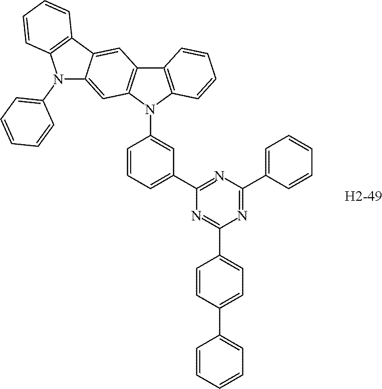 [SMILES: c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1] 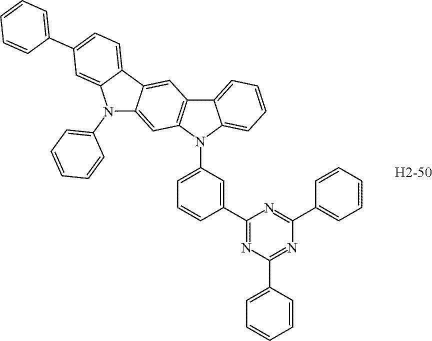 [SMILES: c1ccc(-c2ccc3c4cc5c6ccccc6n(-c6cccc(-c7nc(-c8ccccc8)nc(-c8ccccc8)n7)c6)c5cc4n(-c4ccccc4)c3c2)cc1] 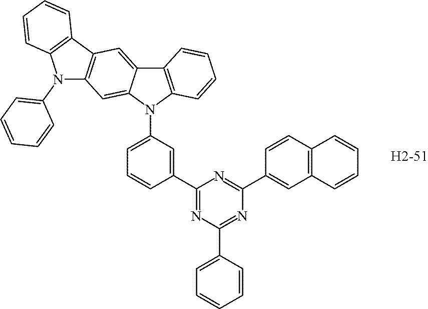 [SMILES: c1ccc(-c2nc(-c3cccc(-n4c5ccccc5c5cc6c7ccccc7n(-c7ccccc7)c6cc54)c3)nc(-c3ccc4ccccc4c3)n2)cc1] 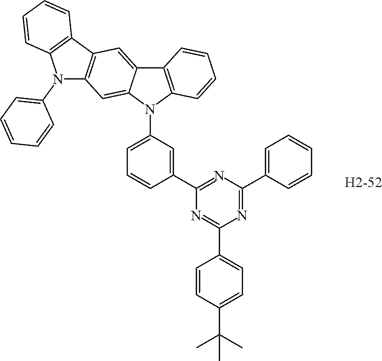 [SMILES: CC(C)(C)c1ccc(-c2nc(-c3ccccc3)nc(-c3cccc(-n4c5ccccc5c5cc6c7ccccc7n(-c7ccccc7)c6cc54)c3)n2)cc1] 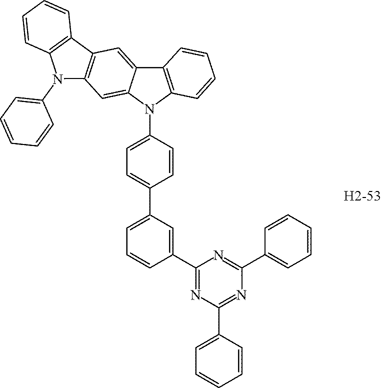 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1] 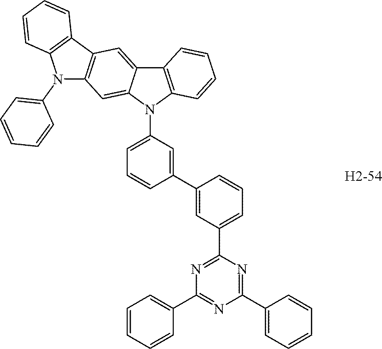 [SMILES: c1ccc(-c2nc(-c3ccccc3)nc(-c3cccc(-c4cccc(-n5c6ccccc6c6cc7c8ccccc8n(-c8ccccc8)c7cc65)c4)c3)n2)cc1] 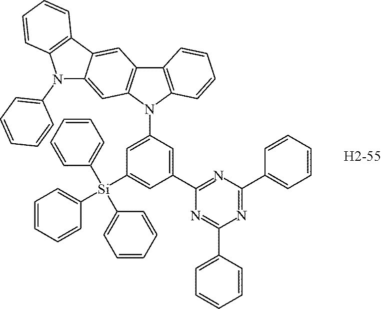 [SMILES: c1ccc(-c2nc(-c3ccccc3)nc(-c3cc(-n4c5ccccc5c5cc6c7ccccc7n(-c7ccccc7)c6cc54)cc([Si](c4ccccc4)(c4ccccc4)c4ccccc4)c3)n2)cc1] 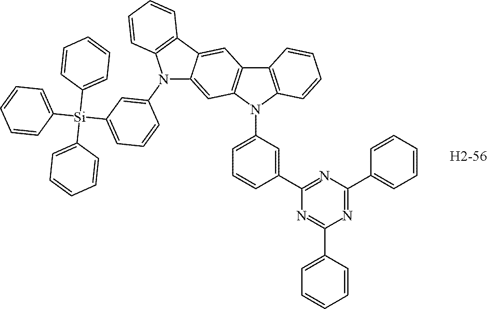 [SMILES: c1ccc(-c2nc(-c3ccccc3)nc(-c3cccc(-n4c5ccccc5c5cc6c7ccccc7n(-c7cccc([Si](c8ccccc8)(c8ccccc8)c8ccccc8)c7)c6cc54)c3)n2)cc1] 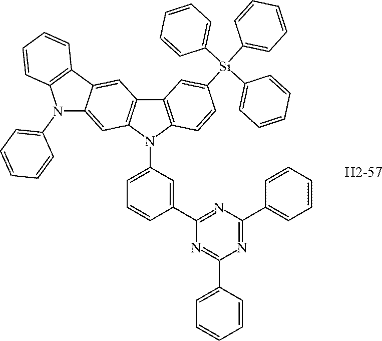 [SMILES: c1ccc(-c2nc(-c3ccccc3)nc(-c3cccc(-n4c5ccc([Si](c6ccccc6)(c6ccccc6)c6ccccc6)cc5c5cc6c7ccccc7n(-c7ccccc7)c6cc54)c3)n2)cc1] 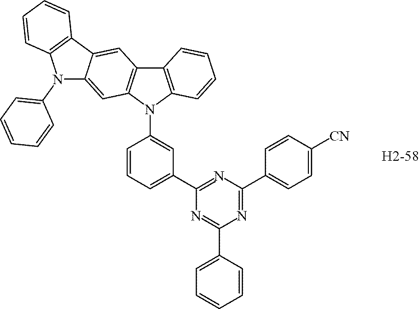 [SMILES: N#Cc1ccc(-c2nc(-c3ccccc3)nc(-c3cccc(-n4c5ccccc5c5cc6c7ccccc7n(-c7ccccc7)c6cc54)c3)n2)cc1] 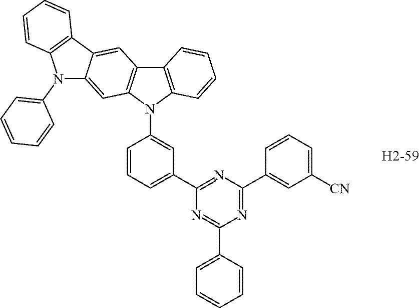 [SMILES: N#Cc1cccc(-c2nc(-c3ccccc3)nc(-c3cccc(-n4c5ccccc5c5cc6c7ccccc7n(-c7ccccc7)c6cc54)c3)n2)c1] 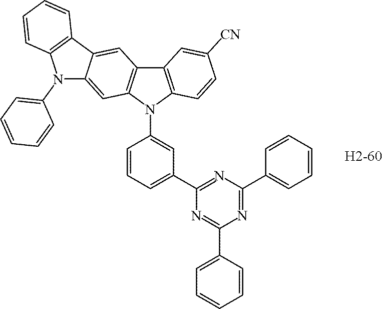 [SMILES: N#Cc1ccc2c(c1)c1cc3c4ccccc4n(-c4ccccc4)c3cc1n2-c1cccc(-c2nc(-c3ccccc3)nc(-c3ccccc3)n2)c1] 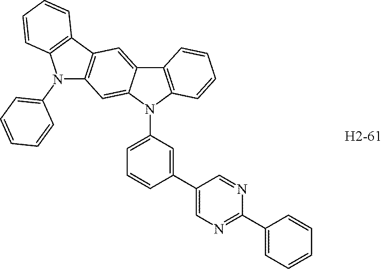 [SMILES: c1ccc(-c2ncc(-c3cccc(-n4c5ccccc5c5cc6c7ccccc7n(-c7ccccc7)c6cc54)c3)cn2)cc1] 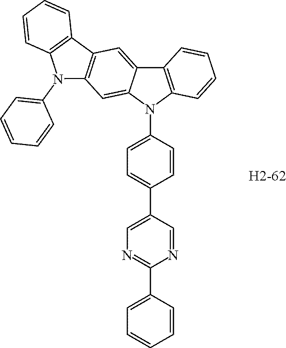 [SMILES: c1ccc(-c2ncc(-c3ccc(-n4c5ccccc5c5cc6c7ccccc7n(-c7ccccc7)c6cc54)cc3)cn2)cc1] 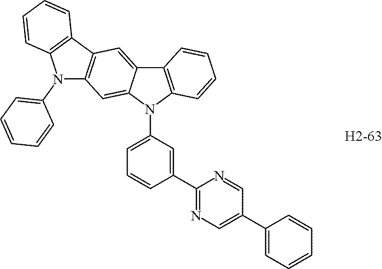 [SMILES: c1ccc(-c2cnc(-c3cccc(-n4c5ccccc5c5cc6c7ccccc7n(-c7ccccc7)c6cc54)c3)nc2)cc1] 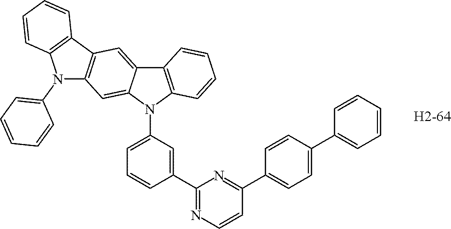 [SMILES: c1ccc(-c2ccc(-c3ccnc(-c4cccc(-n5c6ccccc6c6cc7c8ccccc8n(-c8ccccc8)c7cc65)c4)n3)cc2)cc1] 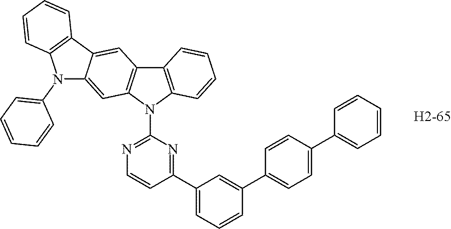 [SMILES: c1ccc(-c2ccc(-c3cccc(-c4ccnc(-n5c6ccccc6c6cc7c8ccccc8n(-c8ccccc8)c7cc65)n4)c3)cc2)cc1] 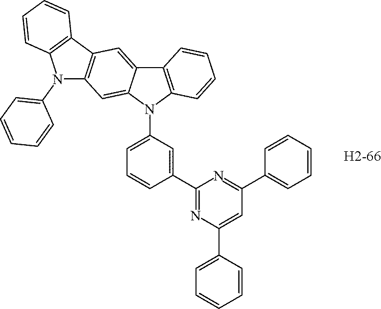 [SMILES: c1ccc(-c2cc(-c3ccccc3)nc(-c3cccc(-n4c5ccccc5c5cc6c7ccccc7n(-c7ccccc7)c6cc54)c3)n2)cc1] 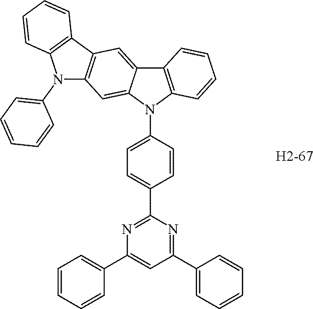 [SMILES: c1ccc(-c2cc(-c3ccccc3)nc(-c3ccc(-n4c5ccccc5c5cc6c7ccccc7n(-c7ccccc7)c6cc54)cc3)n2)cc1] 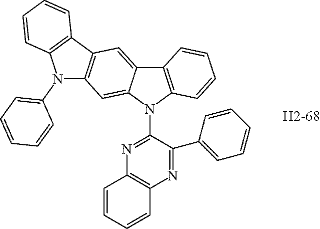 [SMILES: c1ccc(-c2nc3ccccc3nc2-n2c3ccccc3c3cc4c5ccccc5n(-c5ccccc5)c4cc32)cc1] 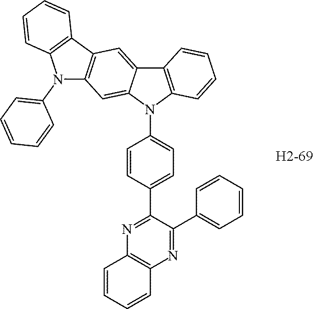 [SMILES: c1ccc(-c2nc3ccccc3nc2-c2ccc(-n3c4ccccc4c4cc5c6ccccc6n(-c6ccccc6)c5cc43)cc2)cc1] 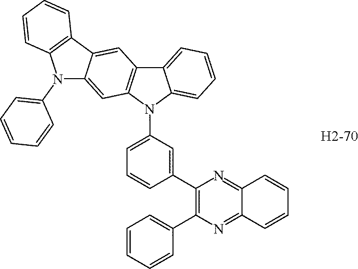 [SMILES: c1ccc(-c2nc3ccccc3nc2-c2cccc(-n3c4ccccc4c4cc5c6ccccc6n(-c6ccccc6)c5cc43)c2)cc1] 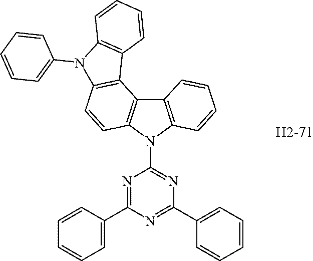 [SMILES: c1ccc(-c2nc(-c3ccccc3)nc(-n3c4ccccc4c4c5c6ccccc6n(-c6ccccc6)c5ccc43)n2)cc1] 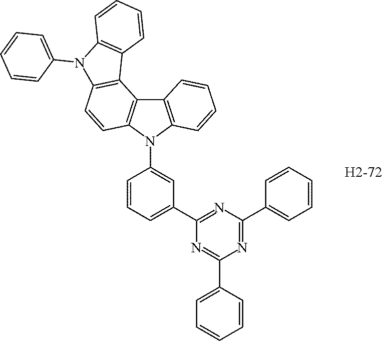 [SMILES: c1ccc(-c2nc(-c3ccccc3)nc(-c3cccc(-n4c5ccccc5c5c6c7ccccc7n(-c7ccccc7)c6ccc54)c3)n2)cc1] 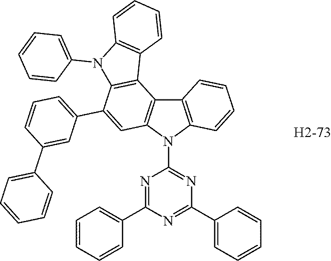 [SMILES: c1ccc(-c2cccc(-c3cc4c(c5ccccc5n4-c4nc(-c5ccccc5)nc(-c5ccccc5)n4)c4c5ccccc5n(-c5ccccc5)c34)c2)cc1] 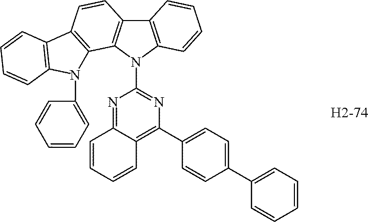 [SMILES: c1ccc(-c2ccc(-c3nc(-n4c5ccccc5c5ccc6c7ccccc7n(-c7ccccc7)c6c54)nc4ccccc34)cc2)cc1] 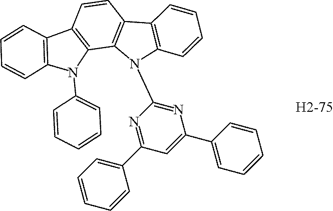 [SMILES: c1ccc(-c2cc(-c3ccccc3)nc(-n3c4ccccc4c4ccc5c6ccccc6n(-c6ccccc6)c5c43)n2)cc1] 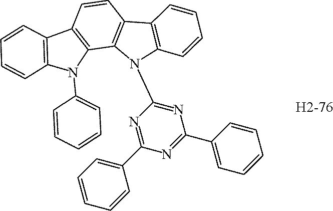 [SMILES: c1ccc(-c2nc(-c3ccccc3)nc(-n3c4ccccc4c4ccc5c6ccccc6n(-c6ccccc6)c5c43)n2)cc1] 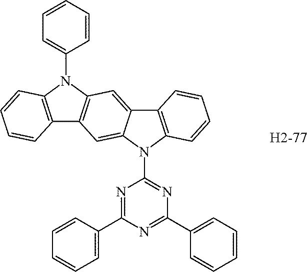 [SMILES: c1ccc(-c2nc(-c3ccccc3)nc(-n3c4ccccc4c4cc5c(cc43)c3ccccc3n5-c3ccccc3)n2)cc1] 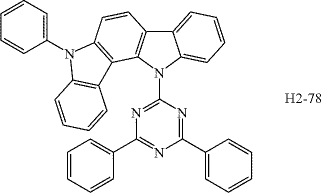 [SMILES: c1ccc(-c2nc(-c3ccccc3)nc(-n3c4ccccc4c4ccc5c(c6ccccc6n5-c5ccccc5)c43)n2)cc1] 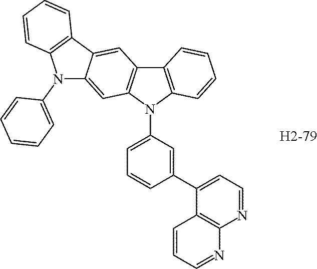 [SMILES: c1ccc(-n2c3ccccc3c3cc4c5ccccc5n(-c5cccc(-c6ccnc7ncccc67)c5)c4cc32)cc1] 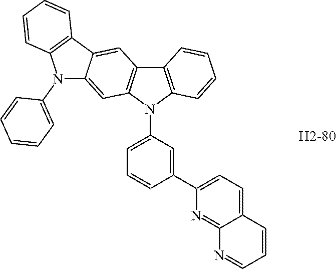 [SMILES: c1ccc(-n2c3ccccc3c3cc4c5ccccc5n(-c5cccc(-c6ccc7cccnc7n6)c5)c4cc32)cc1] 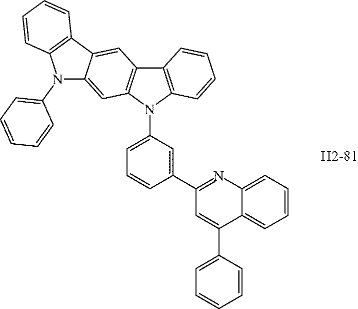 [SMILES: c1ccc(-c2cc(-c3cccc(-n4c5ccccc5c5cc6c7ccccc7n(-c7ccccc7)c6cc54)c3)nc3ccccc23)cc1] 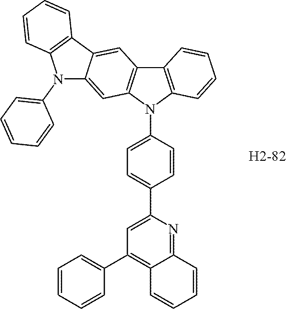 [SMILES: c1ccc(-c2cc(-c3ccc(-n4c5ccccc5c5cc6c7ccccc7n(-c7ccccc7)c6cc54)cc3)nc3ccccc23)cc1] 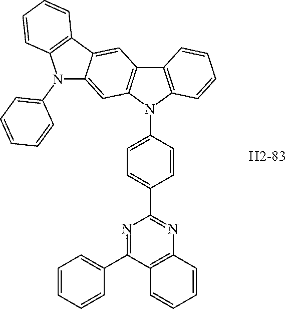 [SMILES: c1ccc(-c2nc(-c3ccc(-n4c5ccccc5c5cc6c7ccccc7n(-c7ccccc7)c6cc54)cc3)nc3ccccc23)cc1] 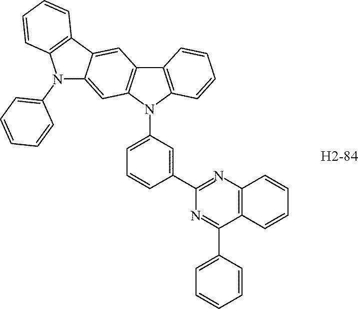 [SMILES: c1ccc(-c2nc(-c3cccc(-n4c5ccccc5c5cc6c7ccccc7n(-c7ccccc7)c6cc54)c3)nc3ccccc23)cc1] 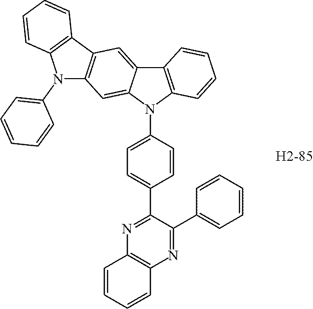 [SMILES: c1ccc(-c2nc3ccccc3nc2-c2ccc(-n3c4ccccc4c4cc5c6ccccc6n(-c6ccccc6)c5cc43)cc2)cc1] 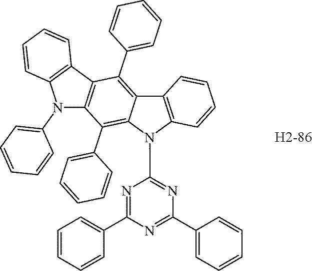 [SMILES: c1ccc(-c2nc(-c3ccccc3)nc(-n3c4ccccc4c4c(-c5ccccc5)c5c6ccccc6n(-c6ccccc6)c5c(-c5ccccc5)c43)n2)cc1] 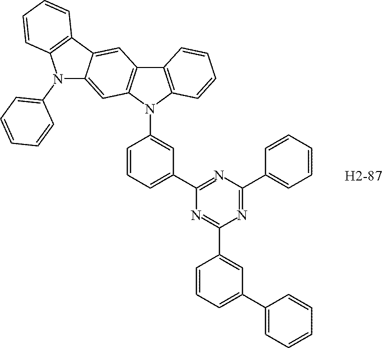 [SMILES: c1ccc(-c2cccc(-c3nc(-c4ccccc4)nc(-c4cccc(-n5c6ccccc6c6cc7c8ccccc8n(-c8ccccc8)c7cc65)c4)n3)c2)cc1] 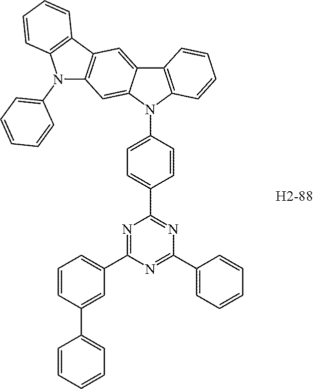 [SMILES: c1ccc(-c2cccc(-c3nc(-c4ccccc4)nc(-c4ccc(-n5c6ccccc6c6cc7c8ccccc8n(-c8ccccc8)c7cc65)cc4)n3)c2)cc1] 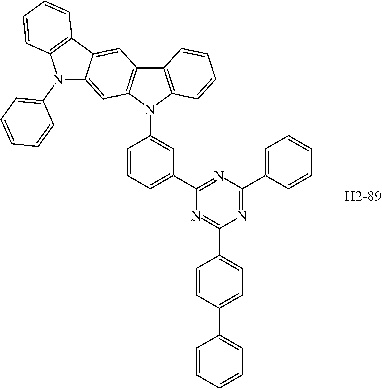 [SMILES: c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1] 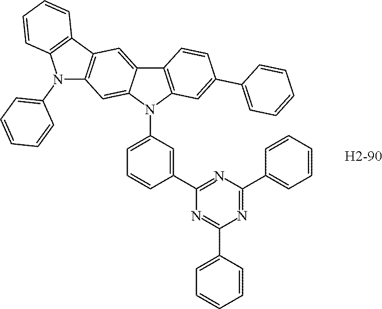 [SMILES: c1ccc(-c2ccc3c4cc5c6ccccc6n(-c6ccccc6)c5cc4n(-c4cccc(-c5nc(-c6ccccc6)nc(-c6ccccc6)n5)c4)c3c2)cc1] 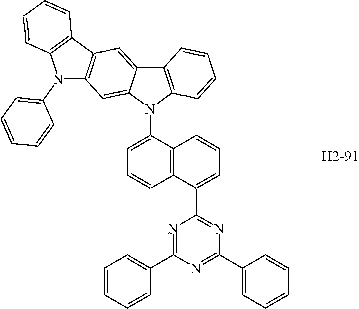 [SMILES: c1ccc(-c2nc(-c3ccccc3)nc(-c3cccc4c(-n5c6ccccc6c6cc7c8ccccc8n(-c8ccccc8)c7cc65)cccc34)n2)cc1] 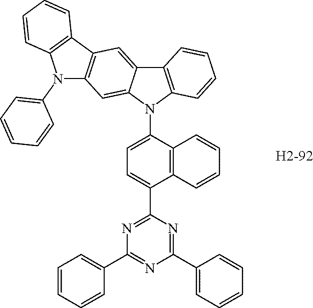 [SMILES: c1ccc(-c2nc(-c3ccccc3)nc(-c3ccc(-n4c5ccccc5c5cc6c7ccccc7n(-c7ccccc7)c6cc54)c4ccccc34)n2)cc1] 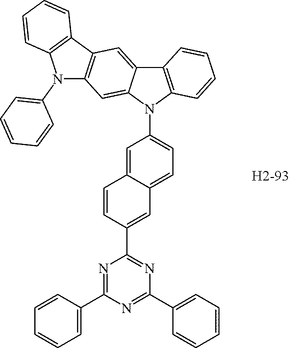 [SMILES: c1ccc(-c2nc(-c3ccccc3)nc(-c3ccc4cc(-n5c6ccccc6c6cc7c8ccccc8n(-c8ccccc8)c7cc65)ccc4c3)n2)cc1] 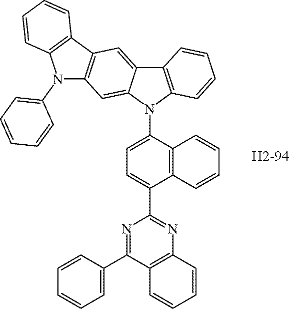 [SMILES: c1ccc(-c2nc(-c3ccc(-n4c5ccccc5c5cc6c7ccccc7n(-c7ccccc7)c6cc54)c4ccccc34)nc3ccccc23)cc1] 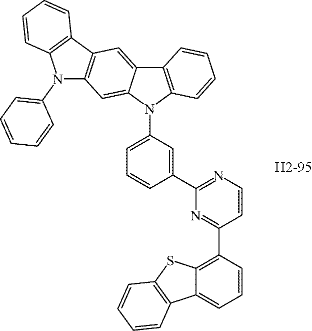 [SMILES: c1ccc(-n2c3ccccc3c3cc4c5ccccc5n(-c5cccc(-c6nccc(-c7cccc8c7sc7ccccc78)n6)c5)c4cc32)cc1] 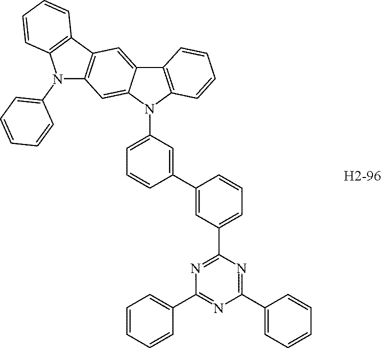 [SMILES: c1ccc(-c2nc(-c3ccccc3)nc(-c3cccc(-c4cccc(-n5c6ccccc6c6cc7c8ccccc8n(-c8ccccc8)c7cc65)c4)c3)n2)cc1] 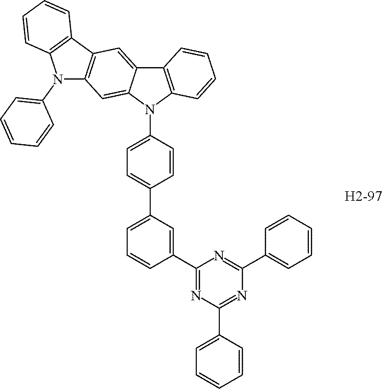 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1] 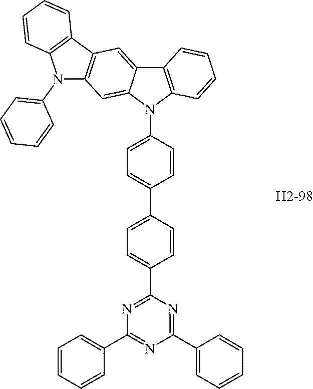 [SMILES: c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1] 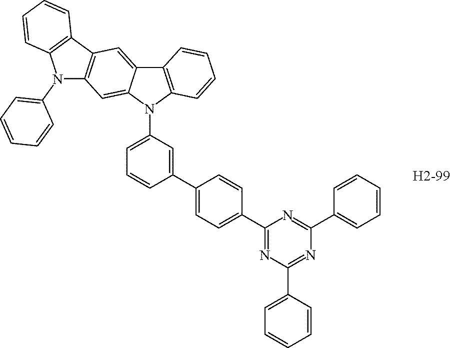 [SMILES: c1ccc(-c2nc(-c3ccccc3)nc(-c3ccc(-c4cccc(-n5c6ccccc6c6cc7c8ccccc8n(-c8ccccc8)c7cc65)c4)cc3)n2)cc1] 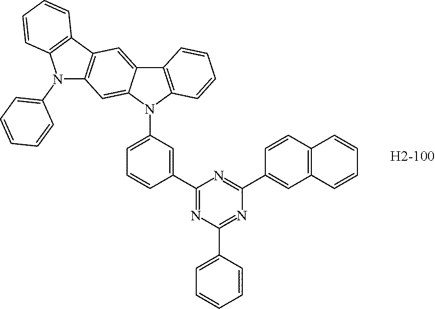 [SMILES: c1ccc(-c2nc(-c3cccc(-n4c5ccccc5c5cc6c7ccccc7n(-c7ccccc7)c6cc54)c3)nc(-c3ccc4ccccc4c3)n2)cc1] 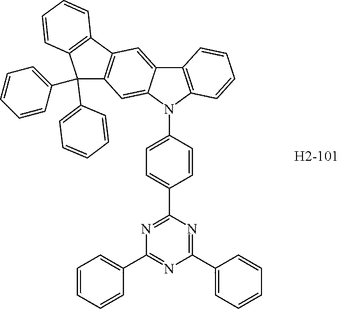 [SMILES: c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1] 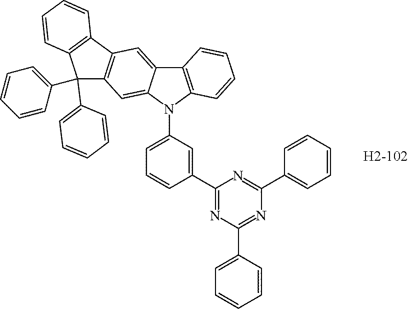 [SMILES: c1ccc(-c2nc(-c3ccccc3)nc(-c3cccc(-n4c5ccccc5c5cc6c(cc54)C(c4ccccc4)(c4ccccc4)c4ccccc4-6)c3)n2)cc1] 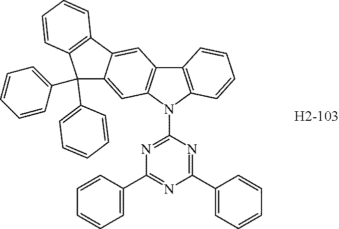 [SMILES: c1ccc(-c2nc(-c3ccccc3)nc(-n3c4ccccc4c4cc5c(cc43)C(c3ccccc3)(c3ccccc3)c3ccccc3-5)n2)cc1] 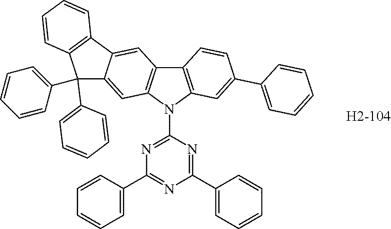 [SMILES: c1ccc(-c2ccc3c4cc5c(cc4n(-c4nc(-c6ccccc6)nc(-c6ccccc6)n4)c3c2)C(c2ccccc2)(c2ccccc2)c2ccccc2-5)cc1] 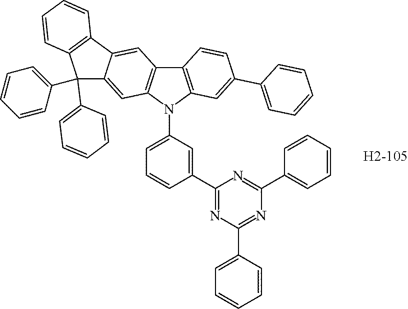 [SMILES: c1ccc(-c2ccc3c4cc5c(cc4n(-c4cccc(-c6nc(-c7ccccc7)nc(-c7ccccc7)n6)c4)c3c2)C(c2ccccc2)(c2ccccc2)c2ccccc2-5)cc1] 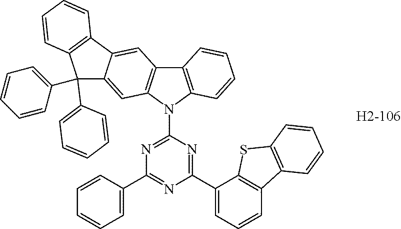 [SMILES: c1ccc(-c2nc(-c3cccc4c3sc3ccccc34)nc(-n3c4ccccc4c4cc5c(cc43)C(c3ccccc3)(c3ccccc3)c3ccccc3-5)n2)cc1] 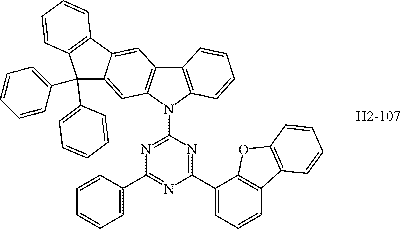 [SMILES: c1ccc(-c2nc(-c3cccc4c3oc3ccccc34)nc(-n3c4ccccc4c4cc5c(cc43)C(c3ccccc3)(c3ccccc3)c3ccccc3-5)n2)cc1] 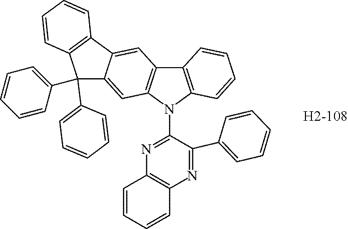 [SMILES: c1ccc(-c2nc3ccccc3nc2-n2c3ccccc3c3cc4c(cc32)C(c2ccccc2)(c2ccccc2)c2ccccc2-4)cc1] 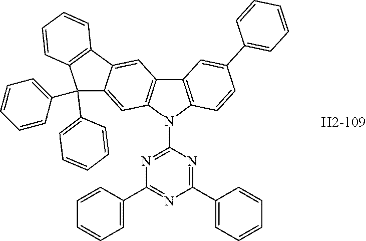 [SMILES: c1ccc(-c2ccc3c(c2)c2cc4c(cc2n3-c2nc(-c3ccccc3)nc(-c3ccccc3)n2)C(c2ccccc2)(c2ccccc2)c2ccccc2-4)cc1] 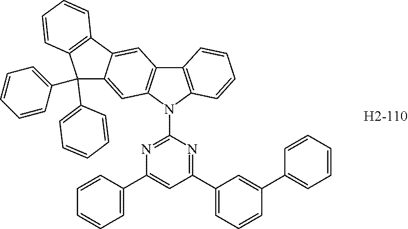 [SMILES: c1ccc(-c2cccc(-c3cc(-c4ccccc4)nc(-n4c5ccccc5c5cc6c(cc54)C(c4ccccc4)(c4ccccc4)c4ccccc4-6)n3)c2)cc1] 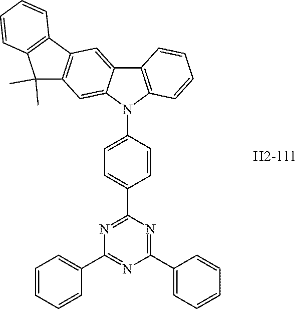 [SMILES: CC1(C)c2ccccc2-c2cc3c4ccccc4n(-c4ccc(-c5nc(-c6ccccc6)nc(-c6ccccc6)n5)cc4)c3cc21] 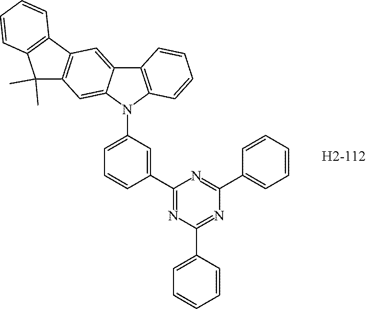 [SMILES: CC1(C)c2ccccc2-c2cc3c4ccccc4n(-c4cccc(-c5nc(-c6ccccc6)nc(-c6ccccc6)n5)c4)c3cc21] 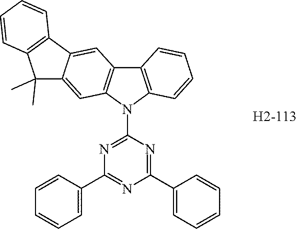 [SMILES: CC1(C)c2ccccc2-c2cc3c4ccccc4n(-c4nc(-c5ccccc5)nc(-c5ccccc5)n4)c3cc21] 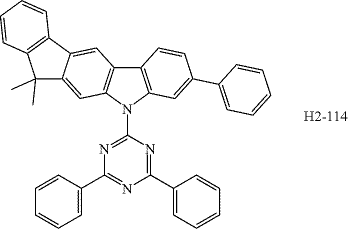 [SMILES: CC1(C)c2ccccc2-c2cc3c4ccc(-c5ccccc5)cc4n(-c4nc(-c5ccccc5)nc(-c5ccccc5)n4)c3cc21] 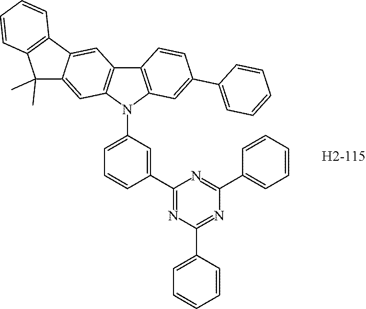 [SMILES: CC1(C)c2ccccc2-c2cc3c4ccc(-c5ccccc5)cc4n(-c4cccc(-c5nc(-c6ccccc6)nc(-c6ccccc6)n5)c4)c3cc21] 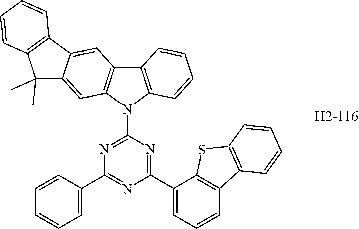 [SMILES: CC1(C)c2ccccc2-c2cc3c4ccccc4n(-c4nc(-c5ccccc5)nc(-c5cccc6c5sc5ccccc56)n4)c3cc21] 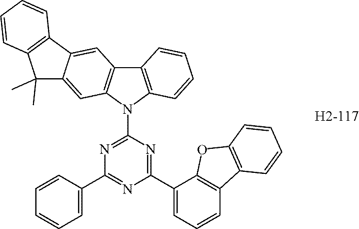 [SMILES: CC1(C)c2ccccc2-c2cc3c4ccccc4n(-c4nc(-c5ccccc5)nc(-c5cccc6c5oc5ccccc56)n4)c3cc21] 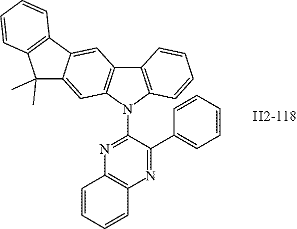 [SMILES: CC1(C)c2ccccc2-c2cc3c4ccccc4n(-c4nc5ccccc5nc4-c4ccccc4)c3cc21] 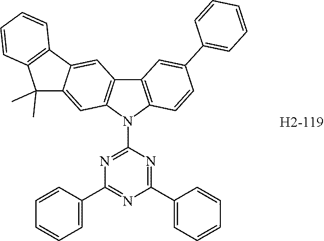 [SMILES: CC1(C)c2ccccc2-c2cc3c4cc(-c5ccccc5)ccc4n(-c4nc(-c5ccccc5)nc(-c5ccccc5)n4)c3cc21] 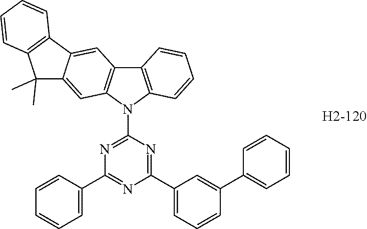 [SMILES: CC1(C)c2ccccc2-c2cc3c4ccccc4n(-c4nc(-c5ccccc5)nc(-c5cccc(-c6ccccc6)c5)n4)c3cc21] 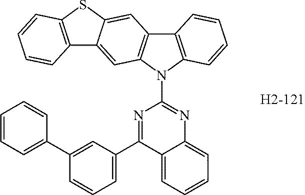 [SMILES: c1ccc(-c2cccc(-c3nc(-n4c5ccccc5c5cc6sc7ccccc7c6cc54)nc4ccccc34)c2)cc1] 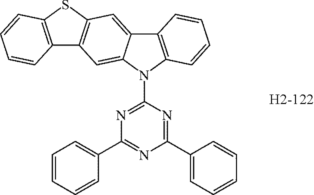 [SMILES: c1ccc(-c2nc(-c3ccccc3)nc(-n3c4ccccc4c4cc5sc6ccccc6c5cc43)n2)cc1] 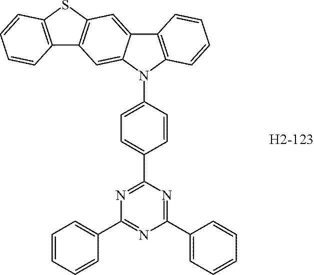 [SMILES: c1ccc(-c2nc(-c3ccccc3)nc(-c3ccc(-n4c5ccccc5c5cc6sc7ccccc7c6cc54)cc3)n2)cc1] 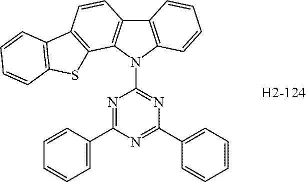 [SMILES: c1ccc(-c2nc(-c3ccccc3)nc(-n3c4ccccc4c4ccc5c6ccccc6sc5c43)n2)cc1] 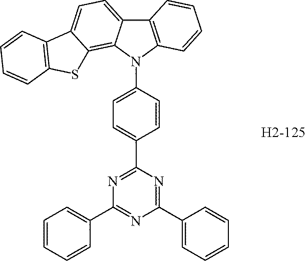 [SMILES: c1ccc(-c2nc(-c3ccccc3)nc(-c3ccc(-n4c5ccccc5c5ccc6c7ccccc7sc6c54)cc3)n2)cc1] 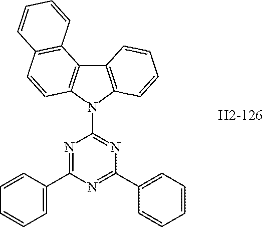 [SMILES: c1ccc(-c2nc(-c3ccccc3)nc(-n3c4ccccc4c4c5ccccc5ccc43)n2)cc1] 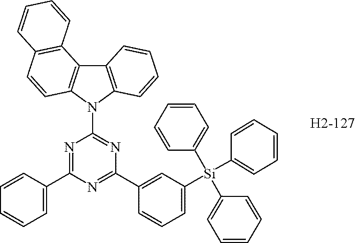 [SMILES: c1ccc(-c2nc(-c3cccc([Si](c4ccccc4)(c4ccccc4)c4ccccc4)c3)nc(-n3c4ccccc4c4c5ccccc5ccc43)n2)cc1] 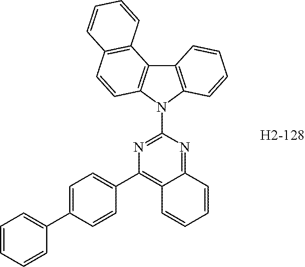 [SMILES: c1ccc(-c2ccc(-c3nc(-n4c5ccccc5c5c6ccccc6ccc54)nc4ccccc34)cc2)cc1] 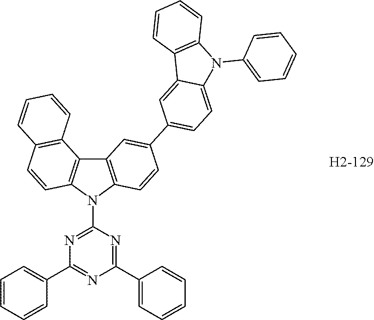 [SMILES: c1ccc(-c2nc(-c3ccccc3)nc(-n3c4ccc(-c5ccc6c(c5)c5ccccc5n6-c5ccccc5)cc4c4c5ccccc5ccc43)n2)cc1] 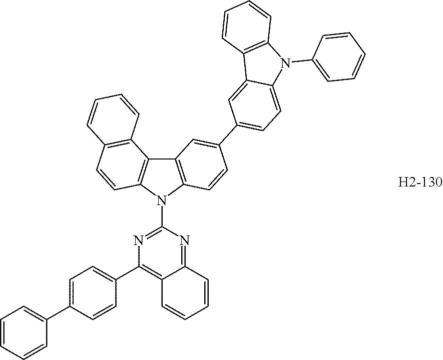 [SMILES: c1ccc(-c2ccc(-c3nc(-n4c5ccc(-c6ccc7c(c6)c6ccccc6n7-c6ccccc6)cc5c5c6ccccc6ccc54)nc4ccccc34)cc2)cc1] 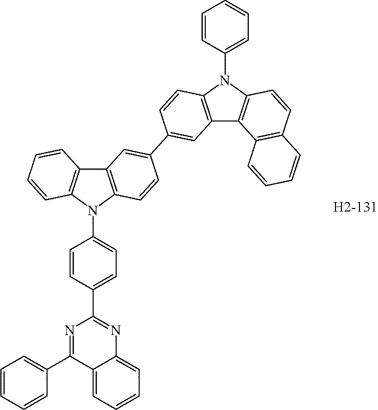 [SMILES: c1ccc(-c2nc(-c3ccc(-n4c5ccccc5c5cc(-c6ccc7c(c6)c6c8ccccc8ccc6n7-c6ccccc6)ccc54)cc3)nc3ccccc23)cc1] 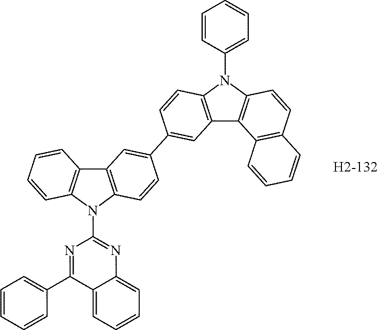 [SMILES: c1ccc(-c2nc(-n3c4ccccc4c4cc(-c5ccc6c(c5)c5c7ccccc7ccc5n6-c5ccccc5)ccc43)nc3ccccc23)cc1] 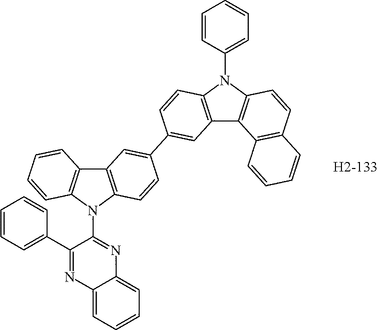 [SMILES: c1ccc(-c2nc3ccccc3nc2-n2c3ccccc3c3cc(-c4ccc5c(c4)c4c6ccccc6ccc4n5-c4ccccc4)ccc32)cc1] 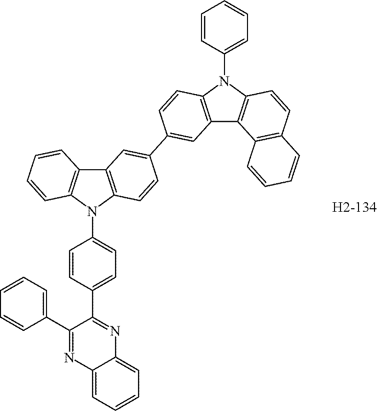 [SMILES: c1ccc(-c2nc3ccccc3nc2-c2ccc(-n3c4ccccc4c4cc(-c5ccc6c(c5)c5c7ccccc7ccc5n6-c5ccccc5)ccc43)cc2)cc1] 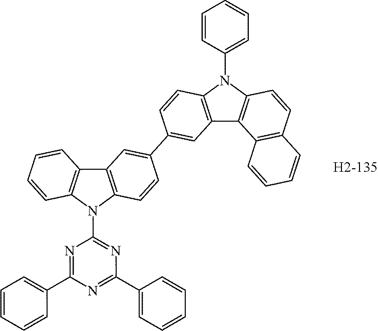 [SMILES: c1ccc(-c2nc(-c3ccccc3)nc(-n3c4ccccc4c4cc(-c5ccc6c(c5)c5c7ccccc7ccc5n6-c5ccccc5)ccc43)n2)cc1] 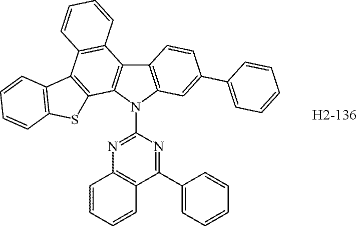 [SMILES: c1ccc(-c2ccc3c4c5ccccc5c5c6ccccc6sc5c4n(-c4nc(-c5ccccc5)c5ccccc5n4)c3c2)cc1] 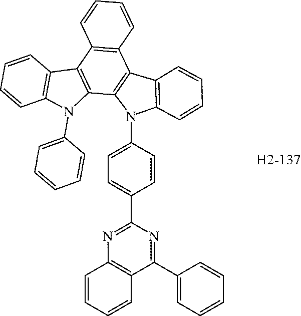 [SMILES: c1ccc(-c2nc(-c3ccc(-n4c5ccccc5c5c6ccccc6c6c7ccccc7n(-c7ccccc7)c6c54)cc3)nc3ccccc23)cc1] 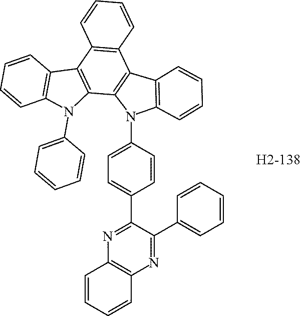 [SMILES: c1ccc(-c2nc3ccccc3nc2-c2ccc(-n3c4ccccc4c4c5ccccc5c5c6ccccc6n(-c6ccccc6)c5c43)cc2)cc1] 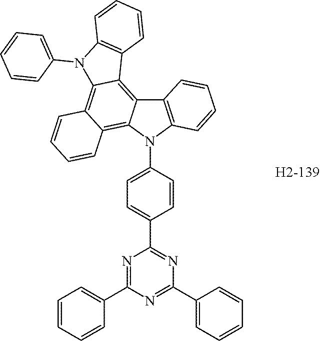 [SMILES: c1ccc(-c2nc(-c3ccccc3)nc(-c3ccc(-n4c5ccccc5c5c6c7ccccc7n(-c7ccccc7)c6c6ccccc6c54)cc3)n2)cc1] 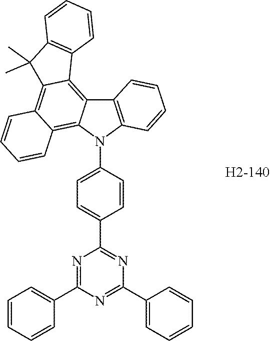 [SMILES: CC1(C)c2ccccc2-c2c1c1ccccc1c1c2c2ccccc2n1-c1ccc(-c2nc(-c3ccccc3)nc(-c3ccccc3)n2)cc1] 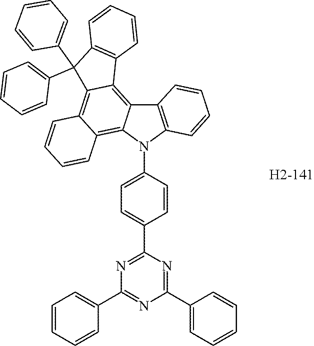 [SMILES: c1ccc(-c2nc(-c3ccccc3)nc(-c3ccc(-n4c5ccccc5c5c6c(c7ccccc7c54)C(c4ccccc4)(c4ccccc4)c4ccccc4-6)cc3)n2)cc1] 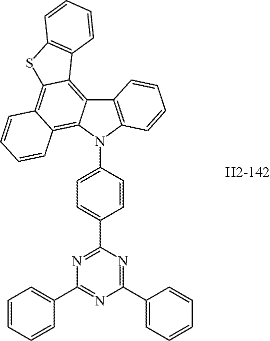 [SMILES: c1ccc(-c2nc(-c3ccccc3)nc(-c3ccc(-n4c5ccccc5c5c6c7ccccc7sc6c6ccccc6c54)cc3)n2)cc1] 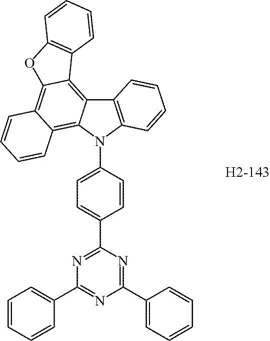 [SMILES: c1ccc(-c2nc(-c3ccccc3)nc(-c3ccc(-n4c5ccccc5c5c6c7ccccc7oc6c6ccccc6c54)cc3)n2)cc1] 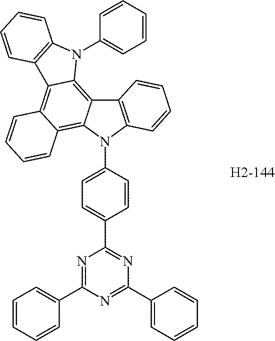 [SMILES: c1ccc(-c2nc(-c3ccccc3)nc(-c3ccc(-n4c5ccccc5c5c4c4ccccc4c4c6ccccc6n(-c6ccccc6)c45)cc3)n2)cc1] 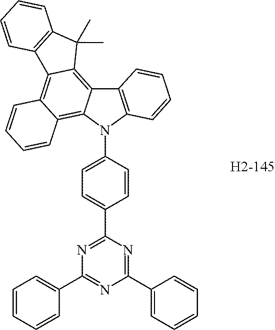 [SMILES: CC1(C)c2ccccc2-c2c1c1c3ccccc3n(-c3ccc(-c4nc(-c5ccccc5)nc(-c5ccccc5)n4)cc3)c1c1ccccc21] 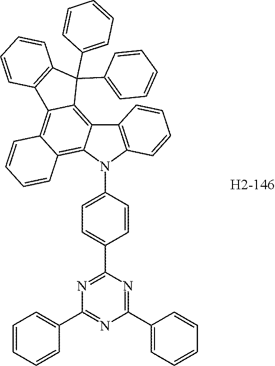 [SMILES: c1ccc(-c2nc(-c3ccccc3)nc(-c3ccc(-n4c5ccccc5c5c6c(c7ccccc7c54)-c4ccccc4C6(c4ccccc4)c4ccccc4)cc3)n2)cc1] 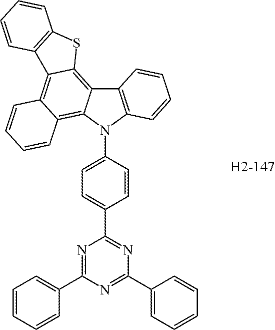 [SMILES: c1ccc(-c2nc(-c3ccccc3)nc(-c3ccc(-n4c5ccccc5c5c6sc7ccccc7c6c6ccccc6c54)cc3)n2)cc1] 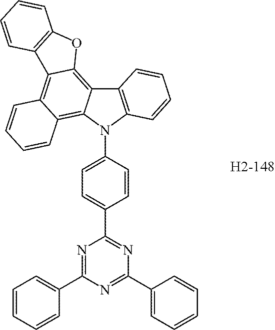 [SMILES: c1ccc(-c2nc(-c3ccccc3)nc(-c3ccc(-n4c5ccccc5c5c6oc7ccccc7c6c6ccccc6c54)cc3)n2)cc1] 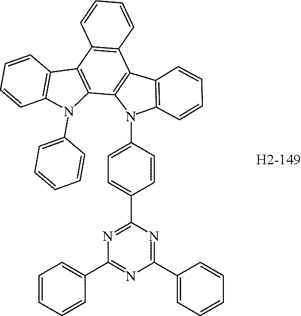 [SMILES: c1ccc(-c2nc(-c3ccccc3)nc(-c3ccc(-n4c5ccccc5c5c6ccccc6c6c7ccccc7n(-c7ccccc7)c6c54)cc3)n2)cc1] 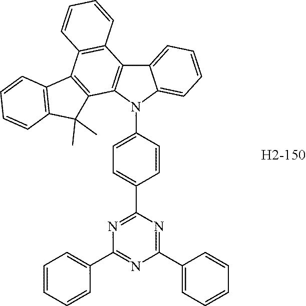 [SMILES: CC1(C)c2ccccc2-c2c1c1c(c3ccccc23)c2ccccc2n1-c1ccc(-c2nc(-c3ccccc3)nc(-c3ccccc3)n2)cc1] 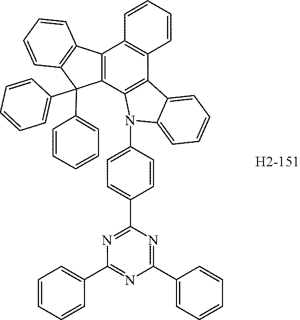 [SMILES: c1ccc(-c2nc(-c3ccccc3)nc(-c3ccc(-n4c5ccccc5c5c6ccccc6c6c(c54)C(c4ccccc4)(c4ccccc4)c4ccccc4-6)cc3)n2)cc1] 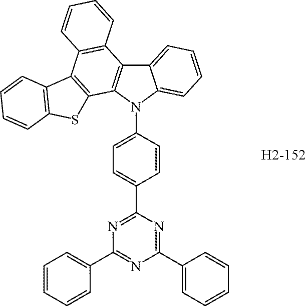 [SMILES: c1ccc(-c2nc(-c3ccccc3)nc(-c3ccc(-n4c5ccccc5c5c6ccccc6c6c7ccccc7sc6c54)cc3)n2)cc1] 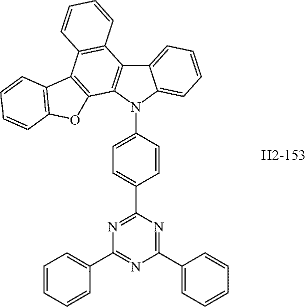 [SMILES: c1ccc(-c2nc(-c3ccccc3)nc(-c3ccc(-n4c5ccccc5c5c6ccccc6c6c7ccccc7oc6c54)cc3)n2)cc1] 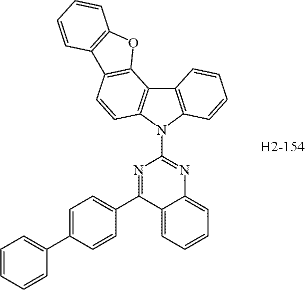 [SMILES: c1ccc(-c2ccc(-c3nc(-n4c5ccccc5c5c6oc7ccccc7c6ccc54)nc4ccccc34)cc2)cc1] 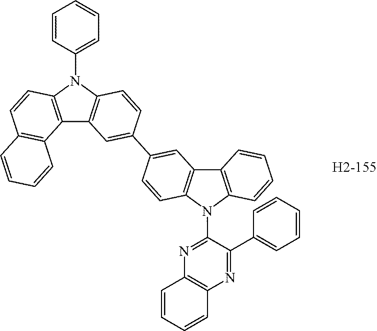 [SMILES: c1ccc(-c2nc3ccccc3nc2-n2c3ccccc3c3cc(-c4ccc5c(c4)c4c6ccccc6ccc4n5-c4ccccc4)ccc32)cc1] 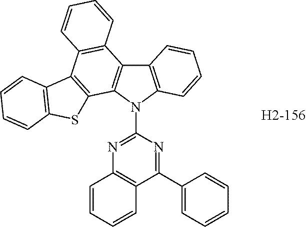 [SMILES: c1ccc(-c2nc(-n3c4ccccc4c4c5ccccc5c5c6ccccc6sc5c43)nc3ccccc23)cc1] 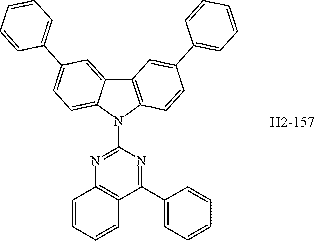 [SMILES: c1ccc(-c2ccc3c(c2)c2cc(-c4ccccc4)ccc2n3-c2nc(-c3ccccc3)c3ccccc3n2)cc1] 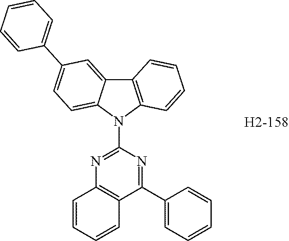 [SMILES: c1ccc(-c2ccc3c(c2)c2ccccc2n3-c2nc(-c3ccccc3)c3ccccc3n2)cc1] 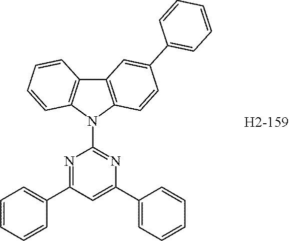 [SMILES: c1ccc(-c2ccc3c(c2)c2ccccc2n3-c2nc(-c3ccccc3)cc(-c3ccccc3)n2)cc1] 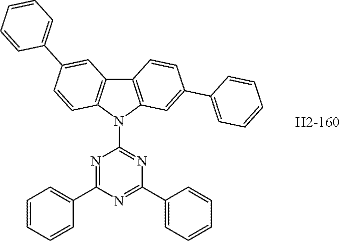 [SMILES: c1ccc(-c2ccc3c(c2)c2ccc(-c4ccccc4)cc2n3-c2nc(-c3ccccc3)nc(-c3ccccc3)n2)cc1] 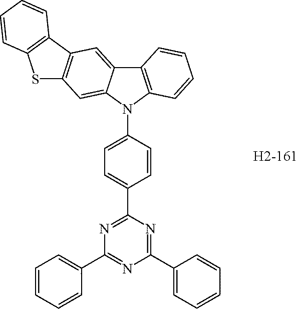 [SMILES: c1ccc(-c2nc(-c3ccccc3)nc(-c3ccc(-n4c5ccccc5c5cc6c(cc54)sc4ccccc46)cc3)n2)cc1] 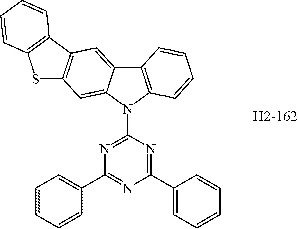 [SMILES: c1ccc(-c2nc(-c3ccccc3)nc(-n3c4ccccc4c4cc5c(cc43)sc3ccccc35)n2)cc1] 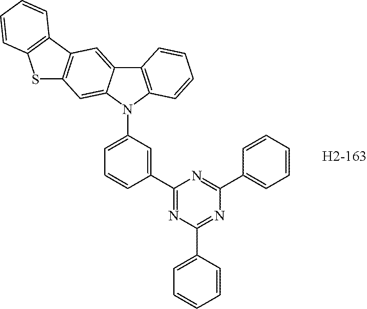 [SMILES: c1ccc(-c2nc(-c3ccccc3)nc(-c3cccc(-n4c5ccccc5c5cc6c(cc54)sc4ccccc46)c3)n2)cc1] 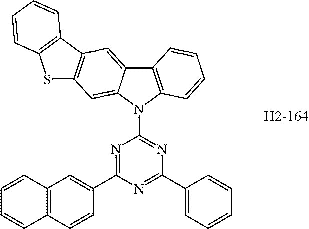 [SMILES: c1ccc(-c2nc(-c3ccc4ccccc4c3)nc(-n3c4ccccc4c4cc5c(cc43)sc3ccccc35)n2)cc1] 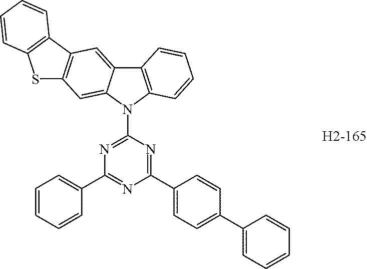 [SMILES: c1ccc(-c2ccc(-c3nc(-c4ccccc4)nc(-n4c5ccccc5c5cc6c(cc54)sc4ccccc46)n3)cc2)cc1] 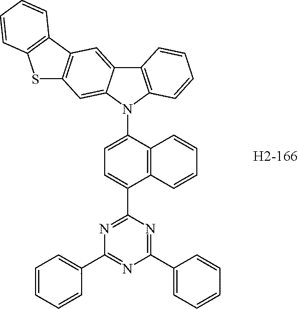 [SMILES: c1ccc(-c2nc(-c3ccccc3)nc(-c3ccc(-n4c5ccccc5c5cc6c(cc54)sc4ccccc46)c4ccccc34)n2)cc1] 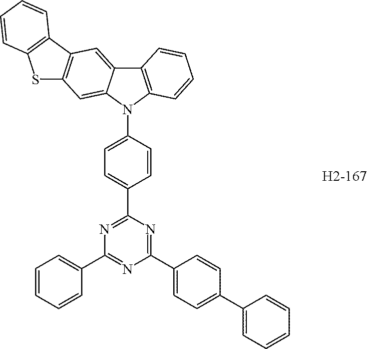 [SMILES: c1ccc(-c2ccc(-c3nc(-c4ccccc4)nc(-c4ccc(-n5c6ccccc6c6cc7c(cc65)sc5ccccc57)cc4)n3)cc2)cc1] 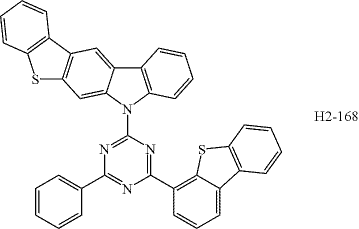 [SMILES: c1ccc(-c2nc(-c3cccc4c3sc3ccccc34)nc(-n3c4ccccc4c4cc5c(cc43)sc3ccccc35)n2)cc1] 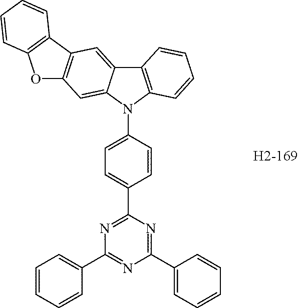 [SMILES: c1ccc(-c2nc(-c3ccccc3)nc(-c3ccc(-n4c5ccccc5c5cc6c(cc54)oc4ccccc46)cc3)n2)cc1] 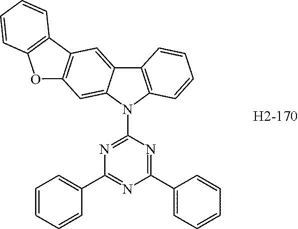 [SMILES: c1ccc(-c2nc(-c3ccccc3)nc(-n3c4ccccc4c4cc5c(cc43)oc3ccccc35)n2)cc1] 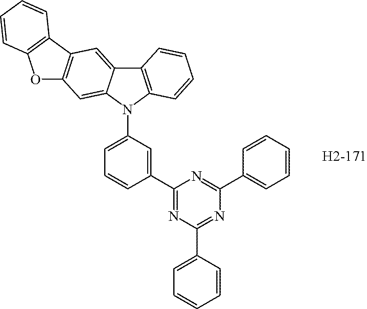 [SMILES: c1ccc(-c2nc(-c3ccccc3)nc(-c3cccc(-n4c5ccccc5c5cc6c(cc54)oc4ccccc46)c3)n2)cc1] 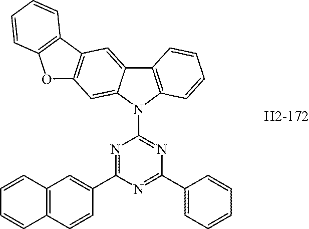 [SMILES: c1ccc(-c2nc(-c3ccc4ccccc4c3)nc(-n3c4ccccc4c4cc5c(cc43)oc3ccccc35)n2)cc1] 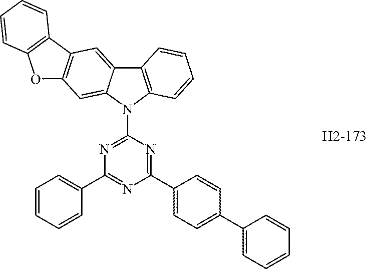 [SMILES: c1ccc(-c2ccc(-c3nc(-c4ccccc4)nc(-n4c5ccccc5c5cc6c(cc54)oc4ccccc46)n3)cc2)cc1] 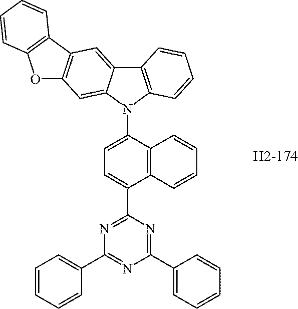 [SMILES: c1ccc(-c2nc(-c3ccccc3)nc(-c3ccc(-n4c5ccccc5c5cc6c(cc54)oc4ccccc46)c4ccccc34)n2)cc1] 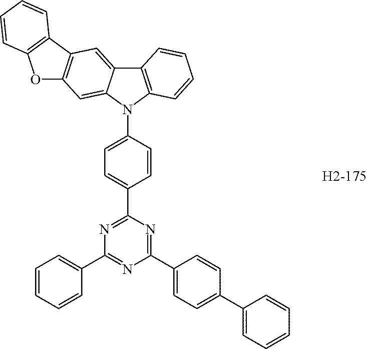 [SMILES: c1ccc(-c2ccc(-c3nc(-c4ccccc4)nc(-c4ccc(-n5c6ccccc6c6cc7c(cc65)oc5ccccc57)cc4)n3)cc2)cc1] 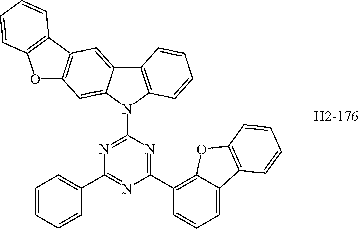 [SMILES: c1ccc(-c2nc(-c3cccc4c3oc3ccccc34)nc(-n3c4ccccc4c4cc5c(cc43)oc3ccccc35)n2)cc1] 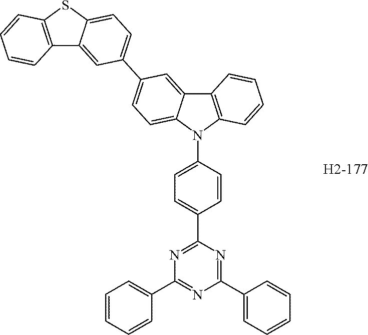 [SMILES: c1ccc(-c2nc(-c3ccccc3)nc(-c3ccc(-n4c5ccccc5c5cc(-c6ccc7sc8ccccc8c7c6)ccc54)cc3)n2)cc1] 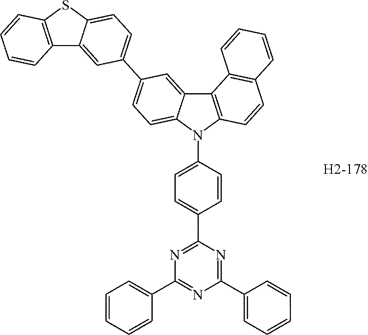 [SMILES: c1ccc(-c2nc(-c3ccccc3)nc(-c3ccc(-n4c5ccc(-c6ccc7sc8ccccc8c7c6)cc5c5c6ccccc6ccc54)cc3)n2)cc1] 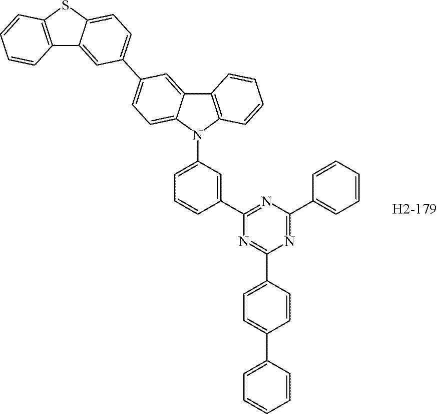 [SMILES: c1ccc(-c2ccc(-c3nc(-c4ccccc4)nc(-c4cccc(-n5c6ccccc6c6cc(-c7ccc8sc9ccccc9c8c7)ccc65)c4)n3)cc2)cc1] 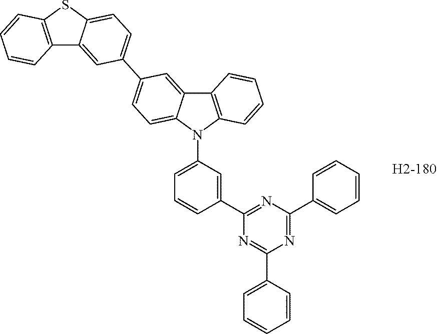 [SMILES: c1ccc(-c2nc(-c3ccccc3)nc(-c3cccc(-n4c5ccccc5c5cc(-c6ccc7sc8ccccc8c7c6)ccc54)c3)n2)cc1] 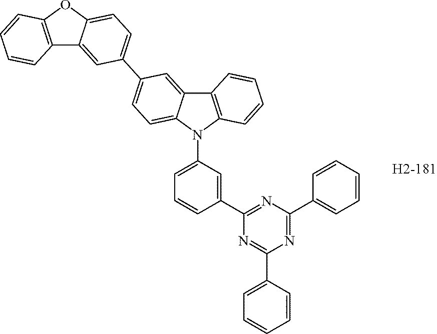 [SMILES: c1ccc(-c2nc(-c3ccccc3)nc(-c3cccc(-n4c5ccccc5c5cc(-c6ccc7oc8ccccc8c7c6)ccc54)c3)n2)cc1] 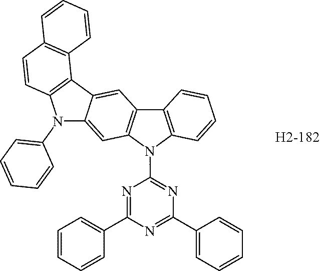 [SMILES: c1ccc(-c2nc(-c3ccccc3)nc(-n3c4ccccc4c4cc5c6c7ccccc7ccc6n(-c6ccccc6)c5cc43)n2)cc1] 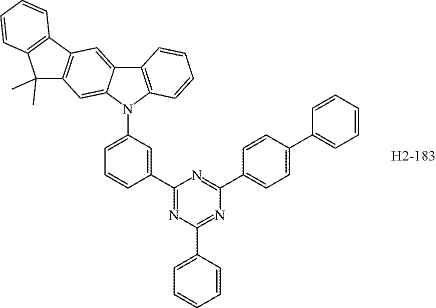 [SMILES: CC1(C)c2ccccc2-c2cc3c4ccccc4n(-c4cccc(-c5nc(-c6ccccc6)nc(-c6ccc(-c7ccccc7)cc6)n5)c4)c3cc21] 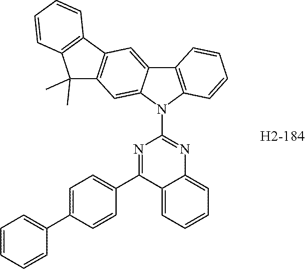 [SMILES: CC1(C)c2ccccc2-c2cc3c4ccccc4n(-c4nc(-c5ccc(-c6ccccc6)cc5)c5ccccc5n4)c3cc21] 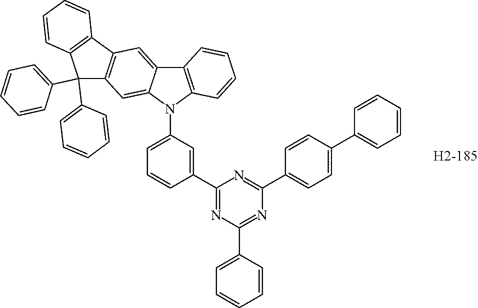 [SMILES: c1ccc(-c2ccc(-c3nc(-c4ccccc4)nc(-c4cccc(-n5c6ccccc6c6cc7c(cc65)C(c5ccccc5)(c5ccccc5)c5ccccc5-7)c4)n3)cc2)cc1] 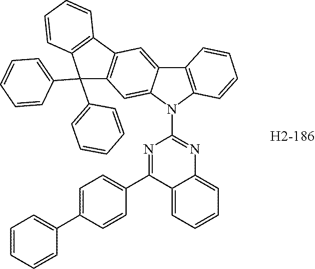 [SMILES: c1ccc(-c2ccc(-c3nc(-n4c5ccccc5c5cc6c(cc54)C(c4ccccc4)(c4ccccc4)c4ccccc4-6)nc4ccccc34)cc2)cc1] 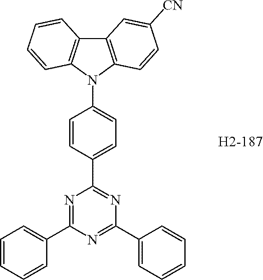 [SMILES: N#Cc1ccc2c(c1)c1ccccc1n2-c1ccc(-c2nc(-c3ccccc3)nc(-c3ccccc3)n2)cc1] 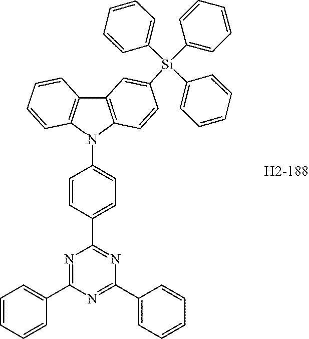 [SMILES: c1ccc(-c2nc(-c3ccccc3)nc(-c3ccc(-n4c5ccccc5c5cc([Si](c6ccccc6)(c6ccccc6)c6ccccc6)ccc54)cc3)n2)cc1] 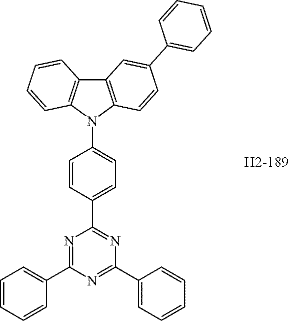 [SMILES: c1ccc(-c2ccc3c(c2)c2ccccc2n3-c2ccc(-c3nc(-c4ccccc4)nc(-c4ccccc4)n3)cc2)cc1] 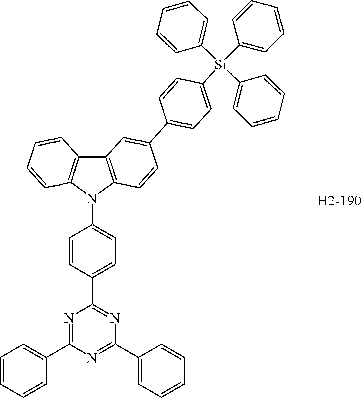 [SMILES: c1ccc(-c2nc(-c3ccccc3)nc(-c3ccc(-n4c5ccccc5c5cc(-c6ccc([Si](c7ccccc7)(c7ccccc7)c7ccccc7)cc6)ccc54)cc3)n2)cc1] 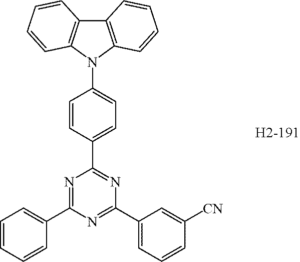 [SMILES: N#Cc1cccc(-c2nc(-c3ccccc3)nc(-c3ccc(-n4c5ccccc5c5ccccc54)cc3)n2)c1] 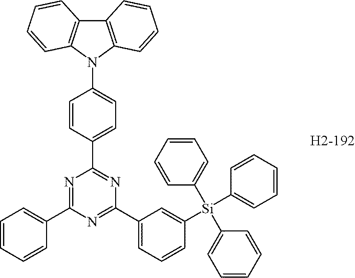 [SMILES: c1ccc(-c2nc(-c3ccc(-n4c5ccccc5c5ccccc54)cc3)nc(-c3cccc([Si](c4ccccc4)(c4ccccc4)c4ccccc4)c3)n2)cc1] 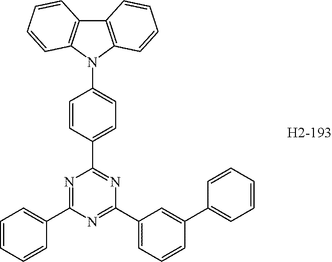 [SMILES: c1ccc(-c2cccc(-c3nc(-c4ccccc4)nc(-c4ccc(-n5c6ccccc6c6ccccc65)cc4)n3)c2)cc1] 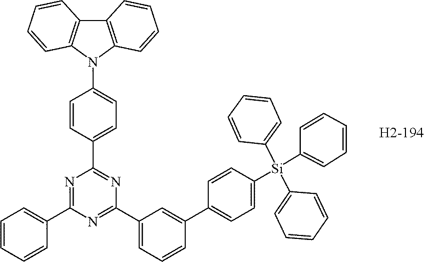 [SMILES: c1ccc(-c2nc(-c3ccc(-n4c5ccccc5c5ccccc54)cc3)nc(-c3cccc(-c4ccc([Si](c5ccccc5)(c5ccccc5)c5ccccc5)cc4)c3)n2)cc1] 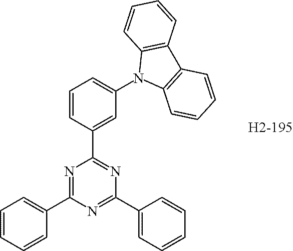 [SMILES: c1ccc(-c2nc(-c3ccccc3)nc(-c3cccc(-n4c5ccccc5c5ccccc54)c3)n2)cc1] 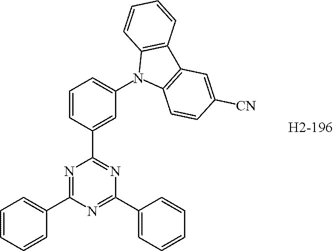 [SMILES: N#Cc1ccc2c(c1)c1ccccc1n2-c1cccc(-c2nc(-c3ccccc3)nc(-c3ccccc3)n2)c1] 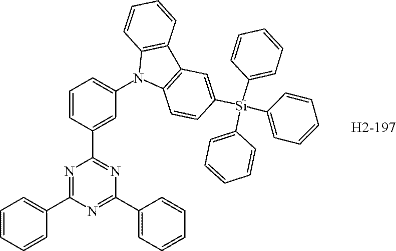 [SMILES: c1ccc(-c2nc(-c3ccccc3)nc(-c3cccc(-n4c5ccccc5c5cc([Si](c6ccccc6)(c6ccccc6)c6ccccc6)ccc54)c3)n2)cc1] 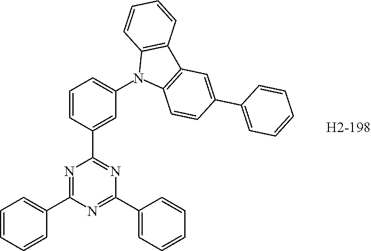 [SMILES: c1ccc(-c2ccc3c(c2)c2ccccc2n3-c2cccc(-c3nc(-c4ccccc4)nc(-c4ccccc4)n3)c2)cc1] 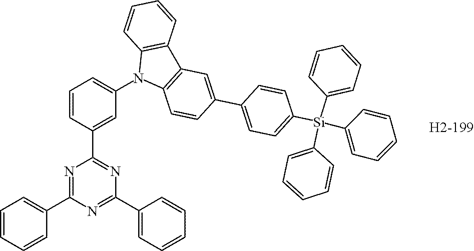 [SMILES: c1ccc(-c2nc(-c3ccccc3)nc(-c3cccc(-n4c5ccccc5c5cc(-c6ccc([Si](c7ccccc7)(c7ccccc7)c7ccccc7)cc6)ccc54)c3)n2)cc1] 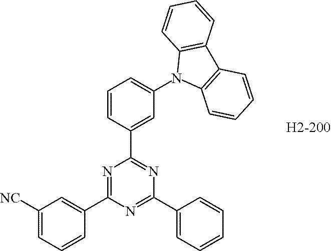 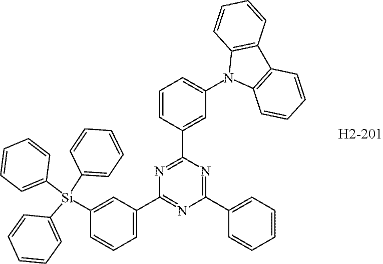 [SMILES: c1ccc(-c2nc(-c3cccc(-n4c5ccccc5c5ccccc54)c3)nc(-c3cccc([Si](c4ccccc4)(c4ccccc4)c4ccccc4)c3)n2)cc1] 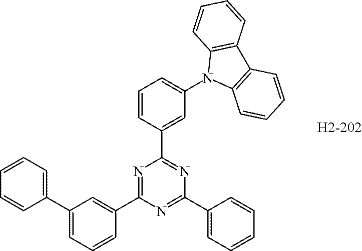 [SMILES: c1ccc(-c2cccc(-c3nc(-c4ccccc4)nc(-c4cccc(-n5c6ccccc6c6ccccc65)c4)n3)c2)cc1] 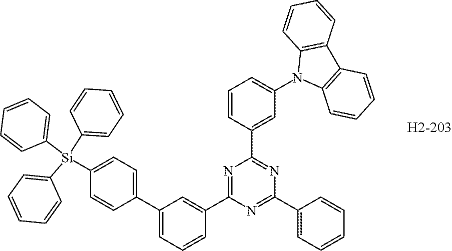 [SMILES: c1ccc(-c2nc(-c3cccc(-c4ccc([Si](c5ccccc5)(c5ccccc5)c5ccccc5)cc4)c3)nc(-c3cccc(-n4c5ccccc5c5ccccc54)c3)n2)cc1] 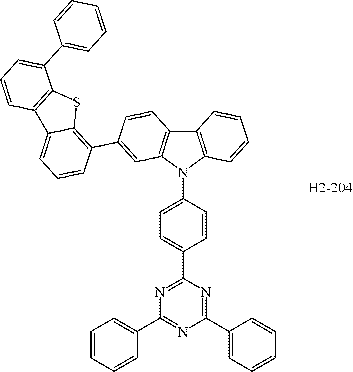 [SMILES: c1ccc(-c2nc(-c3ccccc3)nc(-c3ccc(-n4c5ccccc5c5ccc(-c6cccc7c6sc6c(-c8ccccc8)cccc67)cc54)cc3)n2)cc1] 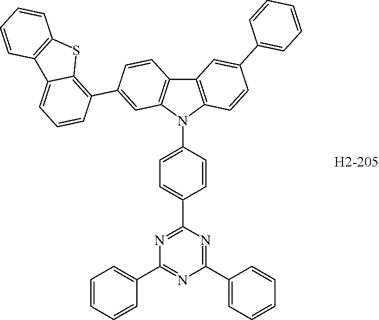 [SMILES: c1ccc(-c2ccc3c(c2)c2ccc(-c4cccc5c4sc4ccccc45)cc2n3-c2ccc(-c3nc(-c4ccccc4)nc(-c4ccccc4)n3)cc2)cc1] 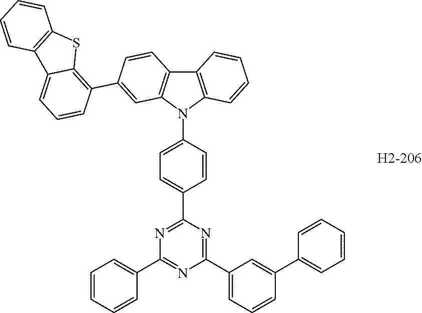 [SMILES: c1ccc(-c2cccc(-c3nc(-c4ccccc4)nc(-c4ccc(-n5c6ccccc6c6ccc(-c7cccc8c7sc7ccccc78)cc65)cc4)n3)c2)cc1] 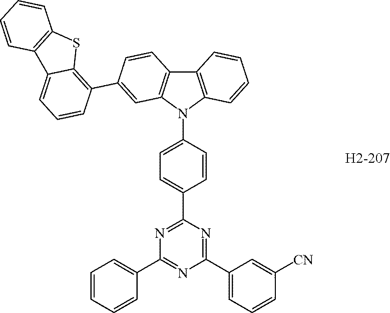 [SMILES: N#Cc1cccc(-c2nc(-c3ccccc3)nc(-c3ccc(-n4c5ccccc5c5ccc(-c6cccc7c6sc6ccccc67)cc54)cc3)n2)c1] 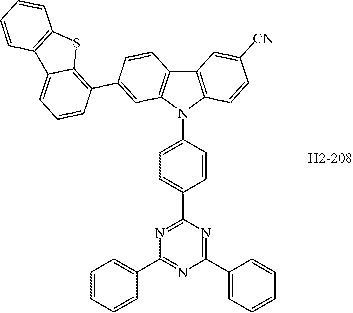 [SMILES: N#Cc1ccc2c(c1)c1ccc(-c3cccc4c3sc3ccccc34)cc1n2-c1ccc(-c2nc(-c3ccccc3)nc(-c3ccccc3)n2)cc1] 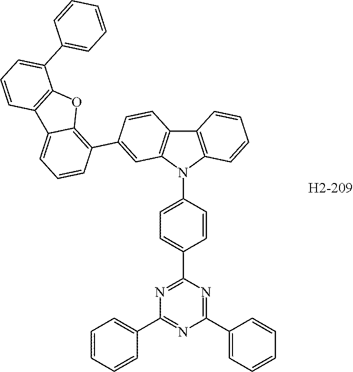 [SMILES: c1ccc(-c2nc(-c3ccccc3)nc(-c3ccc(-n4c5ccccc5c5ccc(-c6cccc7c6oc6c(-c8ccccc8)cccc67)cc54)cc3)n2)cc1] 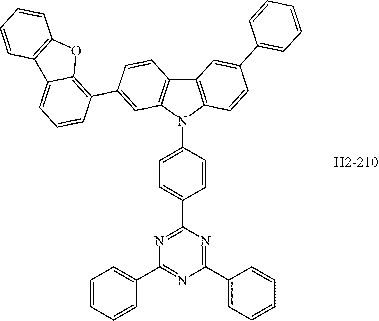 [SMILES: c1ccc(-c2ccc3c(c2)c2ccc(-c4cccc5c4oc4ccccc45)cc2n3-c2ccc(-c3nc(-c4ccccc4)nc(-c4ccccc4)n3)cc2)cc1] 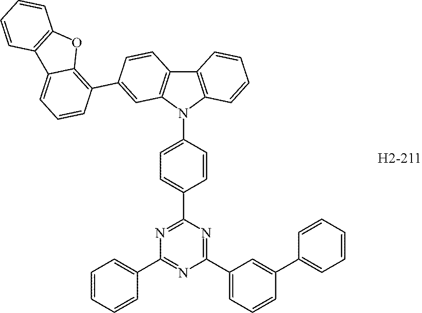 [SMILES: c1ccc(-c2cccc(-c3nc(-c4ccccc4)nc(-c4ccc(-n5c6ccccc6c6ccc(-c7cccc8c7oc7ccccc78)cc65)cc4)n3)c2)cc1] 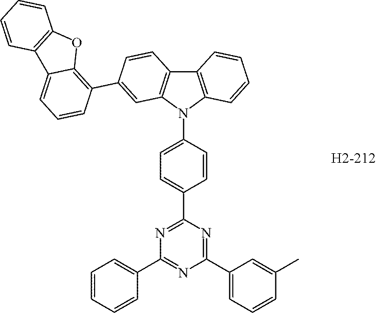 [SMILES: Cc1cccc(-c2nc(-c3ccccc3)nc(-c3ccc(-n4c5ccccc5c5ccc(-c6cccc7c6oc6ccccc67)cc54)cc3)n2)c1] 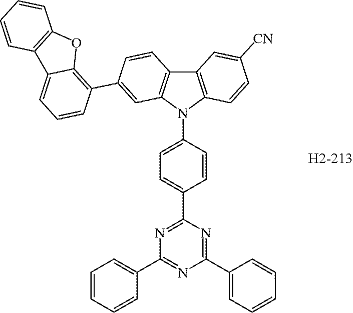 [SMILES: N#Cc1ccc2c(c1)c1ccc(-c3cccc4c3oc3ccccc34)cc1n2-c1ccc(-c2nc(-c3ccccc3)nc(-c3ccccc3)n2)cc1] 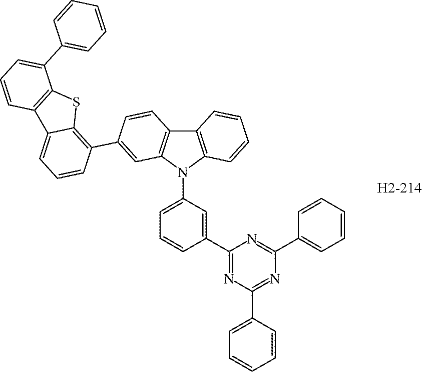 [SMILES: c1ccc(-c2nc(-c3ccccc3)nc(-c3cccc(-n4c5ccccc5c5ccc(-c6cccc7c6sc6c(-c8ccccc8)cccc67)cc54)c3)n2)cc1] 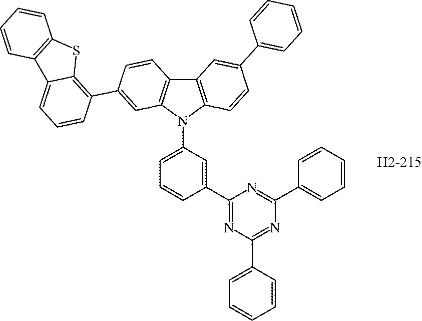 [SMILES: c1ccc(-c2ccc3c(c2)c2ccc(-c4cccc5c4sc4ccccc45)cc2n3-c2cccc(-c3nc(-c4ccccc4)nc(-c4ccccc4)n3)c2)cc1] 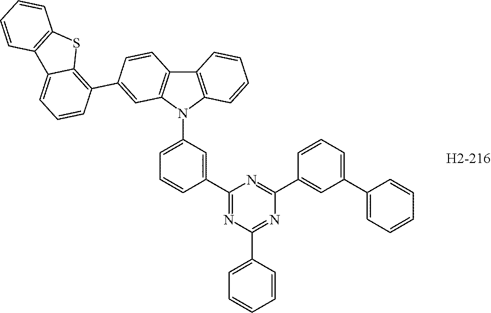 [SMILES: c1ccc(-c2cccc(-c3nc(-c4ccccc4)nc(-c4cccc(-n5c6ccccc6c6ccc(-c7cccc8c7sc7ccccc78)cc65)c4)n3)c2)cc1] 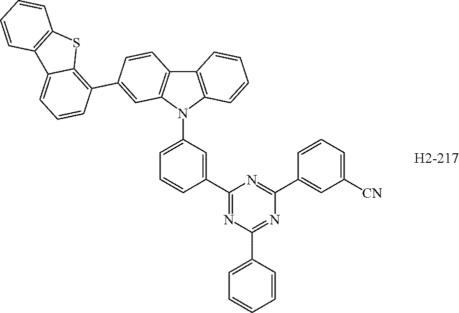 [SMILES: N#Cc1cccc(-c2nc(-c3ccccc3)nc(-c3cccc(-n4c5ccccc5c5ccc(-c6cccc7c6sc6ccccc67)cc54)c3)n2)c1] 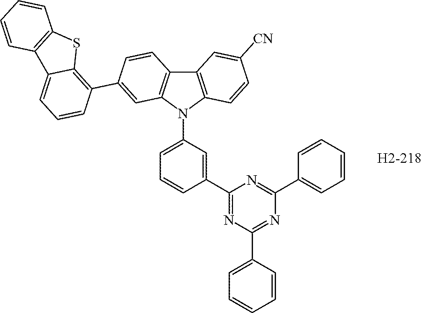 [SMILES: N#Cc1ccc2c(c1)c1ccc(-c3cccc4c3sc3ccccc34)cc1n2-c1cccc(-c2nc(-c3ccccc3)nc(-c3ccccc3)n2)c1] 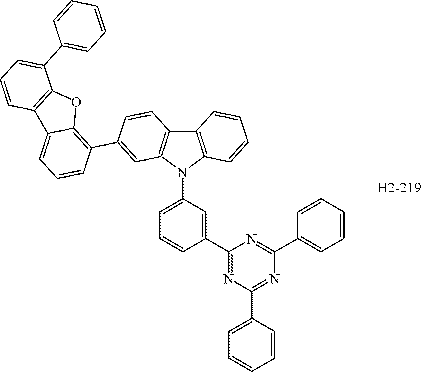 [SMILES: c1ccc(-c2nc(-c3ccccc3)nc(-c3cccc(-n4c5ccccc5c5ccc(-c6cccc7c6oc6c(-c8ccccc8)cccc67)cc54)c3)n2)cc1] 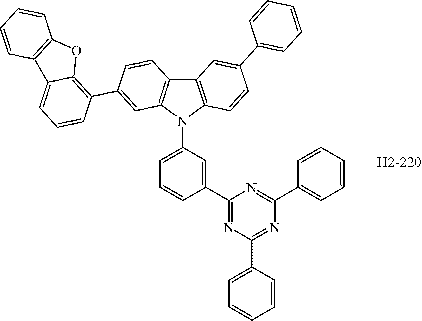 [SMILES: c1ccc(-c2ccc3c(c2)c2ccc(-c4cccc5c4oc4ccccc45)cc2n3-c2cccc(-c3nc(-c4ccccc4)nc(-c4ccccc4)n3)c2)cc1] 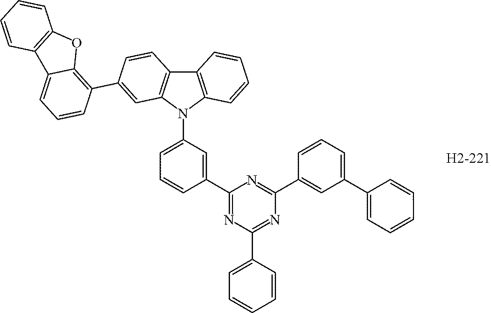 [SMILES: c1ccc(-c2cccc(-c3nc(-c4ccccc4)nc(-c4cccc(-n5c6ccccc6c6ccc(-c7cccc8c7oc7ccccc78)cc65)c4)n3)c2)cc1] 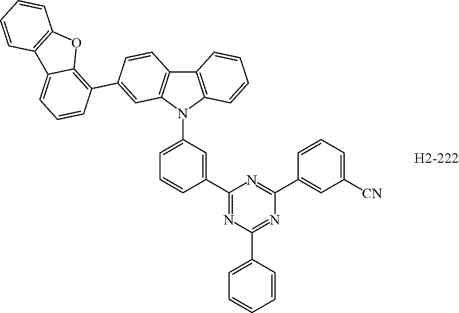 [SMILES: N#Cc1cccc(-c2nc(-c3ccccc3)nc(-c3cccc(-n4c5ccccc5c5ccc(-c6cccc7c6oc6ccccc67)cc54)c3)n2)c1] 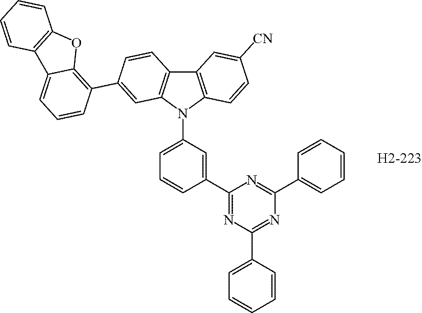 [SMILES: N#Cc1ccc2c(c1)c1ccc(-c3cccc4c3oc3ccccc34)cc1n2-c1cccc(-c2nc(-c3ccccc3)nc(-c3ccccc3)n2)c1] 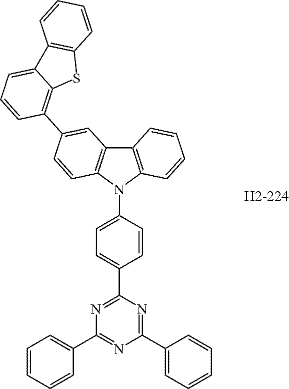 [SMILES: c1ccc(-c2nc(-c3ccccc3)nc(-c3ccc(-n4c5ccccc5c5cc(-c6cccc7c6sc6ccccc67)ccc54)cc3)n2)cc1] 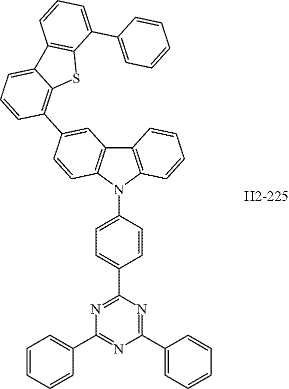 [SMILES: c1ccc(-c2nc(-c3ccccc3)nc(-c3ccc(-n4c5ccccc5c5cc(-c6cccc7c6sc6c(-c8ccccc8)cccc67)ccc54)cc3)n2)cc1] 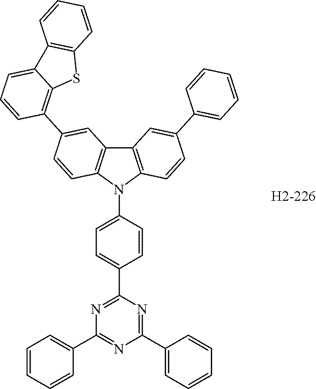 [SMILES: c1ccc(-c2ccc3c(c2)c2cc(-c4cccc5c4sc4ccccc45)ccc2n3-c2ccc(-c3nc(-c4ccccc4)nc(-c4ccccc4)n3)cc2)cc1] 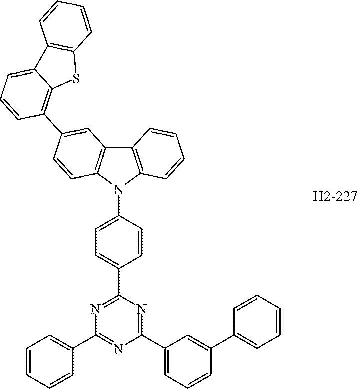 [SMILES: c1ccc(-c2cccc(-c3nc(-c4ccccc4)nc(-c4ccc(-n5c6ccccc6c6cc(-c7cccc8c7sc7ccccc78)ccc65)cc4)n3)c2)cc1] 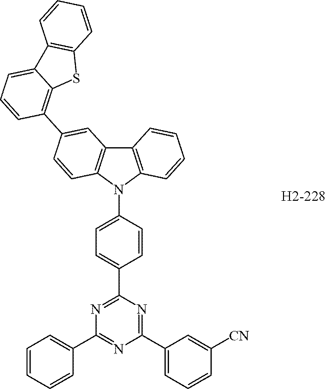 [SMILES: N#Cc1cccc(-c2nc(-c3ccccc3)nc(-c3ccc(-n4c5ccccc5c5cc(-c6cccc7c6sc6ccccc67)ccc54)cc3)n2)c1] 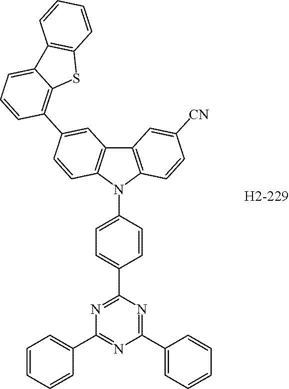 [SMILES: N#Cc1ccc2c(c1)c1cc(-c3cccc4c3sc3ccccc34)ccc1n2-c1ccc(-c2nc(-c3ccccc3)nc(-c3ccccc3)n2)cc1] 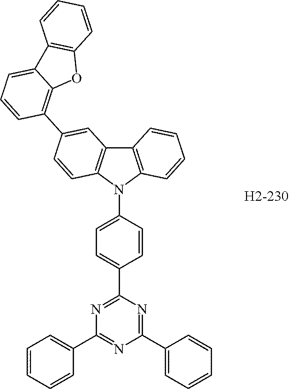 [SMILES: c1ccc(-c2nc(-c3ccccc3)nc(-c3ccc(-n4c5ccccc5c5cc(-c6cccc7c6oc6ccccc67)ccc54)cc3)n2)cc1] 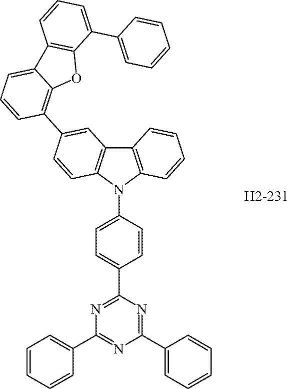 [SMILES: c1ccc(-c2nc(-c3ccccc3)nc(-c3ccc(-n4c5ccccc5c5cc(-c6cccc7c6oc6c(-c8ccccc8)cccc67)ccc54)cc3)n2)cc1] 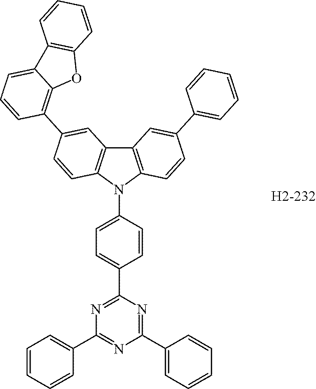 [SMILES: c1ccc(-c2ccc3c(c2)c2cc(-c4cccc5c4oc4ccccc45)ccc2n3-c2ccc(-c3nc(-c4ccccc4)nc(-c4ccccc4)n3)cc2)cc1] 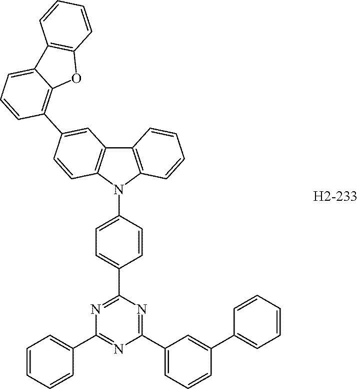 [SMILES: c1ccc(-c2cccc(-c3nc(-c4ccccc4)nc(-c4ccc(-n5c6ccccc6c6cc(-c7cccc8c7oc7ccccc78)ccc65)cc4)n3)c2)cc1] 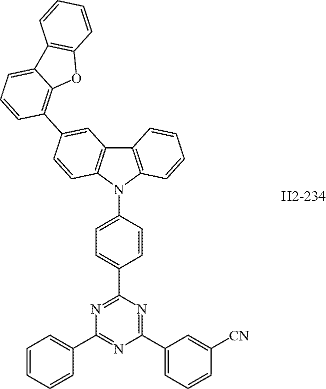 [SMILES: N#Cc1cccc(-c2nc(-c3ccccc3)nc(-c3ccc(-n4c5ccccc5c5cc(-c6cccc7c6oc6ccccc67)ccc54)cc3)n2)c1] 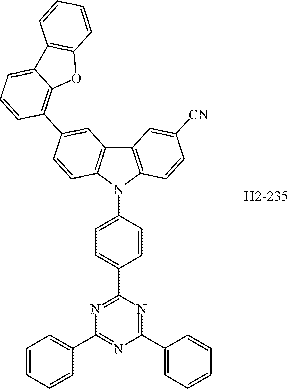 [SMILES: N#Cc1ccc2c(c1)c1cc(-c3cccc4c3oc3ccccc34)ccc1n2-c1ccc(-c2nc(-c3ccccc3)nc(-c3ccccc3)n2)cc1] 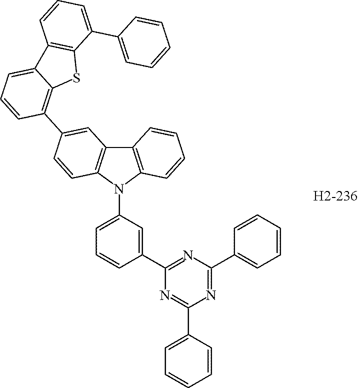 [SMILES: c1ccc(-c2nc(-c3ccccc3)nc(-c3cccc(-n4c5ccccc5c5cc(-c6cccc7c6sc6c(-c8ccccc8)cccc67)ccc54)c3)n2)cc1] 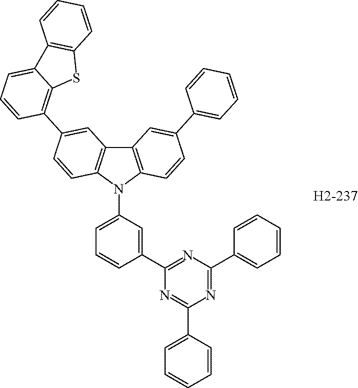 [SMILES: c1ccc(-c2ccc3c(c2)c2cc(-c4cccc5c4sc4ccccc45)ccc2n3-c2cccc(-c3nc(-c4ccccc4)nc(-c4ccccc4)n3)c2)cc1] 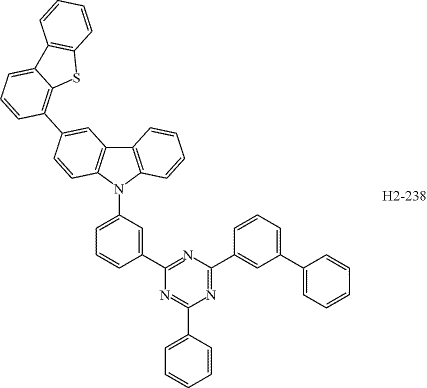 [SMILES: c1ccc(-c2cccc(-c3nc(-c4ccccc4)nc(-c4cccc(-n5c6ccccc6c6cc(-c7cccc8c7sc7ccccc78)ccc65)c4)n3)c2)cc1] 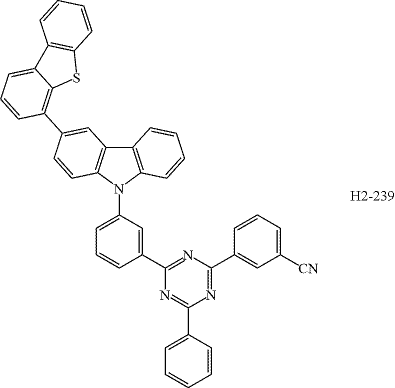 [SMILES: N#Cc1cccc(-c2nc(-c3ccccc3)nc(-c3cccc(-n4c5ccccc5c5cc(-c6cccc7c6sc6ccccc67)ccc54)c3)n2)c1] 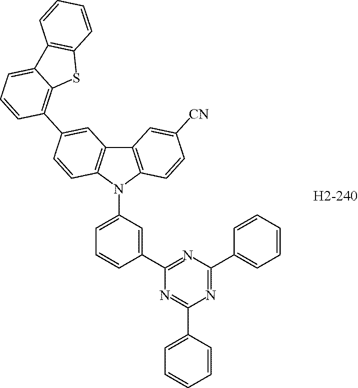 [SMILES: N#Cc1ccc2c(c1)c1cc(-c3cccc4c3sc3ccccc34)ccc1n2-c1cccc(-c2nc(-c3ccccc3)nc(-c3ccccc3)n2)c1] 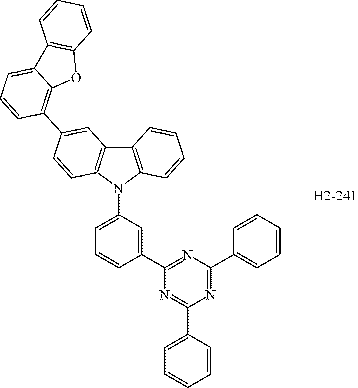 [SMILES: c1ccc(-c2nc(-c3ccccc3)nc(-c3cccc(-n4c5ccccc5c5cc(-c6cccc7c6oc6ccccc67)ccc54)c3)n2)cc1] 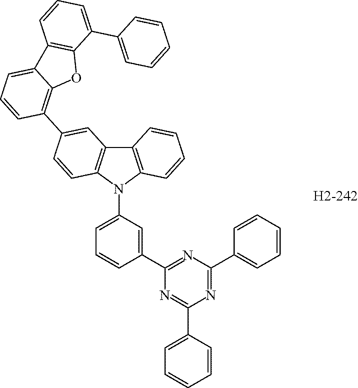 [SMILES: c1ccc(-c2nc(-c3ccccc3)nc(-c3cccc(-n4c5ccccc5c5cc(-c6cccc7c6oc6c(-c8ccccc8)cccc67)ccc54)c3)n2)cc1] 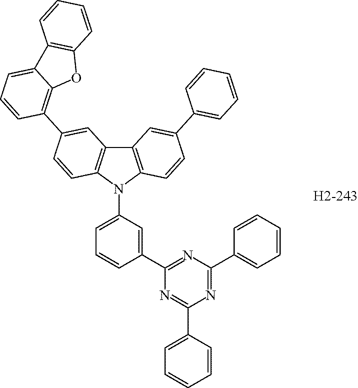 [SMILES: c1ccc(-c2ccc3c(c2)c2cc(-c4cccc5c4oc4ccccc45)ccc2n3-c2cccc(-c3nc(-c4ccccc4)nc(-c4ccccc4)n3)c2)cc1] 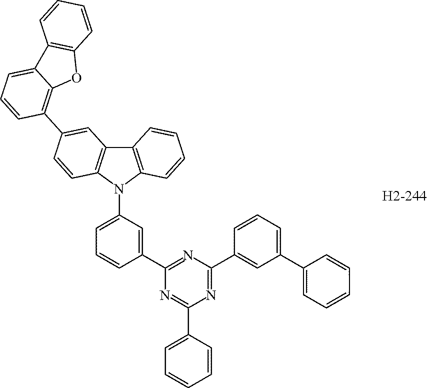 [SMILES: c1ccc(-c2cccc(-c3nc(-c4ccccc4)nc(-c4cccc(-n5c6ccccc6c6cc(-c7cccc8c7oc7ccccc78)ccc65)c4)n3)c2)cc1] 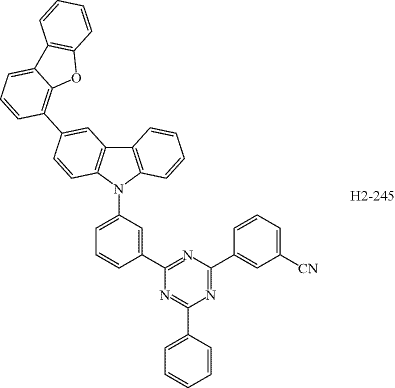 [SMILES: N#Cc1cccc(-c2nc(-c3ccccc3)nc(-c3cccc(-n4c5ccccc5c5cc(-c6cccc7c6oc6ccccc67)ccc54)c3)n2)c1] 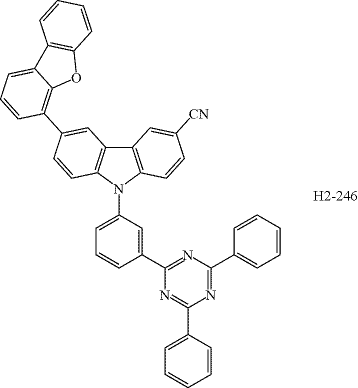 [SMILES: N#Cc1ccc2c(c1)c1cc(-c3cccc4c3oc3ccccc34)ccc1n2-c1cccc(-c2nc(-c3ccccc3)nc(-c3ccccc3)n2)c1] 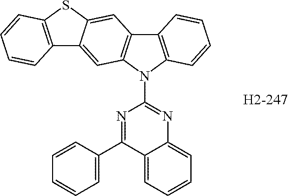 [SMILES: c1ccc(-c2nc(-n3c4ccccc4c4cc5sc6ccccc6c5cc43)nc3ccccc23)cc1] 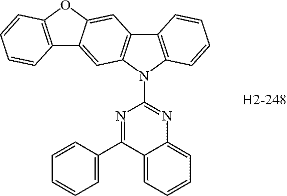 [SMILES: c1ccc(-c2nc(-n3c4ccccc4c4cc5oc6ccccc6c5cc43)nc3ccccc23)cc1] 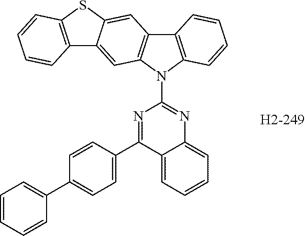 [SMILES: c1ccc(-c2ccc(-c3nc(-n4c5ccccc5c5cc6sc7ccccc7c6cc54)nc4ccccc34)cc2)cc1] 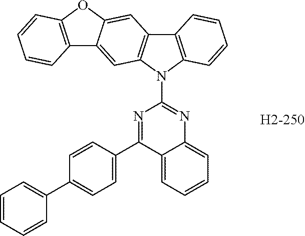 [SMILES: c1ccc(-c2ccc(-c3nc(-n4c5ccccc5c5cc6oc7ccccc7c6cc54)nc4ccccc34)cc2)cc1] 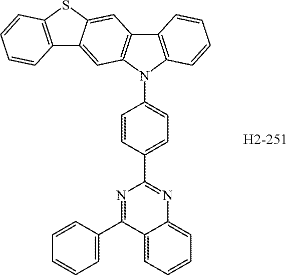 [SMILES: c1ccc(-c2nc(-c3ccc(-n4c5ccccc5c5cc6sc7ccccc7c6cc54)cc3)nc3ccccc23)cc1] 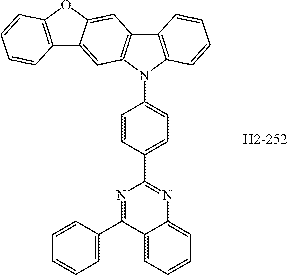 [SMILES: c1ccc(-c2nc(-c3ccc(-n4c5ccccc5c5cc6oc7ccccc7c6cc54)cc3)nc3ccccc23)cc1] 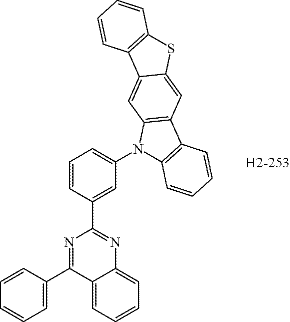 [SMILES: c1ccc(-c2nc(-c3cccc(-n4c5ccccc5c5cc6sc7ccccc7c6cc54)c3)nc3ccccc23)cc1] 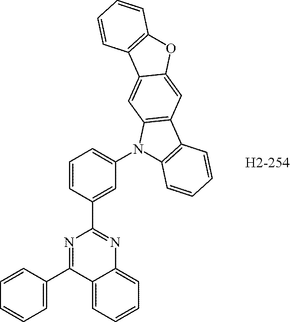 [SMILES: c1ccc(-c2nc(-c3cccc(-n4c5ccccc5c5cc6oc7ccccc7c6cc54)c3)nc3ccccc23)cc1] 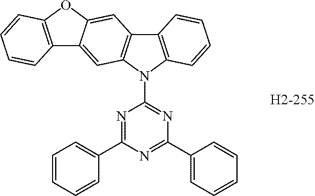 [SMILES: c1ccc(-c2nc(-c3ccccc3)nc(-n3c4ccccc4c4cc5oc6ccccc6c5cc43)n2)cc1] 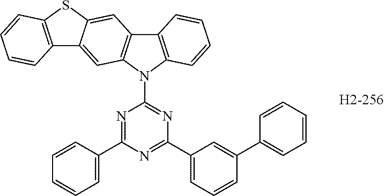 [SMILES: c1ccc(-c2cccc(-c3nc(-c4ccccc4)nc(-n4c5ccccc5c5cc6sc7ccccc7c6cc54)n3)c2)cc1] 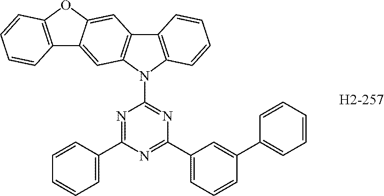 [SMILES: c1ccc(-c2cccc(-c3nc(-c4ccccc4)nc(-n4c5ccccc5c5cc6oc7ccccc7c6cc54)n3)c2)cc1] 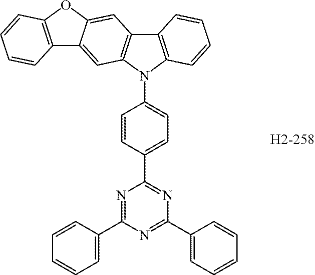 [SMILES: c1ccc(-c2nc(-c3ccccc3)nc(-c3ccc(-n4c5ccccc5c5cc6oc7ccccc7c6cc54)cc3)n2)cc1] 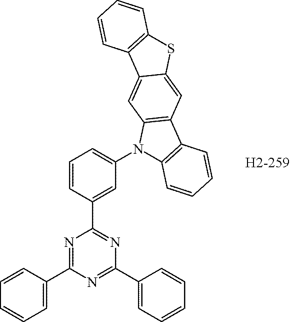 [SMILES: c1ccc(-c2nc(-c3ccccc3)nc(-c3cccc(-n4c5ccccc5c5cc6sc7ccccc7c6cc54)c3)n2)cc1] 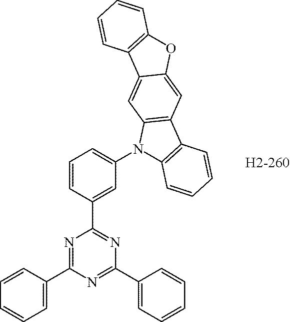 [SMILES: c1ccc(-c2nc(-c3ccccc3)nc(-c3cccc(-n4c5ccccc5c5cc6oc7ccccc7c6cc54)c3)n2)cc1] 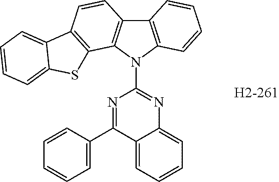 [SMILES: c1ccc(-c2nc(-n3c4ccccc4c4ccc5c6ccccc6sc5c43)nc3ccccc23)cc1] 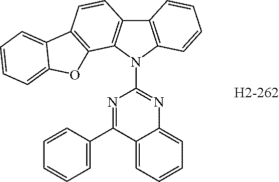 [SMILES: c1ccc(-c2nc(-n3c4ccccc4c4ccc5c6ccccc6oc5c43)nc3ccccc23)cc1] 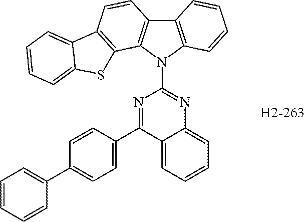 [SMILES: c1ccc(-c2ccc(-c3nc(-n4c5ccccc5c5ccc6c7ccccc7sc6c54)nc4ccccc34)cc2)cc1] 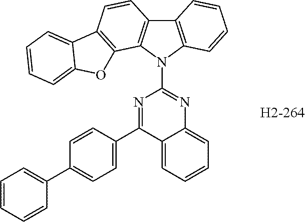 [SMILES: c1ccc(-c2ccc(-c3nc(-n4c5ccccc5c5ccc6c7ccccc7oc6c54)nc4ccccc34)cc2)cc1] 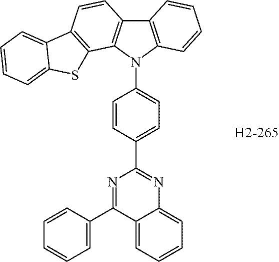 [SMILES: c1ccc(-c2nc(-c3ccc(-n4c5ccccc5c5ccc6c7ccccc7sc6c54)cc3)nc3ccccc23)cc1] 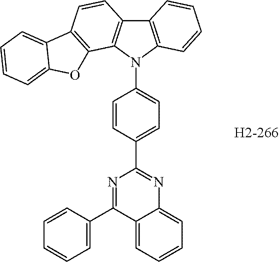 [SMILES: c1ccc(-c2nc(-c3ccc(-n4c5ccccc5c5ccc6c7ccccc7oc6c54)cc3)nc3ccccc23)cc1] 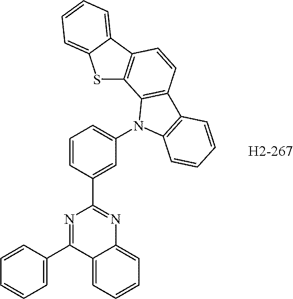 [SMILES: c1ccc(-c2nc(-c3cccc(-n4c5ccccc5c5ccc6c7ccccc7sc6c54)c3)nc3ccccc23)cc1] 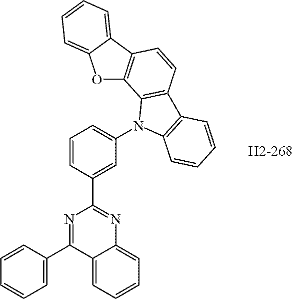 [SMILES: c1ccc(-c2nc(-c3cccc(-n4c5ccccc5c5ccc6c7ccccc7oc6c54)c3)nc3ccccc23)cc1] 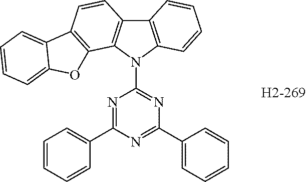 [SMILES: c1ccc(-c2nc(-c3ccccc3)nc(-n3c4ccccc4c4ccc5c6ccccc6oc5c43)n2)cc1] 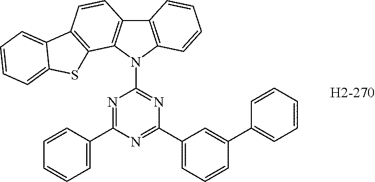 [SMILES: c1ccc(-c2cccc(-c3nc(-c4ccccc4)nc(-n4c5ccccc5c5ccc6c7ccccc7sc6c54)n3)c2)cc1] 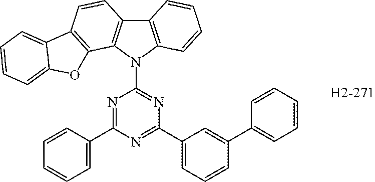 [SMILES: c1ccc(-c2cccc(-c3nc(-c4ccccc4)nc(-n4c5ccccc5c5ccc6c7ccccc7oc6c54)n3)c2)cc1] 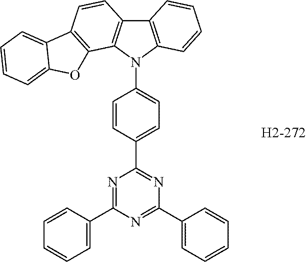 [SMILES: c1ccc(-c2nc(-c3ccccc3)nc(-c3ccc(-n4c5ccccc5c5ccc6c7ccccc7oc6c54)cc3)n2)cc1] 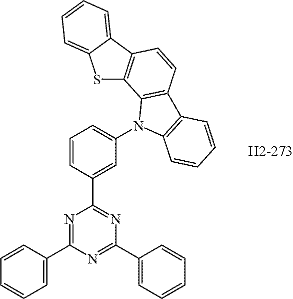 [SMILES: c1ccc(-c2nc(-c3ccccc3)nc(-c3cccc(-n4c5ccccc5c5ccc6c7ccccc7sc6c54)c3)n2)cc1] 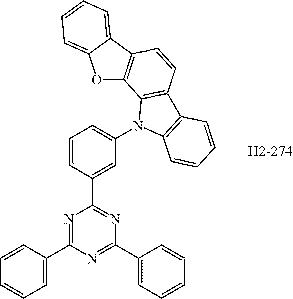 [SMILES: c1ccc(-c2nc(-c3ccccc3)nc(-c3cccc(-n4c5ccccc5c5ccc6c7ccccc7oc6c54)c3)n2)cc1] 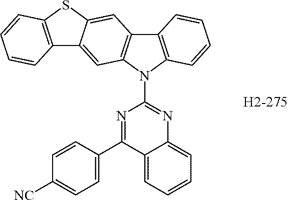 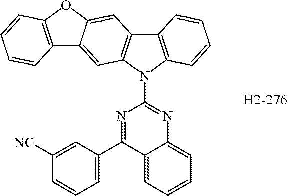 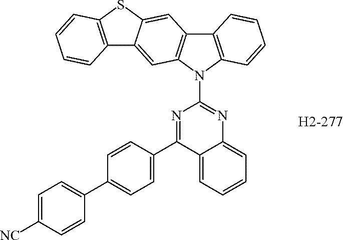 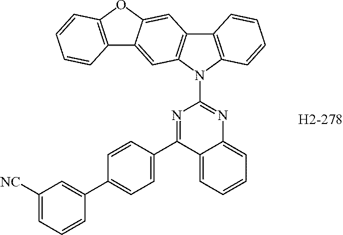 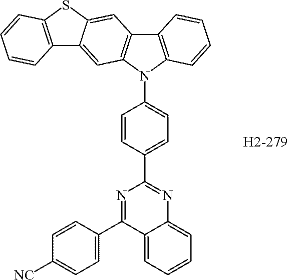 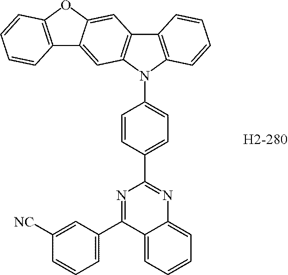 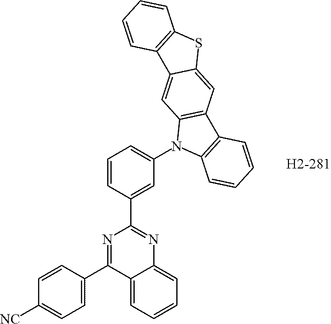 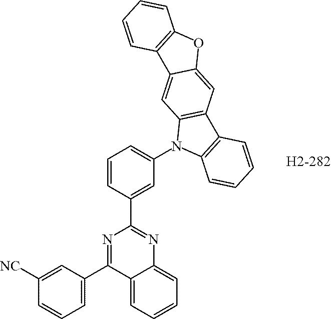 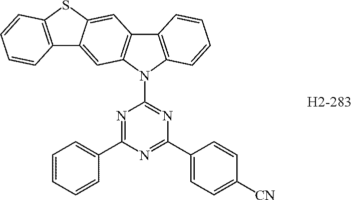 [SMILES: N#Cc1ccc(-c2nc(-c3ccccc3)nc(-n3c4ccccc4c4cc5sc6ccccc6c5cc43)n2)cc1] 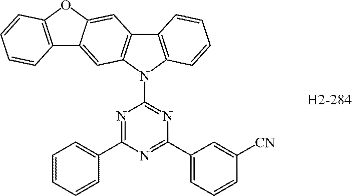 [SMILES: N#Cc1cccc(-c2nc(-c3ccccc3)nc(-n3c4ccccc4c4cc5oc6ccccc6c5cc43)n2)c1] 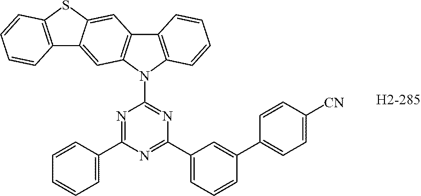 [SMILES: N#Cc1ccc(-c2cccc(-c3nc(-c4ccccc4)nc(-n4c5ccccc5c5cc6sc7ccccc7c6cc54)n3)c2)cc1] 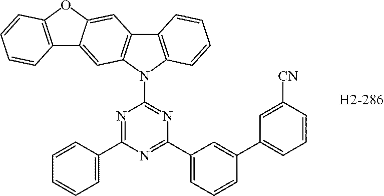 [SMILES: N#Cc1cccc(-c2cccc(-c3nc(-c4ccccc4)nc(-n4c5ccccc5c5cc6oc7ccccc7c6cc54)n3)c2)c1] 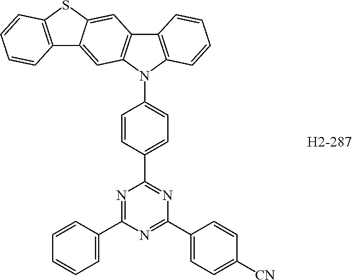 [SMILES: N#Cc1ccc(-c2nc(-c3ccccc3)nc(-c3ccc(-n4c5ccccc5c5cc6sc7ccccc7c6cc54)cc3)n2)cc1] 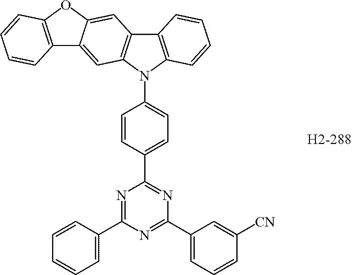 [SMILES: N#Cc1cccc(-c2nc(-c3ccccc3)nc(-c3ccc(-n4c5ccccc5c5cc6oc7ccccc7c6cc54)cc3)n2)c1] 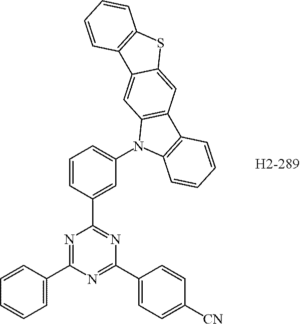 [SMILES: N#Cc1ccc(-c2nc(-c3ccccc3)nc(-c3cccc(-n4c5ccccc5c5cc6sc7ccccc7c6cc54)c3)n2)cc1] 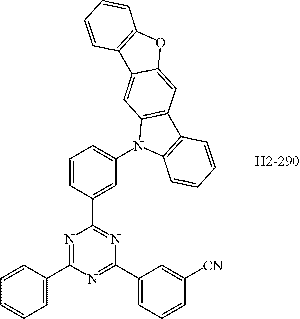 [SMILES: N#Cc1cccc(-c2nc(-c3ccccc3)nc(-c3cccc(-n4c5ccccc5c5cc6oc7ccccc7c6cc54)c3)n2)c1] 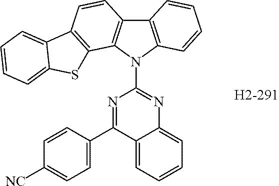 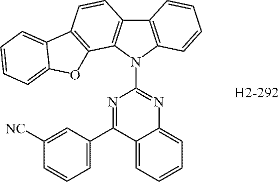 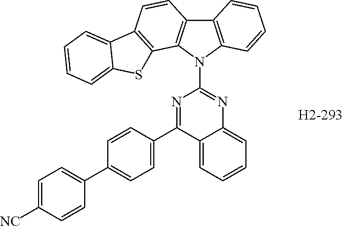 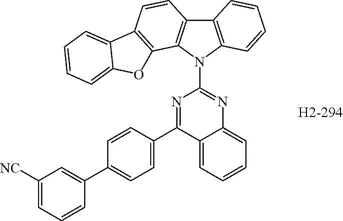 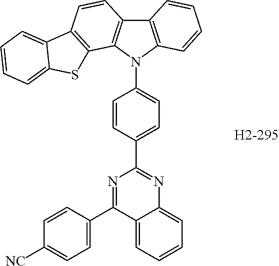 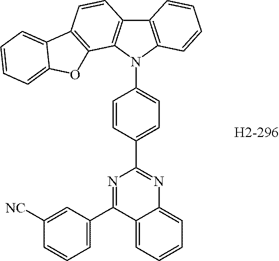 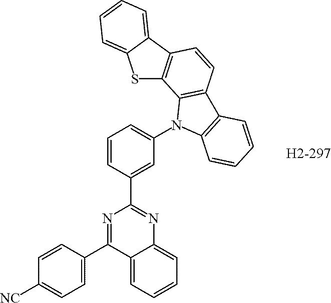 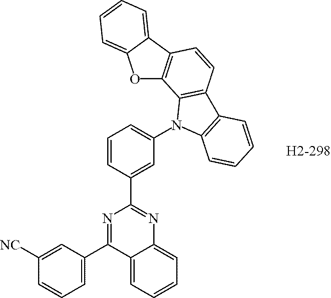 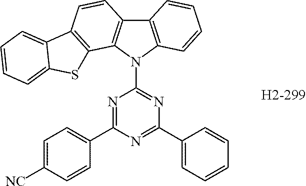 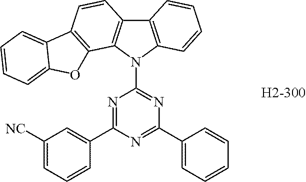 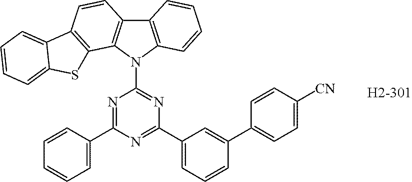 [SMILES: N#Cc1ccc(-c2cccc(-c3nc(-c4ccccc4)nc(-n4c5ccccc5c5ccc6c7ccccc7sc6c54)n3)c2)cc1] 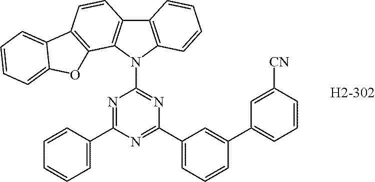 [SMILES: N#Cc1cccc(-c2cccc(-c3nc(-c4ccccc4)nc(-n4c5ccccc5c5ccc6c7ccccc7oc6c54)n3)c2)c1] 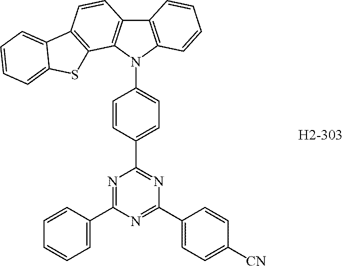 [SMILES: N#Cc1ccc(-c2nc(-c3ccccc3)nc(-c3ccc(-n4c5ccccc5c5ccc6c7ccccc7sc6c54)cc3)n2)cc1] 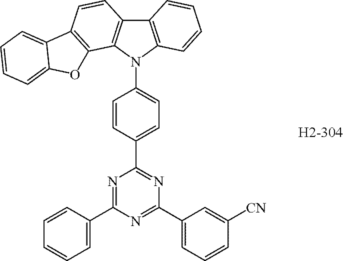 [SMILES: N#Cc1cccc(-c2nc(-c3ccccc3)nc(-c3ccc(-n4c5ccccc5c5ccc6c7ccccc7oc6c54)cc3)n2)c1] 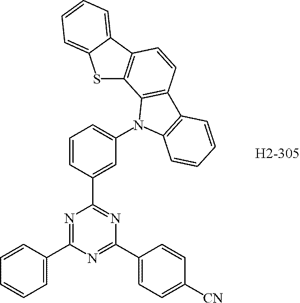 [SMILES: N#Cc1ccc(-c2nc(-c3ccccc3)nc(-c3cccc(-n4c5ccccc5c5ccc6c7ccccc7sc6c54)c3)n2)cc1] 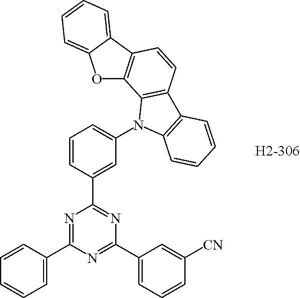 [SMILES: N#Cc1cccc(-c2nc(-c3ccccc3)nc(-c3cccc(-n4c5ccccc5c5ccc6c7ccccc7oc6c54)c3)n2)c1] 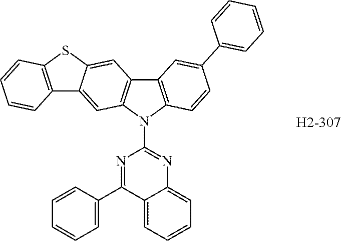 [SMILES: c1ccc(-c2ccc3c(c2)c2cc4sc5ccccc5c4cc2n3-c2nc(-c3ccccc3)c3ccccc3n2)cc1] 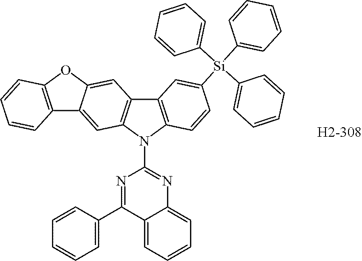 [SMILES: c1ccc(-c2nc(-n3c4ccc([Si](c5ccccc5)(c5ccccc5)c5ccccc5)cc4c4cc5oc6ccccc6c5cc43)nc3ccccc23)cc1] 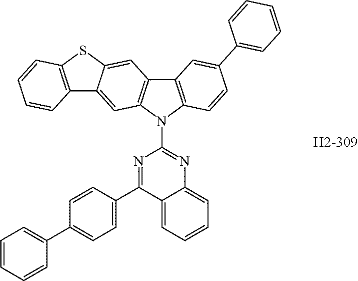 [SMILES: c1ccc(-c2ccc(-c3nc(-n4c5ccc(-c6ccccc6)cc5c5cc6sc7ccccc7c6cc54)nc4ccccc34)cc2)cc1] 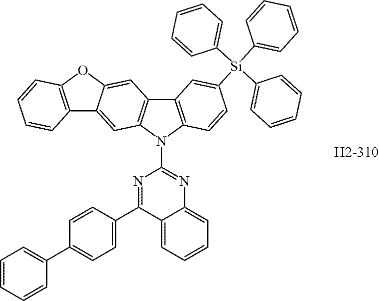 [SMILES: c1ccc(-c2ccc(-c3nc(-n4c5ccc([Si](c6ccccc6)(c6ccccc6)c6ccccc6)cc5c5cc6oc7ccccc7c6cc54)nc4ccccc34)cc2)cc1] 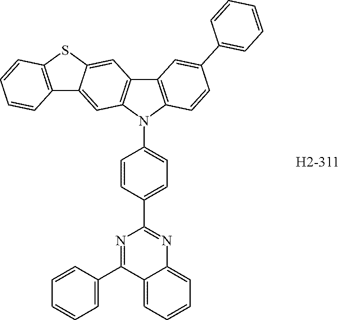 [SMILES: c1ccc(-c2ccc3c(c2)c2cc4sc5ccccc5c4cc2n3-c2ccc(-c3nc(-c4ccccc4)c4ccccc4n3)cc2)cc1] 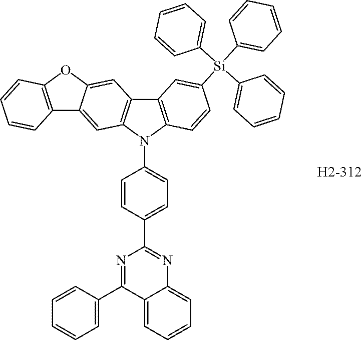 [SMILES: c1ccc(-c2nc(-c3ccc(-n4c5ccc([Si](c6ccccc6)(c6ccccc6)c6ccccc6)cc5c5cc6oc7ccccc7c6cc54)cc3)nc3ccccc23)cc1] 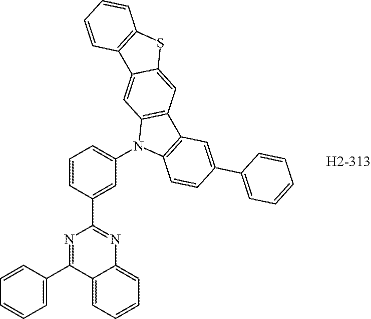 [SMILES: c1ccc(-c2ccc3c(c2)c2cc4sc5ccccc5c4cc2n3-c2cccc(-c3nc(-c4ccccc4)c4ccccc4n3)c2)cc1] 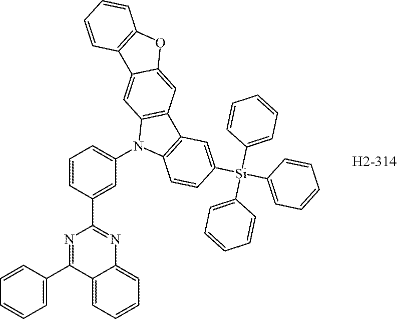 [SMILES: c1ccc(-c2nc(-c3cccc(-n4c5ccc([Si](c6ccccc6)(c6ccccc6)c6ccccc6)cc5c5cc6oc7ccccc7c6cc54)c3)nc3ccccc23)cc1] 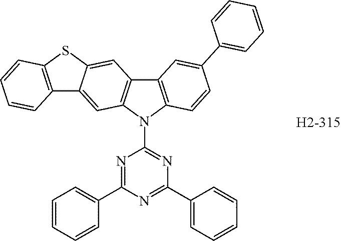 [SMILES: c1ccc(-c2ccc3c(c2)c2cc4sc5ccccc5c4cc2n3-c2nc(-c3ccccc3)nc(-c3ccccc3)n2)cc1] 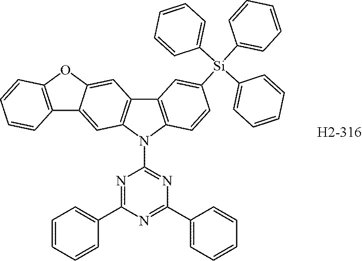 [SMILES: c1ccc(-c2nc(-c3ccccc3)nc(-n3c4ccc([Si](c5ccccc5)(c5ccccc5)c5ccccc5)cc4c4cc5oc6ccccc6c5cc43)n2)cc1] 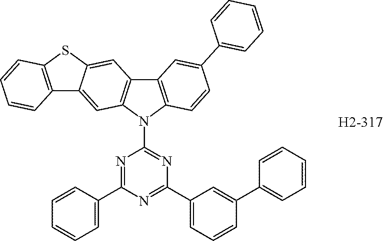 [SMILES: c1ccc(-c2cccc(-c3nc(-c4ccccc4)nc(-n4c5ccc(-c6ccccc6)cc5c5cc6sc7ccccc7c6cc54)n3)c2)cc1] 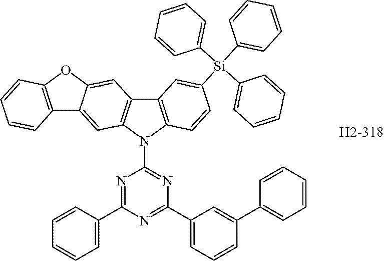 [SMILES: c1ccc(-c2cccc(-c3nc(-c4ccccc4)nc(-n4c5ccc([Si](c6ccccc6)(c6ccccc6)c6ccccc6)cc5c5cc6oc7ccccc7c6cc54)n3)c2)cc1] 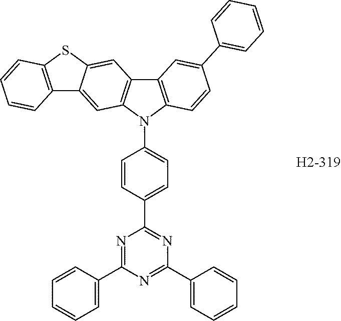 [SMILES: c1ccc(-c2ccc3c(c2)c2cc4sc5ccccc5c4cc2n3-c2ccc(-c3nc(-c4ccccc4)nc(-c4ccccc4)n3)cc2)cc1] 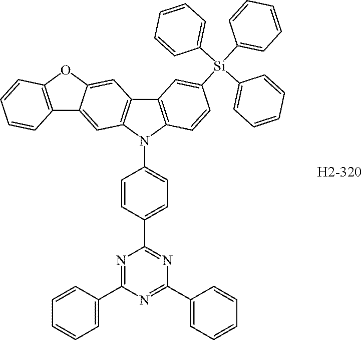 [SMILES: c1ccc(-c2nc(-c3ccccc3)nc(-c3ccc(-n4c5ccc([Si](c6ccccc6)(c6ccccc6)c6ccccc6)cc5c5cc6oc7ccccc7c6cc54)cc3)n2)cc1] 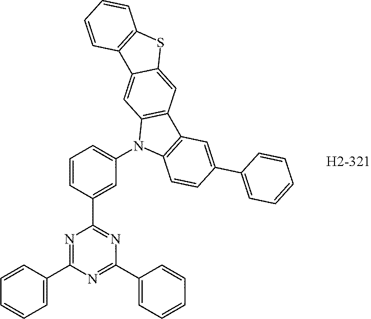 [SMILES: c1ccc(-c2ccc3c(c2)c2cc4sc5ccccc5c4cc2n3-c2cccc(-c3nc(-c4ccccc4)nc(-c4ccccc4)n3)c2)cc1] 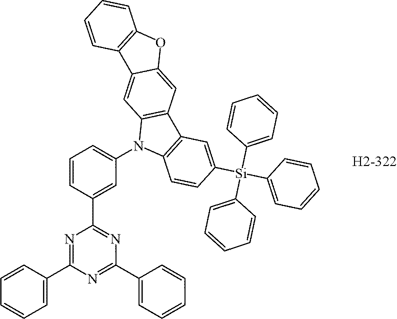 [SMILES: c1ccc(-c2nc(-c3ccccc3)nc(-c3cccc(-n4c5ccc([Si](c6ccccc6)(c6ccccc6)c6ccccc6)cc5c5cc6oc7ccccc7c6cc54)c3)n2)cc1] 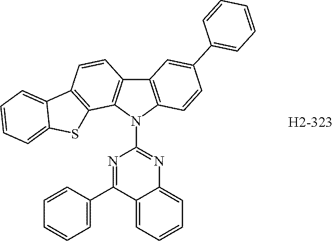 [SMILES: c1ccc(-c2ccc3c(c2)c2ccc4c5ccccc5sc4c2n3-c2nc(-c3ccccc3)c3ccccc3n2)cc1] 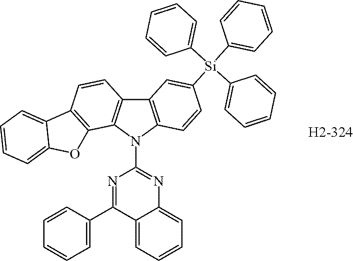 [SMILES: c1ccc(-c2nc(-n3c4ccc([Si](c5ccccc5)(c5ccccc5)c5ccccc5)cc4c4ccc5c6ccccc6oc5c43)nc3ccccc23)cc1] 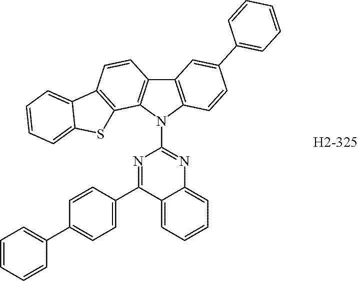 [SMILES: c1ccc(-c2ccc(-c3nc(-n4c5ccc(-c6ccccc6)cc5c5ccc6c7ccccc7sc6c54)nc4ccccc34)cc2)cc1] 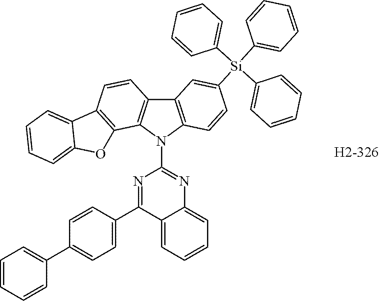 [SMILES: c1ccc(-c2ccc(-c3nc(-n4c5ccc([Si](c6ccccc6)(c6ccccc6)c6ccccc6)cc5c5ccc6c7ccccc7oc6c54)nc4ccccc34)cc2)cc1] 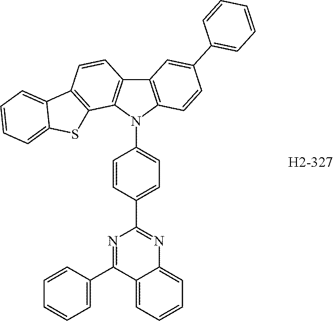 [SMILES: c1ccc(-c2ccc3c(c2)c2ccc4c5ccccc5sc4c2n3-c2ccc(-c3nc(-c4ccccc4)c4ccccc4n3)cc2)cc1] 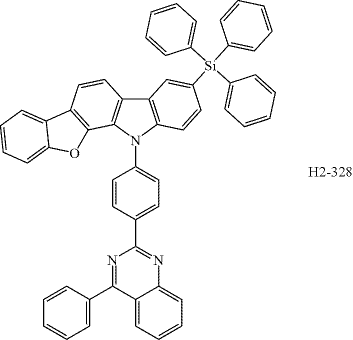 [SMILES: c1ccc(-c2nc(-c3ccc(-n4c5ccc([Si](c6ccccc6)(c6ccccc6)c6ccccc6)cc5c5ccc6c7ccccc7oc6c54)cc3)nc3ccccc23)cc1] 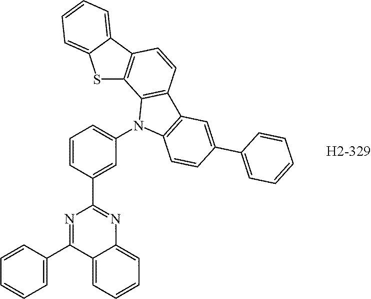 [SMILES: c1ccc(-c2ccc3c(c2)c2ccc4c5ccccc5sc4c2n3-c2cccc(-c3nc(-c4ccccc4)c4ccccc4n3)c2)cc1] 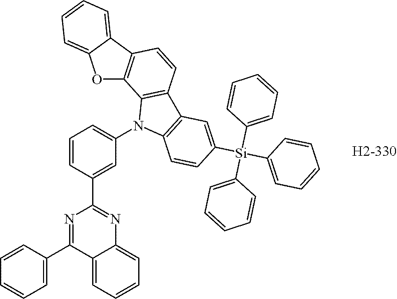 [SMILES: c1ccc(-c2nc(-c3cccc(-n4c5ccc([Si](c6ccccc6)(c6ccccc6)c6ccccc6)cc5c5ccc6c7ccccc7oc6c54)c3)nc3ccccc23)cc1] 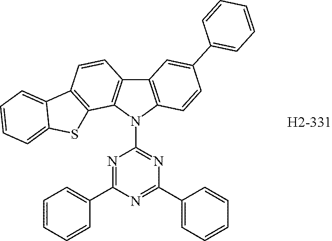 [SMILES: c1ccc(-c2ccc3c(c2)c2ccc4c5ccccc5sc4c2n3-c2nc(-c3ccccc3)nc(-c3ccccc3)n2)cc1] 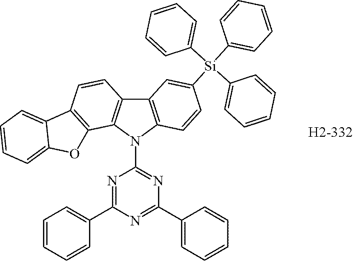 [SMILES: c1ccc(-c2nc(-c3ccccc3)nc(-n3c4ccc([Si](c5ccccc5)(c5ccccc5)c5ccccc5)cc4c4ccc5c6ccccc6oc5c43)n2)cc1] 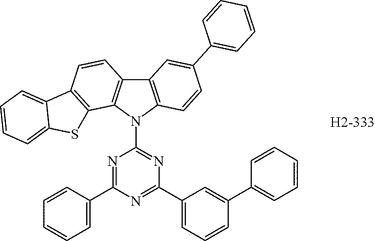 [SMILES: c1ccc(-c2cccc(-c3nc(-c4ccccc4)nc(-n4c5ccc(-c6ccccc6)cc5c5ccc6c7ccccc7sc6c54)n3)c2)cc1] 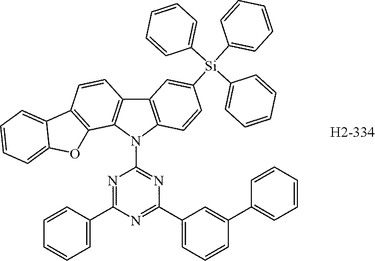 [SMILES: c1ccc(-c2cccc(-c3nc(-c4ccccc4)nc(-n4c5ccc([Si](c6ccccc6)(c6ccccc6)c6ccccc6)cc5c5ccc6c7ccccc7oc6c54)n3)c2)cc1] 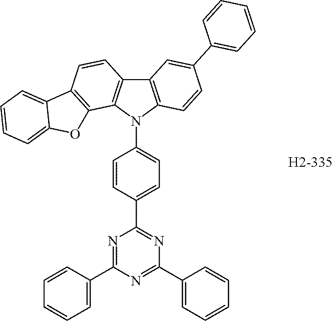 [SMILES: c1ccc(-c2ccc3c(c2)c2ccc4c5ccccc5oc4c2n3-c2ccc(-c3nc(-c4ccccc4)nc(-c4ccccc4)n3)cc2)cc1] 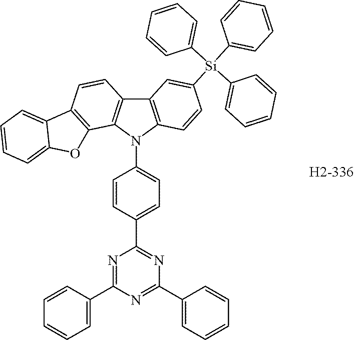 [SMILES: c1ccc(-c2nc(-c3ccccc3)nc(-c3ccc(-n4c5ccc([Si](c6ccccc6)(c6ccccc6)c6ccccc6)cc5c5ccc6c7ccccc7oc6c54)cc3)n2)cc1] 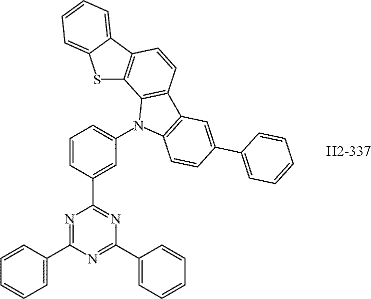 [SMILES: c1ccc(-c2ccc3c(c2)c2ccc4c5ccccc5sc4c2n3-c2cccc(-c3nc(-c4ccccc4)nc(-c4ccccc4)n3)c2)cc1] 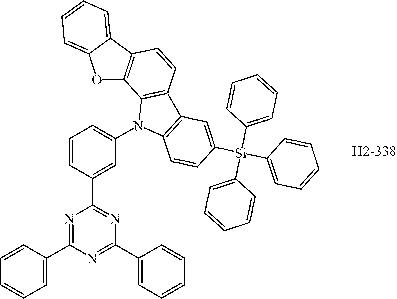 [SMILES: c1ccc(-c2nc(-c3ccccc3)nc(-c3cccc(-n4c5ccc([Si](c6ccccc6)(c6ccccc6)c6ccccc6)cc5c5ccc6c7ccccc7oc6c54)c3)n2)cc1] 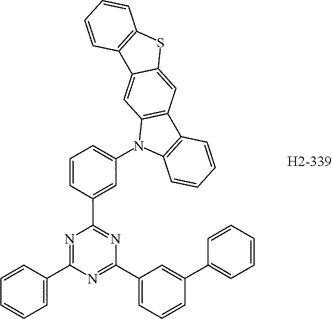 [SMILES: c1ccc(-c2cccc(-c3nc(-c4ccccc4)nc(-c4cccc(-n5c6ccccc6c6cc7sc8ccccc8c7cc65)c4)n3)c2)cc1] 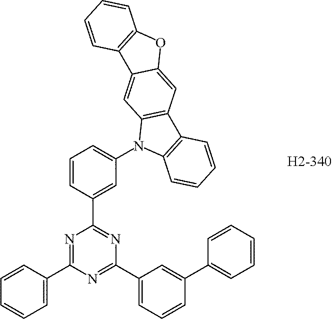 [SMILES: c1ccc(-c2cccc(-c3nc(-c4ccccc4)nc(-c4cccc(-n5c6ccccc6c6cc7oc8ccccc8c7cc65)c4)n3)c2)cc1] 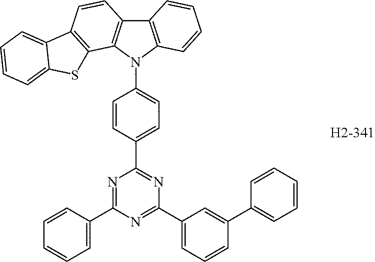 [SMILES: c1ccc(-c2cccc(-c3nc(-c4ccccc4)nc(-c4ccc(-n5c6ccccc6c6ccc7c8ccccc8sc7c65)cc4)n3)c2)cc1] 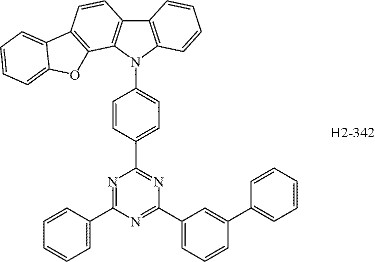 [SMILES: c1ccc(-c2cccc(-c3nc(-c4ccccc4)nc(-c4ccc(-n5c6ccccc6c6ccc7c8ccccc8oc7c65)cc4)n3)c2)cc1] 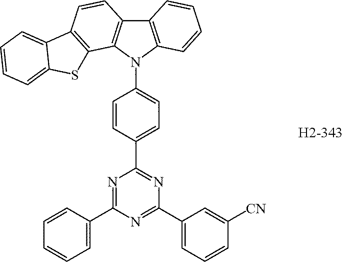 [SMILES: N#Cc1cccc(-c2nc(-c3ccccc3)nc(-c3ccc(-n4c5ccccc5c5ccc6c7ccccc7sc6c54)cc3)n2)c1] 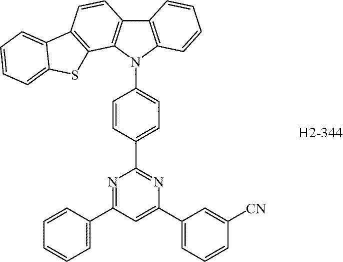 [SMILES: N#Cc1cccc(-c2cc(-c3ccccc3)nc(-c3ccc(-n4c5ccccc5c5ccc6c7ccccc7sc6c54)cc3)n2)c1] 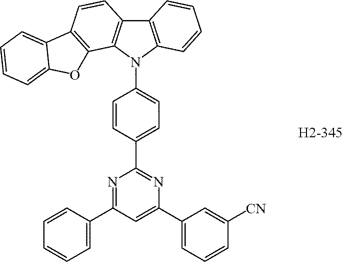 [SMILES: N#Cc1cccc(-c2cc(-c3ccccc3)nc(-c3ccc(-n4c5ccccc5c5ccc6c7ccccc7oc6c54)cc3)n2)c1] 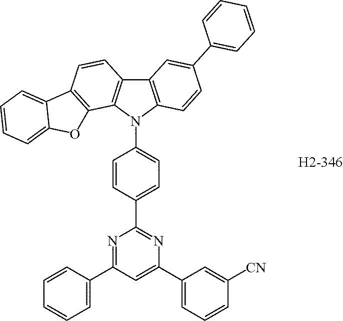 [SMILES: N#Cc1cccc(-c2cc(-c3ccccc3)nc(-c3ccc(-n4c5ccc(-c6ccccc6)cc5c5ccc6c7ccccc7oc6c54)cc3)n2)c1] 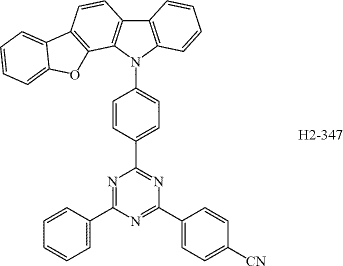 [SMILES: N#Cc1ccc(-c2nc(-c3ccccc3)nc(-c3ccc(-n4c5ccccc5c5ccc6c7ccccc7oc6c54)cc3)n2)cc1] 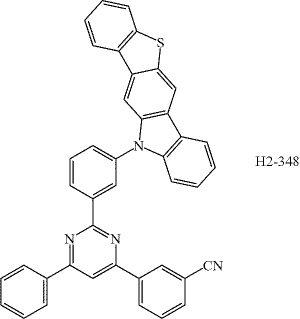 [SMILES: N#Cc1cccc(-c2cc(-c3ccccc3)nc(-c3cccc(-n4c5ccccc5c5cc6sc7ccccc7c6cc54)c3)n2)c1] 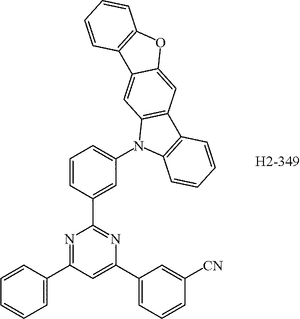 [SMILES: N#Cc1cccc(-c2cc(-c3ccccc3)nc(-c3cccc(-n4c5ccccc5c5cc6oc7ccccc7c6cc54)c3)n2)c1] 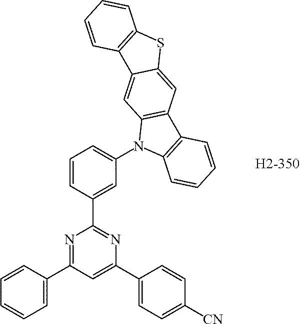 [SMILES: N#Cc1ccc(-c2cc(-c3ccccc3)nc(-c3cccc(-n4c5ccccc5c5cc6sc7ccccc7c6cc54)c3)n2)cc1] 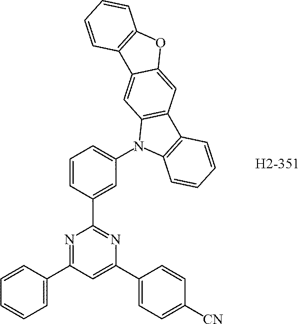 [SMILES: N#Cc1ccc(-c2cc(-c3ccccc3)nc(-c3cccc(-n4c5ccccc5c5cc6oc7ccccc7c6cc54)c3)n2)cc1] 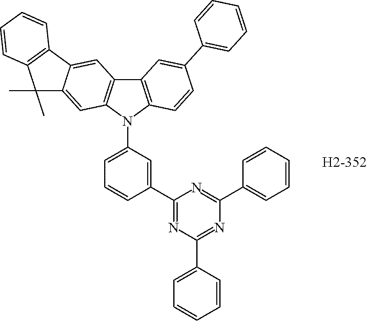 [SMILES: CC1(C)c2ccccc2-c2cc3c4cc(-c5ccccc5)ccc4n(-c4cccc(-c5nc(-c6ccccc6)nc(-c6ccccc6)n5)c4)c3cc21] 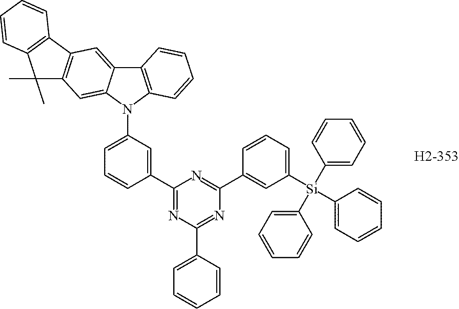 [SMILES: CC1(C)c2ccccc2-c2cc3c4ccccc4n(-c4cccc(-c5nc(-c6ccccc6)nc(-c6cccc([Si](c7ccccc7)(c7ccccc7)c7ccccc7)c6)n5)c4)c3cc21] 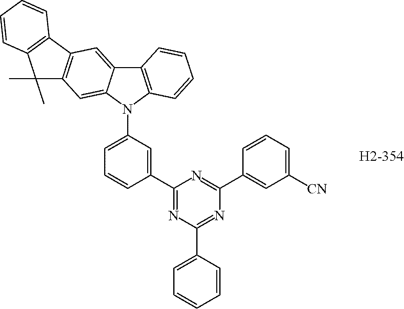 [SMILES: CC1(C)c2ccccc2-c2cc3c4ccccc4n(-c4cccc(-c5nc(-c6ccccc6)nc(-c6cccc(C#N)c6)n5)c4)c3cc21] 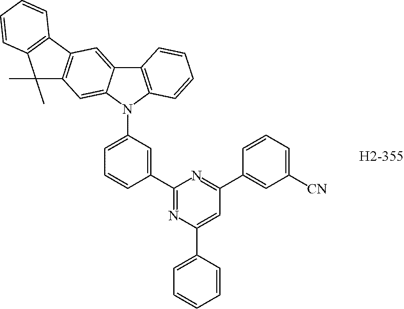 [SMILES: CC1(C)c2ccccc2-c2cc3c4ccccc4n(-c4cccc(-c5nc(-c6ccccc6)cc(-c6cccc(C#N)c6)n5)c4)c3cc21] 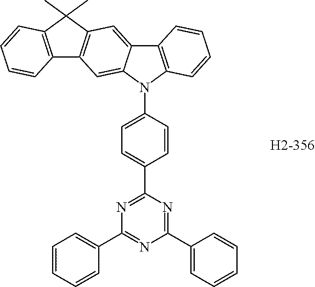 [SMILES: CC1(C)c2ccccc2-c2cc3c(cc21)c1ccccc1n3-c1ccc(-c2nc(-c3ccccc3)nc(-c3ccccc3)n2)cc1] 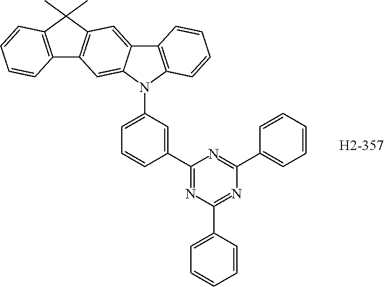 [SMILES: CC1(C)c2ccccc2-c2cc3c(cc21)c1ccccc1n3-c1cccc(-c2nc(-c3ccccc3)nc(-c3ccccc3)n2)c1] 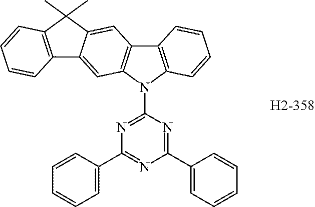 [SMILES: CC1(C)c2ccccc2-c2cc3c(cc21)c1ccccc1n3-c1nc(-c2ccccc2)nc(-c2ccccc2)n1] 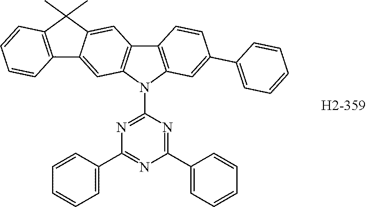 [SMILES: CC1(C)c2ccccc2-c2cc3c(cc21)c1ccc(-c2ccccc2)cc1n3-c1nc(-c2ccccc2)nc(-c2ccccc2)n1] 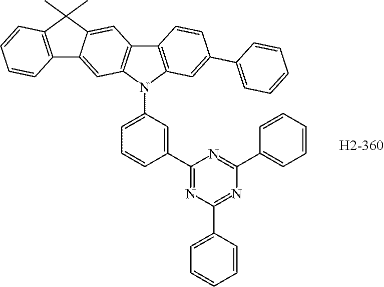 [SMILES: CC1(C)c2ccccc2-c2cc3c(cc21)c1ccc(-c2ccccc2)cc1n3-c1cccc(-c2nc(-c3ccccc3)nc(-c3ccccc3)n2)c1] 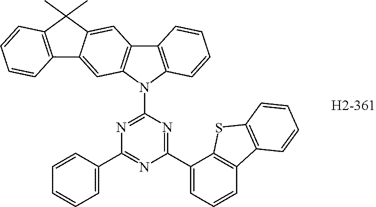 [SMILES: CC1(C)c2ccccc2-c2cc3c(cc21)c1ccccc1n3-c1nc(-c2ccccc2)nc(-c2cccc3c2sc2ccccc23)n1] 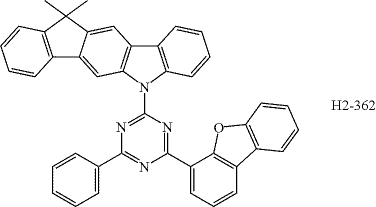 [SMILES: CC1(C)c2ccccc2-c2cc3c(cc21)c1ccccc1n3-c1nc(-c2ccccc2)nc(-c2cccc3c2oc2ccccc23)n1] 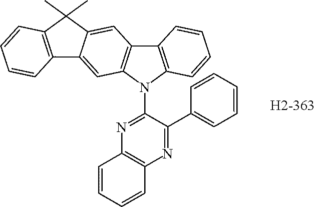 [SMILES: CC1(C)c2ccccc2-c2cc3c(cc21)c1ccccc1n3-c1nc2ccccc2nc1-c1ccccc1] 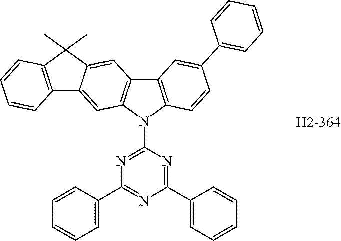 [SMILES: CC1(C)c2ccccc2-c2cc3c(cc21)c1cc(-c2ccccc2)ccc1n3-c1nc(-c2ccccc2)nc(-c2ccccc2)n1] 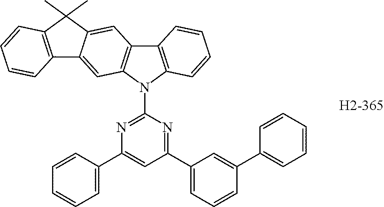 [SMILES: CC1(C)c2ccccc2-c2cc3c(cc21)c1ccccc1n3-c1nc(-c2ccccc2)cc(-c2cccc(-c3ccccc3)c2)n1] 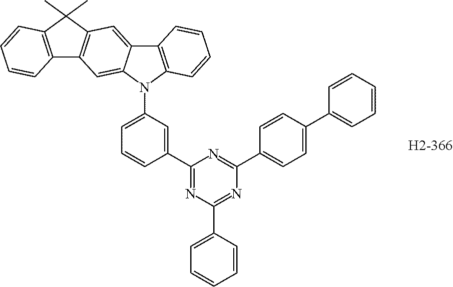 [SMILES: CC1(C)c2ccccc2-c2cc3c(cc21)c1ccccc1n3-c1cccc(-c2nc(-c3ccccc3)nc(-c3ccc(-c4ccccc4)cc3)n2)c1] 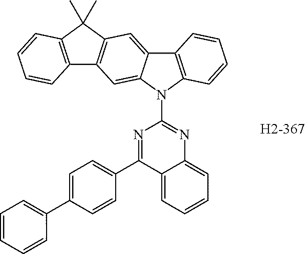 [SMILES: CC1(C)c2ccccc2-c2cc3c(cc21)c1ccccc1n3-c1nc(-c2ccc(-c3ccccc3)cc2)c2ccccc2n1] 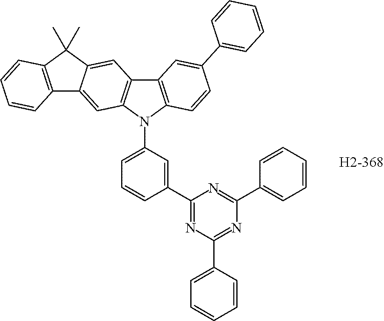 [SMILES: CC1(C)c2ccccc2-c2cc3c(cc21)c1cc(-c2ccccc2)ccc1n3-c1cccc(-c2nc(-c3ccccc3)nc(-c3ccccc3)n2)c1] 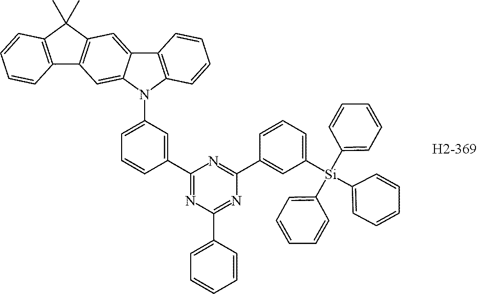 [SMILES: CC1(C)c2ccccc2-c2cc3c(cc21)c1ccccc1n3-c1cccc(-c2nc(-c3ccccc3)nc(-c3cccc([Si](c4ccccc4)(c4ccccc4)c4ccccc4)c3)n2)c1] 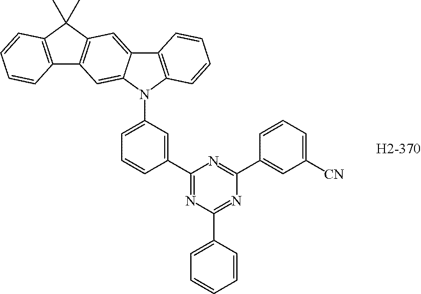 [SMILES: CC1(C)c2ccccc2-c2cc3c(cc21)c1ccccc1n3-c1cccc(-c2nc(-c3ccccc3)nc(-c3cccc(C#N)c3)n2)c1] 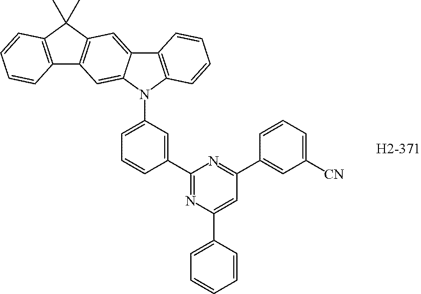 [SMILES: CC1(C)c2ccccc2-c2cc3c(cc21)c1ccccc1n3-c1cccc(-c2nc(-c3ccccc3)cc(-c3cccc(C#N)c3)n2)c1] 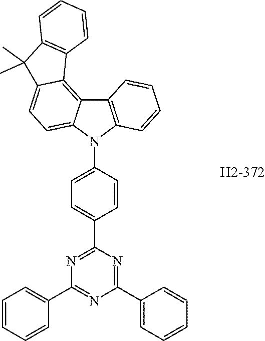 [SMILES: CC1(C)c2ccccc2-c2c1ccc1c2c2ccccc2n1-c1ccc(-c2nc(-c3ccccc3)nc(-c3ccccc3)n2)cc1] 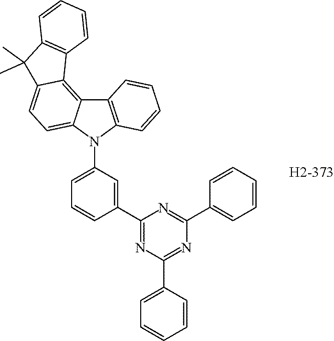 [SMILES: CC1(C)c2ccccc2-c2c1ccc1c2c2ccccc2n1-c1cccc(-c2nc(-c3ccccc3)nc(-c3ccccc3)n2)c1] 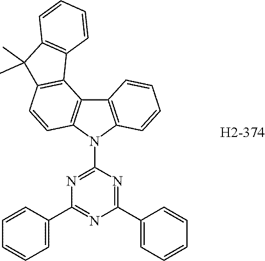 [SMILES: CC1(C)c2ccccc2-c2c1ccc1c2c2ccccc2n1-c1nc(-c2ccccc2)nc(-c2ccccc2)n1] 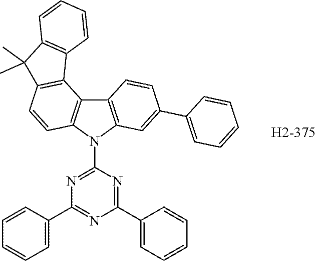 [SMILES: CC1(C)c2ccccc2-c2c1ccc1c2c2ccc(-c3ccccc3)cc2n1-c1nc(-c2ccccc2)nc(-c2ccccc2)n1] 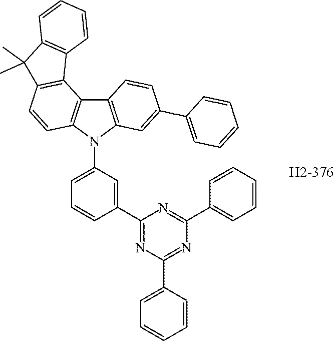 [SMILES: CC1(C)c2ccccc2-c2c1ccc1c2c2ccc(-c3ccccc3)cc2n1-c1cccc(-c2nc(-c3ccccc3)nc(-c3ccccc3)n2)c1] 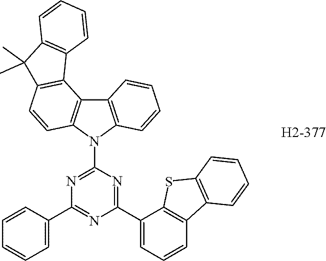 [SMILES: CC1(C)c2ccccc2-c2c1ccc1c2c2ccccc2n1-c1nc(-c2ccccc2)nc(-c2cccc3c2sc2ccccc23)n1] 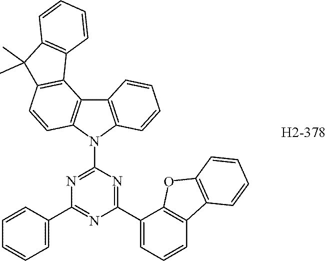 [SMILES: CC1(C)c2ccccc2-c2c1ccc1c2c2ccccc2n1-c1nc(-c2ccccc2)nc(-c2cccc3c2oc2ccccc23)n1] 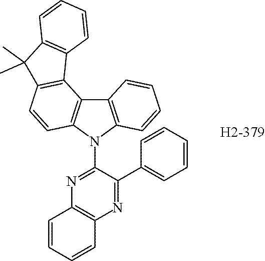 [SMILES: CC1(C)c2ccccc2-c2c1ccc1c2c2ccccc2n1-c1nc2ccccc2nc1-c1ccccc1] 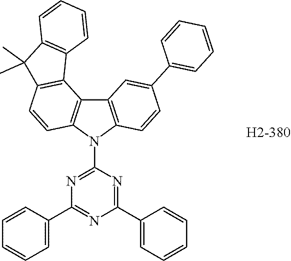 [SMILES: CC1(C)c2ccccc2-c2c1ccc1c2c2cc(-c3ccccc3)ccc2n1-c1nc(-c2ccccc2)nc(-c2ccccc2)n1] 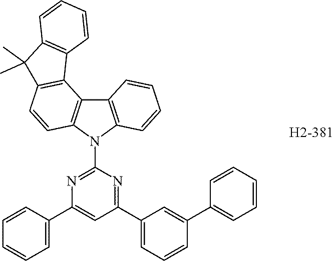 [SMILES: CC1(C)c2ccccc2-c2c1ccc1c2c2ccccc2n1-c1nc(-c2ccccc2)cc(-c2cccc(-c3ccccc3)c2)n1] 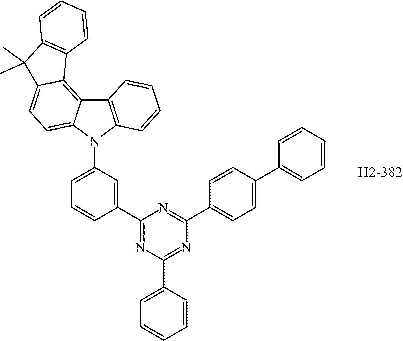 [SMILES: CC1(C)c2ccccc2-c2c1ccc1c2c2ccccc2n1-c1cccc(-c2nc(-c3ccccc3)nc(-c3ccc(-c4ccccc4)cc3)n2)c1] 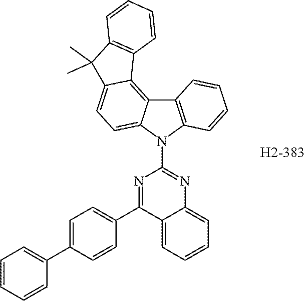 [SMILES: CC1(C)c2ccccc2-c2c1ccc1c2c2ccccc2n1-c1nc(-c2ccc(-c3ccccc3)cc2)c2ccccc2n1] 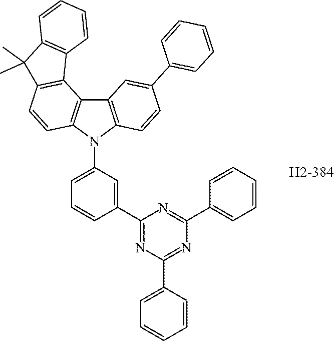 [SMILES: CC1(C)c2ccccc2-c2c1ccc1c2c2cc(-c3ccccc3)ccc2n1-c1cccc(-c2nc(-c3ccccc3)nc(-c3ccccc3)n2)c1] 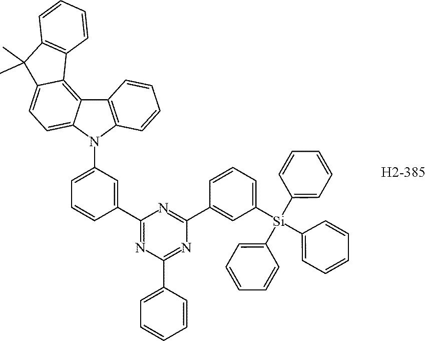 [SMILES: CC1(C)c2ccccc2-c2c1ccc1c2c2ccccc2n1-c1cccc(-c2nc(-c3ccccc3)nc(-c3cccc([Si](c4ccccc4)(c4ccccc4)c4ccccc4)c3)n2)c1] 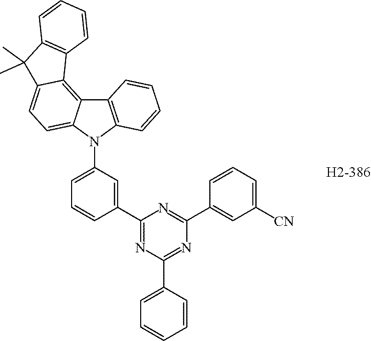 [SMILES: CC1(C)c2ccccc2-c2c1ccc1c2c2ccccc2n1-c1cccc(-c2nc(-c3ccccc3)nc(-c3cccc(C#N)c3)n2)c1] 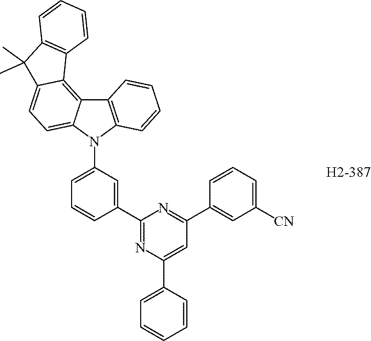 [SMILES: CC1(C)c2ccccc2-c2c1ccc1c2c2ccccc2n1-c1cccc(-c2nc(-c3ccccc3)cc(-c3cccc(C#N)c3)n2)c1] 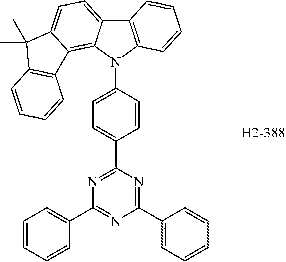 [SMILES: CC1(C)c2ccccc2-c2c1ccc1c3ccccc3n(-c3ccc(-c4nc(-c5ccccc5)nc(-c5ccccc5)n4)cc3)c21] 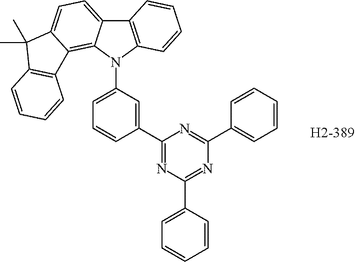 [SMILES: CC1(C)c2ccccc2-c2c1ccc1c3ccccc3n(-c3cccc(-c4nc(-c5ccccc5)nc(-c5ccccc5)n4)c3)c21] 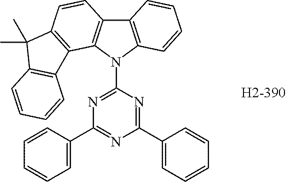 [SMILES: CC1(C)c2ccccc2-c2c1ccc1c3ccccc3n(-c3nc(-c4ccccc4)nc(-c4ccccc4)n3)c21] 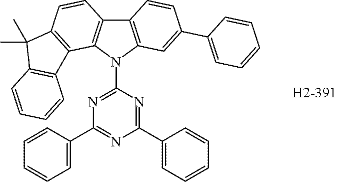 [SMILES: CC1(C)c2ccccc2-c2c1ccc1c3ccc(-c4ccccc4)cc3n(-c3nc(-c4ccccc4)nc(-c4ccccc4)n3)c21] 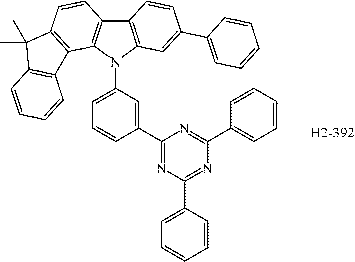 [SMILES: CC1(C)c2ccccc2-c2c1ccc1c3ccc(-c4ccccc4)cc3n(-c3cccc(-c4nc(-c5ccccc5)nc(-c5ccccc5)n4)c3)c21] 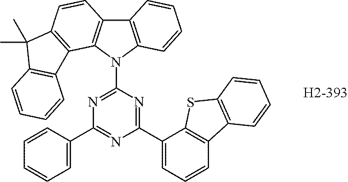 [SMILES: CC1(C)c2ccccc2-c2c1ccc1c3ccccc3n(-c3nc(-c4ccccc4)nc(-c4cccc5c4sc4ccccc45)n3)c21] 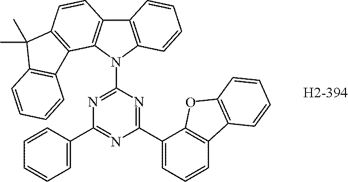 [SMILES: CC1(C)c2ccccc2-c2c1ccc1c3ccccc3n(-c3nc(-c4ccccc4)nc(-c4cccc5c4oc4ccccc45)n3)c21] 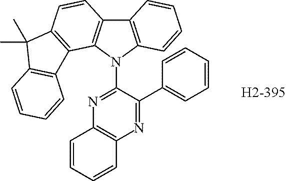 [SMILES: CC1(C)c2ccccc2-c2c1ccc1c3ccccc3n(-c3nc4ccccc4nc3-c3ccccc3)c21] 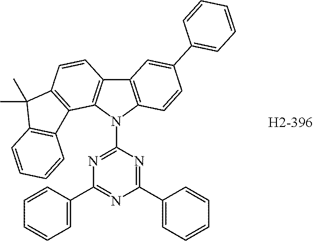 [SMILES: CC1(C)c2ccccc2-c2c1ccc1c3cc(-c4ccccc4)ccc3n(-c3nc(-c4ccccc4)nc(-c4ccccc4)n3)c21] 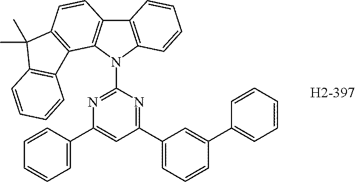 [SMILES: CC1(C)c2ccccc2-c2c1ccc1c3ccccc3n(-c3nc(-c4ccccc4)cc(-c4cccc(-c5ccccc5)c4)n3)c21] 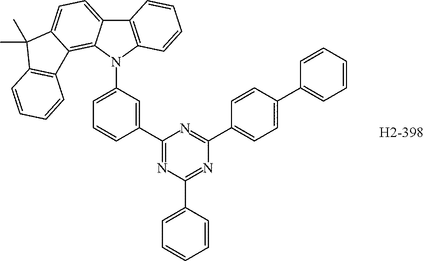 [SMILES: CC1(C)c2ccccc2-c2c1ccc1c3ccccc3n(-c3cccc(-c4nc(-c5ccccc5)nc(-c5ccc(-c6ccccc6)cc5)n4)c3)c21] 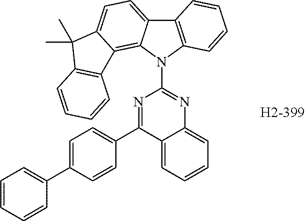 [SMILES: CC1(C)c2ccccc2-c2c1ccc1c3ccccc3n(-c3nc(-c4ccc(-c5ccccc5)cc4)c4ccccc4n3)c21] 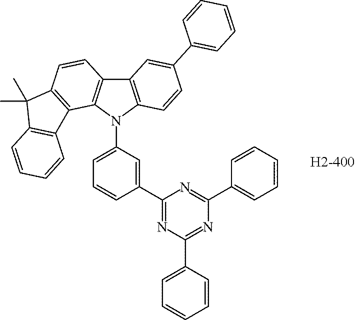 [SMILES: CC1(C)c2ccccc2-c2c1ccc1c3cc(-c4ccccc4)ccc3n(-c3cccc(-c4nc(-c5ccccc5)nc(-c5ccccc5)n4)c3)c21] 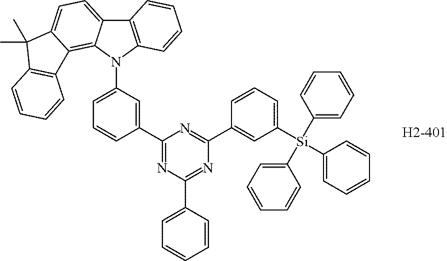 [SMILES: CC1(C)c2ccccc2-c2c1ccc1c3ccccc3n(-c3cccc(-c4nc(-c5ccccc5)nc(-c5cccc([Si](c6ccccc6)(c6ccccc6)c6ccccc6)c5)n4)c3)c21] 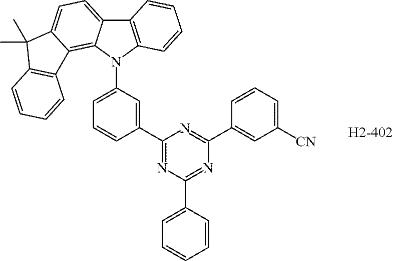 [SMILES: CC1(C)c2ccccc2-c2c1ccc1c3ccccc3n(-c3cccc(-c4nc(-c5ccccc5)nc(-c5cccc(C#N)c5)n4)c3)c21] 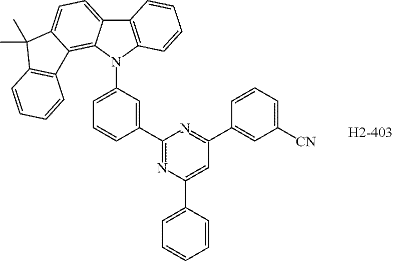 [SMILES: CC1(C)c2ccccc2-c2c1ccc1c3ccccc3n(-c3cccc(-c4nc(-c5ccccc5)cc(-c5cccc(C#N)c5)n4)c3)c21] 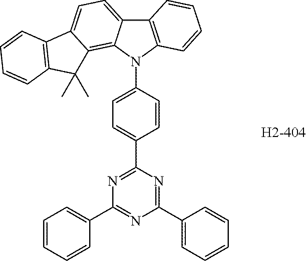 [SMILES: CC1(C)c2ccccc2-c2ccc3c4ccccc4n(-c4ccc(-c5nc(-c6ccccc6)nc(-c6ccccc6)n5)cc4)c3c21] 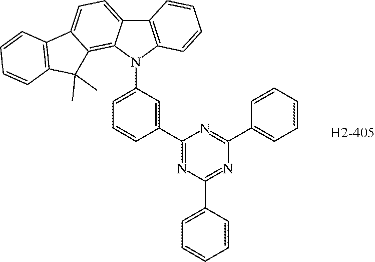 [SMILES: CC1(C)c2ccccc2-c2ccc3c4ccccc4n(-c4cccc(-c5nc(-c6ccccc6)nc(-c6ccccc6)n5)c4)c3c21] 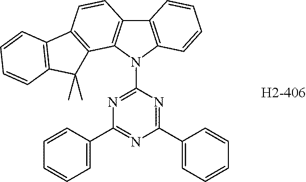 [SMILES: CC1(C)c2ccccc2-c2ccc3c4ccccc4n(-c4nc(-c5ccccc5)nc(-c5ccccc5)n4)c3c21] 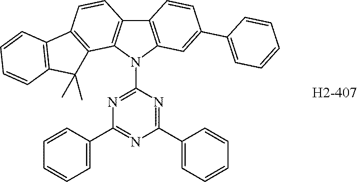 [SMILES: CC1(C)c2ccccc2-c2ccc3c4ccc(-c5ccccc5)cc4n(-c4nc(-c5ccccc5)nc(-c5ccccc5)n4)c3c21] 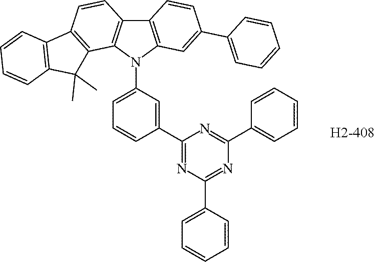 [SMILES: CC1(C)c2ccccc2-c2ccc3c4ccc(-c5ccccc5)cc4n(-c4cccc(-c5nc(-c6ccccc6)nc(-c6ccccc6)n5)c4)c3c21] 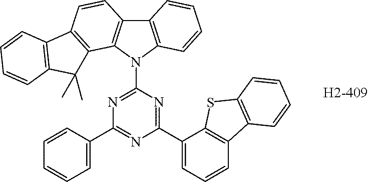 [SMILES: CC1(C)c2ccccc2-c2ccc3c4ccccc4n(-c4nc(-c5ccccc5)nc(-c5cccc6c5sc5ccccc56)n4)c3c21] 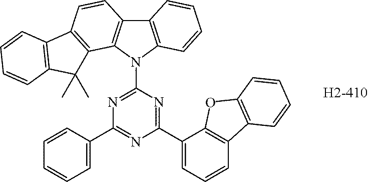 [SMILES: CC1(C)c2ccccc2-c2ccc3c4ccccc4n(-c4nc(-c5ccccc5)nc(-c5cccc6c5oc5ccccc56)n4)c3c21] 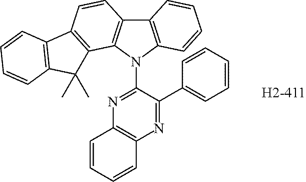 [SMILES: CC1(C)c2ccccc2-c2ccc3c4ccccc4n(-c4nc5ccccc5nc4-c4ccccc4)c3c21] 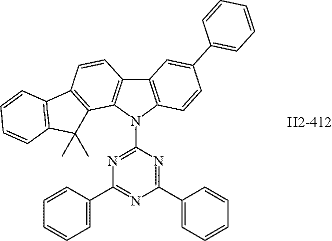 [SMILES: CC1(C)c2ccccc2-c2ccc3c4cc(-c5ccccc5)ccc4n(-c4nc(-c5ccccc5)nc(-c5ccccc5)n4)c3c21] 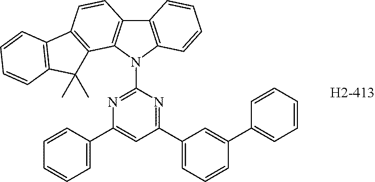 [SMILES: CC1(C)c2ccccc2-c2ccc3c4ccccc4n(-c4nc(-c5ccccc5)cc(-c5cccc(-c6ccccc6)c5)n4)c3c21] 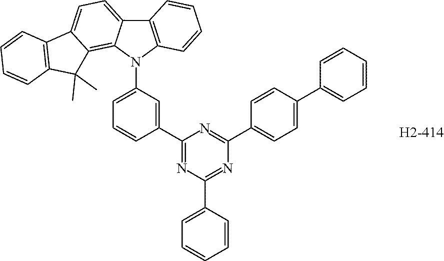 [SMILES: CC1(C)c2ccccc2-c2ccc3c4ccccc4n(-c4cccc(-c5nc(-c6ccccc6)nc(-c6ccc(-c7ccccc7)cc6)n5)c4)c3c21] 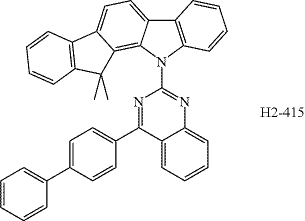 [SMILES: CC1(C)c2ccccc2-c2ccc3c4ccccc4n(-c4nc(-c5ccc(-c6ccccc6)cc5)c5ccccc5n4)c3c21] 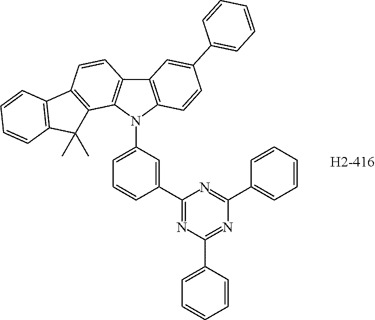 [SMILES: CC1(C)c2ccccc2-c2ccc3c4cc(-c5ccccc5)ccc4n(-c4cccc(-c5nc(-c6ccccc6)nc(-c6ccccc6)n5)c4)c3c21] 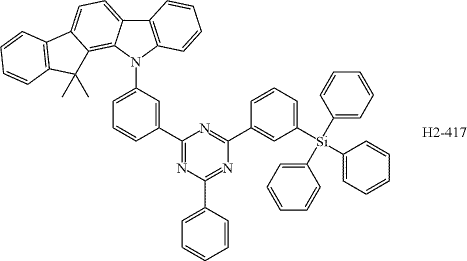 [SMILES: CC1(C)c2ccccc2-c2ccc3c4ccccc4n(-c4cccc(-c5nc(-c6ccccc6)nc(-c6cccc([Si](c7ccccc7)(c7ccccc7)c7ccccc7)c6)n5)c4)c3c21] 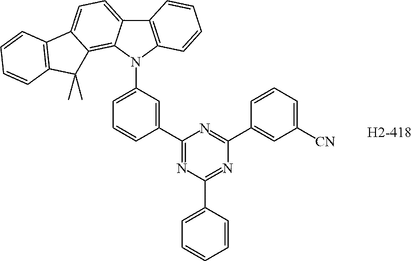 [SMILES: CC1(C)c2ccccc2-c2ccc3c4ccccc4n(-c4cccc(-c5nc(-c6ccccc6)nc(-c6cccc(C#N)c6)n5)c4)c3c21] 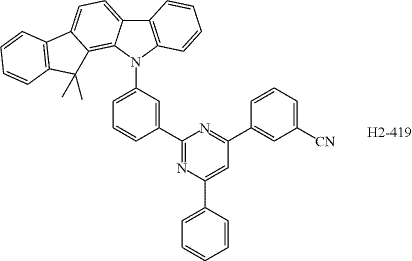 [SMILES: CC1(C)c2ccccc2-c2ccc3c4ccccc4n(-c4cccc(-c5nc(-c6ccccc6)cc(-c6cccc(C#N)c6)n5)c4)c3c21] 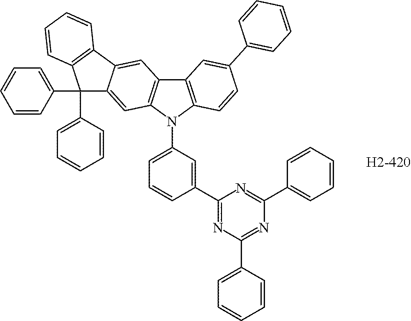 [SMILES: c1ccc(-c2ccc3c(c2)c2cc4c(cc2n3-c2cccc(-c3nc(-c5ccccc5)nc(-c5ccccc5)n3)c2)C(c2ccccc2)(c2ccccc2)c2ccccc2-4)cc1] 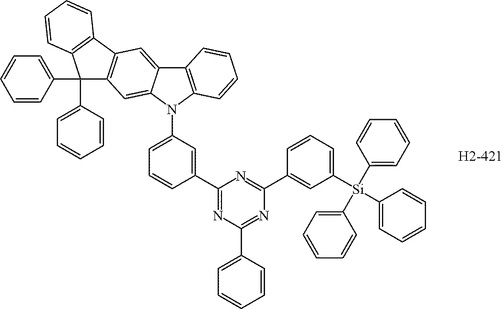 [SMILES: c1ccc(-c2nc(-c3cccc(-n4c5ccccc5c5cc6c(cc54)C(c4ccccc4)(c4ccccc4)c4ccccc4-6)c3)nc(-c3cccc([Si](c4ccccc4)(c4ccccc4)c4ccccc4)c3)n2)cc1] 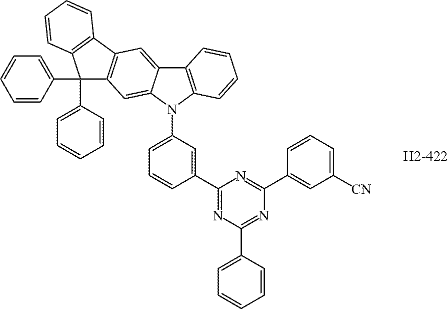 [SMILES: N#Cc1cccc(-c2nc(-c3ccccc3)nc(-c3cccc(-n4c5ccccc5c5cc6c(cc54)C(c4ccccc4)(c4ccccc4)c4ccccc4-6)c3)n2)c1] 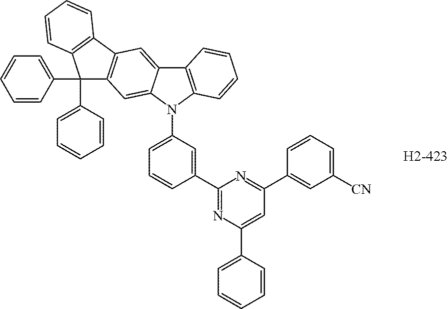 [SMILES: N#Cc1cccc(-c2cc(-c3ccccc3)nc(-c3cccc(-n4c5ccccc5c5cc6c(cc54)C(c4ccccc4)(c4ccccc4)c4ccccc4-6)c3)n2)c1] 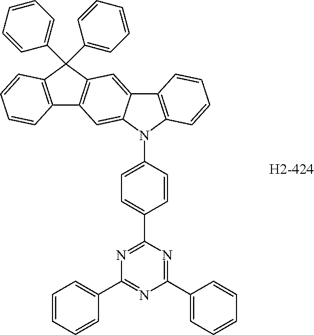 [SMILES: c1ccc(-c2nc(-c3ccccc3)nc(-c3ccc(-n4c5ccccc5c5cc6c(cc54)-c4ccccc4C6(c4ccccc4)c4ccccc4)cc3)n2)cc1] 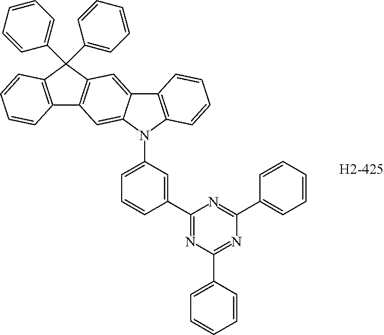 [SMILES: c1ccc(-c2nc(-c3ccccc3)nc(-c3cccc(-n4c5ccccc5c5cc6c(cc54)-c4ccccc4C6(c4ccccc4)c4ccccc4)c3)n2)cc1] 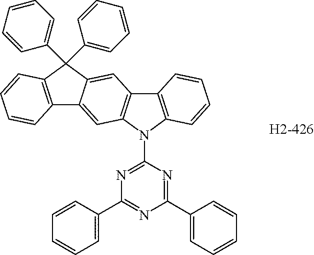 [SMILES: c1ccc(-c2nc(-c3ccccc3)nc(-n3c4ccccc4c4cc5c(cc43)-c3ccccc3C5(c3ccccc3)c3ccccc3)n2)cc1] 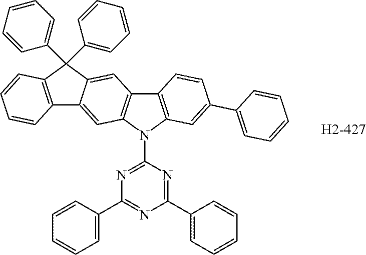 [SMILES: c1ccc(-c2ccc3c4cc5c(cc4n(-c4nc(-c6ccccc6)nc(-c6ccccc6)n4)c3c2)-c2ccccc2C5(c2ccccc2)c2ccccc2)cc1] 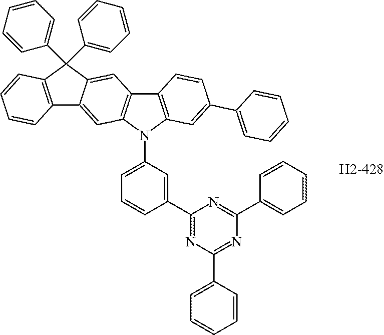 [SMILES: c1ccc(-c2ccc3c4cc5c(cc4n(-c4cccc(-c6nc(-c7ccccc7)nc(-c7ccccc7)n6)c4)c3c2)-c2ccccc2C5(c2ccccc2)c2ccccc2)cc1] 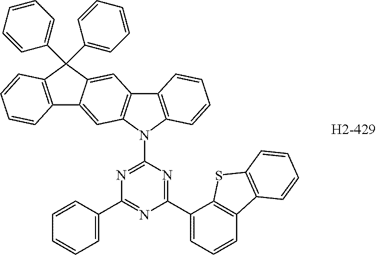 [SMILES: c1ccc(-c2nc(-c3cccc4c3sc3ccccc34)nc(-n3c4ccccc4c4cc5c(cc43)-c3ccccc3C5(c3ccccc3)c3ccccc3)n2)cc1] 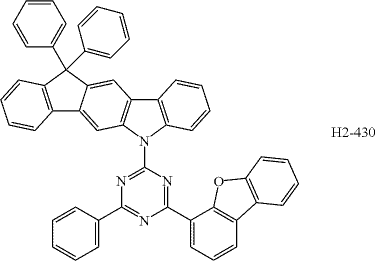 [SMILES: c1ccc(-c2nc(-c3cccc4c3oc3ccccc34)nc(-n3c4ccccc4c4cc5c(cc43)-c3ccccc3C5(c3ccccc3)c3ccccc3)n2)cc1] 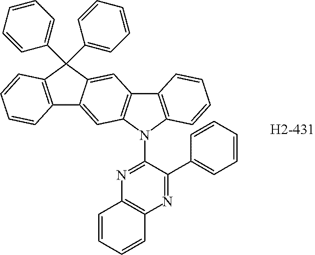 [SMILES: c1ccc(-c2nc3ccccc3nc2-n2c3ccccc3c3cc4c(cc32)-c2ccccc2C4(c2ccccc2)c2ccccc2)cc1] 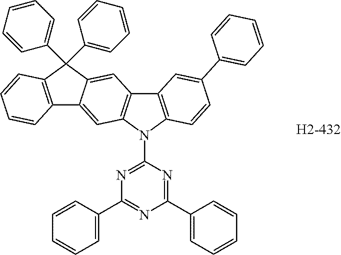 [SMILES: c1ccc(-c2ccc3c(c2)c2cc4c(cc2n3-c2nc(-c3ccccc3)nc(-c3ccccc3)n2)-c2ccccc2C4(c2ccccc2)c2ccccc2)cc1] 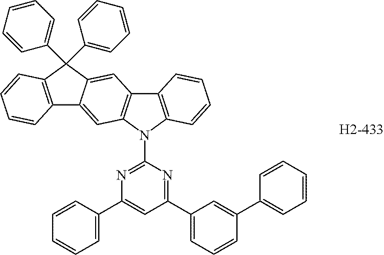 [SMILES: c1ccc(-c2cccc(-c3cc(-c4ccccc4)nc(-n4c5ccccc5c5cc6c(cc54)-c4ccccc4C6(c4ccccc4)c4ccccc4)n3)c2)cc1] 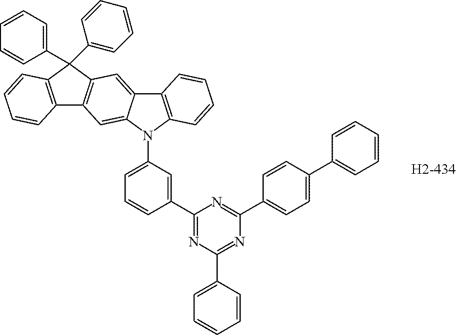 [SMILES: c1ccc(-c2ccc(-c3nc(-c4ccccc4)nc(-c4cccc(-n5c6ccccc6c6cc7c(cc65)-c5ccccc5C7(c5ccccc5)c5ccccc5)c4)n3)cc2)cc1] 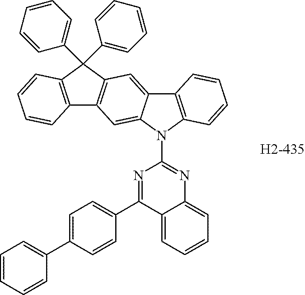 [SMILES: c1ccc(-c2ccc(-c3nc(-n4c5ccccc5c5cc6c(cc54)-c4ccccc4C6(c4ccccc4)c4ccccc4)nc4ccccc34)cc2)cc1] 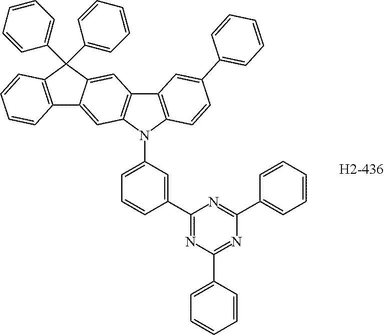 [SMILES: c1ccc(-c2ccc3c(c2)c2cc4c(cc2n3-c2cccc(-c3nc(-c5ccccc5)nc(-c5ccccc5)n3)c2)-c2ccccc2C4(c2ccccc2)c2ccccc2)cc1] 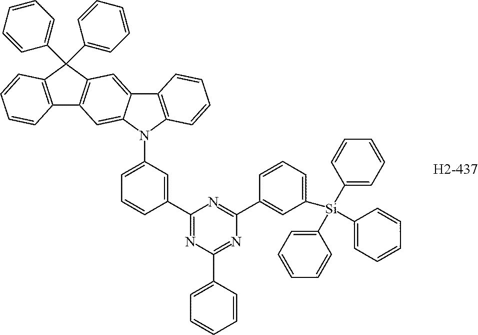 [SMILES: c1ccc(-c2nc(-c3cccc(-n4c5ccccc5c5cc6c(cc54)-c4ccccc4C6(c4ccccc4)c4ccccc4)c3)nc(-c3cccc([Si](c4ccccc4)(c4ccccc4)c4ccccc4)c3)n2)cc1] 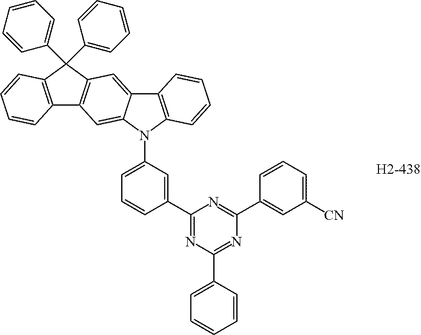 [SMILES: N#Cc1cccc(-c2nc(-c3ccccc3)nc(-c3cccc(-n4c5ccccc5c5cc6c(cc54)-c4ccccc4C6(c4ccccc4)c4ccccc4)c3)n2)c1] 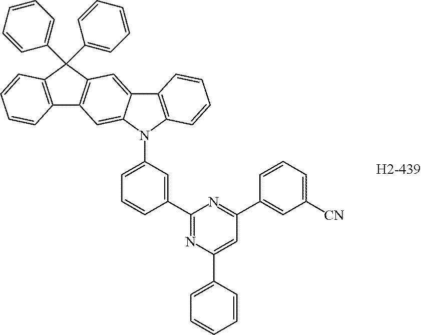 [SMILES: N#Cc1cccc(-c2cc(-c3ccccc3)nc(-c3cccc(-n4c5ccccc5c5cc6c(cc54)-c4ccccc4C6(c4ccccc4)c4ccccc4)c3)n2)c1] 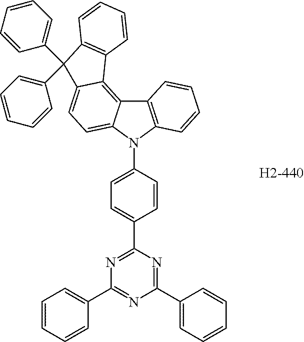 [SMILES: c1ccc(-c2nc(-c3ccccc3)nc(-c3ccc(-n4c5ccccc5c5c6c(ccc54)C(c4ccccc4)(c4ccccc4)c4ccccc4-6)cc3)n2)cc1] 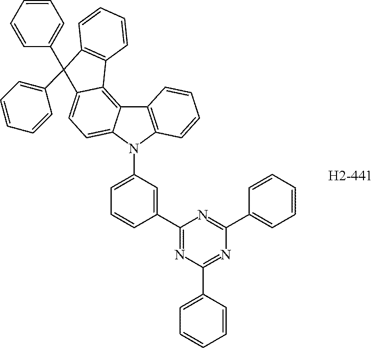 [SMILES: c1ccc(-c2nc(-c3ccccc3)nc(-c3cccc(-n4c5ccccc5c5c6c(ccc54)C(c4ccccc4)(c4ccccc4)c4ccccc4-6)c3)n2)cc1] 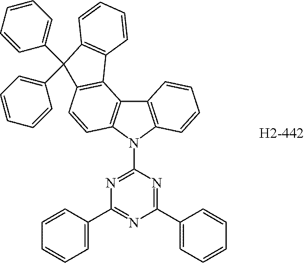 [SMILES: c1ccc(-c2nc(-c3ccccc3)nc(-n3c4ccccc4c4c5c(ccc43)C(c3ccccc3)(c3ccccc3)c3ccccc3-5)n2)cc1] 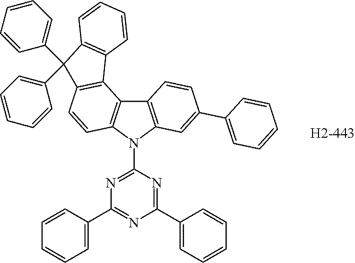 [SMILES: c1ccc(-c2ccc3c4c5c(ccc4n(-c4nc(-c6ccccc6)nc(-c6ccccc6)n4)c3c2)C(c2ccccc2)(c2ccccc2)c2ccccc2-5)cc1] 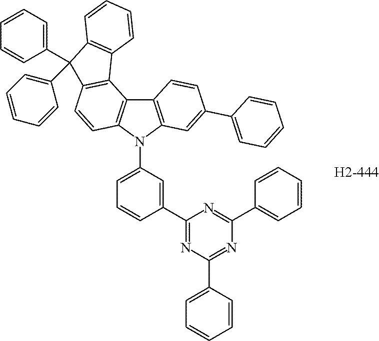 [SMILES: c1ccc(-c2ccc3c4c5c(ccc4n(-c4cccc(-c6nc(-c7ccccc7)nc(-c7ccccc7)n6)c4)c3c2)C(c2ccccc2)(c2ccccc2)c2ccccc2-5)cc1] 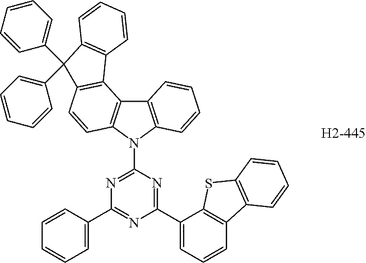 [SMILES: c1ccc(-c2nc(-c3cccc4c3sc3ccccc34)nc(-n3c4ccccc4c4c5c(ccc43)C(c3ccccc3)(c3ccccc3)c3ccccc3-5)n2)cc1] 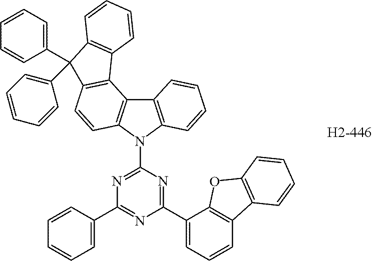 [SMILES: c1ccc(-c2nc(-c3cccc4c3oc3ccccc34)nc(-n3c4ccccc4c4c5c(ccc43)C(c3ccccc3)(c3ccccc3)c3ccccc3-5)n2)cc1] 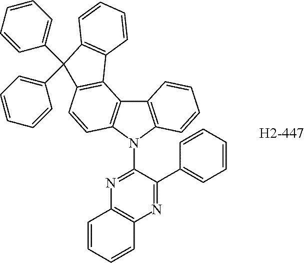 [SMILES: c1ccc(-c2nc3ccccc3nc2-n2c3ccccc3c3c4c(ccc32)C(c2ccccc2)(c2ccccc2)c2ccccc2-4)cc1] 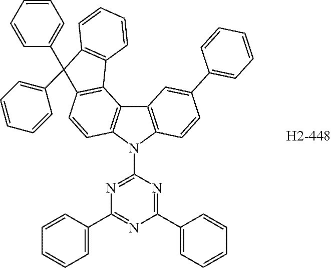 [SMILES: c1ccc(-c2ccc3c(c2)c2c4c(ccc2n3-c2nc(-c3ccccc3)nc(-c3ccccc3)n2)C(c2ccccc2)(c2ccccc2)c2ccccc2-4)cc1] 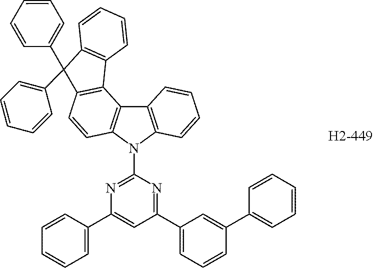 [SMILES: c1ccc(-c2cccc(-c3cc(-c4ccccc4)nc(-n4c5ccccc5c5c6c(ccc54)C(c4ccccc4)(c4ccccc4)c4ccccc4-6)n3)c2)cc1] 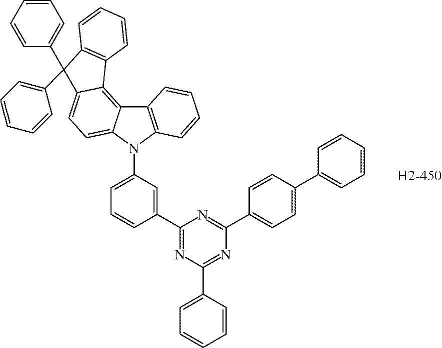 [SMILES: c1ccc(-c2ccc(-c3nc(-c4ccccc4)nc(-c4cccc(-n5c6ccccc6c6c7c(ccc65)C(c5ccccc5)(c5ccccc5)c5ccccc5-7)c4)n3)cc2)cc1] 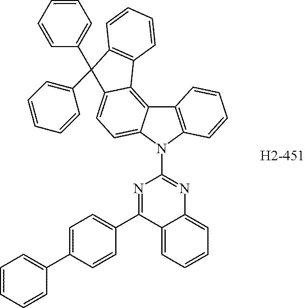 [SMILES: c1ccc(-c2ccc(-c3nc(-n4c5ccccc5c5c6c(ccc54)C(c4ccccc4)(c4ccccc4)c4ccccc4-6)nc4ccccc34)cc2)cc1] 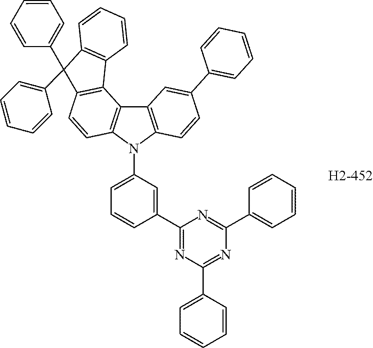 [SMILES: c1ccc(-c2ccc3c(c2)c2c4c(ccc2n3-c2cccc(-c3nc(-c5ccccc5)nc(-c5ccccc5)n3)c2)C(c2ccccc2)(c2ccccc2)c2ccccc2-4)cc1] 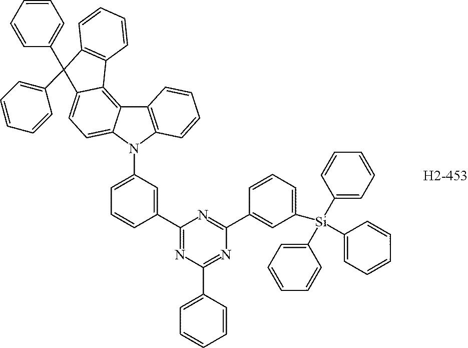 [SMILES: c1ccc(-c2nc(-c3cccc(-n4c5ccccc5c5c6c(ccc54)C(c4ccccc4)(c4ccccc4)c4ccccc4-6)c3)nc(-c3cccc([Si](c4ccccc4)(c4ccccc4)c4ccccc4)c3)n2)cc1] 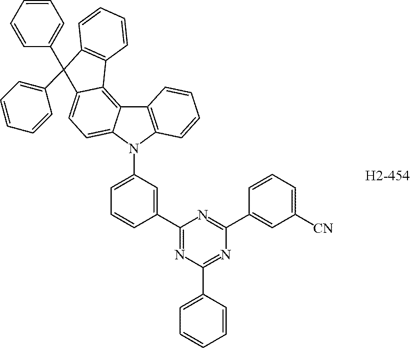 [SMILES: N#Cc1cccc(-c2nc(-c3ccccc3)nc(-c3cccc(-n4c5ccccc5c5c6c(ccc54)C(c4ccccc4)(c4ccccc4)c4ccccc4-6)c3)n2)c1] 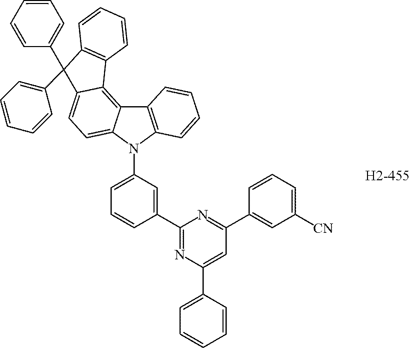 [SMILES: N#Cc1cccc(-c2cc(-c3ccccc3)nc(-c3cccc(-n4c5ccccc5c5c6c(ccc54)C(c4ccccc4)(c4ccccc4)c4ccccc4-6)c3)n2)c1] 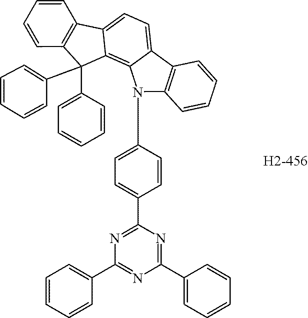 [SMILES: c1ccc(-c2nc(-c3ccccc3)nc(-c3ccc(-n4c5ccccc5c5ccc6c(c54)C(c4ccccc4)(c4ccccc4)c4ccccc4-6)cc3)n2)cc1] 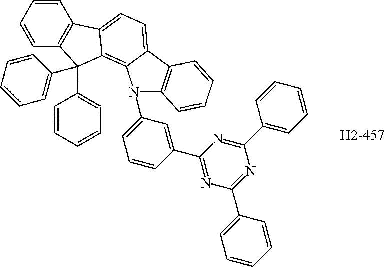 [SMILES: c1ccc(-c2nc(-c3ccccc3)nc(-c3cccc(-n4c5ccccc5c5ccc6c(c54)C(c4ccccc4)(c4ccccc4)c4ccccc4-6)c3)n2)cc1] 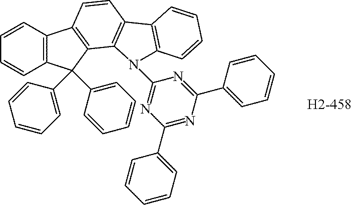 [SMILES: c1ccc(-c2nc(-c3ccccc3)nc(-n3c4ccccc4c4ccc5c(c43)C(c3ccccc3)(c3ccccc3)c3ccccc3-5)n2)cc1] 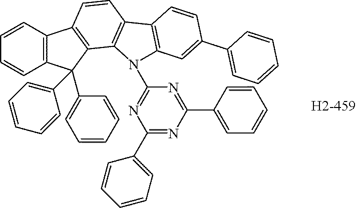 [SMILES: c1ccc(-c2ccc3c4ccc5c(c4n(-c4nc(-c6ccccc6)nc(-c6ccccc6)n4)c3c2)C(c2ccccc2)(c2ccccc2)c2ccccc2-5)cc1] 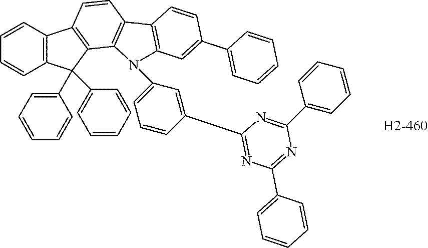 [SMILES: c1ccc(-c2ccc3c4ccc5c(c4n(-c4cccc(-c6nc(-c7ccccc7)nc(-c7ccccc7)n6)c4)c3c2)C(c2ccccc2)(c2ccccc2)c2ccccc2-5)cc1] 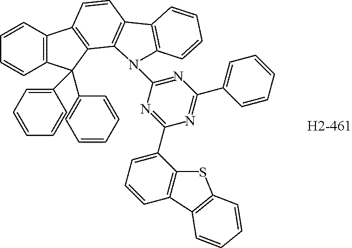 [SMILES: c1ccc(-c2nc(-c3cccc4c3sc3ccccc34)nc(-n3c4ccccc4c4ccc5c(c43)C(c3ccccc3)(c3ccccc3)c3ccccc3-5)n2)cc1] 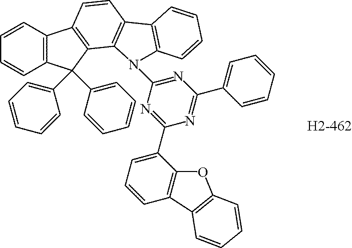 [SMILES: c1ccc(-c2nc(-c3cccc4c3oc3ccccc34)nc(-n3c4ccccc4c4ccc5c(c43)C(c3ccccc3)(c3ccccc3)c3ccccc3-5)n2)cc1] 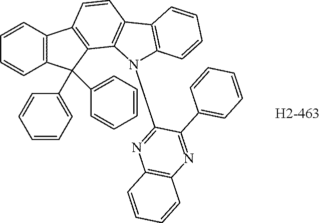 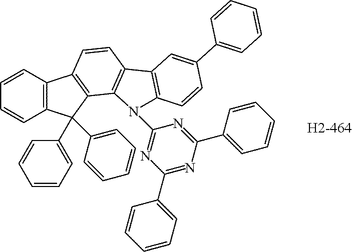 [SMILES: c1ccc(-c2ccc3c(c2)c2ccc4c(c2n3-c2nc(-c3ccccc3)nc(-c3ccccc3)n2)C(c2ccccc2)(c2ccccc2)c2ccccc2-4)cc1] 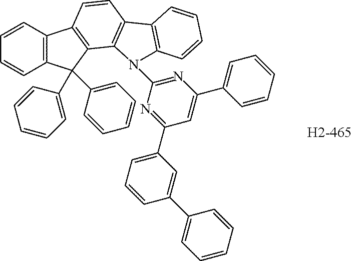 [SMILES: c1ccc(-c2cccc(-c3cc(-c4ccccc4)nc(-n4c5ccccc5c5ccc6c(c54)C(c4ccccc4)(c4ccccc4)c4ccccc4-6)n3)c2)cc1] 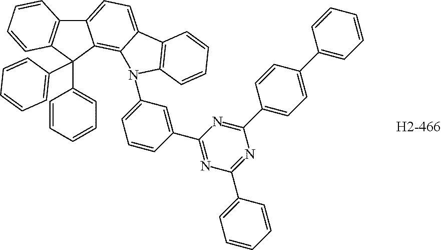 [SMILES: c1ccc(-c2ccc(-c3nc(-c4ccccc4)nc(-c4cccc(-n5c6ccccc6c6ccc7c(c65)C(c5ccccc5)(c5ccccc5)c5ccccc5-7)c4)n3)cc2)cc1] 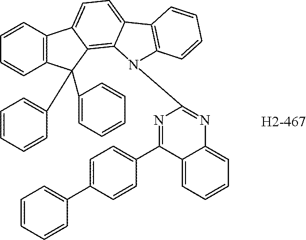 [SMILES: c1ccc(-c2ccc(-c3nc(-n4c5ccccc5c5ccc6c(c54)C(c4ccccc4)(c4ccccc4)c4ccccc4-6)nc4ccccc34)cc2)cc1] 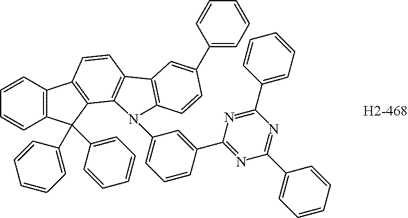 [SMILES: c1ccc(-c2ccc3c(c2)c2ccc4c(c2n3-c2cccc(-c3nc(-c5ccccc5)nc(-c5ccccc5)n3)c2)C(c2ccccc2)(c2ccccc2)c2ccccc2-4)cc1] 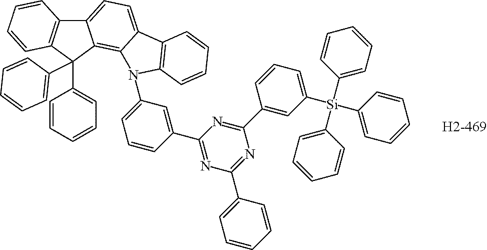 [SMILES: c1ccc(-c2nc(-c3cccc(-n4c5ccccc5c5ccc6c(c54)C(c4ccccc4)(c4ccccc4)c4ccccc4-6)c3)nc(-c3cccc([Si](c4ccccc4)(c4ccccc4)c4ccccc4)c3)n2)cc1] 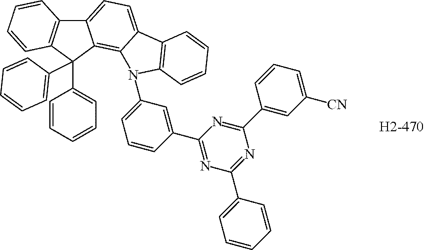 [SMILES: N#Cc1cccc(-c2nc(-c3ccccc3)nc(-c3cccc(-n4c5ccccc5c5ccc6c(c54)C(c4ccccc4)(c4ccccc4)c4ccccc4-6)c3)n2)c1] 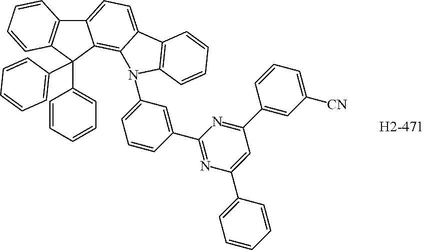 [SMILES: N#Cc1cccc(-c2cc(-c3ccccc3)nc(-c3cccc(-n4c5ccccc5c5ccc6c(c54)C(c4ccccc4)(c4ccccc4)c4ccccc4-6)c3)n2)c1] 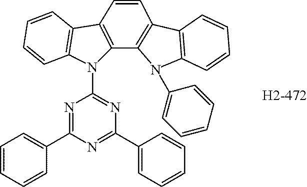 [SMILES: c1ccc(-c2nc(-c3ccccc3)nc(-n3c4ccccc4c4ccc5c6ccccc6n(-c6ccccc6)c5c43)n2)cc1] 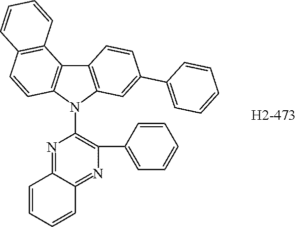 [SMILES: c1ccc(-c2ccc3c4c5ccccc5ccc4n(-c4nc5ccccc5nc4-c4ccccc4)c3c2)cc1] 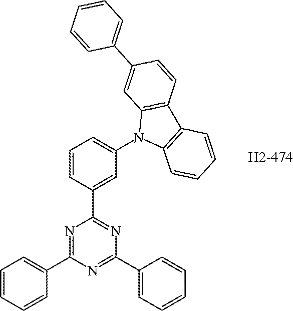 [SMILES: c1ccc(-c2ccc3c4ccccc4n(-c4cccc(-c5nc(-c6ccccc6)nc(-c6ccccc6)n5)c4)c3c2)cc1] 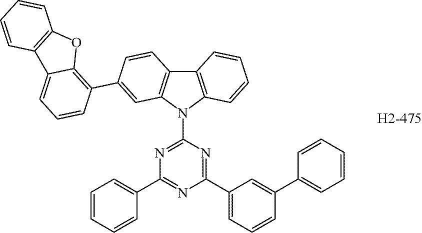 [SMILES: c1ccc(-c2cccc(-c3nc(-c4ccccc4)nc(-n4c5ccccc5c5ccc(-c6cccc7c6oc6ccccc67)cc54)n3)c2)cc1] 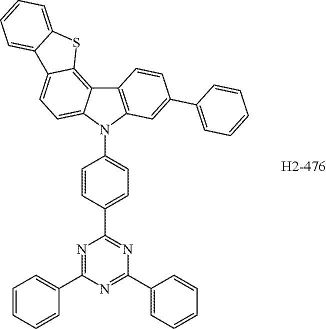 [SMILES: c1ccc(-c2ccc3c4c5sc6ccccc6c5ccc4n(-c4ccc(-c5nc(-c6ccccc6)nc(-c6ccccc6)n5)cc4)c3c2)cc1] 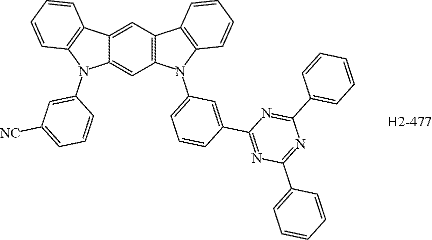 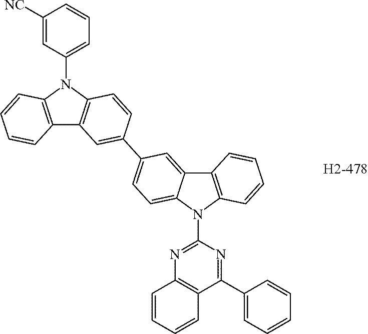 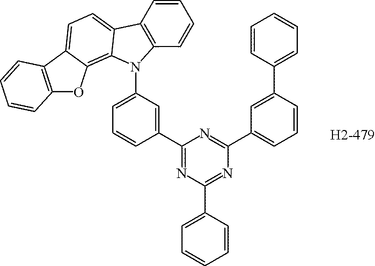 [SMILES: c1ccc(-c2cccc(-c3nc(-c4ccccc4)nc(-c4cccc(-n5c6ccccc6c6ccc7c8ccccc8oc7c65)c4)n3)c2)cc1] 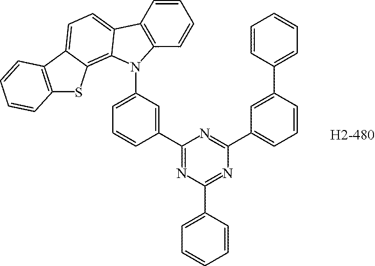 [SMILES: c1ccc(-c2cccc(-c3nc(-c4ccccc4)nc(-c4cccc(-n5c6ccccc6c6ccc7c8ccccc8sc7c65)c4)n3)c2)cc1] 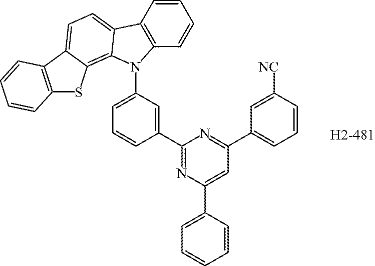 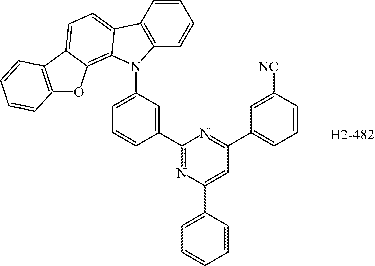 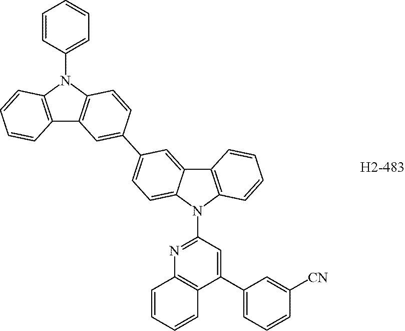 [SMILES: N#Cc1cccc(-c2cc(-n3c4ccccc4c4cc(-c5ccc6c(c5)c5ccccc5n6-c5ccccc5)ccc43)nc3ccccc23)c1] 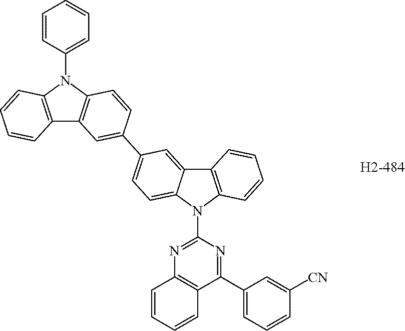 [SMILES: N#Cc1cccc(-c2nc(-n3c4ccccc4c4cc(-c5ccc6c(c5)c5ccccc5n6-c5ccccc5)ccc43)nc3ccccc23)c1] 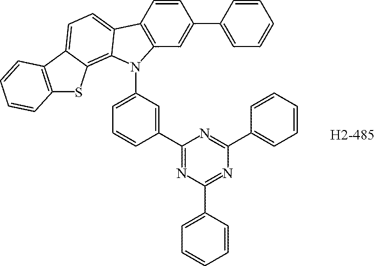 [SMILES: c1ccc(-c2ccc3c4ccc5c6ccccc6sc5c4n(-c4cccc(-c5nc(-c6ccccc6)nc(-c6ccccc6)n5)c4)c3c2)cc1] 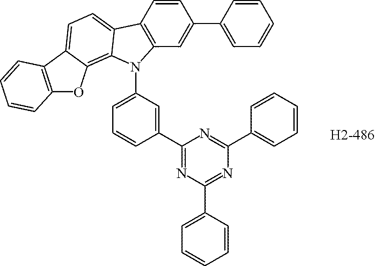 [SMILES: c1ccc(-c2ccc3c4ccc5c6ccccc6oc5c4n(-c4cccc(-c5nc(-c6ccccc6)nc(-c6ccccc6)n5)c4)c3c2)cc1] 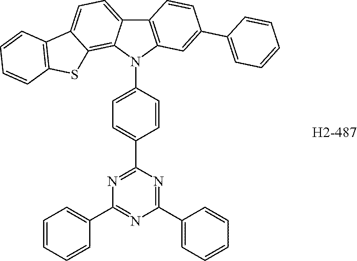 [SMILES: c1ccc(-c2ccc3c4ccc5c6ccccc6sc5c4n(-c4ccc(-c5nc(-c6ccccc6)nc(-c6ccccc6)n5)cc4)c3c2)cc1] 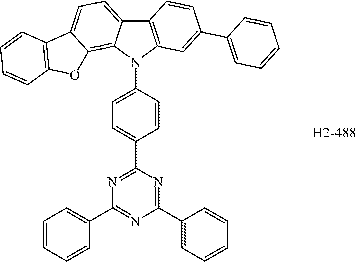 [SMILES: c1ccc(-c2ccc3c4ccc5c6ccccc6oc5c4n(-c4ccc(-c5nc(-c6ccccc6)nc(-c6ccccc6)n5)cc4)c3c2)cc1] 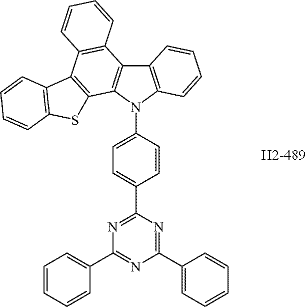 [SMILES: c1ccc(-c2nc(-c3ccccc3)nc(-c3ccc(-n4c5ccccc5c5c6ccccc6c6c7ccccc7sc6c54)cc3)n2)cc1] 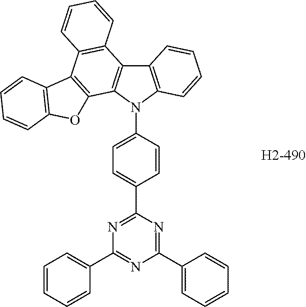 [SMILES: c1ccc(-c2nc(-c3ccccc3)nc(-c3ccc(-n4c5ccccc5c5c6ccccc6c6c7ccccc7oc6c54)cc3)n2)cc1] 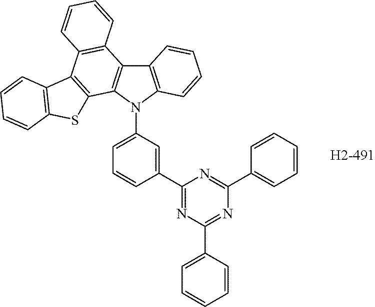 [SMILES: c1ccc(-c2nc(-c3ccccc3)nc(-c3cccc(-n4c5ccccc5c5c6ccccc6c6c7ccccc7sc6c54)c3)n2)cc1] 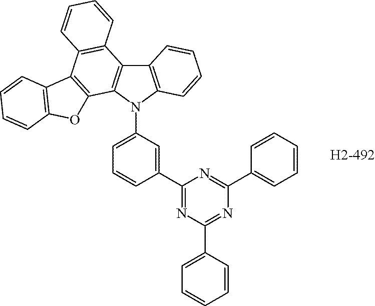 [SMILES: c1ccc(-c2nc(-c3ccccc3)nc(-c3cccc(-n4c5ccccc5c5c6ccccc6c6c7ccccc7oc6c54)c3)n2)cc1] 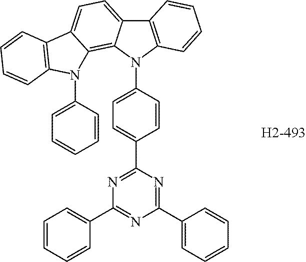 [SMILES: c1ccc(-c2nc(-c3ccccc3)nc(-c3ccc(-n4c5ccccc5c5ccc6c7ccccc7n(-c7ccccc7)c6c54)cc3)n2)cc1] 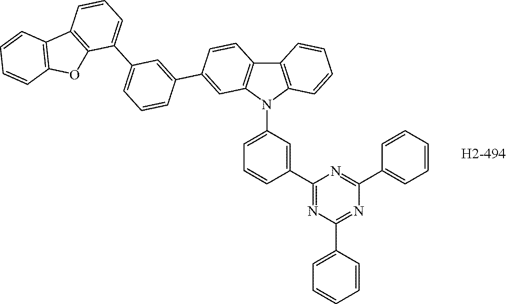 [SMILES: c1ccc(-c2nc(-c3ccccc3)nc(-c3cccc(-n4c5ccccc5c5ccc(-c6cccc(-c7cccc8c7oc7ccccc78)c6)cc54)c3)n2)cc1] 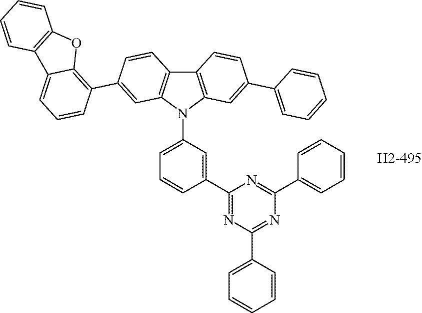 [SMILES: c1ccc(-c2ccc3c4ccc(-c5cccc6c5oc5ccccc56)cc4n(-c4cccc(-c5nc(-c6ccccc6)nc(-c6ccccc6)n5)c4)c3c2)cc1] 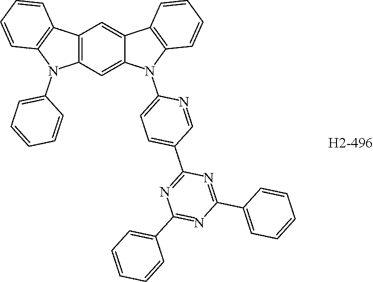 [SMILES: c1ccc(-c2nc(-c3ccccc3)nc(-c3ccc(-n4c5ccccc5c5cc6c7ccccc7n(-c7ccccc7)c6cc54)nc3)n2)cc1] 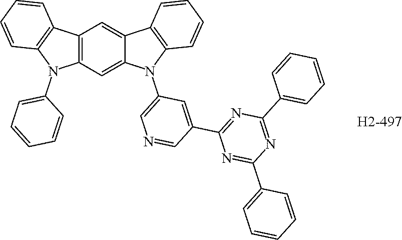 [SMILES: c1ccc(-c2nc(-c3ccccc3)nc(-c3cncc(-n4c5ccccc5c5cc6c7ccccc7n(-c7ccccc7)c6cc54)c3)n2)cc1] 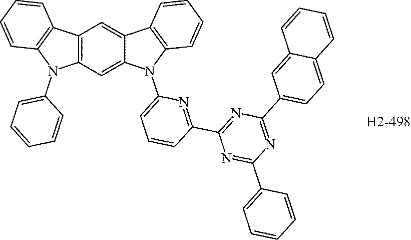 [SMILES: c1ccc(-c2nc(-c3ccc4ccccc4c3)nc(-c3cccc(-n4c5ccccc5c5cc6c7ccccc7n(-c7ccccc7)c6cc54)n3)n2)cc1] 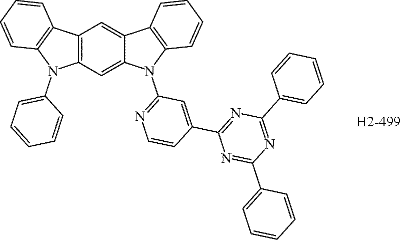 [SMILES: c1ccc(-c2nc(-c3ccccc3)nc(-c3ccnc(-n4c5ccccc5c5cc6c7ccccc7n(-c7ccccc7)c6cc54)c3)n2)cc1] 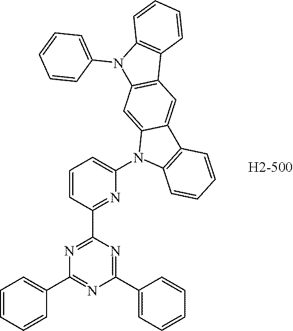 [SMILES: c1ccc(-c2nc(-c3ccccc3)nc(-c3cccc(-n4c5ccccc5c5cc6c7ccccc7n(-c7ccccc7)c6cc54)n3)n2)cc1] 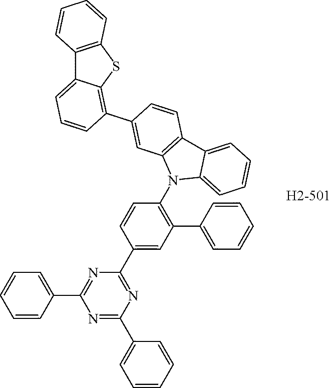 [SMILES: c1ccc(-c2nc(-c3ccccc3)nc(-c3ccc(-n4c5ccccc5c5ccc(-c6cccc7c6sc6ccccc67)cc54)c(-c4ccccc4)c3)n2)cc1] 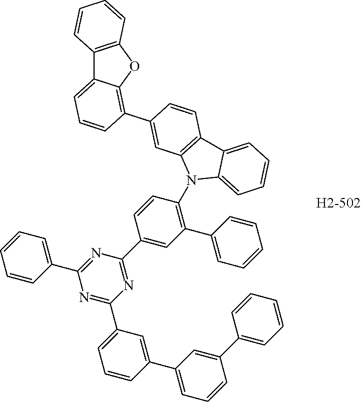 [SMILES: c1ccc(-c2cccc(-c3cccc(-c4nc(-c5ccccc5)nc(-c5ccc(-n6c7ccccc7c7ccc(-c8cccc9c8oc8ccccc89)cc76)c(-c6ccccc6)c5)n4)c3)c2)cc1] 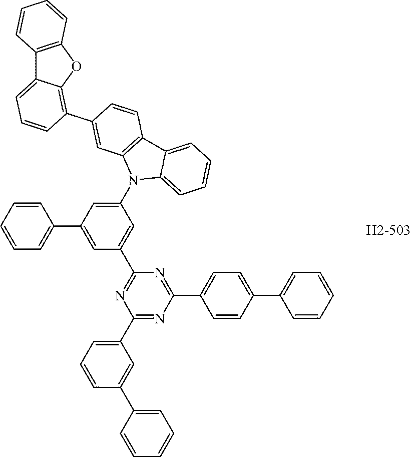 [SMILES: c1ccc(-c2ccc(-c3nc(-c4cccc(-c5ccccc5)c4)nc(-c4cc(-c5ccccc5)cc(-n5c6ccccc6c6ccc(-c7cccc8c7oc7ccccc78)cc65)c4)n3)cc2)cc1] 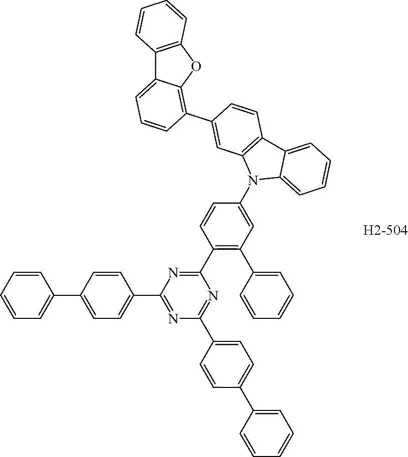 [SMILES: c1ccc(-c2ccc(-c3nc(-c4ccc(-c5ccccc5)cc4)nc(-c4ccc(-n5c6ccccc6c6ccc(-c7cccc8c7oc7ccccc78)cc65)cc4-c4ccccc4)n3)cc2)cc1] 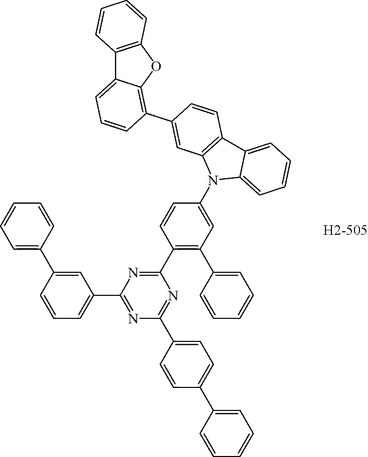 [SMILES: c1ccc(-c2ccc(-c3nc(-c4cccc(-c5ccccc5)c4)nc(-c4ccc(-n5c6ccccc6c6ccc(-c7cccc8c7oc7ccccc78)cc65)cc4-c4ccccc4)n3)cc2)cc1] 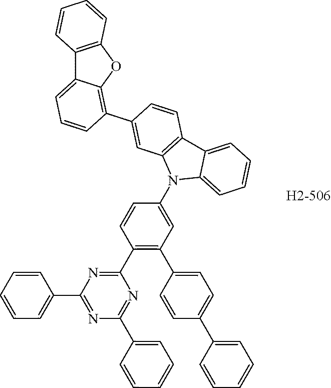 [SMILES: c1ccc(-c2ccc(-c3cc(-n4c5ccccc5c5ccc(-c6cccc7c6oc6ccccc67)cc54)ccc3-c3nc(-c4ccccc4)nc(-c4ccccc4)n3)cc2)cc1] 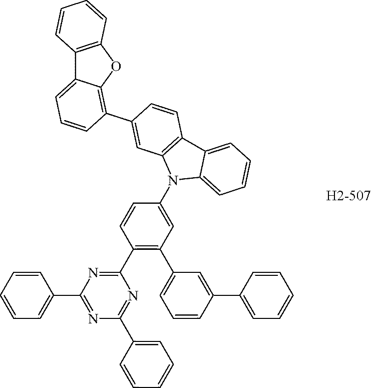 [SMILES: c1ccc(-c2cccc(-c3cc(-n4c5ccccc5c5ccc(-c6cccc7c6oc6ccccc67)cc54)ccc3-c3nc(-c4ccccc4)nc(-c4ccccc4)n3)c2)cc1] 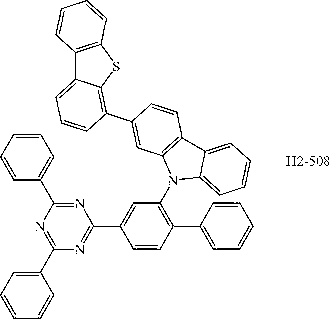 [SMILES: c1ccc(-c2nc(-c3ccccc3)nc(-c3ccc(-c4ccccc4)c(-n4c5ccccc5c5ccc(-c6cccc7c6sc6ccccc67)cc54)c3)n2)cc1] 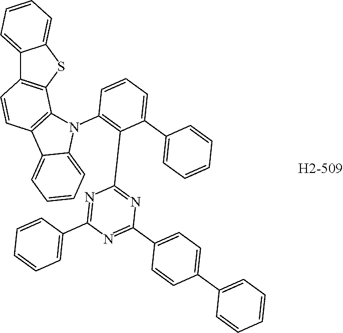 [SMILES: c1ccc(-c2ccc(-c3nc(-c4ccccc4)nc(-c4c(-c5ccccc5)cccc4-n4c5ccccc5c5ccc6c7ccccc7sc6c54)n3)cc2)cc1] 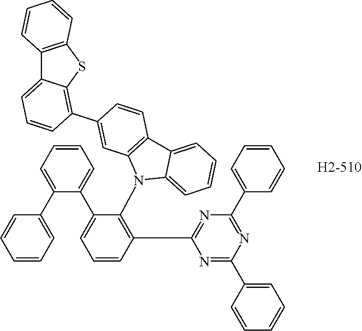 [SMILES: c1ccc(-c2nc(-c3ccccc3)nc(-c3cccc(-c4ccccc4-c4ccccc4)c3-n3c4ccccc4c4ccc(-c5cccc6c5sc5ccccc56)cc43)n2)cc1] 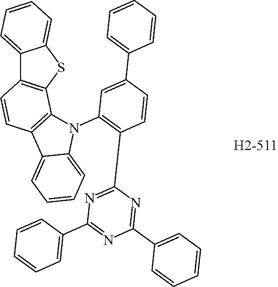 [SMILES: c1ccc(-c2ccc(-c3nc(-c4ccccc4)nc(-c4ccccc4)n3)c(-n3c4ccccc4c4ccc5c6ccccc6sc5c43)c2)cc1] 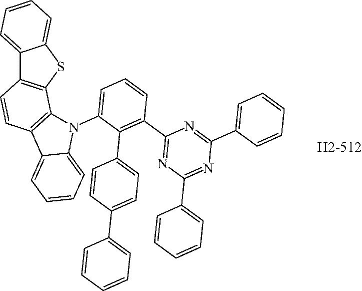 [SMILES: c1ccc(-c2ccc(-c3c(-c4nc(-c5ccccc5)nc(-c5ccccc5)n4)cccc3-n3c4ccccc4c4ccc5c6ccccc6sc5c43)cc2)cc1] 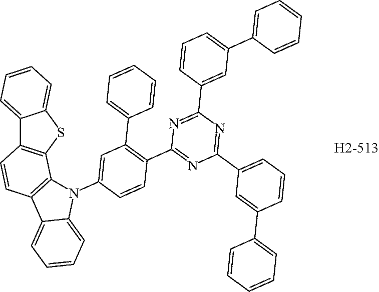 [SMILES: c1ccc(-c2cccc(-c3nc(-c4cccc(-c5ccccc5)c4)nc(-c4ccc(-n5c6ccccc6c6ccc7c8ccccc8sc7c65)cc4-c4ccccc4)n3)c2)cc1] 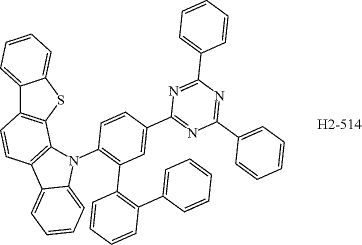 [SMILES: c1ccc(-c2nc(-c3ccccc3)nc(-c3ccc(-n4c5ccccc5c5ccc6c7ccccc7sc6c54)c(-c4ccccc4-c4ccccc4)c3)n2)cc1] 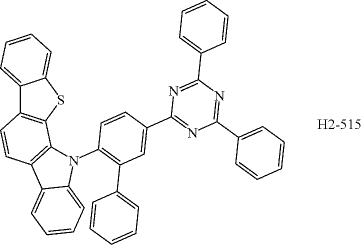 [SMILES: c1ccc(-c2nc(-c3ccccc3)nc(-c3ccc(-n4c5ccccc5c5ccc6c7ccccc7sc6c54)c(-c4ccccc4)c3)n2)cc1] 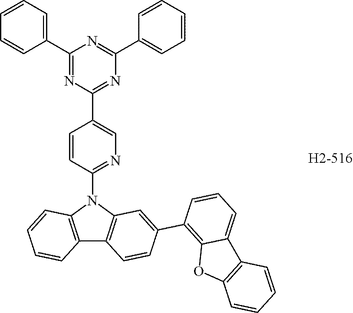 [SMILES: c1ccc(-c2nc(-c3ccccc3)nc(-c3ccc(-n4c5ccccc5c5ccc(-c6cccc7c6oc6ccccc67)cc54)nc3)n2)cc1] 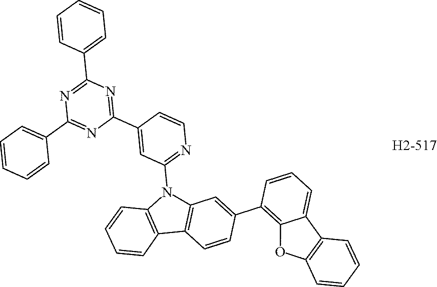 [SMILES: c1ccc(-c2nc(-c3ccccc3)nc(-c3ccnc(-n4c5ccccc5c5ccc(-c6cccc7c6oc6ccccc67)cc54)c3)n2)cc1] 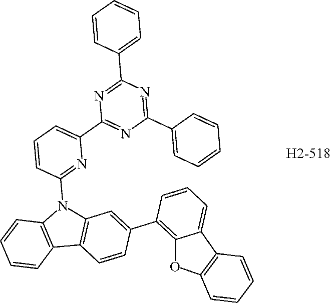 [SMILES: c1ccc(-c2nc(-c3ccccc3)nc(-c3cccc(-n4c5ccccc5c5ccc(-c6cccc7c6oc6ccccc67)cc54)n3)n2)cc1] 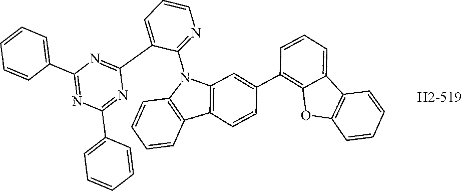 [SMILES: c1ccc(-c2nc(-c3ccccc3)nc(-c3cccnc3-n3c4ccccc4c4ccc(-c5cccc6c5oc5ccccc56)cc43)n2)cc1]
and 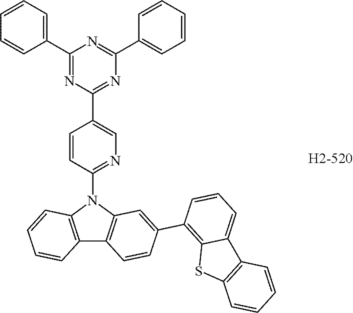 [SMILES: c1ccc(-c2nc(-c3ccccc3)nc(-c3ccc(-n4c5ccccc5c5ccc(-c6cccc7c6sc6ccccc67)cc54)nc3)n2)cc1]
.

7. An organic electroluminescent compound represented by the following formula 5: 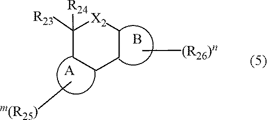
in formula 5,
X₂ represents —N═, —NR₀—, —O— or —S—;
R₀, R₂₃ and R₂₄, each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di- (3- to 30-membered)heteroarylamino, or a substituted or unsubstituted (C6-C30)aryl(3- to 30-membered)heteroarylamino;
A and B, each independently, represent a mono- or polycyclic, (C5-C20) alicyclic or aromatic ring, or the combination thereof, in which a carbon atom(s) of the ring may be replaced with at least one heteroatom selected from N, O, and S;
R₂₅ and R₂₆, each independently, represent hydrogen, deuterium, a substituted or unsubstituted (C1-C30)alkyl, a substituted or unsubstituted (C2-C30)alkenyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted fused ring group of a (C3-C30) aliphatic ring(s) and a (C6-C30) aromatic ring(s), a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di- (C6-C30)arylamino, a substituted or unsubstituted mono- or di- (3- to 30-membered)heteroarylamino, a substituted or unsubstituted (C6-C30)aryl(3- to 30-membered)heteroarylamino, or —(L₁)ₐ—Ar₁; with the proviso that at least one of R₂₅ and R₂₆ represents —(L₁)ₐ—Ar₁;
L₁, each independently, represents a single bond, a substituted or unsubstituted (C6-C30)arylene, or a substituted or unsubstituted (3- to 30-membered)heteroarylene;
Ar₁ represents a substituted or unsubstituted (C6-C30)aryl, a substituted or unsubstituted (3- to 30-membered)heteroaryl, or 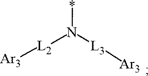
L₂ and L₃, each independently, represent a single bond, a substituted or unsubstituted (C6-C30)arylene, or a substituted or unsubstituted (3- to 30-membered)heteroarylene;
Ar₂ and Ar₃, each independently, represent a substituted or unsubstituted (C6-C30)aryl, a substituted or unsubstituted (3- to 30-membered)heteroaryl, or a substituted or unsubstituted (C3-C30)cycloalkyl;
m and n, each independently, represent an integer of 1 to 4, where if m and n are an integer of 2 or more, each of R₂₅ and each of R₂₆ may be the same or different; and a represents an integer of 1 or 2, where if a is an integer of 2, each of L₁ may be the same or different.

8. The organic electroluminescent compound according to claim 7, wherein formula 5 is represented by any one of the following formulas 12 to 18: 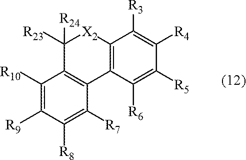 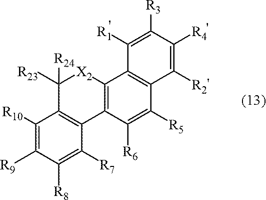 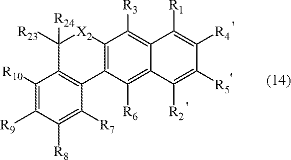 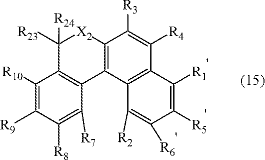 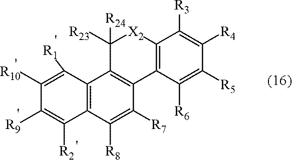 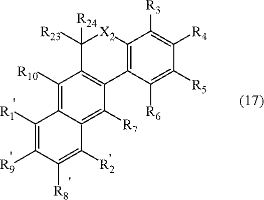 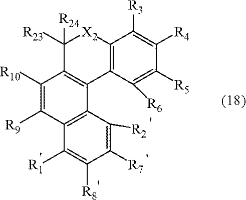
in formulas 12 to 18,
R₃ to R₁₀, and R₁′ to R₁₀′, each independently, are as defined for R₂₅ and R₂₆ in claim 7, with the proviso that at least one of R₃ to R₁₀, and R₁′ to R₁₀′ represents —(L₁)ₐ—Ar₁; and
X₂, R₂₃, R₂₄, L₁, Ar₁, and a are as defined in claim 7.

9. The organic electroluminescent compound according to claim 7, wherein the compound represented by formula 5 is selected from the group consisting of the following compounds: 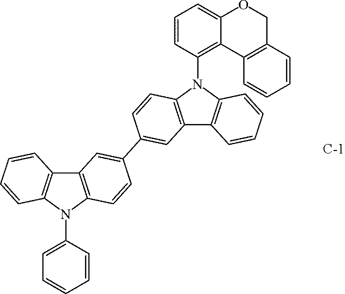 [SMILES: c1ccc(-n2c3ccccc3c3cc(-c4ccc5c(c4)c4ccccc4n5-c4cccc5c4-c4ccccc4CO5)ccc32)cc1] 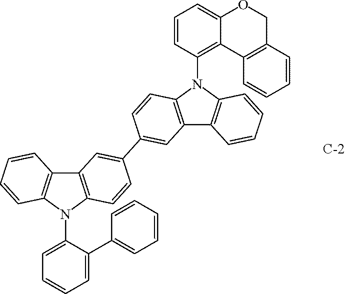 [SMILES: c1ccc(-c2ccccc2-n2c3ccccc3c3cc(-c4ccc5c(c4)c4ccccc4n5-c4cccc5c4-c4ccccc4CO5)ccc32)cc1] 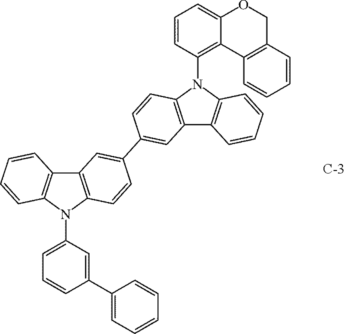 [SMILES: c1ccc(-c2cccc(-n3c4ccccc4c4cc(-c5ccc6c(c5)c5ccccc5n6-c5cccc6c5-c5ccccc5CO6)ccc43)c2)cc1] 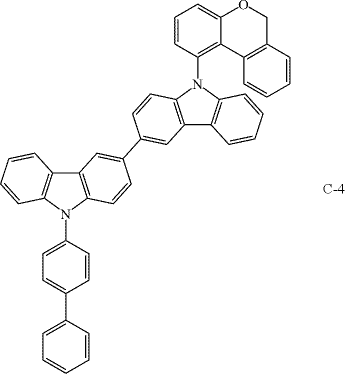 [SMILES: c1ccc(-c2ccc(-n3c4ccccc4c4cc(-c5ccc6c(c5)c5ccccc5n6-c5cccc6c5-c5ccccc5CO6)ccc43)cc2)cc1] 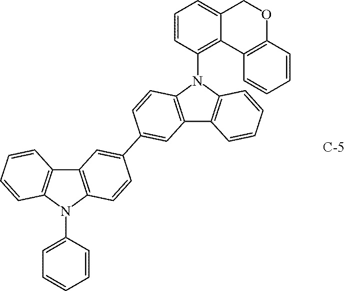 [SMILES: c1ccc(-n2c3ccccc3c3cc(-c4ccc5c(c4)c4ccccc4n5-c4cccc5c4-c4ccccc4OC5)ccc32)cc1] 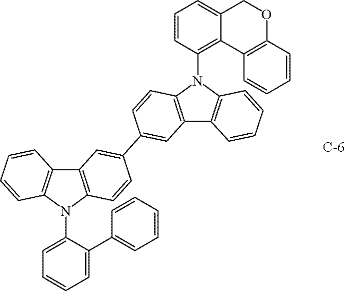 [SMILES: c1ccc(-c2ccccc2-n2c3ccccc3c3cc(-c4ccc5c(c4)c4ccccc4n5-c4cccc5c4-c4ccccc4OC5)ccc32)cc1] 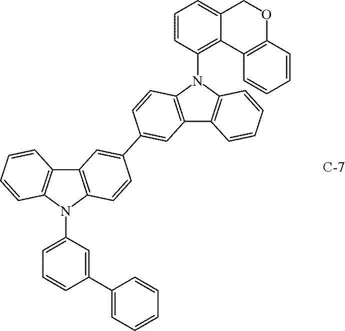 [SMILES: c1ccc(-c2cccc(-n3c4ccccc4c4cc(-c5ccc6c(c5)c5ccccc5n6-c5cccc6c5-c5ccccc5OC6)ccc43)c2)cc1] 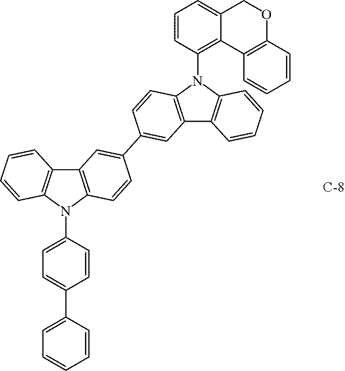 [SMILES: c1ccc(-c2ccc(-n3c4ccccc4c4cc(-c5ccc6c(c5)c5ccccc5n6-c5cccc6c5-c5ccccc5OC6)ccc43)cc2)cc1] 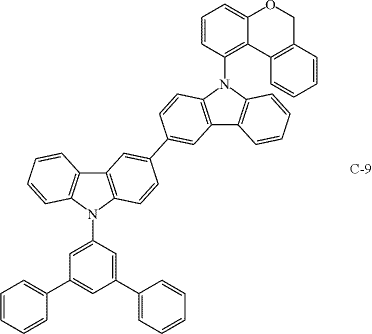 [SMILES: c1ccc(-c2cc(-c3ccccc3)cc(-n3c4ccccc4c4cc(-c5ccc6c(c5)c5ccccc5n6-c5cccc6c5-c5ccccc5CO6)ccc43)c2)cc1] 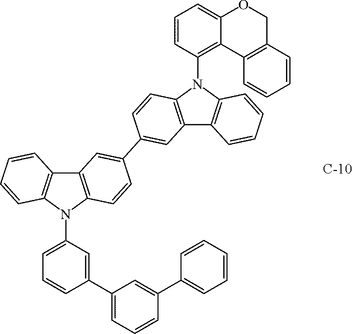 [SMILES: c1ccc(-c2cccc(-c3cccc(-n4c5ccccc5c5cc(-c6ccc7c(c6)c6ccccc6n7-c6cccc7c6-c6ccccc6CO7)ccc54)c3)c2)cc1] 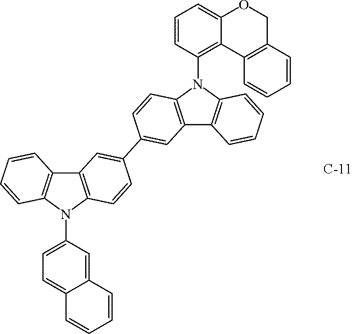 [SMILES: c1ccc2c(c1)COc1cccc(-n3c4ccccc4c4cc(-c5ccc6c(c5)c5ccccc5n6-c5ccc6ccccc6c5)ccc43)c1-2] 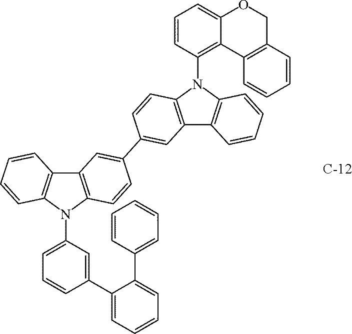 [SMILES: c1ccc(-c2ccccc2-c2cccc(-n3c4ccccc4c4cc(-c5ccc6c(c5)c5ccccc5n6-c5cccc6c5-c5ccccc5CO6)ccc43)c2)cc1] 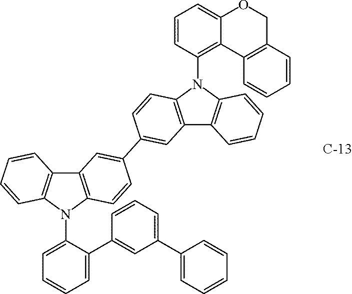 [SMILES: c1ccc(-c2cccc(-c3ccccc3-n3c4ccccc4c4cc(-c5ccc6c(c5)c5ccccc5n6-c5cccc6c5-c5ccccc5CO6)ccc43)c2)cc1] 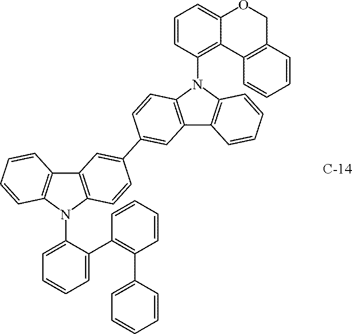 [SMILES: c1ccc(-c2ccccc2-c2ccccc2-n2c3ccccc3c3cc(-c4ccc5c(c4)c4ccccc4n5-c4cccc5c4-c4ccccc4CO5)ccc32)cc1] 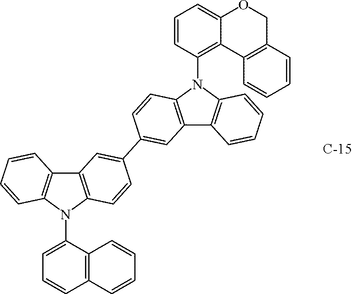 [SMILES: c1ccc2c(c1)COc1cccc(-n3c4ccccc4c4cc(-c5ccc6c(c5)c5ccccc5n6-c5cccc6ccccc56)ccc43)c1-2] 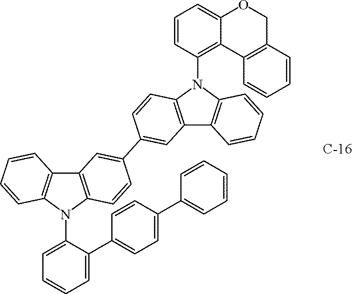 [SMILES: c1ccc(-c2ccc(-c3ccccc3-n3c4ccccc4c4cc(-c5ccc6c(c5)c5ccccc5n6-c5cccc6c5-c5ccccc5CO6)ccc43)cc2)cc1] 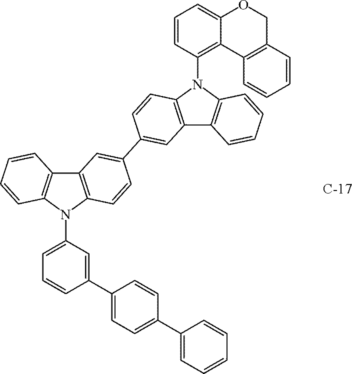 [SMILES: c1ccc(-c2ccc(-c3cccc(-n4c5ccccc5c5cc(-c6ccc7c(c6)c6ccccc6n7-c6cccc7c6-c6ccccc6CO7)ccc54)c3)cc2)cc1] 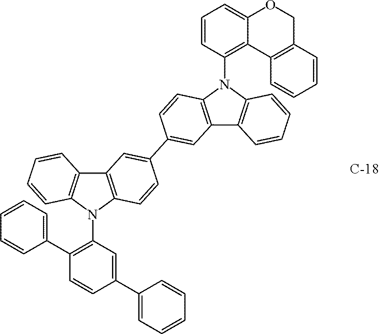 [SMILES: c1ccc(-c2ccc(-c3ccccc3)c(-n3c4ccccc4c4cc(-c5ccc6c(c5)c5ccccc5n6-c5cccc6c5-c5ccccc5CO6)ccc43)c2)cc1] 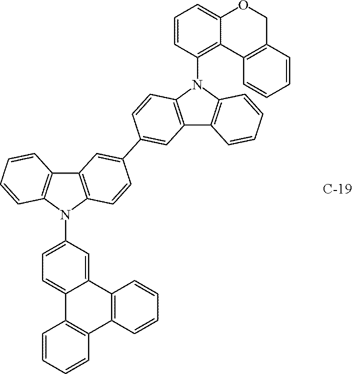 [SMILES: c1ccc2c(c1)COc1cccc(-n3c4ccccc4c4cc(-c5ccc6c(c5)c5ccccc5n6-c5ccc6c7ccccc7c7ccccc7c6c5)ccc43)c1-2] 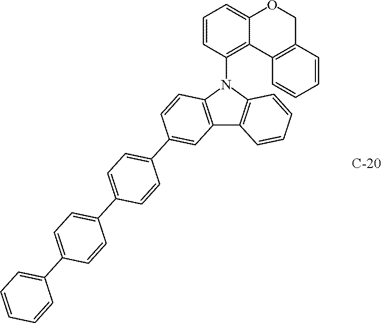 [SMILES: c1ccc(-c2ccc(-c3ccc(-c4ccc5c(c4)c4ccccc4n5-c4cccc5c4-c4ccccc4CO5)cc3)cc2)cc1] 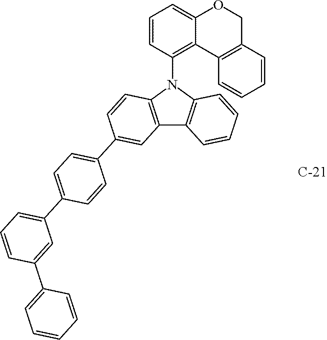 [SMILES: c1ccc(-c2cccc(-c3ccc(-c4ccc5c(c4)c4ccccc4n5-c4cccc5c4-c4ccccc4CO5)cc3)c2)cc1] 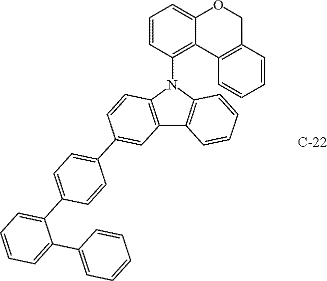 [SMILES: c1ccc(-c2ccccc2-c2ccc(-c3ccc4c(c3)c3ccccc3n4-c3cccc4c3-c3ccccc3CO4)cc2)cc1] 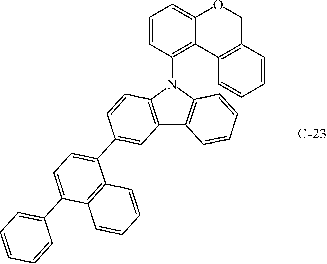 [SMILES: c1ccc(-c2ccc(-c3ccc4c(c3)c3ccccc3n4-c3cccc4c3-c3ccccc3CO4)c3ccccc23)cc1] 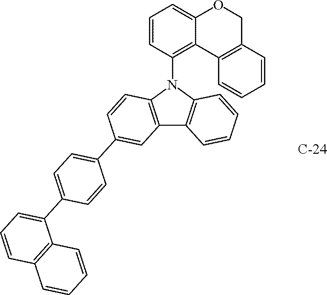 [SMILES: c1ccc2c(c1)COc1cccc(-n3c4ccccc4c4cc(-c5ccc(-c6cccc7ccccc67)cc5)ccc43)c1-2] 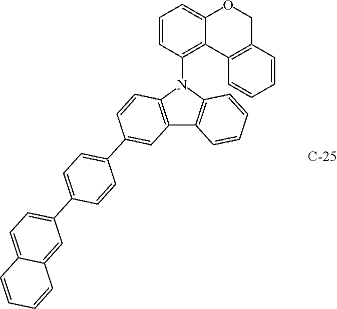 [SMILES: c1ccc2c(c1)COc1cccc(-n3c4ccccc4c4cc(-c5ccc(-c6ccc7ccccc7c6)cc5)ccc43)c1-2] 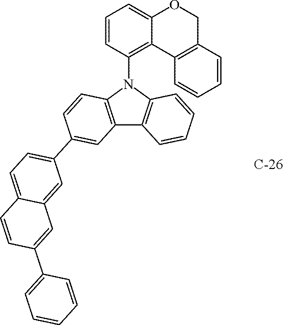 [SMILES: c1ccc(-c2ccc3ccc(-c4ccc5c(c4)c4ccccc4n5-c4cccc5c4-c4ccccc4CO5)cc3c2)cc1] 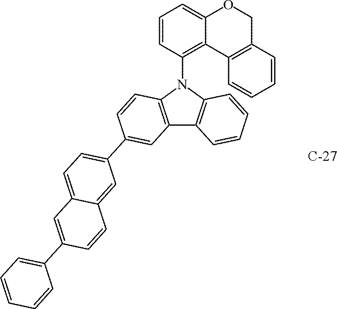 [SMILES: c1ccc(-c2ccc3cc(-c4ccc5c(c4)c4ccccc4n5-c4cccc5c4-c4ccccc4CO5)ccc3c2)cc1] 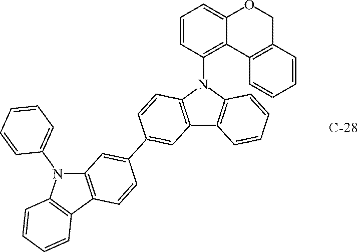 [SMILES: c1ccc(-n2c3ccccc3c3ccc(-c4ccc5c(c4)c4ccccc4n5-c4cccc5c4-c4ccccc4CO5)cc32)cc1] 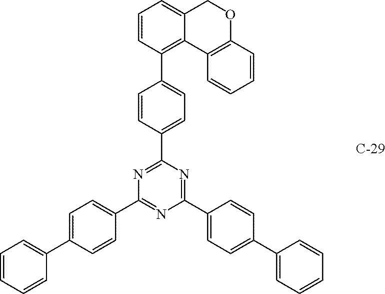 [SMILES: c1ccc(-c2ccc(-c3nc(-c4ccc(-c5ccccc5)cc4)nc(-c4ccc(-c5cccc6c5-c5ccccc5OC6)cc4)n3)cc2)cc1] 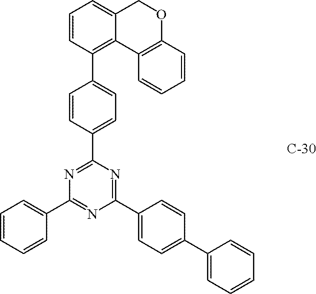 [SMILES: c1ccc(-c2ccc(-c3nc(-c4ccccc4)nc(-c4ccc(-c5cccc6c5-c5ccccc5OC6)cc4)n3)cc2)cc1] 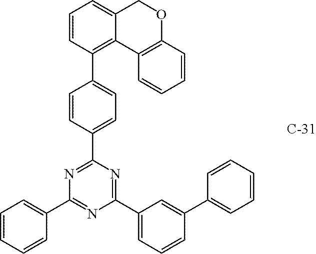 [SMILES: c1ccc(-c2cccc(-c3nc(-c4ccccc4)nc(-c4ccc(-c5cccc6c5-c5ccccc5OC6)cc4)n3)c2)cc1] 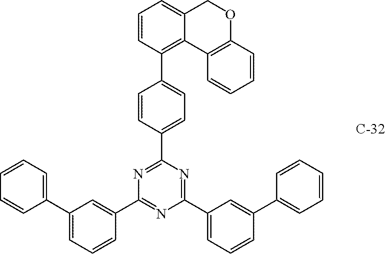 [SMILES: c1ccc(-c2cccc(-c3nc(-c4ccc(-c5cccc6c5-c5ccccc5OC6)cc4)nc(-c4cccc(-c5ccccc5)c4)n3)c2)cc1] 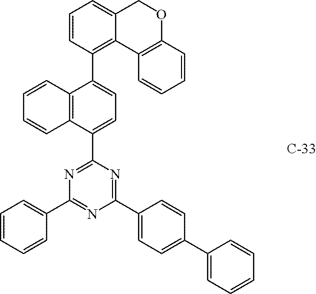 [SMILES: c1ccc(-c2ccc(-c3nc(-c4ccccc4)nc(-c4ccc(-c5cccc6c5-c5ccccc5OC6)c5ccccc45)n3)cc2)cc1] 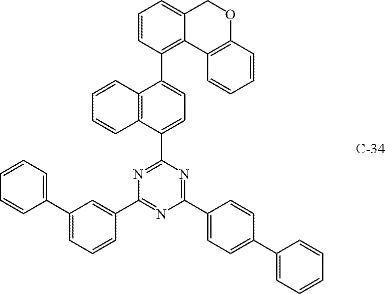 [SMILES: c1ccc(-c2ccc(-c3nc(-c4cccc(-c5ccccc5)c4)nc(-c4ccc(-c5cccc6c5-c5ccccc5OC6)c5ccccc45)n3)cc2)cc1] 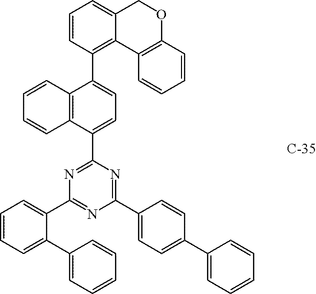 [SMILES: c1ccc(-c2ccc(-c3nc(-c4ccccc4-c4ccccc4)nc(-c4ccc(-c5cccc6c5-c5ccccc5OC6)c5ccccc45)n3)cc2)cc1] 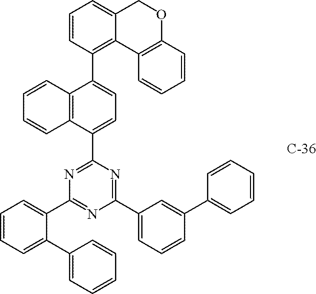 [SMILES: c1ccc(-c2cccc(-c3nc(-c4ccccc4-c4ccccc4)nc(-c4ccc(-c5cccc6c5-c5ccccc5OC6)c5ccccc45)n3)c2)cc1] 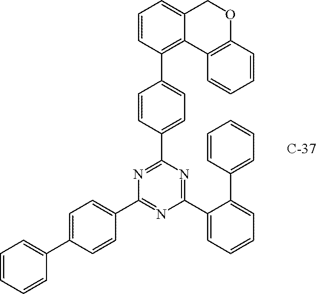 [SMILES: c1ccc(-c2ccc(-c3nc(-c4ccc(-c5cccc6c5-c5ccccc5OC6)cc4)nc(-c4ccccc4-c4ccccc4)n3)cc2)cc1] 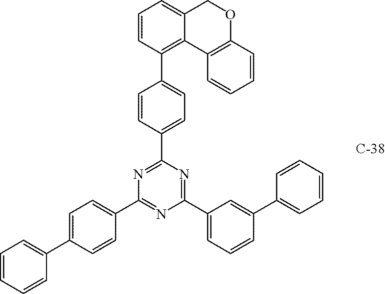 [SMILES: c1ccc(-c2ccc(-c3nc(-c4ccc(-c5cccc6c5-c5ccccc5OC6)cc4)nc(-c4cccc(-c5ccccc5)c4)n3)cc2)cc1] 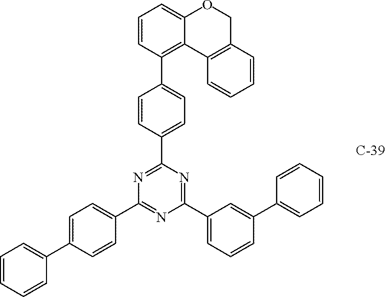 [SMILES: c1ccc(-c2ccc(-c3nc(-c4ccc(-c5cccc6c5-c5ccccc5CO6)cc4)nc(-c4cccc(-c5ccccc5)c4)n3)cc2)cc1] 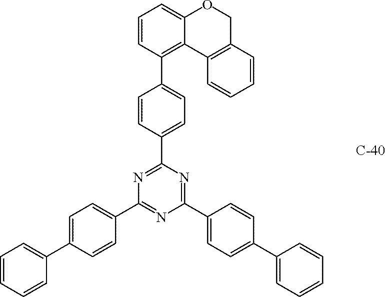 [SMILES: c1ccc(-c2ccc(-c3nc(-c4ccc(-c5ccccc5)cc4)nc(-c4ccc(-c5cccc6c5-c5ccccc5CO6)cc4)n3)cc2)cc1] 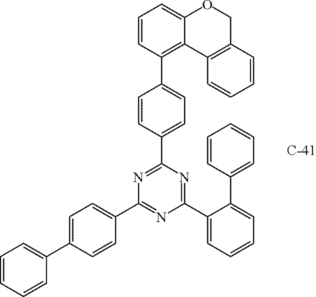 [SMILES: c1ccc(-c2ccc(-c3nc(-c4ccc(-c5cccc6c5-c5ccccc5CO6)cc4)nc(-c4ccccc4-c4ccccc4)n3)cc2)cc1] 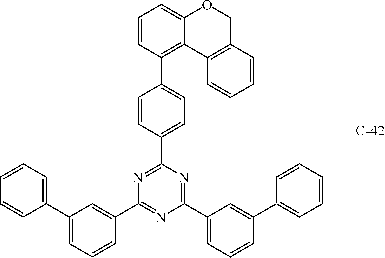 [SMILES: c1ccc(-c2cccc(-c3nc(-c4ccc(-c5cccc6c5-c5ccccc5CO6)cc4)nc(-c4cccc(-c5ccccc5)c4)n3)c2)cc1] 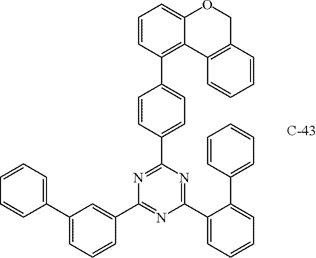 [SMILES: c1ccc(-c2cccc(-c3nc(-c4ccc(-c5cccc6c5-c5ccccc5CO6)cc4)nc(-c4ccccc4-c4ccccc4)n3)c2)cc1] 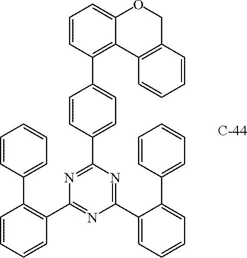 [SMILES: c1ccc(-c2ccccc2-c2nc(-c3ccc(-c4cccc5c4-c4ccccc4CO5)cc3)nc(-c3ccccc3-c3ccccc3)n2)cc1] 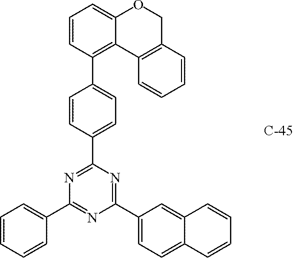 [SMILES: c1ccc(-c2nc(-c3ccc(-c4cccc5c4-c4ccccc4CO5)cc3)nc(-c3ccc4ccccc4c3)n2)cc1] 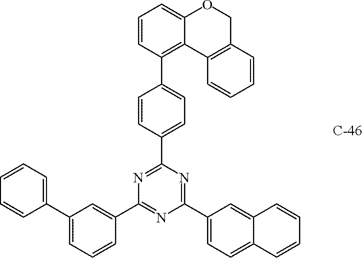 [SMILES: c1ccc(-c2cccc(-c3nc(-c4ccc(-c5cccc6c5-c5ccccc5CO6)cc4)nc(-c4ccc5ccccc5c4)n3)c2)cc1] 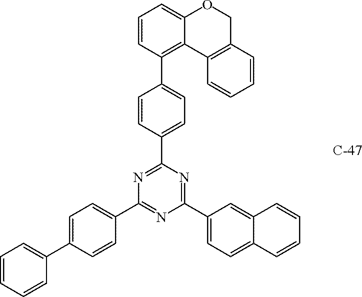 [SMILES: c1ccc(-c2ccc(-c3nc(-c4ccc(-c5cccc6c5-c5ccccc5CO6)cc4)nc(-c4ccc5ccccc5c4)n3)cc2)cc1] 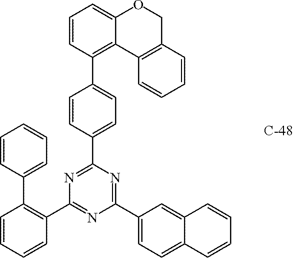 [SMILES: c1ccc(-c2ccccc2-c2nc(-c3ccc(-c4cccc5c4-c4ccccc4CO5)cc3)nc(-c3ccc4ccccc4c3)n2)cc1] 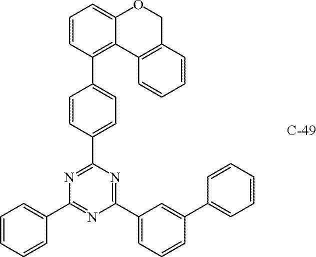 [SMILES: c1ccc(-c2cccc(-c3nc(-c4ccccc4)nc(-c4ccc(-c5cccc6c5-c5ccccc5CO6)cc4)n3)c2)cc1] 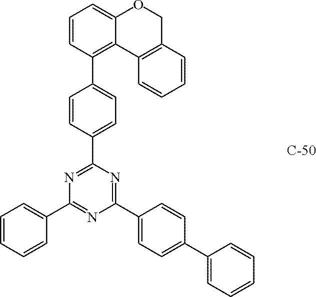 [SMILES: c1ccc(-c2ccc(-c3nc(-c4ccccc4)nc(-c4ccc(-c5cccc6c5-c5ccccc5CO6)cc4)n3)cc2)cc1] 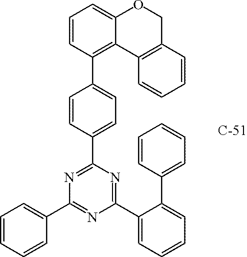 [SMILES: c1ccc(-c2nc(-c3ccc(-c4cccc5c4-c4ccccc4CO5)cc3)nc(-c3ccccc3-c3ccccc3)n2)cc1] 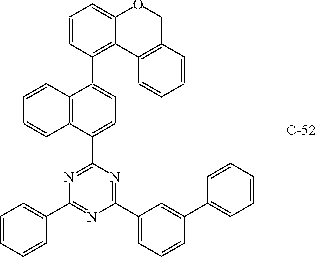 [SMILES: c1ccc(-c2cccc(-c3nc(-c4ccccc4)nc(-c4ccc(-c5cccc6c5-c5ccccc5CO6)c5ccccc45)n3)c2)cc1] 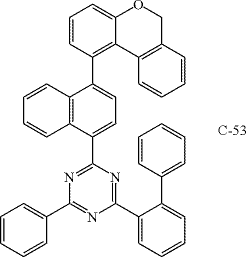 [SMILES: c1ccc(-c2nc(-c3ccccc3-c3ccccc3)nc(-c3ccc(-c4cccc5c4-c4ccccc4CO5)c4ccccc34)n2)cc1] 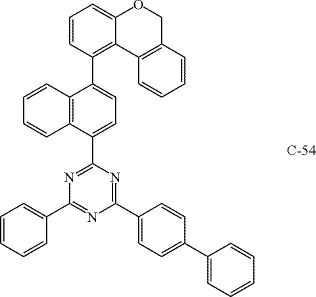 [SMILES: c1ccc(-c2ccc(-c3nc(-c4ccccc4)nc(-c4ccc(-c5cccc6c5-c5ccccc5CO6)c5ccccc45)n3)cc2)cc1] 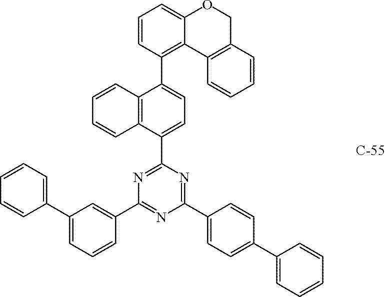 [SMILES: c1ccc(-c2ccc(-c3nc(-c4cccc(-c5ccccc5)c4)nc(-c4ccc(-c5cccc6c5-c5ccccc5CO6)c5ccccc45)n3)cc2)cc1] 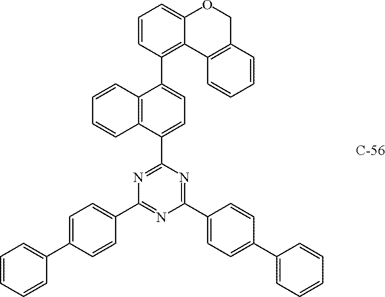 [SMILES: c1ccc(-c2ccc(-c3nc(-c4ccc(-c5ccccc5)cc4)nc(-c4ccc(-c5cccc6c5-c5ccccc5CO6)c5ccccc45)n3)cc2)cc1] 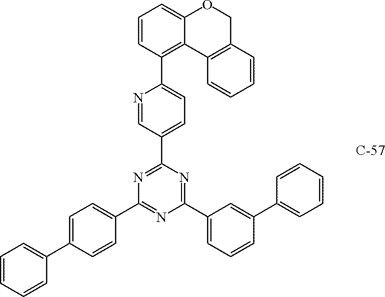 [SMILES: c1ccc(-c2ccc(-c3nc(-c4ccc(-c5cccc6c5-c5ccccc5CO6)nc4)nc(-c4cccc(-c5ccccc5)c4)n3)cc2)cc1] 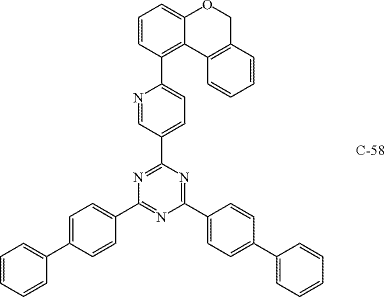 [SMILES: c1ccc(-c2ccc(-c3nc(-c4ccc(-c5ccccc5)cc4)nc(-c4ccc(-c5cccc6c5-c5ccccc5CO6)nc4)n3)cc2)cc1] 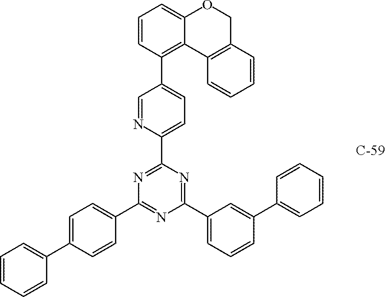 [SMILES: c1ccc(-c2ccc(-c3nc(-c4cccc(-c5ccccc5)c4)nc(-c4ccc(-c5cccc6c5-c5ccccc5CO6)cn4)n3)cc2)cc1] 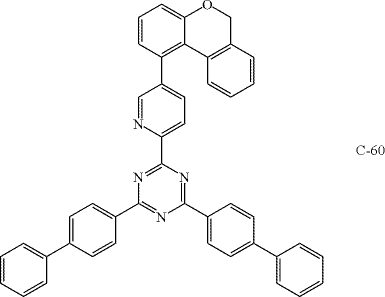 [SMILES: c1ccc(-c2ccc(-c3nc(-c4ccc(-c5ccccc5)cc4)nc(-c4ccc(-c5cccc6c5-c5ccccc5CO6)cn4)n3)cc2)cc1] 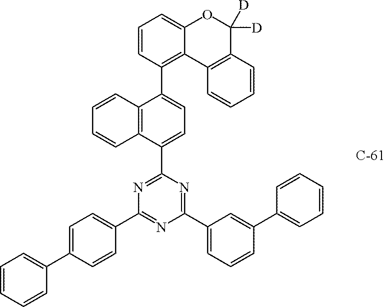 [SMILES: [2H]C1([2H])Oc2cccc(-c3ccc(-c4nc(-c5ccc(-c6ccccc6)cc5)nc(-c5cccc(-c6ccccc6)c5)n4)c4ccccc34)c2-c2ccccc21] 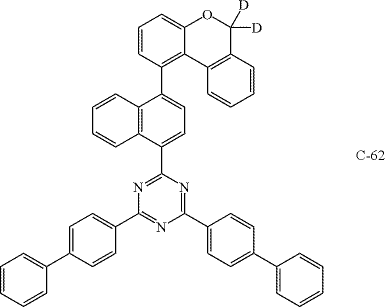 [SMILES: [2H]C1([2H])Oc2cccc(-c3ccc(-c4nc(-c5ccc(-c6ccccc6)cc5)nc(-c5ccc(-c6ccccc6)cc5)n4)c4ccccc34)c2-c2ccccc21] 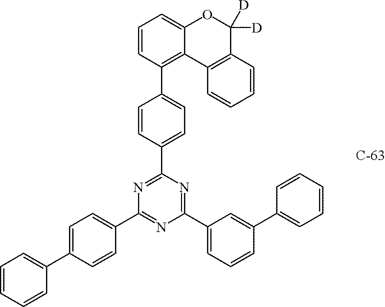 [SMILES: [2H]C1([2H])Oc2cccc(-c3ccc(-c4nc(-c5ccc(-c6ccccc6)cc5)nc(-c5cccc(-c6ccccc6)c5)n4)cc3)c2-c2ccccc21] 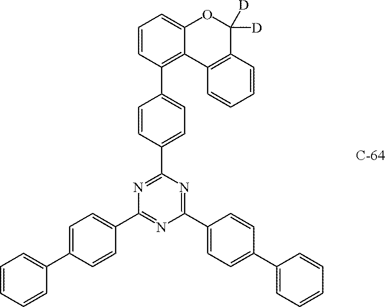 [SMILES: [2H]C1([2H])Oc2cccc(-c3ccc(-c4nc(-c5ccc(-c6ccccc6)cc5)nc(-c5ccc(-c6ccccc6)cc5)n4)cc3)c2-c2ccccc21] 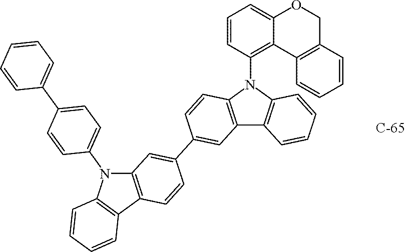 [SMILES: c1ccc(-c2ccc(-n3c4ccccc4c4ccc(-c5ccc6c(c5)c5ccccc5n6-c5cccc6c5-c5ccccc5CO6)cc43)cc2)cc1] 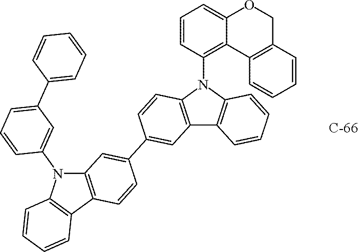 [SMILES: c1ccc(-c2cccc(-n3c4ccccc4c4ccc(-c5ccc6c(c5)c5ccccc5n6-c5cccc6c5-c5ccccc5CO6)cc43)c2)cc1] 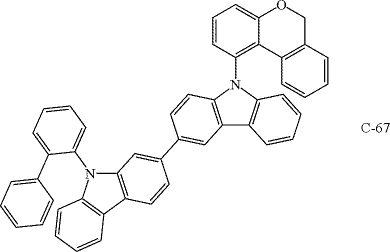 [SMILES: c1ccc(-c2ccccc2-n2c3ccccc3c3ccc(-c4ccc5c(c4)c4ccccc4n5-c4cccc5c4-c4ccccc4CO5)cc32)cc1] 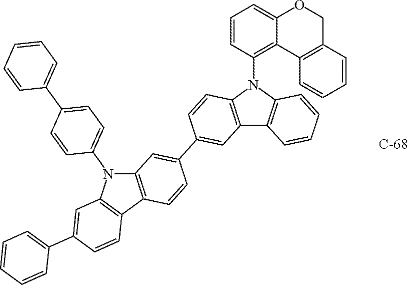 [SMILES: c1ccc(-c2ccc(-n3c4cc(-c5ccccc5)ccc4c4ccc(-c5ccc6c(c5)c5ccccc5n6-c5cccc6c5-c5ccccc5CO6)cc43)cc2)cc1] 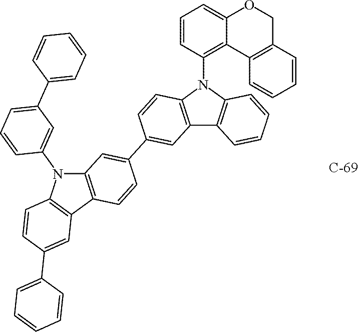 [SMILES: c1ccc(-c2cccc(-n3c4ccc(-c5ccccc5)cc4c4ccc(-c5ccc6c(c5)c5ccccc5n6-c5cccc6c5-c5ccccc5CO6)cc43)c2)cc1] 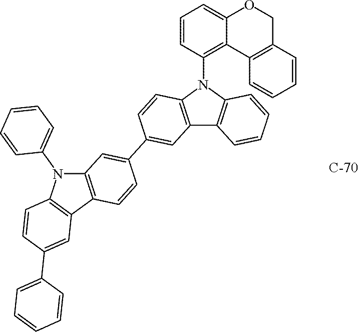 [SMILES: c1ccc(-c2ccc3c(c2)c2ccc(-c4ccc5c(c4)c4ccccc4n5-c4cccc5c4-c4ccccc4CO5)cc2n3-c2ccccc2)cc1] 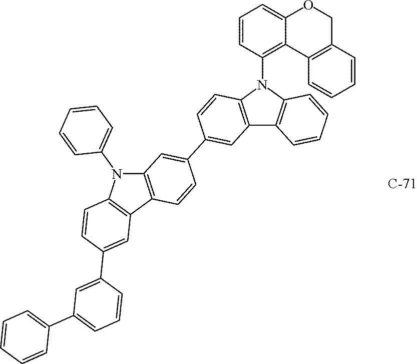 [SMILES: c1ccc(-c2cccc(-c3ccc4c(c3)c3ccc(-c5ccc6c(c5)c5ccccc5n6-c5cccc6c5-c5ccccc5CO6)cc3n4-c3ccccc3)c2)cc1] 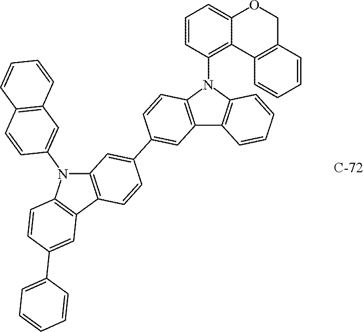 [SMILES: c1ccc(-c2ccc3c(c2)c2ccc(-c4ccc5c(c4)c4ccccc4n5-c4cccc5c4-c4ccccc4CO5)cc2n3-c2ccc3ccccc3c2)cc1] 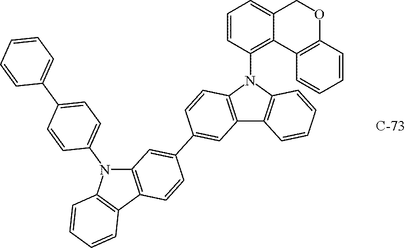 [SMILES: c1ccc(-c2ccc(-n3c4ccccc4c4ccc(-c5ccc6c(c5)c5ccccc5n6-c5cccc6c5-c5ccccc5OC6)cc43)cc2)cc1] 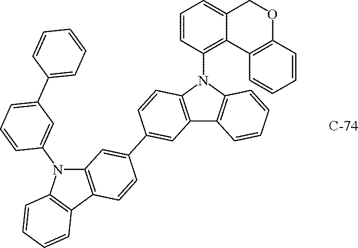 [SMILES: c1ccc(-c2cccc(-n3c4ccccc4c4ccc(-c5ccc6c(c5)c5ccccc5n6-c5cccc6c5-c5ccccc5OC6)cc43)c2)cc1] 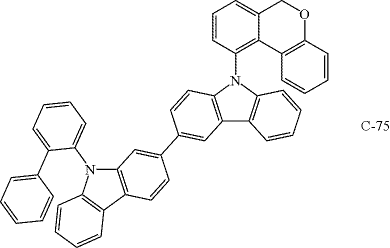 [SMILES: c1ccc(-c2ccccc2-n2c3ccccc3c3ccc(-c4ccc5c(c4)c4ccccc4n5-c4cccc5c4-c4ccccc4OC5)cc32)cc1] 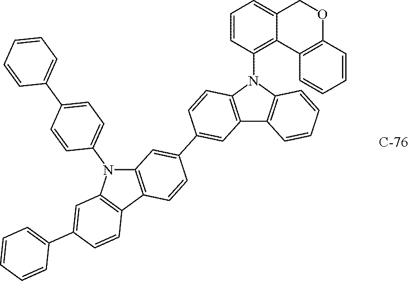 [SMILES: c1ccc(-c2ccc(-n3c4cc(-c5ccccc5)ccc4c4ccc(-c5ccc6c(c5)c5ccccc5n6-c5cccc6c5-c5ccccc5OC6)cc43)cc2)cc1] 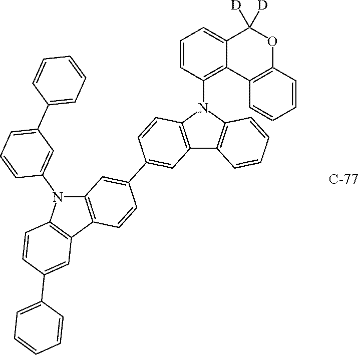 [SMILES: [2H]C1([2H])Oc2ccccc2-c2c(-n3c4ccccc4c4cc(-c5ccc6c7cc(-c8ccccc8)ccc7n(-c7cccc(-c8ccccc8)c7)c6c5)ccc43)cccc21] 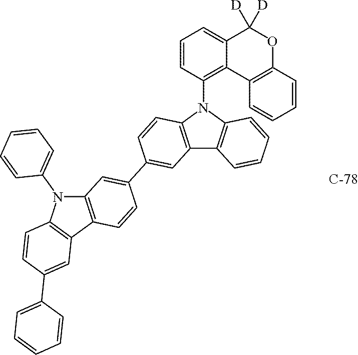 [SMILES: [2H]C1([2H])Oc2ccccc2-c2c(-n3c4ccccc4c4cc(-c5ccc6c7cc(-c8ccccc8)ccc7n(-c7ccccc7)c6c5)ccc43)cccc21] 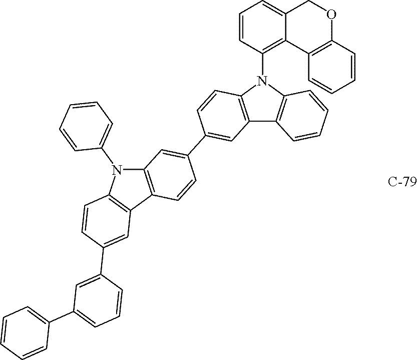 [SMILES: c1ccc(-c2cccc(-c3ccc4c(c3)c3ccc(-c5ccc6c(c5)c5ccccc5n6-c5cccc6c5-c5ccccc5OC6)cc3n4-c3ccccc3)c2)cc1] 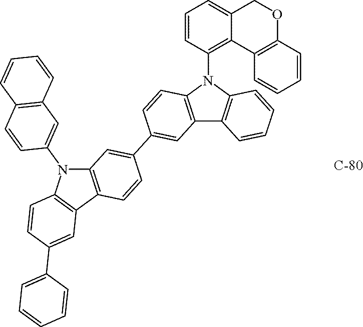 [SMILES: c1ccc(-c2ccc3c(c2)c2ccc(-c4ccc5c(c4)c4ccccc4n5-c4cccc5c4-c4ccccc4OC5)cc2n3-c2ccc3ccccc3c2)cc1] 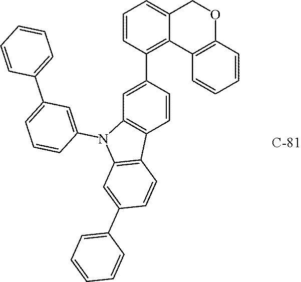 [SMILES: c1ccc(-c2cccc(-n3c4cc(-c5ccccc5)ccc4c4ccc(-c5cccc6c5-c5ccccc5OC6)cc43)c2)cc1] 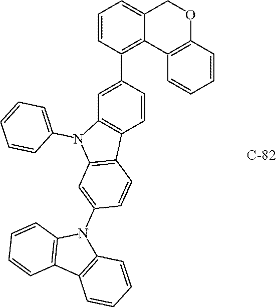 [SMILES: c1ccc(-n2c3cc(-c4cccc5c4-c4ccccc4OC5)ccc3c3ccc(-n4c5ccccc5c5ccccc54)cc32)cc1] 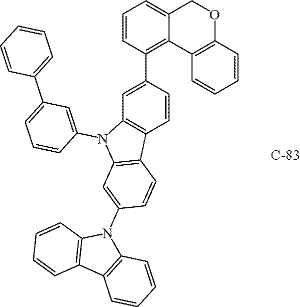 [SMILES: c1ccc(-c2cccc(-n3c4cc(-c5cccc6c5-c5ccccc5OC6)ccc4c4ccc(-n5c6ccccc6c6ccccc65)cc43)c2)cc1] 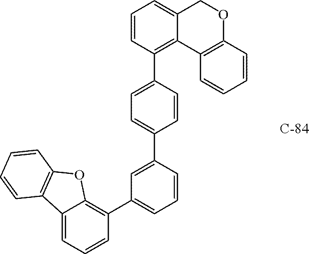 [SMILES: c1cc(-c2ccc(-c3cccc4c3-c3ccccc3OC4)cc2)cc(-c2cccc3c2oc2ccccc23)c1] 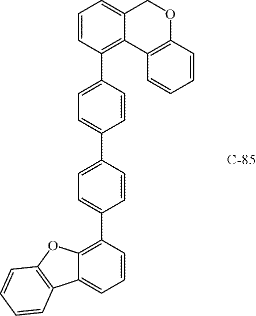 [SMILES: c1ccc2c(c1)OCc1cccc(-c3ccc(-c4ccc(-c5cccc6c5oc5ccccc56)cc4)cc3)c1-2] 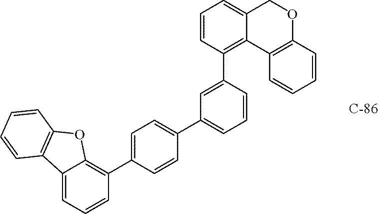 [SMILES: c1cc(-c2ccc(-c3cccc4c3oc3ccccc34)cc2)cc(-c2cccc3c2-c2ccccc2OC3)c1] 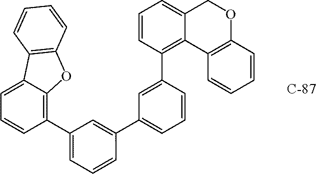 [SMILES: c1cc(-c2cccc(-c3cccc4c3oc3ccccc34)c2)cc(-c2cccc3c2-c2ccccc2OC3)c1] 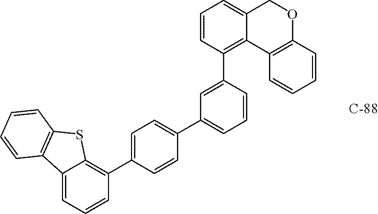 [SMILES: c1cc(-c2ccc(-c3cccc4c3sc3ccccc34)cc2)cc(-c2cccc3c2-c2ccccc2OC3)c1] 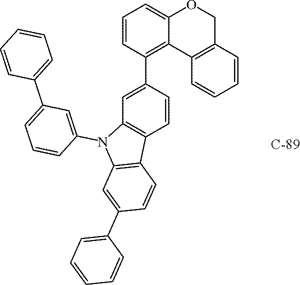 [SMILES: c1ccc(-c2cccc(-n3c4cc(-c5ccccc5)ccc4c4ccc(-c5cccc6c5-c5ccccc5CO6)cc43)c2)cc1] 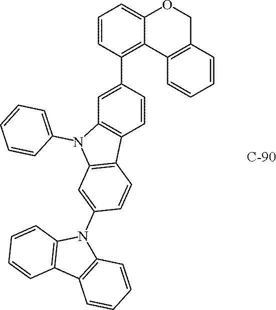 [SMILES: c1ccc(-n2c3cc(-c4cccc5c4-c4ccccc4CO5)ccc3c3ccc(-n4c5ccccc5c5ccccc54)cc32)cc1] 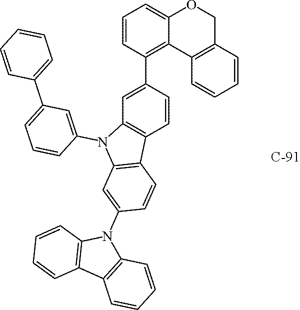 [SMILES: c1ccc(-c2cccc(-n3c4cc(-c5cccc6c5-c5ccccc5CO6)ccc4c4ccc(-n5c6ccccc6c6ccccc65)cc43)c2)cc1] 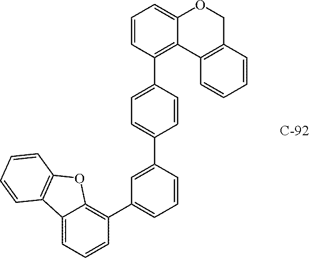 [SMILES: c1cc(-c2ccc(-c3cccc4c3-c3ccccc3CO4)cc2)cc(-c2cccc3c2oc2ccccc23)c1] 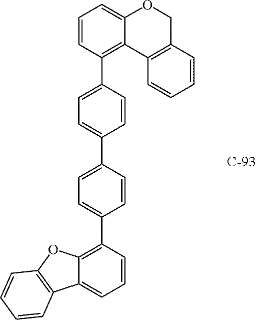 [SMILES: c1ccc2c(c1)COc1cccc(-c3ccc(-c4ccc(-c5cccc6c5oc5ccccc56)cc4)cc3)c1-2] 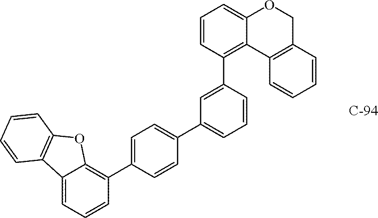 [SMILES: c1cc(-c2ccc(-c3cccc4c3oc3ccccc34)cc2)cc(-c2cccc3c2-c2ccccc2CO3)c1] 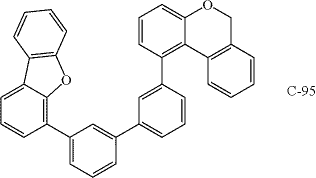 [SMILES: c1cc(-c2cccc(-c3cccc4c3oc3ccccc34)c2)cc(-c2cccc3c2-c2ccccc2CO3)c1] 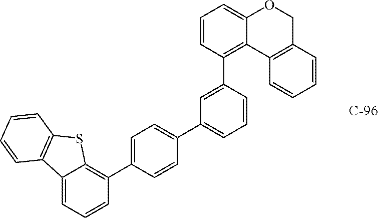 [SMILES: c1cc(-c2ccc(-c3cccc4c3sc3ccccc34)cc2)cc(-c2cccc3c2-c2ccccc2CO3)c1] 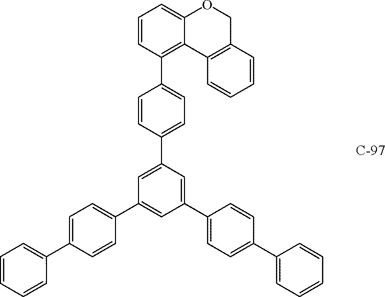 [SMILES: c1ccc(-c2ccc(-c3cc(-c4ccc(-c5ccccc5)cc4)cc(-c4ccc(-c5cccc6c5-c5ccccc5CO6)cc4)c3)cc2)cc1] 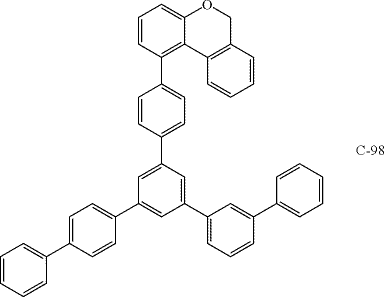 [SMILES: c1ccc(-c2ccc(-c3cc(-c4ccc(-c5cccc6c5-c5ccccc5CO6)cc4)cc(-c4cccc(-c5ccccc5)c4)c3)cc2)cc1] 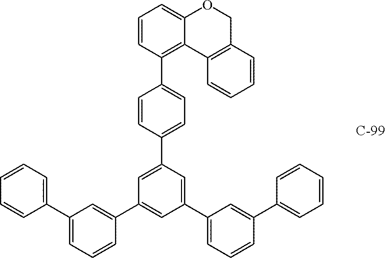 [SMILES: c1ccc(-c2cccc(-c3cc(-c4ccc(-c5cccc6c5-c5ccccc5CO6)cc4)cc(-c4cccc(-c5ccccc5)c4)c3)c2)cc1] 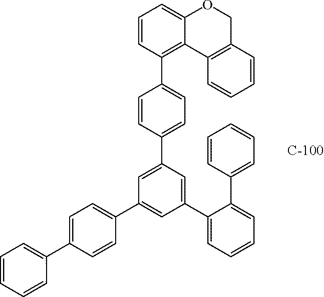 [SMILES: c1ccc(-c2ccc(-c3cc(-c4ccc(-c5cccc6c5-c5ccccc5CO6)cc4)cc(-c4ccccc4-c4ccccc4)c3)cc2)cc1] 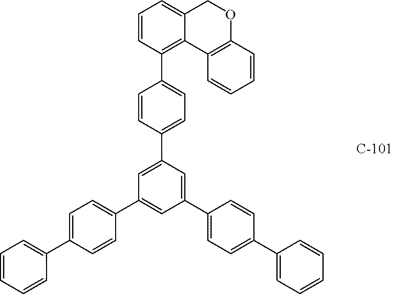 [SMILES: c1ccc(-c2ccc(-c3cc(-c4ccc(-c5ccccc5)cc4)cc(-c4ccc(-c5cccc6c5-c5ccccc5OC6)cc4)c3)cc2)cc1] 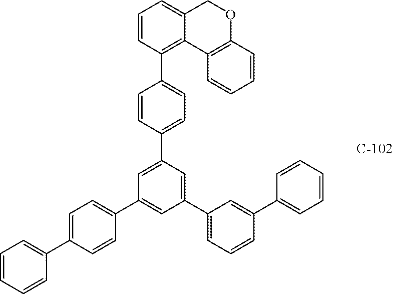 [SMILES: c1ccc(-c2ccc(-c3cc(-c4ccc(-c5cccc6c5-c5ccccc5OC6)cc4)cc(-c4cccc(-c5ccccc5)c4)c3)cc2)cc1] 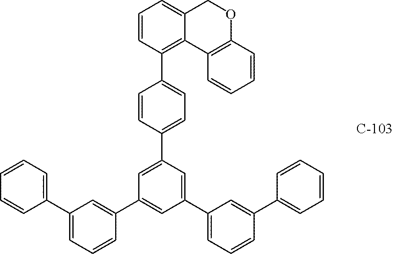 [SMILES: c1ccc(-c2cccc(-c3cc(-c4ccc(-c5cccc6c5-c5ccccc5OC6)cc4)cc(-c4cccc(-c5ccccc5)c4)c3)c2)cc1] 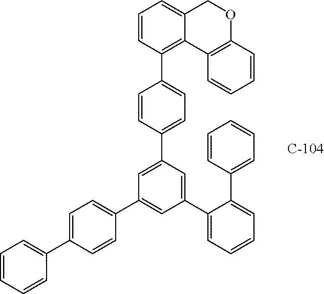 [SMILES: c1ccc(-c2ccc(-c3cc(-c4ccc(-c5cccc6c5-c5ccccc5OC6)cc4)cc(-c4ccccc4-c4ccccc4)c3)cc2)cc1] 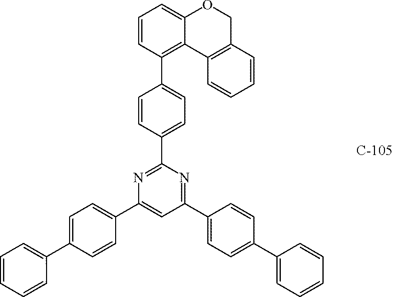 [SMILES: c1ccc(-c2ccc(-c3cc(-c4ccc(-c5ccccc5)cc4)nc(-c4ccc(-c5cccc6c5-c5ccccc5CO6)cc4)n3)cc2)cc1] 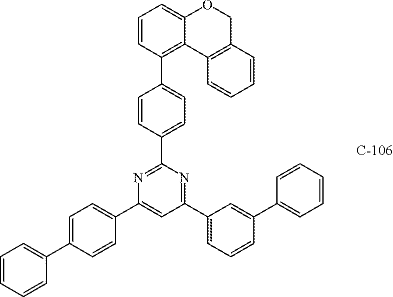 [SMILES: c1ccc(-c2ccc(-c3cc(-c4cccc(-c5ccccc5)c4)nc(-c4ccc(-c5cccc6c5-c5ccccc5CO6)cc4)n3)cc2)cc1] 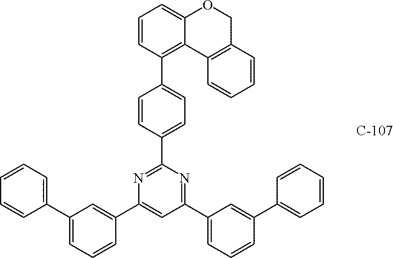 [SMILES: c1ccc(-c2cccc(-c3cc(-c4cccc(-c5ccccc5)c4)nc(-c4ccc(-c5cccc6c5-c5ccccc5CO6)cc4)n3)c2)cc1] 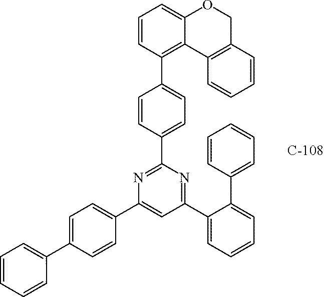 [SMILES: c1ccc(-c2ccc(-c3cc(-c4ccccc4-c4ccccc4)nc(-c4ccc(-c5cccc6c5-c5ccccc5CO6)cc4)n3)cc2)cc1] 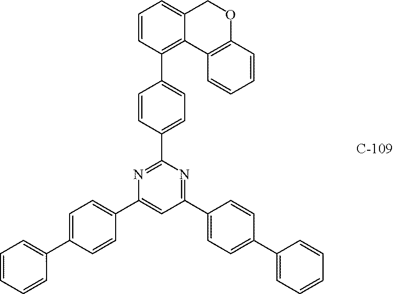 [SMILES: c1ccc(-c2ccc(-c3cc(-c4ccc(-c5ccccc5)cc4)nc(-c4ccc(-c5cccc6c5-c5ccccc5OC6)cc4)n3)cc2)cc1] 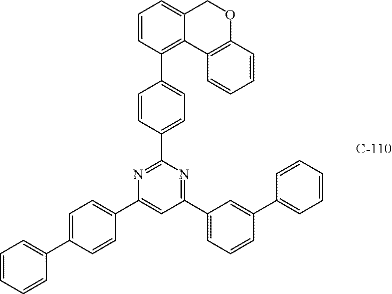 [SMILES: c1ccc(-c2ccc(-c3cc(-c4cccc(-c5ccccc5)c4)nc(-c4ccc(-c5cccc6c5-c5ccccc5OC6)cc4)n3)cc2)cc1] 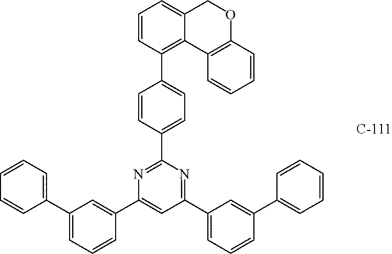 [SMILES: c1ccc(-c2cccc(-c3cc(-c4cccc(-c5ccccc5)c4)nc(-c4ccc(-c5cccc6c5-c5ccccc5OC6)cc4)n3)c2)cc1] 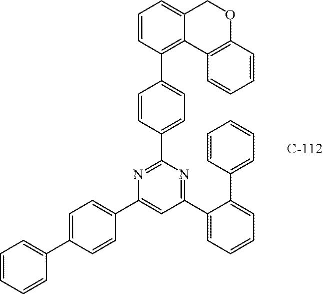 [SMILES: c1ccc(-c2ccc(-c3cc(-c4ccccc4-c4ccccc4)nc(-c4ccc(-c5cccc6c5-c5ccccc5OC6)cc4)n3)cc2)cc1] 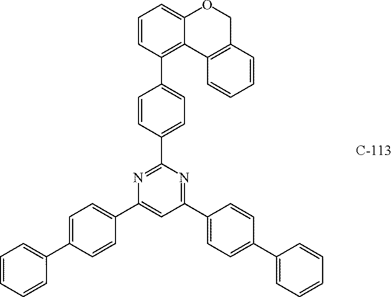 [SMILES: c1ccc(-c2ccc(-c3cc(-c4ccc(-c5ccccc5)cc4)nc(-c4ccc(-c5cccc6c5-c5ccccc5CO6)cc4)n3)cc2)cc1] 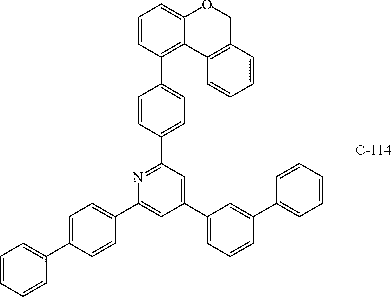 [SMILES: c1ccc(-c2ccc(-c3cc(-c4cccc(-c5ccccc5)c4)cc(-c4ccc(-c5cccc6c5-c5ccccc5CO6)cc4)n3)cc2)cc1] 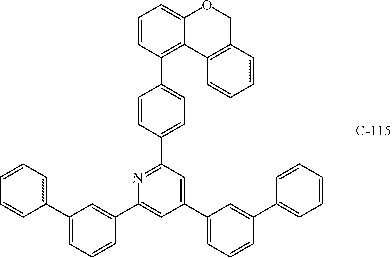 [SMILES: c1ccc(-c2cccc(-c3cc(-c4ccc(-c5cccc6c5-c5ccccc5CO6)cc4)nc(-c4cccc(-c5ccccc5)c4)c3)c2)cc1] 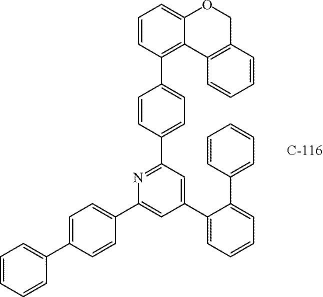 [SMILES: c1ccc(-c2ccc(-c3cc(-c4ccccc4-c4ccccc4)cc(-c4ccc(-c5cccc6c5-c5ccccc5CO6)cc4)n3)cc2)cc1] 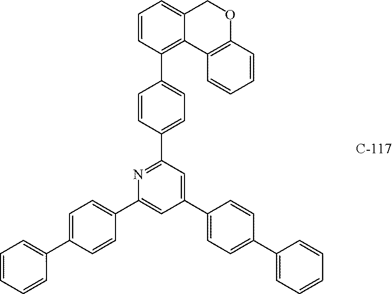 [SMILES: c1ccc(-c2ccc(-c3cc(-c4ccc(-c5ccccc5)cc4)nc(-c4ccc(-c5cccc6c5-c5ccccc5OC6)cc4)c3)cc2)cc1] 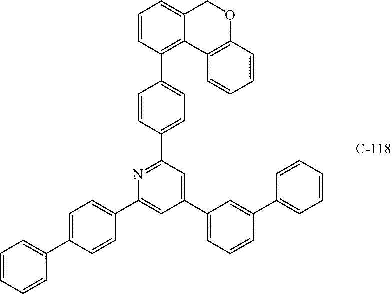 [SMILES: c1ccc(-c2ccc(-c3cc(-c4cccc(-c5ccccc5)c4)cc(-c4ccc(-c5cccc6c5-c5ccccc5OC6)cc4)n3)cc2)cc1] 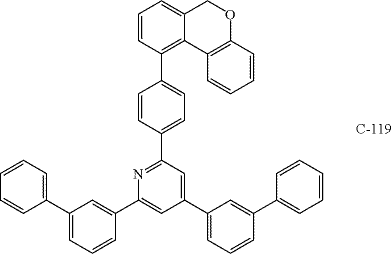 [SMILES: c1ccc(-c2cccc(-c3cc(-c4ccc(-c5cccc6c5-c5ccccc5OC6)cc4)nc(-c4cccc(-c5ccccc5)c4)c3)c2)cc1] 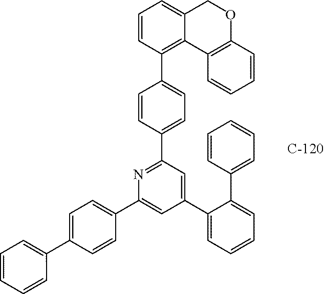 [SMILES: c1ccc(-c2ccc(-c3cc(-c4ccccc4-c4ccccc4)cc(-c4ccc(-c5cccc6c5-c5ccccc5OC6)cc4)n3)cc2)cc1] 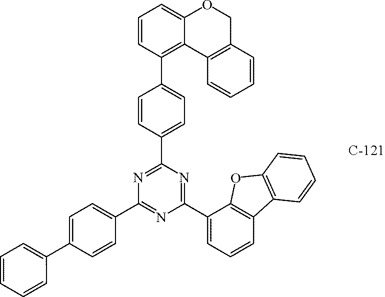 [SMILES: c1ccc(-c2ccc(-c3nc(-c4ccc(-c5cccc6c5-c5ccccc5CO6)cc4)nc(-c4cccc5c4oc4ccccc45)n3)cc2)cc1] 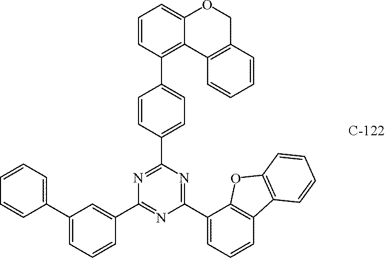 [SMILES: c1ccc(-c2cccc(-c3nc(-c4ccc(-c5cccc6c5-c5ccccc5CO6)cc4)nc(-c4cccc5c4oc4ccccc45)n3)c2)cc1] 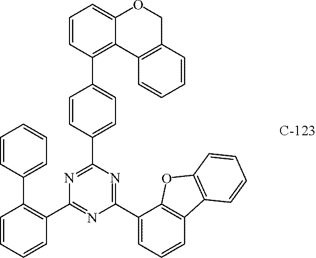 [SMILES: c1ccc(-c2ccccc2-c2nc(-c3ccc(-c4cccc5c4-c4ccccc4CO5)cc3)nc(-c3cccc4c3oc3ccccc34)n2)cc1] 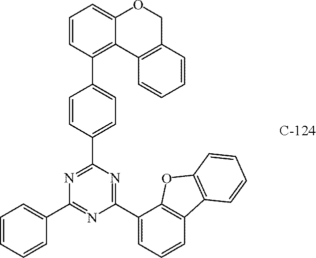 [SMILES: c1ccc(-c2nc(-c3ccc(-c4cccc5c4-c4ccccc4CO5)cc3)nc(-c3cccc4c3oc3ccccc34)n2)cc1] 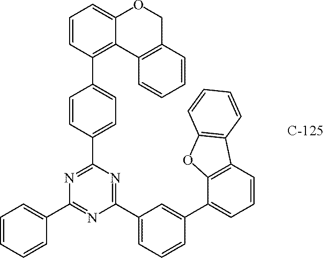 [SMILES: c1ccc(-c2nc(-c3ccc(-c4cccc5c4-c4ccccc4CO5)cc3)nc(-c3cccc(-c4cccc5c4oc4ccccc45)c3)n2)cc1] 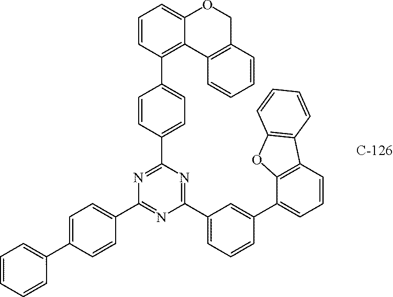 [SMILES: c1ccc(-c2ccc(-c3nc(-c4ccc(-c5cccc6c5-c5ccccc5CO6)cc4)nc(-c4cccc(-c5cccc6c5oc5ccccc56)c4)n3)cc2)cc1] 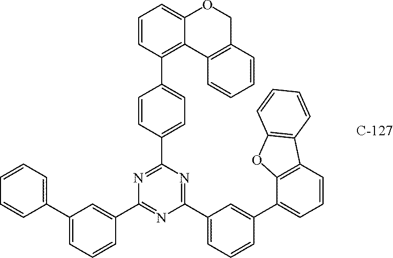 [SMILES: c1ccc(-c2cccc(-c3nc(-c4ccc(-c5cccc6c5-c5ccccc5CO6)cc4)nc(-c4cccc(-c5cccc6c5oc5ccccc56)c4)n3)c2)cc1] 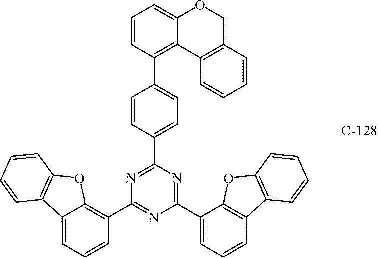 [SMILES: c1ccc2c(c1)COc1cccc(-c3ccc(-c4nc(-c5cccc6c5oc5ccccc56)nc(-c5cccc6c5oc5ccccc56)n4)cc3)c1-2] 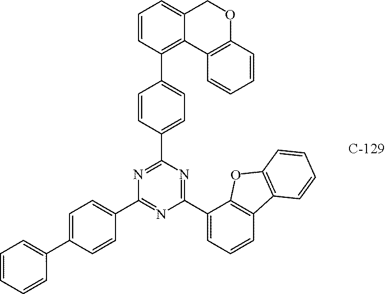 [SMILES: c1ccc(-c2ccc(-c3nc(-c4ccc(-c5cccc6c5-c5ccccc5OC6)cc4)nc(-c4cccc5c4oc4ccccc45)n3)cc2)cc1] 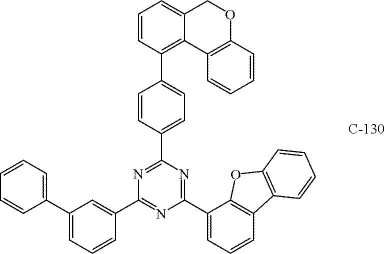 [SMILES: c1ccc(-c2cccc(-c3nc(-c4ccc(-c5cccc6c5-c5ccccc5OC6)cc4)nc(-c4cccc5c4oc4ccccc45)n3)c2)cc1] 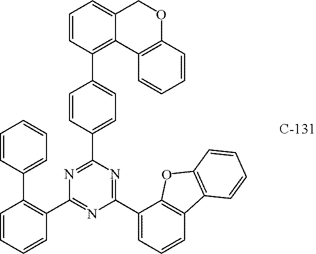 [SMILES: c1ccc(-c2ccccc2-c2nc(-c3ccc(-c4cccc5c4-c4ccccc4OC5)cc3)nc(-c3cccc4c3oc3ccccc34)n2)cc1] 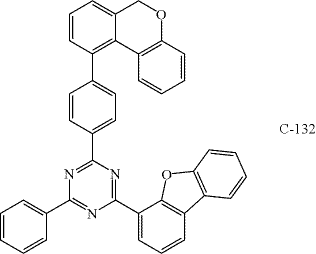 [SMILES: c1ccc(-c2nc(-c3ccc(-c4cccc5c4-c4ccccc4OC5)cc3)nc(-c3cccc4c3oc3ccccc34)n2)cc1] 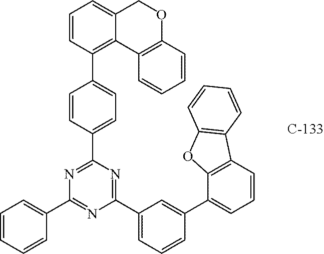 [SMILES: c1ccc(-c2nc(-c3ccc(-c4cccc5c4-c4ccccc4OC5)cc3)nc(-c3cccc(-c4cccc5c4oc4ccccc45)c3)n2)cc1] 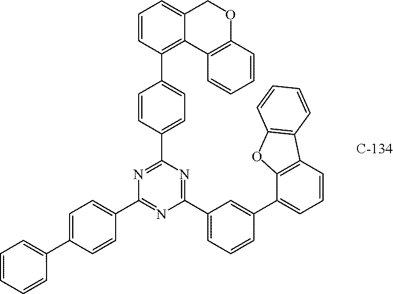 [SMILES: c1ccc(-c2ccc(-c3nc(-c4ccc(-c5cccc6c5-c5ccccc5OC6)cc4)nc(-c4cccc(-c5cccc6c5oc5ccccc56)c4)n3)cc2)cc1] 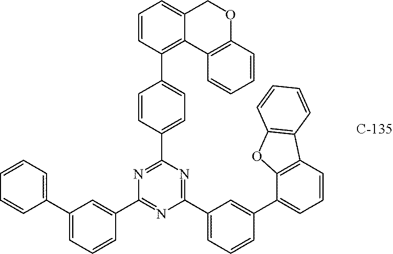 [SMILES: c1ccc(-c2cccc(-c3nc(-c4ccc(-c5cccc6c5-c5ccccc5OC6)cc4)nc(-c4cccc(-c5cccc6c5oc5ccccc56)c4)n3)c2)cc1] 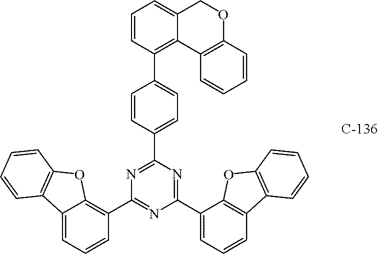 [SMILES: c1ccc2c(c1)OCc1cccc(-c3ccc(-c4nc(-c5cccc6c5oc5ccccc56)nc(-c5cccc6c5oc5ccccc56)n4)cc3)c1-2] 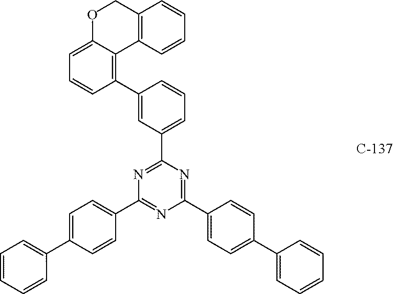 [SMILES: c1ccc(-c2ccc(-c3nc(-c4ccc(-c5ccccc5)cc4)nc(-c4cccc(-c5cccc6c5-c5ccccc5CO6)c4)n3)cc2)cc1] 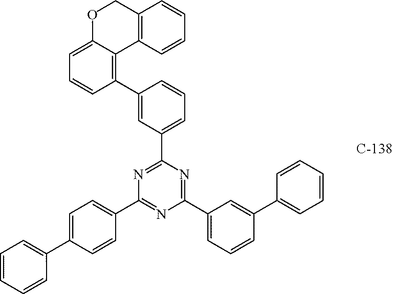 [SMILES: c1ccc(-c2ccc(-c3nc(-c4cccc(-c5ccccc5)c4)nc(-c4cccc(-c5cccc6c5-c5ccccc5CO6)c4)n3)cc2)cc1] 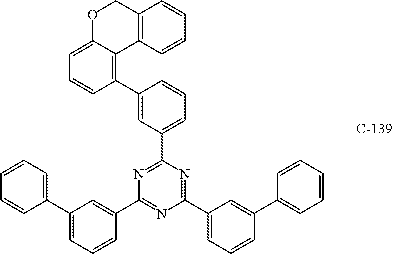 [SMILES: c1ccc(-c2cccc(-c3nc(-c4cccc(-c5ccccc5)c4)nc(-c4cccc(-c5cccc6c5-c5ccccc5CO6)c4)n3)c2)cc1] 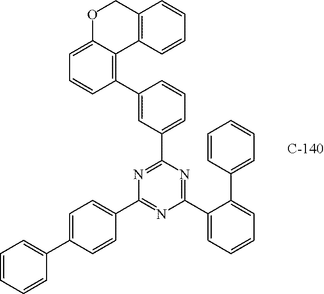 [SMILES: c1ccc(-c2ccc(-c3nc(-c4cccc(-c5cccc6c5-c5ccccc5CO6)c4)nc(-c4ccccc4-c4ccccc4)n3)cc2)cc1] 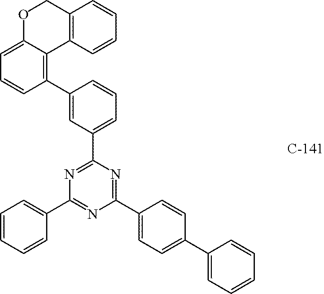 [SMILES: c1ccc(-c2ccc(-c3nc(-c4ccccc4)nc(-c4cccc(-c5cccc6c5-c5ccccc5CO6)c4)n3)cc2)cc1] 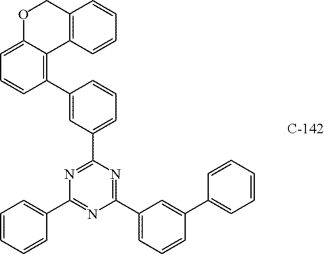 [SMILES: c1ccc(-c2cccc(-c3nc(-c4ccccc4)nc(-c4cccc(-c5cccc6c5-c5ccccc5CO6)c4)n3)c2)cc1] 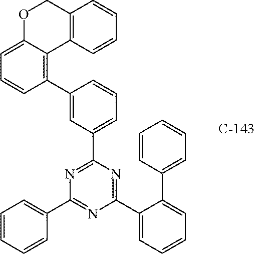 [SMILES: c1ccc(-c2nc(-c3cccc(-c4cccc5c4-c4ccccc4CO5)c3)nc(-c3ccccc3-c3ccccc3)n2)cc1] 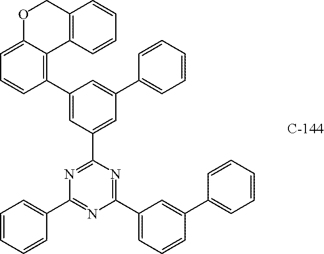 [SMILES: c1ccc(-c2cccc(-c3nc(-c4ccccc4)nc(-c4cc(-c5ccccc5)cc(-c5cccc6c5-c5ccccc5CO6)c4)n3)c2)cc1] 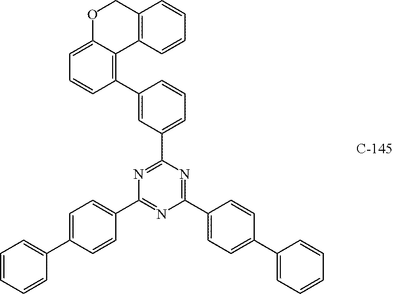 [SMILES: c1ccc(-c2ccc(-c3nc(-c4ccc(-c5ccccc5)cc4)nc(-c4cccc(-c5cccc6c5-c5ccccc5CO6)c4)n3)cc2)cc1] 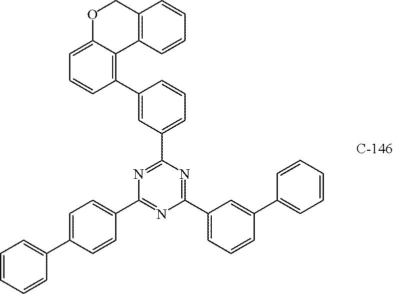 [SMILES: c1ccc(-c2ccc(-c3nc(-c4cccc(-c5ccccc5)c4)nc(-c4cccc(-c5cccc6c5-c5ccccc5CO6)c4)n3)cc2)cc1] 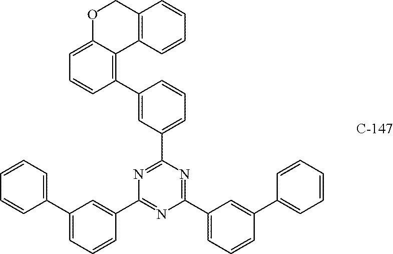 [SMILES: c1ccc(-c2cccc(-c3nc(-c4cccc(-c5ccccc5)c4)nc(-c4cccc(-c5cccc6c5-c5ccccc5CO6)c4)n3)c2)cc1] 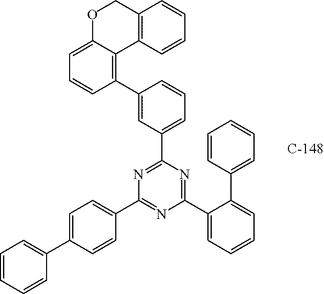 [SMILES: c1ccc(-c2ccc(-c3nc(-c4cccc(-c5cccc6c5-c5ccccc5CO6)c4)nc(-c4ccccc4-c4ccccc4)n3)cc2)cc1] 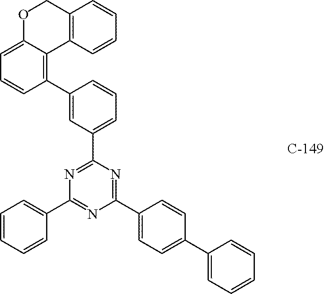 [SMILES: c1ccc(-c2ccc(-c3nc(-c4ccccc4)nc(-c4cccc(-c5cccc6c5-c5ccccc5CO6)c4)n3)cc2)cc1] 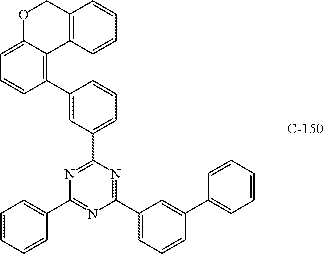 [SMILES: c1ccc(-c2cccc(-c3nc(-c4ccccc4)nc(-c4cccc(-c5cccc6c5-c5ccccc5CO6)c4)n3)c2)cc1] 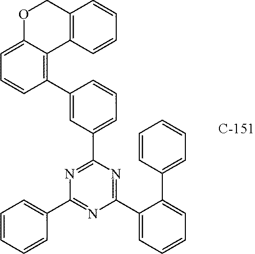 [SMILES: c1ccc(-c2nc(-c3cccc(-c4cccc5c4-c4ccccc4CO5)c3)nc(-c3ccccc3-c3ccccc3)n2)cc1] 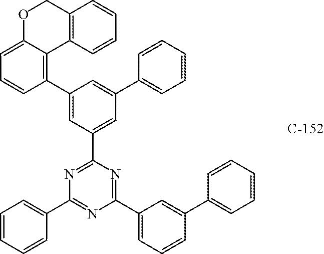 [SMILES: c1ccc(-c2cccc(-c3nc(-c4ccccc4)nc(-c4cc(-c5ccccc5)cc(-c5cccc6c5-c5ccccc5CO6)c4)n3)c2)cc1] 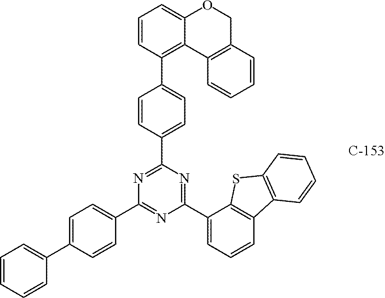 [SMILES: c1ccc(-c2ccc(-c3nc(-c4ccc(-c5cccc6c5-c5ccccc5CO6)cc4)nc(-c4cccc5c4sc4ccccc45)n3)cc2)cc1] 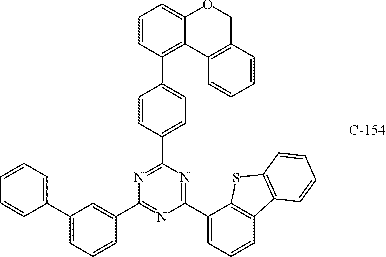 [SMILES: c1ccc(-c2cccc(-c3nc(-c4ccc(-c5cccc6c5-c5ccccc5CO6)cc4)nc(-c4cccc5c4sc4ccccc45)n3)c2)cc1] 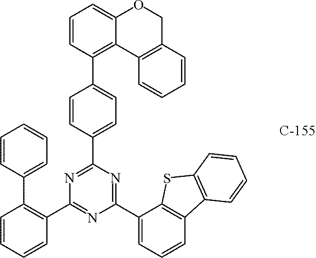 [SMILES: c1ccc(-c2ccccc2-c2nc(-c3ccc(-c4cccc5c4-c4ccccc4CO5)cc3)nc(-c3cccc4c3sc3ccccc34)n2)cc1] 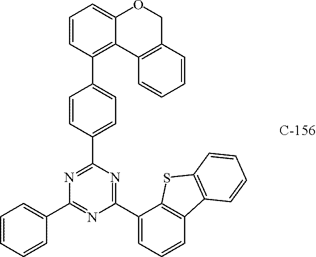 [SMILES: c1ccc(-c2nc(-c3ccc(-c4cccc5c4-c4ccccc4CO5)cc3)nc(-c3cccc4c3sc3ccccc34)n2)cc1] 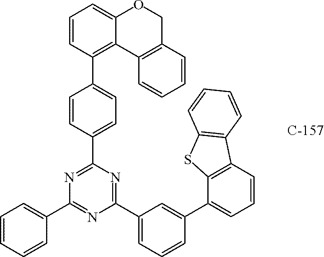 [SMILES: c1ccc(-c2nc(-c3ccc(-c4cccc5c4-c4ccccc4CO5)cc3)nc(-c3cccc(-c4cccc5c4sc4ccccc45)c3)n2)cc1] 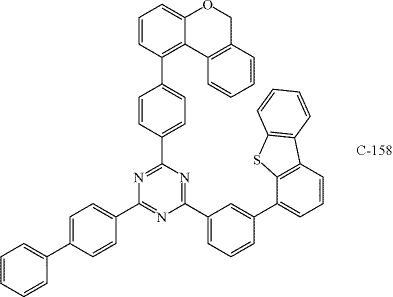 [SMILES: c1ccc(-c2ccc(-c3nc(-c4ccc(-c5cccc6c5-c5ccccc5CO6)cc4)nc(-c4cccc(-c5cccc6c5sc5ccccc56)c4)n3)cc2)cc1] 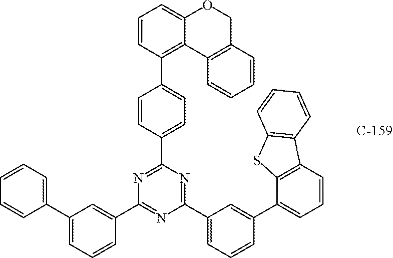 [SMILES: c1ccc(-c2cccc(-c3nc(-c4ccc(-c5cccc6c5-c5ccccc5CO6)cc4)nc(-c4cccc(-c5cccc6c5sc5ccccc56)c4)n3)c2)cc1] 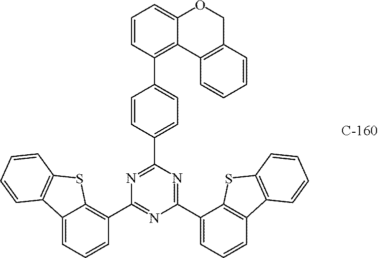 [SMILES: c1ccc2c(c1)COc1cccc(-c3ccc(-c4nc(-c5cccc6c5sc5ccccc56)nc(-c5cccc6c5sc5ccccc56)n4)cc3)c1-2] 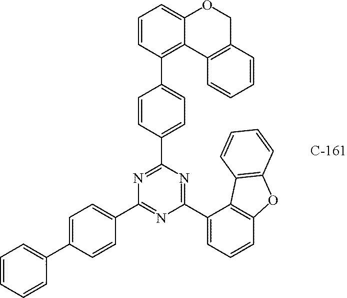 [SMILES: c1ccc(-c2ccc(-c3nc(-c4ccc(-c5cccc6c5-c5ccccc5CO6)cc4)nc(-c4cccc5oc6ccccc6c45)n3)cc2)cc1] 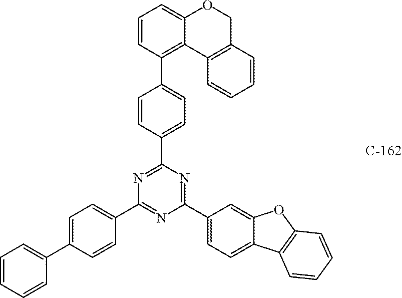 [SMILES: c1ccc(-c2ccc(-c3nc(-c4ccc(-c5cccc6c5-c5ccccc5CO6)cc4)nc(-c4ccc5c(c4)oc4ccccc45)n3)cc2)cc1] 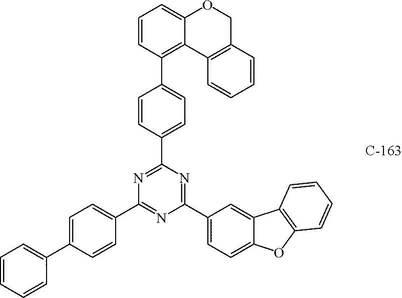 [SMILES: c1ccc(-c2ccc(-c3nc(-c4ccc(-c5cccc6c5-c5ccccc5CO6)cc4)nc(-c4ccc5oc6ccccc6c5c4)n3)cc2)cc1] 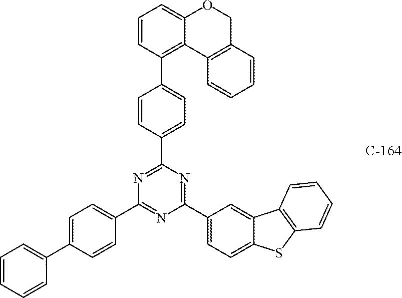 [SMILES: c1ccc(-c2ccc(-c3nc(-c4ccc(-c5cccc6c5-c5ccccc5CO6)cc4)nc(-c4ccc5sc6ccccc6c5c4)n3)cc2)cc1] 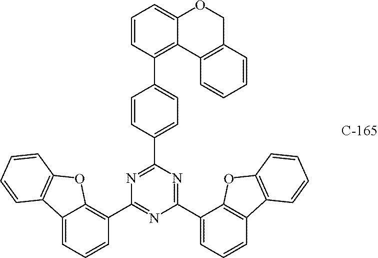 [SMILES: c1ccc2c(c1)COc1cccc(-c3ccc(-c4nc(-c5cccc6c5oc5ccccc56)nc(-c5cccc6c5oc5ccccc56)n4)cc3)c1-2] 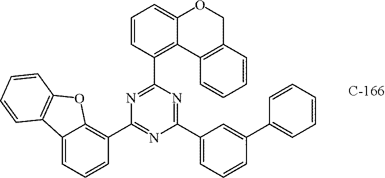 [SMILES: c1ccc(-c2cccc(-c3nc(-c4cccc5c4-c4ccccc4CO5)nc(-c4cccc5c4oc4ccccc45)n3)c2)cc1] 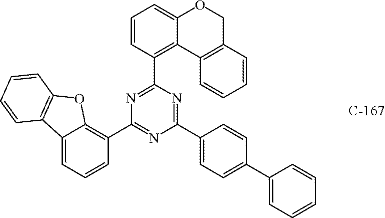 [SMILES: c1ccc(-c2ccc(-c3nc(-c4cccc5c4-c4ccccc4CO5)nc(-c4cccc5c4oc4ccccc45)n3)cc2)cc1] 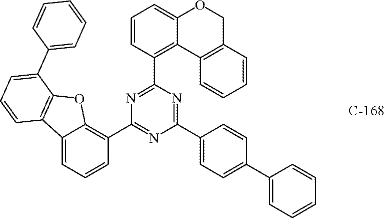 [SMILES: c1ccc(-c2ccc(-c3nc(-c4cccc5c4-c4ccccc4CO5)nc(-c4cccc5c4oc4c(-c6ccccc6)cccc45)n3)cc2)cc1] 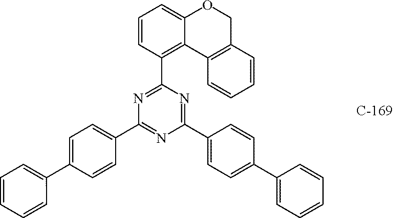 [SMILES: c1ccc(-c2ccc(-c3nc(-c4ccc(-c5ccccc5)cc4)nc(-c4cccc5c4-c4ccccc4CO5)n3)cc2)cc1] 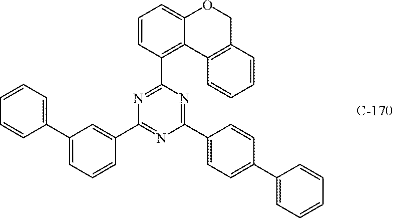 [SMILES: c1ccc(-c2ccc(-c3nc(-c4cccc(-c5ccccc5)c4)nc(-c4cccc5c4-c4ccccc4CO5)n3)cc2)cc1] 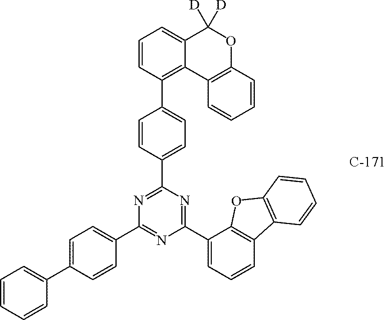 [SMILES: [2H]C1([2H])Oc2ccccc2-c2c(-c3ccc(-c4nc(-c5ccc(-c6ccccc6)cc5)nc(-c5cccc6c5oc5ccccc56)n4)cc3)cccc21] 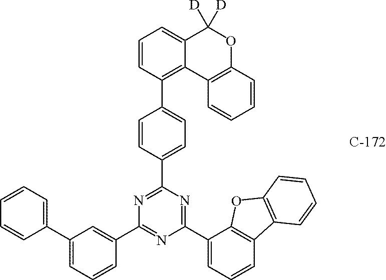 [SMILES: [2H]C1([2H])Oc2ccccc2-c2c(-c3ccc(-c4nc(-c5cccc(-c6ccccc6)c5)nc(-c5cccc6c5oc5ccccc56)n4)cc3)cccc21] 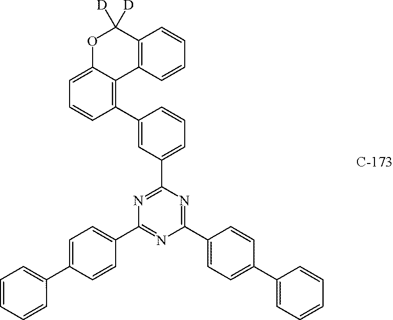 [SMILES: [2H]C1([2H])Oc2cccc(-c3cccc(-c4nc(-c5ccc(-c6ccccc6)cc5)nc(-c5ccc(-c6ccccc6)cc5)n4)c3)c2-c2ccccc21] 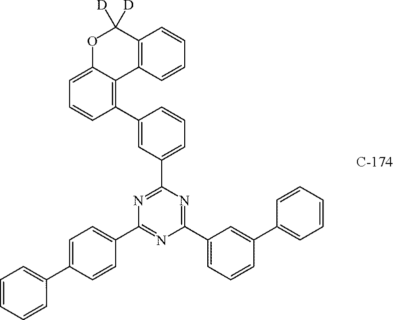 [SMILES: [2H]C1([2H])Oc2cccc(-c3cccc(-c4nc(-c5ccc(-c6ccccc6)cc5)nc(-c5cccc(-c6ccccc6)c5)n4)c3)c2-c2ccccc21] 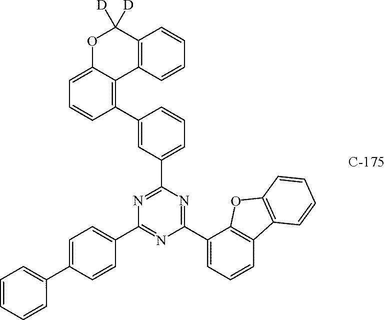 [SMILES: [2H]C1([2H])Oc2cccc(-c3cccc(-c4nc(-c5ccc(-c6ccccc6)cc5)nc(-c5cccc6c5oc5ccccc56)n4)c3)c2-c2ccccc21] 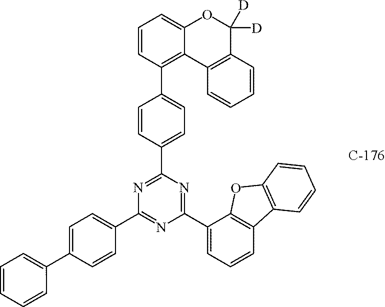 [SMILES: [2H]C1([2H])Oc2cccc(-c3ccc(-c4nc(-c5ccc(-c6ccccc6)cc5)nc(-c5cccc6c5oc5ccccc56)n4)cc3)c2-c2ccccc21] 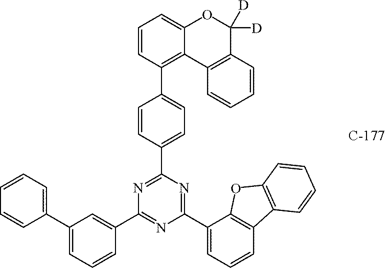 [SMILES: [2H]C1([2H])Oc2cccc(-c3ccc(-c4nc(-c5cccc(-c6ccccc6)c5)nc(-c5cccc6c5oc5ccccc56)n4)cc3)c2-c2ccccc21] 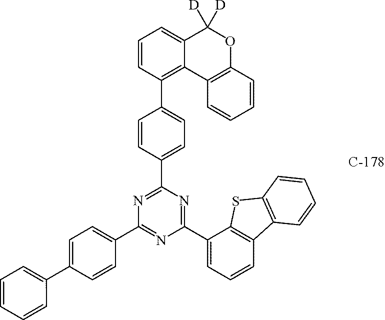 [SMILES: [2H]C1([2H])Oc2ccccc2-c2c(-c3ccc(-c4nc(-c5ccc(-c6ccccc6)cc5)nc(-c5cccc6c5sc5ccccc56)n4)cc3)cccc21] 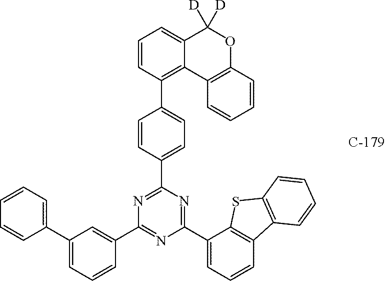 [SMILES: [2H]C1([2H])Oc2ccccc2-c2c(-c3ccc(-c4nc(-c5cccc(-c6ccccc6)c5)nc(-c5cccc6c5sc5ccccc56)n4)cc3)cccc21] 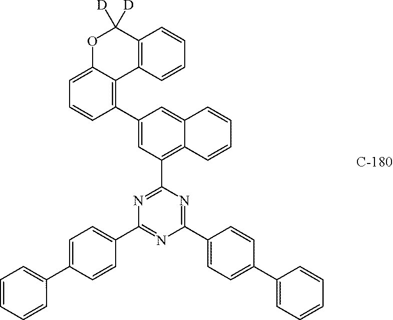 [SMILES: [2H]C1([2H])Oc2cccc(-c3cc(-c4nc(-c5ccc(-c6ccccc6)cc5)nc(-c5ccc(-c6ccccc6)cc5)n4)c4ccccc4c3)c2-c2ccccc21] 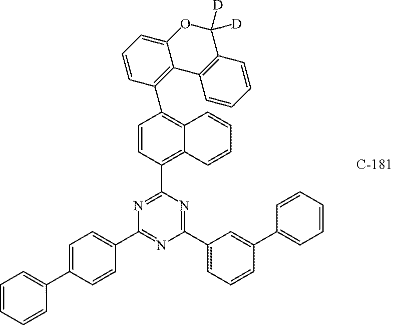 [SMILES: [2H]C1([2H])Oc2cccc(-c3ccc(-c4nc(-c5ccc(-c6ccccc6)cc5)nc(-c5cccc(-c6ccccc6)c5)n4)c4ccccc34)c2-c2ccccc21] 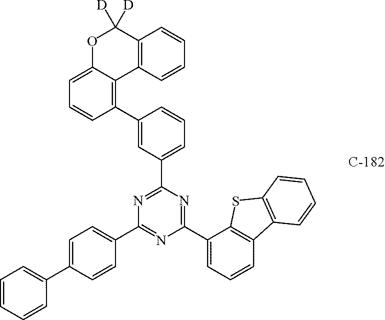 [SMILES: [2H]C1([2H])Oc2cccc(-c3cccc(-c4nc(-c5ccc(-c6ccccc6)cc5)nc(-c5cccc6c5sc5ccccc56)n4)c3)c2-c2ccccc21] 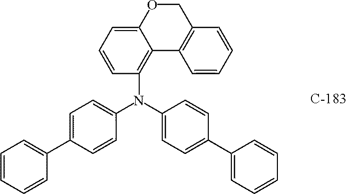 [SMILES: c1ccc(-c2ccc(N(c3ccc(-c4ccccc4)cc3)c3cccc4c3-c3ccccc3CO4)cc2)cc1] 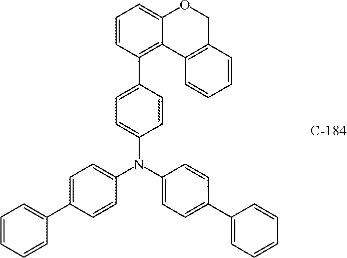 [SMILES: c1ccc(-c2ccc(N(c3ccc(-c4ccccc4)cc3)c3ccc(-c4cccc5c4-c4ccccc4CO5)cc3)cc2)cc1] 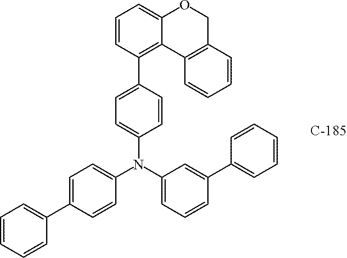 [SMILES: c1ccc(-c2ccc(N(c3ccc(-c4cccc5c4-c4ccccc4CO5)cc3)c3cccc(-c4ccccc4)c3)cc2)cc1] 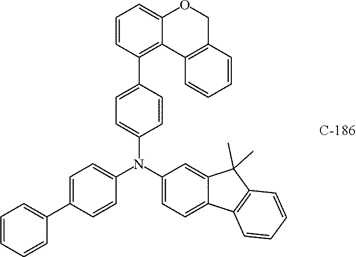 [SMILES: CC1(C)c2ccccc2-c2ccc(N(c3ccc(-c4ccccc4)cc3)c3ccc(-c4cccc5c4-c4ccccc4CO5)cc3)cc21] 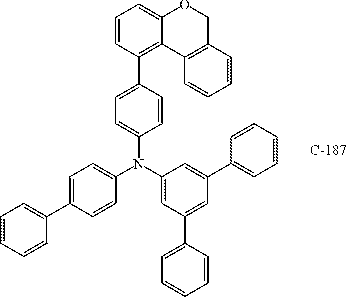 [SMILES: c1ccc(-c2ccc(N(c3ccc(-c4cccc5c4-c4ccccc4CO5)cc3)c3cc(-c4ccccc4)cc(-c4ccccc4)c3)cc2)cc1] 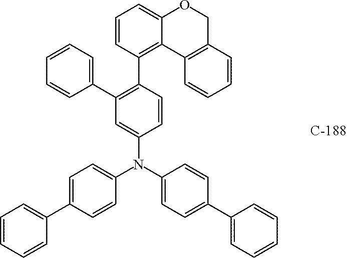 [SMILES: c1ccc(-c2ccc(N(c3ccc(-c4ccccc4)cc3)c3ccc(-c4cccc5c4-c4ccccc4CO5)c(-c4ccccc4)c3)cc2)cc1] 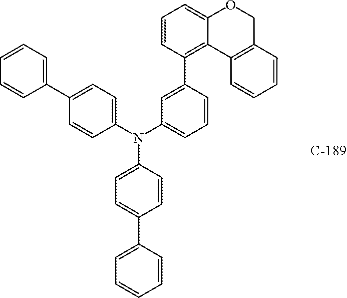 [SMILES: c1ccc(-c2ccc(N(c3ccc(-c4ccccc4)cc3)c3cccc(-c4cccc5c4-c4ccccc4CO5)c3)cc2)cc1] 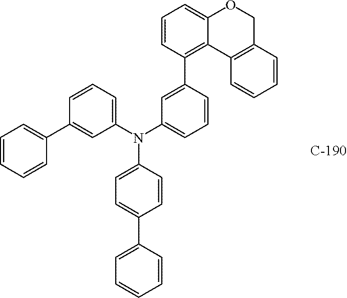 [SMILES: c1ccc(-c2ccc(N(c3cccc(-c4ccccc4)c3)c3cccc(-c4cccc5c4-c4ccccc4CO5)c3)cc2)cc1] 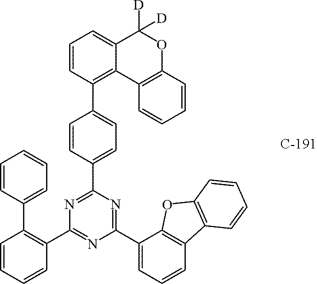 [SMILES: [2H]C1([2H])Oc2ccccc2-c2c(-c3ccc(-c4nc(-c5ccccc5-c5ccccc5)nc(-c5cccc6c5oc5ccccc56)n4)cc3)cccc21] 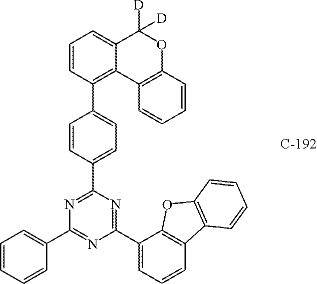 [SMILES: [2H]C1([2H])Oc2ccccc2-c2c(-c3ccc(-c4nc(-c5ccccc5)nc(-c5cccc6c5oc5ccccc56)n4)cc3)cccc21] 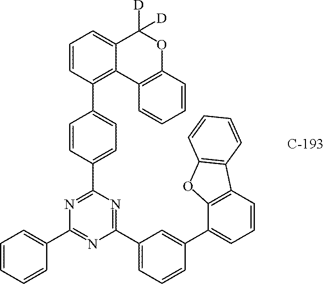 [SMILES: [2H]C1([2H])Oc2ccccc2-c2c(-c3ccc(-c4nc(-c5ccccc5)nc(-c5cccc(-c6cccc7c6oc6ccccc67)c5)n4)cc3)cccc21] 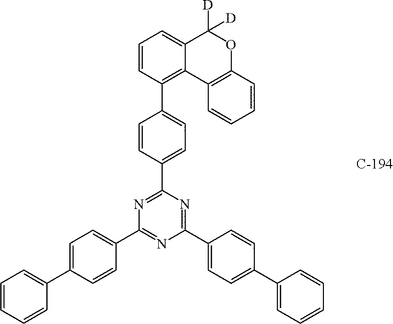 [SMILES: [2H]C1([2H])Oc2ccccc2-c2c(-c3ccc(-c4nc(-c5ccc(-c6ccccc6)cc5)nc(-c5ccc(-c6ccccc6)cc5)n4)cc3)cccc21] 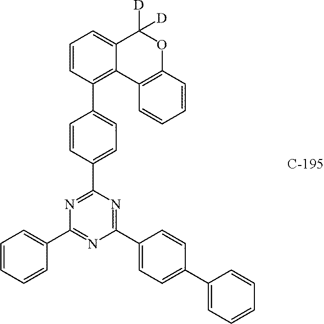 [SMILES: [2H]C1([2H])Oc2ccccc2-c2c(-c3ccc(-c4nc(-c5ccccc5)nc(-c5ccc(-c6ccccc6)cc5)n4)cc3)cccc21] 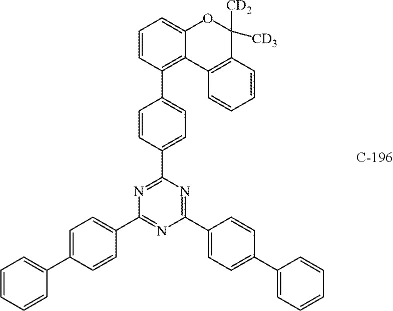 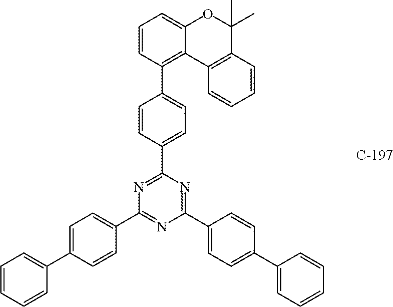 [SMILES: CC1(C)Oc2cccc(-c3ccc(-c4nc(-c5ccc(-c6ccccc6)cc5)nc(-c5ccc(-c6ccccc6)cc5)n4)cc3)c2-c2ccccc21] 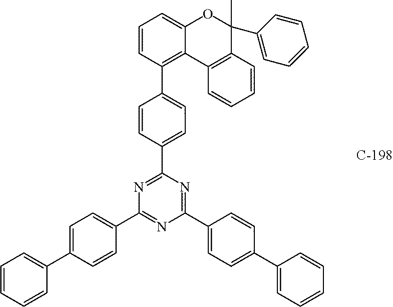 [SMILES: CC1(c2ccccc2)Oc2cccc(-c3ccc(-c4nc(-c5ccc(-c6ccccc6)cc5)nc(-c5ccc(-c6ccccc6)cc5)n4)cc3)c2-c2ccccc21] 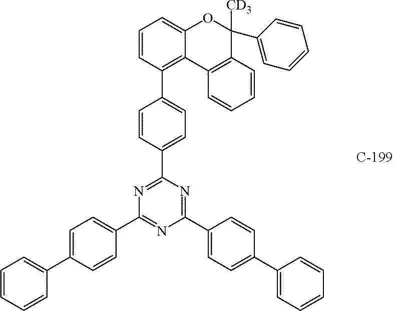 [SMILES: [2H]C([2H])([2H])C1(c2ccccc2)Oc2cccc(-c3ccc(-c4nc(-c5ccc(-c6ccccc6)cc5)nc(-c5ccc(-c6ccccc6)cc5)n4)cc3)c2-c2ccccc21] 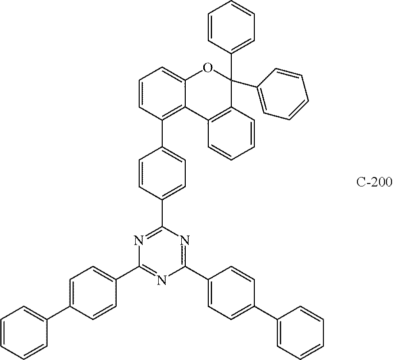 [SMILES: c1ccc(-c2ccc(-c3nc(-c4ccc(-c5ccccc5)cc4)nc(-c4ccc(-c5cccc6c5-c5ccccc5C(c5ccccc5)(c5ccccc5)O6)cc4)n3)cc2)cc1] 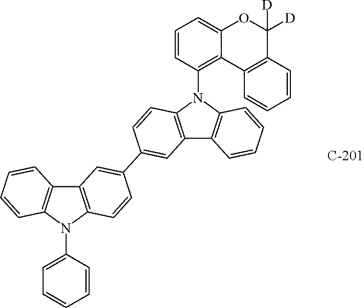 [SMILES: [2H]C1([2H])Oc2cccc(-n3c4ccccc4c4cc(-c5ccc6c(c5)c5ccccc5n6-c5ccccc5)ccc43)c2-c2ccccc21] 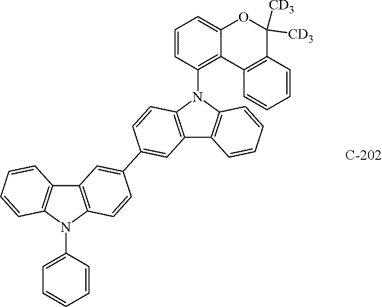 [SMILES: [2H]C([2H])([2H])C1(C([2H])([2H])[2H])Oc2cccc(-n3c4ccccc4c4cc(-c5ccc6c(c5)c5ccccc5n6-c5ccccc5)ccc43)c2-c2ccccc21] 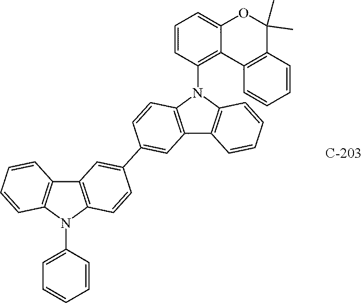 [SMILES: CC1(C)Oc2cccc(-n3c4ccccc4c4cc(-c5ccc6c(c5)c5ccccc5n6-c5ccccc5)ccc43)c2-c2ccccc21] 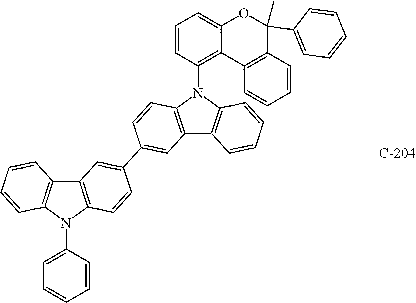 [SMILES: CC1(c2ccccc2)Oc2cccc(-n3c4ccccc4c4cc(-c5ccc6c(c5)c5ccccc5n6-c5ccccc5)ccc43)c2-c2ccccc21] 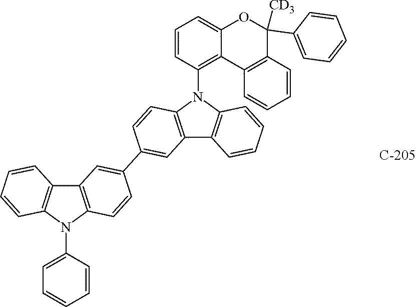 [SMILES: [2H]C([2H])([2H])C1(c2ccccc2)Oc2cccc(-n3c4ccccc4c4cc(-c5ccc6c(c5)c5ccccc5n6-c5ccccc5)ccc43)c2-c2ccccc21] 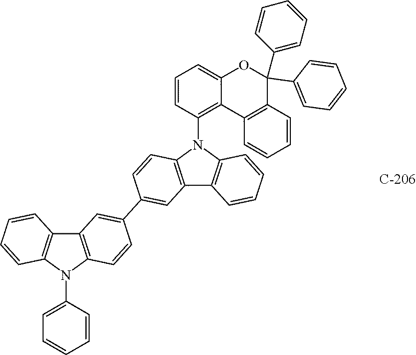 [SMILES: c1ccc(-n2c3ccccc3c3cc(-c4ccc5c(c4)c4ccccc4n5-c4cccc5c4-c4ccccc4C(c4ccccc4)(c4ccccc4)O5)ccc32)cc1] 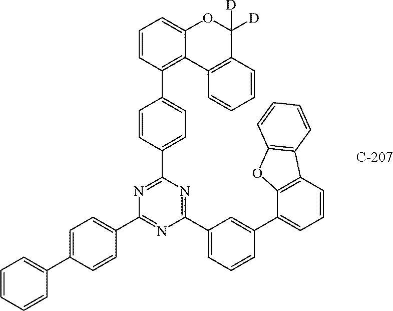 [SMILES: [2H]C1([2H])Oc2cccc(-c3ccc(-c4nc(-c5ccc(-c6ccccc6)cc5)nc(-c5cccc(-c6cccc7c6oc6ccccc67)c5)n4)cc3)c2-c2ccccc21] 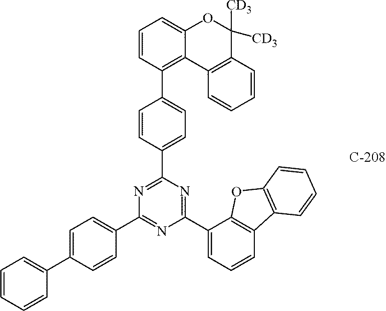 [SMILES: [2H]C([2H])([2H])C1(C([2H])([2H])[2H])Oc2cccc(-c3ccc(-c4nc(-c5ccc(-c6ccccc6)cc5)nc(-c5cccc6c5oc5ccccc56)n4)cc3)c2-c2ccccc21] 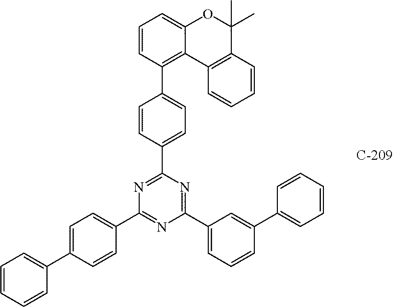 [SMILES: CC1(C)Oc2cccc(-c3ccc(-c4nc(-c5ccc(-c6ccccc6)cc5)nc(-c5cccc(-c6ccccc6)c5)n4)cc3)c2-c2ccccc21] 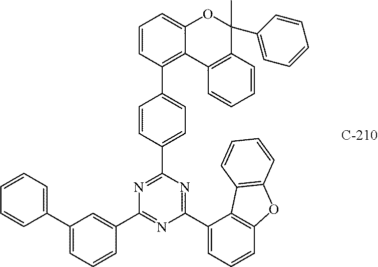 [SMILES: CC1(c2ccccc2)Oc2cccc(-c3ccc(-c4nc(-c5cccc(-c6ccccc6)c5)nc(-c5cccc6oc7ccccc7c56)n4)cc3)c2-c2ccccc21] 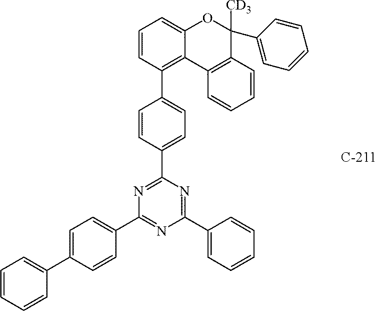 [SMILES: [2H]C([2H])([2H])C1(c2ccccc2)Oc2cccc(-c3ccc(-c4nc(-c5ccccc5)nc(-c5ccc(-c6ccccc6)cc5)n4)cc3)c2-c2ccccc21] 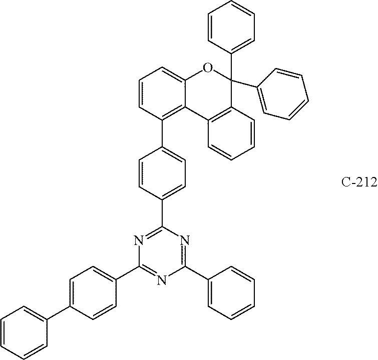 [SMILES: c1ccc(-c2ccc(-c3nc(-c4ccccc4)nc(-c4ccc(-c5cccc6c5-c5ccccc5C(c5ccccc5)(c5ccccc5)O6)cc4)n3)cc2)cc1] 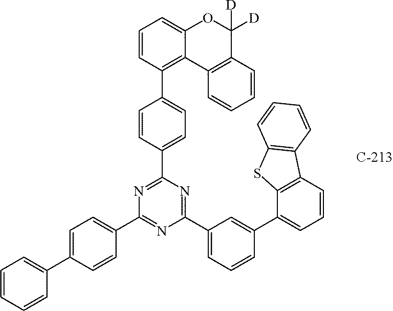 [SMILES: [2H]C1([2H])Oc2cccc(-c3ccc(-c4nc(-c5ccc(-c6ccccc6)cc5)nc(-c5cccc(-c6cccc7c6sc6ccccc67)c5)n4)cc3)c2-c2ccccc21] 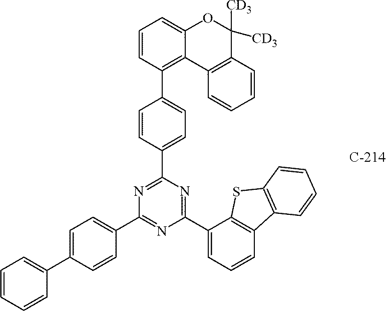 [SMILES: [2H]C([2H])([2H])C1(C([2H])([2H])[2H])Oc2cccc(-c3ccc(-c4nc(-c5ccc(-c6ccccc6)cc5)nc(-c5cccc6c5sc5ccccc56)n4)cc3)c2-c2ccccc21] 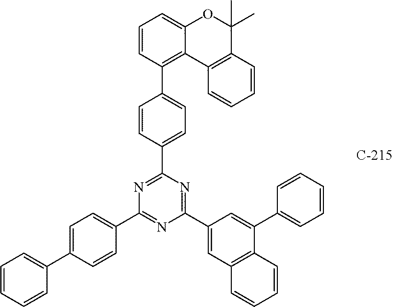 [SMILES: CC1(C)Oc2cccc(-c3ccc(-c4nc(-c5ccc(-c6ccccc6)cc5)nc(-c5cc(-c6ccccc6)c6ccccc6c5)n4)cc3)c2-c2ccccc21] 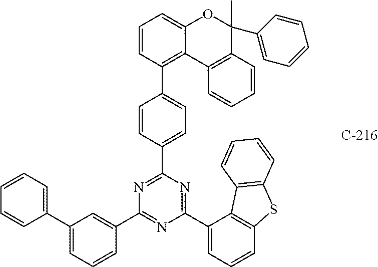 [SMILES: CC1(c2ccccc2)Oc2cccc(-c3ccc(-c4nc(-c5cccc(-c6ccccc6)c5)nc(-c5cccc6sc7ccccc7c56)n4)cc3)c2-c2ccccc21] 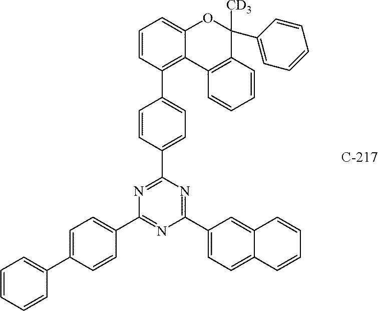 [SMILES: [2H]C([2H])([2H])C1(c2ccccc2)Oc2cccc(-c3ccc(-c4nc(-c5ccc(-c6ccccc6)cc5)nc(-c5ccc6ccccc6c5)n4)cc3)c2-c2ccccc21] 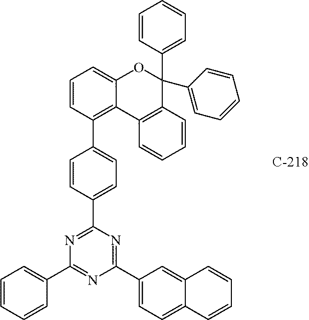 [SMILES: c1ccc(-c2nc(-c3ccc(-c4cccc5c4-c4ccccc4C(c4ccccc4)(c4ccccc4)O5)cc3)nc(-c3ccc4ccccc4c3)n2)cc1] 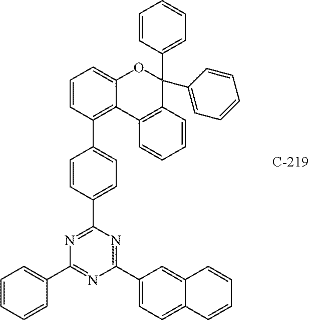 [SMILES: c1ccc(-c2nc(-c3ccc(-c4cccc5c4-c4ccccc4C(c4ccccc4)(c4ccccc4)O5)cc3)nc(-c3ccc4ccccc4c3)n2)cc1] 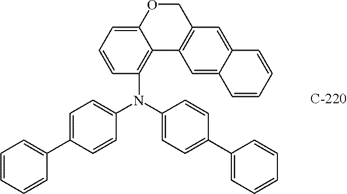 [SMILES: c1ccc(-c2ccc(N(c3ccc(-c4ccccc4)cc3)c3cccc4c3-c3cc5ccccc5cc3CO4)cc2)cc1] 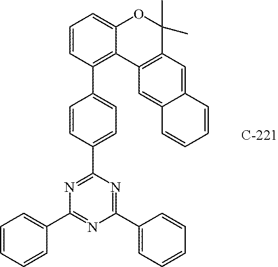 [SMILES: CC1(C)Oc2cccc(-c3ccc(-c4nc(-c5ccccc5)nc(-c5ccccc5)n4)cc3)c2-c2cc3ccccc3cc21] 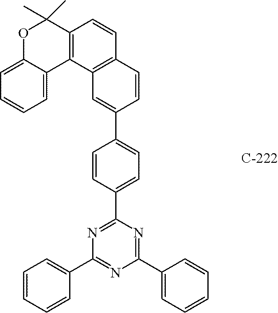 [SMILES: CC1(C)Oc2ccccc2-c2c1ccc1ccc(-c3ccc(-c4nc(-c5ccccc5)nc(-c5ccccc5)n4)cc3)cc21] 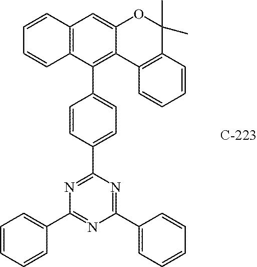 [SMILES: CC1(C)Oc2cc3ccccc3c(-c3ccc(-c4nc(-c5ccccc5)nc(-c5ccccc5)n4)cc3)c2-c2ccccc21] 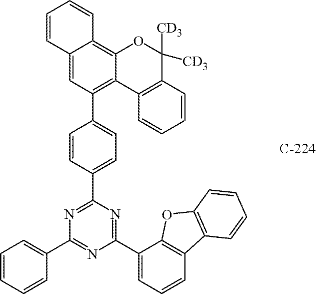 [SMILES: [2H]C([2H])([2H])C1(C([2H])([2H])[2H])Oc2c(c(-c3ccc(-c4nc(-c5ccccc5)nc(-c5cccc6c5oc5ccccc56)n4)cc3)cc3ccccc23)-c2ccccc21] 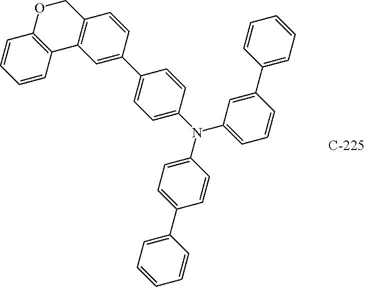 [SMILES: c1ccc(-c2ccc(N(c3ccc(-c4ccc5c(c4)-c4ccccc4OC5)cc3)c3cccc(-c4ccccc4)c3)cc2)cc1] 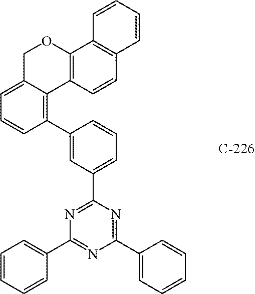 [SMILES: c1ccc(-c2nc(-c3ccccc3)nc(-c3cccc(-c4cccc5c4-c4ccc6ccccc6c4OC5)c3)n2)cc1] 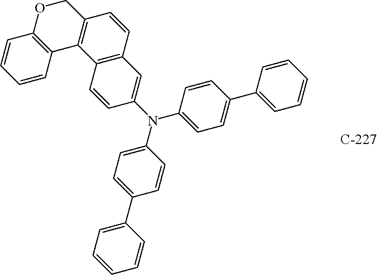 [SMILES: c1ccc(-c2ccc(N(c3ccc(-c4ccccc4)cc3)c3ccc4c5c(ccc4c3)COc3ccccc3-5)cc2)cc1] 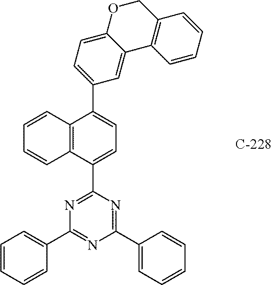 [SMILES: c1ccc(-c2nc(-c3ccccc3)nc(-c3ccc(-c4ccc5c(c4)-c4ccccc4CO5)c4ccccc34)n2)cc1] 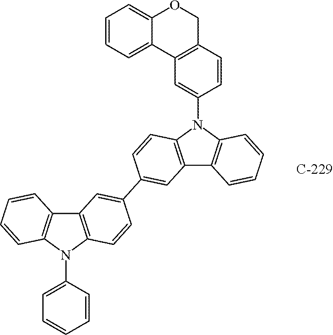 [SMILES: c1ccc(-n2c3ccccc3c3cc(-c4ccc5c(c4)c4ccccc4n5-c4ccc5c(c4)-c4ccccc4OC5)ccc32)cc1] 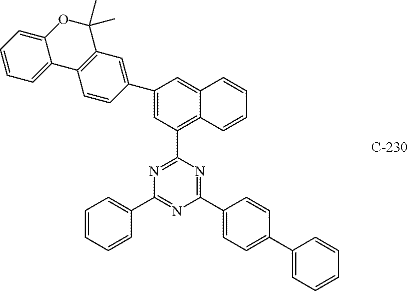 [SMILES: CC1(C)Oc2ccccc2-c2ccc(-c3cc(-c4nc(-c5ccccc5)nc(-c5ccc(-c6ccccc6)cc5)n4)c4ccccc4c3)cc21] 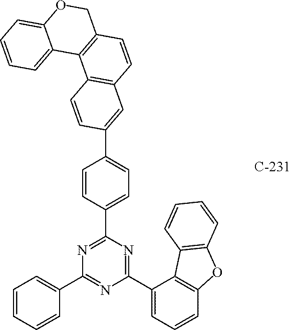 [SMILES: c1ccc(-c2nc(-c3ccc(-c4ccc5c6c(ccc5c4)COc4ccccc4-6)cc3)nc(-c3cccc4oc5ccccc5c34)n2)cc1] 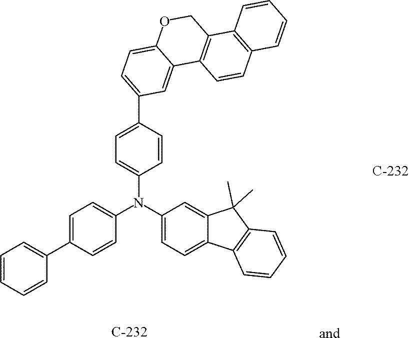 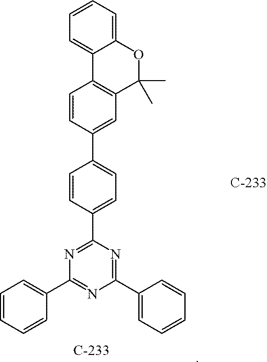
.

10. An organic electroluminescent device comprising an anode, a cathode, and at least one light-emitting layer between the anode and the cathode, wherein the at least one light-emitting layer comprises the plurality of host materials according to claim 1.

11. An organic electroluminescent material comprising the organic electroluminescent compound according to claim 7.

12. An organic electroluminescent device comprising the organic electroluminescent compound according to claim 7.
